(19) United States
(12) Patent Application Publication
(10) Pub. No.: US 2024/0415014 A1
(43) Pub. Date: Dec. 12, 2024

(54) ORGANIC MOLECULE LIGHT EMITTERS

(71) Applicant: The Governing Council of the University of Toronto, Toronto (CA)

(72) Inventors: Alán Aspuru-Guzik, Toronto (CA); AkshatKumar Nigam, Stanford, CA (US); Robert Pollice, Toronto (CA); Pascal Thomas Friederich, Neckarsulm (DE); Benjamin Bangjie Ding, Toronto (CA)

(73) Assignee: The Governing Council of the University of Toronto, Toronto (CA)

(21) Appl. No.: 18/671,224

(22) Filed: May 22, 2024

Related U.S. Application Data
(60) Provisional application No. 63/469,178, filed on May 26, 2023.

(51) Int. Cl. H10K 85/60; C07D 487/04; C07D 519/00; C09K 11/06; H01S 5/36

(52) U.S. Cl. CPC H10K 85/6572; C07D 487/04; C07D 519/00; C09K 11/06; H01S 5/36; H10K 85/654; H10K 85/657; C09K 2211/1018

(57) ABSTRACT

The present application relates to compounds (e.g. Formulae III, V, VII, IX, and XII) having a negative singlet-triplet gap and a positive oscillator strength. The present application also relates to use of the compounds of the present application in photocatalysis and in OLEDs as emitters and/or dopants.

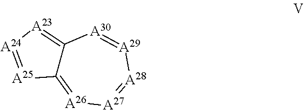

### CROSS-REFERENCE TO RELATED APPLICATIONS

The present application claims the benefit of priority from co-pending U.S. provisional patent application No. 63/469,178 filed on May 26, 2023, the contents of which are incorporated herein by reference in their entirety.

### FIELD

The present application relates to organic compounds with a negative singlet-triplet gap and a positive oscillator strength. The present application further relates to the use of the compounds as emitters and/or dopants in organic light-emitting diodes (OLED) and in photocatalysis.

### INTRODUCTION

The vast majority of closed-shell molecules have a first excited triplet state that is lower in energy than the first excited singlet state, which can be predicted by applying Hund's first rule to electronic excited states, 1 and can be rationalized based on the Pauli Exclusion principle² due to a diminished exchange energy in the triplet compared to the singlet state.³ Only a few exceptions are known, most of which are organic⁴⁻¹⁰ but at least one is inorganic¹¹ and some are organometallic.¹² They are sometimes referred to as inverted singlet-triplet gap (INVEST or STG) molecules,^{13,14} and they all stem from stabilization of the first excited singlet via electron correlation relative to the first excited triplet. Simplified models rationalize these violations of Hund's first rule either via the presence of appreciable dynamic spin polarization,¹⁵⁻¹⁷ or via significant contributions of double excitations.^{13,18} Recently, azaphenalenes have gained considerable attention in the field of organic electronic materials because it was realized that they likely have an inverted STG, i.e., they violate Hund's first rule in their first excited states, and their high stabilities and tunabilities make them ideal candidates for applications such as photocatalysts and organic light-emitting diodes.^{13,14,19-22} Nevertheless, apart from azaphenalenes, most other suspected exceptions to Hund's first rule are highly unstable and reactive, severely limiting their potential in real-life applications.

Accordingly, there is a need to develop organic INVEST molecules as well as a method to design and develop such molecules.

### SUMMARY

A computational study in which the space of organic compounds violating Hund's first rule in the first electronic excited states, i.e., organic INVEST molecules, was systematically explored herein. By formulating simple construction rules, termed the Ring-Bonds-Substitutions (RiBS) ruleset, a dataset was built with 69,201 potential INVEST candidates and their excited state properties predicted with computational methods accounting for electron correlation in the excited states. It was found that a significant fraction of the candidates were indeed predicted to have an inverted STG between their first excited states, thereby greatly expanding the space of potential INVEST molecules. Additionally, the stability and synthesizability of all the compound families investigated was assessed to find suitable candidates for future applications as organic electronic materials and, more specifically, organic emitters.

Accordingly, in one aspect, the present application includes a compound of Formula III or a salt and/or tautomer thereof

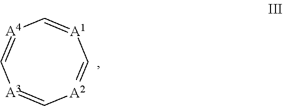

wherein:
A¹, A², A³ and A⁴ are each independently selected from C and N, and 0, 2, or 4 of A¹, A², A³ and A⁴ are N; and
1 to 6 of the carbons of the compound of Formula III are each independently substituted with CN or NH₂.

In another aspect, the present application includes a compound of Formula VII or a salt and/or tautomer thereof.

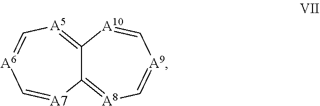

wherein:
A⁵, A⁶, A⁷, A⁸, A⁹ and A¹º are each independently selected from C and N, and 1 to 5 of A⁵, A⁶, A⁷, A⁸, A⁹ and A¹⁰ are N; and 1 to 6 of the carbons of the compound of Formula VII are each independently substituted with CN or NH₂.

In another aspect, the present application includes a compound of Formula IX or a salt and/or tautomer thereof

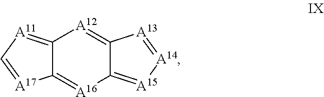

wherein:
A¹¹, A¹², A¹³, A¹⁴, A¹⁵, A¹⁶ and A¹⁷ are each independently selected from C and N, and 0 to 6 of A¹¹, A¹², A¹³, A¹⁴, A¹⁵, A¹⁶ and A¹⁷ are N; and 0 to 6 of the carbons of the compound of Formula IX are each independently substituted with CN or NH₂.

In another aspect, the present application includes a compound of Formula XII or a salt and/or tautomer thereof

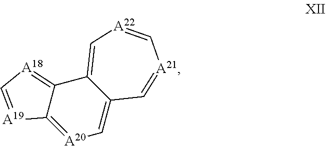

wherein:
A¹⁸, A¹⁹, A²⁰, A²¹ and A²² are each independently selected from C and N, and 0 to 5 of A¹⁸, A¹⁹, A²⁰, A²¹ and A²² are N; and
0 to 6 carbons of the compound of Formula XII are each independently substituted with CN or NH₂.

In another aspect, the present application includes a compound of Formula V or a salt and/or tautomer thereof

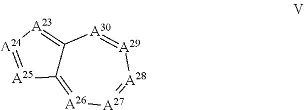

wherein:
A²³, A²⁴, A²⁵, A²⁶, A²⁷, A²⁸, A²⁹ and A³⁰ are each independently selected from C and N, and 0 to 4 of A²³, A²⁴, A²⁵, A²⁶, A²⁷, A²⁸, A²⁹ and A³⁰ are N; and each of A²³, A²⁴, A²⁵, A²⁶, A²⁷, A²⁸, A²⁹ and A³⁰ that is a C is independently unsubstituted or substituted with a substituent of Formula Va

-L-R1 Va
wherein:
L is selected from a bond, O, S, NH, —CR^{a}═, —N═, C(O), C₂₋₆alkenylene, C₂₋₆alkenyleneO and C₂₋₆alkenyleneS;
when L is a bond, R¹ is selected from H, CN and halo;
when L is other than a bond, R¹ is selected from H, C₅₋₂₀heteroaryl, C₅₋₂₀cycloalkenyl, C₆₋₂₀aryl, C₄₋₂₀heterocyclyl and C₂-C₆alkenyl, the latter 5 groups being unsubstituted or substituted with one or more groups independently selected from halo, CN, OH, NH₂, SH, C(O)H, C₂₋₄alkenyl, C₂₋₄fluoroalkenyl, NHC₂₋₄alkenyl, NHC₂₋₄fluoroalkenyl, OC₂₋₄alkenyl, SC₂₋₄fluoroalkenyl, NH₂-substituted C₂₋₄alkenyl, C(O)H, OC(O)H, NHC(O)H, SC(O)H, and C(O)NH₂, and/or
with one or two groups independently selected from =CHR^{a}, C₅-C₀heteroaryl, C₅₋₂₀cycloalkenyl, C₆₋₂₀aryl, C₄₋₂₀heterocyclyl, OC₅₋₂₀heteroaryl, OC₅₋₂₀cycloalkenyl, OC₆₋₂₀aryl, OC₄₋₂₀heterocyclyl, NHC₅₋₂₀heteroaryl, NHC₅₋₂₀cycloalkenyl, NHC₆₋₂₀aryl, NHC₄₋₂₀heterocyclyl, SC₅₋₂₀heteroaryl, SC₅₋₂₀cycloalkenyl, SC₆₋₂₀aryl, SC₄₋₂₀heterocyclyl, C(O)C₅₋₂₀heteroaryl, C(O)C₅₋₂₀cycloalkenyl, C(O)C₆₋₂₀aryl, C(O)C₄₋₂₀heterocyclyl, C₂₋₄alkenyleneC₅₋₂₀heteroaryl, C₂₋₄alkenyleneC₅₋₂₀cycloalkenyl, C₂₋₄alkenyleneC₆₋₂₀aryl and C₂₋₄alkenyleneC₄₋₂₀heterocyclyl, in which each cyclic group is unsubstituted or substituted with one or more groups independently selected from NH₂, CN, OH, SH, halo, C₂₋₄alkenyl and C(O)H;
R^{a} is selected from H, C₅₋₁₀heteroaryl, and C₆₋₁₀aryl, the latter two groups being unsubstituted or substituted with one or more group independently selected from NH₂, CN, OH, SH, halo, C₂₋₄alkenyl and C(O)H, and
the compound has a negative singlet-triplet gap and an oscillator strength greater than or equal to about 0.01.

In another aspect, the present application includes an organic light-emitting diode comprising at least one compound of the present application.

In another aspect, the present application includes a photocatalyst comprising at least one compound of the present application.

In another aspect, the present application includes a triplet quencher comprising at least one compound of the present application.

In another aspect, the present application also includes a use of a compound disclosed in an organic light-emitting diode.

In another aspect, the compound is used as an emitter or a dopant.

In another aspect, the present application also includes a method of preparing an organic light-emitting diode comprising providing at least one compound disclosed herein as an emitter or a dopant.

In another aspect, the present application also includes an organic-light emitting diode comprising at least one compound disclosed herein.

In another aspect, the present application includes a use of a compound a disclosed herein as a photocatalyst.

In another aspect, the present application includes a method of performing photocatalysis comprising contacting at least one compound disclosed herein with a mixture requiring a photocatalyst and performing a photocatalytic transformation on the mixture.

In another aspect, the present application includes a use of a compound disclosed herein in the generation of organic laser.

In another aspect, the present application includes a method of generating organic laser comprising providing at least one compound disclosed herein as a light emitter.

In another aspect, the present application also includes an organic-laser comprising at least one compound disclosed herein.

In another aspect, the present application includes a use of a compound disclosed herein in the enhancement of photostability.

### DRAWINGS

The embodiments of the application will now be described in greater detail with reference to the attached drawings in which:

FIG. 1 shows of the core structures of the compound space investigated which covers 16 exemplary families of compounds (I-XVI).

FIG. 2 shows property ranges with box and whisker plots of the entire set of compounds at the ADC(2)/cc-pVDZ level of theory for all of the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 3 shows property histograms of the entire set of exemplary compounds at the ADC(2)/cc-pVDZ level of theory (panels A-C) and alpha shapes of the corresponding point clouds for each family (D-F).

FIG. 4 shows planarity descriptor ranges with box and whisker plots of the entire set of compounds divided into the exemplary molecular families considered. Vertical lines mark the properties of single molecules. Planarity descriptor values of 1 indicate perfect planarity and the higher the deviation from that value the lower the planarity.

FIG. 5 shows normalized fractional occupation number weighted density (FOD) integral ranges with box and whisker plots of the entire set of compounds divided into the exemplary molecular families considered. Vertical lines mark the properties of single molecules.

FIG. 6 shows ranges of the property corrections due to geometric relaxation with box and whisker plots of the entire set of compounds at the calibrated RKS-ωB2PLYP′/def2-SVP//B3LYP/6-31G* level of theory divided into the exemplary molecular families considered. Vertical lines mark the properties of single molecules (panels A-C) and property-property correction histograms of the entire set of compounds at the ADC(2)/cc-pVDZ (property) and calibrated RKS-ωB2PLYP′/def2-SVP//B3LYP/6-31G* (correction) levels of theory (panels D-F).

FIG. 7 shows an illustration of the (partially) disjoint frontier orbitals in both [4n] and [4n+2] π-systems at stage 1 and the effect of introducing a bond in stage 2 of the RiBS ruleset. Orbital coefficients are based on the Huckel approximation. In [4n+2] π-systems, only one of the two degenerate highest occupied molecular orbitals (HOMOs) and one of the two degenerate lowest unoccupied molecular orbitals (LUMOs) are displayed, namely the ones that become the frontier orbitals after introducing bonds.

FIG. 8 shows property ranges with box and whisker plots of the entire set of compounds at the SOS-ADC(2)/cc-pVDZ level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 9 shows property histograms of the entire set of exemplary compounds at the SOS-ADC(2)/cc-pVDZ level of theory (panels A-C) and alpha shapes of the corresponding point clouds for each family (panels D-F).

FIG. 10 shows property ranges with box and whisker plots of the entire set of compounds at the RKS-ωB2PLYP′/def2-SVP level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 11 shows property histograms of the entire set of exemplary compounds at the RKS-ωB2PLYP′/def2-SVP level of theory (panels A-C) and alpha shapes of the corresponding point clouds for each family panels (D-F).

FIG. 12 shows property ranges with box and whisker plots of the entire set of compounds at the UKS-ωB2PLYP/def2-SVP level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 13 shows property histograms of the entire set of exemplary compounds at the UKS-ωB2PLYP/def2-SVP level of theory (panels A-C) and alpha shapes of the corresponding point clouds for each family (panels D-F).

FIG. 14 shows property ranges with box and whisker plots of the entire set of compounds at the SF-PBE50/def2-SVP level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 15 shows property histograms of the entire set of exemplary compounds at the SF-PBE50/def2-SVP level of theory (panels A-C) and alpha shapes of the corresponding point clouds for each family (panels D-F).

FIG. 16 shows property ranges with box and whisker plots of the entire set of compounds at the CC2/cc-pVDZ level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 17 shows property histograms of the entire set of exemplary compounds at the CC2/cc-pVDZ level of theory (panel A) and alpha shapes of the corresponding point clouds for each family (panel B).

FIG. 18 shows property ranges with box and whisker plots of the entire set of compounds at the SOS—CC2/cc-pVDZ level of theory divided into the exemplary molecular families considered. Vertical lines mark the properties of single molecules. Panel A plots the singlet-triplet gap ΔE(S₁−T₁) (eV) and panel B plots the vertical excitation energy ΔS(S₀−S₁) (eV) for the molecular families.

FIG. 19 shows property histograms of the entire set of exemplary compounds at the SOS—CC2/cc-pVDZ level of theory (panel A) and alpha shapes of the corresponding point clouds for each family (panel B).

FIG. 20 shows property ranges with box and whisker plots of the entire set of compounds at the CIS(D)/cc-pVDZ level of theory divided into the exemplary molecular families considered. Vertical lines mark the properties of single molecules. A plots the singlet-triplet gap ΔE(S₁−T₁) (eV), B plots the oscillator strength f₁₂, and C plots the vertical excitation energy ΔS(S₀−S₁) (eV) for the molecular families.

FIG. 21 shows property histograms of the entire set of exemplary compounds at the CIS(D)/cc-pVDZ level of theory (panels A-C) and alpha shapes of the corresponding point clouds for each family (panels D-F).

FIG. 22 shows property ranges with box and whisker plots of the entire set of compounds at the SOS—CIS(D)/cc-pVDZ level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 23 shows property histograms of the entire set of exemplary compounds at the SOS—CIS(D)/cc-pVDZ level of theory (panels A-C) and alpha shapes of the corresponding point clouds for each family (panels D-F).

FIG. 24 shows property ranges with box and whisker plots of the entire set of compounds at the SCS—CIS(D)/cc-pVDZ level of theory divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 25 shows property histograms of the entire set of exemplary compounds at the SCS—CIS(D)/cc-pVDZ level of theory (panels A-C) and alpha shapes of the corresponding point clouds for each family (panels D-F).

FIG. 26 shows correlation of excited state properties of the entire set of exemplary compounds at the SOS-ADC(2)/cc-pVDZ level of theory against the corresponding properties at the ADC(2)/cc-pVDZ level of theory. The black line indicates what perfect correspondence between the two properties would look like.

FIG. 27 shows correlation of excited state properties of the entire set of exemplary compounds at the RKS-ωB2PLYP′/def2-SVP level of theory against the corresponding properties at the ADC(2)/cc-pVDZ level of theory. The black line indicates what perfect correspondence between the two properties would look like.

FIG. 28 shows correlation of excited state properties of the entire set of exemplary compounds at the UKS-ωB2PLYP/def2-SVP level of theory against the corresponding properties at the ADC(2)/cc-pVDZ level of theory. The black line indicates what perfect correspondence between the two properties would look like.

FIG. 29 shows correlation of excited state properties of the entire set of exemplary compounds at the SF-PBE50/def2-SVP level of theory against the corresponding properties at the ADC(2)/cc-pVDZ level of theory. The black line indicates what perfect correspondence between the two properties would look like.

FIG. 30 shows correlation of excited state properties of the entire set of exemplary compounds at the CC2/cc-pVDZ level of theory against the corresponding properties at the ADC(2)/cc-pVDZ level of theory. The black line indicates what perfect correspondence between the two properties would look like.

FIG. 31 shows Correlation of excited state properties of the entire set of exemplary compounds at the SOS—CC2/cc-pVDZ level of theory against the corresponding properties at the ADC(2)/cc-pVDZ level of theory. The black line indicates what perfect correspondence between the two properties would look like.

FIG. 32 shows correlation of excited state properties of the entire set of exemplary compounds at the CIS(D)/cc-pVDZ level of theory against the corresponding properties at the ADC(2)/cc-pVDZ level of theory. The black line indicates what perfect correspondence between the two properties would look like.

FIG. 33 shows correlation of excited state properties of the entire set of exemplary compounds at the SOS—CIS(D)/cc-pVDZ level of theory against the corresponding properties at the ADC(2)/cc-pVDZ level of theory. The black line indicates what perfect correspondence between the two properties would look like.

FIG. 34 shows correlation of excited state properties of the entire set of exemplary compounds at the SCS—CIS(D)/cc-pVDZ level of theory against the corresponding properties at the ADC(2)/cc-pVDZ level of theory. The black line indicates what perfect correspondence between the two properties would look like.

FIG. 35 shows property histograms of the entire set of exemplary compounds at the ADC(2)/cc-pVDZ level of theory (panels A-C) and alpha shapes of the corresponding point clouds for each family (panels D-F).

FIG. 36 shows property histograms of the entire set of exemplary compounds at the ADC(2)/cc-pVDZ level of theory together with the integral of the fractional occupation number weighted electron density over all space normalized by the corresponding total number of electrons (N_{FOD}/Nₑₗ) (panels A-C) and alpha shapes of the corresponding point clouds for each family (panels D-F).

FIG. 37 shows calibration of excited state properties at the ωB2PLYP′ level of theory against the corresponding properties at the ADC(2) level of theory. The data points are depicted as 2-dimensional property distribution densities showing the number of exemplary molecules in each of the small property windows. The black lines depict the robust linear regression results using a Theil-Sen estimator.

FIG. 38 shows property ranges with box and whisker plots of the entire set of compounds at the ADC(2)/cc-pVDZ level of theory corrected for excited state geometry relaxation divided into the exemplary molecular families considered. Vertical lines mark the properties of single molecules. Panel A plots the singlet-triplet gap ΔE(S₁−T₁) (eV), panel B plots the oscillator strength f₁₂, and panel C plots the vertical excitation energy ΔS(S₀−S₁) (eV) for the molecular families.

FIG. 39 shows property histograms for the entire set of exemplary compounds comparing the vertical and adiabatic excited state properties at the ADC(2)/cc-pVDZ level of theory (panels A-C) and alpha shapes of the corresponding point clouds for each family (panels D-F).

FIG. 40 shows property histograms of the entire set of exemplary compounds at the ADC(2)/cc-pVDZ level of theory corrected for excited state geometry relaxation (panels A-C) and alpha shapes of the corresponding point clouds for each family (panels D-F).

FIG. 41 shows a flowchart of a method of accelerated molecular discovery according to an example.

FIG. 42 shows a flowchart of a method of artificial molecular design according to an example.

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DESCRIPTION OF VARIOUS EMBODIMENTS

### I. Definitions

Unless otherwise indicated, the definitions and embodiments described in this and other sections are intended to be applicable to all embodiments and aspects of the present application herein described for which they are suitable as would be understood by a person skilled in the art.

The term “and/or” as used herein means that the listed items are present, or used, individually or in combination. In effect, this term means that “at least one of” or “one or more” of the listed items is used or present.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

As used in this application and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “consisting” and its derivatives as used herein are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The term “suitable” as used herein means that the selection of the particular compound or conditions would depend on the specific synthetic manipulation to be performed, the identity of the molecule(s) to be transformed and/or the specific use for the compound, but the selection would be well within the skill of a person trained in the art.

In embodiments of the present application, the compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of compounds of the present application having an alternate stereochemistry. It is intended that any optical isomers, as separated, pure or partially purified optical isomers or racemic mixtures thereof are included within the scope of the present application.

The compounds described herein may also exist in different tautomeric forms and it is intended that any tautomeric forms which the compounds form, as well as mixtures thereof, are included within the scope of the present application.

The compounds described herein may further exist in varying polymorphic forms and it is contemplated that any polymorphs, or mixtures thereof, which form are included within the scope of the present application.

The present description refers to a number of chemical terms and abbreviations used by those skilled in the art. Nevertheless, definitions of selected terms are provided for clarity and consistency.

The terms “about”, “substantially” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The suffix “ene”, when appended to a chemical group means that the group contains substituents on two of its ends, that is, it is bivalent.

The term “alkenyl” as used herein, whether it is used alone or as part of another group, means straight or branched chain, unsaturated alkyl groups containing at least one double bond. The number of carbon atoms that are possible in the referenced alkylene group are indicated by the prefix “Cₙ₁₋ₙ₂”. For example, the term C₂₋₆alkenyl means an alkenyl group having 2, 3, 4, 5 or 6 carbon atoms and at least one double bond.

The term “fluoroalkenyl” as used herein means an alkenyl group in which one or more, including all, of the hydrogen atoms are replaced with a fluorine atom.

The term “NH₂-substutited” as used herein in reference to a specific chemical group means that the group is substituted with one or more NH₂ groups.

The term “cycloalkenyl,” as used herein, whether it is used alone or as part of another group, means an unsaturated, non-aromatic carbocyclic group containing from 4 to 20 carbon atoms, one or more rings and one or more double bonds. The number of carbon atoms that are possible in the referenced cycloalkenyl group are indicated by the numerical prefix “Cₙ₁₋ₙ₂”. For example, the term C₄₋₁₀cycloalkenyl means a cycloalkyl group having 3, 4, 5, 6, 7, 8, 9 or 10 carbon atoms.

The term “aryl” as used herein, whether it is used alone or as part of another group, refers to carbocyclic groups containing at least one aromatic ring and contains 6 to 20 carbon atoms.

The term “heterocyclyl” as used herein, whether it is used alone or as part of another group, refers to cyclic groups containing at least one non-aromatic ring containing from 5 to 20 atoms in which one or more of the atoms are a heteroatom selected from O, S, P and N and the remaining atoms are C. Heterocyclyl groups are either saturated or unsaturated (i.e. contain one or more double bonds). When a heterocyclyl group contains the prefix Cₙ₁₋ₙ₂ this prefix indicates the number of carbon atoms in the corresponding carbocyclic group, in which one or more, suitably 1 to 5, of the ring atoms is replaced with a heteroatom selected from O, S, P and N and the remaining atoms are C. Heterocyclyl groups are optionally benzofused.

The term “heteroaryl” as used herein, whether it is used alone or as part of another group, refers to cyclic groups containing at least one heteroaromatic ring containing 5-20 atoms in which one or more of the atoms are a heteroatom selected from O, S, P and N and the remaining atoms are C. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteroatom as defined above. Heteroaryl groups are optionally benzofused.

All cyclic groups, including aryl, heteroaryl, heterocyclyl, heterocycloalkyl and cycloalkyl groups, contain one or more than one ring (i.e. are polycyclic). When a cyclic group contains more than one ring, the rings may be fused, bridged or spirofused.

The term “benzofused” as used herein refers to a polycyclic group in which a benzene ring is fused with another ring.

A first ring being “fused” with a second ring means the first ring and the second ring share two adjacent atoms there between.

A first ring being “bridged” with a second ring means the first ring and the second ring share two non-adjacent atoms there between.

A first ring being “spirofused” with a second ring means the first ring and the second ring share one atom there between.

The terms “halo” or “halogen” as used herein, whether it is used alone or as part of another group, refers to a halogen atom and includes fluoro, chloro, bromo and iodo.

The term “available”, as in “available hydrogen atoms” or “available atoms” refers to atoms that would be known to a person skilled in the art to be capable of replacement by a alternative atom or group of atoms.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3^{rd} Edition, 2003, Georg Thieme Verlag (The Americas).

### II. Compounds and Compositions of the Application

In one aspect, the present application includes a compound of Formula III or a salt and/or tautomer thereof

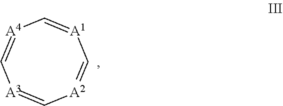

wherein:
A¹, A², A³ and A⁴ are each independently selected from C and N, and 0, 2, or 4 of A¹, A², A³ and A⁴ are N; and
1 to 6 of the carbons of the compound of Formula III are each independently substituted with CN or NH₂.

In some embodiments, the compound of Formula III is selected from

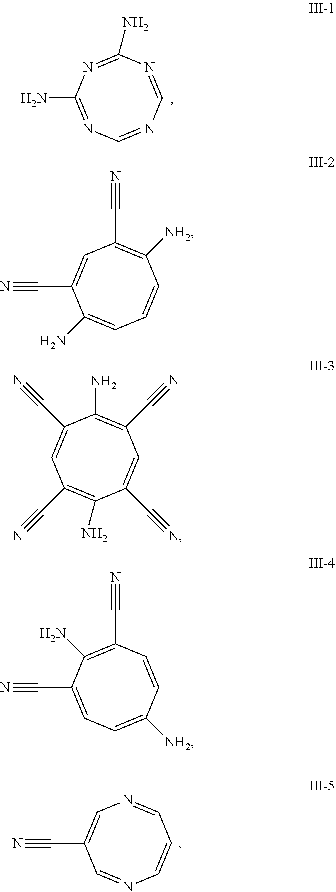

and a salt and/or tautomer thereof.

In another aspect, the present application includes a compound of Formula VII or a salt and/or tautomer thereof

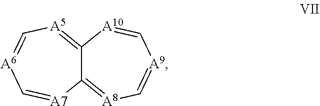

wherein:
A⁵, A⁶, A⁷, A⁸, A⁹ and A¹⁰ are each independently selected from C and N, and 1 to 5 of A⁵, A⁶, A⁷, A⁸, A⁹ and A¹⁰ are N; and
1 to 6 of the carbons of the compound of Formula VII are each independently substituted with CN or NH₂.

In some embodiments, the compound of Formula VII is selected from

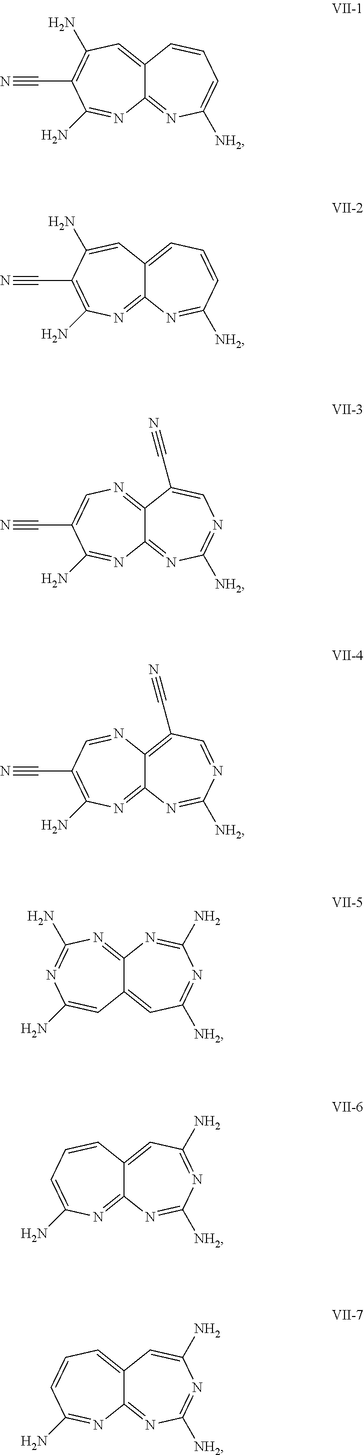

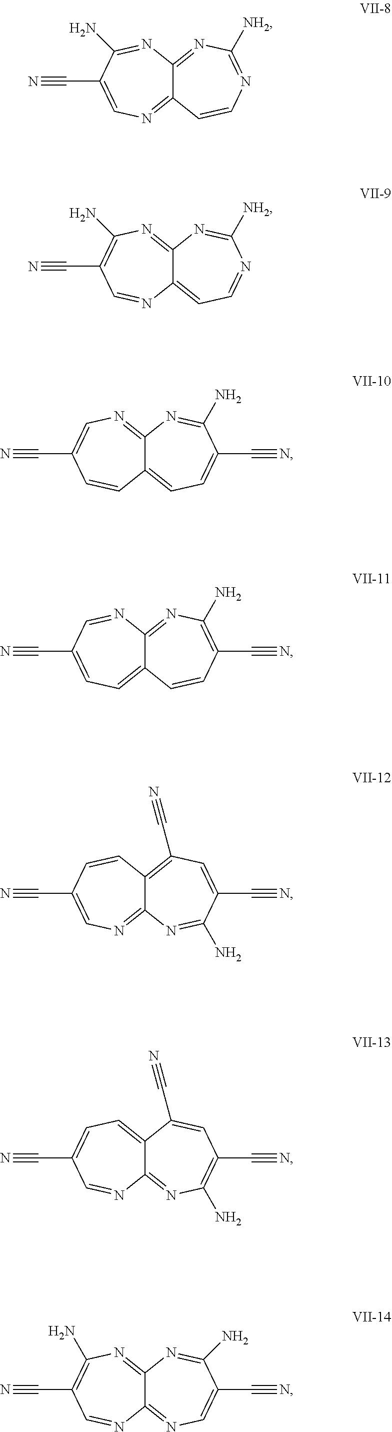

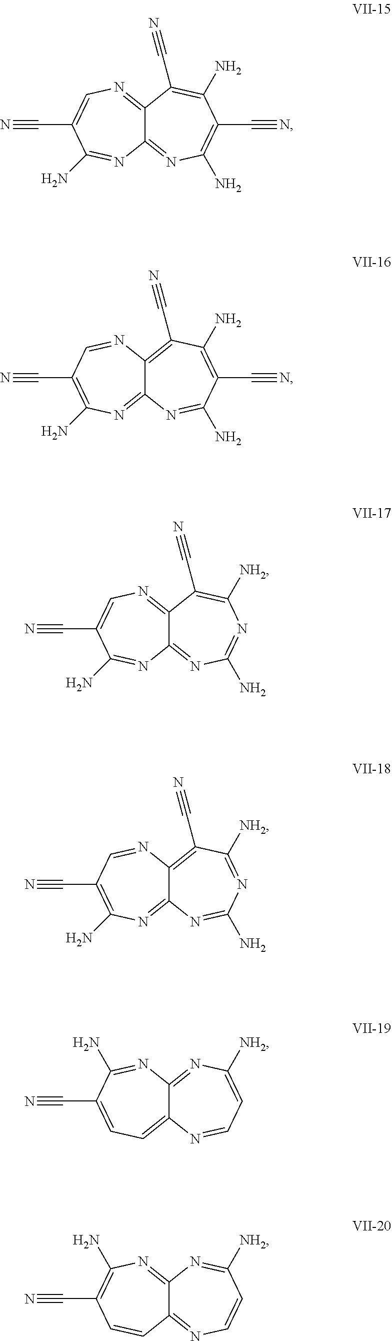 [SMILES: N#CC1=CN=C2C(=NC(N)=C(C#N)C(N)=C2C#N)N=C1N ; N#CC1=C(N)N=C2N=C(N)C(C#N)=C(N)C(C#N)=C2N=C1 ; N#CC1=CN=C2C(=NC(N)=NC(N)=C2C#N)N=C1N ; N#CC1=C(N)N=C2N=C(N)N=C(N)C(C#N)=C2N=C1 ; N#CC1=CC=C2N=CC=C(N)N=C2N=C1N ; N#CC1=C(N)N=C2N=C(N)C=CN=C2C=C1]

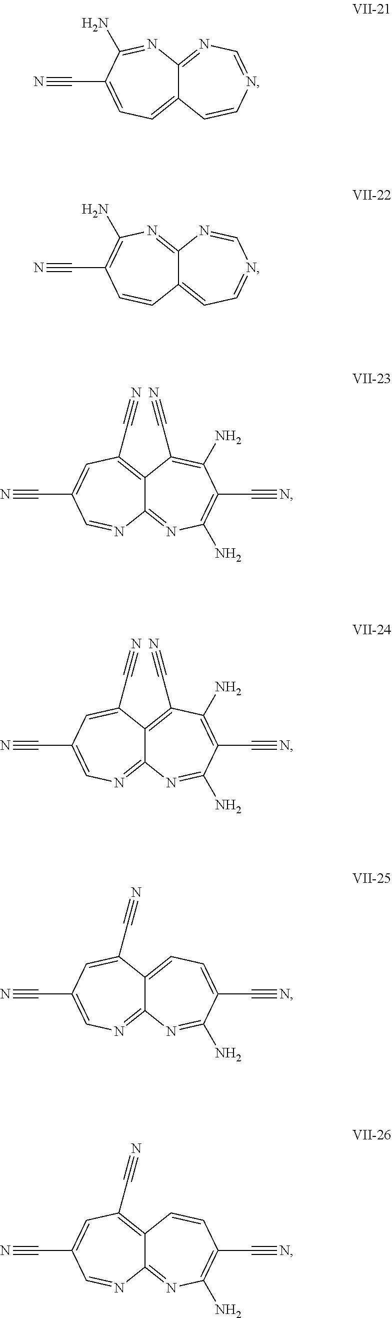 [SMILES: N#CC1=CC=C2C=CN=CN=C2N=C1N ; N#CC1=C(N)N=C2N=CN=CC=C2C=C1 ; N#CC1=CC(C#N)=C2C(=NC(N)=C(C#N)C(N)=C2C#N)N=C1 ; N#CC1=CN=C2N=C(N)C(C#N)=C(N)C(C#N)=C2C(C#N)=C1 ; N#CC1=CN=C2N=C(N)C(C#N)=CC=C2C(C#N)=C1 ; N#CC1=CC(C#N)=C2C=CC(C#N)=C(N)N=C2N=C1]

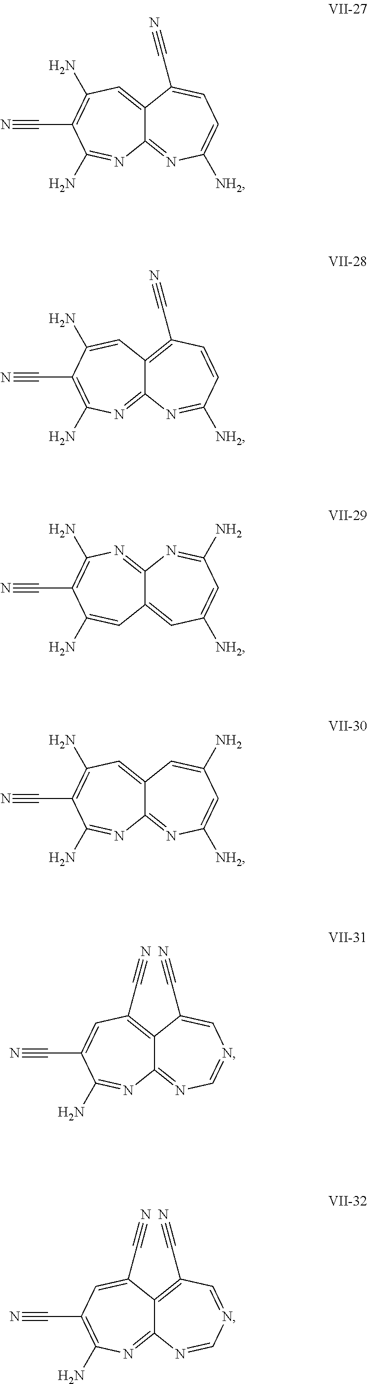 [SMILES: N#CC1=CC=C(N)N=C2N=C(N)C(C#N)=C(N)C=C12 ; N#CC1=C2C=C(N)C(C#N)=C(N)N=C2N=C(N)C=C1 ; N#CC1=C(N)N=C2N=C(N)C=C(N)C=C2C=C1N ; N#CC1=C(N)C=C2C=C(N)C=C(N)N=C2N=C1N ; N#CC1=CC(C#N)=C2C(C#N)=CN=CN=C2N=C1N ; N#CC1=CC(C#N)=C(N)N=C2N=CN=CC(C#N)=C12]

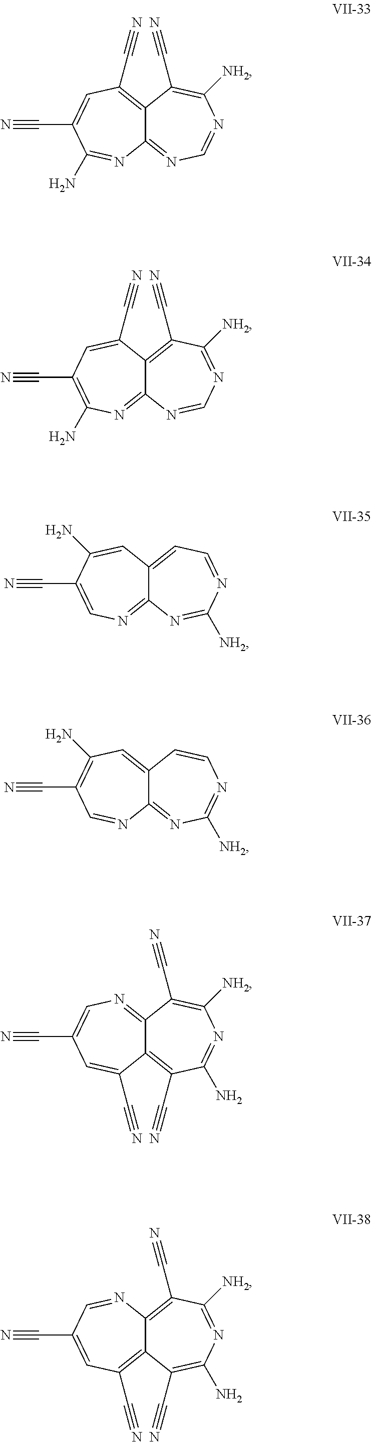 [SMILES: N#CC1=CC(C#N)=C2C(=NC=NC(N)=C2C#N)N=C1N ; N#CC1=CC(C#N)=C(N)N=C2N=CN=C(N)C(C#N)=C12 ; N#CC1=CN=C2N=C(N)N=CC=C2C=C1N ; N#CC1=C(N)C=C2C=CN=C(N)N=C2N=C1 ; N#CC1=CN=C2C(C#N)=C(N)N=C(N)C(C#N)=C2C(C#N)=C1 ; N#CC1=CC(C#N)=C2C(C#N)=C(N)N=C(N)C(C#N)=C2N=C1]

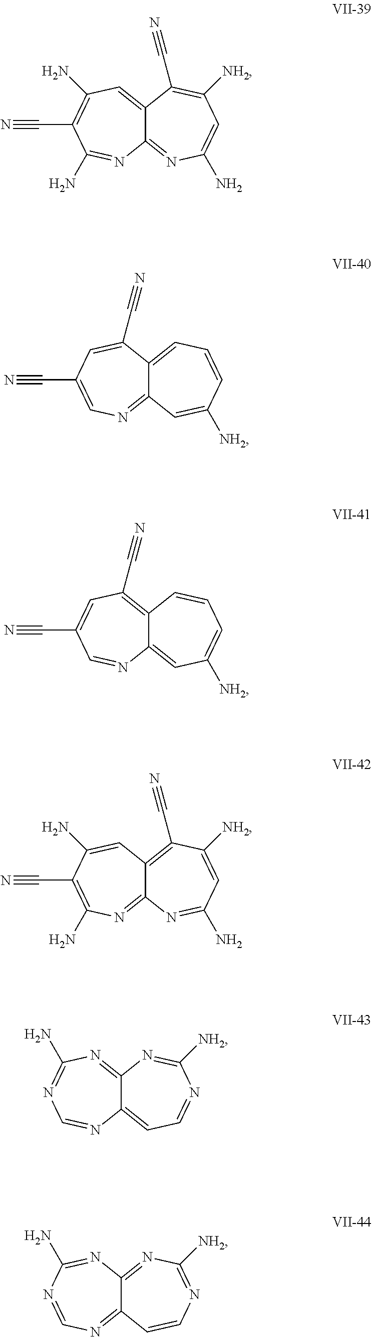 [SMILES: N#CC1=C(N)C=C(N)N=C2N=C(N)C(C#N)=C(N)C=C21 ; N#CC1=CN=C2C=C(N)C=CC=C2C(C#N)=C1 ; N#CC1=CC(C#N)=C2C=CC=C(N)C=C2N=C1 ; N#CC1=C(N)N=C2N=C(N)C=C(N)C(C#N)=C2C=C1N ; NC1=NC=NC2=CC=NC(N)=NC2=N1 ; NC1=NC=CC2=NC=NC(N)=NC2=N1]

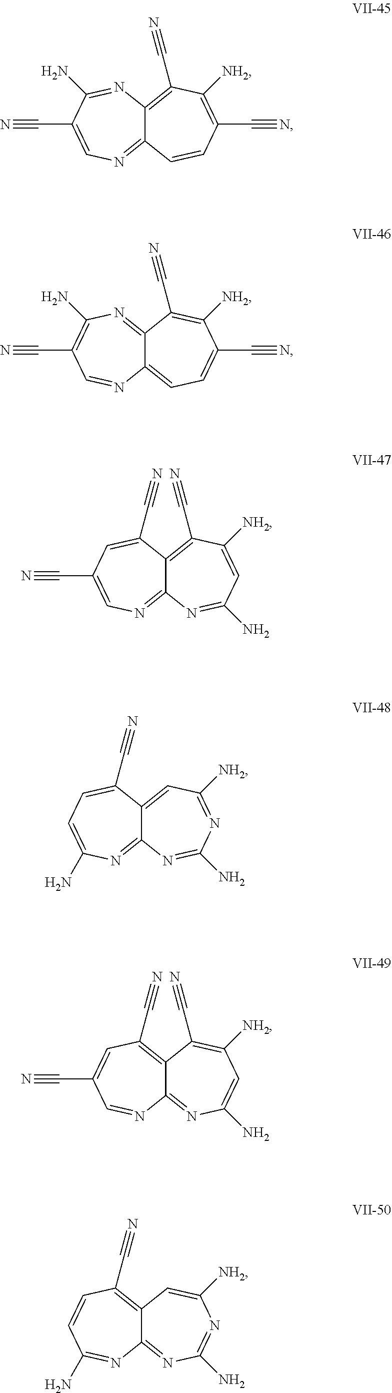 [SMILES: N#CC1=CN=C2C=CC(C#N)=C(N)C(C#N)=C2N=C1N ; N#CC1=CC=C2N=CC(C#N)=C(N)N=C2C(C#N)=C1N ; N#CC1=CN=C2N=C(N)C=C(N)C(C#N)=C2C(C#N)=C1 ; N#CC1=CC=C(N)N=C2N=C(N)N=C(N)C=C12 ; N#CC1=CC(C#N)=C2C(=NC(N)=CC(N)=C2C#N)N=C1 ; N#CC1=C2C=C(N)N=C(N)N=C2N=C(N)C=C1]

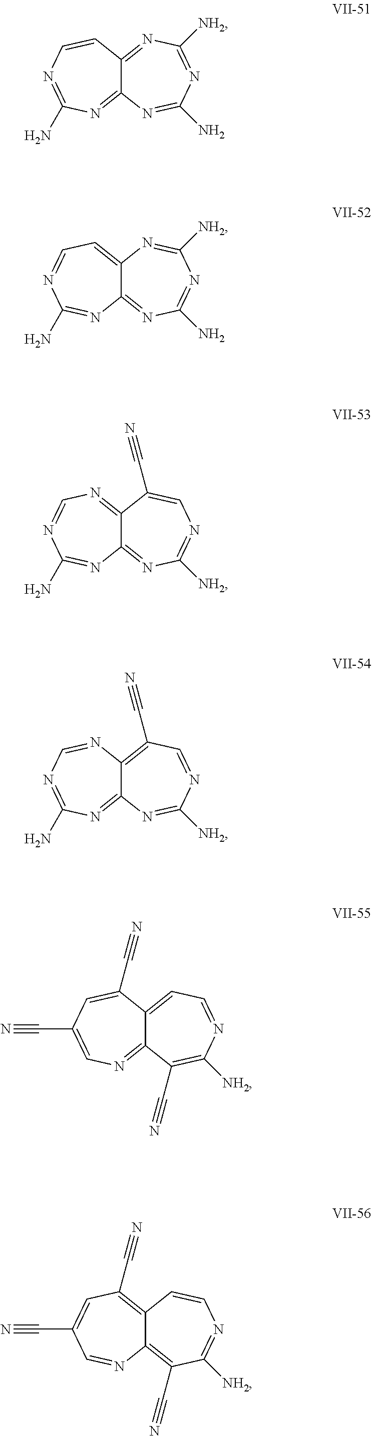 [SMILES: NC1=NC=CC2=NC(N)=NC(N)=NC2=N1 ; NC1=NC2=CC=NC(N)=NC2=NC(N)=N1 ; N#CC1=CN=C(N)N=C2N=C(N)N=CN=C12 ; N#CC1=C2N=CN=C(N)N=C2N=C(N)N=C1 ; N#CC1=CN=C2C(=CC=NC(N)=C2C#N)C(C#N)=C1 ; N#CC1=CC(C#N)=C2C=CN=C(N)C(C#N)=C2N=C1]

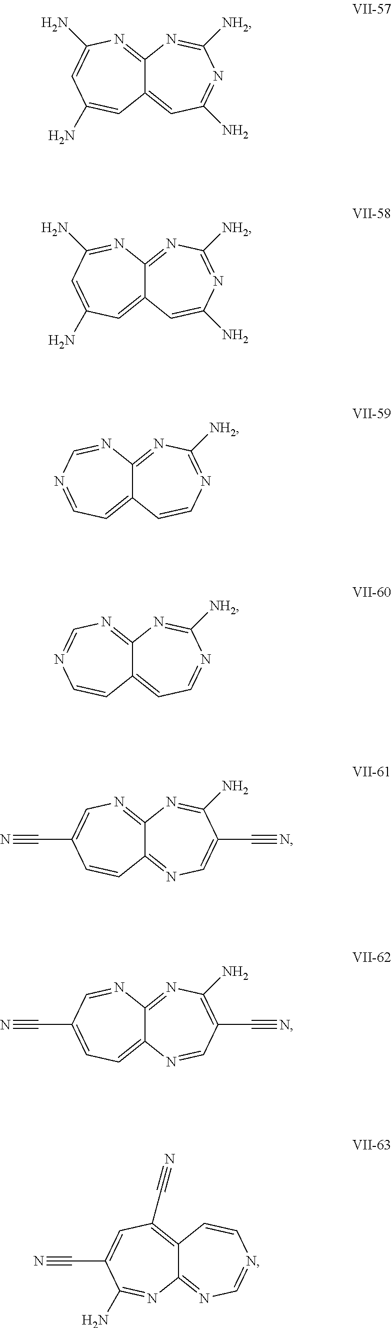

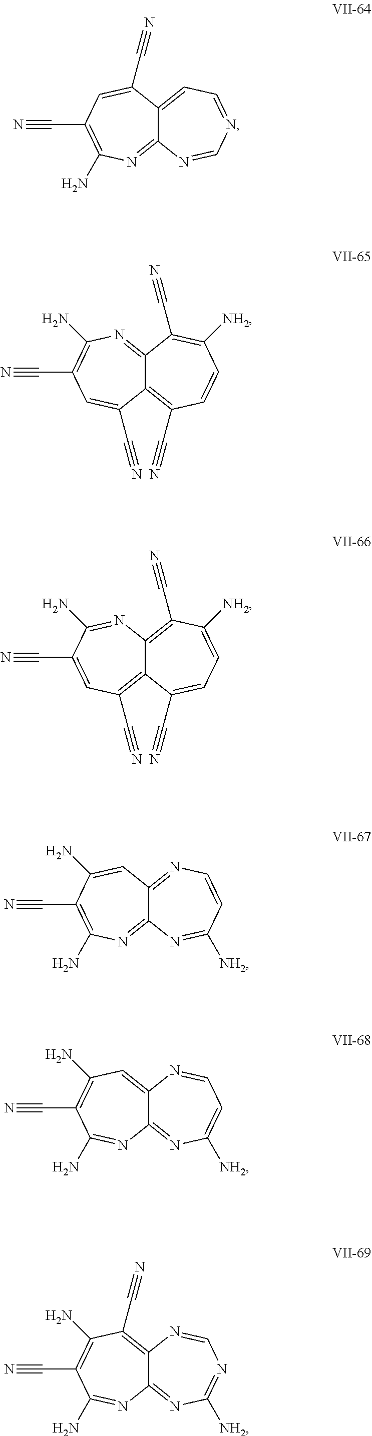 [SMILES: N#CC1=CC(C#N)=C(N)N=C2N=CN=CC=C12 ; N#CC1=CC(C#N)=C(N)N=C2C(C#N)=C(N)C=CC(C#N)=C12 ; N#CC1=CC(C#N)=C2C(C#N)=CC=C(N)C(C#N)=C2N=C1N ; N#CC1=C(N)N=C2N=C(N)C=CN=C2C=C1N ; N#CC1=C(N)C=C2N=CC=C(N)N=C2N=C1N ; N#CC1=C(N)C(C#N)=C2N=CN=C(N)N=C2N=C1N]

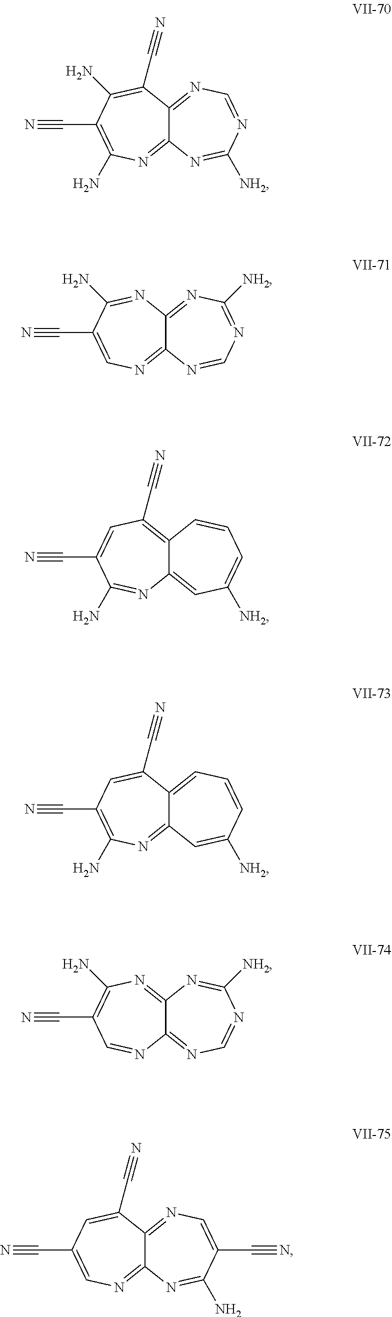 [SMILES: N#CC1=C(N)N=C2N=C(N)N=CN=C2C(C#N)=C1N ; N#CC1=CN=C2N=CN=C(N)N=C2N=C1N ; N#CC1=CC(C#N)=C2C=CC=C(N)C=C2N=C1N ; N#CC1=CC(C#N)=C(N)N=C2C=C(N)C=CC=C12 ; N#CC1=C(N)N=C2N=C(N)N=CN=C2N=C1 ; N#CC1=CN=C2N=C(N)C(C#N)=CN=C2C(C#N)=C1]

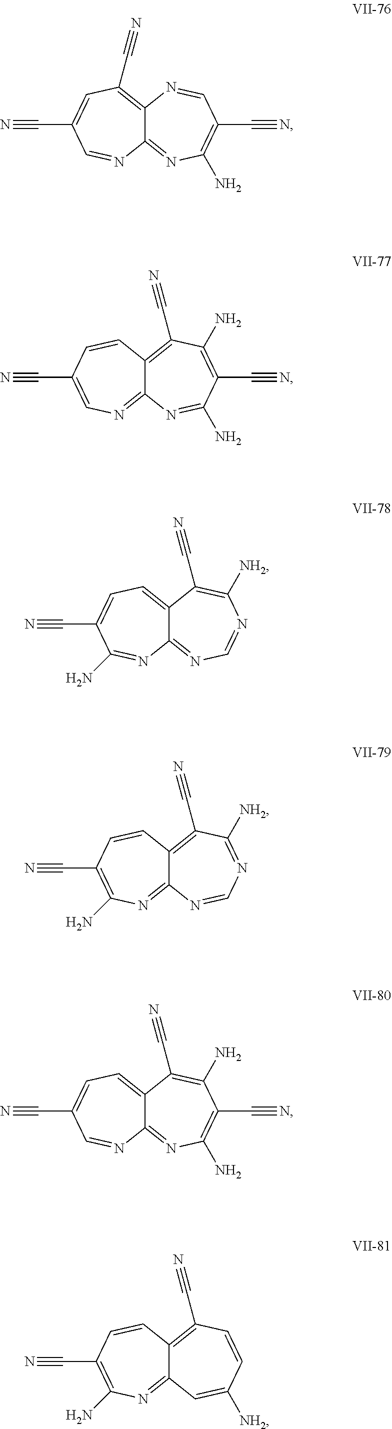 [SMILES: N#CC1=CC(C#N)=C2N=CC(C#N)=C(N)N=C2N=C1 ; N#CC1=CN=C2N=C(N)C(C#N)=C(N)C(C#N)=C2C=C1 ; N#CC1=CC=C2C(=NC=NC(N)=C2C#N)N=C1N ; N#CC1=C(N)N=C2N=CN=C(N)C(C#N)=C2C=C1 ; N#CC1=CC=C2C(=NC(N)=C(C#N)C(N)=C2C#N)N=C1 ; N#CC1=C(N)N=C2C=C(N)C=CC(C#N)=C2C=C1]

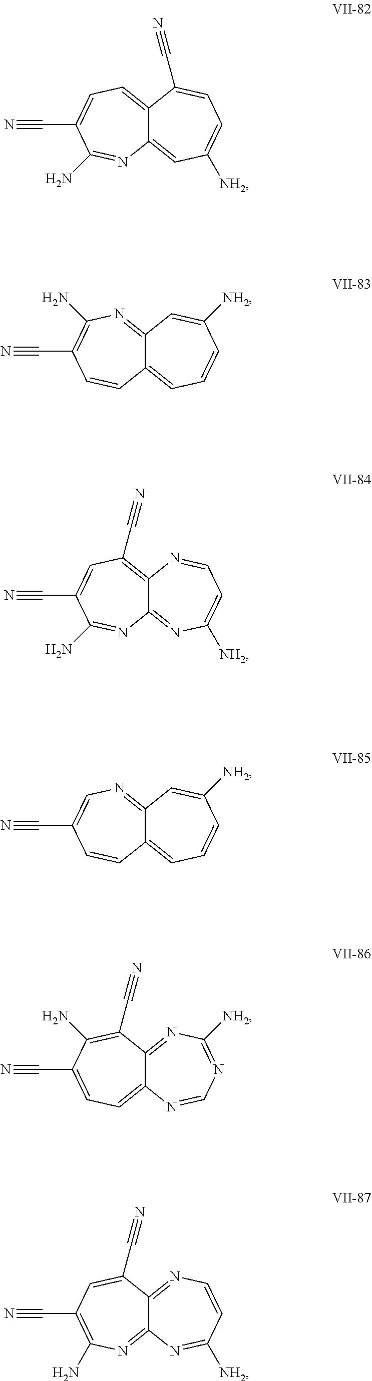 [SMILES: N#CC1=CC=C(N)C=C2N=C(N)C(C#N)=CC=C12 ; N#CC1=C(N)N=C2C=C(N)C=CC=C2C=C1 ; N#CC1=CC(C#N)=C2N=CC=C(N)N=C2N=C1N ; N#CC1=CN=C2C=C(N)C=CC=C2C=C1 ; N#CC1=CC=C2N=CN=C(N)N=C2C(C#N)=C1N ; N#CC1=CC(C#N)=C(N)N=C2N=C(N)C=CN=C12]

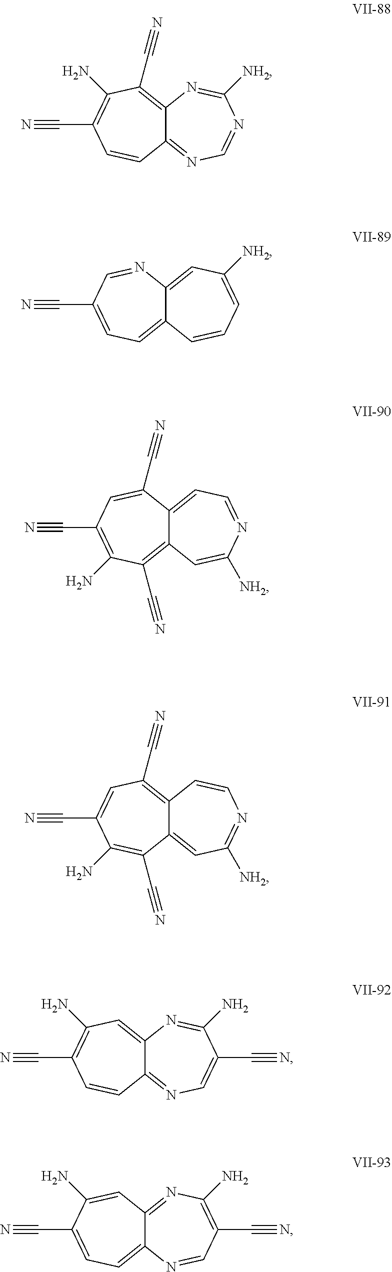 [SMILES: N#CC1=C(N)C(C#N)=C2N=C(N)N=CN=C2C=C1 ; N#CC1=CC=C2C=CC=C(N)C=C2N=C1 ; N#CC1=CC(C#N)=C(N)C(C#N)=C2C=C(N)N=CC=C12 ; N#CC1=CC(C#N)=C2C=CN=C(N)C=C2C(C#N)=C1N ; N#CC1=CN=C2C=CC(C#N)=C(N)C=C2N=C1N ; N#CC1=CC=C2N=CC(C#N)=C(N)N=C2C=C1N]

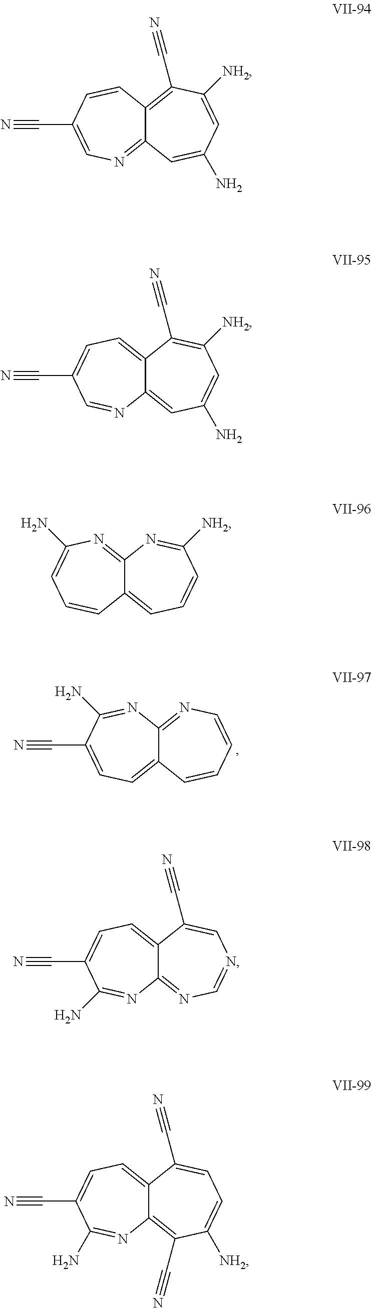 [SMILES: N#CC1=CN=C2C=C(N)C=C(N)C(C#N)=C2C=C1 ; N#CC1=CC=C2C(=CC(N)=CC(N)=C2C#N)N=C1 ; NC1=CC=CC2=CC=CC(N)=NC2=N1 ; N#CC1=CC=C2C=CC=CN=C2N=C1N ; N#CC1=CN=CN=C2N=C(N)C(C#N)=CC=C12 ; N#CC1=CC=C(N)C(C#N)=C2N=C(N)C(C#N)=CC=C12]

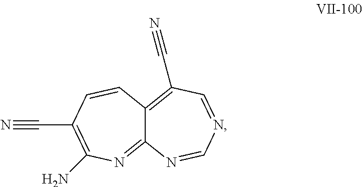 [SMILES: N#CC1=C(N)N=C2N=CN=CC(C#N)=C2C=C1]

and a salt and/or tautomer thereof.

In another aspect, the present application includes a compound of Formula IX or a salt and/or tautomer thereof

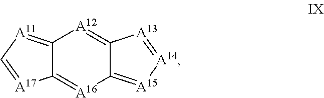

wherein:
A¹¹, A¹², A¹³, A¹⁴, A¹⁵, A¹⁶ and A¹⁷ are each independently selected from C and N, and 0 to 6 of A¹¹, A¹², A¹³, A¹⁴, A¹⁵, A¹⁶ and A¹⁷ are N; and
0 to 6 of the carbons of the compound of Formula IX are each independently substituted with CN or NH₂.

In some embodiments, the compound of Formula IX is selected from

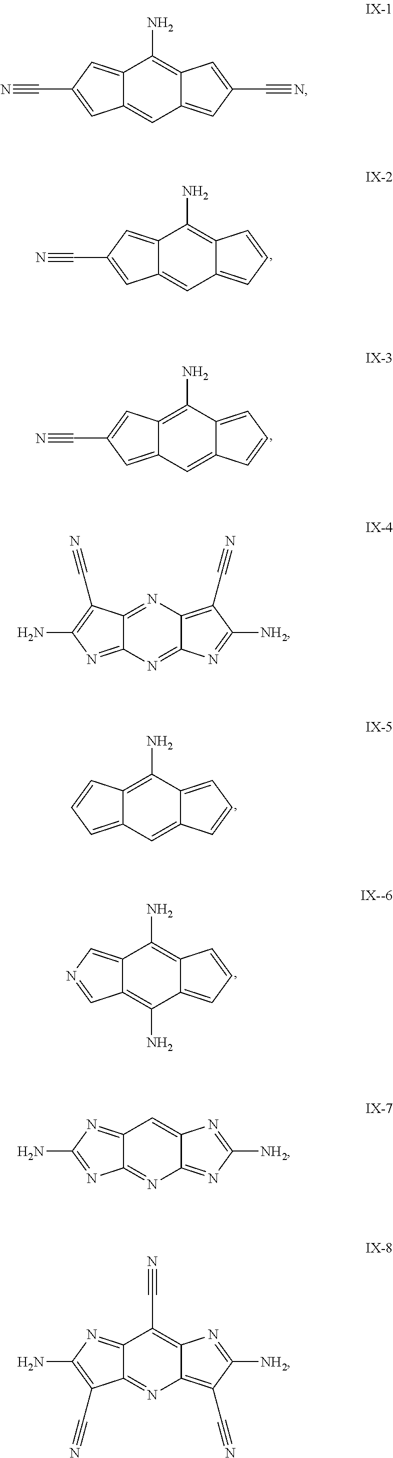

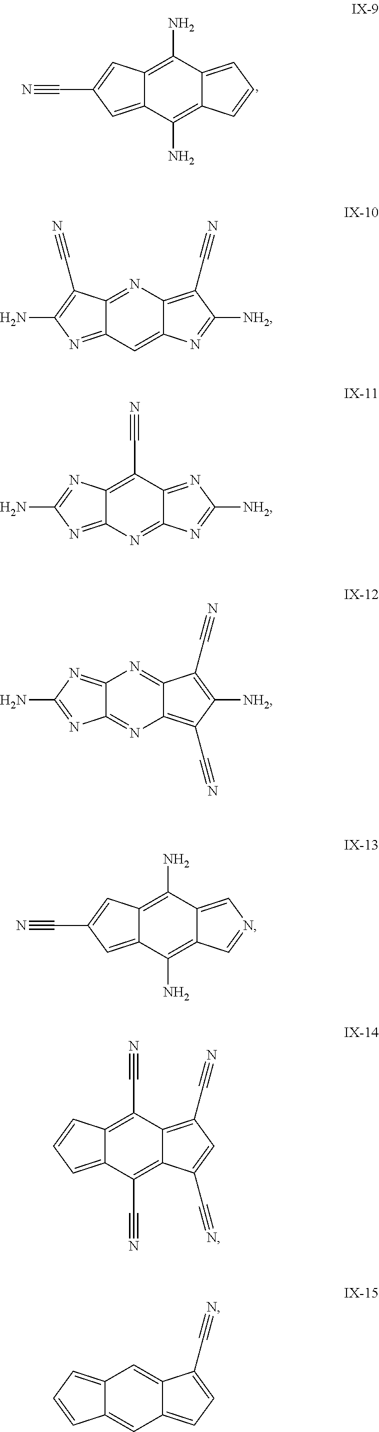

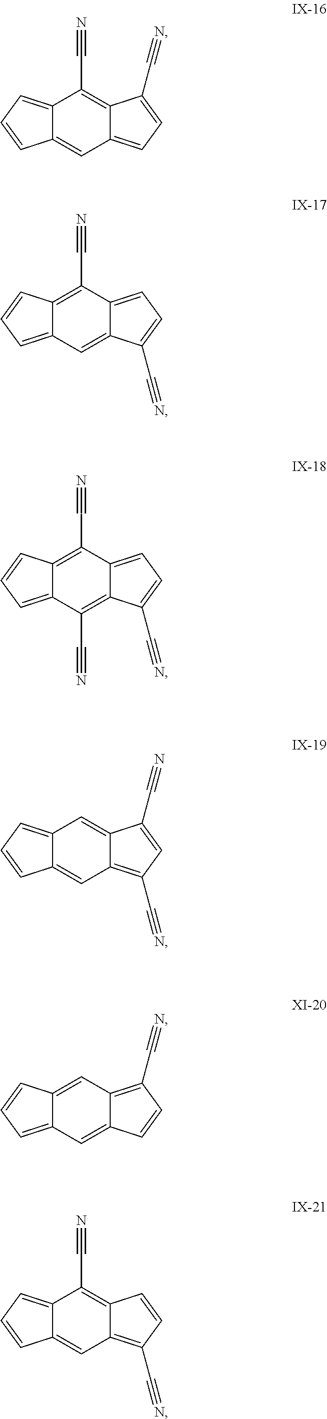 [SMILES: N#CC1=CC=c2cc3c(c(C#N)c21)=CC=C3 ; N#CC1=CC=c2c1cc1c(c2C#N)C=CC=1 ; N#CC1=CC=c2c(C#N)c3c(c(C#N)c21)=CC=C3 ; N#CC1=CC(C#N)=c2cc3c(cc21)=CC=C3 ; N#CC1=c2cc3c(cc2C=C1)=CC=C3 ; N#CC1=c2cc3c(c(C#N)c2C=C1)=CC=C3]

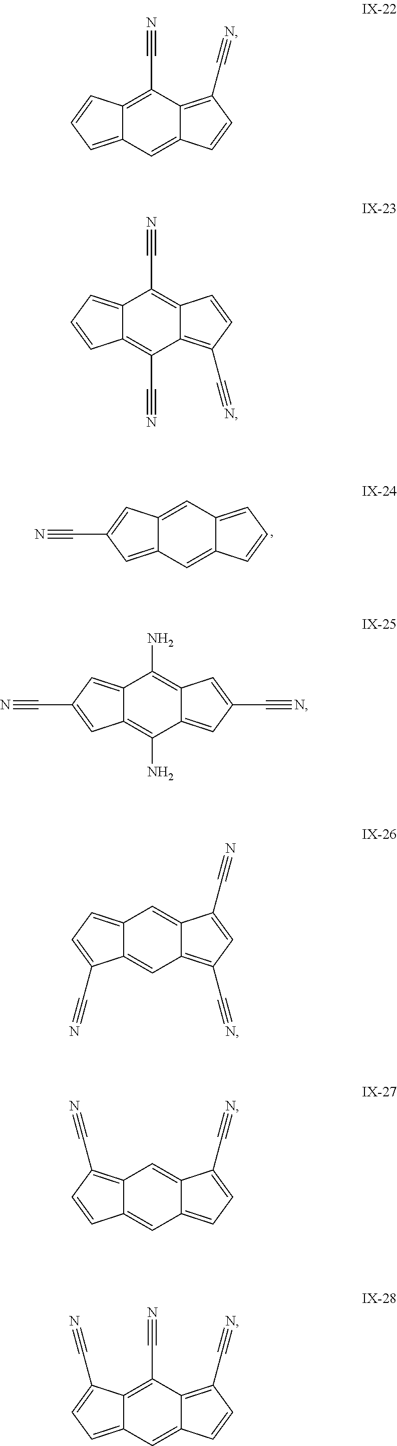

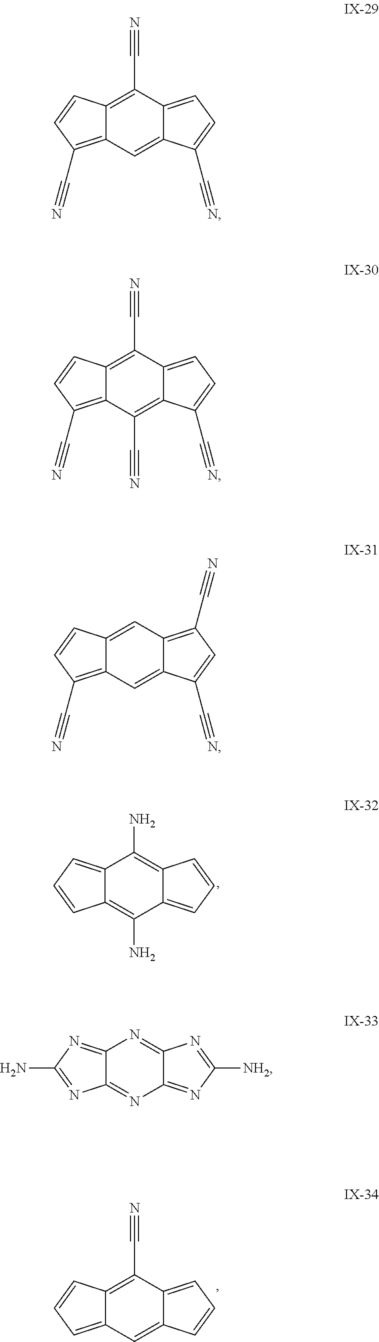 [SMILES: N#CC1=CC=c2c1cc1c(c2C#N)C=CC=1C#N ; N#CC1=CC=c2c(C#N)c3c(c(C#N)c21)=C(C#N)C=C3 ; N#CC1=CC(C#N)=c2cc3c(cc21)=C(C#N)C=C3 ; Nc1c2c(c(N)c3c1=CC=C3)=CC=C2 ; NC1=Nc2nc3c(nc2=N1)N=C(N)N=3 ; N#Cc1c2c(cc3c1=CC=C3)=CC=C2]

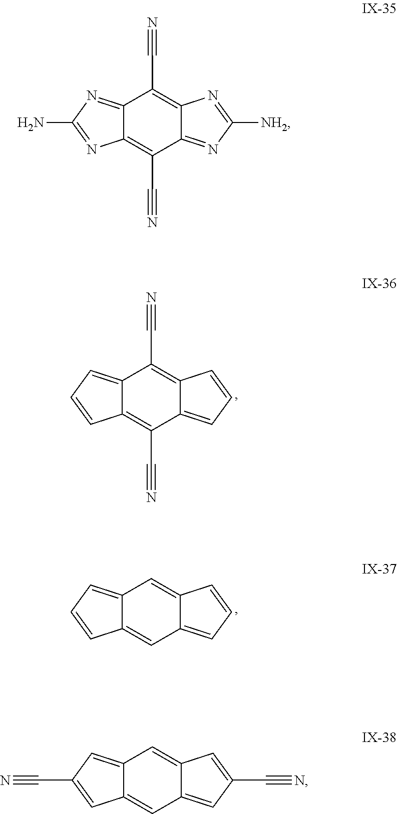 [SMILES: N#Cc1c2c(c(C#N)c3c1=NC(N)=N3)=NC(N)=N2 ; N#Cc1c2c(c(C#N)c3c1=CC=C3)=CC=C2 ; C1=Cc2cc3c(cc2=C1)C=CC=3 ; N#CC1=Cc2cc3c(cc2=C1)C=C(C#N)C=3]

and a salt and/or tautomer thereof.

In another aspect, the present application includes a compound of Formula XII or a salt and/or tautomer thereof

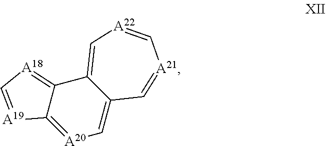

wherein:
A¹⁸, A¹⁹, A²⁰, A²¹ and A²² are each independently selected from C and N, and 0 to 5 of A¹⁸, A¹⁹, A²⁰, A²¹ and A²² are N; and
0 to 6 carbons of the compound of Formula XII are each independently substituted with CN or NH₂.

In some embodiments, the compound of Formula XII is selected from,

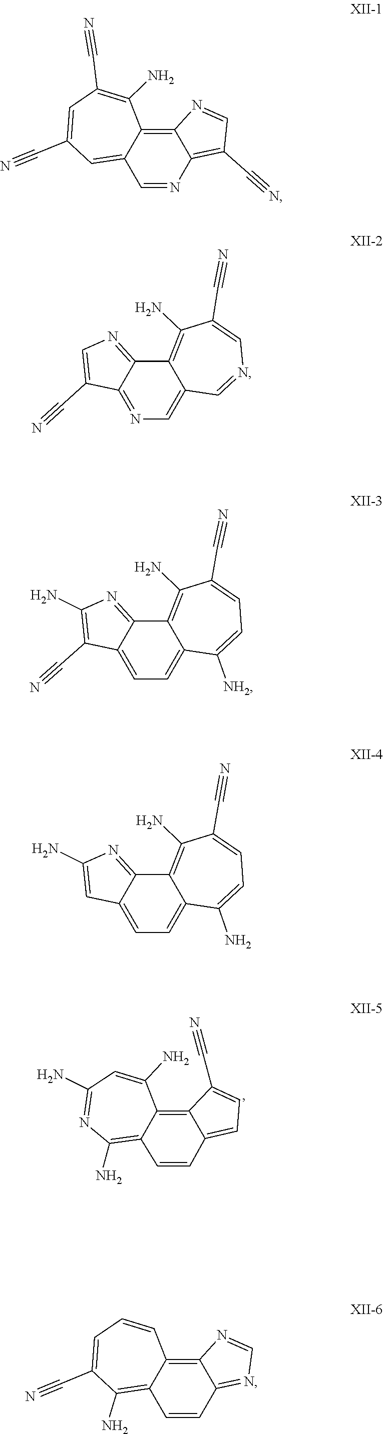 [SMILES: N#Cc1cc(C#N)c(N)c2c(cnc3c(C#N)cnc32)c1 ; N#Cc1cncc2cnc3c(C#N)cnc3c2c1N ; N#Cc1ccc(N)c2ccc3c(C#N)c(N)nc3c2c1N ; N#Cc1ccc(N)c2ccc3cc(N)nc3c2c1N ; N#Cc1ccc2ccc3c(N)nc(N)cc(N)c3c12 ; N#Cc1cccc2c(ccc3ncnc32)c1N]

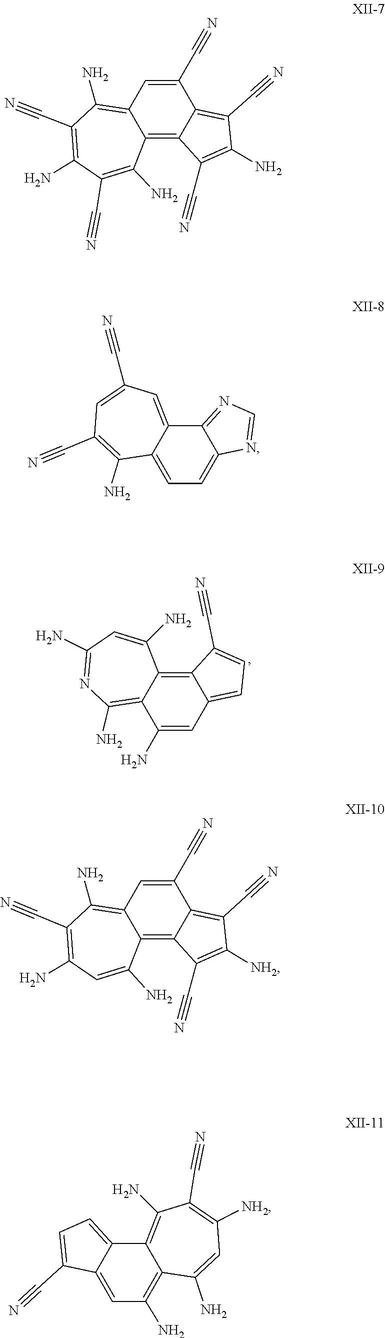 [SMILES: N#Cc1c(N)c(C#N)c(N)c2c(cc(C#N)c3c(C#N)c(N)c(C#N)c32)c1N ; N#Cc1cc(C#N)c(N)c2ccc3ncnc3c2c1 ; N#Cc1ccc2cc(N)c3c(N)nc(N)cc(N)c3c12 ; N#Cc1c(N)cc(N)c2c(cc(C#N)c3c(C#N)c(N)c(C#N)c32)c1N ; N#Cc1c(N)cc(N)c2c(N)cc3c(C#N)ccc3c2c1N]

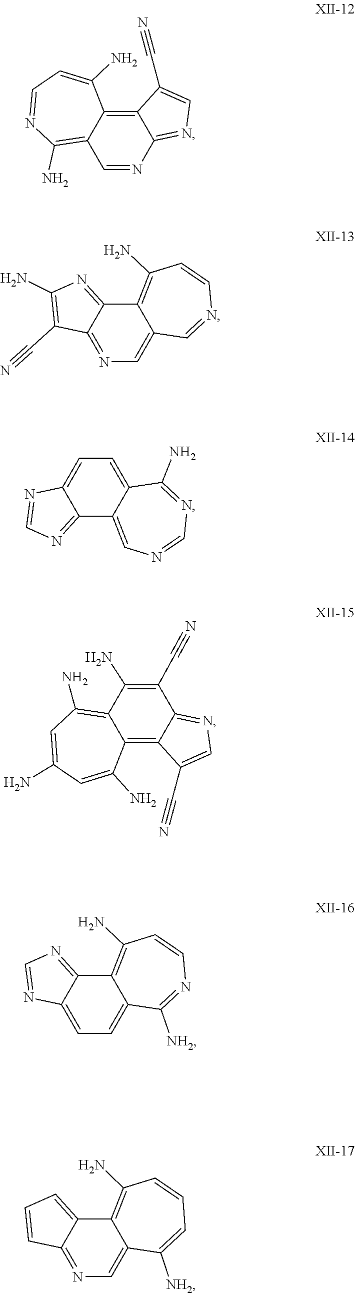 [SMILES: N#Cc1cnc2ncc3c(N)nccc(N)c3c12 ; N#Cc1c(N)nc2c1ncc1cnccc(N)c12 ; Nc1ncncc2c1ccc1ncnc12 ; N#Cc1c(N)c2c(N)cc(N)cc(N)c2c2c(C#N)cnc12 ; Nc1nccc(N)c2c1ccc1ncnc12 ; Nc1cccc(N)c2c1cnc1cccc12]

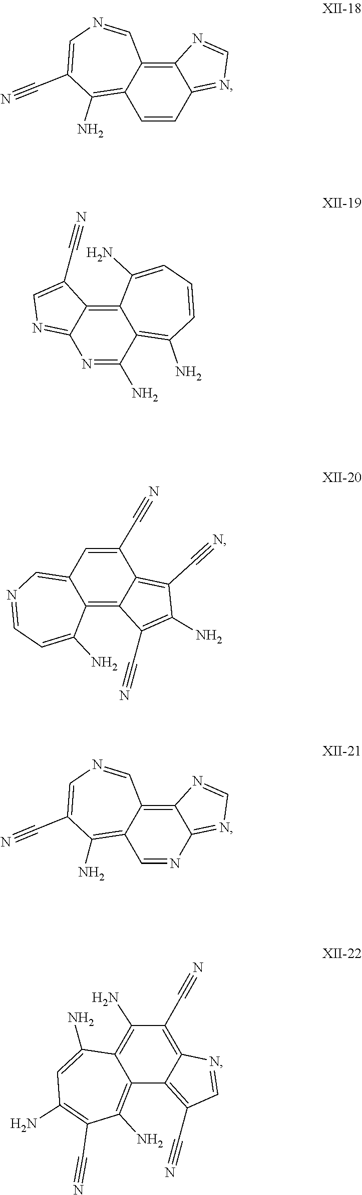 [SMILES: N#Cc1cncc2c(ccc3ncnc32)c1N ; N#Cc1cnc2nc(N)c3c(N)cccc(N)c3c12 ; N#Cc1cc2cnccc(N)c2c2c(C#N)c(N)c(C#N)c12 ; N#Cc1cncc2c(cnc3ncnc32)c1N ; N#Cc1c(N)cc(N)c2c(N)c(C#N)c3ncc(C#N)c3c2c1N]

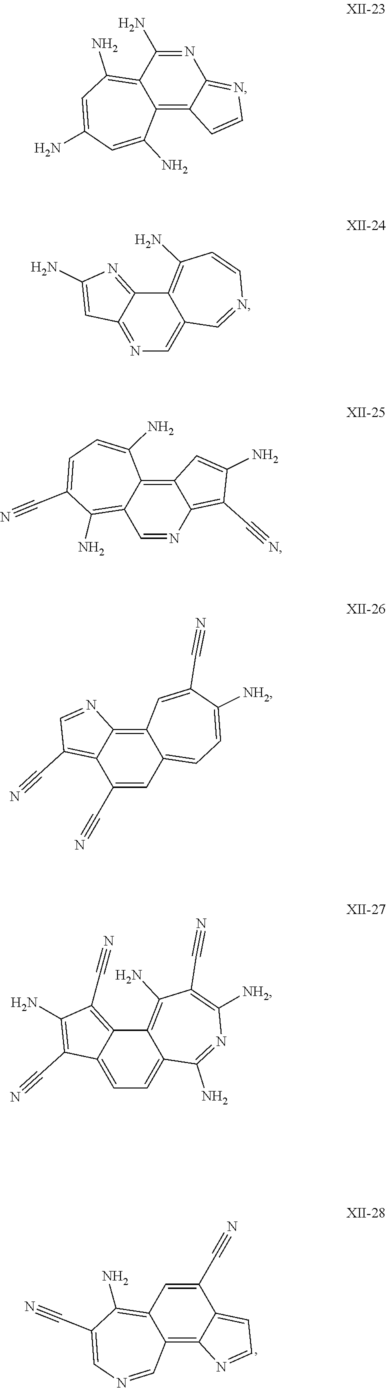 [SMILES: Nc1cc(N)c2c(N)nc3nccc3c2c(N)c1 ; Nc1cc2ncc3cnccc(N)c3c2n1 ; N#Cc1ccc(N)c2c(cnc3c(C#N)c(N)cc32)c1N ; N#Cc1cc2c(ccc1N)cc(C#N)c1c(C#N)cnc12 ; N#Cc1c(N)nc(N)c2ccc3c(C#N)c(N)c(C#N)c3c2c1N ; N#Cc1cncc2c(cc(C#N)c3ccnc32)c1N]

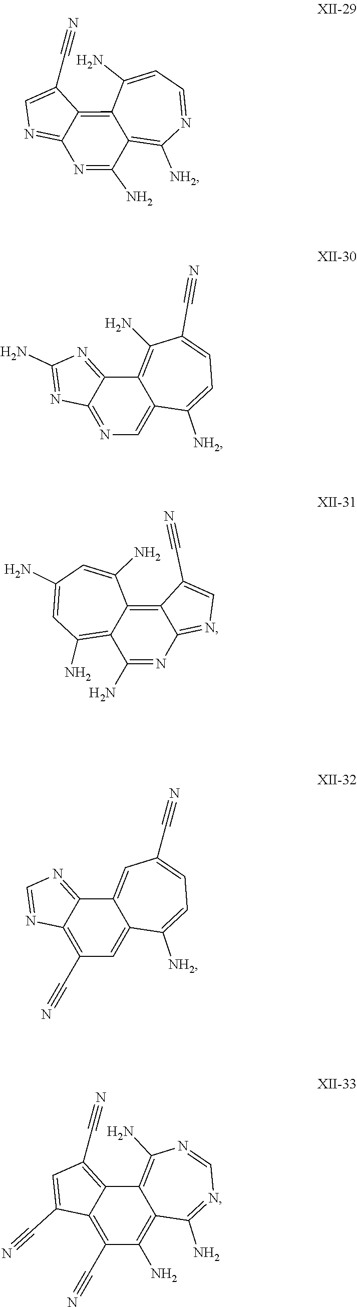 [SMILES: N#Cc1cnc2nc(N)c3c(N)nccc(N)c3c12 ; N#Cc1ccc(N)c2cnc3nc(N)nc3c2c1N ; N#Cc1cnc2nc(N)c3c(N)cc(N)cc(N)c3c12 ; N#Cc1ccc(N)c2cc(C#N)c3ncnc3c2c1 ; N#Cc1cc(C#N)c2c1c(C#N)c(N)c1c(N)ncnc(N)c12]

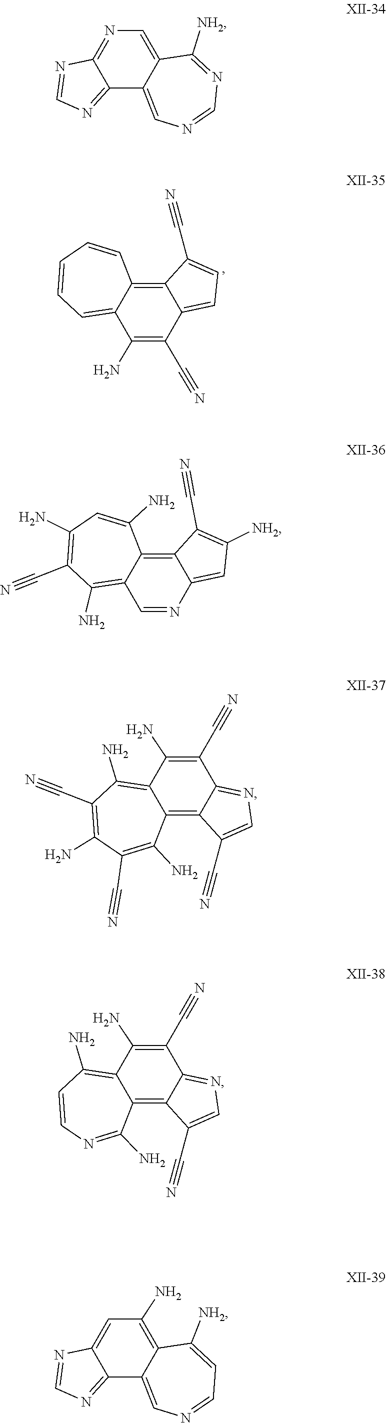 [SMILES: Nc1ncncc2c1cnc1ncnc12 ; N#Cc1c(N)c2cccccc2c2c(C#N)ccc12 ; N#Cc1c(N)cc(N)c2c(cnc3cc(N)c(C#N)c32)c1N ; N#Cc1c(N)c(C#N)c(N)c2c(c1N)c(N)c(C#N)c1ncc(C#N)c12 ; N#Cc1c(N)c2c(N)ccnc(N)c2c2c(C#N)cnc12 ; Nc1ccncc2c1c(N)cc1ncnc12]

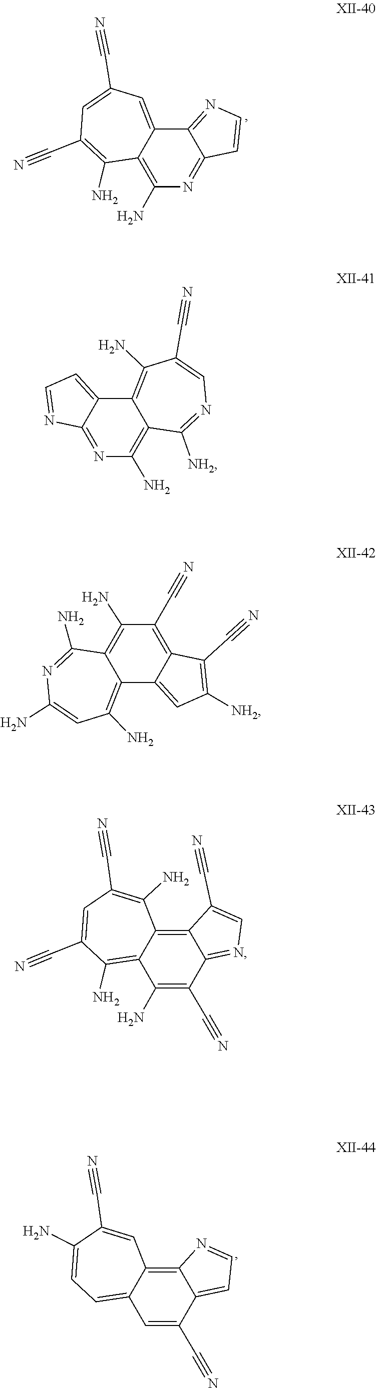 [SMILES: N#Cc1cc(C#N)c(N)c2c(N)nc3ccnc3c2c1 ; N#Cc1cnc(N)c2c(N)nc3nccc3c2c1N ; N#Cc1c(N)cc2c1c(C#N)c(N)c1c(N)nc(N)cc(N)c12 ; N#Cc1cc(C#N)c(N)c2c(c1N)c(N)c(C#N)c1ncc(C#N)c12 ; N#Cc1cc2c(ccc1N)cc(C#N)c1ccnc12]

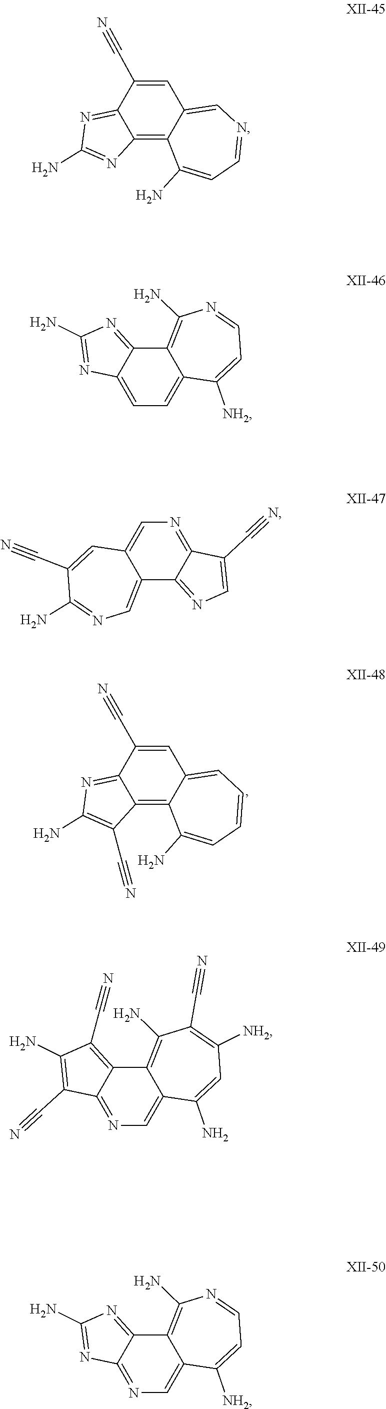 [SMILES: N#Cc1cc2cnccc(N)c2c2nc(N)nc12 ; Nc1nc2ccc3c(N)ccnc(N)c3c2n1 ; N#Cc1cc2cnc3c(C#N)cnc3c2cnc1N ; N#Cc1cc2ccccc(N)c2c2c(C#N)c(N)nc12 ; N#Cc1c(N)cc(N)c2cnc3c(C#N)c(N)c(C#N)c3c2c1N ; Nc1nc2ncc3c(N)ccnc(N)c3c2n1]

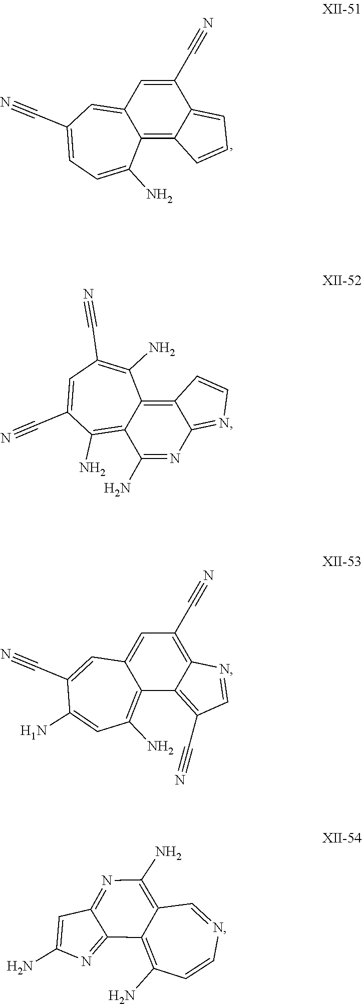

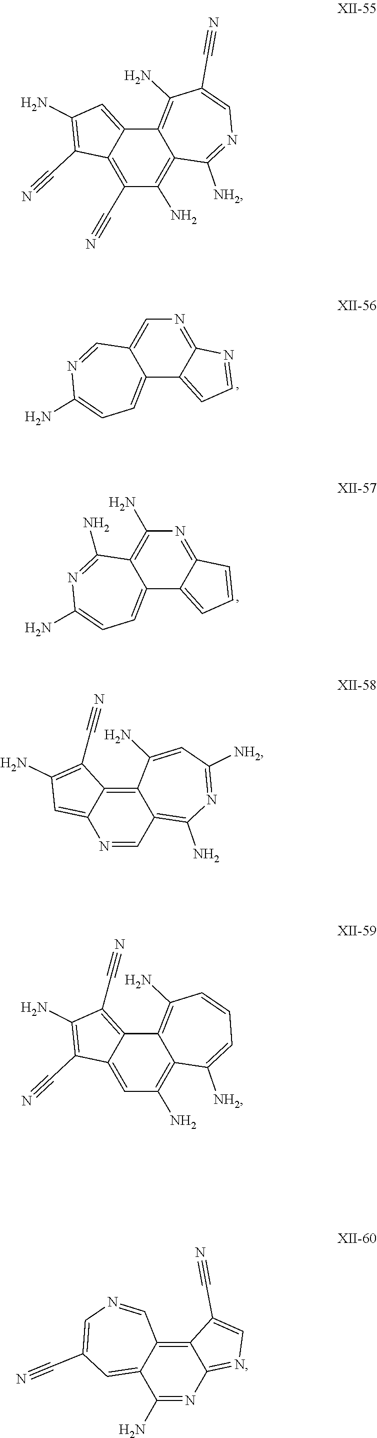 [SMILES: N#Cc1cnc(N)c2c(N)c(C#N)c3c(C#N)c(N)cc3c2c1N ; Nc1ccc2c(cn1)cnc1nccc12 ; Nc1ccc2c(c(N)n1)c(N)nc1cccc12 ; N#Cc1c(N)cc2ncc3c(N)nc(N)cc(N)c3c12 ; N#Cc1c(N)c(C#N)c2c1cc(N)c1c(N)cccc(N)c12 ; N#Cc1cncc2c(c1)c(N)nc1ncc(C#N)c12]

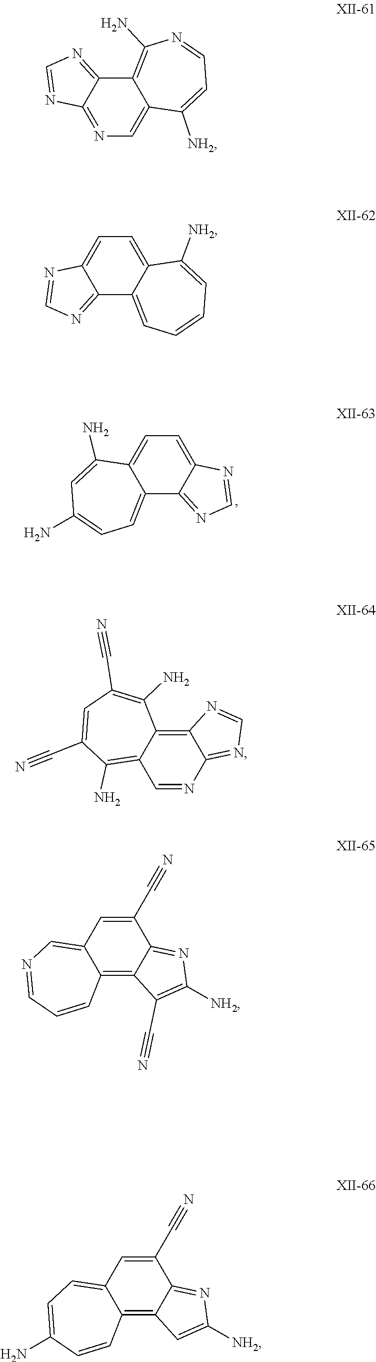 [SMILES: Nc1ccnc(N)c2c1cnc1ncnc12 ; Nc1ccccc2c1ccc1ncnc12 ; Nc1ccc2c(ccc3ncnc32)c(N)c1 ; N#Cc1cc(C#N)c(N)c2c(cnc3ncnc32)c1N ; N#Cc1cc2cncccc2c2c(C#N)c(N)nc12 ; N#Cc1cc2ccc(N)ccc2c2cc(N)nc12]

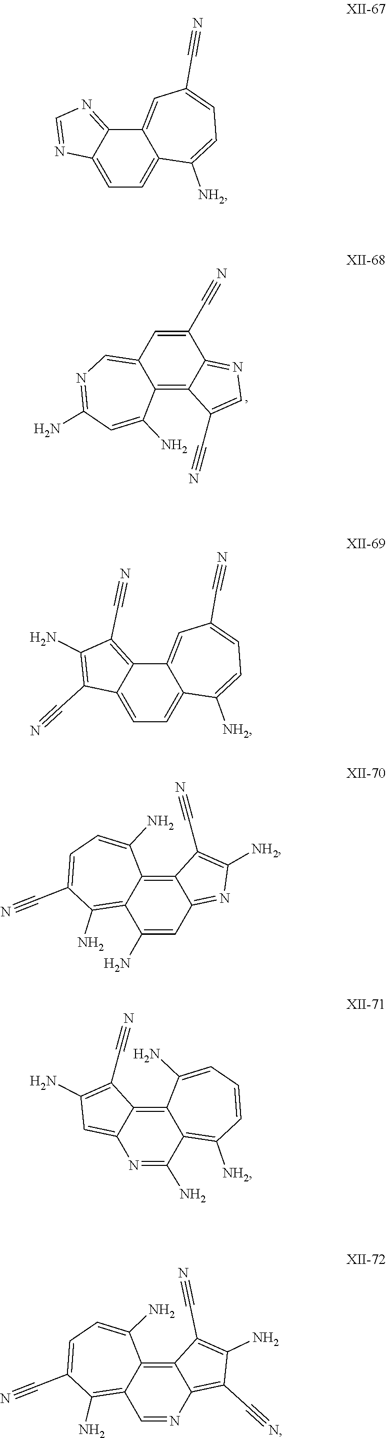 [SMILES: N#Cc1ccc(N)c2ccc3ncnc3c2c1 ; N#Cc1cc2cnc(N)cc(N)c2c2c(C#N)cnc12 ; N#Cc1ccc(N)c2ccc3c(C#N)c(N)c(C#N)c3c2c1 ; N#Cc1ccc(N)c2c(c(N)cc3nc(N)c(C#N)c32)c1N ; N#Cc1c(N)cc2nc(N)c3c(N)cccc(N)c3c12 ; N#Cc1ccc(N)c2c(cnc3c(C#N)c(N)c(C#N)c32)c1N]

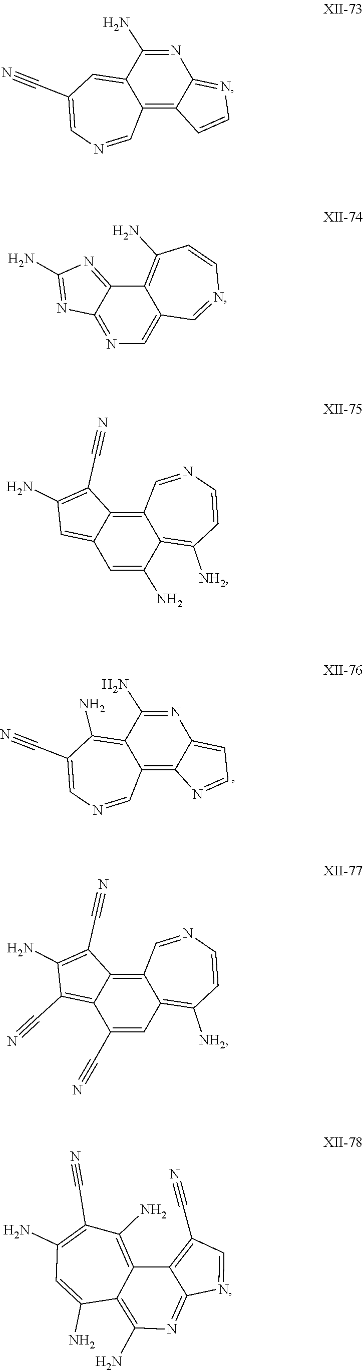 [SMILES: N#Cc1cncc2c(c1)c(N)nc1nccc12 ; Nc1nc2ncc3cnccc(N)c3c2n1 ; N#Cc1c(N)cc2cc(N)c3c(N)ccncc3c12 ; N#Cc1cncc2c(c(N)nc3ccnc32)c1N ; N#Cc1cc2c(N)ccncc2c2c(C#N)c(N)c(C#N)c12 ; N#Cc1c(N)cc(N)c2c(N)nc3ncc(C#N)c3c2c1N]

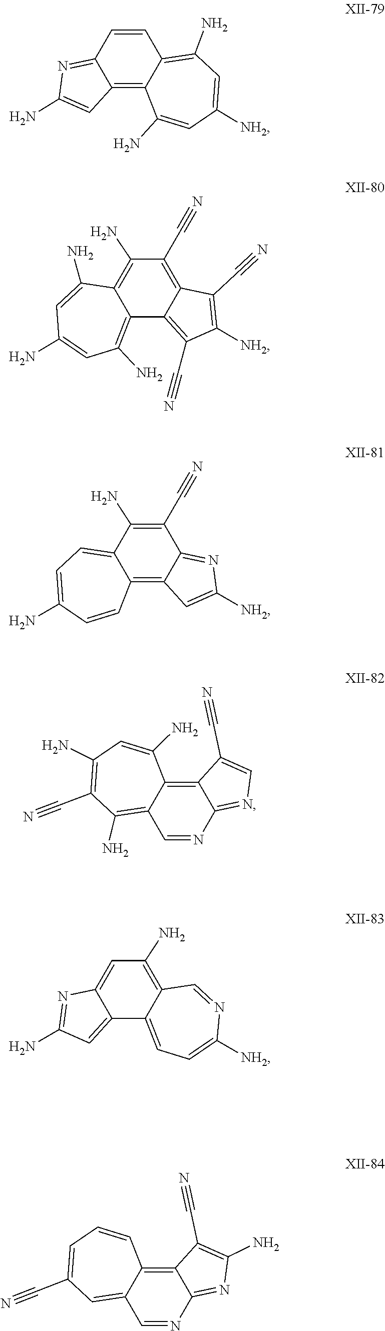 [SMILES: Nc1cc(N)c2ccc3nc(N)cc3c2c(N)c1 ; N#Cc1c(N)c(C#N)c2c1c(C#N)c(N)c1c(N)cc(N)cc(N)c12 ; N#Cc1c(N)c2ccc(N)ccc2c2cc(N)nc12 ; N#Cc1c(N)cc(N)c2c(cnc3ncc(C#N)c32)c1N ; Nc1ccc2c(cn1)c(N)cc1nc(N)cc12 ; N#Cc1cccc2c(cnc3nc(N)c(C#N)c32)c1]

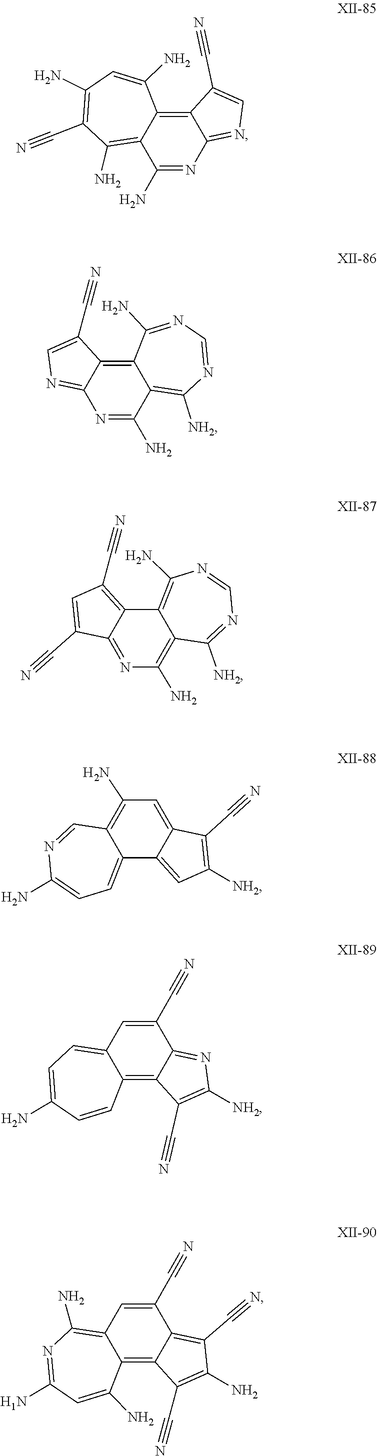

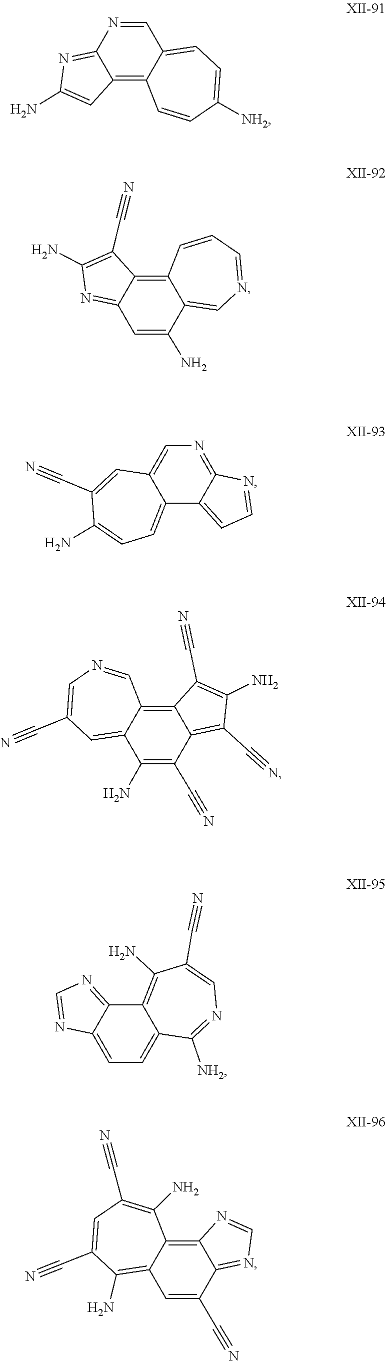 [SMILES: Nc1ccc2cnc3nc(N)cc3c2cc1 ; N#Cc1c(N)nc2cc(N)c3cncccc3c12 ; N#Cc1cc2cnc3nccc3c2ccc1N ; N#Cc1cncc2c(c1)c(N)c(C#N)c1c(C#N)c(N)c(C#N)c12 ; N#Cc1cnc(N)c2ccc3ncnc3c2c1N ; N#Cc1cc(C#N)c(N)c2c(cc(C#N)c3ncnc32)c1N]

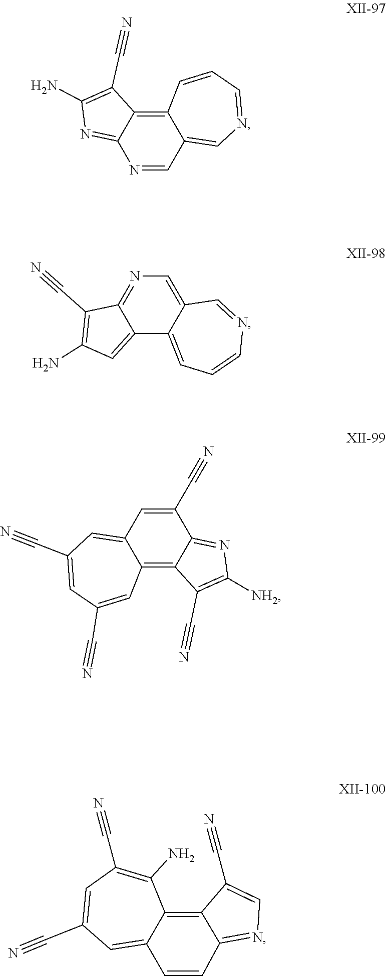 [SMILES: N#Cc1c(N)nc2ncc3cncccc3c12 ; N#Cc1c(N)cc2c1ncc1cncccc12 ; N#Cc1cc(C#N)cc2c(c1)cc(C#N)c1nc(N)c(C#N)c12 ; N#Cc1cc(C#N)c(N)c2c(ccc3ncc(C#N)c32)c1]

and a salt and/or tautomer thereof.

In another aspect, the present application includes a compound of Formula V or a salt and/or tautomer thereof

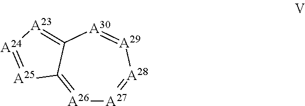

wherein:
A²³, A²⁴, A²⁵, A²⁶, A²⁷, A²⁸, A²⁹ and A³⁰ are each independently selected from C and N, and 0 to 4 of A²³, A²⁴, A²⁵, A²⁶, A²⁷, A²⁸, A²⁹ and A³⁰ are N; and each of A²³, A²⁴, A²⁵, A²⁸, A²⁷, A²⁸, A²⁹ and A³⁰ that is a C is independently unsubstituted or substituted with a substituent of Formula Va

-L-R¹ Va
wherein:
L is selected from a bond, O, S, NH, —CR^{a}=, —N═, C(O), C₂₋₆alkenylene, C₂₋₆alkenyleneO and C₂₋₆alkenyleneS;
when L is a bond, R¹ is selected from H, CN and halo;
when L is other than a bond, R¹ is selected from H, C₅₋₂₀heteroaryl, C₅₋₂₀cycloalkenyl, C₆₋₂₀aryl, C₄₋₂₀heterocyclyl and C₂-C₆alkenyl, the latter 5 groups being unsubstituted or substituted with one or more groups independently selected from halo, CN, OH, NH₂, SH, C(O)H, C₂₋₄alkenyl, C₂₋₄fluoroalkenyl, NHC₂₋₄alkenyl, NHC₂₋₄fluoroalkenyl, OC₂₋₄alkenyl, SC₂₋₄fluoroalkenyl, NH₂-substituted C₂₋₄alkenyl, C(O)H, OC(O)H, NHC(O)H, SC(O)H, and C(O)NH₂, and/or
with one or two groups independently selected from ═CHR^{a}, C₅-C₀heteroaryl, C₅₋₂₀cycloalkenyl, C₆₋₂₀aryl, C₄₋₂₀heterocyclyl, OC₅₋₂₀heteroaryl, OC₅₋₂₀cycloalkenyl, OC₆₋₂₀aryl, OC₄₋₂₀heterocyclyl, NHC₅₋₂₀heteroaryl, NHC₅₋₂₀cycloalkenyl, NHC₆₋₂₀aryl, NHC₄₋₂₀heterocyclyl, SC₅₋₂₀heteroaryl, SC₅₋₂₀cycloalkenyl, SC₆₋₂₀aryl, SC₄₋₂₀heterocyclyl, C(O)C₅₋₂₀heteroaryl, C(O)C₅₋₂₀cycloalkenyl, C(O)C₆₋₂₀aryl, C(O)C₄₋₂₀heterocyclyl, C₂₋₄alkenyleneC₅₋₂₀heteroaryl, C₂₋₄alkenyleneC₅₋₂₀cycloalkenyl, C₂₋₄alkenyleneC₆₋₂₀aryl and C₂₋₄alkenyleneC₄₋₂₀heterocyclyl, in which each cyclic group is unsubstituted or substituted with one or more groups independently selected from NH₂, CN, OH, SH, halo, C₂₋₄alkenyl and C(O)H;
R^{a} is selected from H, C₅₋₁₀heteroaryl, and C₆₋₁₀aryl, the latter two groups being unsubstituted or substituted with one or more group independently selected from NH₂, CN, OH, SH, halo, C₂₋₄alkenyl and C(O)H, and
the compound has a negative singlet-triplet gap and an oscillator strength greater than or equal to about 0.01.

In some embodiments, 1 to 4 of A²³, A²⁴, A²⁵, A²⁶, A²⁷, A²⁸, A²⁹ and A³⁰ are N. In some embodiments, 2 to 4 of A²³, A²⁴, A²⁵, A²⁶, A²⁷, A²⁸, A²⁹ and A³⁰ are N.

In some embodiments, A²⁴ is N. In some embodiments, one of A²³ and A²⁵ is N. In some embodiments, A²⁴ is N, and one of A²³ and A²⁵ is N. In some embodiments, A²⁴ is N, or one of A²³ and A²⁵ is N.

In some embodiments, at least one of A²⁹ and A³⁰ is N. In some embodiments, at least one of A²⁷ and A²⁹ is N. In some embodiments, at least one of A²⁹ and A³⁰ is N, and at least one of A²⁷ and A²⁹ is N.

In some embodiments, the compound of Formula V comprises 2 or 3 substituents of Formula Va. In some embodiments, the compound of Formula I comprises 2 substituents of Formula Va. In some embodiments, the two substituents of Formula Va are the same.

In some embodiments, L is selected from a bond, O, S, NH, —CR^{a}═, —N═, C₂₋₄alkenylene, C₂₋₄alkenyleneO and C₂₋₄alkenyleneS. In some embodiments, L is selected from a bond, O, S, NH, C₂alkenylene, C₂alkenyleneO and C₂alkenyleneS. In some embodiments, L is selected from O, S, and NH.

In some embodiments, L is other than a bond and R¹ is selected from C₅₋₁₀heteroaryl, C₅₋₁₀cycloalkenyl, C₆₋₁₀aryl, C₅₋₁₀heterocyclyl and C₂-C₄alkenyl, the latter 5 groups being unsubstituted or substituted with one to four groups independently selected from halo, CN, OH, NH₂, SH, C(O)H, C₂₋₄alkenyl, C₂₋₄fluoroalkenyl, NHC₂₋₄alkenyl, NHC₂₋₄fluoroalkenyl, OC₂₋₄alkenyl, SC₂₋₄fluoroalkenyl, NH₂-substituted C₂₋₄alkenyl, and C(O)H, and/or with one or two groups independently selected from =CHR^{a}, C₅-C₁₀heteroaryl, C₅₋₁₀cycloalkenyl, C₆₋₁₀aryl, C₄₋₁₀heterocyclyl, OC₅₋₁₀heteroaryl, OC₅₋₁₀cycloalkenyl, OC₆₋₂₀aryl, OC₄₋₁₀heterocyclyl, NHC₅₋₁₀heteroaryl, NHC₅₋₁₀cycloalkenyl, NHC₆₋₂₀aryl, NHC₄₋₁₀heterocyclyl, SC₅₋₁₀heteroaryl, SC₅₋₁₀cycloalkenyl, SC₆₋₁₀aryl, SC₄₋₁₀heterocyclyl, C(O)C₅₋₁₀heteroaryl, C(O)C₅₋₁₀cycloalkenyl, C(O)C₆₋₁₀aryl, C(O)C₄₋₁₀heterocycyl, C₂₋₄alkenyleneC₅₋₁₀heteroaryl, C₂₋₄alkenyleneC₅₋₁₀cycloalkenyl, C₂₋₄alkenyleneC₆₋₁₀aryl and C₂₋₄alkenyleneC₄₋₁₀heterocyclyl, in which each cyclic group is unsubstituted or substituted with one to five groups independently selected from NH₂, CN, OH, SH, halo, C₂₋₄alkenyl and C(O)H.

In some embodiments, L is other than a bond and R¹ is selected from C₅₋₁₀heteroaryl, C₅₋₁₀cycloalkenyl, C₆₋₁₀aryl, and C₅₋₁₀heterocyclyl, the latter 4 groups being unsubstituted or substituted with one to four groups independently selected from F, CN, OH, NH₂, SH, C(O)H, C₂alkenyl, C₂fluoroalkenyl, NHC₂alkenyl, NHC₂fluoroalkenyl, OC₂alkenyl, SC₂fluoroalkenyl, and C(O)H, and/or with one or two groups independently selected from =CHR^{a}, C₅-C₁₀heteroaryl, C₅₋₁₀cycloalkenyl, C₆₋₁₀aryl, C₄₋₁₀heterocyclyl, OC₅₋₁₀heteroaryl, OC₅₋₁₀cycloalkenyl, OC₆₋₂₀aryl, OC₄₋₁₀heterocyclyl, NHC₅₋₁₀heteroaryl, NHC₅₋₁₀cycloalkenyl, NHC₆₋₂₀aryl, NHC₄₋₁₀heterocyclyl, SC₅₋₁₀heteroaryl, SC₅₋₁₀cycloalkenyl, SC₆₋₁₀aryl, SC₄₋₁₀heterocyclyl, C(O)C₅₋₁₀heteroaryl, C(O)C₅₋₁₀cycloalkenyl, C(O)C₆₋₁₀aryl, C(O)C₄₋₁₀heterocycyl, C₂alkenyleneC₅₋₁₀heteroaryl, C₂alkenyleneC₅₋₁₀cycloalkenyl, C₂alkenyleneC₆₋₁₀aryl and C₂alkenyleneC₄₋₁₀heterocyclyl, in which each cyclic group is unsubstituted or substituted with one to four groups independently selected from NH₂, CN, OH, SH, halo, C₂₋₄alkenyl and C(O)H.

In some embodiments, each R¹ is independently selected from aryl and heteroaryl. In some embodiments, each R¹ is substituted with one to four substituents independently selected from OH, NH, SH, halo, and C₂-C₄alkene.

In some embodiments, each aryl is independently selected from 6-10-membered aryls. In some embodiments, each aryl is independently selected from naphthyl, phenyl, and azulenyl.

In some embodiments, each heteroaryl is independently selected from 5-16-membered heteroaryls comprising 1 to 4 heteroatoms independently selected from O, N, and S.

In embodiments, each heteroaryl is independently selected from imidazole, furan, pyrazole, pyrazine, thiophene, pyridine, pyrimidine, benzoimidazole, pyrroloisoquinoline, pyrrolopyridazine, pyrrolotriazine, cyclopentapyridine, cyclopentapyrrolopyridine, imidazoloisoquinoline, dihydrocinnolene, thiochromene, tetrazine, benzoisothiazole, isothiazolopyridine, benzofuroimidazole, dihydropyridazine, pyrroloquinoxaline, thienopyridine, pyrazolopyridine, benzopyrrole, cyclopentapyrridazine, pentalenopyridine, thiazine, furopyrazole, pyrazolopyridine, pyrroloquinoline, pyrrolopyrazine, oxazole, furopyrrole, cyclopentaindolizine, thienoisoxazole, thienopyridazine, thienoimidazole, thienofuran, acenaphthlene, thienopyridine, indolopyridine, benzothiazine, triazole, cyclopentapyrazole, cycloheptapyrrole, furoimidazole, indolizine, naphtharydine, pyrrolopyrazole, pyrazoloindole, cyclopentapyridine, furopyrrole, imidazoimidazole, cyclopentapyrimidine, pyrrolopyridine, benzoisoxazole, furopyridine, pyridoindole, cyclopenta-azepine, isoquinoline, quinoline, imidazopyridine, isothiazolopyridine, dihydroquinoxaline, imidazopyrazole, pyrrolo-oxazole, benzoisothiazole, pyrroloindole, pyridazine, pyrrolopyrimidine, pyrrolofuropyridine, imidazopyrrolopyridine, pyrrolopyridazine, benzothienopyridine, pyrroloisothiazole, quinazoline, isoxazolopyridine, pyrrolopyrrolopyridine, benzothienothiophene, imidazopyridine, thienopyrazole, and cycloheptaoxazepine.

In some embodiments, each heteroaryl is independently selected from

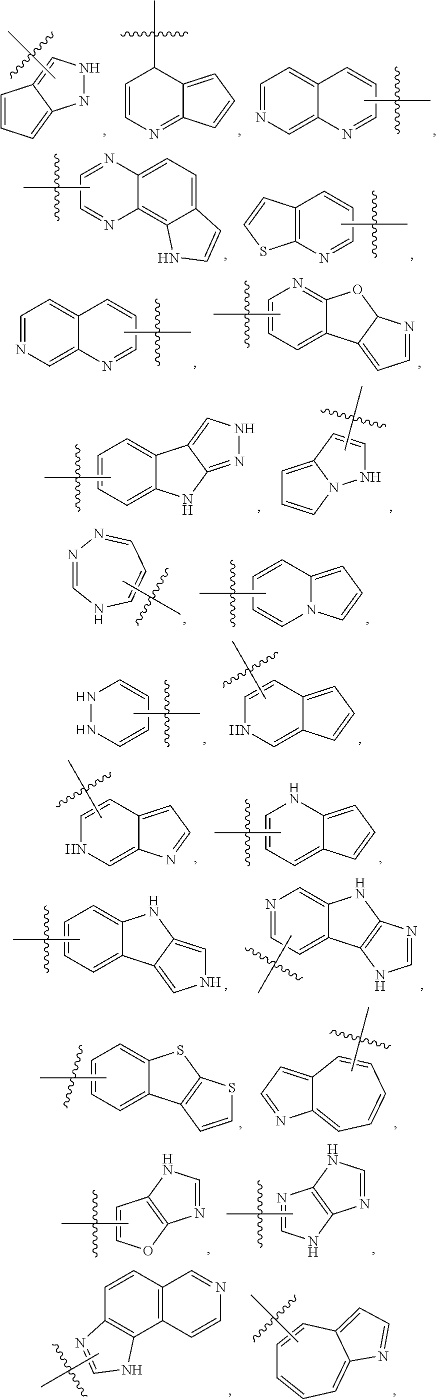

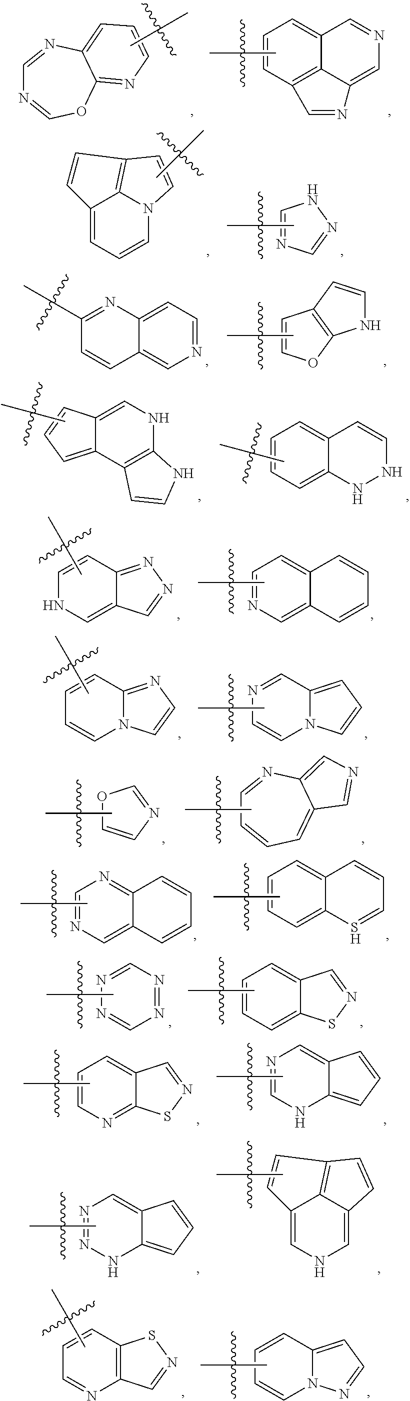

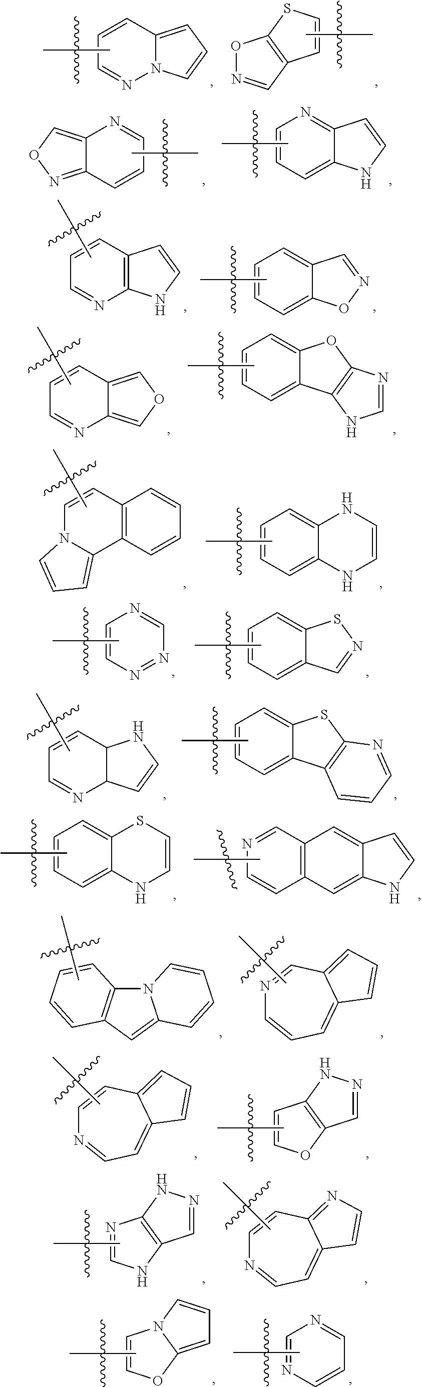

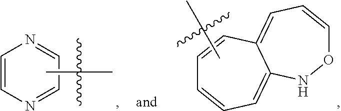

In some embodiments, each heterocyclyl is independently a 6-8-membered heterocyclyl comprising 1 to 3 heteroatoms selected from O, N, S, and combinations thereof. In some embodiments, the heterocyclyl is 1,2-diazepine.

In some embodiments, halo is selected from F, Cl, Br, and I. In some embodiments, halo is F or CI. In some embodiments halo is F.

In some embodiments, R^{a} is selected from H, C₅₋₁₀heteroaryl, and C₆₋₁₀aryl, the latter two groups being unsubstituted or substituted with one to four groups independently selected from NH₂, CN, OH, SH, halo, C₂₋₄alkenyl and C(O)H.

In some embodiments, the compound of Formula V is selected from the compounds in Table A and salt and/or tautomer thereof.

TABLE A
Exemplary compounds of Formula V

| | |
|---|---|
| 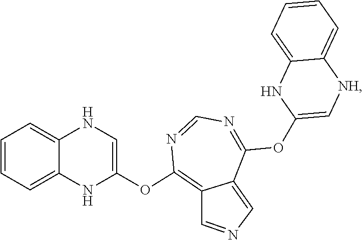 [SMILES: C1=C(Oc2ncnc(OC3=CNc4ccccc4N3)c3cncc2-3)Nc2ccccc2N1] | V-101 |
| 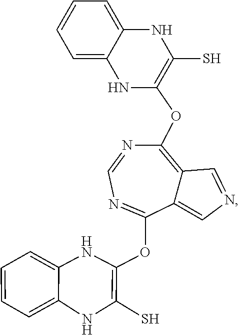 [SMILES: SC1=C(Oc2ncnc(OC3=C(S)Nc4ccccc4N3)c3cncc2-3)Nc2ccccc2N1] | V-102 |
| 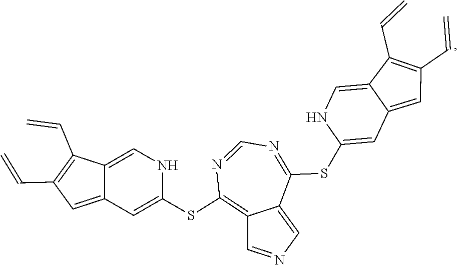 [SMILES: C=Cc1cc2cc(Sc3ncnc(Sc4cc5cc(C=C)c(C=C)c-5c[nH]4)c4cncc3-4)[nH]cc-2c1C=C] | V-103 |
| 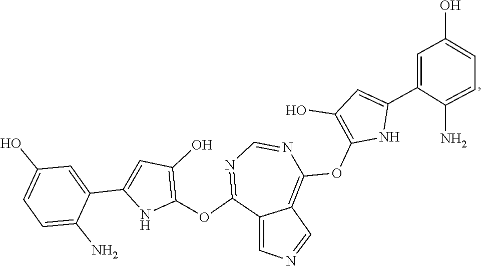 [SMILES: Nc1ccc(O)cc1-c1cc(O)c(Oc2ncnc(Oc3[nH]c(-c4cc(O)ccc4N)cc3O)c3cncc2-3)[nH]1] | V-104 |
| 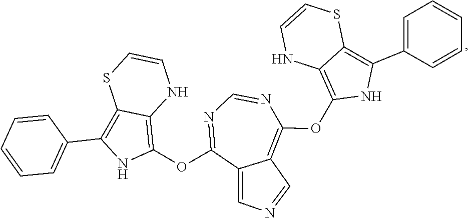 [SMILES: C1=CSc2c(-c3ccccc3)[nH]c(Oc3ncnc(Oc4[nH]c(-c5ccccc5)c5c4NC=CS5)c4cncc3-4)c2N1] | V-105 |
| 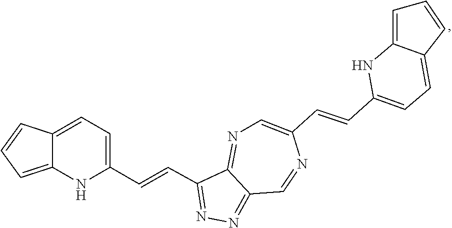 [SMILES: C(=C\c1ccc2cccc-2[nH]1)/c1cnc2c(/C=C/c3ccc4cccc-4[nH]3)nnc-2cn1] | V-106 |
| 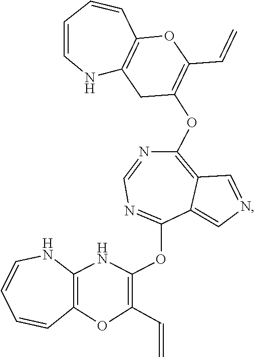 [SMILES: C=CC1=C(Oc2ncnc(Oc3[nH]c4[nH]ccccc=4oc3C=C)c3cncc2-3)CC2=C(C=CC=CN2)O1] | V-107 |
| 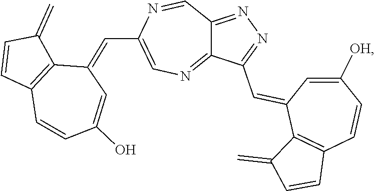 [SMILES: C=c1ccc2ccc(O)c/c(=C\c3cnc4c(/C=c5\cc(O)ccc6ccc(=C)c5=6)nnc-4cn3)c1=2] | V-108 |
| 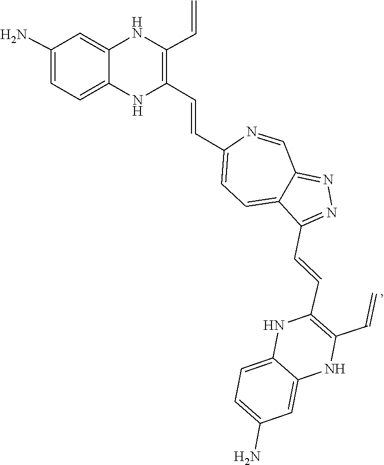 [SMILES: C=CC1=C(/C=C/c2ccc3c(/C=C/C4=C(C=C)Nc5cc(N)ccc5N4)nnc-3cn2)Nc2ccc(N)cc2N1] | V-109 |
| 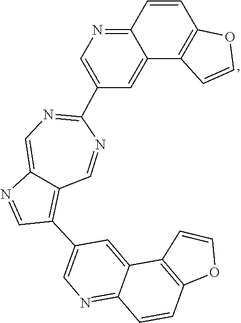 [SMILES: c1cc2c(ccc3ncc(-c4ncc5ncc(-c6cnc7ccc8occc8c7c6)c-5cn4)cc32)o1] | V-110 |
| 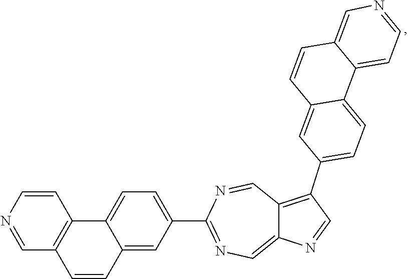 [SMILES: c1cc2c(ccc3cc(-c4ncc5ncc(-c6ccc7c(ccc8cnccc87)c6)c-5cn4)ccc32)cn1] | V-111 |
| 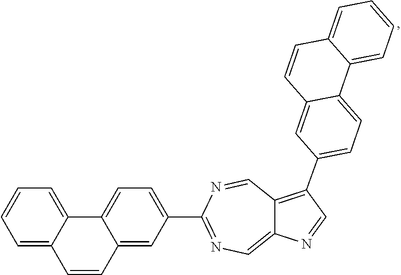 [SMILES: c1ccc2c(c1)ccc1cc(-c3ncc4ncc(-c5ccc6c(ccc7ccccc76)c5)c-4cn3)ccc12] | V-112 |
| 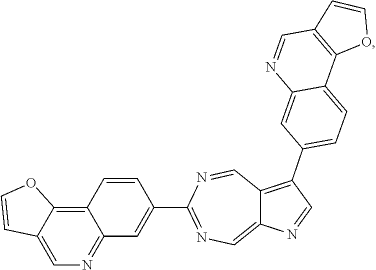 [SMILES: c1cc2cnc3cc(-c4ncc5ncc(-c6ccc7c(c6)ncc6ccoc67)c-5cn4)ccc3c2o1] | V-113 |
| 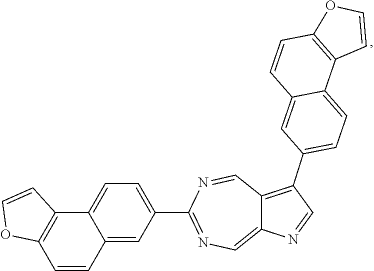 [SMILES: c1cc2c(ccc3cc(-c4ncc5ncc(-c6ccc7c(ccc8occc87)c6)c-5cn4)ccc32)o1] | V-114 |
| 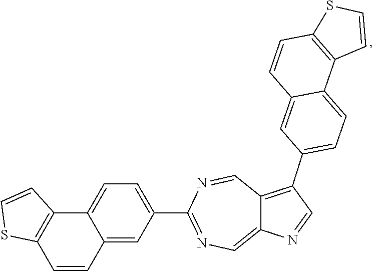 [SMILES: c1cc2c(ccc3cc(-c4ncc5ncc(-c6ccc7c(ccc8sccc87)c6)c-5cn4)ccc32)s1] | V-115 |
| 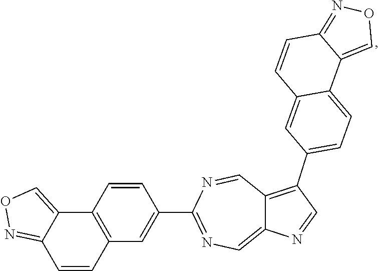 [SMILES: c1cc2c(ccc3nocc32)cc1-c1ncc2ncc(-c3ccc4c(ccc5nocc54)c3)c-2cn1] | V-116 |
| 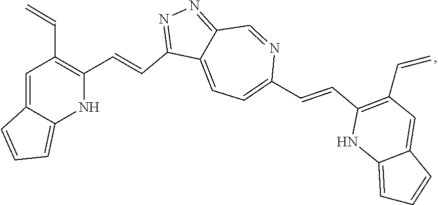 [SMILES: C=Cc1cc2cccc-2[nH]c1/C=C/c1ccc2c(/C=C/c3[nH]c4cccc-4cc3C=C)nnc-2cn1] | V-117 |
| 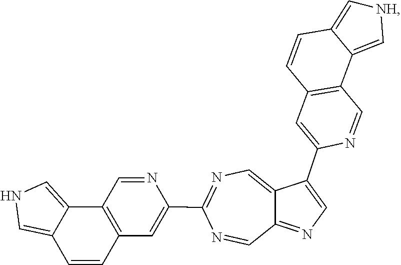 [SMILES: c1nc(-c2cc3ccc4c[nH]cc4c3cn2)ncc2c(-c3cc4ccc5c[nH]cc5c4cn3)cnc1-2] | V-118 |
| 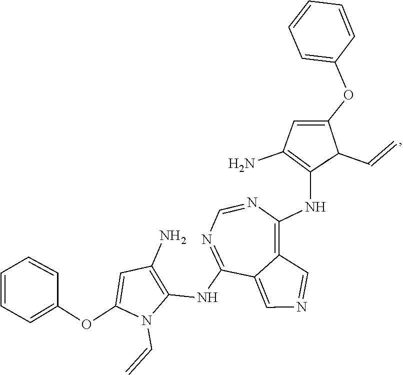 | V-119 |
| 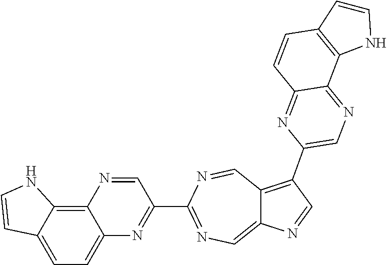 [SMILES: c1cc2ccc3nc(-c4ncc5ncc(-c6cnc7c(ccc8cc[nH]c87)n6)c-5cn4)cnc3c2[nH]1] | V-120 |
| 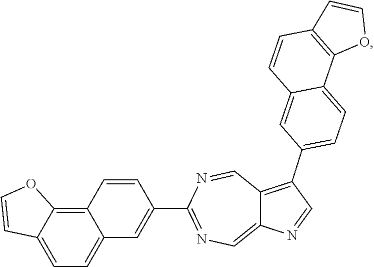 [SMILES: c1cc2ccc3cc(-c4ncc5ncc(-c6ccc7c(ccc8ccoc87)c6)c-5cn4)ccc3c2o1] | V-121 |
| 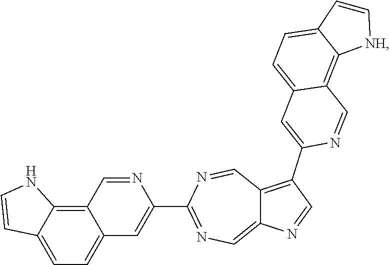 [SMILES: c1cc2ccc3cc(-c4ncc5ncc(-c6cc7ccc8cc[nH]c8c7cn6)c-5cn4)ncc3c2[nH]1] | V-122 |
| 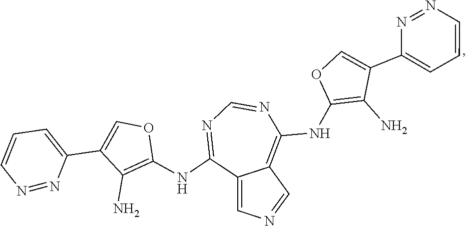 [SMILES: Nc1c(-c2cccnn2)coc1Nc1ncnc(Nc2occ(-c3cccnn3)c2N)c2cncc1-2] | V-123 |
| 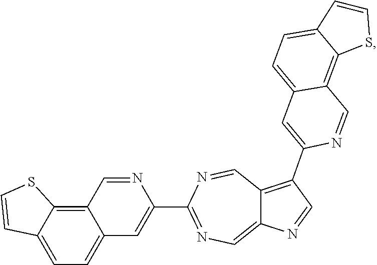 [SMILES: c1cc2ccc3cc(-c4ncc5ncc(-c6cc7ccc8ccsc8c7cn6)c-5cn4)ncc3c2s1] | V-124 |
| 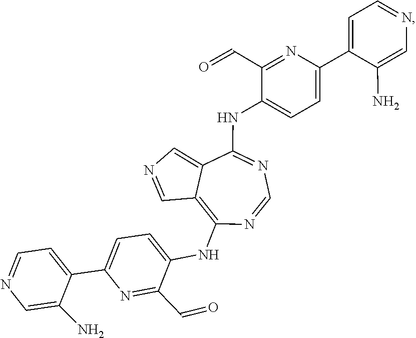 [SMILES: Nc1cnccc1-c1ccc(Nc2ncnc(Nc3ccc(-c4ccncc4N)nc3C=O)c3cncc2-3)c(C=O)n1] | V-125 |
| 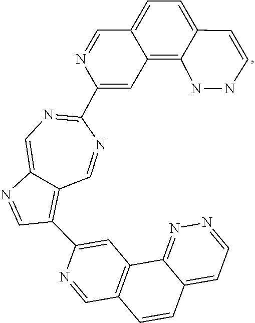 | V-126 |
| 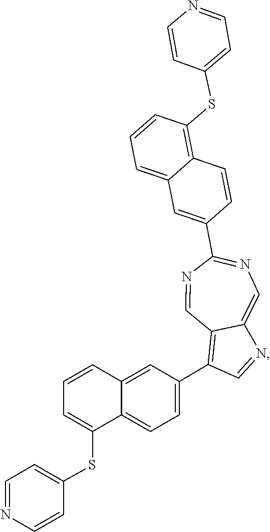 [SMILES: c1cc(Sc2ccncc2)c2ccc(-c3ncc4ncc(-c5ccc6c(Sc7ccncc7)cccc6c5)c-4cn3)cc2c1] | V-127 |
| 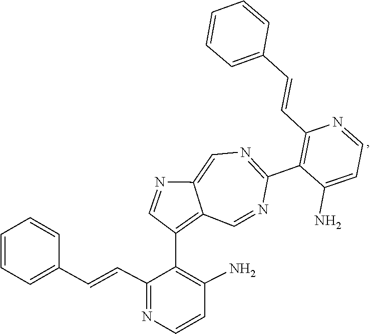 [SMILES: Nc1ccnc(/C=C/c2ccccc2)c1-c1ncc2ncc(-c3c(N)ccnc3/C=C/c3ccccc3)c-2cn1] | V-128 |
| 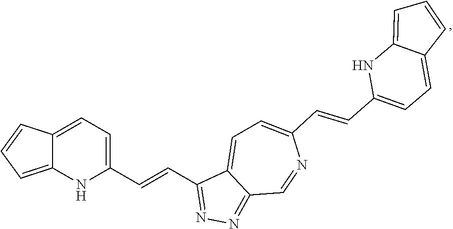 [SMILES: C(=C\c1ccc2cccc-2[nH]1)/c1ccc2c(/C=C/c3ccc4cccc-4[nH]3)nnc-2cn1] | V-129 |
| 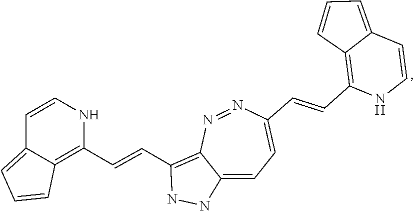 | V-130 |
| 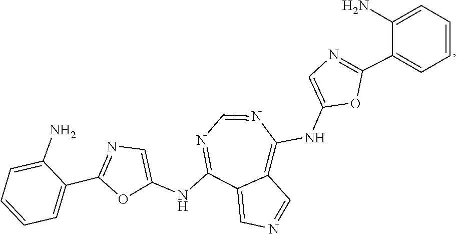 [SMILES: Nc1ccccc1-c1ncc(Nc2ncnc(Nc3cnc(-c4ccccc4N)o3)c3cncc2-3)o1] | V-131 |
| 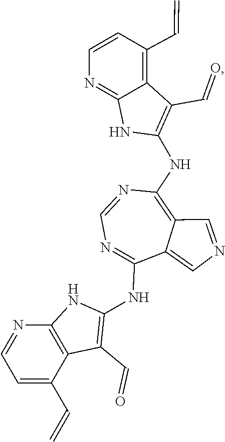 [SMILES: C=Cc1ccnc2[nH]c(Nc3ncnc(Nc4[nH]c5nccc(C=C)c5c4C=O)c4cncc3-4)c(C=O)c12] | V-132 |
| 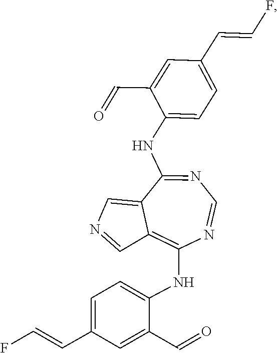 [SMILES: O=Cc1cc(/C=C/F)ccc1Nc1ncnc(Nc2ccc(/C=C/F)cc2C=O)c2cncc1-2] | V-133 |
| 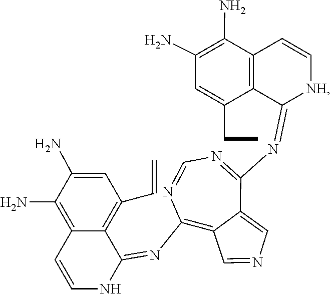 | V-134 |
| 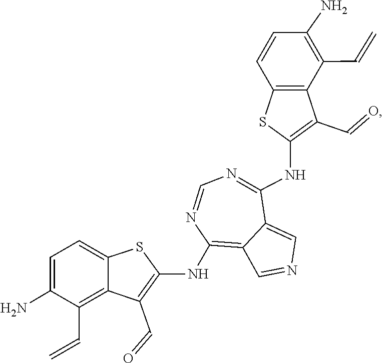 [SMILES: C=Cc1c(N)ccc2sc(Nc3ncnc(Nc4sc5ccc(N)c(C=C)c5c4C=O)c4cncc3-4)c(C=O)c12] | V-135 |
| 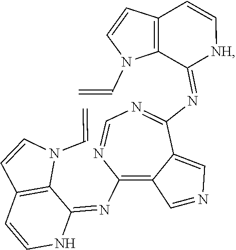 [SMILES: C=Cn1ccc2cc[nH]/c(=N/c3ncnc(/N=c4/[nH]ccc5ccn(C=C)c45)c4cncc3-4)c21] | V-136 |
| 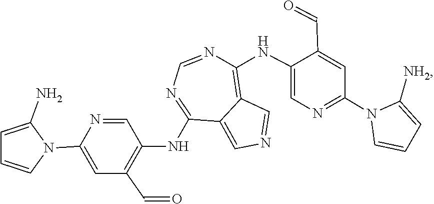 [SMILES: Nc1cccn1-c1cc(C=O)c(Nc2ncnc(Nc3cnc(-n4cccc4N)cc3C=O)c3cncc2-3)cn1] | V-137 |
| 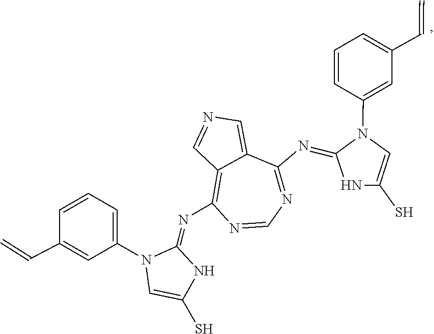 [SMILES: C=Cc1cccc(-n2cc(S)[nH]/c2=N\c2ncnc(/N=c3\[nH]c(S)cn3-c3cccc(C=C)c3)c3cncc2-3)c1] | V-138 |
| 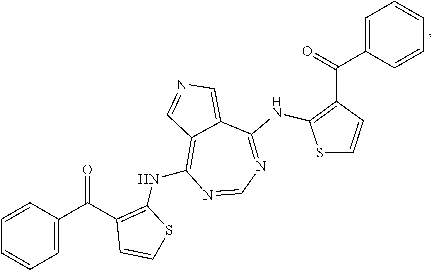 [SMILES: O=C(c1ccccc1)c1ccsc1Nc1ncnc(Nc2sccc2C(=O)c2ccccc2)c2cncc1-2] | V-139 |
| 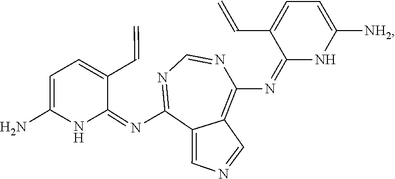 [SMILES: C=Cc1ccc(N)[nH]/c1=N/c1ncnc(/N=c2/[nH]c(N)ccc2C=C)c2cncc1-2] | V-140 |
| 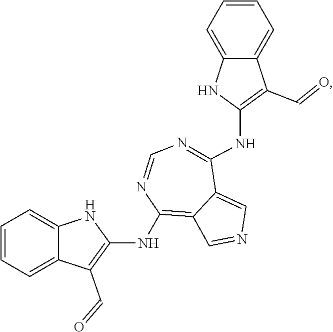 [SMILES: O=Cc1c(Nc2ncnc(Nc3[nH]c4ccccc4c3C=O)c3cncc2-3)[nH]c2ccccc12] | V-141 |
| 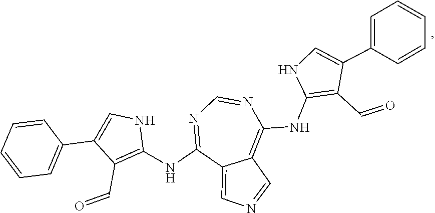 [SMILES: O=Cc1c(-c2ccccc2)c[nH]c1Nc1ncnc(Nc2[nH]cc(-c3ccccc3)c2C=O)c2cncc1-2] | V-142 |
| 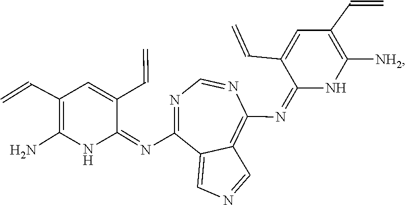 [SMILES: C=Cc1cc(C=C)/c(=N\c2ncnc(/N=c3/[nH]c(N)c(C=C)cc3C=C)c3cncc2-3)[nH]c1N] | V-143 |
| 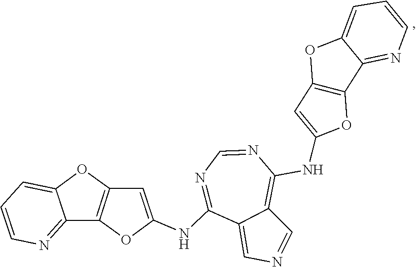 [SMILES: c1cnc2c(c1)oc1cc(Nc3ncnc(Nc4cc5oc6cccnc6c5o4)c4cncc3-4)oc12] | V-144 |
| 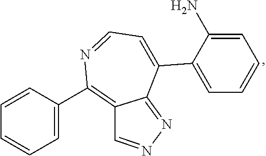 [SMILES: Nc1ccccc1-c1ccnc(-c2ccccc2)c2cnnc1-2] | V-145 |
| 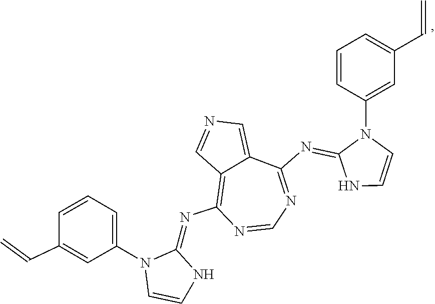 [SMILES: C=Cc1cccc(-n2cc[nH]/c2=N\c2ncnc(/N=c3\[nH]ccn3-c3cccc(C=C)c3)c3cncc2-3)c1] | V-146 |
| 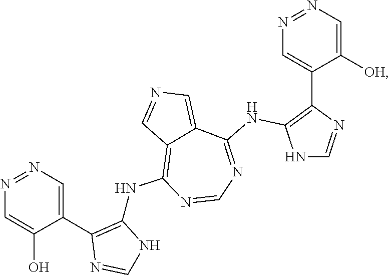 [SMILES: Oc1cnncc1-c1nc[nH]c1Nc1ncnc(Nc2[nH]cnc2-c2cnncc2O)c2cncc1-2] | V-147 |
| 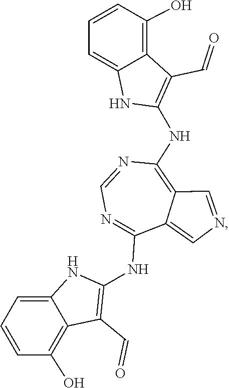 [SMILES: O=Cc1c(Nc2ncnc(Nc3[nH]c4cccc(O)c4c3C=O)c3cncc2-3)[nH]c2cccc(O)c12] | V-148 |
| 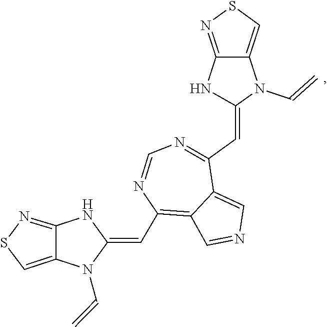 | V-149 |
| 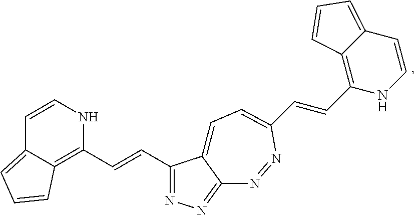 [SMILES: C(=C\c1[nH]ccc2cccc1-2)/c1ccc2c(/C=C/c3[nH]ccc4cccc3-4)nnc-2nn1] | V-150 |
| 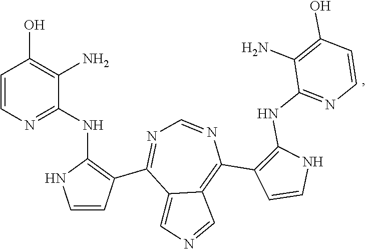 [SMILES: Nc1c(O)ccnc1Nc1[nH]ccc1-c1ncnc(-c2cc[nH]c2Nc2nccc(O)c2N)c2cncc1-2] | V-151 |
| 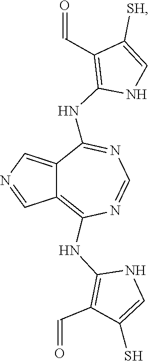 [SMILES: O=Cc1c(S)c[nH]c1Nc1ncnc(Nc2[nH]cc(S)c2C=O)c2cncc1-2] | V-152 |
| 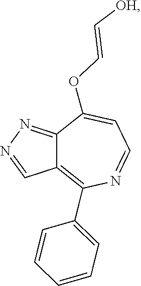 [SMILES: O/C=C/Oc1ccnc(-c2ccccc2)c2cnnc1-2] | V-153 |
| 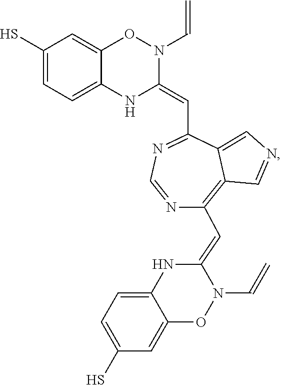 [SMILES: C=CN1Oc2cc(S)ccc2N/C1=C\c1ncnc(/C=C2\Nc3ccc(S)cc3ON2C=C)c2cncc1-2] | V-154 |
| 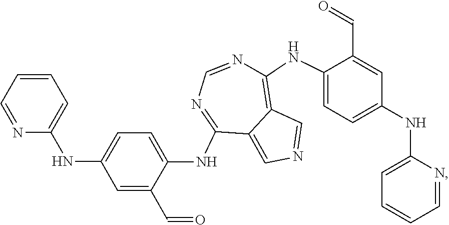 [SMILES: O=Cc1cc(Nc2ccccn2)ccc1Nc1ncnc(Nc2ccc(Nc3ccccn3)cc2C=O)c2cncc1-2] | V-155 |
| 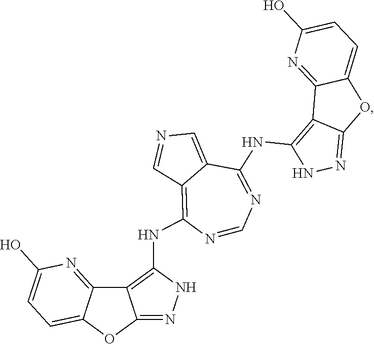 [SMILES: Oc1ccc2oc3n[nH]c(Nc4ncnc(Nc5[nH]nc6oc7ccc(O)nc7c56)c5cncc4-5)c3c2n1] | V-156 |
| 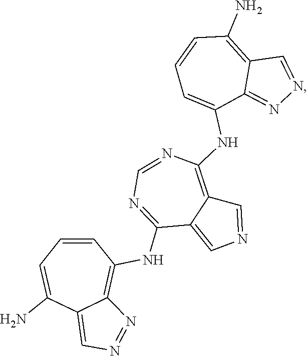 [SMILES: Nc1cccc(Nc2ncnc(Nc3cccc(N)c4cnnc3-4)c3cncc2-3)c2nncc1-2] | V-157 |
| 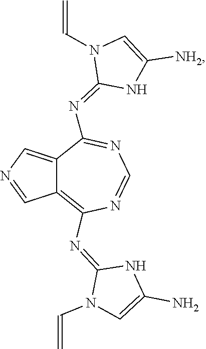 [SMILES: C=Cn1cc(N)[nH]/c1=N\c1ncnc(/N=c2\[nH]c(N)cn2C=C)c2cncc1-2] | V-158 |
| 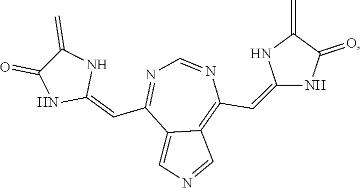 [SMILES: C=c1[nH]/c(=C\c2ncnc(/C=c3\[nH]c(=C)c(=O)[nH]3)c3cncc2-3)[nH]c1=O] | V-159 |
| 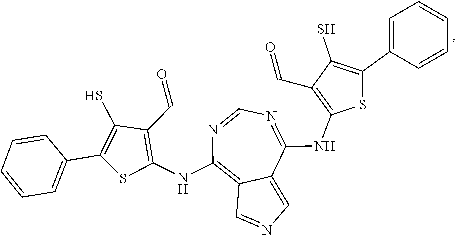 [SMILES: O=Cc1c(Nc2ncnc(Nc3sc(-c4ccccc4)c(S)c3C=O)c3cncc2-3)sc(-c2ccccc2)c1S] | V-160 |
| 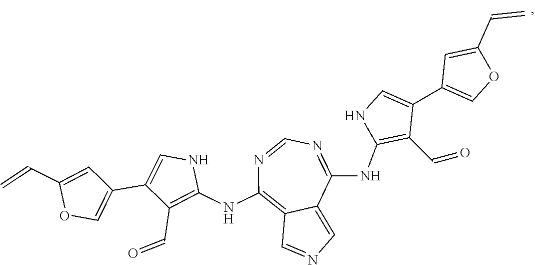 [SMILES: C=Cc1cc(-c2c[nH]c(Nc3ncnc(Nc4[nH]cc(-c5coc(C=C)c5)c4C=O)c4cncc3-4)c2C=O)co1] | V-161 |
| 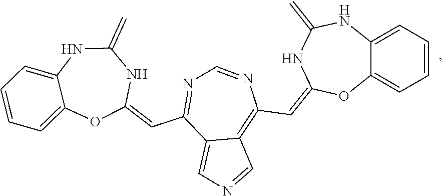 [SMILES: C=c1[nH]/c(=C\c2ncnc(/C=c3\[nH]c(=C)[nH]c4ccccc4o3)c3cncc2-3)oc2ccccc2[nH]1] | V-162 |
| 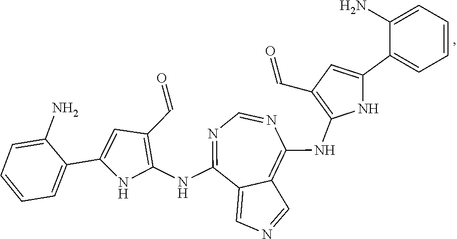 [SMILES: Nc1ccccc1-c1cc(C=O)c(Nc2ncnc(Nc3[nH]c(-c4ccccc4N)cc3C=O)c3cncc2-3)[nH]1] | V-163 |
| 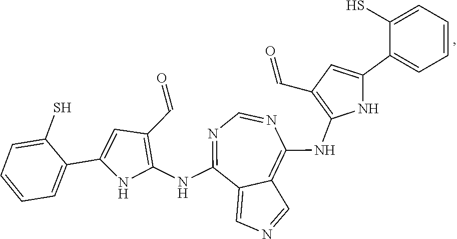 [SMILES: O=Cc1cc(-c2ccccc2S)[nH]c1Nc1ncnc(Nc2[nH]c(-c3ccccc3S)cc2C=O)c2cncc1-2] | V-164 |
| 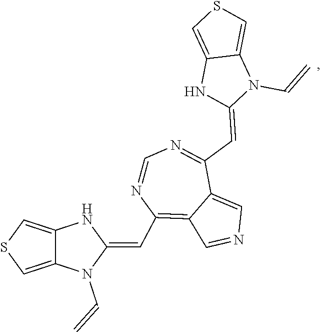 [SMILES: C=CN1/C(=C/c2ncnc(/C=C3\Nc4cscc4N3C=C)c3cncc2-3)Nc2cscc21] | V-165 |
| 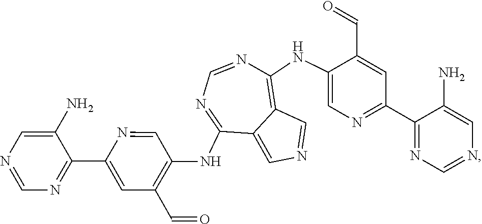 [SMILES: Nc1cncnc1-c1cc(C=O)c(Nc2ncnc(Nc3cnc(-c4ncncc4N)cc3C=O)c3cncc2-3)cn1] | V-166 |
| 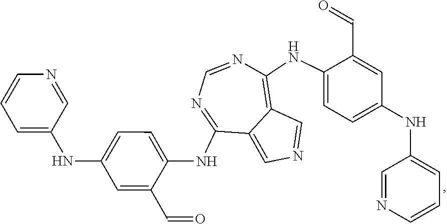 [SMILES: O=Cc1cc(Nc2cccnc2)ccc1Nc1ncnc(Nc2ccc(Nc3cccnc3)cc2C=O)c2cncc1-2] | V-167 |
| 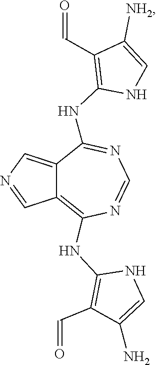 [SMILES: Nc1c[nH]c(Nc2ncnc(Nc3[nH]cc(N)c3C=O)c3cncc2-3)c1C=O] | V-168 |
| 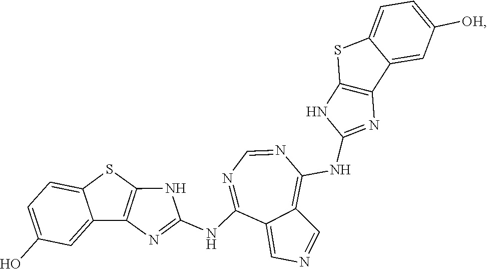 [SMILES: Oc1ccc2sc3[nH]c(Nc4ncnc(Nc5nc6c([nH]5)sc5ccc(O)cc56)c5cncc4-5)nc3c2c1] | V-169 |
| 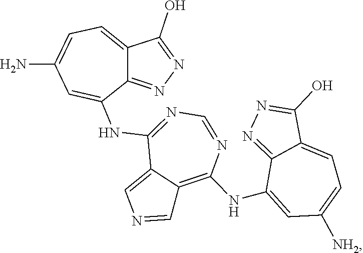 [SMILES: Nc1ccc2c(O)nnc-2c(Nc2ncnc(Nc3cc(N)ccc4c(O)nnc3-4)c3cncc2-3)c1] | V-170 |
| 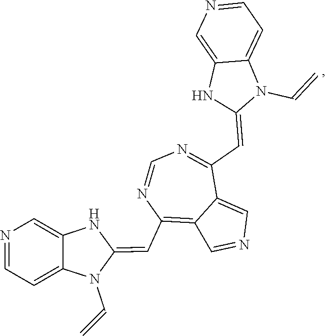 [SMILES: C=CN1/C(=C/c2ncnc(/C=C3\Nc4cnccc4N3C=C)c3cncc2-3)Nc2cnccc21] | V-171 |
| 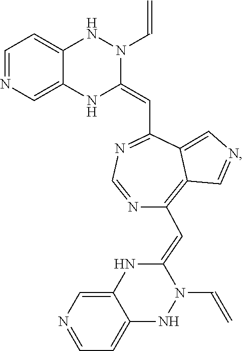 [SMILES: C=CN1Nc2ccncc2N/C1=C\c1ncnc(/C=C2\Nc3cnccc3NN2C=C)c2cncc1-2] | V-172 |
| 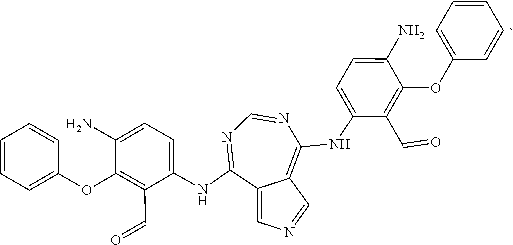 [SMILES: Nc1ccc(Nc2ncnc(Nc3ccc(N)c(Oc4ccccc4)c3C=O)c3cncc2-3)c(C=O)c1Oc1ccccc1] | V-173 |
| 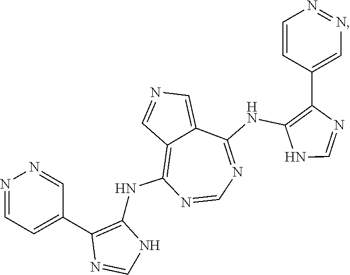 [SMILES: c1cc(-c2nc[nH]c2Nc2ncnc(Nc3[nH]cnc3-c3ccnnc3)c3cncc2-3)cnn1] | V-174 |
| 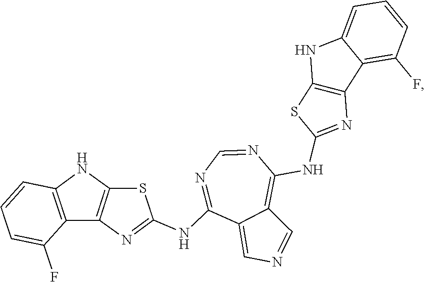 [SMILES: Fc1cccc2[nH]c3sc(Nc4ncnc(Nc5nc6c([nH]c7cccc(F)c76)s5)c5cncc4-5)nc3c12] | V-175 |
| 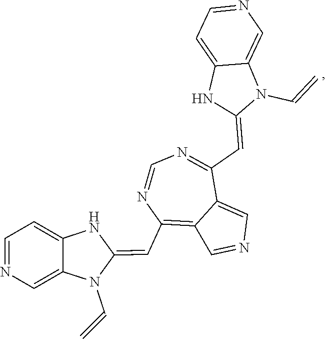 [SMILES: C=CN1/C(=C/c2ncnc(/C=C3\Nc4ccncc4N3C=C)c3cncc2-3)Nc2ccncc21] | V-176 |
| 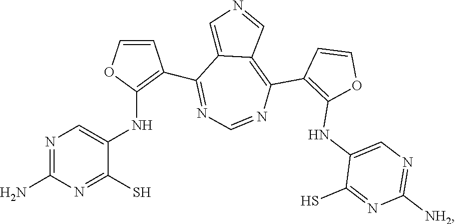 [SMILES: Nc1ncc(Nc2occc2-c2ncnc(-c3ccoc3Nc3cnc(N)nc3S)c3cncc2-3)c(S)n1] | V-177 |
| 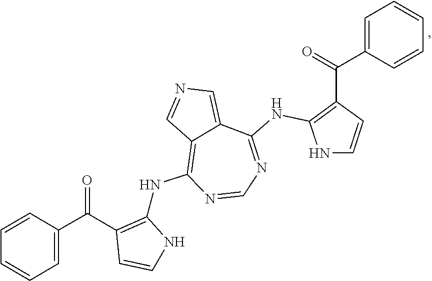 [SMILES: O=C(c1ccccc1)c1cc[nH]c1Nc1ncnc(Nc2[nH]ccc2C(=O)c2ccccc2)c2cncc1-2] | V-178 |
| 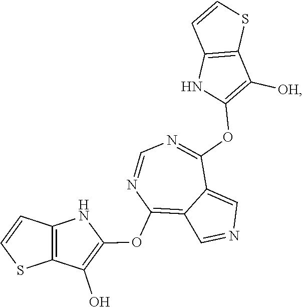 [SMILES: Oc1c(Oc2ncnc(Oc3[nH]c4ccsc4c3O)c3cncc2-3)[nH]c2ccsc12] | V-179 |
| 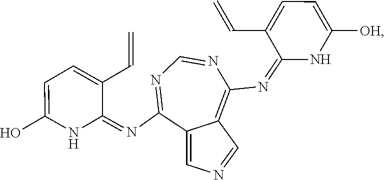 [SMILES: C=Cc1ccc(O)[nH]/c1=N/c1ncnc(/N=c2/[nH]c(O)ccc2C=C)c2cncc1-2] | V-180 |
| 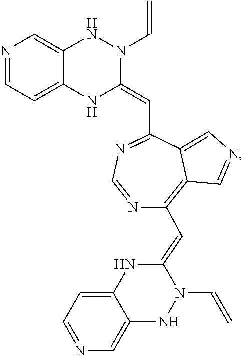 [SMILES: C=CN1Nc2cnccc2N/C1=C\c1ncnc(/C=C2\Nc3ccncc3NN2C=C)c2cncc1-2] | V-181 |
| 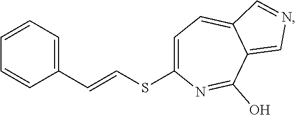 [SMILES: Oc1nc(S/C=C/c2ccccc2)ccc2cncc1-2] | V-182 |
| 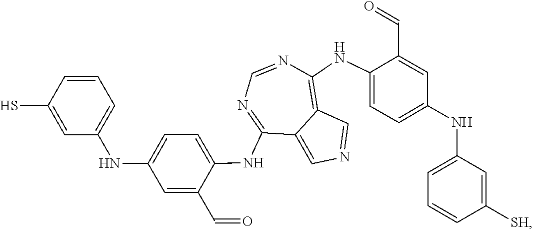 [SMILES: O=Cc1cc(Nc2cccc(S)c2)ccc1Nc1ncnc(Nc2ccc(Nc3cccc(S)c3)cc2C=O)c2cncc1-2] | V-183 |
| 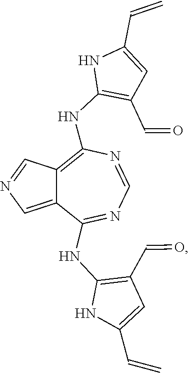 [SMILES: C=Cc1cc(C=O)c(Nc2ncnc(Nc3[nH]c(C=C)cc3C=O)c3cncc2-3)[nH]1] | V-184 |
| 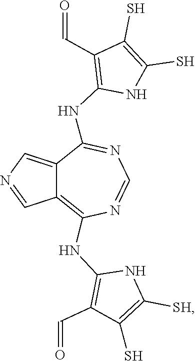 [SMILES: O=Cc1c(Nc2ncnc(Nc3[nH]c(S)c(S)c3C=O)c3cncc2-3)[nH]c(S)c1S] | V-185 |
| 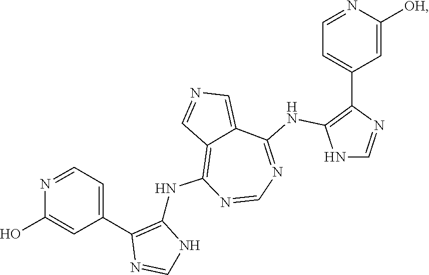 [SMILES: Oc1cc(-c2nc[nH]c2Nc2ncnc(Nc3[nH]cnc3-c3ccnc(O)c3)c3cncc2-3)ccn1] | V-186 |
| 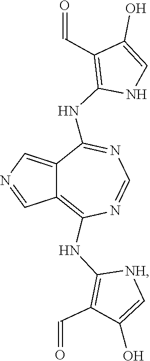 [SMILES: O=Cc1c(O)c[nH]c1Nc1ncnc(Nc2[nH]cc(O)c2C=O)c2cncc1-2] | V-187 |
| 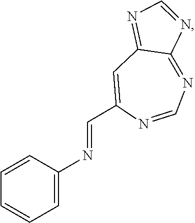 [SMILES: C(=N\c1ccccc1)/c1cc2ncnc-2ncn1] | V-188 |
| 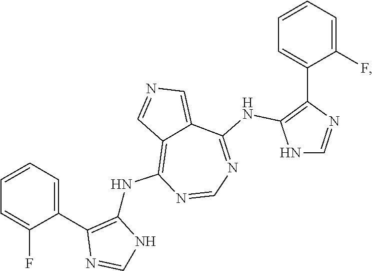 [SMILES: Fc1ccccc1-c1nc[nH]c1Nc1ncnc(Nc2[nH]cnc2-c2ccccc2F)c2cncc1-2] | V-189 |
| 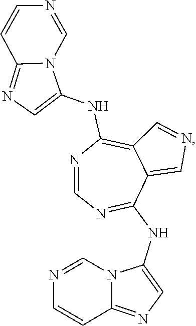 [SMILES: c1cc2ncc(Nc3ncnc(Nc4cnc5ccncn45)c4cncc3-4)n2cn1] | V-190 |
| 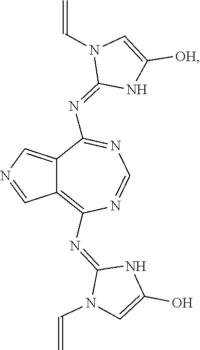 [SMILES: C=Cn1cc(O)[nH]/c1=N\c1ncnc(/N=c2\[nH]c(O)cn2C=C)c2cncc1-2] | V-191 |
| 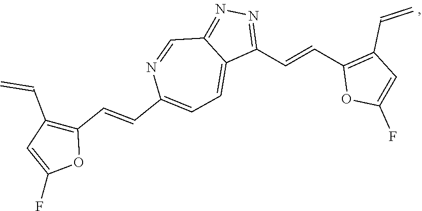 [SMILES: C=Cc1cc(F)oc1/C=C/c1ccc2c(/C=C/c3oc(F)cc3C=C)nnc-2cn1] | V-192 |
| 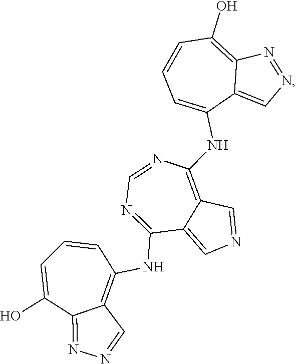 [SMILES: Oc1cccc(Nc2ncnc(Nc3cccc(O)c4nncc3-4)c3cncc2-3)c2cnnc1-2] | V-193 |
| 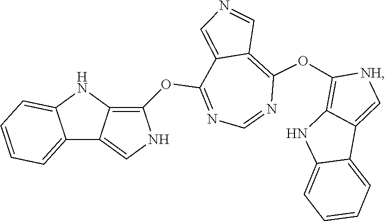 [SMILES: c1ccc2c(c1)[nH]c1c(Oc3ncnc(Oc4[nH]cc5c4[nH]c4ccccc45)c4cncc3-4)[nH]cc12] | V-194 |
| 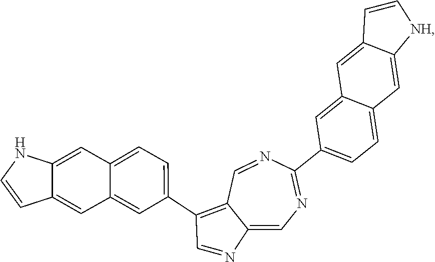 [SMILES: c1cc2cc3cc(-c4ncc5ncc(-c6ccc7cc8[nH]ccc8cc7c6)c-5cn4)ccc3cc2[nH]1] | V-195 |
| 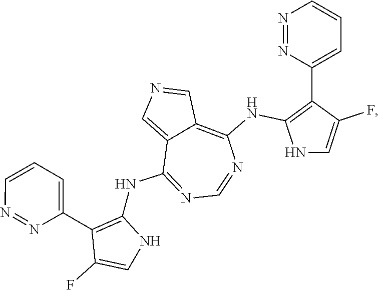 [SMILES: Fc1c[nH]c(Nc2ncnc(Nc3[nH]cc(F)c3-c3cccnn3)c3cncc2-3)c1-c1cccnn1] | V-196 |
| 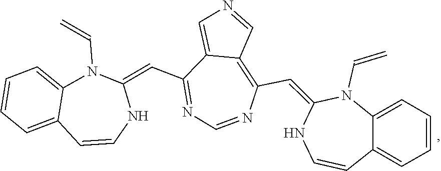 [SMILES: C=CN1/C(=C/c2ncnc(/C=C3\NC=Cc4ccccc4N3C=C)c3cncc2-3)NC=Cc2ccccc21] | V-197 |
| 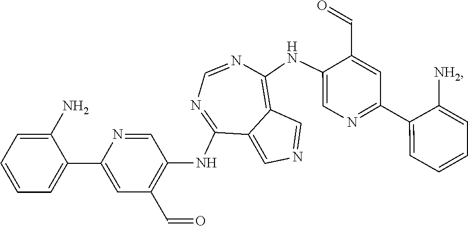 [SMILES: Nc1ccccc1-c1cc(C=O)c(Nc2ncnc(Nc3cnc(-c4ccccc4N)cc3C=O)c3cncc2-3)cn1] | V-198 |
| 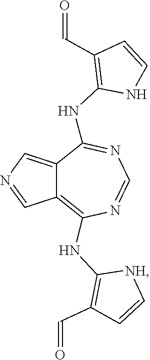 [SMILES: O=Cc1cc[nH]c1Nc1ncnc(Nc2[nH]ccc2C=O)c2cncc1-2] | V-199 |
| 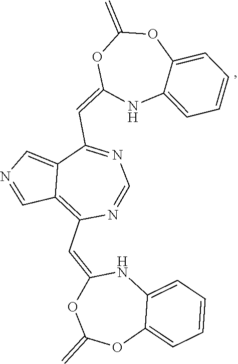 [SMILES: C=c1o/c(=C/c2ncnc(/C=c3\[nH]c4ccccc4oc(=C)o3)c3cncc2-3)[nH]c2ccccc2o1] | V-200 |
| 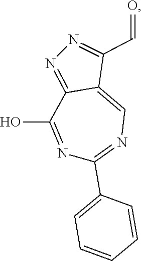 [SMILES: O=Cc1nnc2c(O)nc(-c3ccccc3)ncc1-2] | V-201 |
| 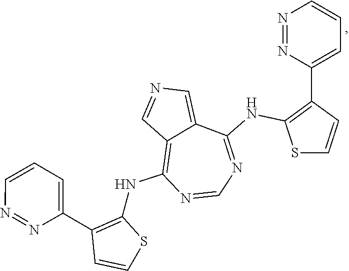 [SMILES: c1cnnc(-c2ccsc2Nc2ncnc(Nc3sccc3-c3cccnn3)c3cncc2-3)c1] | V-202 |
| 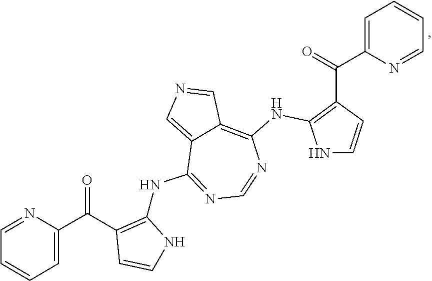 [SMILES: O=C(c1ccccn1)c1cc[nH]c1Nc1ncnc(Nc2[nH]ccc2C(=O)c2ccccn2)c2cncc1-2] | V-203 |
| 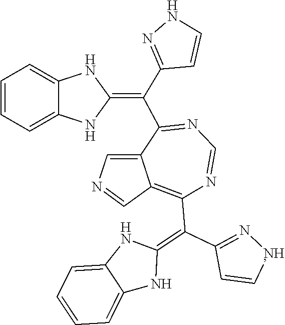 [SMILES: c1ccc2c(c1)NC(=C(c1cc[nH]n1)c1ncnc(C(=C3Nc4ccccc4N3)c3cc[nH]n3)c3cncc1-3)N2] | V-204 |
| 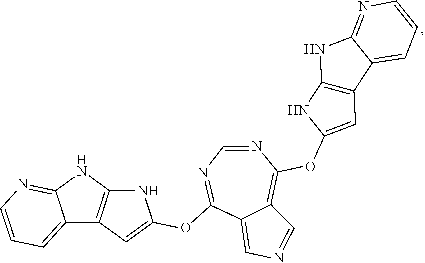 [SMILES: c1cnc2[nH]c3[nH]c(Oc4ncnc(Oc5cc6c([nH]5)[nH]c5ncccc56)c5cncc4-5)cc3c2c1] | V-205 |
| 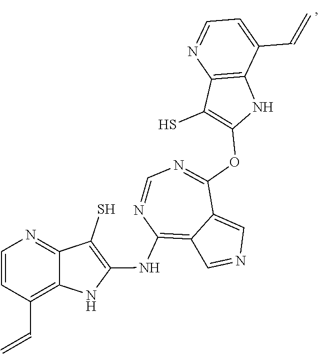 [SMILES: C=Cc1ccnc2c(S)c(Nc3ncnc(Oc4[nH]c5c(C=C)ccnc5c4S)c4cncc3-4)[nH]c12] | V-206 |
| 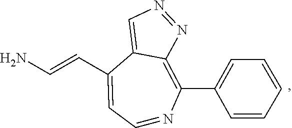 [SMILES: N/C=C/c1ccnc(-c2ccccc2)c2nncc1-2] | V-207 |
| 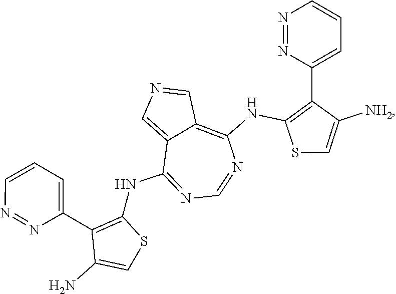 [SMILES: Nc1csc(Nc2ncnc(Nc3scc(N)c3-c3cccnn3)c3cncc2-3)c1-c1cccnn1] | V-208 |
| 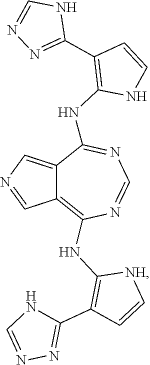 [SMILES: c1nc(Nc2[nH]ccc2-c2nnc[nH]2)c2cncc-2c(Nc2[nH]ccc2-c2nnc[nH]2)n1] | V-209 |
| 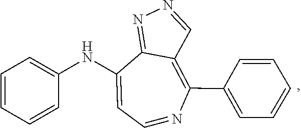 [SMILES: c1ccc(Nc2ccnc(-c3ccccc3)c3cnnc2-3)cc1] | V-210 |
| 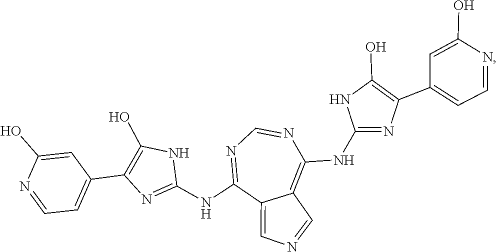 [SMILES: Oc1cc(-c2nc(Nc3ncnc(Nc4nc(-c5ccnc(O)c5)c(O)[nH]4)c4cncc3-4)[nH]c2O)ccn1] | V-211 |
| 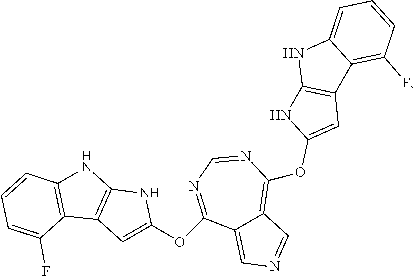 [SMILES: Fc1cccc2[nH]c3[nH]c(Oc4ncnc(Oc5cc6c([nH]5)[nH]c5cccc(F)c56)c5cncc4-5)cc3c12] | V-212 |
| 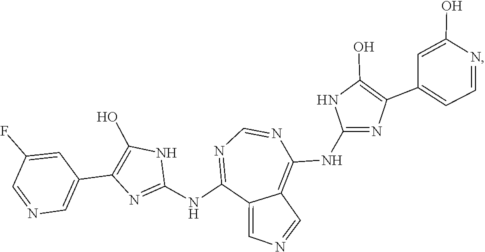 [SMILES: Oc1cc(-c2nc(Nc3ncnc(Nc4nc(-c5cncc(F)c5)c(O)[nH]4)c4cncc3-4)[nH]c2O)ccn1] | V-213 |
| 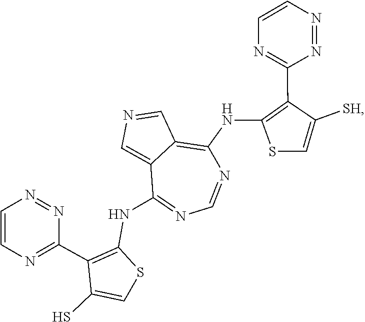 [SMILES: Sc1csc(Nc2ncnc(Nc3scc(S)c3-c3nccnn3)c3cncc2-3)c1-c1nccnn1] | V-214 |
| 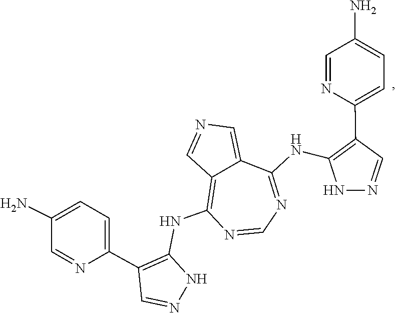 [SMILES: Nc1ccc(-c2cn[nH]c2Nc2ncnc(Nc3[nH]ncc3-c3ccc(N)cn3)c3cncc2-3)nc1] | V-215 |
| 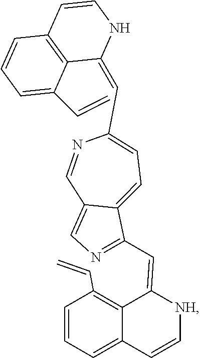 [SMILES: C=Cc1cccc2c1/C(=C\c1ccc3c(/C=C4/NC=Cc5cccc(C=C)c54)ncc-3cn1)NC=C2] | V-216 |
| 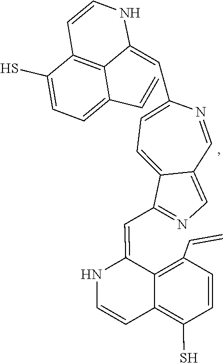 [SMILES: C=Cc1ccc(S)c2c1/C(=C\c1ccc3c(/C=C4/NC=Cc5c(S)ccc(C=C)c54)ncc-3cn1)NC=C2] | V-217 |
| 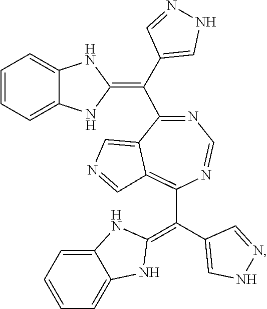 [SMILES: c1ccc2c(c1)NC(=C(c1cn[nH]c1)c1ncnc(C(=C3Nc4ccccc4N3)c3cn[nH]c3)c3cncc1-3)N2] | V-218 |
| 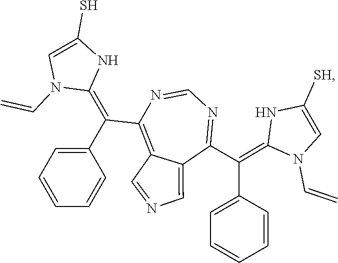 [SMILES: C=CN1C=C(S)N/C1=C(/c1ccccc1)c1ncnc(/C(=C2\NC(S)=CN2C=C)c2ccccc2)c2cncc1-2] | V-219 |
| 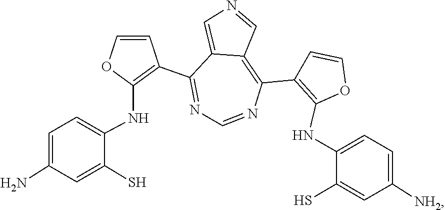 [SMILES: Nc1ccc(Nc2occc2-c2ncnc(-c3ccoc3Nc3ccc(N)cc3S)c3cncc2-3)c(S)c1] | V-220 |
| 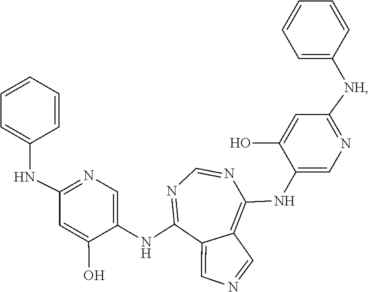 [SMILES: Oc1cc(Nc2ccccc2)ncc1Nc1ncnc(Nc2cnc(Nc3ccccc3)cc2O)c2cncc1-2] | V-221 |
| 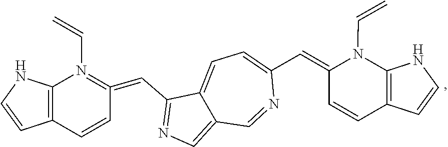 | V-222 |
| 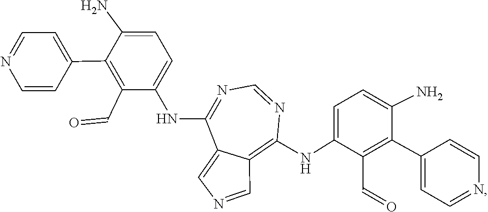 [SMILES: Nc1ccc(Nc2ncnc(Nc3ccc(N)c(-c4ccncc4)c3C=O)c3cncc2-3)c(C=O)c1-c1ccncc1] | V-223 |
| 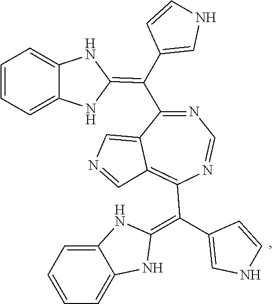 [SMILES: c1ccc2c(c1)NC(=C(c1cc[nH]c1)c1ncnc(C(=C3Nc4ccccc4N3)c3cc[nH]c3)c3cncc1-3)N2] | V-224 |
| 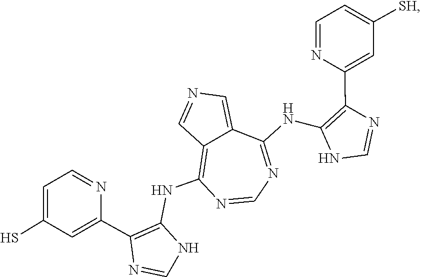 [SMILES: Sc1ccnc(-c2nc[nH]c2Nc2ncnc(Nc3[nH]cnc3-c3cc(S)ccn3)c3cncc2-3)c1] | V-225 |
| 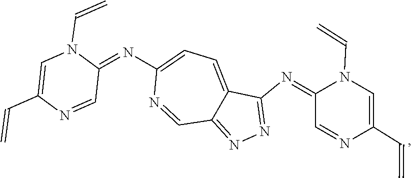 [SMILES: C=Cc1cn(C=C)/c(=N/c2ccc3c(/N=c4\cnc(C=C)cn4C=C)nnc-3cn2)cn1] | V-226 |
| 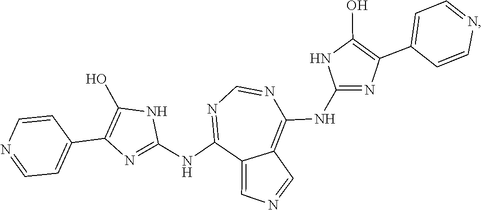 [SMILES: Oc1[nH]c(Nc2ncnc(Nc3nc(-c4ccncc4)c(O)[nH]3)c3cncc2-3)nc1-c1ccncc1] | V-227 |
| 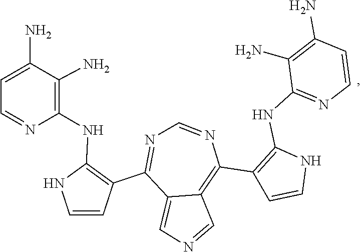 [SMILES: Nc1ccnc(Nc2[nH]ccc2-c2ncnc(-c3cc[nH]c3Nc3nccc(N)c3N)c3cncc2-3)c1N] | V-228 |
| 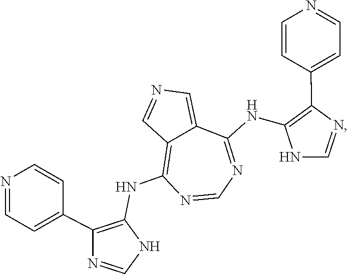 [SMILES: c1cc(-c2nc[nH]c2Nc2ncnc(Nc3[nH]cnc3-c3ccncc3)c3cncc2-3)ccn1] | V-229 |
| 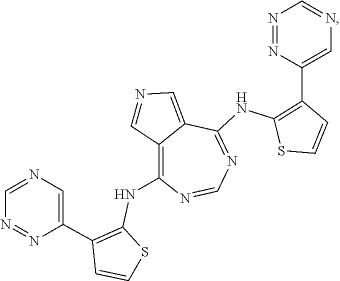 [SMILES: c1ncc(-c2ccsc2Nc2ncnc(Nc3sccc3-c3cncnn3)c3cncc2-3)nn1] | V-230 |
| 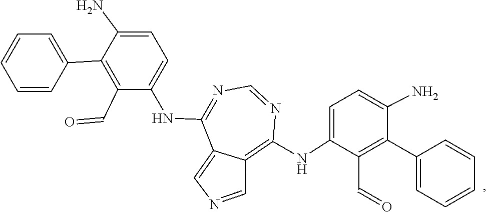 [SMILES: Nc1ccc(Nc2ncnc(Nc3ccc(N)c(-c4ccccc4)c3C=O)c3cncc2-3)c(C=O)c1-c1ccccc1] | V-231 |
| 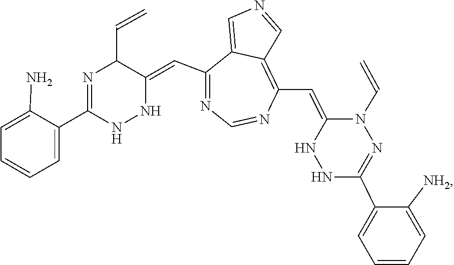 [SMILES: C=CC1N=C(c2ccccc2N)NN/C1=C\c1ncnc(/C=C2\NNC(c3ccccc3N)=NN2C=C)c2cncc1-2] | V-232 |
| 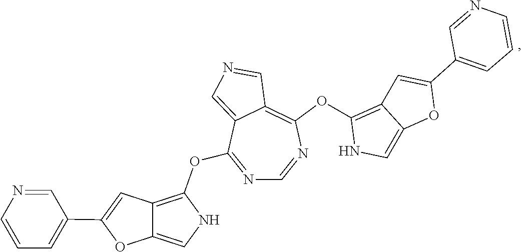 [SMILES: c1cncc(-c2cc3c(Oc4ncnc(Oc5[nH]cc6oc(-c7cccnc7)cc56)c5cncc4-5)[nH]cc3o2)c1] | V-233 |
| 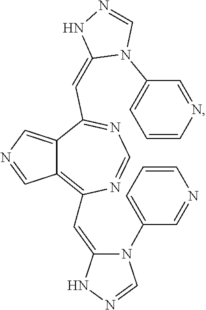 [SMILES: C1=NN/C(=C/c2ncnc(/C=C3/NN=CN3c3cccnc3)c3cncc2-3)N1c1cccnc1] | V-234 |
| 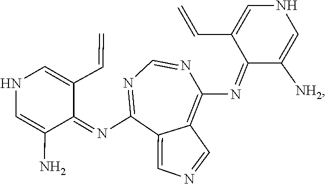 [SMILES: C=Cc1c[nH]cc(N)/c1=N/c1ncnc(/N=c2/c(N)c[nH]cc2C=C)c2cncc1-2] | V-235 |
| 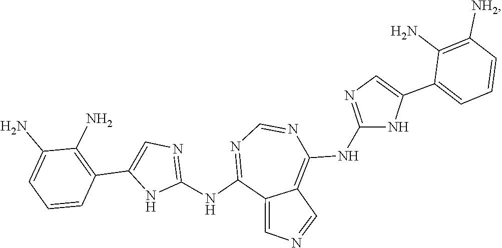 [SMILES: Nc1cccc(-c2cnc(Nc3ncnc(Nc4ncc(-c5cccc(N)c5N)[nH]4)c4cncc3-4)[nH]2)c1N] | V-236 |
| 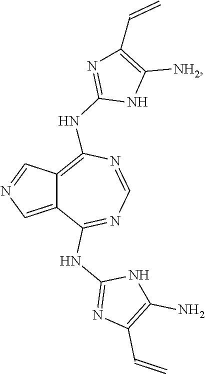 [SMILES: C=Cc1nc(Nc2ncnc(Nc3nc(C=C)c(N)[nH]3)c3cncc2-3)[nH]c1N] | V-237 |
| 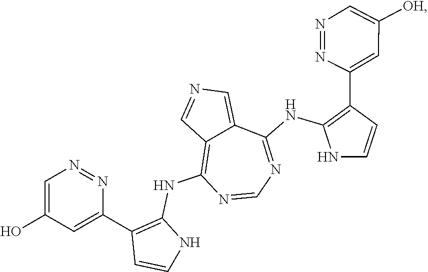 [SMILES: Oc1cnnc(-c2cc[nH]c2Nc2ncnc(Nc3[nH]ccc3-c3cc(O)cnn3)c3cncc2-3)c1] | V-238 |
| 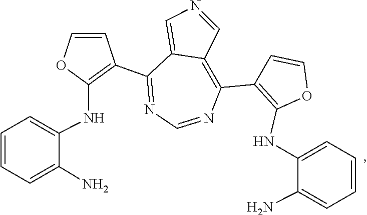 [SMILES: Nc1ccccc1Nc1occc1-c1ncnc(-c2ccoc2Nc2ccccc2N)c2cncc1-2] | V-239 |
| 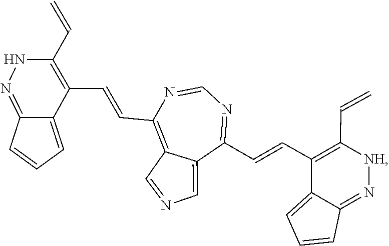 [SMILES: C=Cc1[nH]nc2cccc-2c1/C=C/c1ncnc(/C=C/c2c3cccc-3n[nH]c2C=C)c2cncc1-2] | V-240 |
| 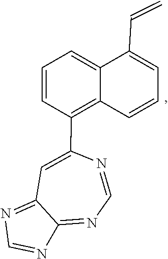 [SMILES: C=Cc1cccc2c(-c3cc4ncnc-4ncn3)cccc12] | V-241 |
| 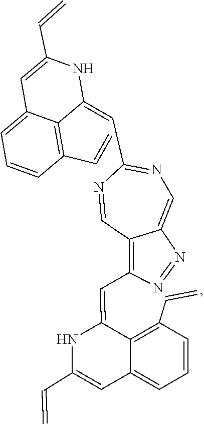 [SMILES: C=CC1=Cc2cccc(C=C)c2/C(=C\c2ncc3nnc(/C=C4/NC(C=C)=Cc5cccc(C=C)c54)c-3cn2)N1] | V-242 |
| 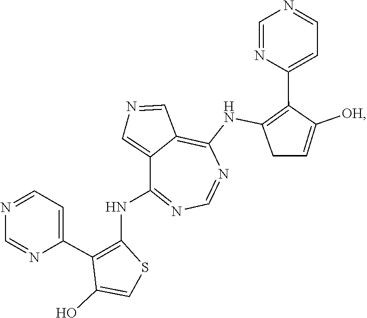 [SMILES: OC1=CCC(Nc2ncnc(Nc3scc(O)c3-c3ccncn3)c3cncc2-3)=C1c1ccncn1] | V-243 |
| 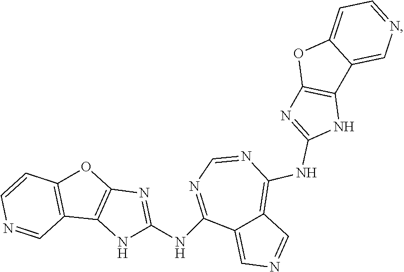 [SMILES: c1cc2oc3nc(Nc4ncnc(Nc5nc6oc7ccncc7c6[nH]5)c5cncc4-5)[nH]c3c2cn1] | V-244 |
| 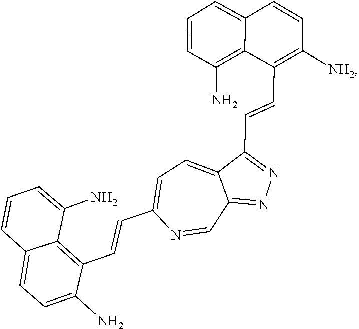 [SMILES: Nc1ccc2cccc(N)c2c1/C=C/c1ccc2c(/C=C/c3c(N)ccc4cccc(N)c34)nnc-2cn1] | V-245 |
| 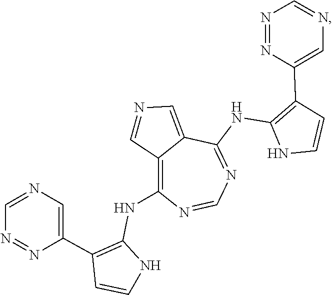 [SMILES: c1ncc(-c2cc[nH]c2Nc2ncnc(Nc3[nH]ccc3-c3cncnn3)c3cncc2-3)nn1] | V-246 |
| 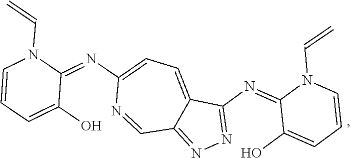 [SMILES: C=Cn1cccc(O)/c1=N\c1ccc2c(/N=c3\c(O)cccn3C=C)nnc-2cn1] | V-247 |
| 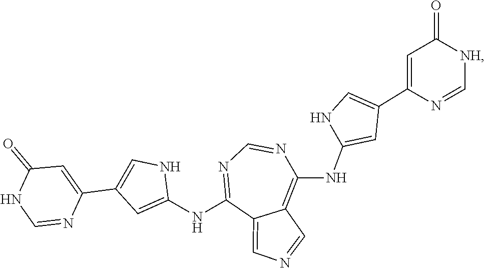 [SMILES: O=c1cc(-c2c[nH]c(Nc3ncnc(Nc4cc(-c5cc(=O)[nH]cn5)c[nH]4)c4cncc3-4)c2)nc[nH]1] | V-248 |
| 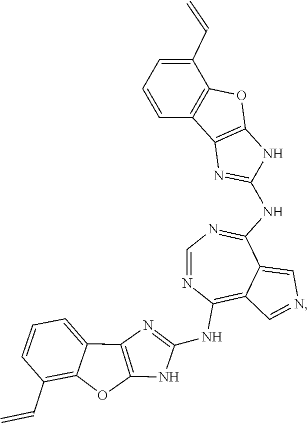 [SMILES: C=Cc1cccc2c1oc1[nH]c(Nc3ncnc(Nc4nc5c([nH]4)oc4c(C=C)cccc45)c4cncc3-4)nc12] | V-249 |
| 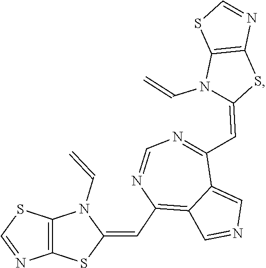 [SMILES: C=CN1/C(=C\c2ncnc(/C=C3/Sc4ncsc4N3C=C)c3cncc2-3)Sc2ncsc21] | V-250 |
| 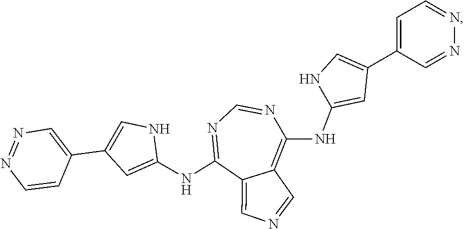 [SMILES: c1cc(-c2c[nH]c(Nc3ncnc(Nc4cc(-c5ccnnc5)c[nH]4)c4cncc3-4)c2)cnn1] | V-251 |
| 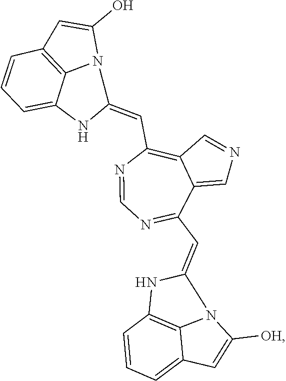 [SMILES: Oc1cc2cccc3[nH]/c(=C\c4ncnc(/C=c5\[nH]c6cccc7cc(O)n5c76)c5cncc4-5)n1c23] | V-252 |
| 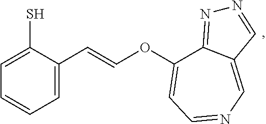 [SMILES: Sc1ccccc1/C=C/Oc1ccncc2cnnc1-2] | V-253 |
| 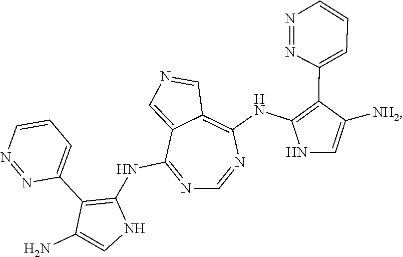 [SMILES: Nc1c[nH]c(Nc2ncnc(Nc3[nH]cc(N)c3-c3cccnn3)c3cncc2-3)c1-c1cccnn1] | V-254 |
| 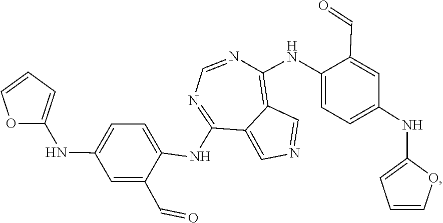 [SMILES: O=Cc1cc(Nc2ccco2)ccc1Nc1ncnc(Nc2ccc(Nc3ccco3)cc2C=O)c2cncc1-2] | V-255 |
| 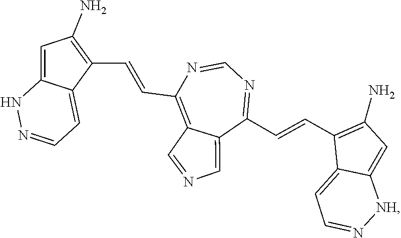 | V-256 |
| 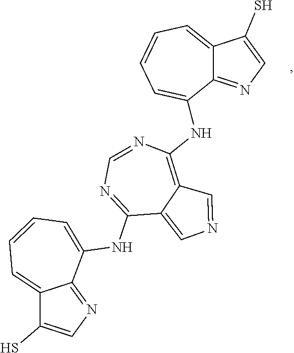 [SMILES: Sc1cnc2c(Nc3ncnc(Nc4ccccc5c(S)cnc4-5)c4cncc3-4)ccccc1-2] | V-257 |
| 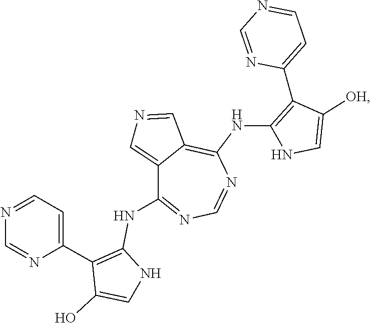 [SMILES: Oc1c[nH]c(Nc2ncnc(Nc3[nH]cc(O)c3-c3ccncn3)c3cncc2-3)c1-c1ccncn1] | V-258 |
| 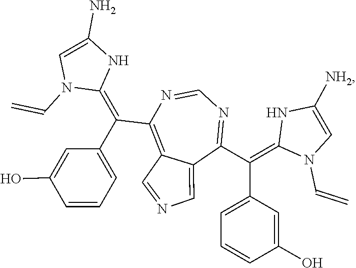 [SMILES: C=CN1C=C(N)N/C1=C(/c1cccc(O)c1)c1ncnc(/C(=C2\NC(N)=CN2C=C)c2cccc(O)c2)c2cncc1-2] | V-259 |
| 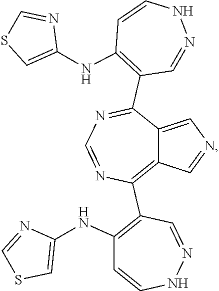 [SMILES: C1=CC(Nc2cscn2)=C(c2ncnc(C3=C(Nc4cscn4)C=CNN=C3)c3cncc2-3)C=NN1] | V-260 |
| 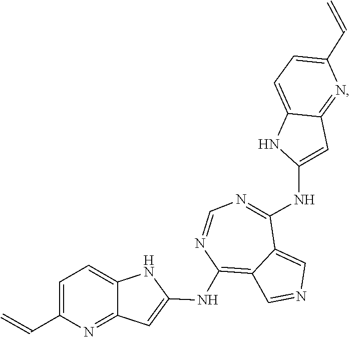 [SMILES: C=Cc1ccc2[nH]c(Nc3ncnc(Nc4cc5nc(C=C)ccc5[nH]4)c4cncc3-4)cc2n1] | V-261 |
| 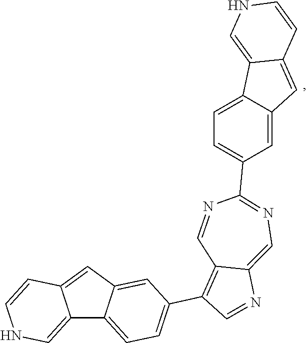 [SMILES: c1cc2cc3cc(-c4ncc5ncc(-c6ccc7c8c[nH]ccc-8cc7c6)c-5cn4)ccc3c-2c[nH]1] | V-262 |
| 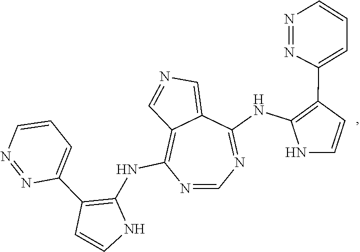 [SMILES: c1cnnc(-c2cc[nH]c2Nc2ncnc(Nc3[nH]ccc3-c3cccnn3)c3cncc2-3)c1] | V-263 |
| 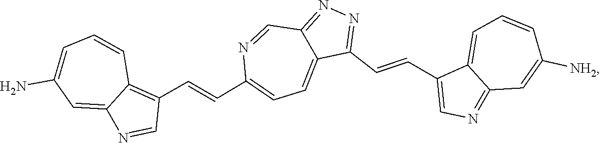 [SMILES: Nc1cccc2c(/C=C/c3ccc4c(/C=C/c5cnc6cc(N)cccc5-6)nnc-4cn3)cnc-2c1] | V-264 |
| 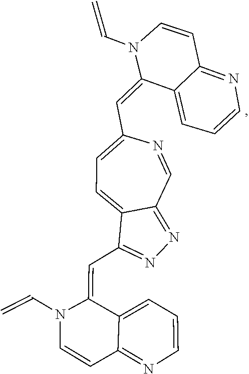 [SMILES: C=CN1C=Cc2ncccc2/C1=C\c1ccc2c(/C=C3\c4cccnc4C=CN3C=C)nnc-2cn1] | V-265 |
| 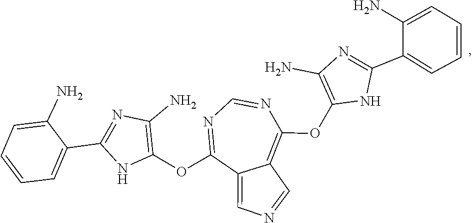 [SMILES: Nc1ccccc1-c1nc(N)c(Oc2ncnc(Oc3[nH]c(-c4ccccc4N)nc3N)c3cncc2-3)[nH]1] | V-266 |
| 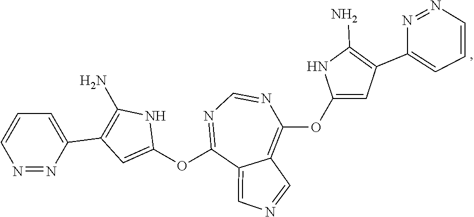 [SMILES: Nc1[nH]c(Oc2ncnc(Oc3cc(-c4cccnn4)c(N)[nH]3)c3cncc2-3)cc1-c1cccnn1] | V-267 |
| 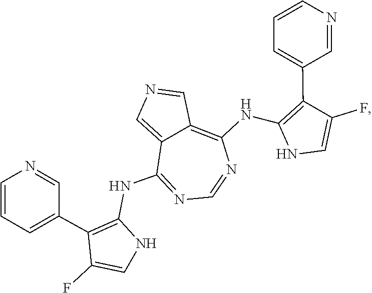 [SMILES: Fc1c[nH]c(Nc2ncnc(Nc3[nH]cc(F)c3-c3cccnc3)c3cncc2-3)c1-c1cccnc1] | V-268 |
| 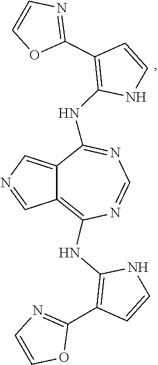 [SMILES: c1coc(-c2cc[nH]c2Nc2ncnc(Nc3[nH]ccc3-c3ncco3)c3cncc2-3)n1] | V-269 |
| 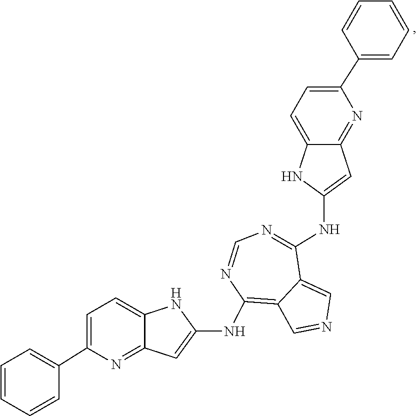 [SMILES: c1ccc(-c2ccc3[nH]c(Nc4ncnc(Nc5cc6nc(-c7ccccc7)ccc6[nH]5)c5cncc4-5)cc3n2)cc1] | V-270 |
| 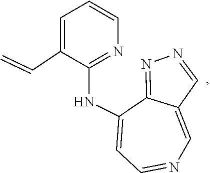 [SMILES: C=Cc1cccnc1Nc1ccncc2cnnc1-2] | V-271 |
| 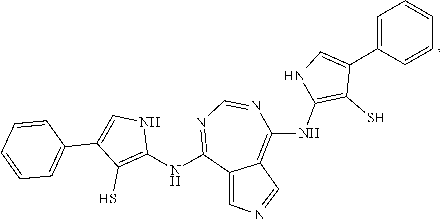 [SMILES: Sc1c(-c2ccccc2)c[nH]c1Nc1ncnc(Nc2[nH]cc(-c3ccccc3)c2S)c2cncc1-2] | V-272 |
| 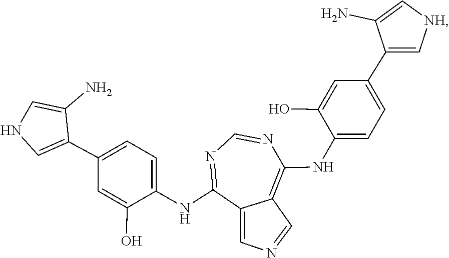 [SMILES: Nc1c[nH]cc1-c1ccc(Nc2ncnc(Nc3ccc(-c4c[nH]cc4N)cc3O)c3cncc2-3)c(O)c1] | V-273 |
| 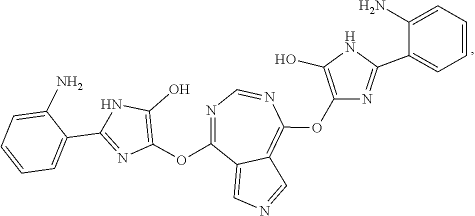 [SMILES: Nc1ccccc1-c1nc(Oc2ncnc(Oc3nc(-c4ccccc4N)[nH]c3O)c3cncc2-3)c(O)[nH]1] | V-274 |
| 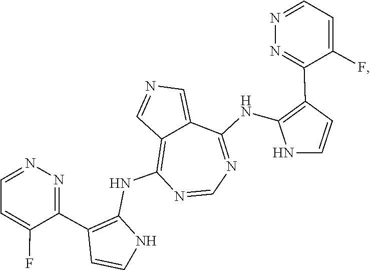 [SMILES: Fc1ccnnc1-c1cc[nH]c1Nc1ncnc(Nc2[nH]ccc2-c2nnccc2F)c2cncc1-2] | V-275 |
| 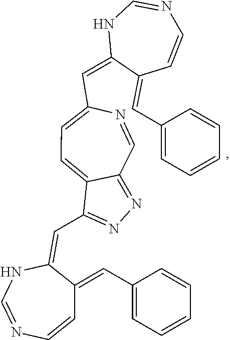 [SMILES: C1=CC(=C\c2ccccc2)/C(=C\c2ccc3c(/C=C4/NC=NC=C/C4=C\c4ccccc4)nnc-3cn2)NC=N1] | V-276 |
| 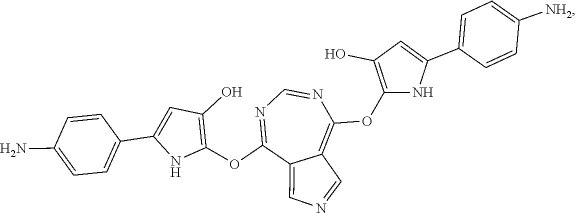 [SMILES: Nc1ccc(-c2cc(O)c(Oc3ncnc(Oc4[nH]c(-c5ccc(N)cc5)cc4O)c4cncc3-4)[nH]2)cc1] | V-277 |
| 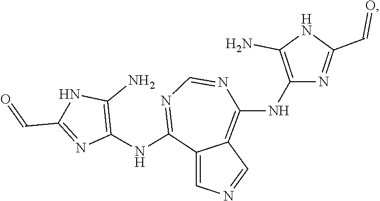 [SMILES: Nc1[nH]c(C=O)nc1Nc1ncnc(Nc2nc(C=O)[nH]c2N)c2cncc1-2] | V-278 |
| 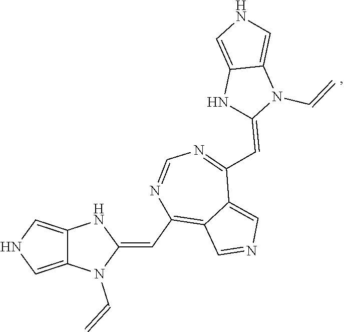 [SMILES: C=CN1/C(=C/c2ncnc(/C=C3\Nc4c[nH]cc4N3C=C)c3cncc2-3)Nc2c[nH]cc21] | V-279 |
| 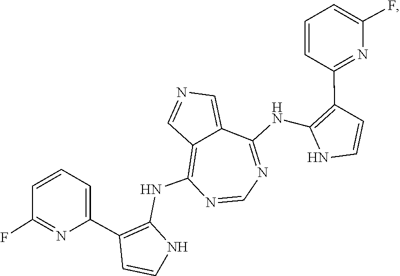 [SMILES: Fc1cccc(-c2cc[nH]c2Nc2ncnc(Nc3[nH]ccc3-c3cccc(F)n3)c3cncc2-3)n1] | V-280 |
| 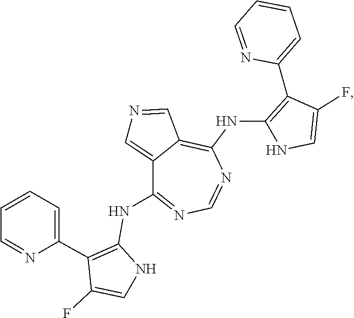 [SMILES: Fc1c[nH]c(Nc2ncnc(Nc3[nH]cc(F)c3-c3ccccn3)c3cncc2-3)c1-c1ccccn1] | V-281 |
| 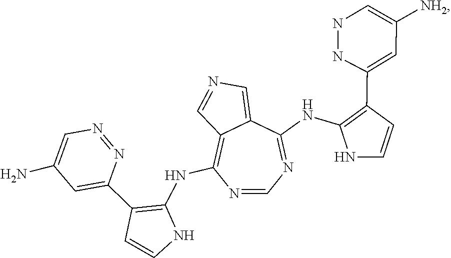 | V-282 |
| 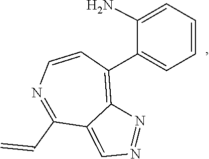 [SMILES: C=Cc1nccc(-c2ccccc2N)c2nncc1-2] | V-283 |
| 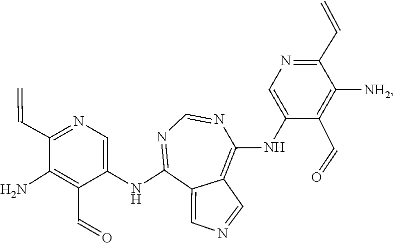 [SMILES: C=Cc1ncc(Nc2ncnc(Nc3cnc(C=C)c(N)c3C=O)c3cncc2-3)c(C=O)c1N] | V-284 |
| 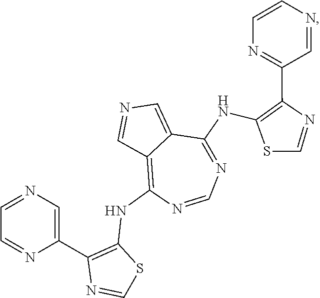 [SMILES: c1cnc(-c2ncsc2Nc2ncnc(Nc3scnc3-c3cnccn3)c3cncc2-3)cn1] | V-285 |
| 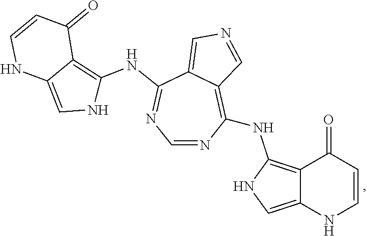 [SMILES: O=c1cc[nH]c2c[nH]c(Nc3ncnc(Nc4[nH]cc5[nH]ccc(=O)c45)c4cncc3-4)c12] | V-286 |
| 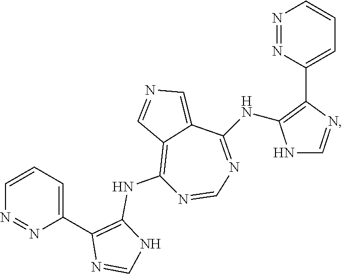 [SMILES: c1cnnc(-c2nc[nH]c2Nc2ncnc(Nc3[nH]cnc3-c3cccnn3)c3cncc2-3)c1] | V-287 |
| 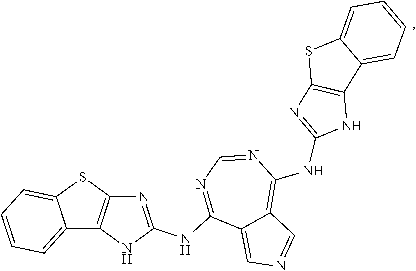 [SMILES: c1ccc2c(c1)sc1nc(Nc3ncnc(Nc4nc5sc6ccccc6c5[nH]4)c4cncc3-4)[nH]c12] | V-288 |
| 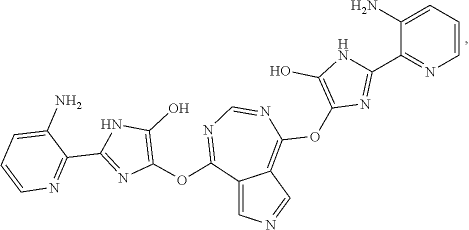 [SMILES: Nc1cccnc1-c1nc(Oc2ncnc(Oc3nc(-c4ncccc4N)[nH]c3O)c3cncc2-3)c(O)[nH]1] | V-289 |
| 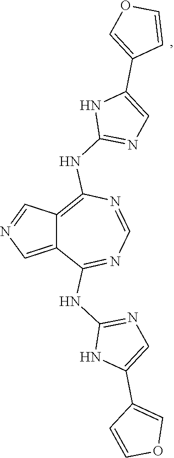 [SMILES: c1nc(Nc2ncc(-c3ccoc3)[nH]2)c2cncc-2c(Nc2ncc(-c3ccoc3)[nH]2)n1] | V-290 |
| 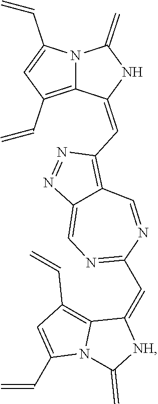 [SMILES: C=Cc1cc(C=C)n2c(=C)[nH]/c(=C/c3ncc4nnc(/C=c5/[nH]c(=C)n6c(C=C)cc(C=C)c56)c-4cn3)c12] | V-291 |
| 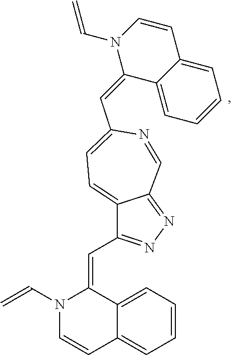 [SMILES: C=CN1C=Cc2ccccc2/C1=C\c1ccc2c(/C=C3\c4ccccc4C=CN3C=C)nnc-2cn1] | V-292 |
| 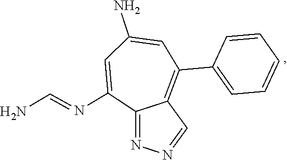 [SMILES: N/C=N/c1cc(N)cc(-c2ccccc2)c2cnnc1-2] | V-293 |
| 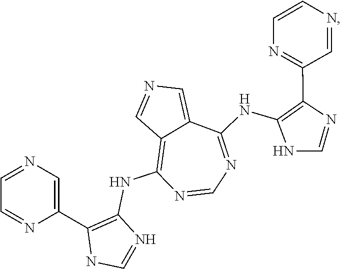 [SMILES: c1cnc(-c2nc[nH]c2Nc2ncnc(Nc3nc[nH]c3-c3cnccn3)c3cncc2-3)cn1] | V-294 |
| 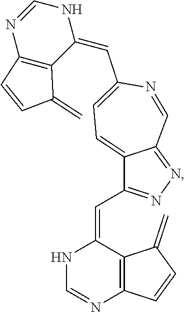 [SMILES: C=c1ccc2nc[nH]/c(=C/c3ccc4c(/C=c5/[nH]cnc6ccc(=C)c5=6)nnc-4cn3)c1=2] | V-295 |
| 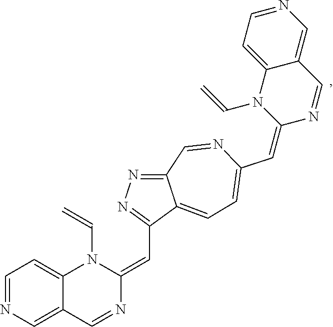 [SMILES: C=CN1/C(=C\c2ccc3c(/C=C4/N=Cc5cnccc5N4C=C)nnc-3cn2)N=Cc2cnccc21] | V-296 |
| 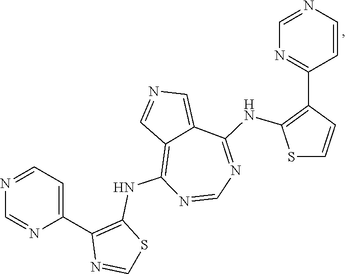 [SMILES: c1cc(-c2ccsc2Nc2ncnc(Nc3scnc3-c3ccncn3)c3cncc2-3)ncn1] | V-297 |
| 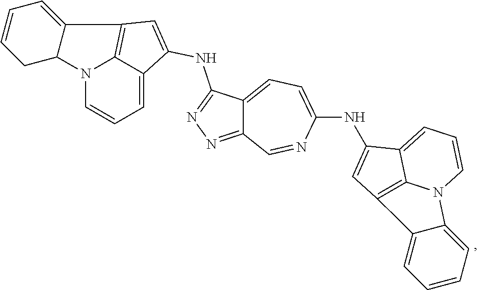 [SMILES: C1=CCC2C(=C1)c1cc(Nc3nnc4cnc(Nc5cc6c7ccccc7n7cccc5c67)ccc3-4)c3cccn2c1-3] | V-298 |
| 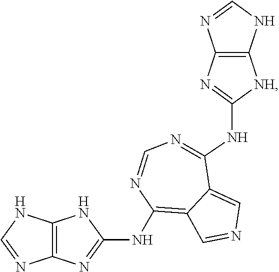 [SMILES: c1nc(Nc2nc3nc[nH]c3[nH]2)c2cncc-2c(Nc2nc3nc[nH]c3[nH]2)n1] | V-299 |
| 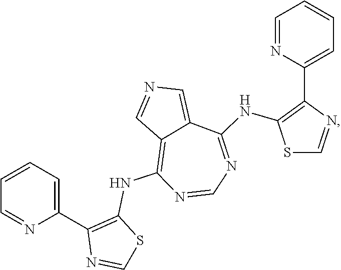 [SMILES: c1ccc(-c2ncsc2Nc2ncnc(Nc3scnc3-c3ccccn3)c3cncc2-3)nc1] | V-300 |
| 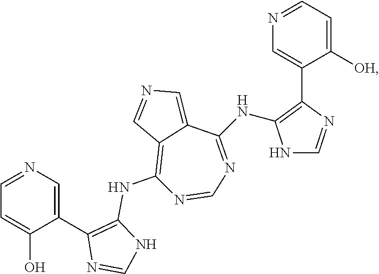 [SMILES: Oc1ccncc1-c1nc[nH]c1Nc1ncnc(Nc2[nH]cnc2-c2cnccc2O)c2cncc1-2] | V-301 |
| 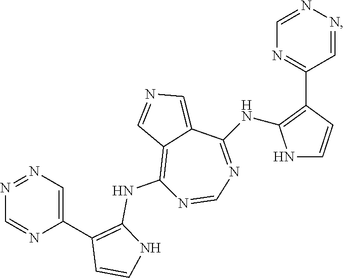 [SMILES: c1nncc(-c2cc[nH]c2Nc2ncnc(Nc3[nH]ccc3-c3cnncn3)c3cncc2-3)n1] | V-302 |
| 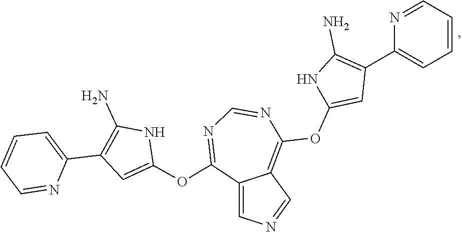 [SMILES: Nc1[nH]c(Oc2ncnc(Oc3cc(-c4ccccn4)c(N)[nH]3)c3cncc2-3)cc1-c1ccccn1] | V-303 |
| 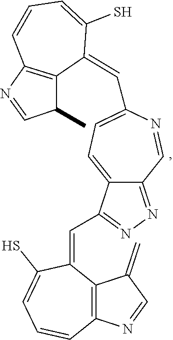 [SMILES: C=c1cnc2cccc(S)/c(=C/c3nnc4cnc(/C=C5/C(S)=CC=CC6=C5[C@@H](C)C=N6)ccc3-4)c1=2] | V-304 |
| 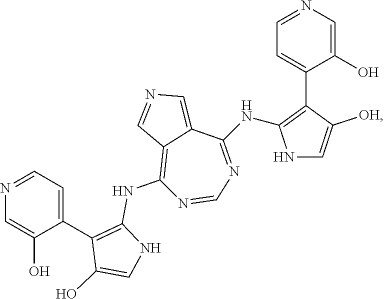 [SMILES: Oc1cnccc1-c1c(O)c[nH]c1Nc1ncnc(Nc2[nH]cc(O)c2-c2ccncc2O)c2cncc1-2] | V-305 |
| 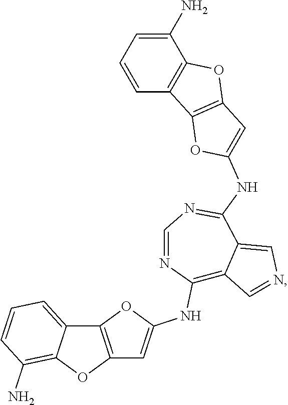 [SMILES: Nc1cccc2c1oc1cc(Nc3ncnc(Nc4cc5oc6c(N)cccc6c5o4)c4cncc3-4)oc12] | V-306 |
| 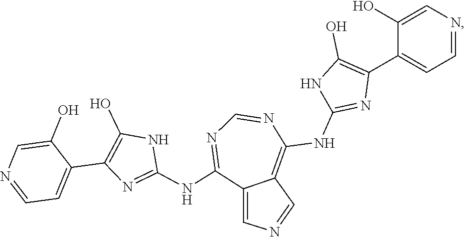 [SMILES: Oc1cnccc1-c1nc(Nc2ncnc(Nc3nc(-c4ccncc4O)c(O)[nH]3)c3cncc2-3)[nH]c1O] | V-307 |
| 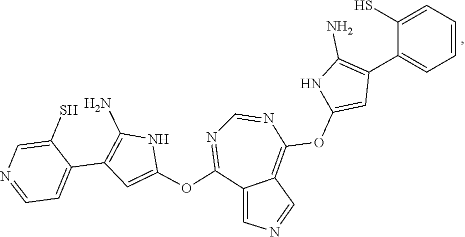 [SMILES: Nc1[nH]c(Oc2ncnc(Oc3cc(-c4ccncc4S)c(N)[nH]3)c3cncc2-3)cc1-c1ccccc1S] | V-308 |
| 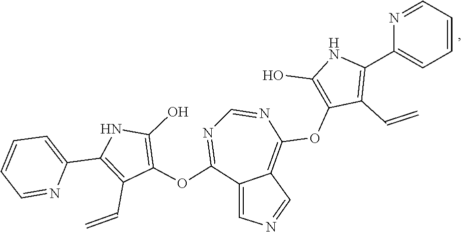 [SMILES: C=Cc1c(-c2ccccn2)[nH]c(O)c1Oc1ncnc(Oc2c(O)[nH]c(-c3ccccn3)c2C=C)c2cncc1-2] | V-309 |
| 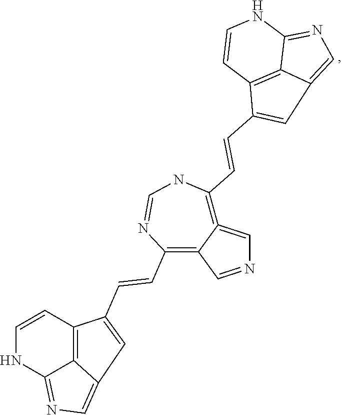 | V-310 |
| 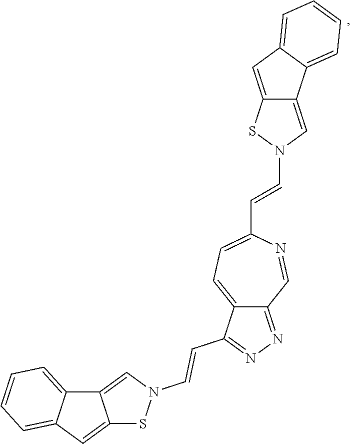 [SMILES: C(=C\n1cc2c3ccccc3cc-2s1)/c1ccc2c(/C=C/n3cc4c5ccccc5cc-4s3)nnc-2cn1] | V-311 |
| 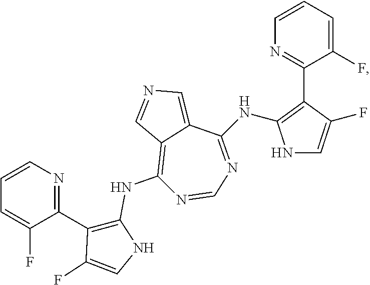 [SMILES: Fc1cccnc1-c1c(F)c[nH]c1Nc1ncnc(Nc2[nH]cc(F)c2-c2ncccc2F)c2cncc1-2] | V-312 |
| 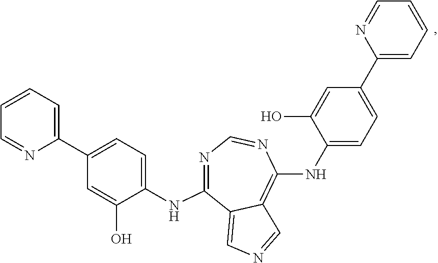 [SMILES: Oc1cc(-c2ccccn2)ccc1Nc1ncnc(Nc2ccc(-c3ccccn3)cc2O)c2cncc1-2] | V-313 |
| 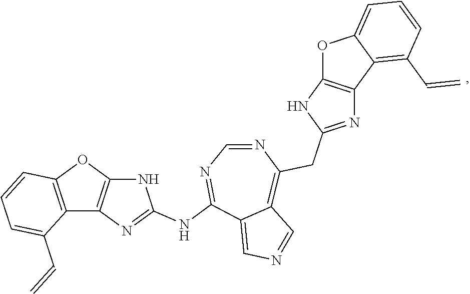 [SMILES: C=Cc1cccc2oc3[nH]c(Cc4ncnc(Nc5nc6c([nH]5)oc5cccc(C=C)c56)c5cncc4-5)nc3c12] | V-314 |
| 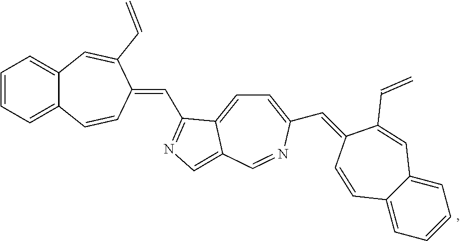 [SMILES: C=CC1=Cc2ccccc2C=C/C1=C\c1ccc2c(/C=C3\C=Cc4ccccc4C=C3C=C)ncc-2cn1] | V-315 |
| 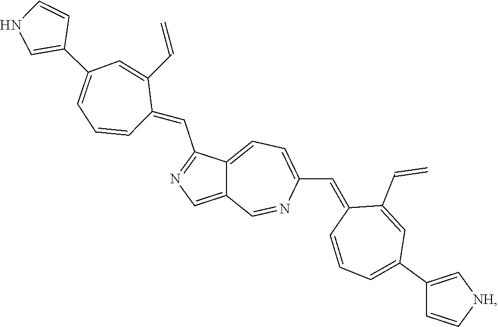 [SMILES: C=CC1=CC(c2cc[nH]c2)=CC=C/C1=C\c1ccc2c(/C=C3\C=CC=C(c4cc[nH]c4)C=C3C=C)ncc-2cn1] | V-316 |
| 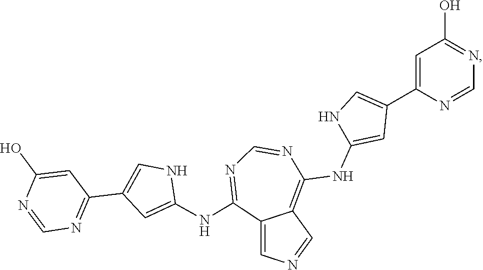 [SMILES: Oc1cc(-c2c[nH]c(Nc3ncnc(Nc4cc(-c5cc(O)ncn5)c[nH]4)c4cncc3-4)c2)ncn1] | V-317 |
| 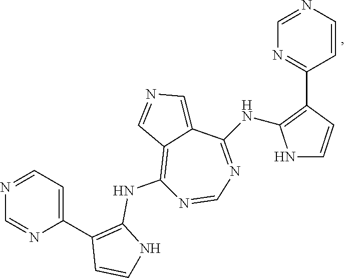 [SMILES: c1cc(-c2cc[nH]c2Nc2ncnc(Nc3[nH]ccc3-c3ccncn3)c3cncc2-3)ncn1] | V-318 |
| 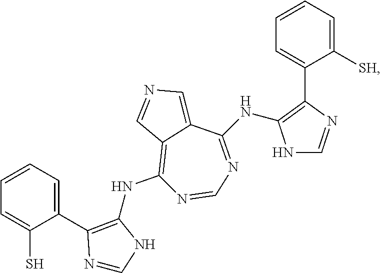 [SMILES: Sc1ccccc1-c1nc[nH]c1Nc1ncnc(Nc2[nH]cnc2-c2ccccc2S)c2cncc1-2] | V-319 |
| 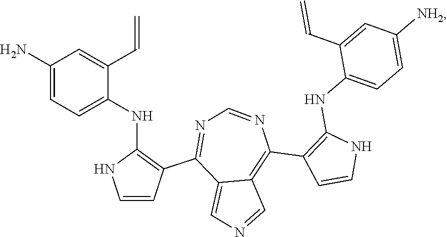 [SMILES: C=Cc1cc(N)ccc1Nc1[nH]ccc1-c1ncnc(-c2cc[nH]c2Nc2ccc(N)cc2C=C)c2cncc1-2] | V-320 |
| 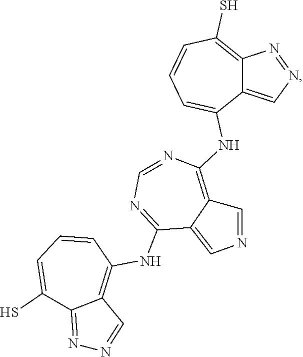 [SMILES: Sc1cccc(Nc2ncnc(Nc3cccc(S)c4nncc3-4)c3cncc2-3)c2cnnc1-2] | V-321 |
| 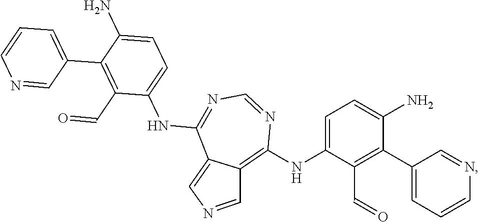 [SMILES: Nc1ccc(Nc2ncnc(Nc3ccc(N)c(-c4cccnc4)c3C=O)c3cncc2-3)c(C=O)c1-c1cccnc1] | V-322 |
| 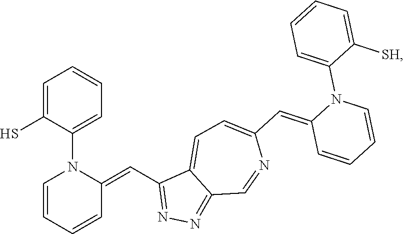 [SMILES: Sc1ccccc1N1C=CC=C/C1=C\c1ccc2c(/C=C3\C=CC=CN3c3ccccc3S)nnc-2cn1] | V-323 |
| 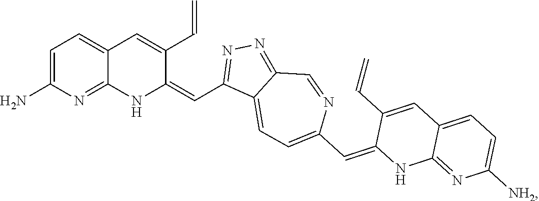 [SMILES: C=CC1=Cc2ccc(N)nc2N/C1=C/c1ccc2c(/C=C3/Nc4nc(N)ccc4C=C3C=C)nnc-2cn1] | V-324 |
| 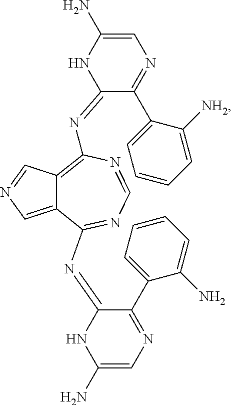 [SMILES: Nc1cnc(-c2ccccc2N)/c(=N\c2ncnc(/N=c3/[nH]c(N)cnc3-c3ccccc3N)c3cncc2-3)[nH]1] | V-325 |
| 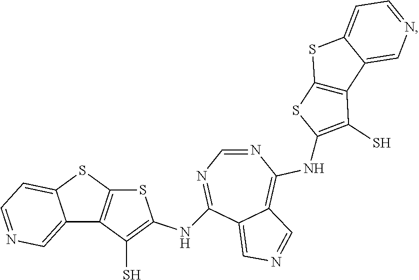 [SMILES: Sc1c(Nc2ncnc(Nc3sc4sc5ccncc5c4c3S)c3cncc2-3)sc2sc3ccncc3c12] | V-326 |
| 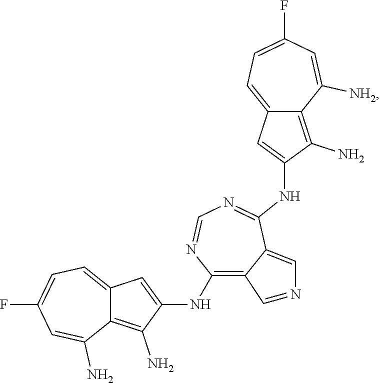 [SMILES: Nc1cc(F)ccc2cc(Nc3ncnc(Nc4cc5ccc(F)cc(N)c-5c4N)c4cncc3-4)c(N)c1-2] | V-327 |
| 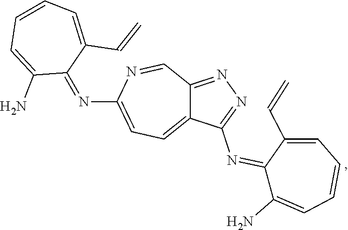 [SMILES: C=Cc1ccccc(N)/c1=N/c1ccc2c(/N=c3/c(N)ccccc3C=C)nnc-2cn1] | V-328 |
| 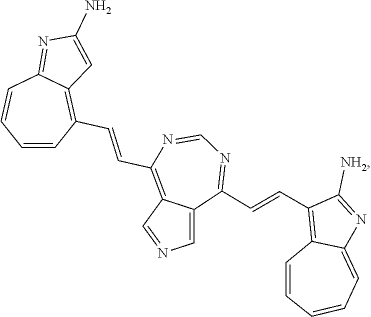 [SMILES: Nc1cc2c(/C=C/c3ncnc(/C=C/c4c5cccccc-5nc4N)c4cncc3-4)ccccc-2n1] | V-329 |
| 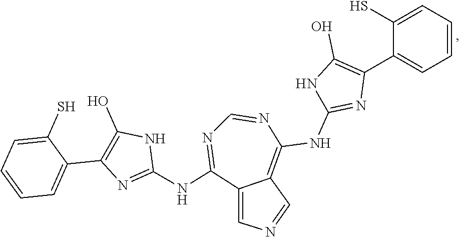 [SMILES: Oc1[nH]c(Nc2ncnc(Nc3nc(-c4ccccc4S)c(O)[nH]3)c3cncc2-3)nc1-c1ccccc1S] | V-330 |
| 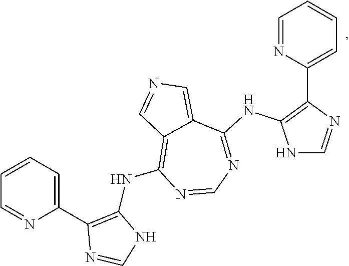 [SMILES: c1ccc(-c2nc[nH]c2Nc2ncnc(Nc3[nH]cnc3-c3ccccn3)c3cncc2-3)nc1] | V-331 |
| 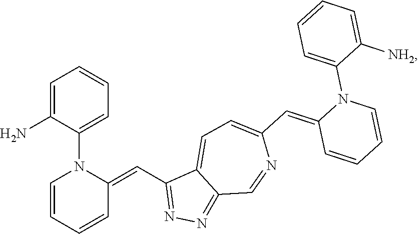 [SMILES: Nc1ccccc1N1C=CC=C/C1=C\c1ccc2c(/C=C3\C=CC=CN3c3ccccc3N)nnc-2cn1] | V-332 |
| 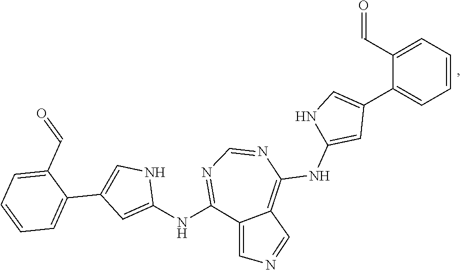 [SMILES: O=Cc1ccccc1-c1c[nH]c(Nc2ncnc(Nc3cc(-c4ccccc4C=O)c[nH]3)c3cncc2-3)c1] | V-333 |
| 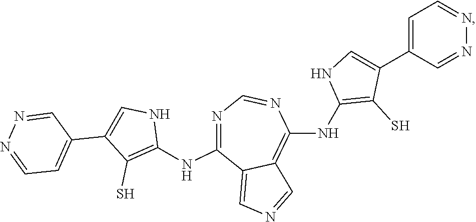 [SMILES: Sc1c(-c2ccnnc2)c[nH]c1Nc1ncnc(Nc2[nH]cc(-c3ccnnc3)c2S)c2cncc1-2] | V-334 |
| 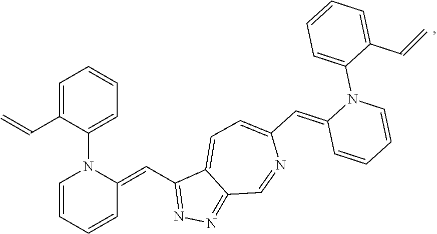 [SMILES: C=Cc1ccccc1N1C=CC=C/C1=C\c1ccc2c(/C=C3\C=CC=CN3c3ccccc3C=C)nnc-2cn1] | V-335 |
| 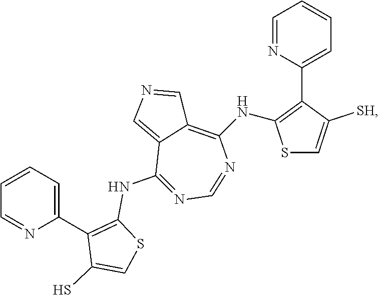 [SMILES: Sc1csc(Nc2ncnc(Nc3scc(S)c3-c3ccccn3)c3cncc2-3)c1-c1ccccn1] | V-336 |
| 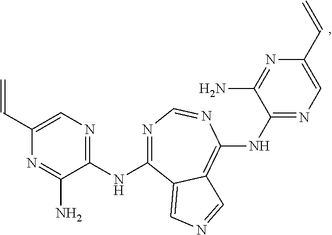 [SMILES: C=Cc1cnc(Nc2ncnc(Nc3ncc(C=C)nc3N)c3cncc2-3)c(N)n1] | V-337 |
| 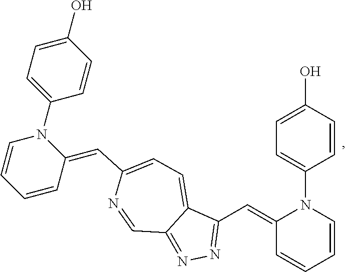 [SMILES: Oc1ccc(N2C=CC=C/C2=C\c2ccc3c(/C=C4\C=CC=CN4c4ccc(O)cc4)nnc-3cn2)cc1] | V-338 |
| 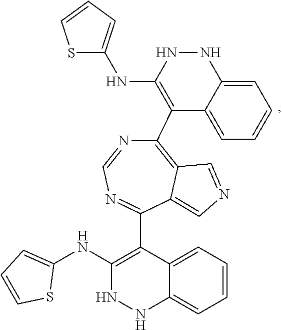 [SMILES: c1csc(NC2=C(c3ncnc(C4=C(Nc5cccs5)NNc5ccccc54)c4cncc3-4)c3ccccc3NN2)c1] | V-339 |
| 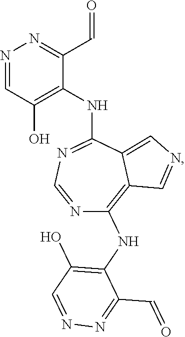 [SMILES: O=Cc1nncc(O)c1Nc1ncnc(Nc2c(O)cnnc2C=O)c2cncc1-2] | V-340 |
| 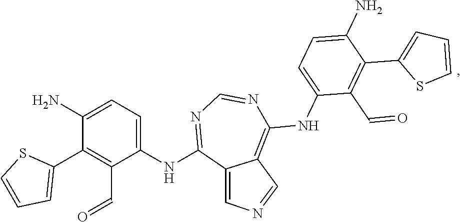 [SMILES: Nc1ccc(Nc2ncnc(Nc3ccc(N)c(-c4cccs4)c3C=O)c3cncc2-3)c(C=O)c1-c1cccs1] | V-341 |
| 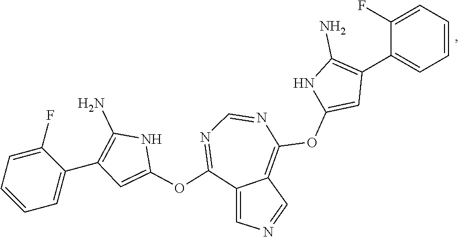 [SMILES: Nc1[nH]c(Oc2ncnc(Oc3cc(-c4ccccc4F)c(N)[nH]3)c3cncc2-3)cc1-c1ccccc1F] | V-342 |
| 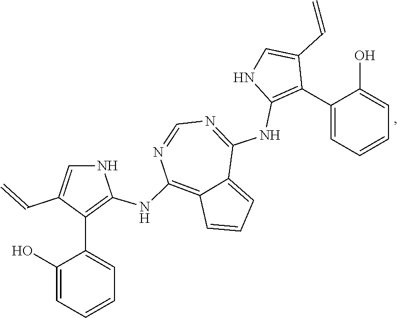 [SMILES: C=Cc1c[nH]c(Nc2ncnc(Nc3[nH]cc(C=C)c3-c3ccccc3O)c3cccc2-3)c1-c1ccccc1O] | V-343 |
| 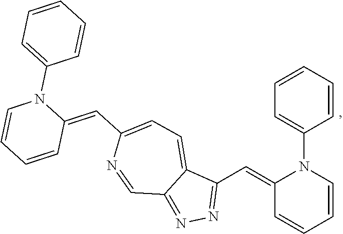 [SMILES: C1=C/C(=C\c2ccc3c(/C=C4\C=CC=CN4c4ccccc4)nnc-3cn2)N(c2ccccc2)C=C1] | V-344 |
| 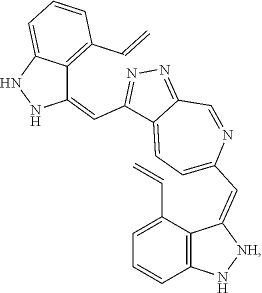 [SMILES: C=Cc1cccc2c1/C(=C\c1ccc3c(/C=C4/NNc5cccc(C=C)c54)nnc-3cn1)NN2] | V-345 |
| 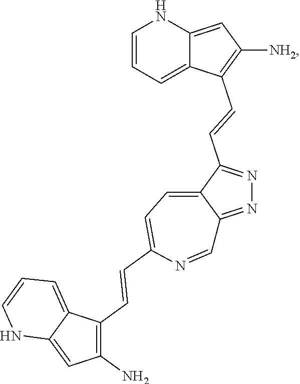 [SMILES: Nc1cc2[nH]cccc-2c1/C=C/c1ccc2c(/C=C/c3c(N)cc4[nH]cccc3-4)nnc-2cn1] | V-346 |
| 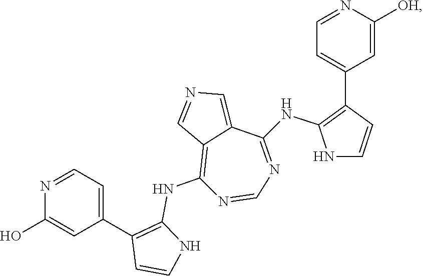 [SMILES: Oc1cc(-c2cc[nH]c2Nc2ncnc(Nc3[nH]ccc3-c3ccnc(O)c3)c3cncc2-3)ccn1] | V-347 |
| 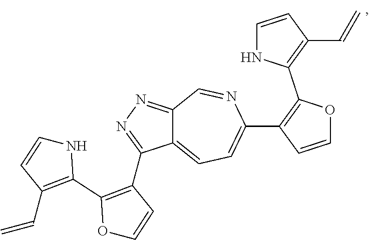 [SMILES: C=Cc1cc[nH]c1-c1occc1-c1ccc2c(-c3ccoc3-c3[nH]ccc3C=C)nnc-2cn1] | V-348 |
| 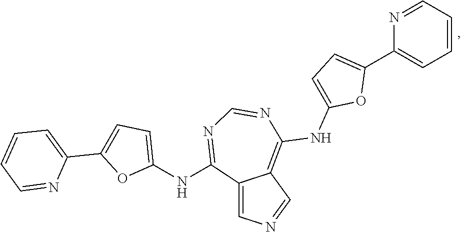 [SMILES: c1ccc(-c2ccc(Nc3ncnc(Nc4ccc(-c5ccccn5)o4)c4cncc3-4)o2)nc1] | V-349 |
| 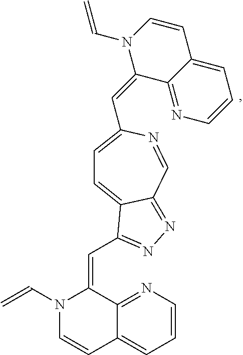 [SMILES: C=CN1C=Cc2cccnc2/C1=C\c1ccc2c(/C=C3\c4ncccc4C=CN3C=C)nnc-2cn1] | V-350 |
| 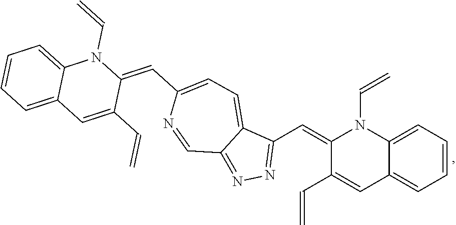 [SMILES: C=CC1=Cc2ccccc2N(C=C)/C1=C/c1ccc2c(/C=C3\C(C=C)=Cc4ccccc4N3C=C)nnc-2cn1] | V-351 |
| 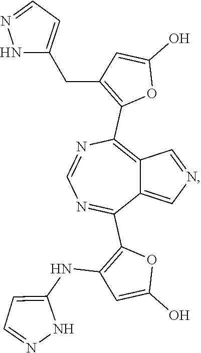 [SMILES: Oc1cc(Cc2ccn[nH]2)c(-c2ncnc(-c3oc(O)cc3Nc3ccn[nH]3)c3cncc2-3)o1] | V-352 |
| 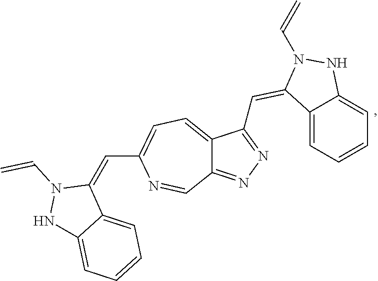 [SMILES: C=CN1Nc2ccccc2/C1=C\c1ccc2c(/C=C3\c4ccccc4NN3C=C)nnc-2cn1] | V-353 |
| 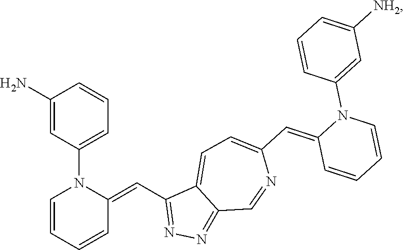 [SMILES: Nc1cccc(N2C=CC=C/C2=C\c2ccc3c(/C=C4\C=CC=CN4c4cccc(N)c4)nnc-3cn2)c1] | V-354 |
| 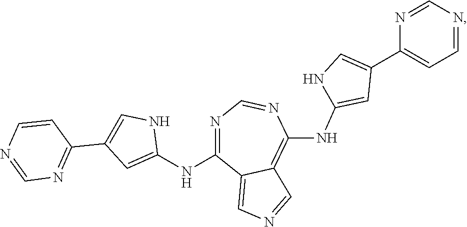 [SMILES: c1cc(-c2c[nH]c(Nc3ncnc(Nc4cc(-c5ccncn5)c[nH]4)c4cncc3-4)c2)ncn1] | V-355 |
| 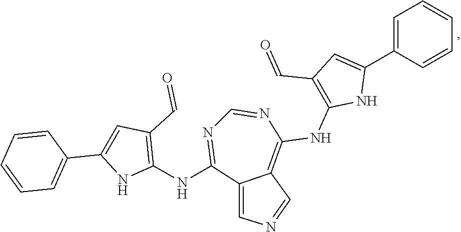 [SMILES: O=Cc1cc(-c2ccccc2)[nH]c1Nc1ncnc(Nc2[nH]c(-c3ccccc3)cc2C=O)c2cncc1-2] | V-356 |
| 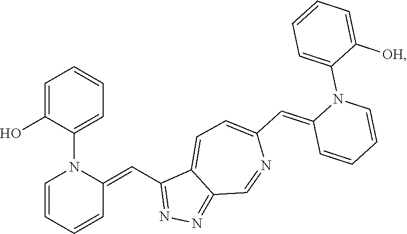 [SMILES: Oc1ccccc1N1C=CC=C/C1=C\c1ccc2c(/C=C3\C=CC=CN3c3ccccc3O)nnc-2cn1] | V-357 |
| 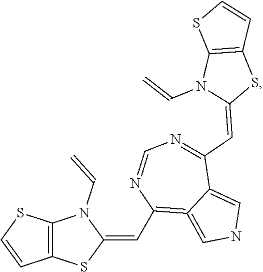 | V-358 |
| 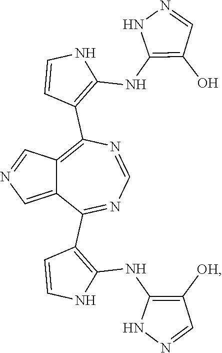 [SMILES: Oc1cn[nH]c1Nc1[nH]ccc1-c1ncnc(-c2cc[nH]c2Nc2[nH]ncc2O)c2cncc1-2] | V-359 |
| 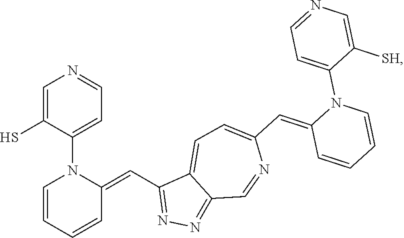 [SMILES: Sc1cnccc1N1C=CC=C/C1=C\c1ccc2c(/C=C3\C=CC=CN3c3ccncc3S)nnc-2cn1] | V-360 |
| 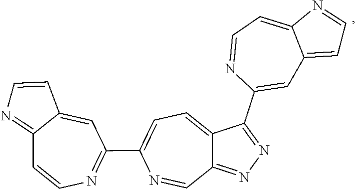 [SMILES: c1cc2cc(-c3ccc4c(-c5cc6ccnc-6ccn5)nnc-4cn3)nccc-2n1] | V-361 |
| 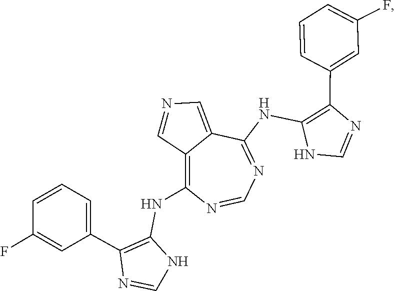 [SMILES: Fc1cccc(-c2nc[nH]c2Nc2ncnc(Nc3[nH]cnc3-c3cccc(F)c3)c3cncc2-3)c1] | V-362 |
| 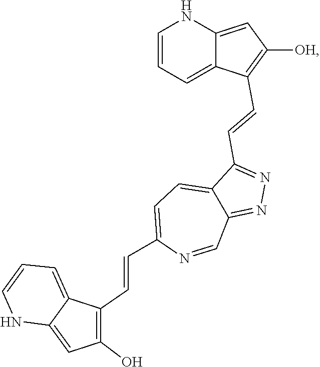 [SMILES: Oc1cc2[nH]cccc-2c1/C=C/c1ccc2c(/C=C/c3c(O)cc4[nH]cccc3-4)nnc-2cn1] | V-363 |
| 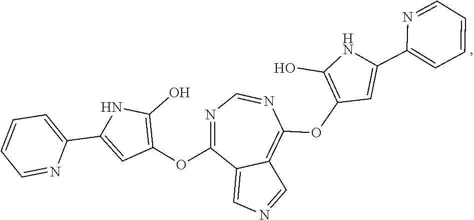 [SMILES: Oc1[nH]c(-c2ccccn2)cc1Oc1ncnc(Oc2cc(-c3ccccn3)[nH]c2O)c2cncc1-2] | V-364 |
| 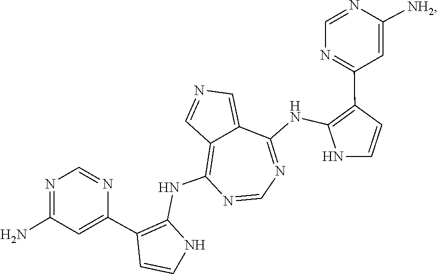 [SMILES: Nc1cc(-c2cc[nH]c2Nc2ncnc(Nc3[nH]ccc3-c3cc(N)ncn3)c3cncc2-3)ncn1] | V-365 |
| 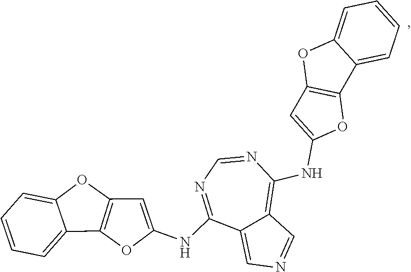 [SMILES: c1ccc2c(c1)oc1cc(Nc3ncnc(Nc4cc5oc6ccccc6c5o4)c4cncc3-4)oc12] | V-366 |
| 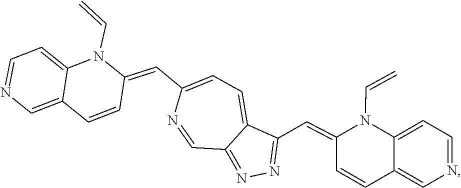 [SMILES: C=CN1/C(=C/c2ccc3c(/C=C4\C=Cc5cnccc5N4C=C)nnc-3cn2)C=Cc2cnccc21] | V-367 |
| 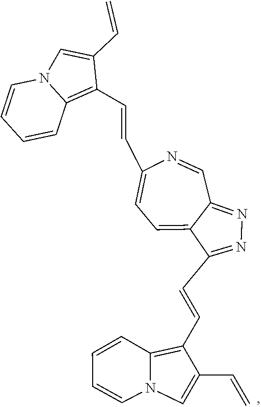 [SMILES: C=Cc1cn2ccccc2c1/C=C/c1ccc2c(/C=C/c3c(C=C)cn4ccccc34)nnc-2cn1] | V-368 |
| 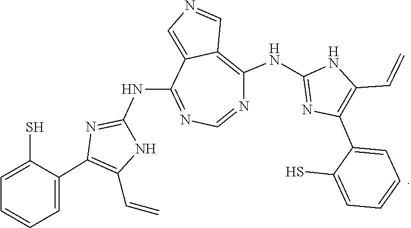 [SMILES: C=Cc1[nH]c(Nc2ncnc(Nc3nc(-c4ccccc4S)c(C=C)[nH]3)c3cncc2-3)nc1-c1ccccc1S] | V-369 |
| 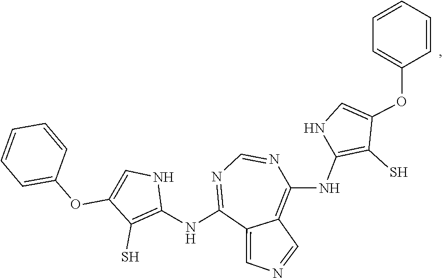 [SMILES: Sc1c(Oc2ccccc2)c[nH]c1Nc1ncnc(Nc2[nH]cc(Oc3ccccc3)c2S)c2cncc1-2] | V-370 |
| 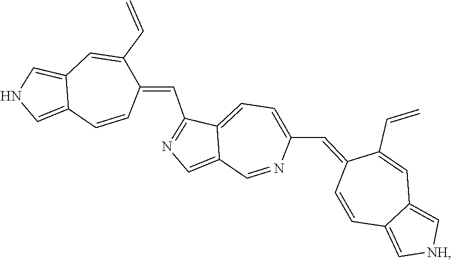 [SMILES: C=CC1=Cc2c[nH]cc2C=C/C1=C\c1ccc2c(/C=C3\C=Cc4c[nH]cc4C=C3C=C)ncc-2cn1] | V-371 |
| 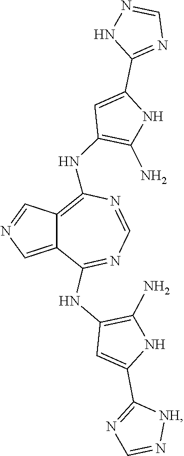 [SMILES: Nc1[nH]c(-c2ncn[nH]2)cc1Nc1ncnc(Nc2cc(-c3ncn[nH]3)[nH]c2N)c2cncc1-2] | V-372 |
| 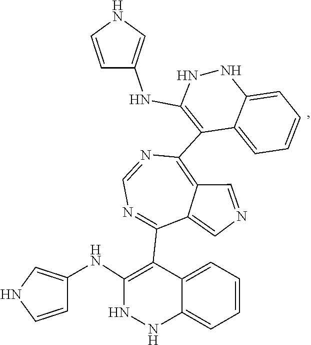 [SMILES: c1ccc2c(c1)NNC(Nc1cc[nH]c1)=C2c1ncnc(C2=C(Nc3cc[nH]c3)NNc3ccccc32)c2cncc1-2] | V-373 |
| 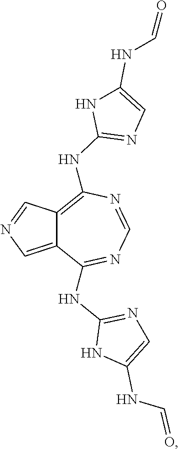 [SMILES: O=CNc1cnc(Nc2ncnc(Nc3ncc(NC=O)[nH]3)c3cncc2-3)[nH]1] | V-374 |
| 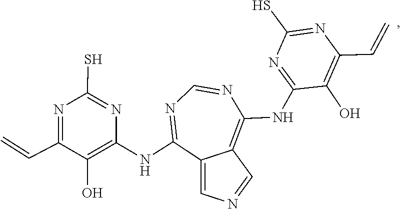 [SMILES: C=Cc1nc(S)nc(Nc2ncnc(Nc3nc(S)nc(C=C)c3O)c3cncc2-3)c1O] | V-375 |
| 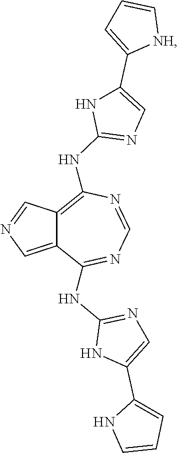 [SMILES: c1c[nH]c(-c2cnc(Nc3ncnc(Nc4ncc(-c5ccc[nH]5)[nH]4)c4cncc3-4)[nH]2)c1] | V-376 |
| 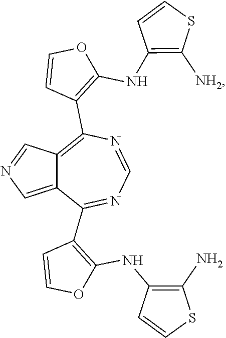 [SMILES: Nc1sccc1Nc1occc1-c1ncnc(-c2ccoc2Nc2ccsc2N)c2cncc1-2] | V-377 |
| 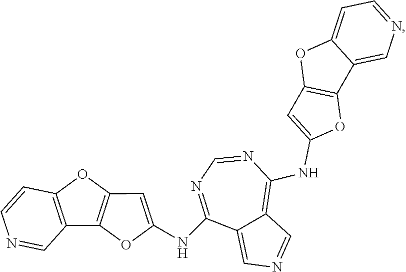 [SMILES: c1cc2oc3cc(Nc4ncnc(Nc5cc6oc7ccncc7c6o5)c5cncc4-5)oc3c2cn1] | V-378 |
| 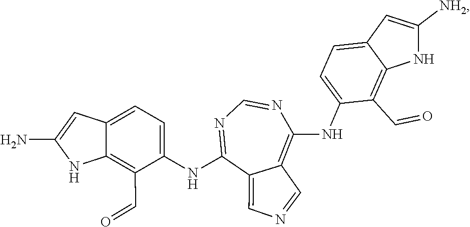 [SMILES: Nc1cc2ccc(Nc3ncnc(Nc4ccc5cc(N)[nH]c5c4C=O)c4cncc3-4)c(C=O)c2[nH]1] | V-379 |
| 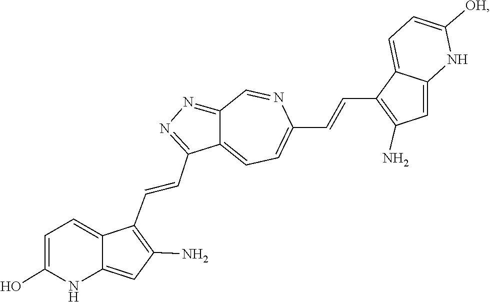 | V-380 |
| 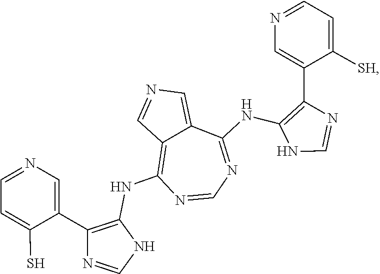 [SMILES: Sc1ccncc1-c1nc[nH]c1Nc1ncnc(Nc2[nH]cnc2-c2cnccc2S)c2cncc1-2] | V-381 |
| 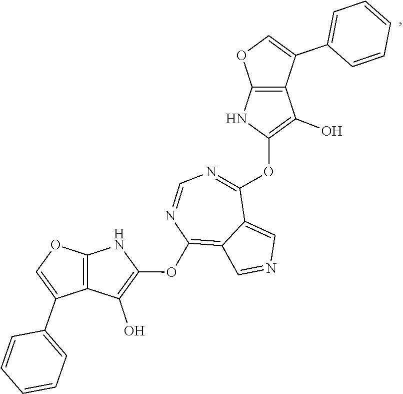 [SMILES: Oc1c(Oc2ncnc(Oc3[nH]c4occ(-c5ccccc5)c4c3O)c3cncc2-3)[nH]c2occ(-c3ccccc3)c12] | V-382 |
| 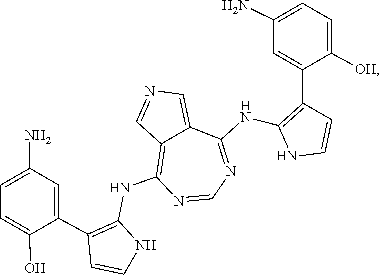 [SMILES: Nc1ccc(O)c(-c2cc[nH]c2Nc2ncnc(Nc3[nH]ccc3-c3cc(N)ccc3O)c3cncc2-3)c1] | V-383 |
| 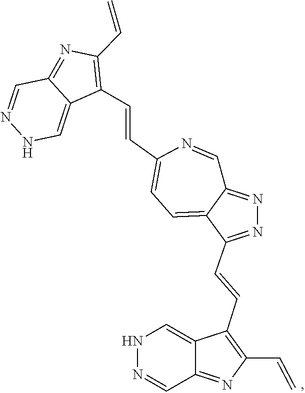 [SMILES: C=Cc1nc2cn[nH]cc-2c1/C=C/c1ccc2c(/C=C/c3c4c[nH]ncc-4nc3C=C)nnc-2cn1] | V-384 |
| 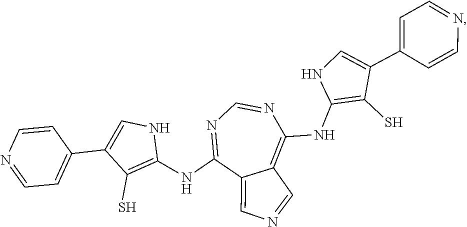 [SMILES: Sc1c(-c2ccncc2)c[nH]c1Nc1ncnc(Nc2[nH]cc(-c3ccncc3)c2S)c2cncc1-2] | V-385 |
| 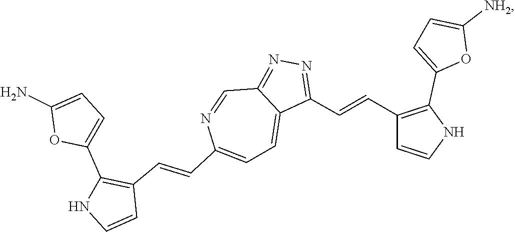 [SMILES: Nc1ccc(-c2[nH]ccc2/C=C/c2ccc3c(/C=C/c4cc[nH]c4-c4ccc(N)o4)nnc-3cn2)o1] | V-386 |
| 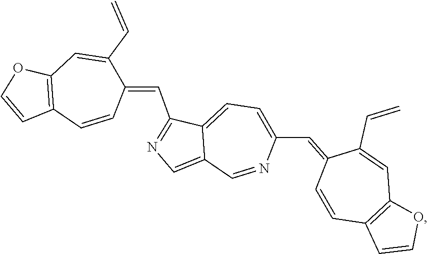 [SMILES: C=CC1=Cc2occc2C=C/C1=C\c1ccc2c(/C=C3\C=Cc4ccoc4C=C3C=C)ncc-2cn1] | V-387 |
| 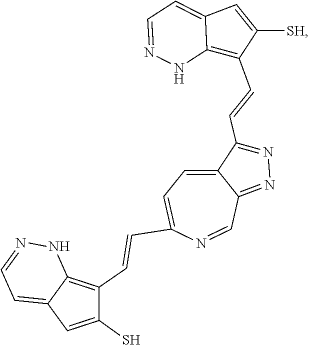 [SMILES: Sc1cc2ccn[nH]c-2c1/C=C/c1ccc2c(/C=C/c3c(S)cc4ccn[nH]c3-4)nnc-2cn1] | V-388 |
| 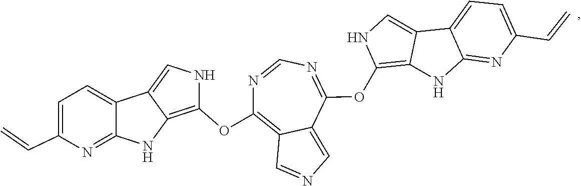 [SMILES: C=Cc1ccc2c(n1)[nH]c1c(Oc3ncnc(Oc4[nH]cc5c4[nH]c4nc(C=C)ccc45)c4cncc3-4)[nH]cc12] | V-389 |
| 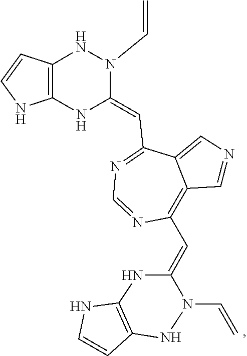 | V-390 |
| 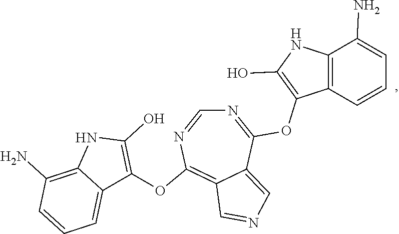 [SMILES: Nc1cccc2c(Oc3ncnc(Oc4c(O)[nH]c5c(N)cccc45)c4cncc3-4)c(O)[nH]c12] | V-391 |
| 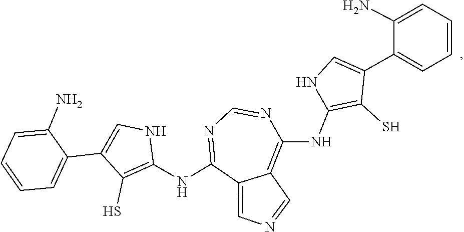 [SMILES: Nc1ccccc1-c1c[nH]c(Nc2ncnc(Nc3[nH]cc(-c4ccccc4N)c3S)c3cncc2-3)c1S] | V-392 |
| 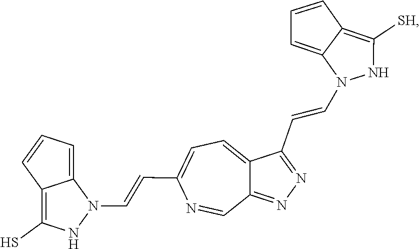 [SMILES: Sc1[nH]n(/C=C/c2ccc3c(/C=C/n4[nH]c(S)c5cccc4-5)nnc-3cn2)c2cccc1-2] | V-393 |
| 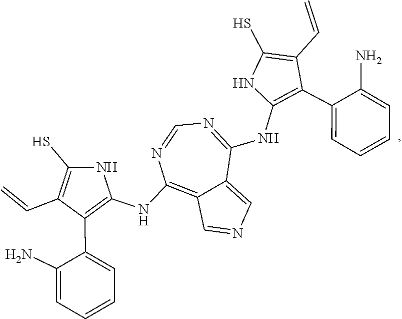 [SMILES: C=Cc1c(S)[nH]c(Nc2ncnc(Nc3[nH]c(S)c(C=C)c3-c3ccccc3N)c3cncc2-3)c1-c1ccccc1N] | V-394 |
| 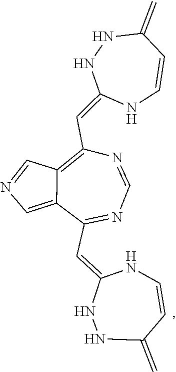 [SMILES: C=c1cc[nH]/c(=C\c2ncnc(/C=c3\[nH]ccc(=C)[nH][nH]3)c3cncc2-3)[nH][nH]1] | V-395 |
| 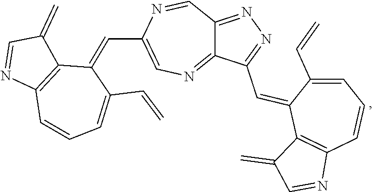 [SMILES: C=Cc1cccc2ncc(=C)c=2/c1=C/c1cnc2c(/C=c3\c(C=C)cccc4ncc(=C)c3=4)nnc-2cn1] | V-396 |
| 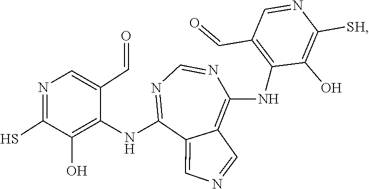 [SMILES: O=Cc1cnc(S)c(O)c1Nc1ncnc(Nc2c(C=O)cnc(S)c2O)c2cncc1-2] | V-397 |
| 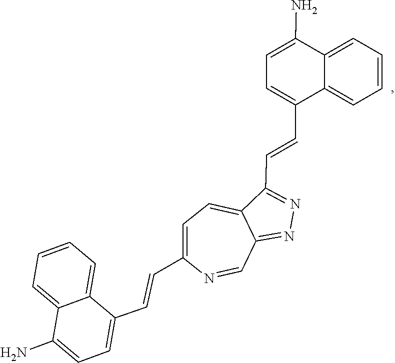 [SMILES: Nc1ccc(/C=C/c2ccc3c(/C=C/c4ccc(N)c5ccccc45)nnc-3cn2)c2ccccc12] | V-398 |
| 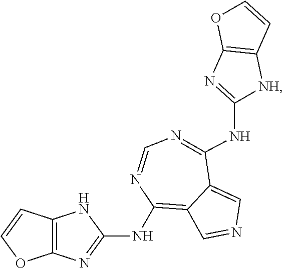 [SMILES: c1nc(Nc2nc3occc3[nH]2)c2cncc-2c(Nc2nc3occc3[nH]2)n1] | V-399 |
| 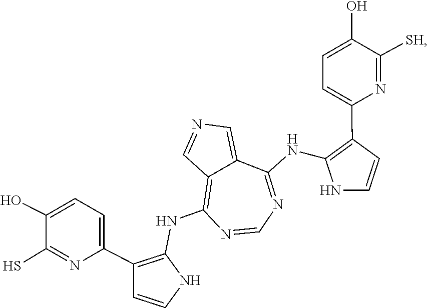 [SMILES: Oc1ccc(-c2cc[nH]c2Nc2ncnc(Nc3[nH]ccc3-c3ccc(O)c(S)n3)c3cncc2-3)nc1S] | V-400 |
| 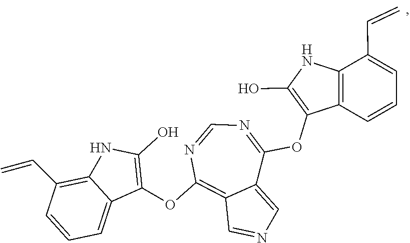 [SMILES: C=Cc1cccc2c(Oc3ncnc(Oc4c(O)[nH]c5c(C=C)cccc45)c4cncc3-4)c(O)[nH]c12] | V-401 |
| 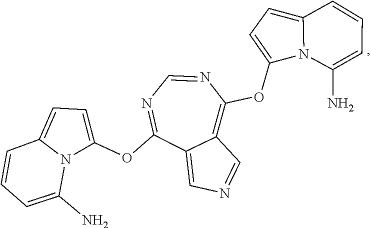 [SMILES: Nc1cccc2ccc(Oc3ncnc(Oc4ccc5cccc(N)n45)c4cncc3-4)n12] | V-402 |
| 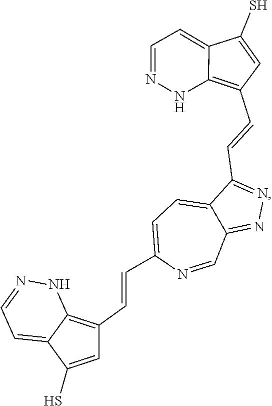 [SMILES: Sc1cc(/C=C/c2ccc3c(/C=C/c4cc(S)c5ccn[nH]c4-5)nnc-3cn2)c2[nH]nccc1-2] | V-403 |
| 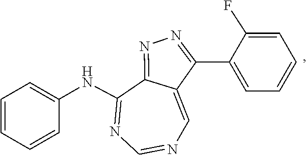 [SMILES: Fc1ccccc1-c1nnc2c(Nc3ccccc3)ncncc1-2] | V-404 |
| 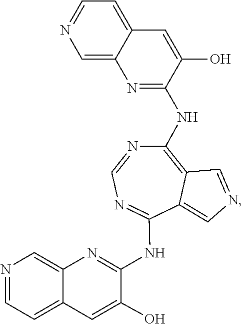 [SMILES: Oc1cc2ccncc2nc1Nc1ncnc(Nc2nc3cnccc3cc2O)c2cncc1-2] | V-405 |
| 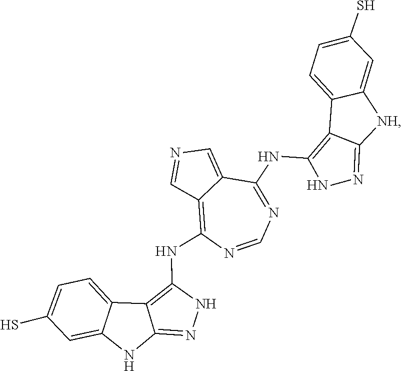 [SMILES: Sc1ccc2c(c1)[nH]c1n[nH]c(Nc3ncnc(Nc4[nH]nc5[nH]c6cc(S)ccc6c45)c4cncc3-4)c12] | V-406 |
| 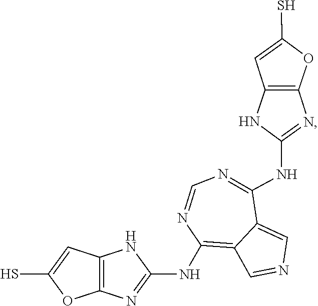 [SMILES: Sc1cc2[nH]c(Nc3ncnc(Nc4nc5oc(S)cc5[nH]4)c4cncc3-4)nc2o1] | V-407 |
| 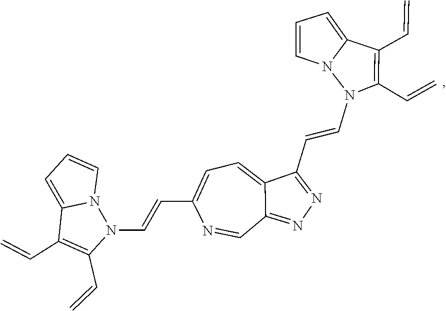 | V-408 |
| 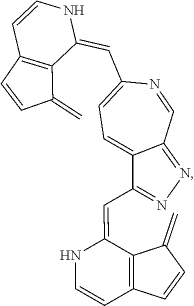 | V-409 |
| 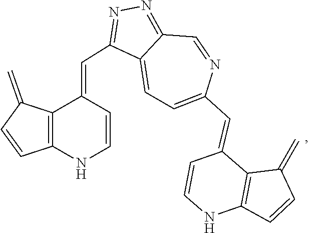 [SMILES: C=c1ccc2[nH]cc/c(=C\c3ccc4c(/C=c5\cc[nH]c6ccc(=C)c5=6)nnc-4cn3)c1=2] | V-410 |
| 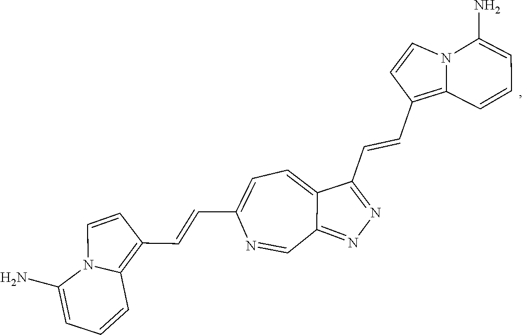 | V-411 |
| 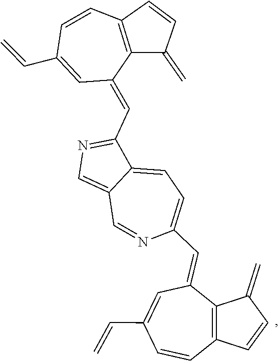 [SMILES: C=Cc1ccc2ccc(=C)c=2/c(=C/c2ccc3c(/C=c4\cc(C=C)ccc5ccc(=C)c4=5)ncc-3cn2)c1] | V-412 |
| 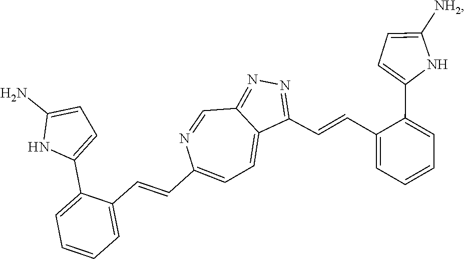 [SMILES: Nc1ccc(-c2ccccc2/C=C/c2ccc3c(/C=C/c4ccccc4-c4ccc(N)[nH]4)nnc-3cn2)[nH]1] | V-413 |
| 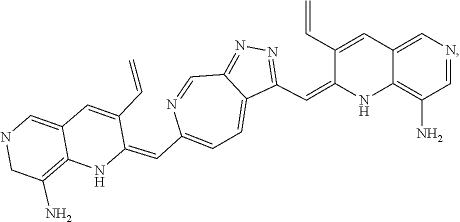 | V-414 |
| 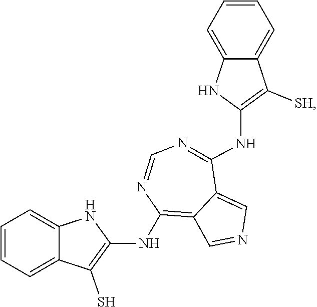 [SMILES: Sc1c(Nc2ncnc(Nc3[nH]c4ccccc4c3S)c3cncc2-3)[nH]c2ccccc12] | V-415 |
| 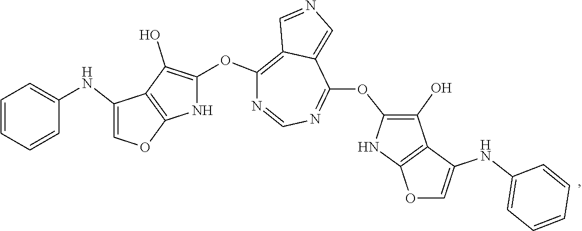 [SMILES: Oc1c(Oc2ncnc(Oc3[nH]c4occ(Nc5ccccc5)c4c3O)c3cncc2-3)[nH]c2occ(Nc3ccccc3)c12] | V-416 |
| 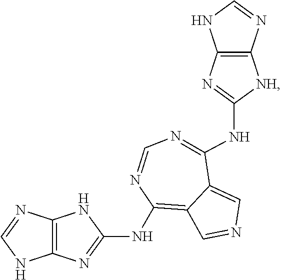 [SMILES: c1nc(Nc2nc3[nH]cnc3[nH]2)c2cncc-2c(Nc2nc3[nH]cnc3[nH]2)n1] | V-417 |
| 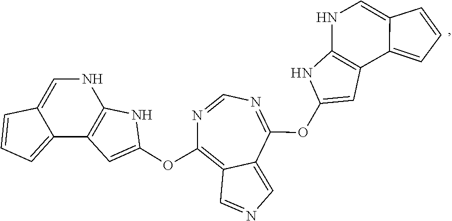 [SMILES: c1cc2c[nH]c3[nH]c(Oc4ncnc(Oc5cc6c7cccc-7c[nH]c6[nH]5)c5cncc4-5)cc3c-2c1] | V-418 |
| 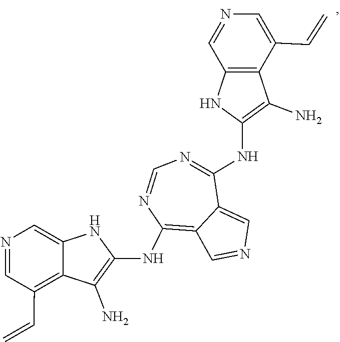 [SMILES: C=Cc1cncc2[nH]c(Nc3ncnc(Nc4[nH]c5cncc(C=C)c5c4N)c4cncc3-4)c(N)c12] | V-419 |
| 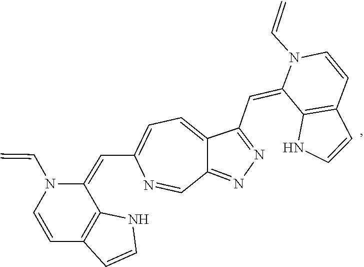 | V-420 |
| 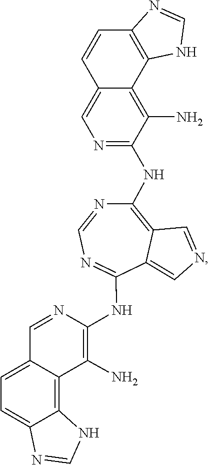 [SMILES: Nc1c(Nc2ncnc(Nc3ncc4ccc5nc[nH]c5c4c3N)c3cncc2-3)ncc2ccc3nc[nH]c3c12] | V-421 |
| 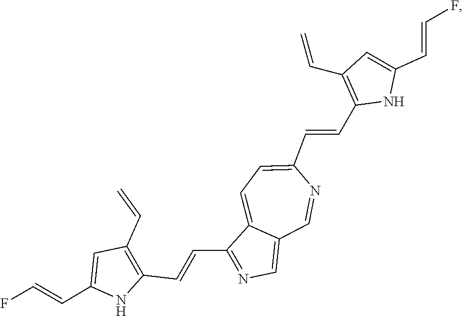 [SMILES: C=Cc1cc(/C=C/F)[nH]c1/C=C/c1ccc2c(/C=C/c3[nH]c(/C=C/F)cc3C=C)ncc-2cn1] | V-422 |
| 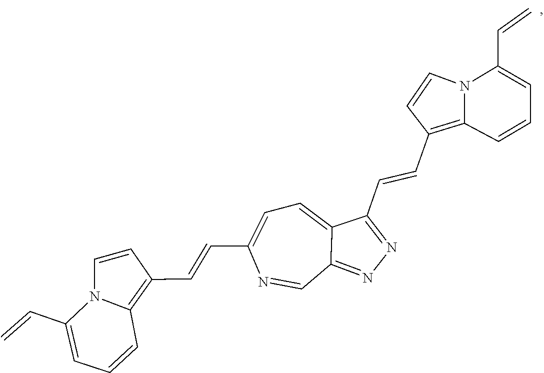 | V-423 |
| 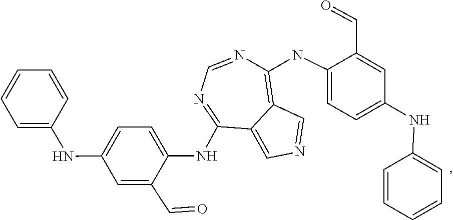 | V-424 |
| 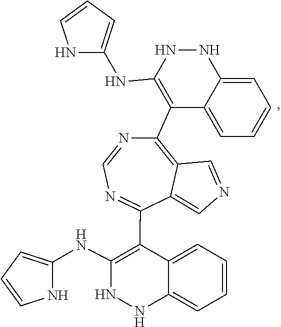 [SMILES: c1c[nH]c(NC2=C(c3ncnc(C4=C(Nc5ccc[nH]5)NNc5ccccc54)c4cncc3-4)c3ccccc3NN2)c1] | V-425 |
| 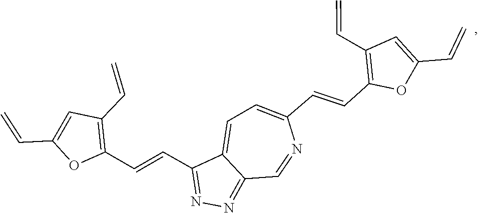 [SMILES: C=Cc1cc(C=C)c(/C=C/c2ccc3c(/C=C/c4oc(C=C)cc4C=C)nnc-3cn2)o1] | V-426 |
| 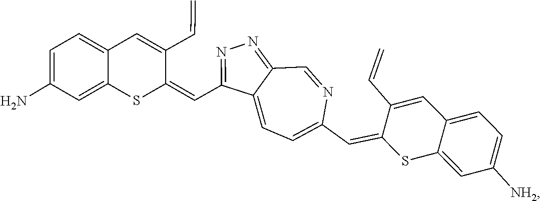 [SMILES: C=CC1=Cc2ccc(N)cc2S/C1=C/c1ccc2c(/C=C3/Sc4cc(N)ccc4C=C3C=C)nnc-2cn1] | V-427 |
| 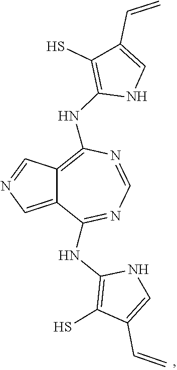 [SMILES: C=Cc1c[nH]c(Nc2ncnc(Nc3[nH]cc(C=C)c3S)c3cncc2-3)c1S] | V-428 |
| 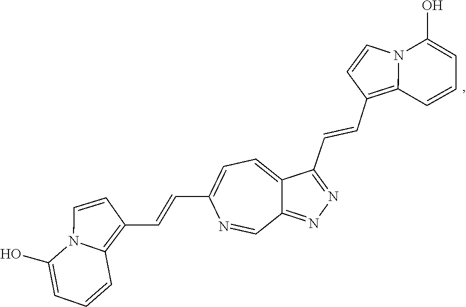 [SMILES: Oc1cccc2c(/C=C/c3ccc4c(/C=C/c5ccn6c(O)cccc56)nnc-4cn3)ccn12] | V-429 |
| 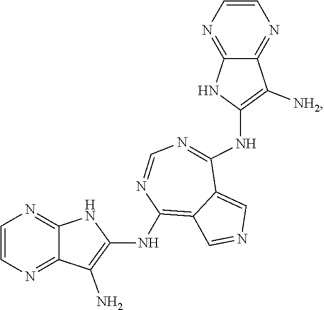 [SMILES: Nc1c(Nc2ncnc(Nc3[nH]c4nccnc4c3N)c3cncc2-3)[nH]c2nccnc12] | V-430 |
| 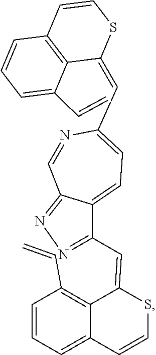 | V-431 |
| 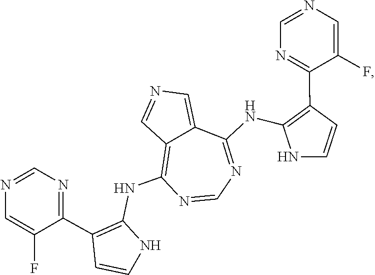 [SMILES: Fc1cncnc1-c1cc[nH]c1Nc1ncnc(Nc2[nH]ccc2-c2ncncc2F)c2cncc1-2] | V-432 |
| 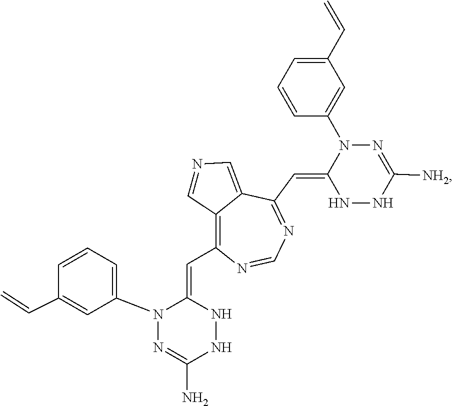 [SMILES: C=Cc1cccc(N2N=C(N)NN/C2=C\c2ncnc(/C=C3\NNC(N)=NN3c3cccc(C=C)c3)c3cncc2-3)c1] | V-433 |
| 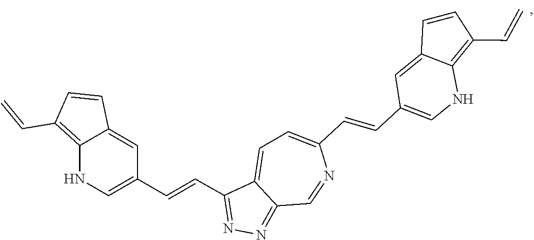 [SMILES: C=Cc1ccc2cc(/C=C/c3ccc4c(/C=C/c5c[nH]c6c(C=C)ccc-6c5)nnc-4cn3)c[nH]c1-2] | V-434 |
| 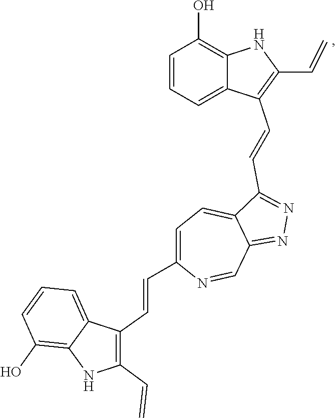 [SMILES: C=Cc1[nH]c2c(O)cccc2c1/C=C/c1ccc2c(/C=C/c3c(C=C)[nH]c4c(O)cccc34)nnc-2cn1] | V-435 |
| 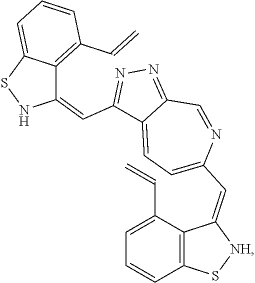 [SMILES: C=Cc1cccc2c1/C(=C\c1ccc3c(/C=C4/NSc5cccc(C=C)c54)nnc-3cn1)NS2] | V-436 |
| 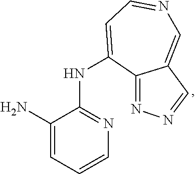 [SMILES: Nc1cccnc1Nc1ccncc2cnnc1-2] | V-437 |
| 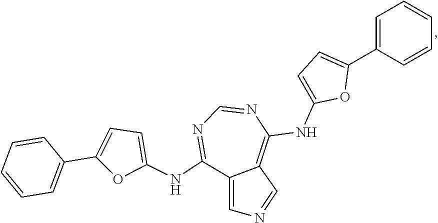 [SMILES: c1ccc(-c2ccc(Nc3ncnc(Nc4ccc(-c5ccccc5)o4)c4cncc3-4)o2)cc1] | V-438 |
| 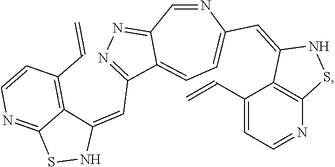 [SMILES: C=Cc1ccnc2c1/C(=C\c1ccc3c(/C=C4/NSc5nccc(C=C)c54)nnc-3cn1)NS2] | V-439 |
| 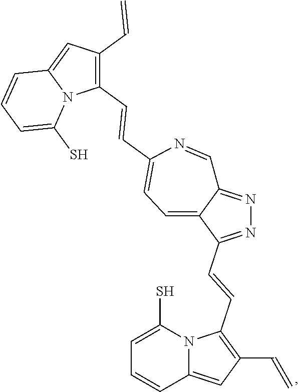 [SMILES: C=Cc1cc2cccc(S)n2c1/C=C/c1ccc2c(/C=C/c3c(C=C)cc4cccc(S)n34)nnc-2cn1] | V-440 |
| 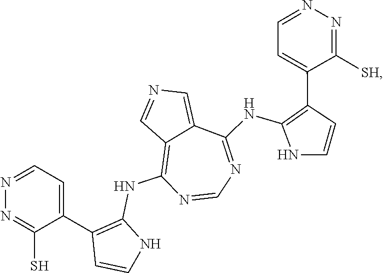 [SMILES: Sc1nnccc1-c1cc[nH]c1Nc1ncnc(Nc2[nH]ccc2-c2ccnnc2S)c2cncc1-2] | V-441 |
| 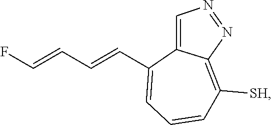 [SMILES: F/C=C/C=C/c1cccc(S)c2nncc1-2] | V-442 |
| 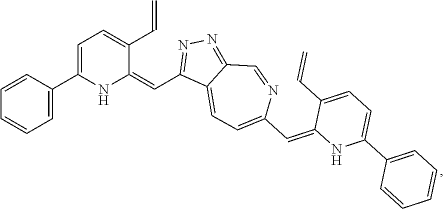 [SMILES: C=CC1=CC=C(c2ccccc2)N/C1=C/c1ccc2c(/C=C3/NC(c4ccccc4)=CC=C3C=C)nnc-2cn1] | V-443 |
| 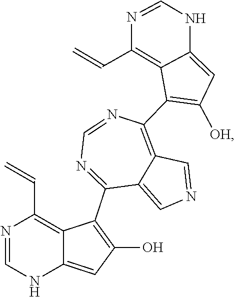 [SMILES: C=Cc1nc[nH]c2cc(O)c(-c3ncnc(-c4c(O)cc5[nH]cnc(C=C)c4-5)c4cncc3-4)c1-2] | V-444 |
| 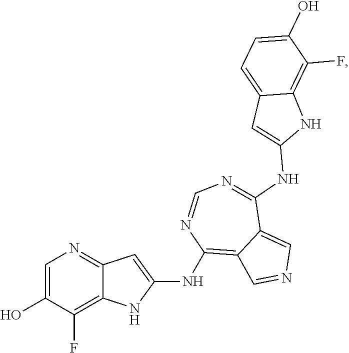 [SMILES: Oc1ccc2cc(Nc3ncnc(Nc4cc5ncc(O)c(F)c5[nH]4)c4cncc3-4)[nH]c2c1F] | V-445 |
| 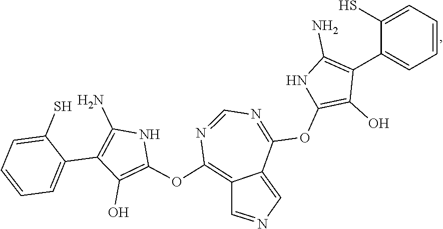 [SMILES: Nc1[nH]c(Oc2ncnc(Oc3[nH]c(N)c(-c4ccccc4S)c3O)c3cncc2-3)c(O)c1-c1ccccc1S] | V-446 |
| 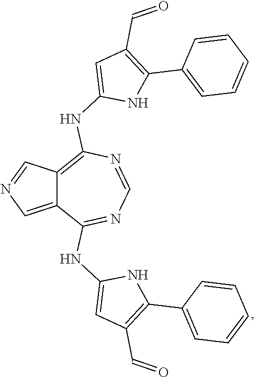 [SMILES: O=Cc1cc(Nc2ncnc(Nc3cc(C=O)c(-c4ccccc4)[nH]3)c3cncc2-3)[nH]c1-c1ccccc1] | V-447 |
| 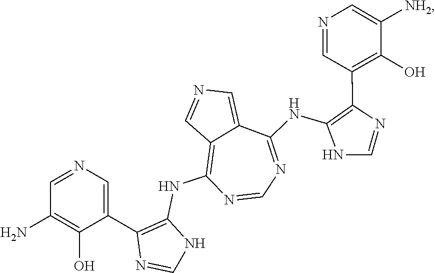 [SMILES: Nc1cncc(-c2nc[nH]c2Nc2ncnc(Nc3[nH]cnc3-c3cncc(N)c3O)c3cncc2-3)c1O] | V-448 |
| 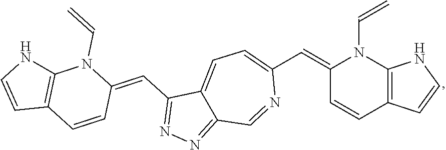 [SMILES: C=CN1/C(=C/c2ccc3c(/C=C4\C=Cc5cc[nH]c5N4C=C)nnc-3cn2)C=Cc2cc[nH]c21] | V-449 |
| 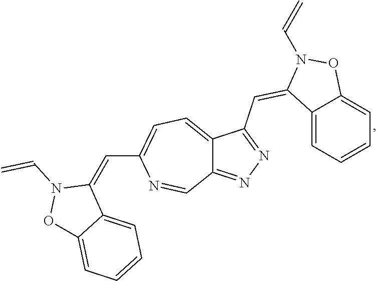 [SMILES: C=CN1Oc2ccccc2/C1=C\c1ccc2c(/C=C3\c4ccccc4ON3C=C)nnc-2cn1] | V-450 |
| 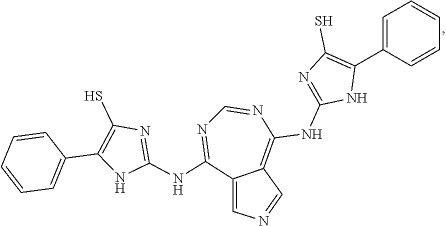 [SMILES: Sc1nc(Nc2ncnc(Nc3nc(S)c(-c4ccccc4)[nH]3)c3cncc2-3)[nH]c1-c1ccccc1] | V-451 |
| 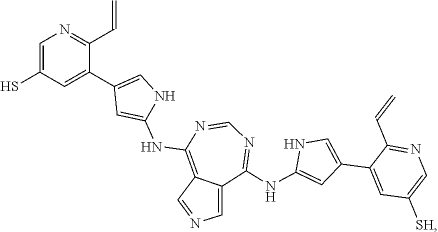 [SMILES: C=Cc1ncc(S)cc1-c1c[nH]c(Nc2ncnc(Nc3cc(-c4cc(S)cnc4C=C)c[nH]3)c3cncc2-3)c1] | V-452 |
| 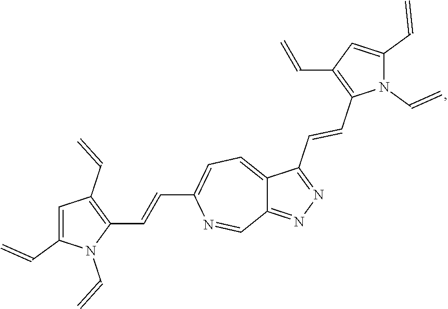 [SMILES: C=Cc1cc(C=C)n(C=C)c1/C=C/c1ccc2c(/C=C/c3c(C=C)cc(C=C)n3C=C)nnc-2cn1] | V-453 |
| 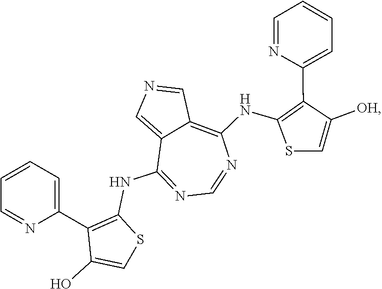 [SMILES: Oc1csc(Nc2ncnc(Nc3scc(O)c3-c3ccccn3)c3cncc2-3)c1-c1ccccn1] | V-454 |
| 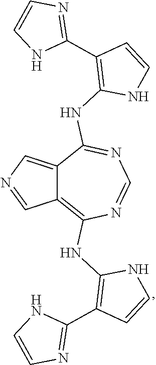 [SMILES: c1c[nH]c(-c2cc[nH]c2Nc2ncnc(Nc3[nH]ccc3-c3ncc[nH]3)c3cncc2-3)n1] | V-455 |
| 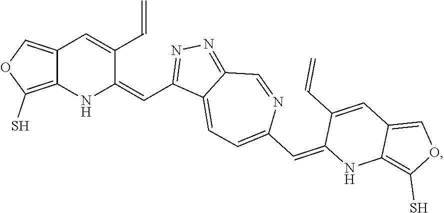 [SMILES: C=CC1=Cc2coc(S)c2N/C1=C/c1ccc2c(/C=C3/Nc4c(coc4S)C=C3C=C)nnc-2cn1] | V-456 |
| 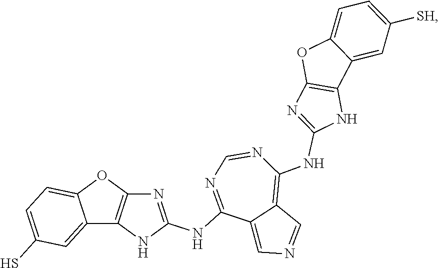 [SMILES: Sc1ccc2oc3nc(Nc4ncnc(Nc5nc6oc7ccc(S)cc7c6[nH]5)c5cncc4-5)[nH]c3c2c1] | V-457 |
| 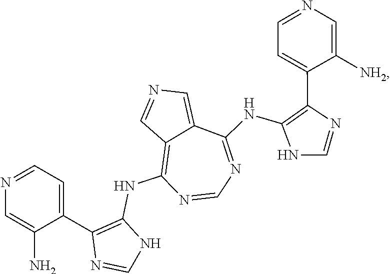 [SMILES: Nc1cnccc1-c1nc[nH]c1Nc1ncnc(Nc2[nH]cnc2-c2ccncc2N)c2cncc1-2] | V-458 |
| 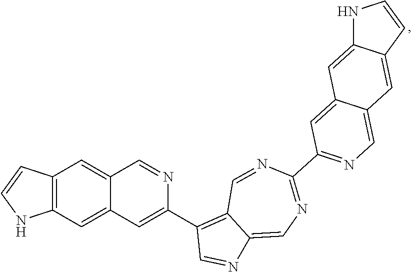 [SMILES: c1cc2cc3cnc(-c4ncc5ncc(-c6cc7cc8[nH]ccc8cc7cn6)c-5cn4)cc3cc2[nH]1] | V-459 |
| 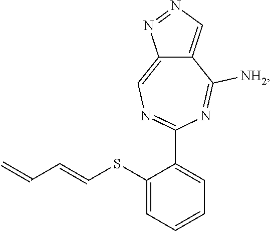 [SMILES: C=C/C=C/Sc1ccccc1-c1ncc2nncc-2c(N)n1] | V-460 |
| 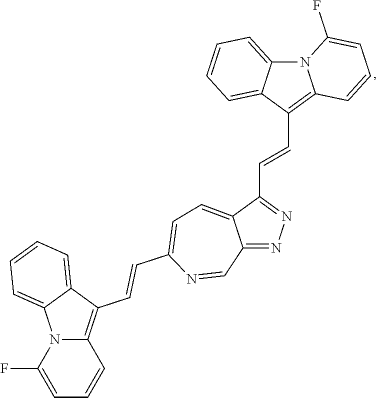 [SMILES: Fc1cccc2c(/C=C/c3ccc4c(/C=C/c5c6ccccc6n6c(F)cccc56)nnc-4cn3)c3ccccc3n12] | V-461 |
| 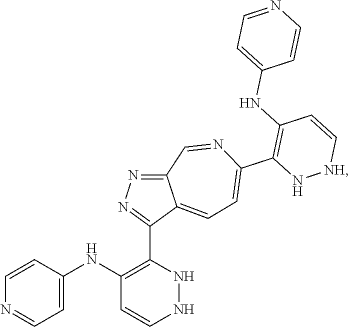 [SMILES: C1=CC(Nc2ccncc2)=C(c2ccc3c(C4=C(Nc5ccncc5)C=CNN4)nnc-3cn2)NN1] | V-462 |
| 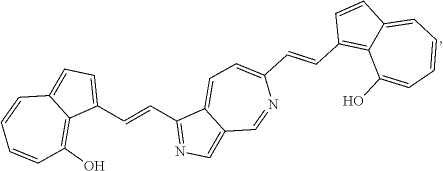 [SMILES: Oc1ccccc2ccc(/C=C/c3ccc4c(/C=C/c5ccc6ccccc(O)c5-6)ncc-4cn3)c1-2] | V-463 |
| 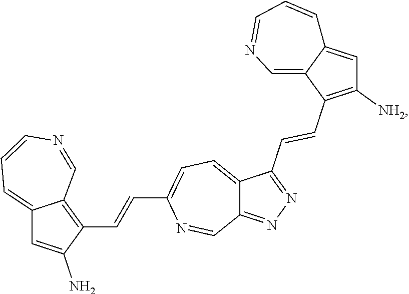 [SMILES: Nc1cc2cccncc-2c1/C=C/c1ccc2c(/C=C/c3c(N)cc4cccncc3-4)nnc-2cn1] | V-464 |
| 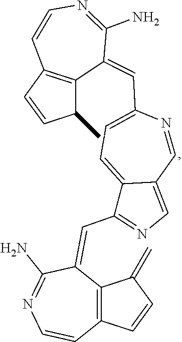 [SMILES: C=c1ccc2ccnc(N)/c(=C/c3ncc4cnc(/C=C5/C(N)=NC=CC6=C5[C@@H](C)C=C6)ccc3-4)c1=2] | V-465 |
| 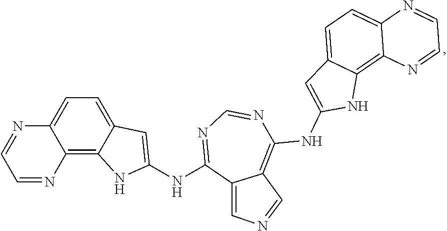 [SMILES: c1nc(Nc2cc3ccc4nccnc4c3[nH]2)c2cncc-2c(Nc2cc3ccc4nccnc4c3[nH]2)n1] | V-466 |
| 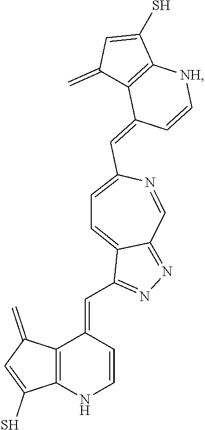 [SMILES: C=c1cc(S)c2[nH]cc/c(=C\c3ccc4c(/C=c5\cc[nH]c6c(S)cc(=C)c5=6)nnc-4cn3)c1=2] | V-467 |
| 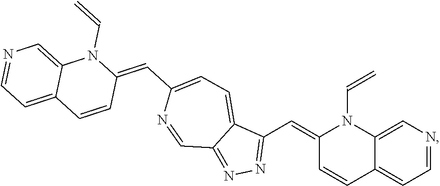 [SMILES: C=CN1/C(=C/c2ccc3c(/C=C4\C=Cc5ccncc5N4C=C)nnc-3cn2)C=Cc2ccncc21] | V-468 |
| 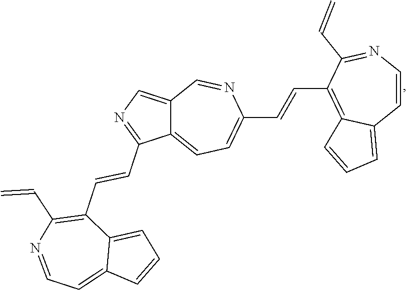 [SMILES: C=Cc1nccc2cccc-2c1/C=C/c1ccc2c(/C=C/c3c4cccc-4ccnc3C=C)ncc-2cn1] | V-469 |
| 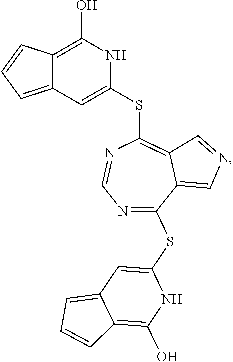 [SMILES: Oc1[nH]c(Sc2ncnc(Sc3cc4cccc-4c(O)[nH]3)c3cncc2-3)cc2cccc1-2] | V-470 |
| 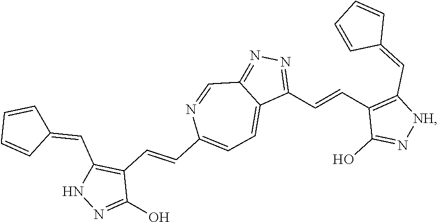 [SMILES: Oc1n[nH]c(C=C2C=CC=C2)c1/C=C/c1ccc2c(/C=C/c3c(O)n[nH]c3C=C3C=CC=C3)nnc-2cn1] | V-471 |
| 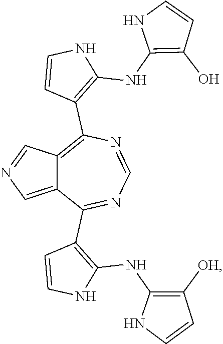 [SMILES: Oc1cc[nH]c1Nc1[nH]ccc1-c1ncnc(-c2cc[nH]c2Nc2[nH]ccc2O)c2cncc1-2] | V-472 |
| 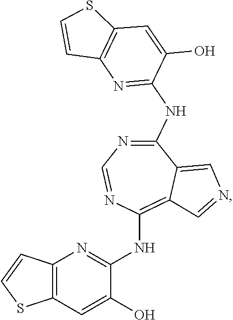 [SMILES: Oc1cc2sccc2nc1Nc1ncnc(Nc2nc3ccsc3cc2O)c2cncc1-2] | V-473 |
| 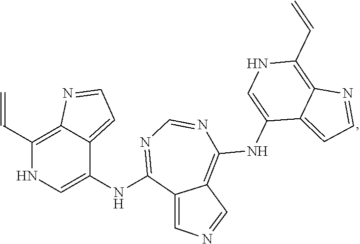 [SMILES: C=Cc1[nH]cc(Nc2ncnc(Nc3c[nH]c(C=C)c4nccc3-4)c3cncc2-3)c2ccnc1-2] | V-474 |
| 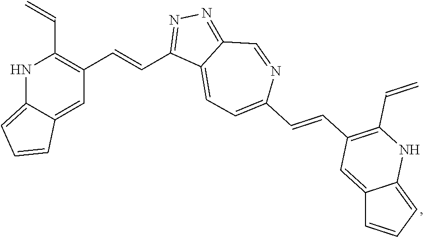 [SMILES: C=Cc1[nH]c2cccc-2cc1/C=C/c1ccc2c(/C=C/c3cc4cccc-4[nH]c3C=C)nnc-2cn1] | V-475 |
| 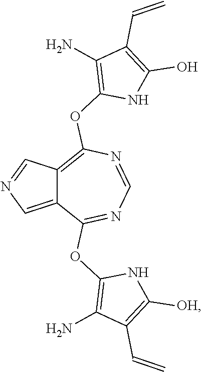 [SMILES: C=Cc1c(O)[nH]c(Oc2ncnc(Oc3[nH]c(O)c(C=C)c3N)c3cncc2-3)c1N] | V-476 |
| 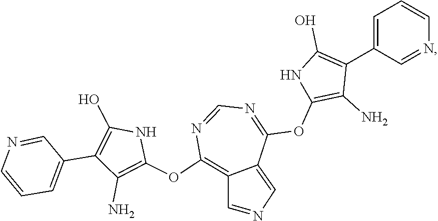 [SMILES: Nc1c(Oc2ncnc(Oc3[nH]c(O)c(-c4cccnc4)c3N)c3cncc2-3)[nH]c(O)c1-c1cccnc1] | V-477 |
| 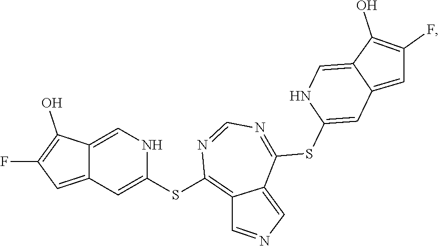 [SMILES: Oc1c(F)cc2cc(Sc3ncnc(Sc4cc5cc(F)c(O)c-5c[nH]4)c4cncc3-4)[nH]cc1-2] | V-478 |
| 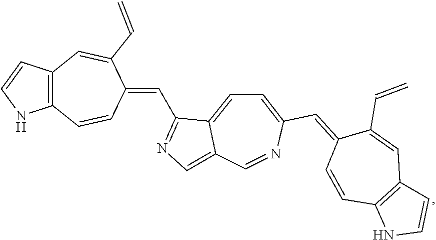 [SMILES: C=CC1=Cc2cc[nH]c2C=C/C1=C\c1ccc2c(/C=C3\C=Cc4[nH]ccc4C=C3C=C)ncc-2cn1] | V-479 |
| 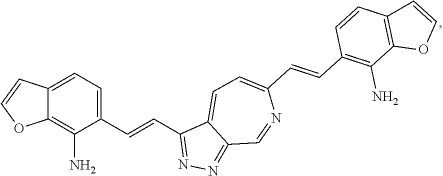 [SMILES: Nc1c(/C=C/c2ccc3c(/C=C/c4ccc5ccoc5c4N)nnc-3cn2)ccc2ccoc12] | V-480 |
| 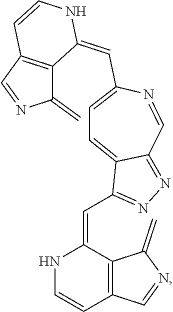 [SMILES: C=c1ncc2cc[nH]/c(=C/c3ccc4c(/C=c5/[nH]ccc6cnc(=C)c5=6)nnc-4cn3)c1=2] | V-481 |
| 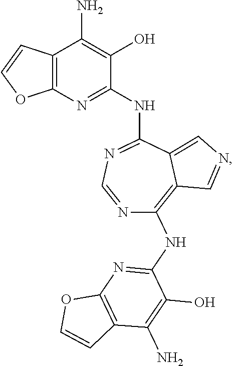 [SMILES: Nc1c(O)c(Nc2ncnc(Nc3nc4occc4c(N)c3O)c3cncc2-3)nc2occc12] | V-482 |
| 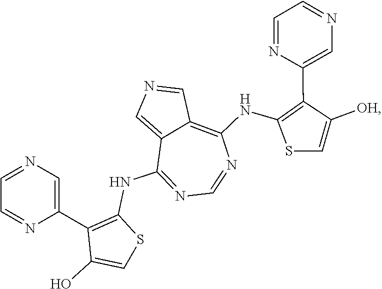 [SMILES: Oc1csc(Nc2ncnc(Nc3scc(O)c3-c3cnccn3)c3cncc2-3)c1-c1cnccn1] | V-483 |
| 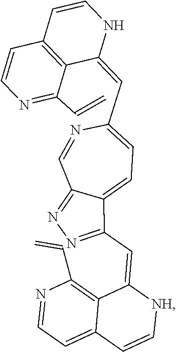 [SMILES: C=Cc1nccc2c1/C(=C\c1ccc3c(/C=C4/NC=Cc5ccnc(C=C)c54)nnc-3cn1)NC=C2] | V-484 |
| 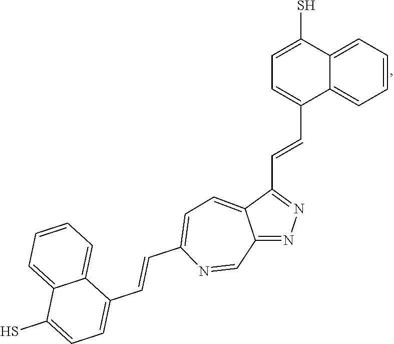 [SMILES: Sc1ccc(/C=C/c2ccc3c(/C=C/c4ccc(S)c5ccccc45)nnc-3cn2)c2ccccc12] | V-485 |
| 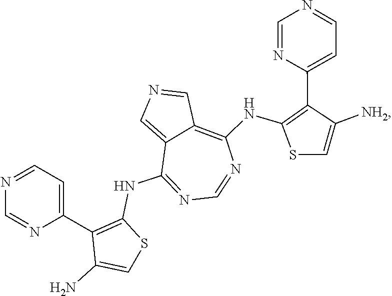 [SMILES: Nc1csc(Nc2ncnc(Nc3scc(N)c3-c3ccncn3)c3cncc2-3)c1-c1ccncn1] | V-486 |
| 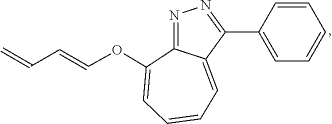 [SMILES: C=C/C=C/Oc1ccccc2c(-c3ccccc3)nnc1-2] | V-487 |
| 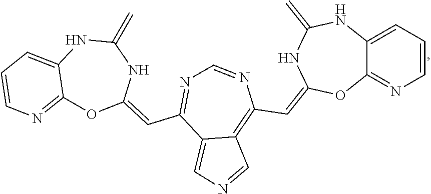 [SMILES: C=c1[nH]/c(=C\c2ncnc(/C=c3\[nH]c(=C)[nH]c4cccnc4o3)c3cncc2-3)oc2ncccc2[nH]1] | V-488 |
| 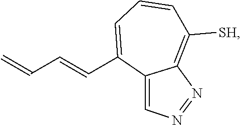 | V-489 |
| 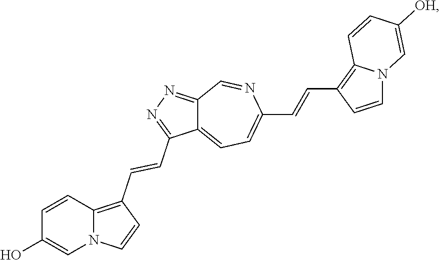 [SMILES: Oc1ccc2c(/C=C/c3ccc4c(/C=C/c5ccn6cc(O)ccc56)nnc-4cn3)ccn2c1] | V-490 |
| 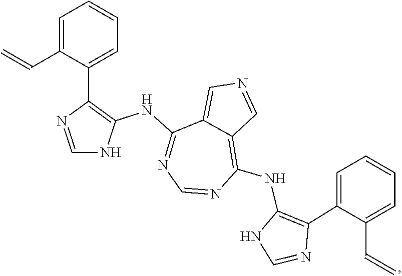 [SMILES: C=Cc1ccccc1-c1nc[nH]c1Nc1ncnc(Nc2[nH]cnc2-c2ccccc2C=C)c2cncc1-2] | V-491 |
| 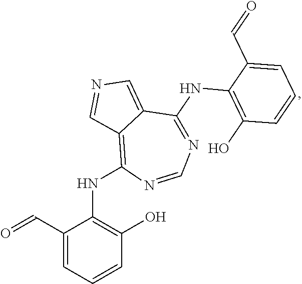 [SMILES: O=Cc1cccc(O)c1Nc1ncnc(Nc2c(O)cccc2C=O)c2cncc1-2] | V-492 |
| 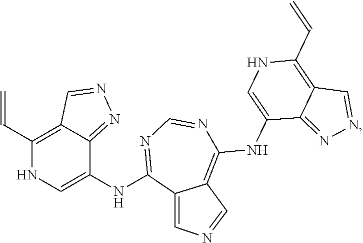 [SMILES: C=Cc1[nH]cc(Nc2ncnc(Nc3c[nH]c(C=C)c4cnnc3-4)c3cncc2-3)c2nncc1-2] | V-493 |
| 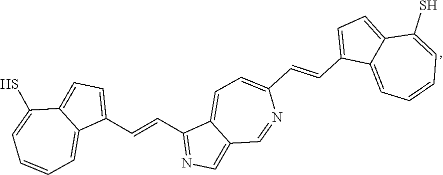 [SMILES: Sc1ccccc2c(/C=C/c3ccc4c(/C=C/c5ccc6c(S)ccccc5-6)ncc-4cn3)ccc1-2] | V-494 |
| 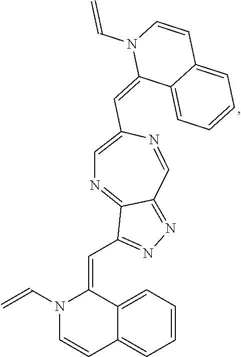 [SMILES: C=CN1C=Cc2ccccc2/C1=C\c1cnc2c(/C=C3\c4ccccc4C=CN3C=C)nnc-2cn1] | V-495 |
| 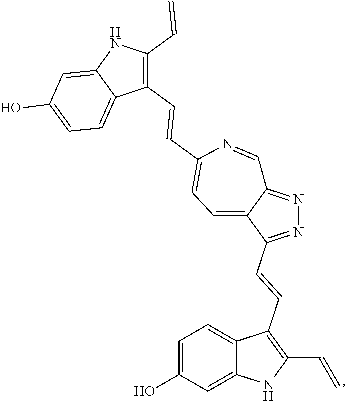 [SMILES: C=Cc1[nH]c2cc(O)ccc2c1/C=C/c1ccc2c(/C=C/c3c(C=C)[nH]c4cc(O)ccc34)nnc-2cn1] | V-496 |
| 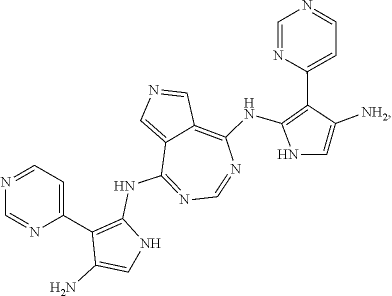 [SMILES: Nc1c[nH]c(Nc2ncnc(Nc3[nH]cc(N)c3-c3ccncn3)c3cncc2-3)c1-c1ccncn1] | V-497 |
| 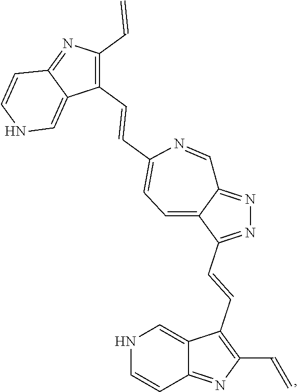 [SMILES: C=Cc1nc2cc[nH]cc-2c1/C=C/c1ccc2c(/C=C/c3c4c[nH]ccc-4nc3C=C)nnc-2cn1] | V-498 |
| 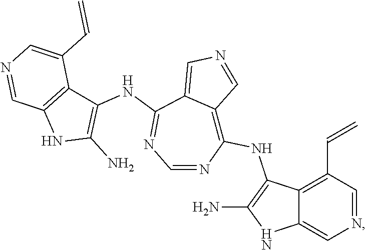 [SMILES: C=Cc1cncc2[nH]c(N)c(Nc3ncnc(Nc4c(N)[nH]c5cncc(C=C)c45)c4cncc3-4)c12] | V-499 |
| 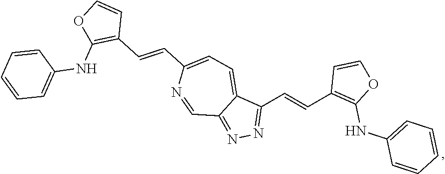 [SMILES: C(=C\c1ccoc1Nc1ccccc1)/c1ccc2c(/C=C/c3ccoc3Nc3ccccc3)nnc-2cn1] | V-500 |
| 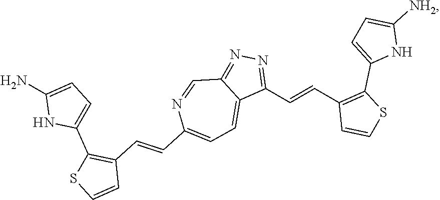 [SMILES: Nc1ccc(-c2sccc2/C=C/c2ccc3c(/C=C/c4ccsc4-c4ccc(N)[nH]4)nnc-3cn2)[nH]1] | V-501 |
| 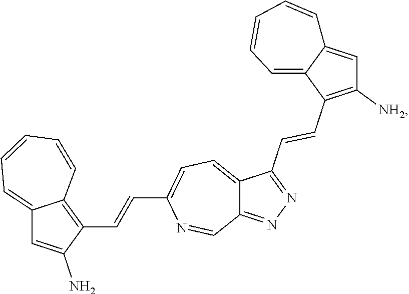 [SMILES: Nc1cc2cccccc-2c1/C=C/c1ccc2c(/C=C/c3c(N)cc4cccccc3-4)nnc-2cn1] | V-502 |
| 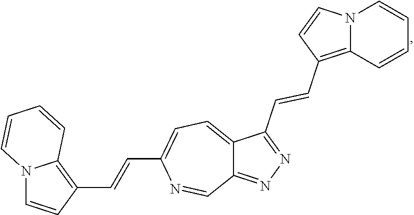 [SMILES: C(=C\c1ccn2ccccc12)/c1ccc2c(/C=C/c3ccn4ccccc34)nnc-2cn1] | V-503 |
| 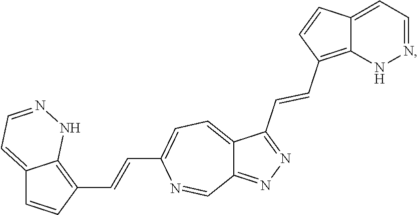 [SMILES: C(=C\c1ccc2ccn[nH]c1-2)/c1ccc2c(/C=C/c3ccc4ccn[nH]c3-4)nnc-2cn1] | V-504 |
| 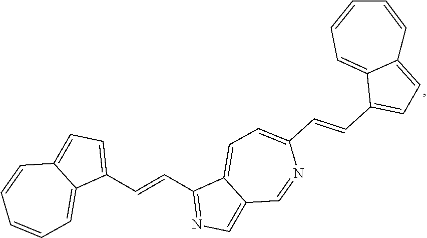 [SMILES: C(=C\c1ccc2cccccc1-2)/c1ccc2c(/C=C/c3ccc4cccccc3-4)ncc-2cn1] | V-505 |
| 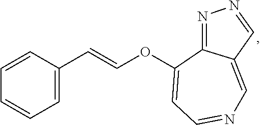 [SMILES: C(=C\c1ccccc1)/Oc1ccncc2cnnc1-2] | V-506 |
| 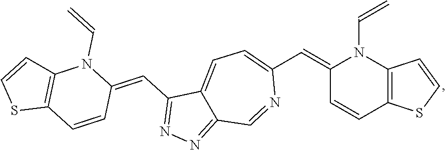 [SMILES: C=CN1/C(=C/c2ccc3c(/C=C4\C=Cc5sccc5N4C=C)nnc-3cn2)C=Cc2sccc21] | V-507 |
| 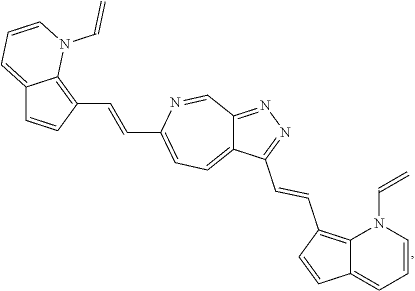 [SMILES: C=Cn1cccc2ccc(/C=C/c3ccc4c(/C=C/c5ccc6cccn(C=C)c5-6)nnc-4cn3)c1-2] | V-508 |
| 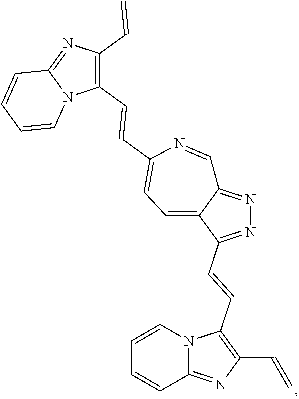 [SMILES: C=Cc1nc2ccccn2c1/C=C/c1ccc2c(/C=C/c3c(C=C)nc4ccccn34)nnc-2cn1] | V-509 |
| 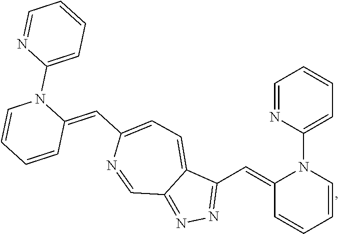 [SMILES: C1=C/C(=C\c2ccc3c(/C=C4\C=CC=CN4c4ccccn4)nnc-3cn2)N(c2ccccn2)C=C1] | V-510 |
| 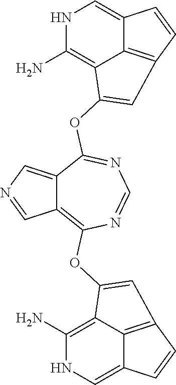 [SMILES: Nc1[nH]cc2ccc3cc(Oc4ncnc(Oc5cc6ccc7c[nH]c(N)c5c76)c5cncc4-5)c1c23] | V-511 |
| 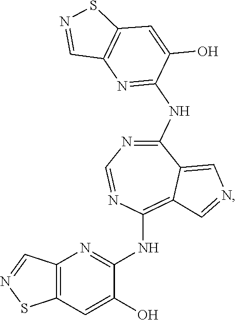 [SMILES: Oc1cc2sncc2nc1Nc1ncnc(Nc2nc3cnsc3cc2O)c2cncc1-2] | V-512 |
| 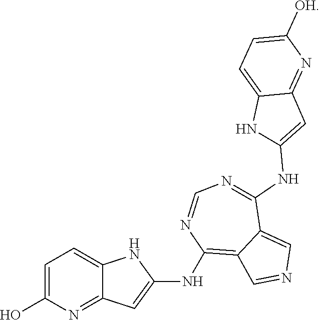 [SMILES: Oc1ccc2[nH]c(Nc3ncnc(Nc4cc5nc(O)ccc5[nH]4)c4cncc3-4)cc2n1] | V-513 |
| 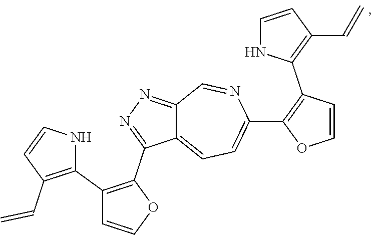 [SMILES: C=Cc1cc[nH]c1-c1ccoc1-c1ccc2c(-c3occc3-c3[nH]ccc3C=C)nnc-2cn1] | V-514 |
| 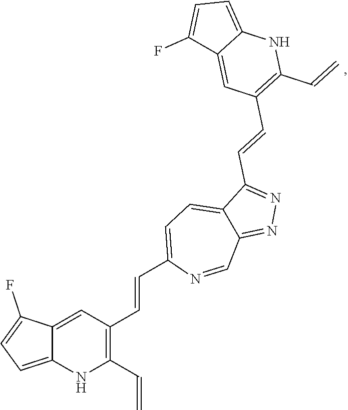 [SMILES: C=Cc1[nH]c2ccc(F)c-2cc1/C=C/c1ccc2c(/C=C/c3cc4c(F)ccc-4[nH]c3C=C)nnc-2cn1] | V-515 |
| 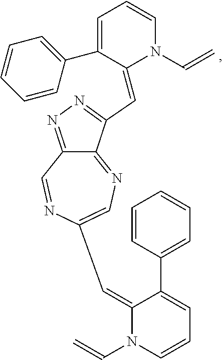 [SMILES: C=CN1C=CC=C(c2ccccc2)/C1=C\c1cnc2c(/C=C3\C(c4ccccc4)=CC=CN3C=C)nnc-2cn1] | V-516 |
| 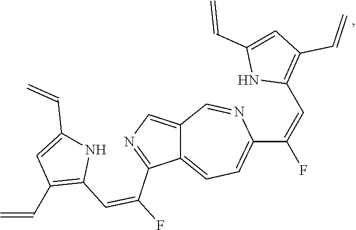 [SMILES: C=Cc1cc(C=C)c(/C=C(/F)c2ccc3c(/C(F)=C\c4[nH]c(C=C)cc4C=C)ncc-3cn2)[nH]1] | V-517 |
| 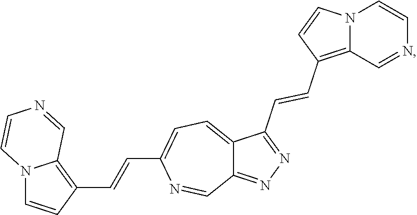 [SMILES: C(=C\c1ccn2ccncc12)/c1ccc2c(/C=C/c3ccn4ccncc34)nnc-2cn1] | V-518 |
| 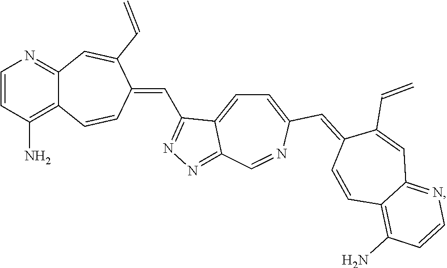 [SMILES: C=CC1=Cc2nccc(N)c2C=C/C1=C\c1ccc2c(/C=C3\C=Cc4c(N)ccnc4C=C3C=C)nnc-2cn1] | V-519 |
| 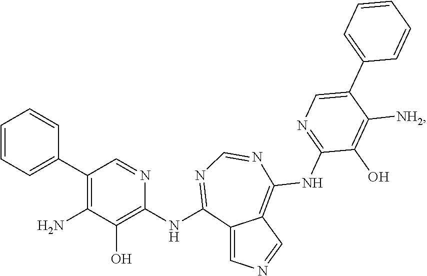 [SMILES: Nc1c(-c2ccccc2)cnc(Nc2ncnc(Nc3ncc(-c4ccccc4)c(N)c3O)c3cncc2-3)c1O] | V-520 |
| 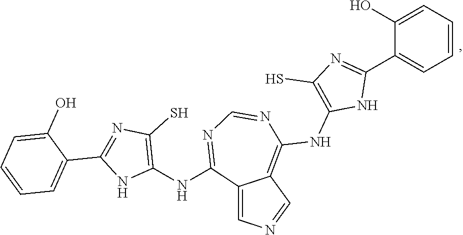 [SMILES: Oc1ccccc1-c1nc(S)c(Nc2ncnc(Nc3[nH]c(-c4ccccc4O)nc3S)c3cncc2-3)[nH]1] | V-521 |
| 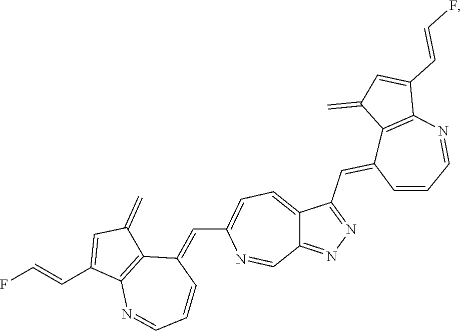 [SMILES: C=c1cc(/C=C/F)c2nccc/c(=C\c3ccc4c(/C=c5\cccnc6c(/C=C/F)cc(=C)c5=6)nnc-4cn3)c1=2] | V-522 |
| 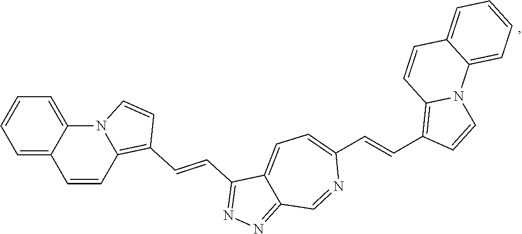 [SMILES: C(=C\c1ccn2c1ccc1ccccc12)/c1ccc2c(/C=C/c3ccn4c3ccc3ccccc34)nnc-2cn1] | V-523 |
| 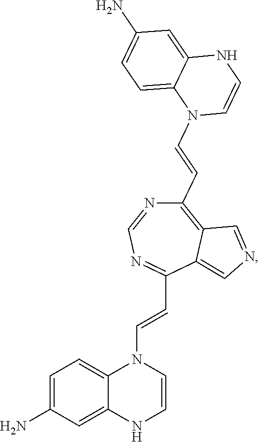 [SMILES: Nc1ccc2c(c1)NC=CN2/C=C/c1ncnc(/C=C/N2C=CNc3cc(N)ccc32)c2cncc1-2] | V-524 |
| 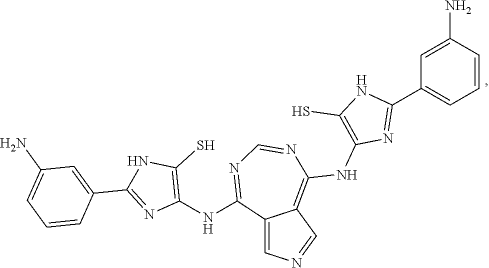 [SMILES: Nc1cccc(-c2nc(Nc3ncnc(Nc4nc(-c5cccc(N)c5)[nH]c4S)c4cncc3-4)c(S)[nH]2)c1] | V-525 |
| 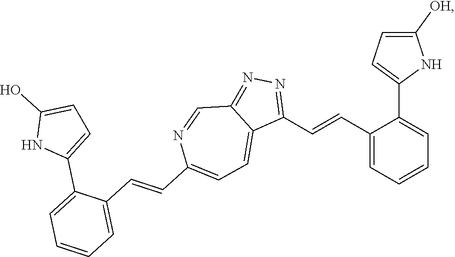 [SMILES: Oc1ccc(-c2ccccc2/C=C/c2ccc3c(/C=C/c4ccccc4-c4ccc(O)[nH]4)nnc-3cn2)[nH]1] | V-526 |
| 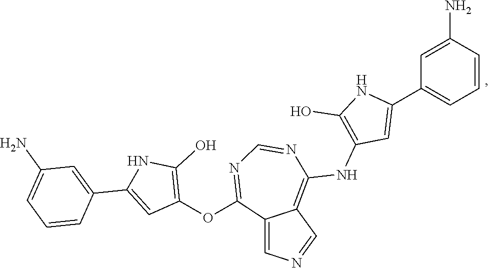 [SMILES: Nc1cccc(-c2cc(Nc3ncnc(Oc4cc(-c5cccc(N)c5)[nH]c4O)c4cncc3-4)c(O)[nH]2)c1] | V-527 |
| 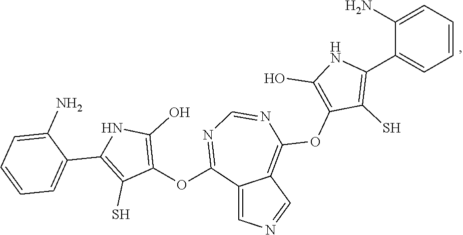 [SMILES: Nc1ccccc1-c1[nH]c(O)c(Oc2ncnc(Oc3c(O)[nH]c(-c4ccccc4N)c3S)c3cncc2-3)c1S] | V-528 |
| 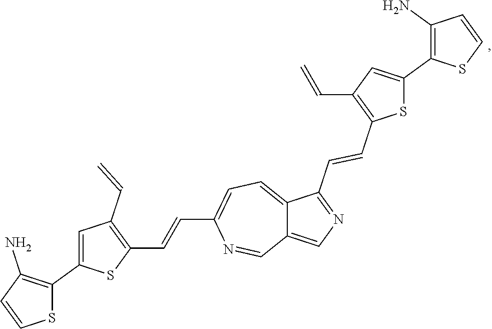 [SMILES: C=Cc1cc(-c2sccc2N)sc1/C=C/c1ccc2c(/C=C/c3sc(-c4sccc4N)cc3C=C)ncc-2cn1] | V-529 |
| 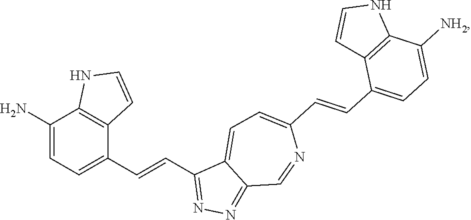 [SMILES: Nc1ccc(/C=C/c2ccc3c(/C=C/c4ccc(N)c5[nH]ccc45)nnc-3cn2)c2cc[nH]c12] | V-530 |
| 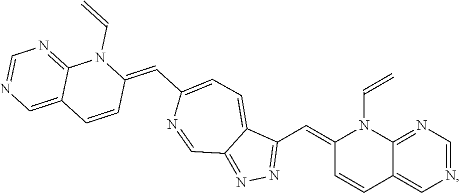 [SMILES: C=CN1/C(=C/c2ccc3c(/C=C4\C=Cc5cncnc5N4C=C)nnc-3cn2)C=Cc2cncnc21] | V-531 |
| 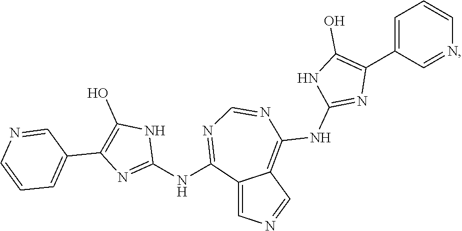 [SMILES: Oc1[nH]c(Nc2ncnc(Nc3nc(-c4cccnc4)c(O)[nH]3)c3cncc2-3)nc1-c1cccnc1] | V-532 |
| 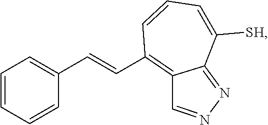 [SMILES: Sc1cccc(/C=C/c2ccccc2)c2cnnc1-2] | V-533 |
| 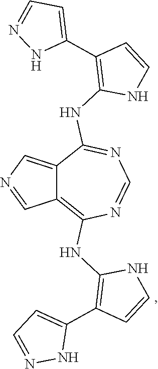 [SMILES: c1cc(-c2cc[nH]c2Nc2ncnc(Nc3[nH]ccc3-c3ccn[nH]3)c3cncc2-3)[nH]n1] | V-534 |
| 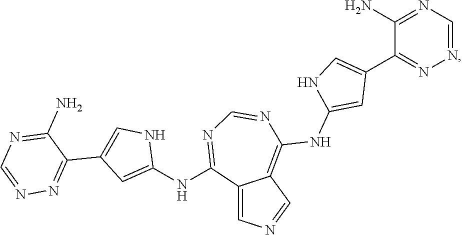 [SMILES: Nc1ncnnc1-c1c[nH]c(Nc2ncnc(Nc3cc(-c4nncnc4N)c[nH]3)c3cncc2-3)c1] | V-535 |
| 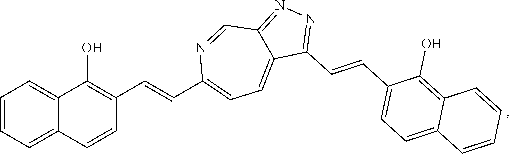 [SMILES: Oc1c(/C=C/c2ccc3c(/C=C/c4ccc5ccccc5c4O)nnc-3cn2)ccc2ccccc12] | V-536 |
| 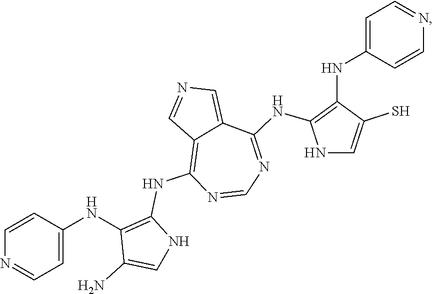 [SMILES: Nc1c[nH]c(Nc2ncnc(Nc3[nH]cc(S)c3Nc3ccncc3)c3cncc2-3)c1Nc1ccncc1] | V-537 |
| 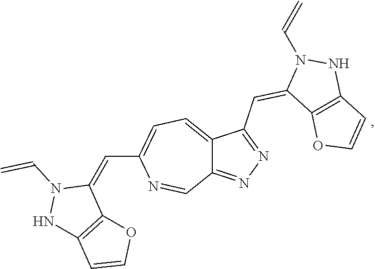 [SMILES: C=CN1Nc2ccoc2/C1=C\c1ccc2c(/C=C3\c4occc4NN3C=C)nnc-2cn1] | V-538 |
| 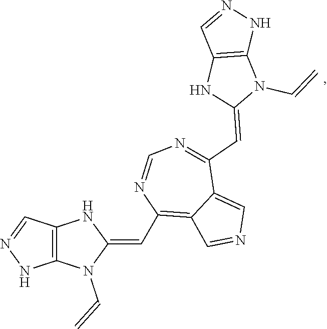 [SMILES: C=CN1/C(=C/c2ncnc(/C=C3\Nc4cn[nH]c4N3C=C)c3cncc2-3)Nc2cn[nH]c21] | V-539 |
| 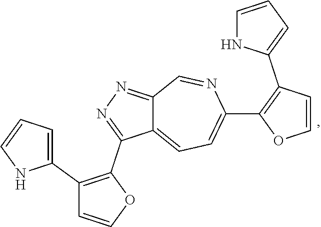 [SMILES: c1c[nH]c(-c2ccoc2-c2ccc3c(-c4occc4-c4ccc[nH]4)nnc-3cn2)c1] | V-540 |
| 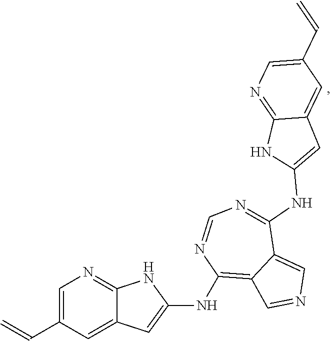 [SMILES: C=Cc1cnc2[nH]c(Nc3ncnc(Nc4cc5cc(C=C)cnc5[nH]4)c4cncc3-4)cc2c1] | V-541 |
| 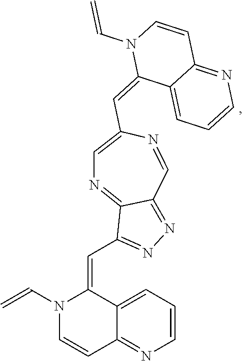 [SMILES: C=CN1C=Cc2ncccc2/C1=C\c1cnc2c(/C=C3\c4cccnc4C=CN3C=C)nnc-2cn1] | V-542 |
| 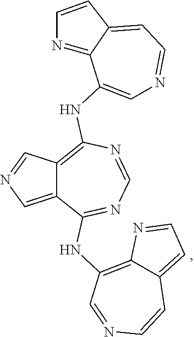 [SMILES: c1cc2ccnc-2c(Nc2ncnc(Nc3cnccc4ccnc3-4)c3cncc2-3)cn1] | V-543 |
| 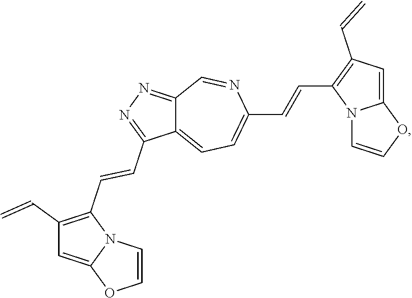 [SMILES: C=Cc1cc2occn2c1/C=C/c1ccc2c(/C=C/c3c(C=C)cc4occn34)nnc-2cn1] | V-544 |
| 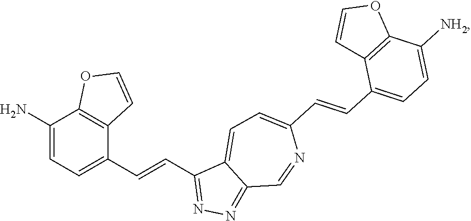 [SMILES: Nc1ccc(/C=C/c2ccc3c(/C=C/c4ccc(N)c5occc45)nnc-3cn2)c2ccoc12] | V-545 |
| 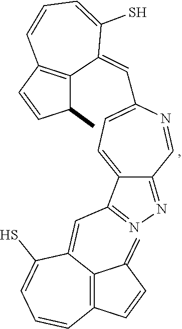 [SMILES: C=c1ccc2cccc(S)/c(=C/c3nnc4cnc(/C=C5/C(S)=CC=CC6=C5[C@@H](C)C=C6)ccc3-4)c1=2] | V-546 |
| 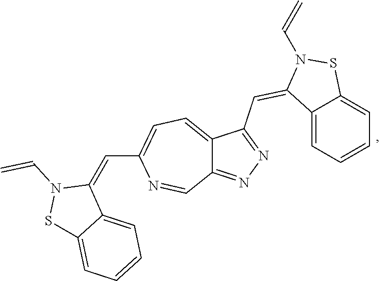 [SMILES: C=CN1Sc2ccccc2/C1=C\c1ccc2c(/C=C3\c4ccccc4SN3C=C)nnc-2cn1] | V-547 |
| 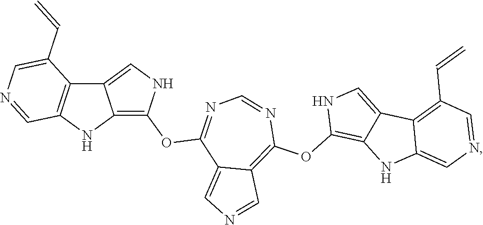 [SMILES: C=Cc1cncc2[nH]c3c(Oc4ncnc(Oc5[nH]cc6c5[nH]c5cncc(C=C)c56)c5cncc4-5)[nH]cc3c12] | V-548 |
| 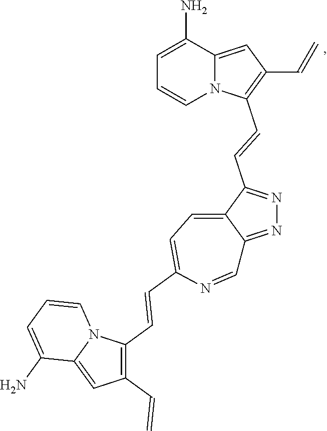 [SMILES: C=Cc1cc2c(N)cccn2c1/C=C/c1ccc2c(/C=C/c3c(C=C)cc4c(N)cccn34)nnc-2cn1] | V-549 |
| 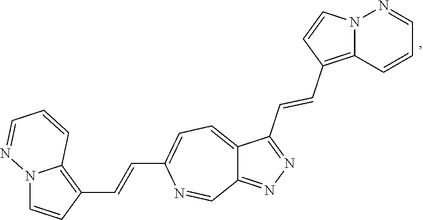 [SMILES: C(=C\c1ccn2ncccc12)/c1ccc2c(/C=C/c3ccn4ncccc34)nnc-2cn1] | V-550 |
| 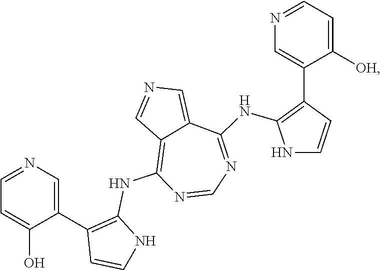 [SMILES: Oc1ccncc1-c1cc[nH]c1Nc1ncnc(Nc2[nH]ccc2-c2cnccc2O)c2cncc1-2] | V-551 |
| 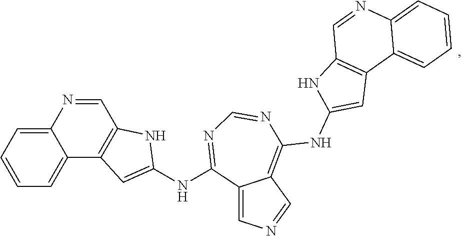 [SMILES: c1ccc2c(c1)ncc1[nH]c(Nc3ncnc(Nc4cc5c(cnc6ccccc65)[nH]4)c4cncc3-4)cc12] | V-552 |
| 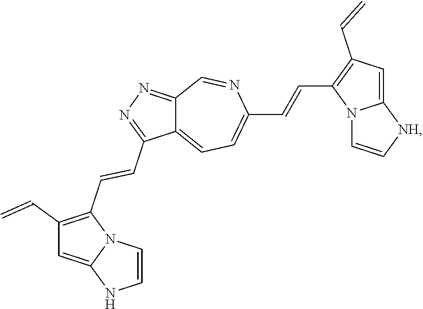 [SMILES: C=Cc1cc2[nH]ccn2c1/C=C/c1ccc2c(/C=C/c3c(C=C)cc4[nH]ccn34)nnc-2cn1] | V-553 |
| 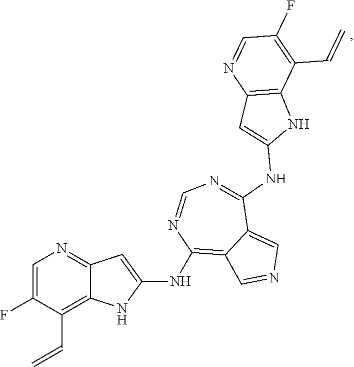 [SMILES: C=Cc1c(F)cnc2cc(Nc3ncnc(Nc4cc5ncc(F)c(C=C)c5[nH]4)c4cncc3-4)[nH]c12] | V-554 |
| 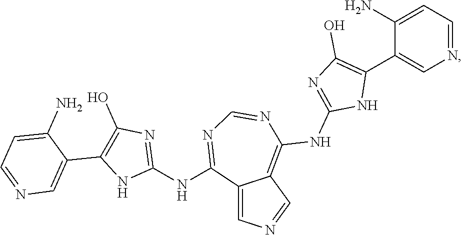 [SMILES: Nc1ccncc1-c1[nH]c(Nc2ncnc(Nc3nc(O)c(-c4cnccc4N)[nH]3)c3cncc2-3)nc1O] | V-555 |
| 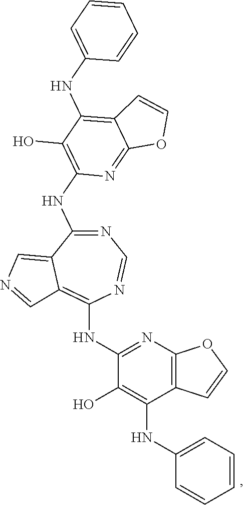 [SMILES: Oc1c(Nc2ncnc(Nc3nc4occc4c(Nc4ccccc4)c3O)c3cncc2-3)nc2occc2c1Nc1ccccc1] | V-556 |
| 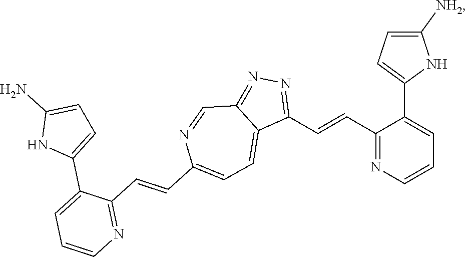 [SMILES: Nc1ccc(-c2cccnc2/C=C/c2ccc3c(/C=C/c4ncccc4-c4ccc(N)[nH]4)nnc-3cn2)[nH]1] | V-557 |
| 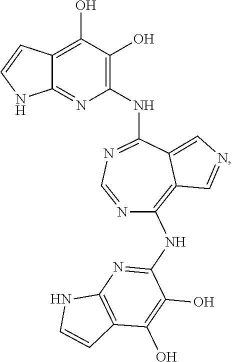 [SMILES: Oc1c(Nc2ncnc(Nc3nc4[nH]ccc4c(O)c3O)c3cncc2-3)nc2[nH]ccc2c1O] | V-558 |
| 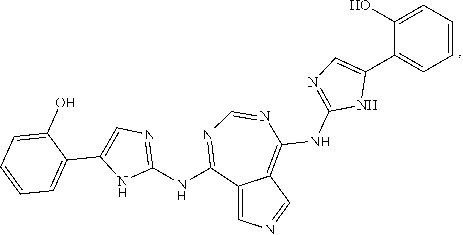 [SMILES: Oc1ccccc1-c1cnc(Nc2ncnc(Nc3ncc(-c4ccccc4O)[nH]3)c3cncc2-3)[nH]1] | V-559 |
| 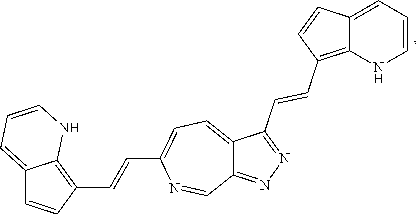 [SMILES: C(=C\c1ccc2ccc[nH]c1-2)/c1ccc2c(/C=C/c3ccc4ccc[nH]c3-4)nnc-2cn1] | V-560 |
| 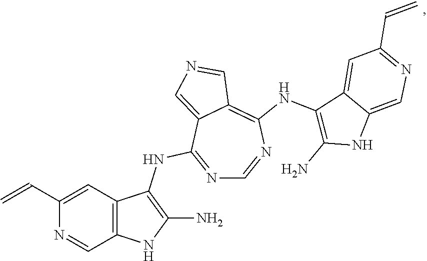 [SMILES: C=Cc1cc2c(Nc3ncnc(Nc4c(N)[nH]c5cnc(C=C)cc45)c4cncc3-4)c(N)[nH]c2cn1] | V-561 |
| 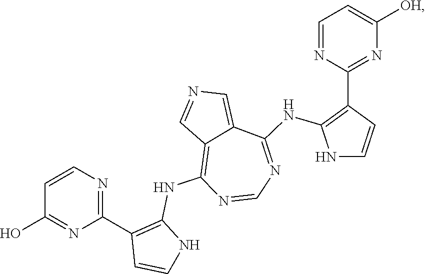 [SMILES: Oc1ccnc(-c2cc[nH]c2Nc2ncnc(Nc3[nH]ccc3-c3nccc(O)n3)c3cncc2-3)n1] | V-562 |
| 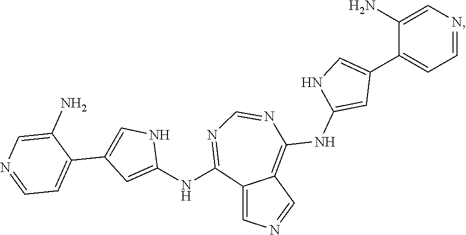 [SMILES: Nc1cnccc1-c1c[nH]c(Nc2ncnc(Nc3cc(-c4ccncc4N)c[nH]3)c3cncc2-3)c1] | V-563 |
| 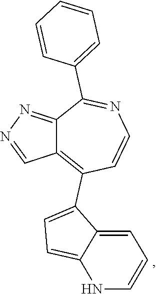 [SMILES: c1ccc(-c2nccc(-c3ccc4[nH]cccc3-4)c3cnnc2-3)cc1] | V-564 |
| 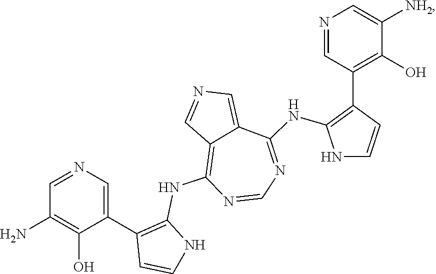 [SMILES: Nc1cncc(-c2cc[nH]c2Nc2ncnc(Nc3[nH]ccc3-c3cncc(N)c3O)c3cncc2-3)c1O] | V-565 |
| 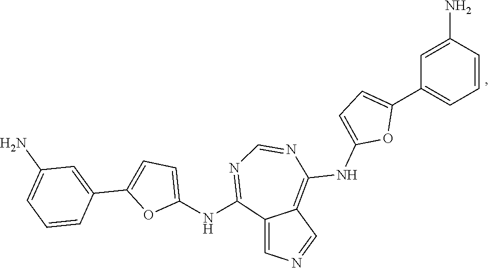 [SMILES: Nc1cccc(-c2ccc(Nc3ncnc(Nc4ccc(-c5cccc(N)c5)o4)c4cncc3-4)o2)c1] | V-566 |
| 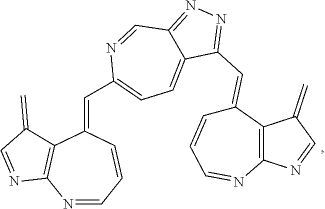 [SMILES: C=c1cnc2nccc/c(=C\c3ccc4c(/C=c5\cccnc6ncc(=C)c5=6)nnc-4cn3)c1=2] | V-567 |
| 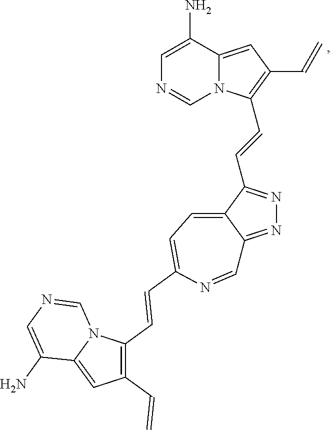 [SMILES: C=Cc1cc2c(N)cncn2c1/C=C/c1ccc2c(/C=C/c3c(C=C)cc4c(N)cncn34)nnc-2cn1] | V-568 |
| 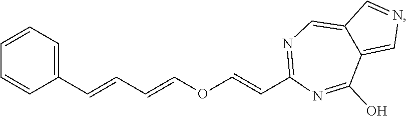 [SMILES: Oc1nc(/C=C/O/C=C/C=C/c2ccccc2)ncc2cncc1-2] | V-569 |
| 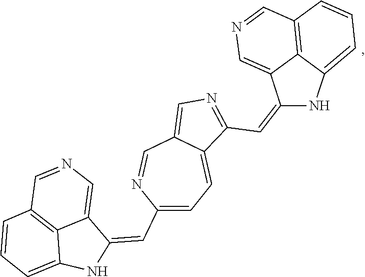 [SMILES: C(/c1ccc2c(/C=c3/[nH]c4cccc5cncc3c54)ncc-2cn1)=c1\[nH]c2cccc3cncc1c32] | V-570 |
| 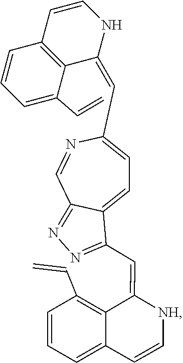 [SMILES: C=Cc1cccc2c1/C(=C\c1ccc3c(/C=C4/NC=Cc5cccc(C=C)c54)nnc-3cn1)NC=C2] | V-571 |
| 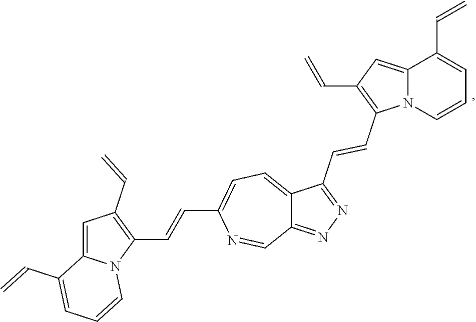 [SMILES: C=Cc1cc2c(C=C)cccn2c1/C=C/c1ccc2c(/C=C/c3c(C=C)cc4c(C=C)cccn34)nnc-2cn1] | V-572 |
| 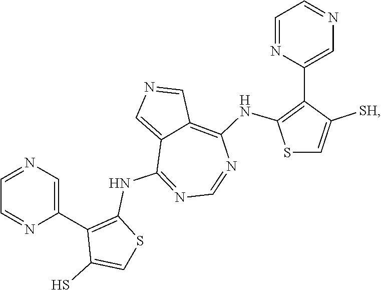 [SMILES: Sc1csc(Nc2ncnc(Nc3scc(S)c3-c3cnccn3)c3cncc2-3)c1-c1cnccn1] | V-573 |
| 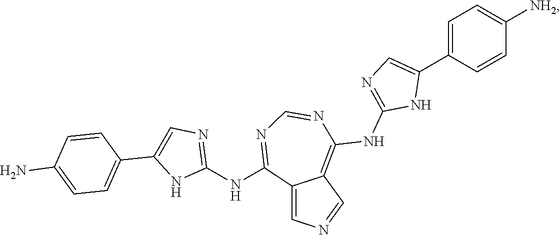 [SMILES: Nc1ccc(-c2cnc(Nc3ncnc(Nc4ncc(-c5ccc(N)cc5)[nH]4)c4cncc3-4)[nH]2)cc1] | V-574 |
| 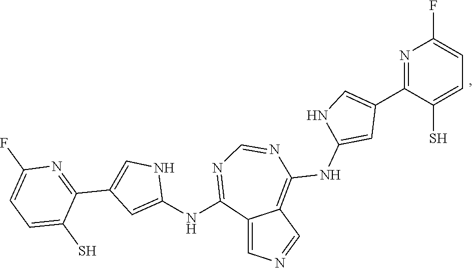 [SMILES: Fc1ccc(S)c(-c2c[nH]c(Nc3ncnc(Nc4cc(-c5nc(F)ccc5S)c[nH]4)c4cncc3-4)c2)n1] | V-575 |
| 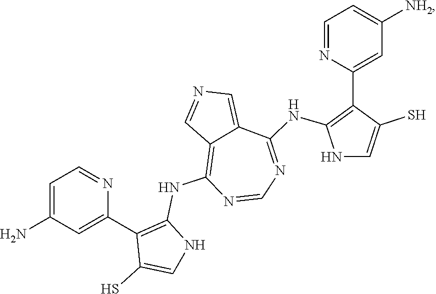 [SMILES: Nc1ccnc(-c2c(S)c[nH]c2Nc2ncnc(Nc3[nH]cc(S)c3-c3cc(N)ccn3)c3cncc2-3)c1] | V-576 |
| 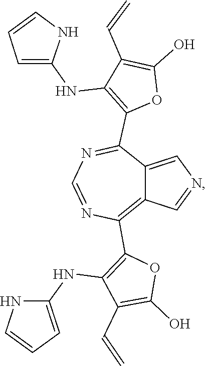 [SMILES: C=Cc1c(O)oc(-c2ncnc(-c3oc(O)c(C=C)c3Nc3ccc[nH]3)c3cncc2-3)c1Nc1ccc[nH]1] | V-577 |
| 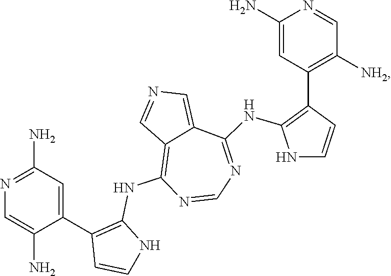 [SMILES: Nc1cc(-c2cc[nH]c2Nc2ncnc(Nc3[nH]ccc3-c3cc(N)ncc3N)c3cncc2-3)c(N)cn1] | V-578 |
| 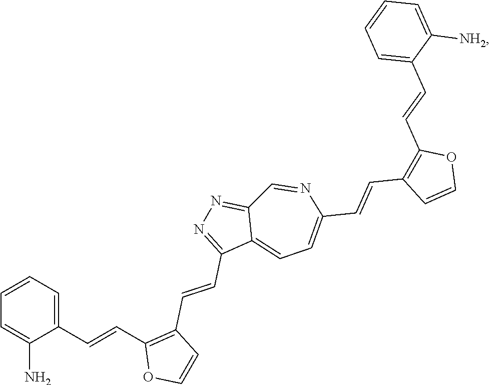 [SMILES: Nc1ccccc1/C=C/c1occc1/C=C/c1ccc2c(/C=C/c3ccoc3/C=C/c3ccccc3N)nnc-2cn1] | V-579 |
| 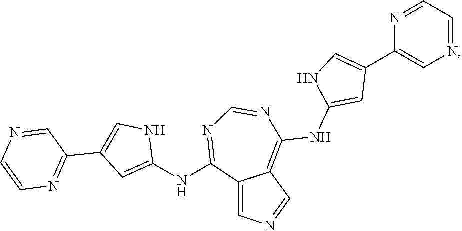 [SMILES: c1cnc(-c2c[nH]c(Nc3ncnc(Nc4cc(-c5cnccn5)c[nH]4)c4cncc3-4)c2)cn1] | V-580 |
| 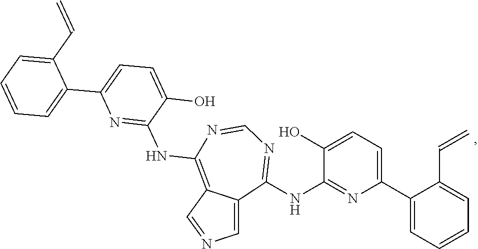 [SMILES: C=Cc1ccccc1-c1ccc(O)c(Nc2ncnc(Nc3nc(-c4ccccc4C=C)ccc3O)c3cncc2-3)n1] | V-581 |
| 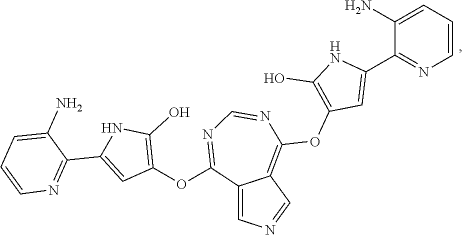 [SMILES: Nc1cccnc1-c1cc(Oc2ncnc(Oc3cc(-c4ncccc4N)[nH]c3O)c3cncc2-3)c(O)[nH]1] | V-582 |
| 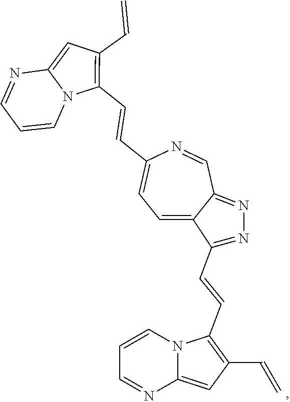 [SMILES: C=Cc1cc2ncccn2c1/C=C/c1ccc2c(/C=C/c3c(C=C)cc4ncccn34)nnc-2cn1] | V-583 |
| 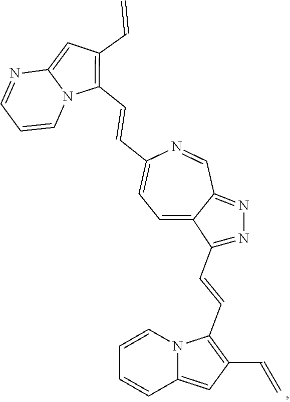 [SMILES: C=Cc1cc2ccccn2c1/C=C/c1nnc2cnc(/C=C/c3c(C=C)cc4ncccn34)ccc1-2] | V-584 |
| 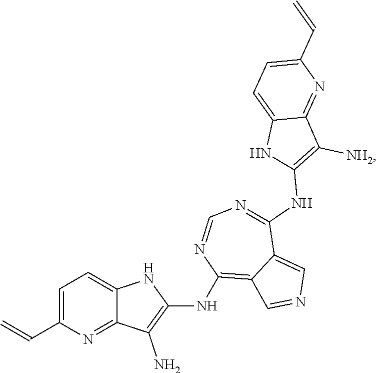 [SMILES: C=Cc1ccc2[nH]c(Nc3ncnc(Nc4[nH]c5ccc(C=C)nc5c4N)c4cncc3-4)c(N)c2n1] | V-585 |
| 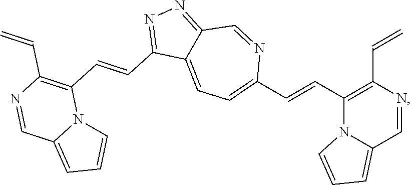 [SMILES: C=Cc1ncc2cccn2c1/C=C/c1ccc2c(/C=C/c3c(C=C)ncc4cccn34)nnc-2cn1] | V-586 |
| 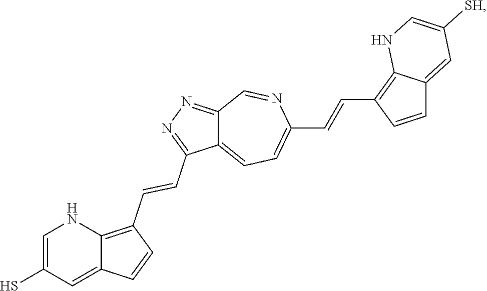 [SMILES: Sc1c[nH]c2c(/C=C/c3ccc4c(/C=C/c5ccc6cc(S)c[nH]c5-6)nnc-4cn3)ccc-2c1] | V-587 |
| 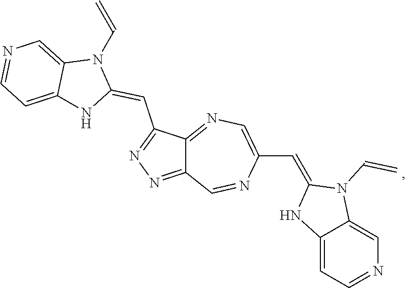 [SMILES: C=CN1/C(=C/c2cnc3c(/C=C4\Nc5ccncc5N4C=C)nnc-3cn2)Nc2ccncc21] | V-588 |
| 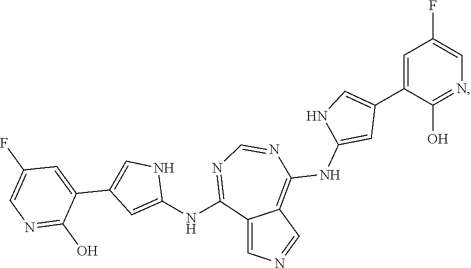 [SMILES: Oc1ncc(F)cc1-c1c[nH]c(Nc2ncnc(Nc3cc(-c4cc(F)cnc4O)c[nH]3)c3cncc2-3)c1] | V-589 |
| 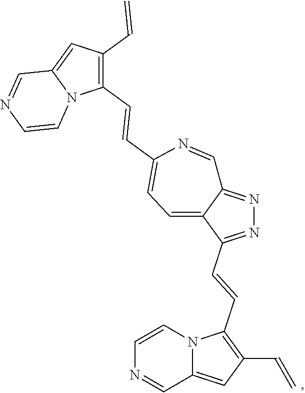 [SMILES: C=Cc1cc2cnccn2c1/C=C/c1ccc2c(/C=C/c3c(C=C)cc4cnccn34)nnc-2cn1] | V-590 |
| 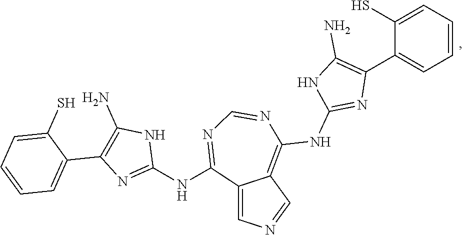 [SMILES: Nc1[nH]c(Nc2ncnc(Nc3nc(-c4ccccc4S)c(N)[nH]3)c3cncc2-3)nc1-c1ccccc1S] | V-591 |
| 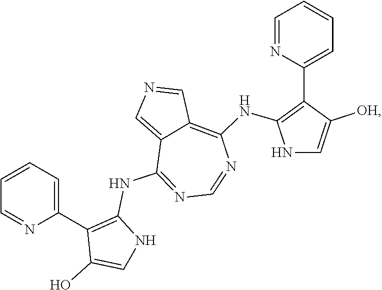 [SMILES: Oc1c[nH]c(Nc2ncnc(Nc3[nH]cc(O)c3-c3ccccn3)c3cncc2-3)c1-c1ccccn1] | V-592 |
| 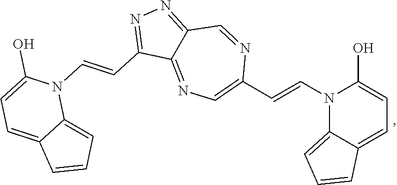 [SMILES: Oc1ccc2cccc-2n1/C=C/c1cnc2c(/C=C/n3c(O)ccc4cccc3-4)nnc-2cn1] | V-593 |
| 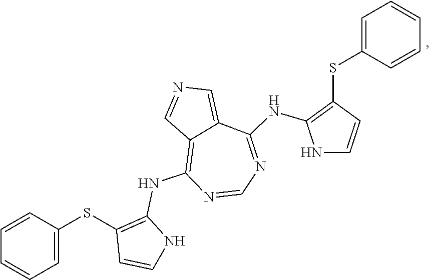 [SMILES: c1ccc(Sc2cc[nH]c2Nc2ncnc(Nc3[nH]ccc3Sc3ccccc3)c3cncc2-3)cc1] | V-594 |
| 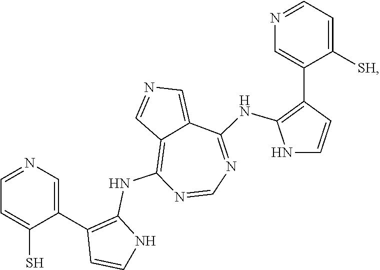 [SMILES: Sc1ccncc1-c1cc[nH]c1Nc1ncnc(Nc2[nH]ccc2-c2cnccc2S)c2cncc1-2] | V-595 |
| 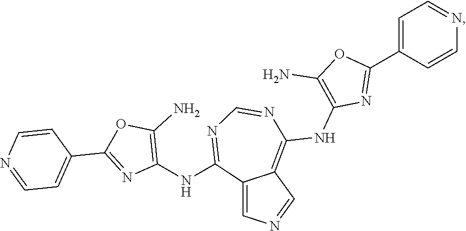 [SMILES: Nc1oc(-c2ccncc2)nc1Nc1ncnc(Nc2nc(-c3ccncc3)oc2N)c2cncc1-2] | V-596 |
| 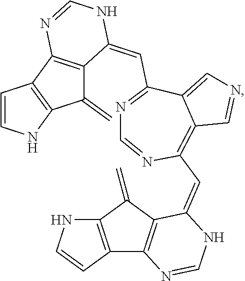 [SMILES: C=c1c2/c(=C\c3ncnc(/C=c4/[nH]cnc5c6cc[nH]c6c(=C)c4=5)c4cncc3-4)[nH]cnc=2c2cc[nH]c12] | V-597 |
| 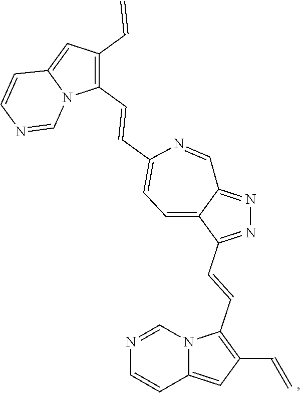 [SMILES: C=Cc1cc2ccncn2c1/C=C/c1ccc2c(/C=C/c3c(C=C)cc4ccncn34)nnc-2cn1] | V-598 |
| 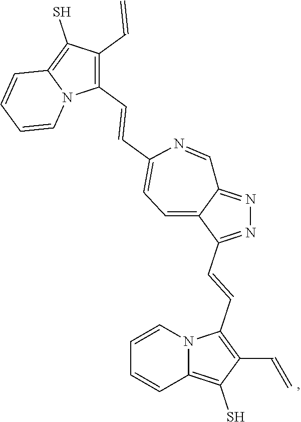 [SMILES: C=Cc1c(S)c2ccccn2c1/C=C/c1ccc2c(/C=C/c3c(C=C)c(S)c4ccccn34)nnc-2cn1] | V-599 |
| 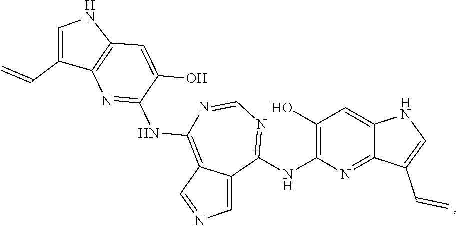 [SMILES: C=Cc1c[nH]c2cc(O)c(Nc3ncnc(Nc4nc5c(C=C)c[nH]c5cc4O)c4cncc3-4)nc12] | V-600 |
| 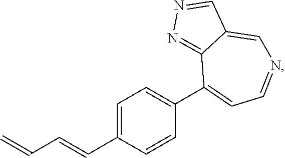 [SMILES: C=C/C=C/c1ccc(-c2ccncc3cnnc2-3)cc1] | V-601 |
| 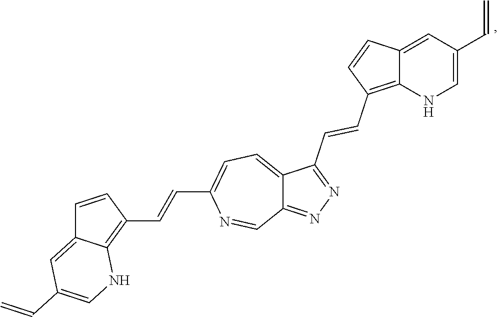 [SMILES: C=Cc1c[nH]c2c(/C=C/c3ccc4c(/C=C/c5ccc6cc(C=C)c[nH]c5-6)nnc-4cn3)ccc-2c1] | V-602 |
| 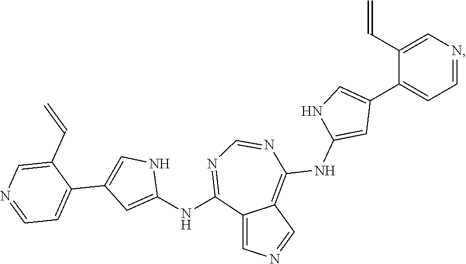 [SMILES: C=Cc1cnccc1-c1c[nH]c(Nc2ncnc(Nc3cc(-c4ccncc4C=C)c[nH]3)c3cncc2-3)c1] | V-603 |
| 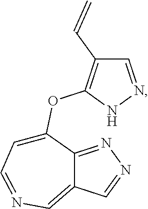 [SMILES: C=Cc1cn[nH]c1Oc1ccncc2cnnc1-2] | V-604 |
| 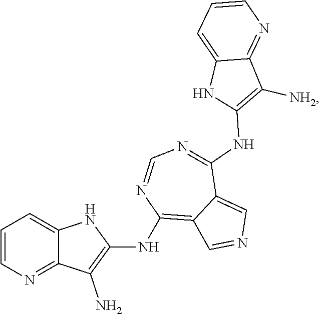 [SMILES: Nc1c(Nc2ncnc(Nc3[nH]c4cccnc4c3N)c3cncc2-3)[nH]c2cccnc12] | V-605 |
| 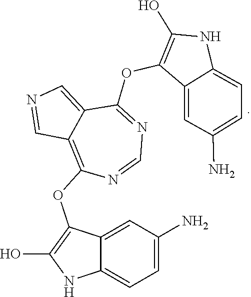 [SMILES: Nc1ccc2[nH]c(O)c(Oc3ncnc(Oc4c(O)[nH]c5ccc(N)cc45)c4cncc3-4)c2c1] | V-606 |
| 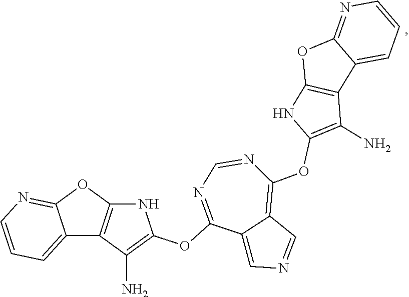 [SMILES: Nc1c(Oc2ncnc(Oc3[nH]c4oc5ncccc5c4c3N)c3cncc2-3)[nH]c2oc3ncccc3c12] | V-607 |
| 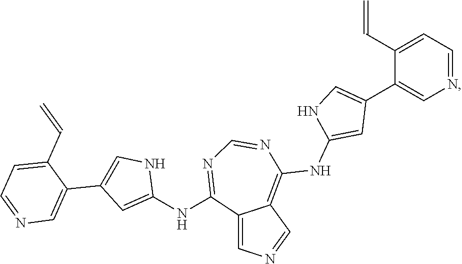 [SMILES: C=Cc1ccncc1-c1c[nH]c(Nc2ncnc(Nc3cc(-c4cnccc4C=C)c[nH]3)c3cncc2-3)c1] | V-608 |
| 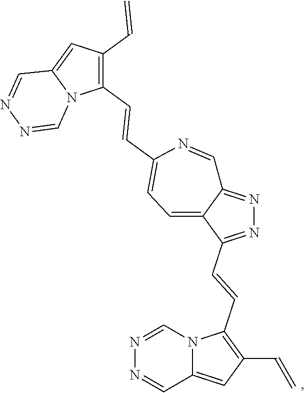 [SMILES: C=Cc1cc2cnncn2c1/C=C/c1ccc2c(/C=C/c3c(C=C)cc4cnncn34)nnc-2cn1] | V-609 |
| 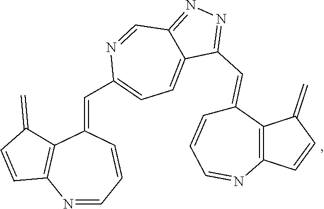 [SMILES: C=c1ccc2nccc/c(=C\c3ccc4c(/C=c5\cccnc6ccc(=C)c5=6)nnc-4cn3)c1=2] | V-610 |
| 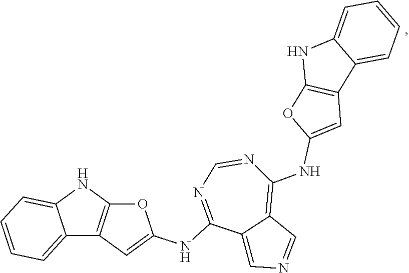 [SMILES: c1ccc2c(c1)[nH]c1oc(Nc3ncnc(Nc4cc5c([nH]c6ccccc65)o4)c4cncc3-4)cc12] | V-611 |
| 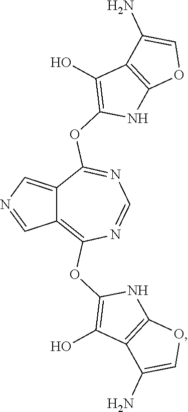 [SMILES: Nc1coc2[nH]c(Oc3ncnc(Oc4[nH]c5occ(N)c5c4O)c4cncc3-4)c(O)c12] | V-612 |
| 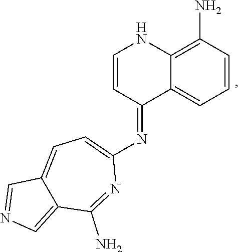 [SMILES: Nc1nc(/N=c2\cc[nH]c3c(N)cccc23)ccc2cncc1-2] | V-613 |
| 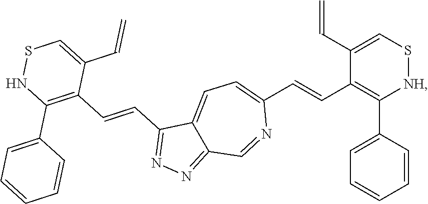 [SMILES: C=CC1=CSNC(c2ccccc2)=C1/C=C/c1ccc2c(/C=C/C3=C(c4ccccc4)NSC=C3C=C)nnc-2cn1] | V-614 |
| 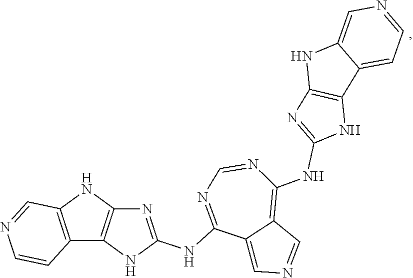 [SMILES: c1cc2c(cn1)[nH]c1nc(Nc3ncnc(Nc4nc5[nH]c6cnccc6c5[nH]4)c4cncc3-4)[nH]c12] | V-615 |
| 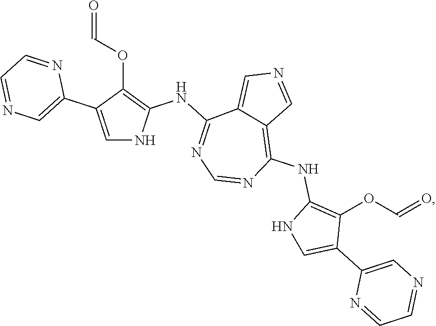 [SMILES: O=COc1c(-c2cnccn2)c[nH]c1Nc1ncnc(Nc2[nH]cc(-c3cnccn3)c2OC=O)c2cncc1-2] | V-616 |
| 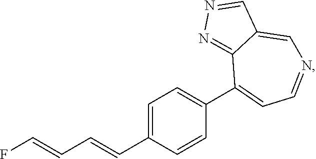 [SMILES: F/C=C/C=C/c1ccc(-c2ccncc3cnnc2-3)cc1] | V-617 |
| 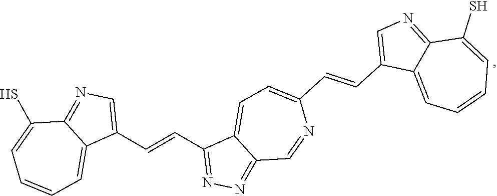 [SMILES: Sc1ccccc2c(/C=C/c3ccc4c(/C=C/c5cnc6c(S)ccccc5-6)nnc-4cn3)cnc1-2] | V-618 |
| 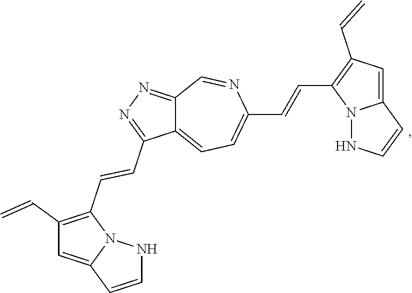 [SMILES: C=Cc1cc2cc[nH]n2c1/C=C/c1ccc2c(/C=C/c3c(C=C)cc4cc[nH]n34)nnc-2cn1] | V-619 |
| 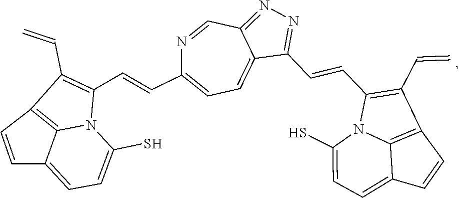 [SMILES: C=Cc1c(/C=C/c2ccc3c(/C=C/c4c(C=C)c5ccc6ccc(S)n4c65)nnc-3cn2)n2c(S)ccc3ccc1c32] | V-620 |
| 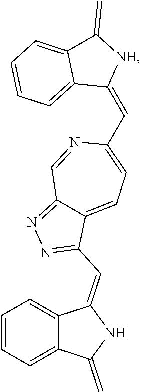 [SMILES: C=c1[nH]/c(=C/c2ccc3c(/C=c4/[nH]c(=C)c5ccccc45)nnc-3cn2)c2ccccc12] | V-621 |
| 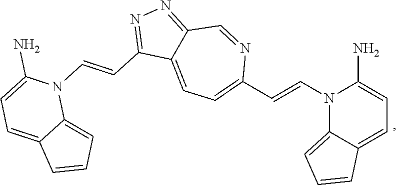 [SMILES: Nc1ccc2cccc-2n1/C=C/c1ccc2c(/C=C/n3c(N)ccc4cccc3-4)nnc-2cn1] | V-622 |
| 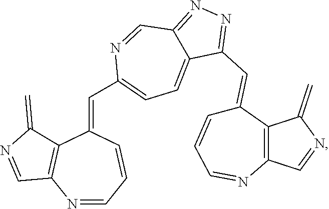 [SMILES: C=c1ncc2nccc/c(=C\c3ccc4c(/C=c5\cccnc6cnc(=C)c5=6)nnc-4cn3)c1=2] | V-623 |
| 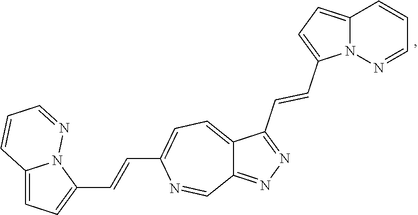 [SMILES: C(=C\c1ccc2cccnn12)/c1ccc2c(/C=C/c3ccc4cccnn34)nnc-2cn1] | V-624 |
| 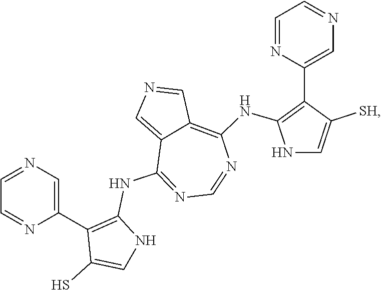 [SMILES: Sc1c[nH]c(Nc2ncnc(Nc3[nH]cc(S)c3-c3cnccn3)c3cncc2-3)c1-c1cnccn1] | V-625 |
| 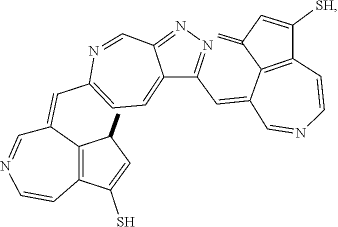 [SMILES: C=c1cc(S)c2ccnc/c(=C/c3nnc4cnc(/C=C5/C=NC=CC6=C5[C@@H](C)C=C6S)ccc3-4)c1=2] | V-626 |
| 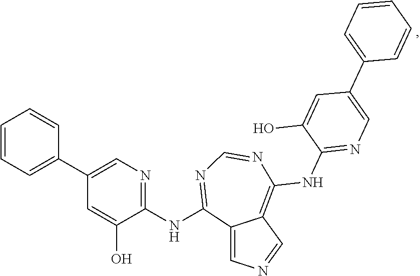 [SMILES: Oc1cc(-c2ccccc2)cnc1Nc1ncnc(Nc2ncc(-c3ccccc3)cc2O)c2cncc1-2] | V-627 |
| 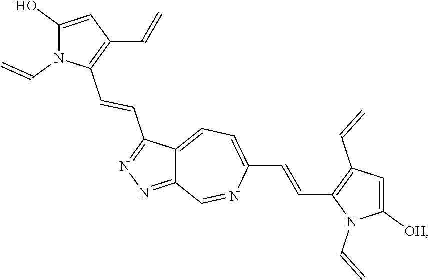 [SMILES: C=Cc1cc(O)n(C=C)c1/C=C/c1ccc2c(/C=C/c3c(C=C)cc(O)n3C=C)nnc-2cn1] | V-628 |
| 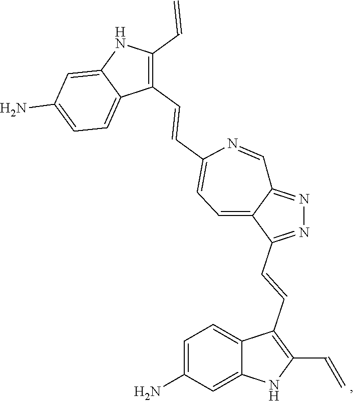 [SMILES: C=Cc1[nH]c2cc(N)ccc2c1/C=C/c1ccc2c(/C=C/c3c(C=C)[nH]c4cc(N)ccc34)nnc-2cn1] | V-629 |
| 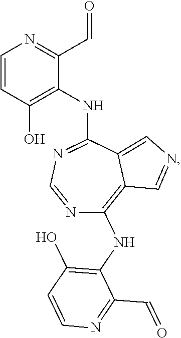 [SMILES: O=Cc1nccc(O)c1Nc1ncnc(Nc2c(O)ccnc2C=O)c2cncc1-2] | V-630 |
| 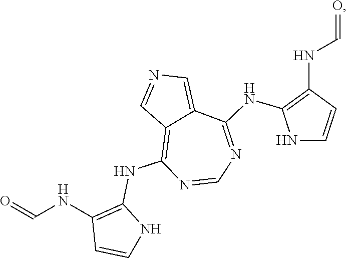 [SMILES: O=CNc1cc[nH]c1Nc1ncnc(Nc2[nH]ccc2NC=O)c2cncc1-2] | V-631 |
| 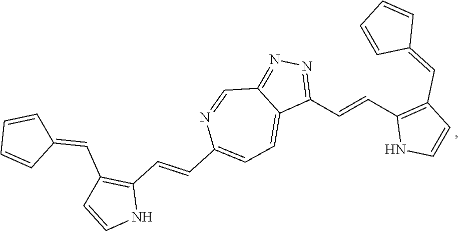 [SMILES: C1=CC(=Cc2cc[nH]c2/C=C/c2ccc3c(/C=C/c4[nH]ccc4C=C4C=CC=C4)nnc-3cn2)C=C1] | V-632 |
| 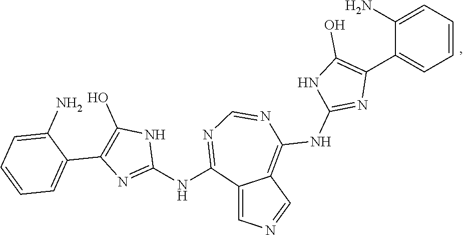 [SMILES: Nc1ccccc1-c1nc(Nc2ncnc(Nc3nc(-c4ccccc4N)c(O)[nH]3)c3cncc2-3)[nH]c1O] | V-633 |
| 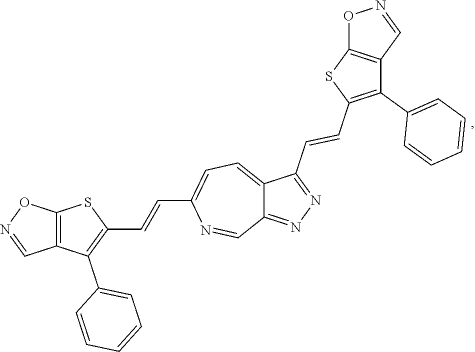 [SMILES: C(=C\c1sc2oncc2c1-c1ccccc1)/c1ccc2c(/C=C/c3sc4oncc4c3-c3ccccc3)nnc-2cn1] | V-634 |
| 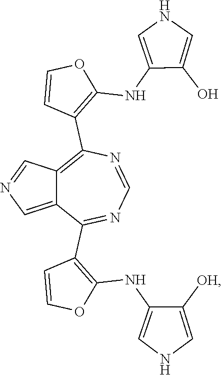 [SMILES: Oc1c[nH]cc1Nc1occc1-c1ncnc(-c2ccoc2Nc2c[nH]cc2O)c2cncc1-2] | V-635 |
| 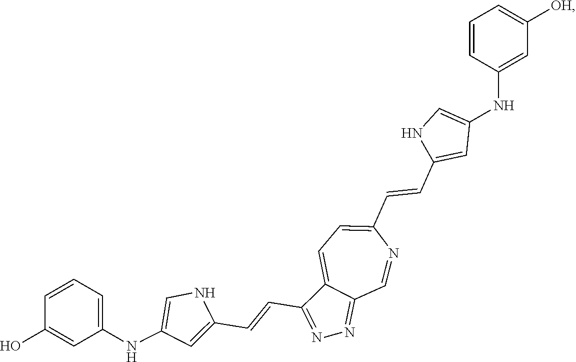 [SMILES: Oc1cccc(Nc2c[nH]c(/C=C/c3ccc4c(/C=C/c5cc(Nc6cccc(O)c6)c[nH]5)nnc-4cn3)c2)c1] | V-636 |
| 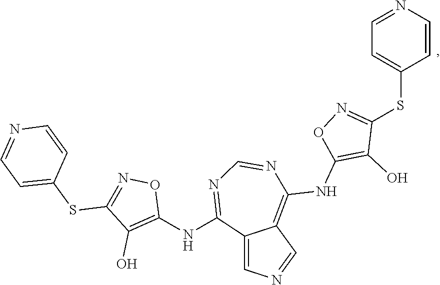 [SMILES: Oc1c(Sc2ccncc2)noc1Nc1ncnc(Nc2onc(Sc3ccncc3)c2O)c2cncc1-2] | V-637 |
| 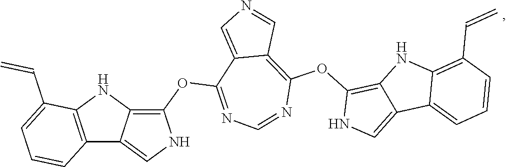 [SMILES: C=Cc1cccc2c1[nH]c1c(Oc3ncnc(Oc4[nH]cc5c4[nH]c4c(C=C)cccc45)c4cncc3-4)[nH]cc12] | V-638 |
| 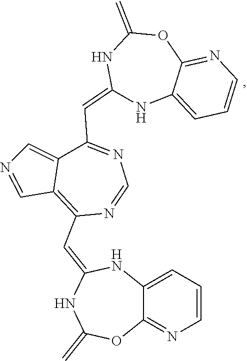 [SMILES: C=c1[nH]/c(=C/c2ncnc(/C=c3/[nH]c(=C)oc4ncccc4[nH]3)c3cncc2-3)[nH]c2cccnc2o1] | V-639 |
| 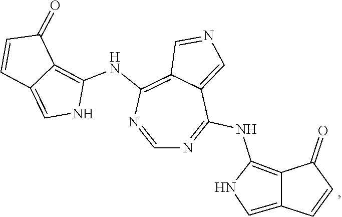 [SMILES: O=C1C=Cc2c[nH]c(Nc3ncnc(Nc4[nH]cc5c4C(=O)C=C5)c4cncc3-4)c21] | V-640 |
| 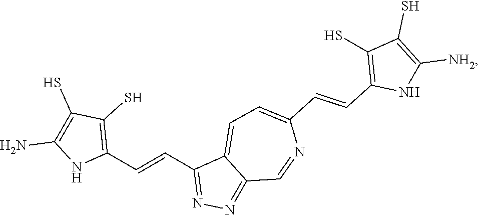 [SMILES: Nc1[nH]c(/C=C/c2ccc3c(/C=C/c4[nH]c(N)c(S)c4S)nnc-3cn2)c(S)c1S] | V-641 |
| 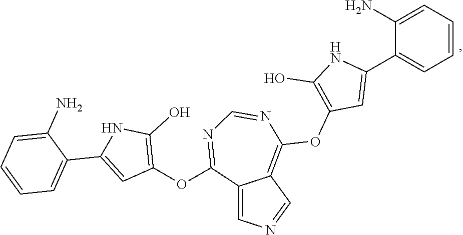 [SMILES: Nc1ccccc1-c1cc(Oc2ncnc(Oc3cc(-c4ccccc4N)[nH]c3O)c3cncc2-3)c(O)[nH]1] | V-642 |
| 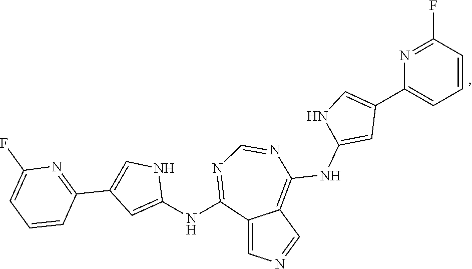 [SMILES: Fc1cccc(-c2c[nH]c(Nc3ncnc(Nc4cc(-c5cccc(F)n5)c[nH]4)c4cncc3-4)c2)n1] | V-643 |
| 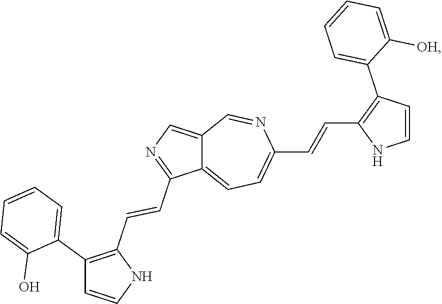 [SMILES: Oc1ccccc1-c1cc[nH]c1/C=C/c1ccc2c(/C=C/c3[nH]ccc3-c3ccccc3O)ncc-2cn1] | V-644 |
| 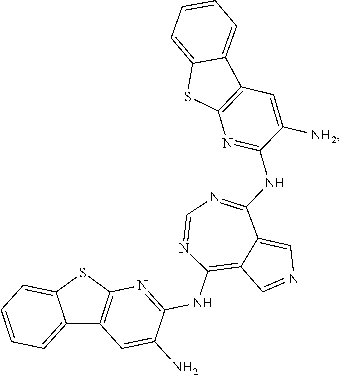 [SMILES: Nc1cc2c(nc1Nc1ncnc(Nc3nc4sc5ccccc5c4cc3N)c3cncc1-3)sc1ccccc12] | V-645 |
| 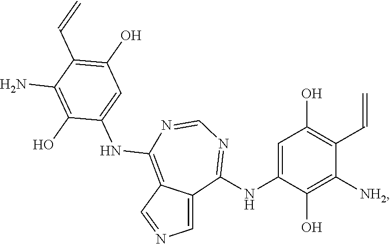 [SMILES: C=Cc1c(O)cc(Nc2ncnc(Nc3cc(O)c(C=C)c(N)c3O)c3cncc2-3)c(O)c1N] | V-646 |
| 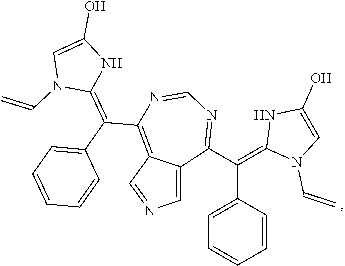 [SMILES: C=CN1C=C(O)N/C1=C(/c1ccccc1)c1ncnc(/C(=C2\NC(O)=CN2C=C)c2ccccc2)c2cncc1-2] | V-647 |
| 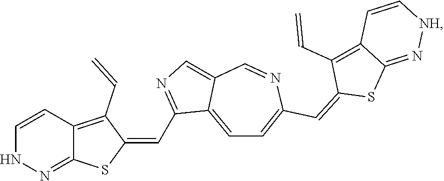 [SMILES: C=Cc1c2c(s/c1=C/c1ccc3c(/C=c4/sc5c(c4C=C)C=CNN=5)ncc-3cn1)=NNC=C2] | V-648 |
| 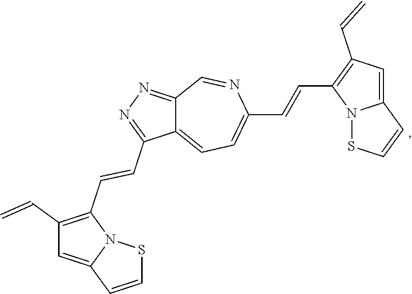 [SMILES: C=Cc1cc2ccsn2c1/C=C/c1ccc2c(/C=C/c3c(C=C)cc4ccsn34)nnc-2cn1] | V-649 |
| 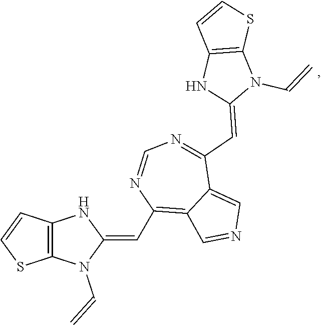 [SMILES: C=CN1/C(=C/c2ncnc(/C=C3\Nc4ccsc4N3C=C)c3cncc2-3)Nc2ccsc21] | V-650 |
| 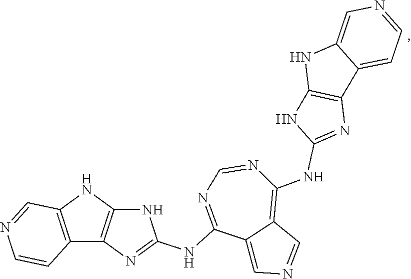 [SMILES: c1cc2c(cn1)[nH]c1[nH]c(Nc3ncnc(Nc4nc5c([nH]4)[nH]c4cnccc45)c4cncc3-4)nc12] | V-651 |
| 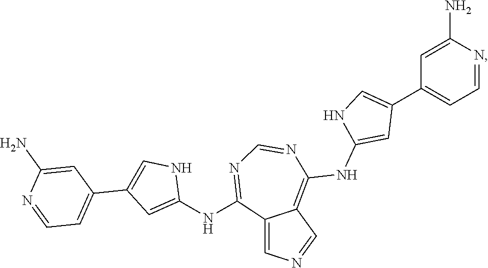 [SMILES: Nc1cc(-c2c[nH]c(Nc3ncnc(Nc4cc(-c5ccnc(N)c5)c[nH]4)c4cncc3-4)c2)ccn1] | V-652 |
| 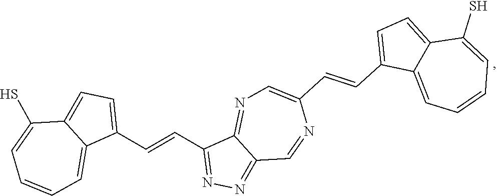 [SMILES: Sc1ccccc2c(/C=C/c3cnc4c(/C=C/c5ccc6c(S)ccccc5-6)nnc-4cn3)ccc1-2] | V-653 |
| 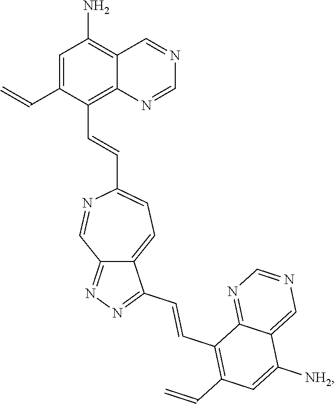 [SMILES: C=Cc1cc(N)c2cncnc2c1/C=C/c1ccc2c(/C=C/c3c(C=C)cc(N)c4cncnc34)nnc-2cn1] | V-654 |
| 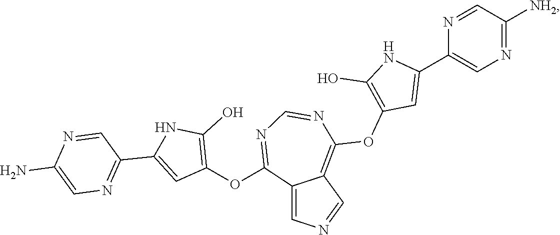 [SMILES: Nc1cnc(-c2cc(Oc3ncnc(Oc4cc(-c5cnc(N)cn5)[nH]c4O)c4cncc3-4)c(O)[nH]2)cn1] | V-655 |
| 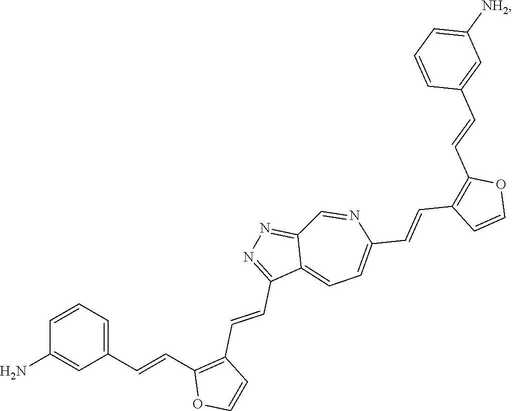 [SMILES: Nc1cccc(/C=C/c2occc2/C=C/c2ccc3c(/C=C/c4ccoc4/C=C/c4cccc(N)c4)nnc-3cn2)c1] | V-656 |
| 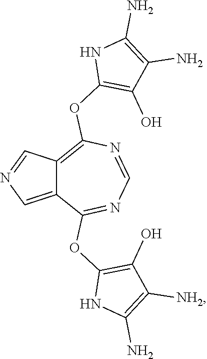 [SMILES: Nc1[nH]c(Oc2ncnc(Oc3[nH]c(N)c(N)c3O)c3cncc2-3)c(O)c1N] | V-657 |
| 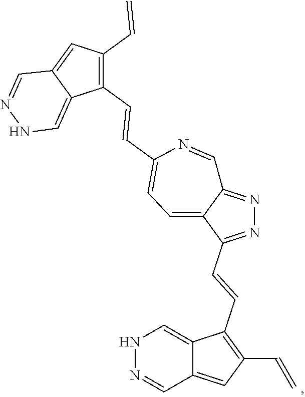 [SMILES: C=Cc1cc2cn[nH]cc-2c1/C=C/c1ccc2c(/C=C/c3c(C=C)cc4cn[nH]cc3-4)nnc-2cn1] | V-658 |
| 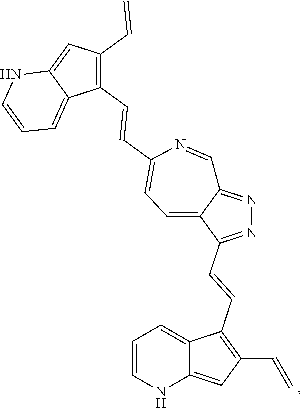 [SMILES: C=Cc1cc2[nH]cccc-2c1/C=C/c1ccc2c(/C=C/c3c(C=C)cc4[nH]cccc3-4)nnc-2cn1] | V-659 |
| 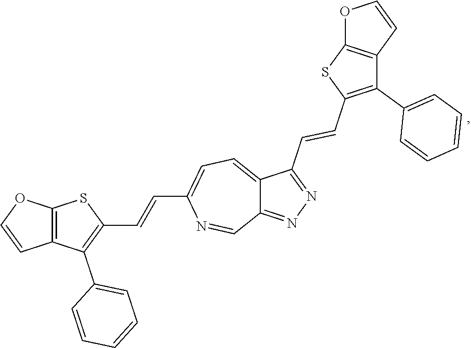 [SMILES: C(=C\c1sc2occc2c1-c1ccccc1)/c1ccc2c(/C=C/c3sc4occc4c3-c3ccccc3)nnc-2cn1] | V-660 |
| 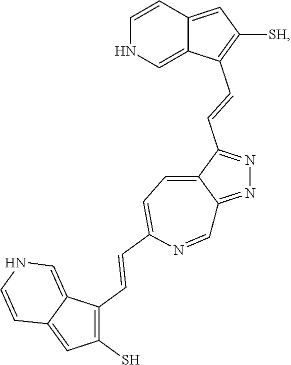 [SMILES: Sc1cc2cc[nH]cc-2c1/C=C/c1ccc2c(/C=C/c3c(S)cc4cc[nH]cc3-4)nnc-2cn1] | V-661 |
| 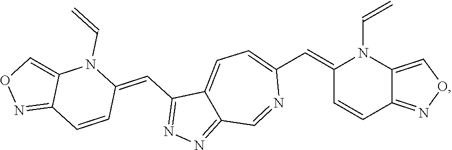 [SMILES: C=CN1/C(=C/c2ccc3c(/C=C4\C=Cc5nocc5N4C=C)nnc-3cn2)C=Cc2nocc21] | V-662 |
| 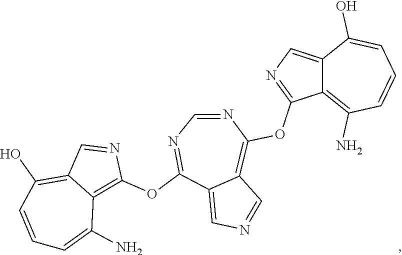 [SMILES: Nc1cccc(O)c2cnc(Oc3ncnc(Oc4ncc5c(O)cccc(N)c4-5)c4cncc3-4)c1-2] | V-663 |
| 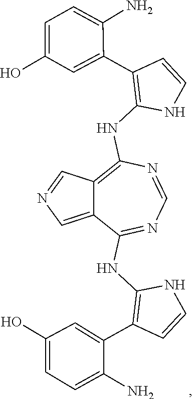 [SMILES: Nc1ccc(O)cc1-c1cc[nH]c1Nc1ncnc(Nc2[nH]ccc2-c2cc(O)ccc2N)c2cncc1-2] | V-664 |
| 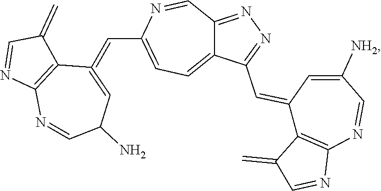 | V-665 |
| 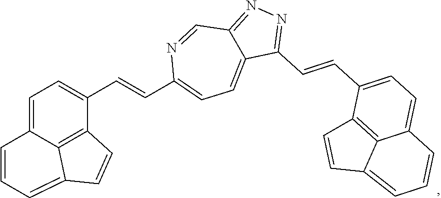 [SMILES: C1=Cc2c(/C=C/c3ccc4c(/C=C/c5ccc6cccc7c6c5C=C7)nnc-4cn3)ccc3cccc1c23] | V-666 |
| 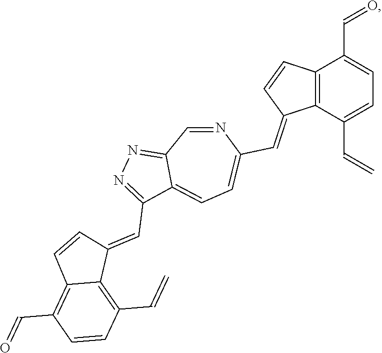 [SMILES: C=Cc1ccc(C=O)c2c1/C(=C/c1ccc3c(/C=C4\C=Cc5c(C=O)ccc(C=C)c54)nnc-3cn1)C=C2] | V-667 |
| 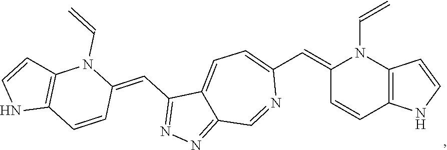 [SMILES: C=CN1/C(=C/c2ccc3c(/C=C4\C=Cc5[nH]ccc5N4C=C)nnc-3cn2)C=Cc2[nH]ccc21] | V-668 |
| 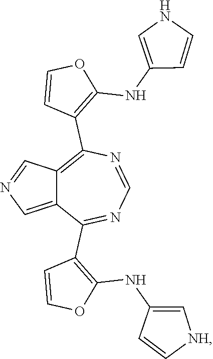 [SMILES: c1nc(-c2ccoc2Nc2cc[nH]c2)c2cncc-2c(-c2ccoc2Nc2cc[nH]c2)n1] | V-669 |
| 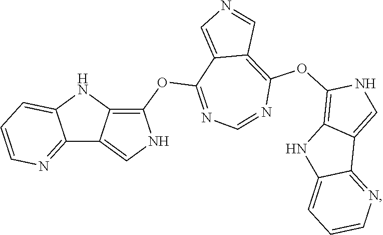 [SMILES: c1cnc2c(c1)[nH]c1c(Oc3ncnc(Oc4[nH]cc5c4[nH]c4cccnc45)c4cncc3-4)[nH]cc12] | V-670 |
| 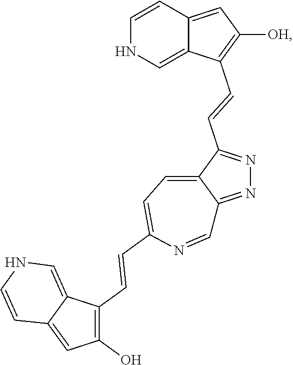 [SMILES: Oc1cc2cc[nH]cc-2c1/C=C/c1ccc2c(/C=C/c3c(O)cc4cc[nH]cc3-4)nnc-2cn1] | V-671 |
| 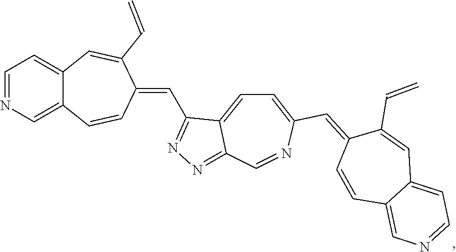 [SMILES: C=CC1=Cc2ccncc2C=C/C1=C\c1ccc2c(/C=C3\C=Cc4cnccc4C=C3C=C)nnc-2cn1] | V-672 |
| 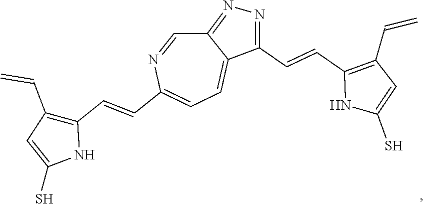 [SMILES: C=Cc1cc(S)[nH]c1/C=C/c1ccc2c(/C=C/c3[nH]c(S)cc3C=C)nnc-2cn1] | V-673 |
| 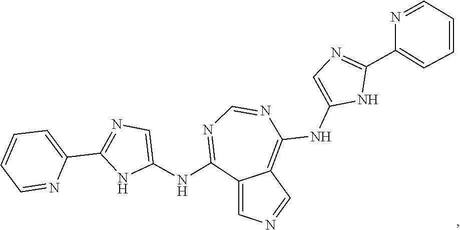 [SMILES: c1ccc(-c2ncc(Nc3ncnc(Nc4cnc(-c5ccccn5)[nH]4)c4cncc3-4)[nH]2)nc1] | V-674 |
| 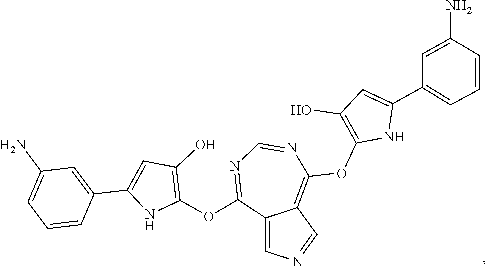 [SMILES: Nc1cccc(-c2cc(O)c(Oc3ncnc(Oc4[nH]c(-c5cccc(N)c5)cc4O)c4cncc3-4)[nH]2)c1] | V-675 |
| 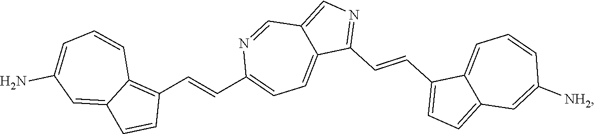 [SMILES: Nc1cccc2c(/C=C/c3ccc4c(/C=C/c5ccc6cc(N)cccc5-6)ncc-4cn3)ccc-2c1] | V-676 |
| 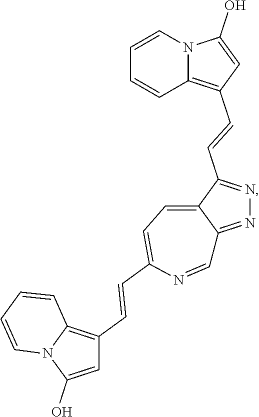 [SMILES: Oc1cc(/C=C/c2ccc3c(/C=C/c4cc(O)n5ccccc45)nnc-3cn2)c2ccccn12] | V-677 |
| 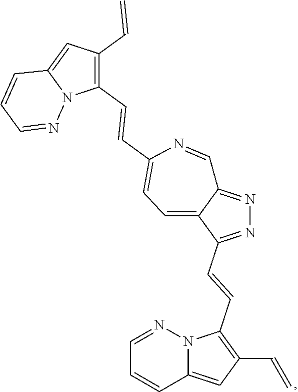 [SMILES: C=Cc1cc2cccnn2c1/C=C/c1ccc2c(/C=C/c3c(C=C)cc4cccnn34)nnc-2cn1] | V-678 |
| 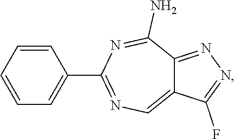 [SMILES: Nc1nc(-c2ccccc2)ncc2c(F)nnc1-2] | V-679 |
| 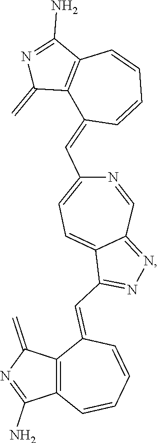 [SMILES: C=c1nc(N)c2cccc/c(=C\c3ccc4c(/C=c5\ccccc6c(N)nc(=C)c5=6)nnc-4cn3)c1=2] | V-680 |
| 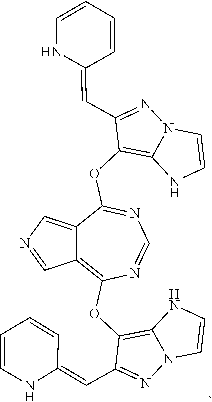 [SMILES: C1=CN/C(=C/c2nn3cc[nH]c3c2Oc2ncnc(Oc3c(/C=C4\C=CC=CN4)nn4cc[nH]c34)c3cncc2-3)C=C1] | V-681 |
| 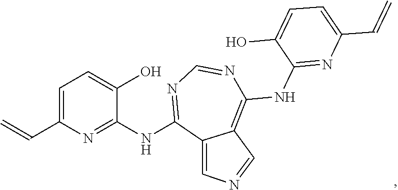 [SMILES: C=Cc1ccc(O)c(Nc2ncnc(Nc3nc(C=C)ccc3O)c3cncc2-3)n1] | V-682 |
| 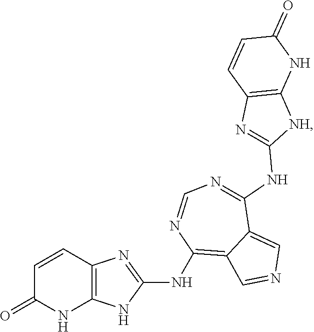 [SMILES: O=c1ccc2nc(Nc3ncnc(Nc4nc5ccc(=O)[nH]c5[nH]4)c4cncc3-4)[nH]c2[nH]1] | V-683 |
| 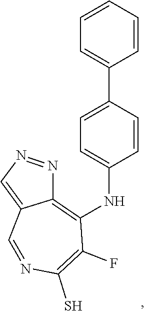 [SMILES: Fc1c(S)ncc2cnnc-2c1Nc1ccc(-c2ccccc2)cc1] | V-684 |
| 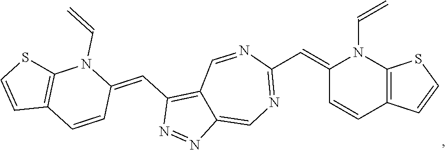 [SMILES: C=CN1/C(=C/c2ncc3nnc(/C=C4\C=Cc5ccsc5N4C=C)c-3cn2)C=Cc2ccsc21] | V-685 |
| 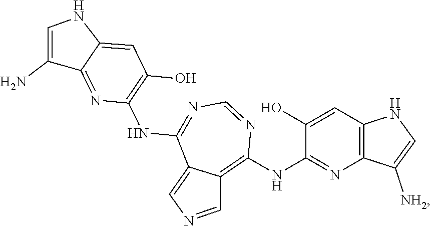 [SMILES: Nc1c[nH]c2cc(O)c(Nc3ncnc(Nc4nc5c(N)c[nH]c5cc4O)c4cncc3-4)nc12] | V-686 |
| 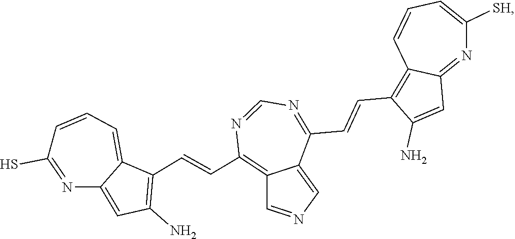 [SMILES: Nc1cc2nc(S)cccc-2c1/C=C/c1ncnc(/C=C/c2c(N)cc3nc(S)cccc2-3)c2cncc1-2] | V-687 |
| 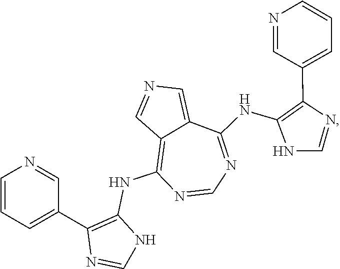 [SMILES: c1cncc(-c2nc[nH]c2Nc2ncnc(Nc3[nH]cnc3-c3cccnc3)c3cncc2-3)c1] | V-688 |
| 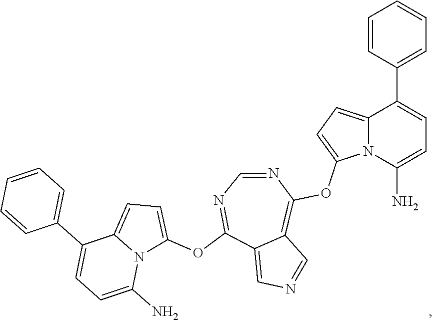 [SMILES: Nc1ccc(-c2ccccc2)c2ccc(Oc3ncnc(Oc4ccc5c(-c6ccccc6)ccc(N)n45)c4cncc3-4)n12] | V-689 |
| 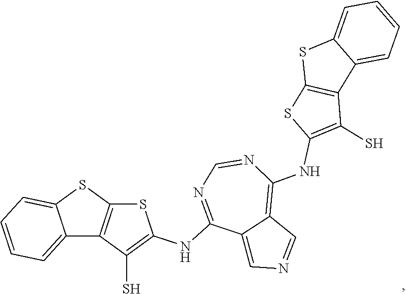 [SMILES: Sc1c(Nc2ncnc(Nc3sc4sc5ccccc5c4c3S)c3cncc2-3)sc2sc3ccccc3c12] | V-690 |
| 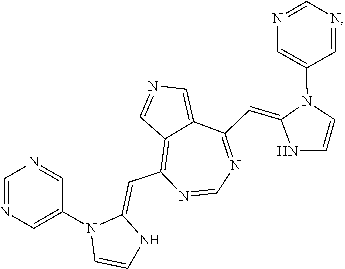 [SMILES: C1=CN(c2cncnc2)/C(=C/c2ncnc(/C=C3\NC=CN3c3cncnc3)c3cncc2-3)N1] | V-691 |
| 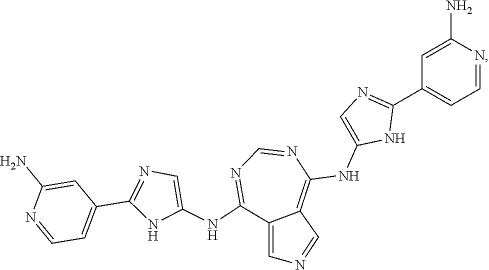 [SMILES: Nc1cc(-c2ncc(Nc3ncnc(Nc4cnc(-c5ccnc(N)c5)[nH]4)c4cncc3-4)[nH]2)ccn1] | V-692 |
| 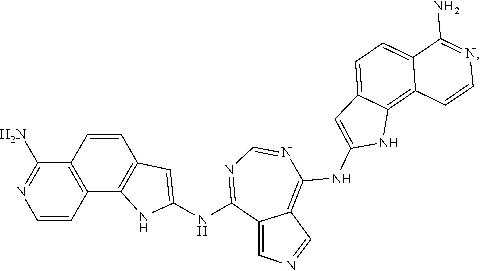 [SMILES: Nc1nccc2c1ccc1cc(Nc3ncnc(Nc4cc5ccc6c(N)nccc6c5[nH]4)c4cncc3-4)[nH]c12] | V-693 |
| 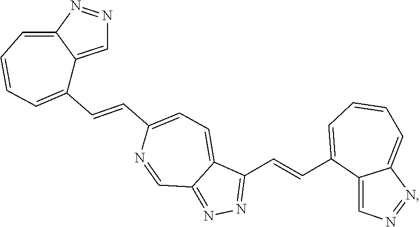 [SMILES: C(=C\c1ccccc2nncc1-2)/c1ccc2c(/C=C/c3ccccc4nncc3-4)nnc-2cn1] | V-694 |
| 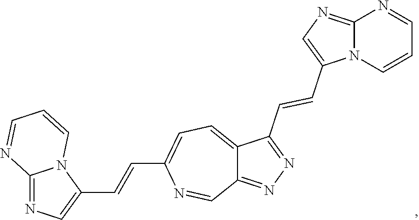 [SMILES: C(=C\c1cnc2ncccn12)/c1ccc2c(/C=C/c3cnc4ncccn34)nnc-2cn1] | V-695 |
| 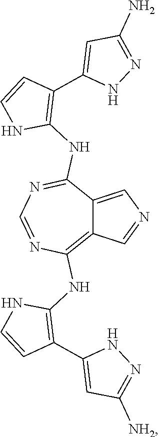 [SMILES: Nc1cc(-c2cc[nH]c2Nc2ncnc(Nc3[nH]ccc3-c3cc(N)n[nH]3)c3cncc2-3)[nH]n1] | V-696 |
| 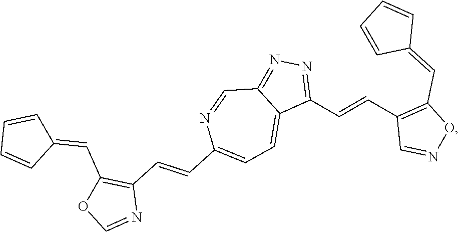 [SMILES: C1=CC(=Cc2oncc2/C=C/c2nnc3cnc(/C=C/c4ncoc4C=C4C=CC=C4)ccc2-3)C=C1] | V-697 |
| 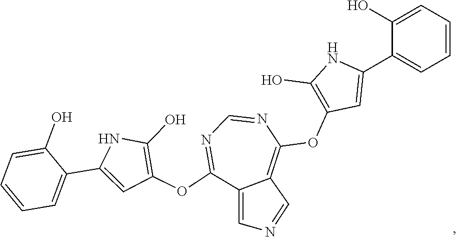 [SMILES: Oc1ccccc1-c1cc(Oc2ncnc(Oc3cc(-c4ccccc4O)[nH]c3O)c3cncc2-3)c(O)[nH]1] | V-698 |
| 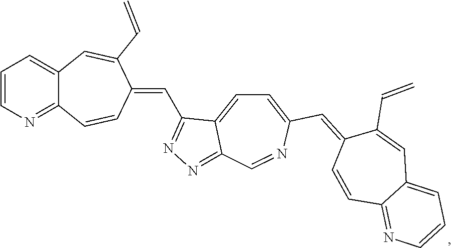 [SMILES: C=CC1=Cc2cccnc2C=C/C1=C\c1ccc2c(/C=C3\C=Cc4ncccc4C=C3C=C)nnc-2cn1] | V-699 |
| 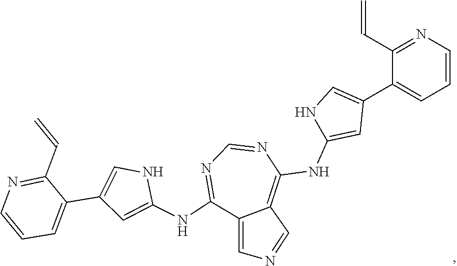 [SMILES: C=Cc1ncccc1-c1c[nH]c(Nc2ncnc(Nc3cc(-c4cccnc4C=C)c[nH]3)c3cncc2-3)c1] | V-700 |
| 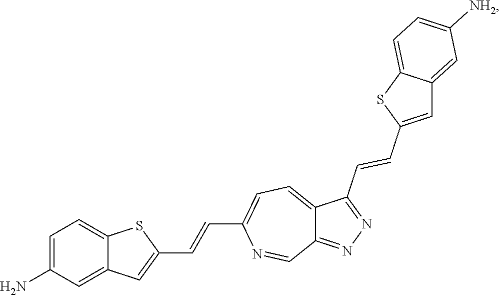 [SMILES: Nc1ccc2sc(/C=C/c3ccc4c(/C=C/c5cc6cc(N)ccc6s5)nnc-4cn3)cc2c1] | V-701 |
| 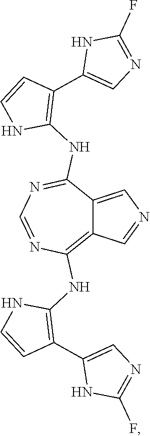 [SMILES: Fc1ncc(-c2cc[nH]c2Nc2ncnc(Nc3[nH]ccc3-c3cnc(F)[nH]3)c3cncc2-3)[nH]1] | V-702 |
| 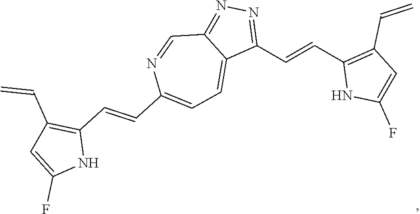 [SMILES: C=Cc1cc(F)[nH]c1/C=C/c1ccc2c(/C=C/c3[nH]c(F)cc3C=C)nnc-2cn1] | V-703 |
| 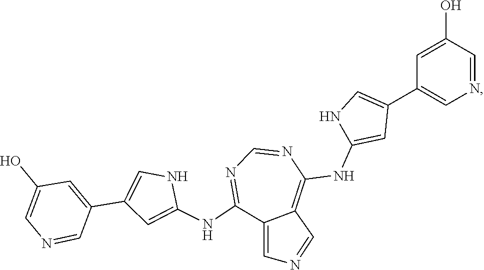 [SMILES: Oc1cncc(-c2c[nH]c(Nc3ncnc(Nc4cc(-c5cncc(O)c5)c[nH]4)c4cncc3-4)c2)c1] | V-704 |
| 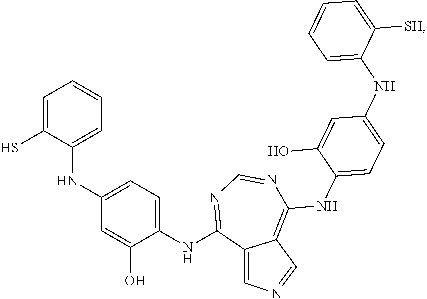 [SMILES: Oc1cc(Nc2ccccc2S)ccc1Nc1ncnc(Nc2ccc(Nc3ccccc3S)cc2O)c2cncc1-2] | V-705 |
| 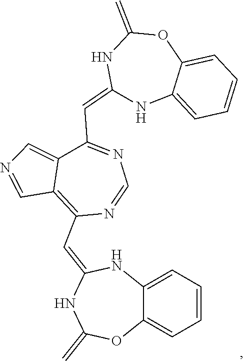 [SMILES: C=c1[nH]/c(=C/c2ncnc(/C=c3/[nH]c(=C)oc4ccccc4[nH]3)c3cncc2-3)[nH]c2ccccc2o1] | V-706 |
| 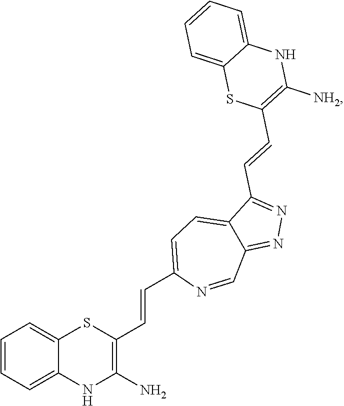 [SMILES: NC1=C(/C=C/c2ccc3c(/C=C/C4=C(N)Nc5ccccc5S4)nnc-3cn2)Sc2ccccc2N1] | V-707 |
| 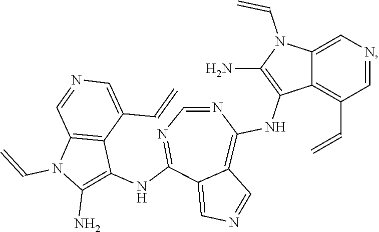 [SMILES: C=Cc1cncc2c1c(Nc1ncnc(Nc3c(N)n(C=C)c4cncc(C=C)c34)c3cncc1-3)c(N)n2C=C] | V-708 |
| 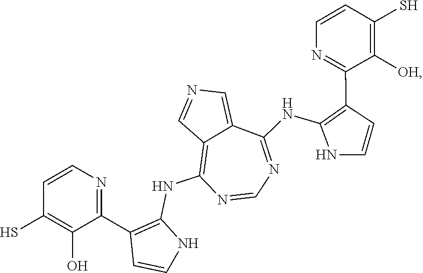 [SMILES: Oc1c(S)ccnc1-c1cc[nH]c1Nc1ncnc(Nc2[nH]ccc2-c2nccc(S)c2O)c2cncc1-2] | V-709 |
| 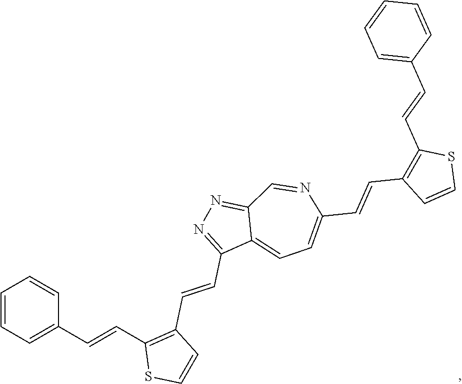 [SMILES: C(=C\c1sccc1/C=C/c1ccc2c(/C=C/c3ccsc3/C=C/c3ccccc3)nnc-2cn1)/c1ccccc1] | V-710 |
| 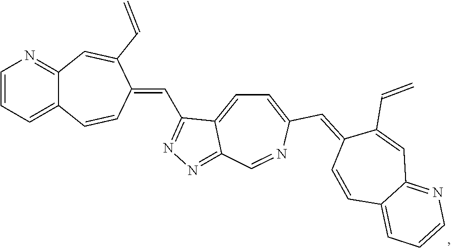 [SMILES: C=CC1=Cc2ncccc2C=C/C1=C\c1ccc2c(/C=C3\C=Cc4cccnc4C=C3C=C)nnc-2cn1] | V-711 |
| 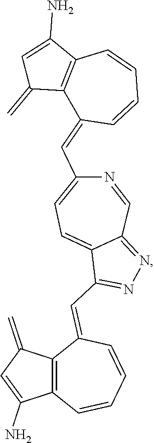 [SMILES: C=c1cc(N)c2cccc/c(=C\c3ccc4c(/C=c5\ccccc6c(N)cc(=C)c5=6)nnc-4cn3)c1=2] | V-712 |
| 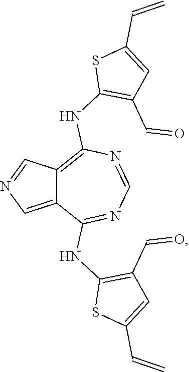 [SMILES: C=Cc1cc(C=O)c(Nc2ncnc(Nc3sc(C=C)cc3C=O)c3cncc2-3)s1] | V-713 |
| 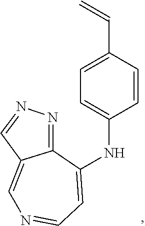 [SMILES: C=Cc1ccc(Nc2ccncc3cnnc2-3)cc1] | V-714 |
| 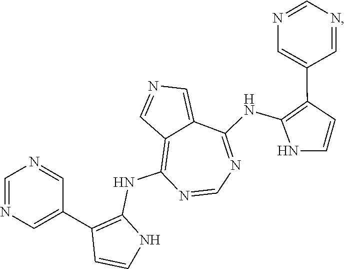 [SMILES: c1ncc(-c2cc[nH]c2Nc2ncnc(Nc3[nH]ccc3-c3cncnc3)c3cncc2-3)cn1] | V-715 |
| 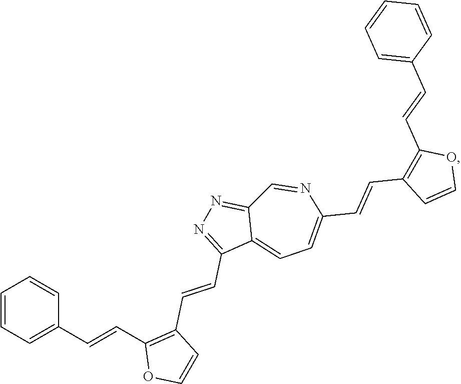 [SMILES: C(=C\c1occc1/C=C/c1ccc2c(/C=C/c3ccoc3/C=C/c3ccccc3)nnc-2cn1)/c1ccccc1] | V-716 |
| 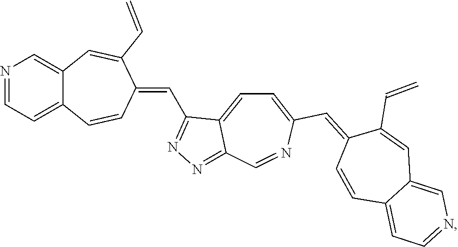 [SMILES: C=CC1=Cc2cnccc2C=C/C1=C\c1ccc2c(/C=C3\C=Cc4ccncc4C=C3C=C)nnc-2cn1] | V-717 |
| 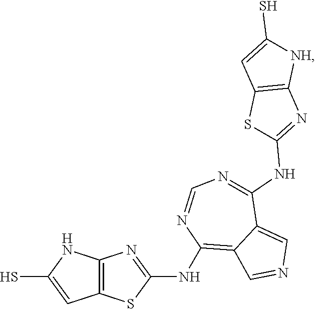 [SMILES: Sc1cc2sc(Nc3ncnc(Nc4nc5[nH]c(S)cc5s4)c4cncc3-4)nc2[nH]1] | V-718 |
| 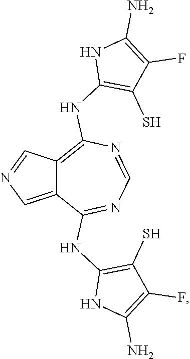 [SMILES: Nc1[nH]c(Nc2ncnc(Nc3[nH]c(N)c(F)c3S)c3cncc2-3)c(S)c1F] | V-719 |
| 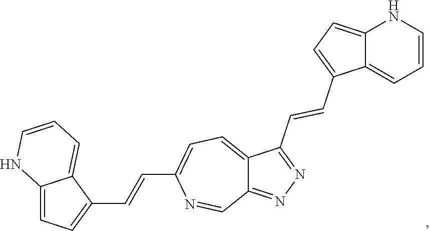 [SMILES: C(=C\c1ccc2[nH]cccc1-2)/c1ccc2c(/C=C/c3ccc4[nH]cccc3-4)nnc-2cn1] | V-720 |
| 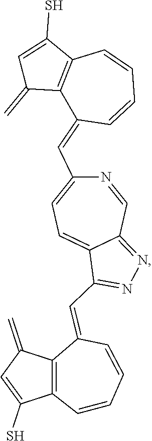 [SMILES: C=c1cc(S)c2cccc/c(=C\c3ccc4c(/C=c5\ccccc6c(S)cc(=C)c5=6)nnc-4cn3)c1=2] | V-721 |
| 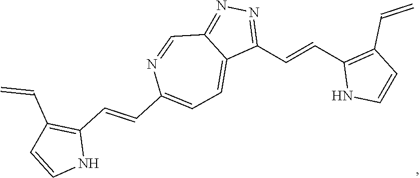 [SMILES: C=Cc1cc[nH]c1/C=C/c1ccc2c(/C=C/c3[nH]ccc3C=C)nnc-2cn1] | V-722 |
| 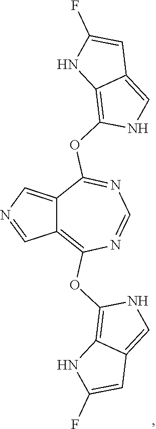 [SMILES: Fc1cc2c[nH]c(Oc3ncnc(Oc4[nH]cc5cc(F)[nH]c45)c4cncc3-4)c2[nH]1] | V-723 |
| 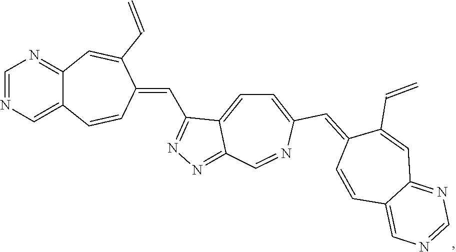 [SMILES: C=CC1=Cc2ncncc2C=C/C1=C\c1ccc2c(/C=C3\C=Cc4cncnc4C=C3C=C)nnc-2cn1] | V-724 |
| 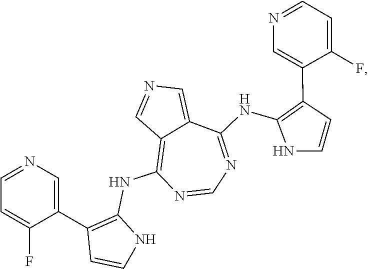 [SMILES: Fc1ccncc1-c1cc[nH]c1Nc1ncnc(Nc2[nH]ccc2-c2cnccc2F)c2cncc1-2] | V-725 |
| 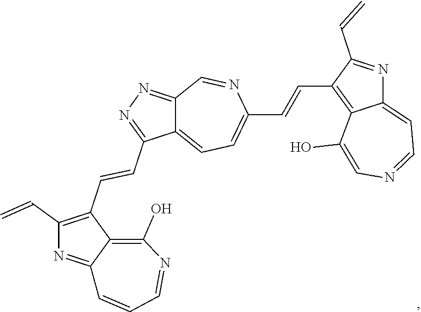 [SMILES: C=Cc1nc2ccncc(O)c-2c1/C=C/c1ccc2c(/C=C/c3c(C=C)nc4cccnc(O)c3-4)nnc-2cn1] | V-726 |
| 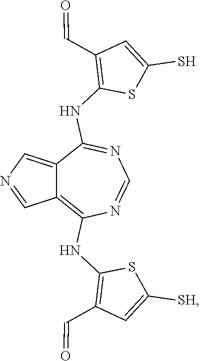 [SMILES: O=Cc1cc(S)sc1Nc1ncnc(Nc2sc(S)cc2C=O)c2cncc1-2] | V-727 |
| 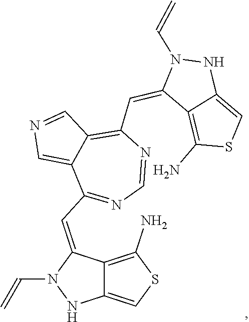 [SMILES: C=CN1Nc2csc(N)c2/C1=C\c1ncnc(/C=C2\c3c(csc3N)NN2C=C)c2cncc1-2] | V-728 |
| 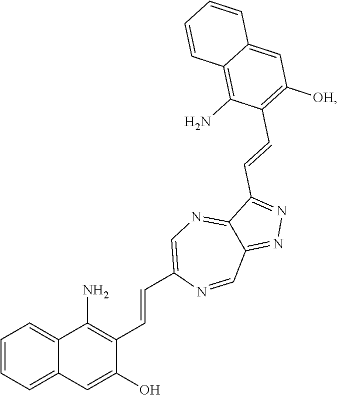 [SMILES: Nc1c(/C=C/c2cnc3c(/C=C/c4c(O)cc5ccccc5c4N)nnc-3cn2)c(O)cc2ccccc12] | V-729 |
| 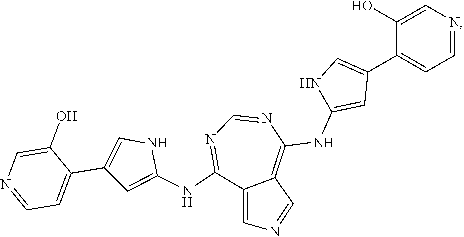 [SMILES: Oc1cnccc1-c1c[nH]c(Nc2ncnc(Nc3cc(-c4ccncc4O)c[nH]3)c3cncc2-3)c1] | V-730 |
| 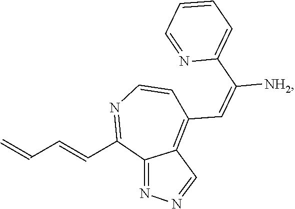 [SMILES: C=C/C=C/c1nccc(/C=C(/N)c2ccccn2)c2cnnc1-2] | V-731 |
| 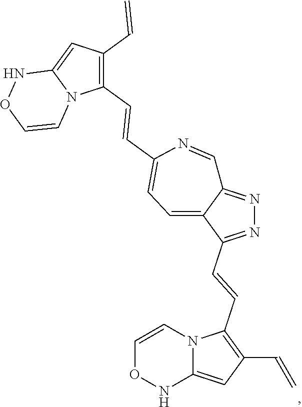 [SMILES: C=Cc1cc2n(c1/C=C/c1ccc3c(/C=C/c4c(C=C)cc5n4C=CON5)nnc-3cn1)C=CON2] | V-732 |
| 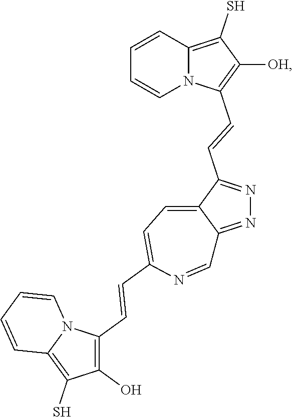 [SMILES: Oc1c(S)c2ccccn2c1/C=C/c1ccc2c(/C=C/c3c(O)c(S)c4ccccn34)nnc-2cn1] | V-733 |
| 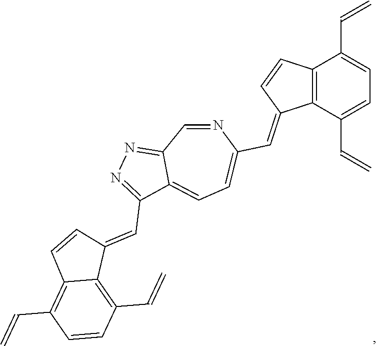 [SMILES: C=Cc1ccc(C=C)c2c1C=C/C2=C\c1ccc2c(/C=C3\C=Cc4c(C=C)ccc(C=C)c43)nnc-2cn1] | V-734 |
| 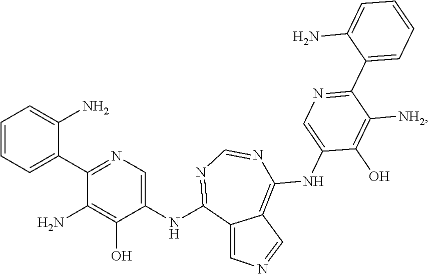 [SMILES: Nc1ccccc1-c1ncc(Nc2ncnc(Nc3cnc(-c4ccccc4N)c(N)c3O)c3cncc2-3)c(O)c1N] | V-735 |
| 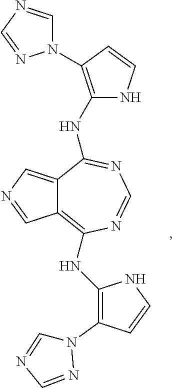 [SMILES: c1nc(Nc2[nH]ccc2-n2cncn2)c2cncc-2c(Nc2[nH]ccc2-n2cncn2)n1] | V-736 |
| 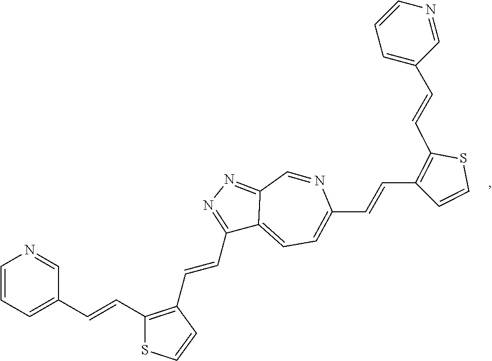 [SMILES: C(=C\c1sccc1/C=C/c1ccc2c(/C=C/c3ccsc3/C=C/c3cccnc3)nnc-2cn1)/c1cccnc1] | V-737 |
| 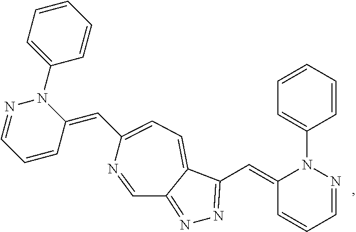 [SMILES: C1=C/C(=C\c2ccc3c(/C=C4\C=CC=NN4c4ccccc4)nnc-3cn2)N(c2ccccc2)N=C1] | V-738 |
| 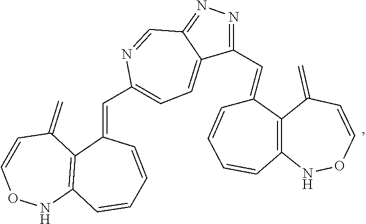 [SMILES: C=c1cco[nH]c2cccc/c(=C\c3ccc4c(/C=c5\ccccc6[nH]occc(=C)c5=6)nnc-4cn3)c1=2] | V-739 |
| 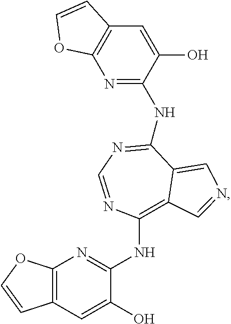 [SMILES: Oc1cc2ccoc2nc1Nc1ncnc(Nc2nc3occc3cc2O)c2cncc1-2] | V-740 |
| 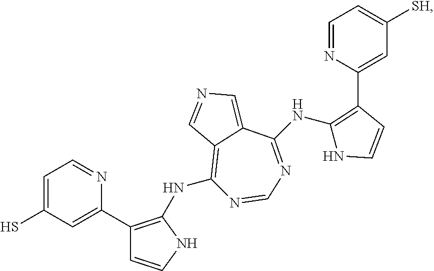 [SMILES: Sc1ccnc(-c2cc[nH]c2Nc2ncnc(Nc3[nH]ccc3-c3cc(S)ccn3)c3cncc2-3)c1] | V-741 |
| 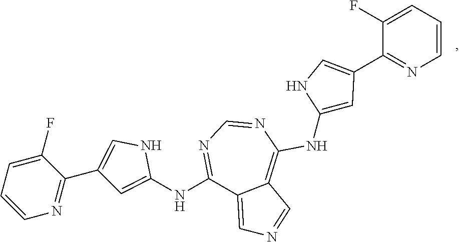 [SMILES: Fc1cccnc1-c1c[nH]c(Nc2ncnc(Nc3cc(-c4ncccc4F)c[nH]3)c3cncc2-3)c1] | V-742 |
| 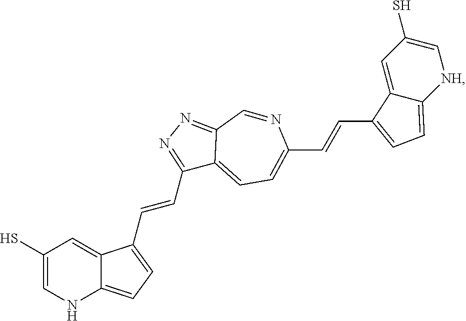 [SMILES: Sc1c[nH]c2ccc(/C=C/c3ccc4c(/C=C/c5ccc6[nH]cc(S)cc5-6)nnc-4cn3)c-2c1] | V-743 |
| 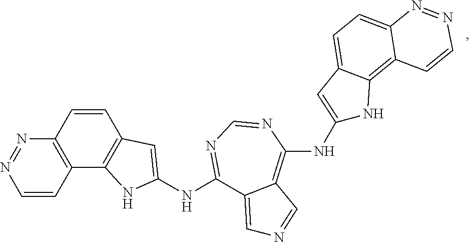 [SMILES: c1cc2c(ccc3cc(Nc4ncnc(Nc5cc6ccc7nnccc7c6[nH]5)c5cncc4-5)[nH]c32)nn1] | V-744 |
| 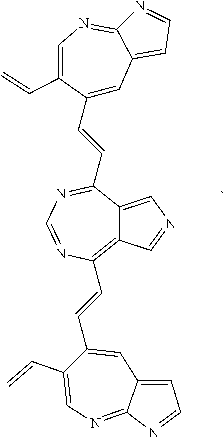 [SMILES: C=Cc1cnc2nccc-2cc1/C=C/c1ncnc(/C=C/c2cc3ccnc-3ncc2C=C)c2cncc1-2] | V-745 |
| 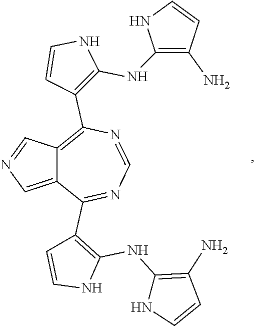 [SMILES: Nc1cc[nH]c1Nc1[nH]ccc1-c1ncnc(-c2cc[nH]c2Nc2[nH]ccc2N)c2cncc1-2] | V-746 |
| 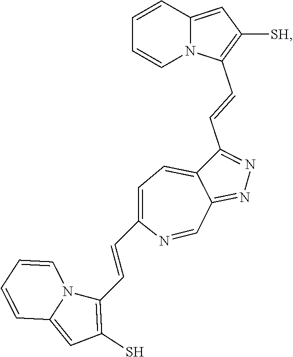 [SMILES: Sc1cc2ccccn2c1/C=C/c1ccc2c(/C=C/c3c(S)cc4ccccn34)nnc-2cn1] | V-747 |
| 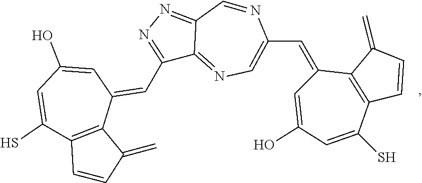 [SMILES: C=c1ccc2c(S)cc(O)c/c(=C\c3cnc4c(/C=c5\cc(O)cc(S)c6ccc(=C)c5=6)nnc-4cn3)c1=2] | V-748 |
| 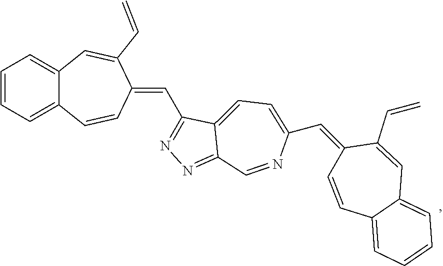 [SMILES: C=CC1=Cc2ccccc2C=C/C1=C\c1ccc2c(/C=C3\C=Cc4ccccc4C=C3C=C)nnc-2cn1] | V-749 |
| 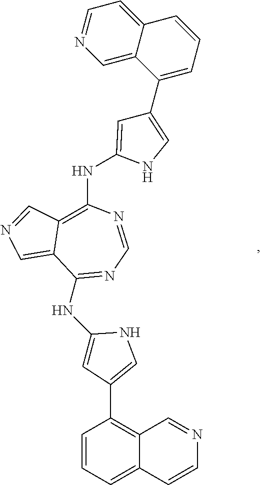 [SMILES: c1cc(-c2c[nH]c(Nc3ncnc(Nc4cc(-c5cccc6ccncc56)c[nH]4)c4cncc3-4)c2)c2cnccc2c1] | V-750 |
| 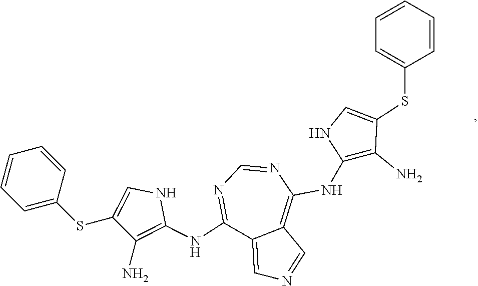 [SMILES: Nc1c(Sc2ccccc2)c[nH]c1Nc1ncnc(Nc2[nH]cc(Sc3ccccc3)c2N)c2cncc1-2] | V-751 |
| 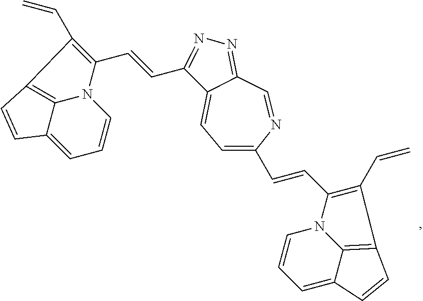 [SMILES: C=Cc1c(/C=C/c2ccc3c(/C=C/c4c(C=C)c5ccc6cccn4c65)nnc-3cn2)n2cccc3ccc1c32] | V-752 |
| 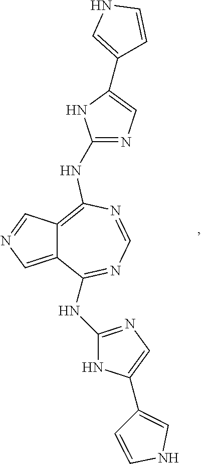 [SMILES: c1nc(Nc2ncc(-c3cc[nH]c3)[nH]2)c2cncc-2c(Nc2ncc(-c3cc[nH]c3)[nH]2)n1] | V-753 |
| 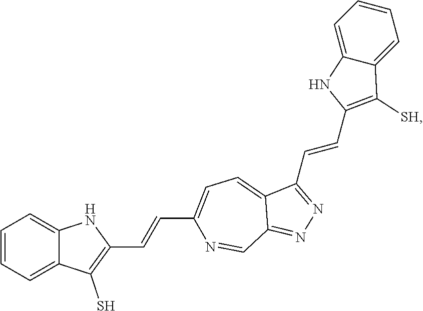 [SMILES: Sc1c(/C=C/c2ccc3c(/C=C/c4[nH]c5ccccc5c4S)nnc-3cn2)[nH]c2ccccc12] | V-754 |
| 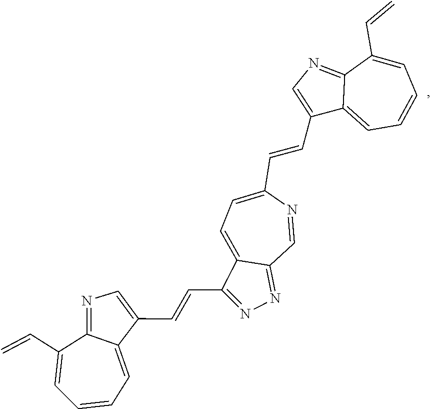 [SMILES: C=Cc1ccccc2c(/C=C/c3ccc4c(/C=C/c5cnc6c(C=C)ccccc5-6)nnc-4cn3)cnc1-2] | V-755 |
| 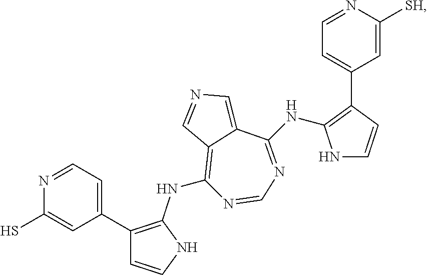 [SMILES: Sc1cc(-c2cc[nH]c2Nc2ncnc(Nc3[nH]ccc3-c3ccnc(S)c3)c3cncc2-3)ccn1] | V-756 |
| 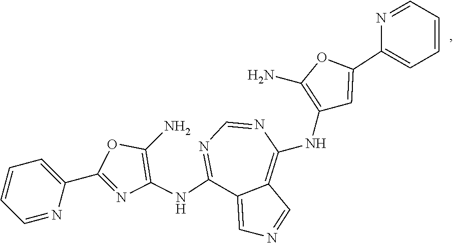 [SMILES: Nc1oc(-c2ccccn2)cc1Nc1ncnc(Nc2nc(-c3ccccn3)oc2N)c2cncc1-2] | V-757 |
| 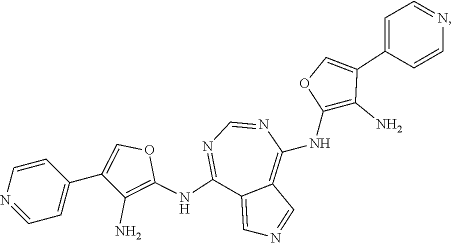 [SMILES: Nc1c(-c2ccncc2)coc1Nc1ncnc(Nc2occ(-c3ccncc3)c2N)c2cncc1-2] | V-758 |
| 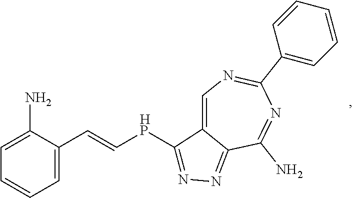 | V-759 |
| 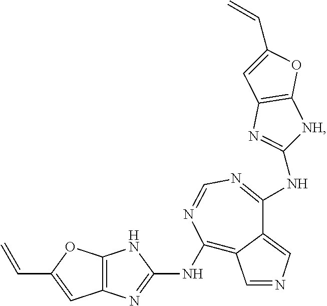 [SMILES: C=Cc1cc2nc(Nc3ncnc(Nc4nc5cc(C=C)oc5[nH]4)c4cncc3-4)[nH]c2o1] | V-760 |
| 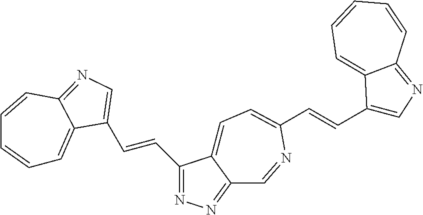 [SMILES: C(=C\c1cnc2cccccc1-2)/c1ccc2c(/C=C/c3cnc4cccccc3-4)nnc-2cn1] | V-761 |
| 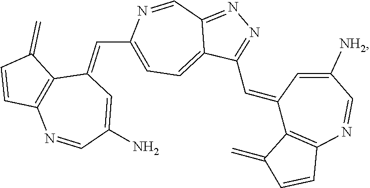 [SMILES: C=c1ccc2ncc(N)c/c(=C\c3ccc4c(/C=c5\cc(N)cnc6ccc(=C)c5=6)nnc-4cn3)c1=2] | V-762 |
| 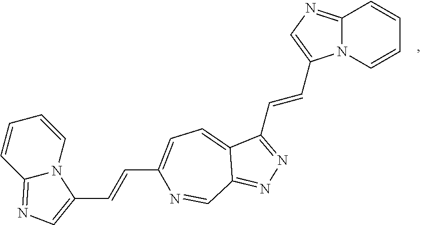 [SMILES: C(=C\c1cnc2ccccn12)/c1ccc2c(/C=C/c3cnc4ccccn34)nnc-2cn1] | V-763 |
| 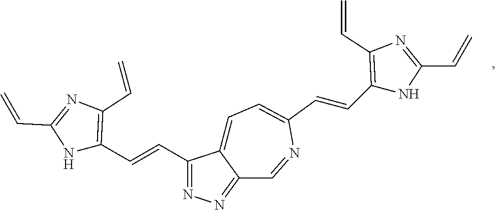 [SMILES: C=Cc1nc(C=C)c(/C=C/c2ccc3c(/C=C/c4[nH]c(C=C)nc4C=C)nnc-3cn2)[nH]1] | V-764 |
| 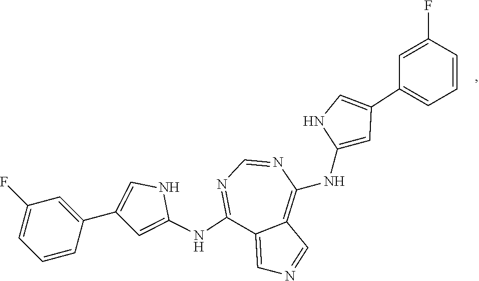 [SMILES: Fc1cccc(-c2c[nH]c(Nc3ncnc(Nc4cc(-c5cccc(F)c5)c[nH]4)c4cncc3-4)c2)c1] | V-765 |
| 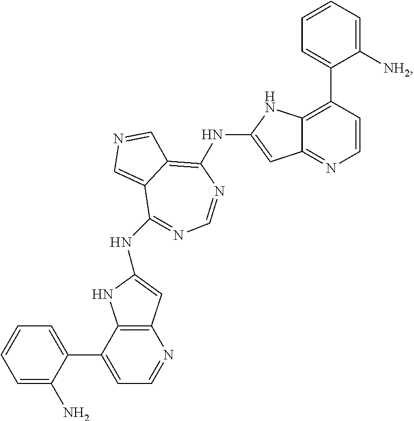 [SMILES: Nc1ccccc1-c1ccnc2cc(Nc3ncnc(Nc4cc5nccc(-c6ccccc6N)c5[nH]4)c4cncc3-4)[nH]c12] | V-766 |
| 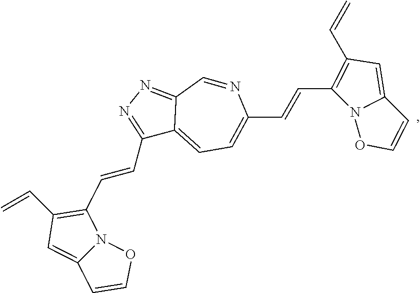 [SMILES: C=Cc1cc2ccon2c1/C=C/c1ccc2c(/C=C/c3c(C=C)cc4ccon34)nnc-2cn1] | V-767 |
| 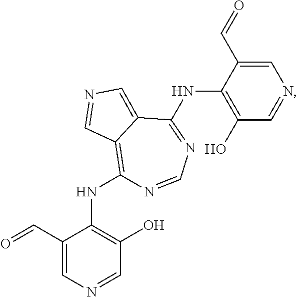 [SMILES: O=Cc1cncc(O)c1Nc1ncnc(Nc2c(O)cncc2C=O)c2cncc1-2] | V-768 |
| 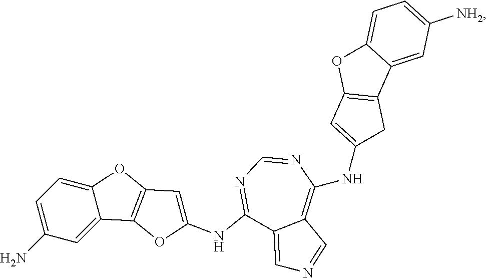 [SMILES: Nc1ccc2oc3c(c2c1)CC(Nc1ncnc(Nc2cc4oc5ccc(N)cc5c4o2)c2cncc1-2)=C3] | V-769 |
| 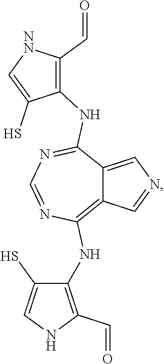 | V-770 |
| 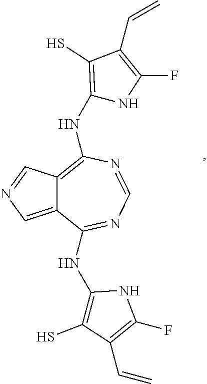 [SMILES: C=Cc1c(F)[nH]c(Nc2ncnc(Nc3[nH]c(F)c(C=C)c3S)c3cncc2-3)c1S] | V-771 |
| 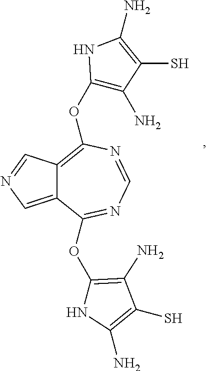 [SMILES: Nc1[nH]c(Oc2ncnc(Oc3[nH]c(N)c(S)c3N)c3cncc2-3)c(N)c1S] | V-772 |
| 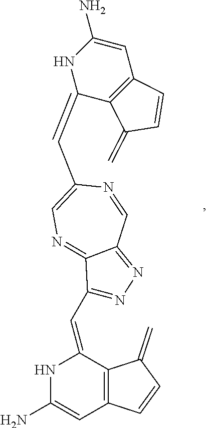 [SMILES: C=c1ccc2cc(N)[nH]/c(=C/c3cnc4c(/C=c5/[nH]c(N)cc6ccc(=C)c5=6)nnc-4cn3)c1=2] | V-773 |
| 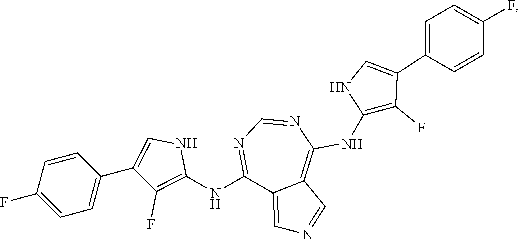 [SMILES: Fc1ccc(-c2c[nH]c(Nc3ncnc(Nc4[nH]cc(-c5ccc(F)cc5)c4F)c4cncc3-4)c2F)cc1] | V-774 |
| 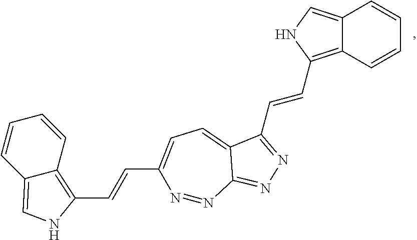 [SMILES: C(=C\c1[nH]cc2ccccc12)/c1ccc2c(/C=C/c3[nH]cc4ccccc34)nnc-2nn1] | V-775 |
| 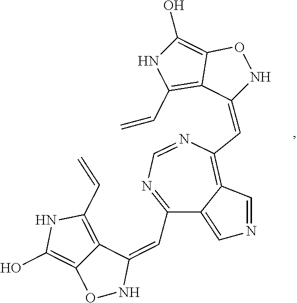 [SMILES: C=Cc1[nH]c(O)c2c1/C(=C\c1ncnc(/C=C3/NOc4c(O)[nH]c(C=C)c43)c3cncc1-3)NO2] | V-776 |
| 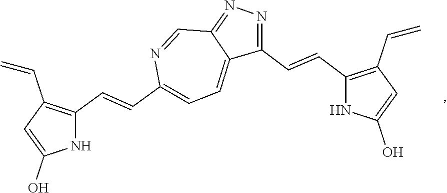 [SMILES: C=Cc1cc(O)[nH]c1/C=C/c1ccc2c(/C=C/c3[nH]c(O)cc3C=C)nnc-2cn1] | V-777 |
| 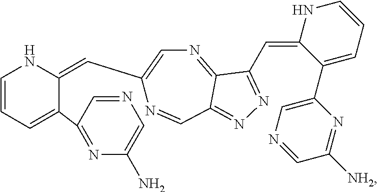 [SMILES: Nc1cncc(C2=CC=CN/C2=C/c2cnc3c(/C=C4/NC=CC=C4c4cncc(N)n4)nnc-3cn2)n1] | V-778 |
| 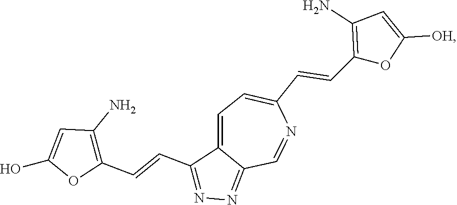 [SMILES: Nc1cc(O)oc1/C=C/c1ccc2c(/C=C/c3oc(O)cc3N)nnc-2cn1] | V-779 |
| 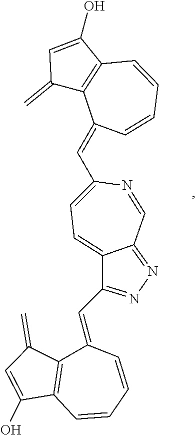 [SMILES: C=c1cc(O)c2cccc/c(=C\c3ccc4c(/C=c5\ccccc6c(O)cc(=C)c5=6)nnc-4cn3)c1=2] | V-780 |
| 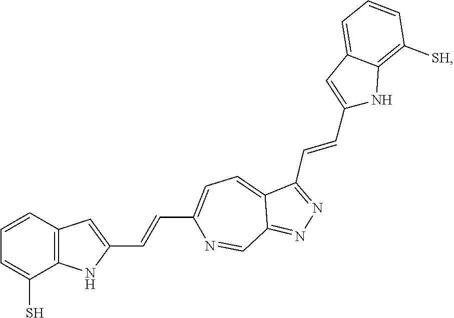 [SMILES: Sc1cccc2cc(/C=C/c3ccc4c(/C=C/c5cc6cccc(S)c6[nH]5)nnc-4cn3)[nH]c12] | V-781 |
| 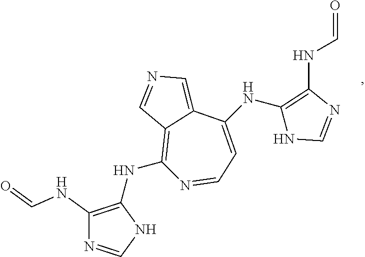 [SMILES: O=CNc1nc[nH]c1Nc1ccnc(Nc2[nH]cnc2NC=O)c2cncc1-2] | V-782 |
| 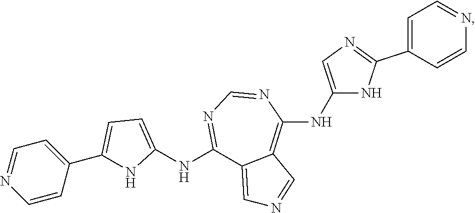 [SMILES: c1cc(-c2ccc(Nc3ncnc(Nc4cnc(-c5ccncc5)[nH]4)c4cncc3-4)[nH]2)ccn1] | V-783 |
| 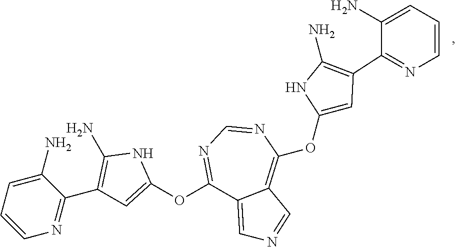 [SMILES: Nc1cccnc1-c1cc(Oc2ncnc(Oc3cc(-c4ncccc4N)c(N)[nH]3)c3cncc2-3)[nH]c1N] | V-784 |
| 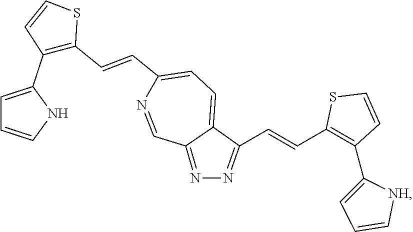 [SMILES: C(=C\c1sccc1-c1ccc[nH]1)/c1ccc2c(/C=C/c3sccc3-c3ccc[nH]3)nnc-2cn1] | V-785 |
| 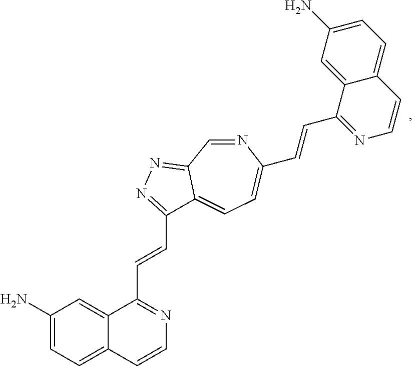 [SMILES: Nc1ccc2ccnc(/C=C/c3ccc4c(/C=C/c5nccc6ccc(N)cc56)nnc-4cn3)c2c1] | V-786 |
| 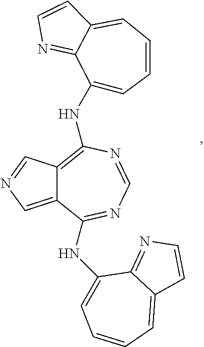 [SMILES: c1ccc(Nc2ncnc(Nc3ccccc4ccnc3-4)c3cncc2-3)c2nccc-2c1 ; ","] | V-787 |
| 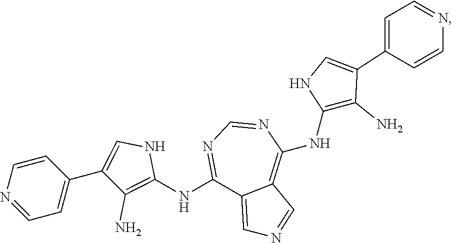 [SMILES: Nc1c(-c2ccncc2)c[nH]c1Nc1ncnc(Nc2[nH]cc(-c3ccncc3)c2N)c2cncc1-2] | V-788 |
| 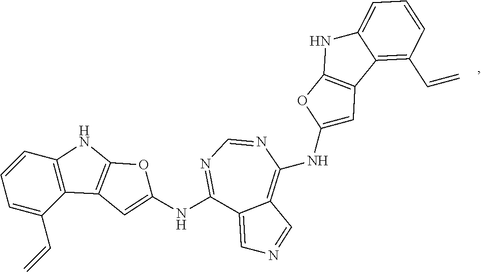 [SMILES: C=Cc1cccc2[nH]c3oc(Nc4ncnc(Nc5cc6c([nH]c7cccc(C=C)c76)o5)c5cncc4-5)cc3c12] | V-789 |
| 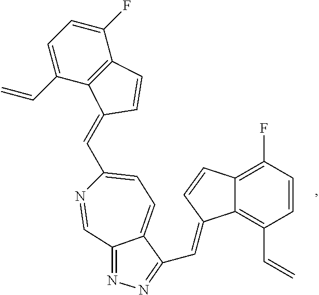 [SMILES: C=Cc1ccc(F)c2c1/C(=C/c1ccc3c(/C=C4\C=Cc5c(F)ccc(C=C)c54)nnc-3cn1)C=C2] | V-790 |
| 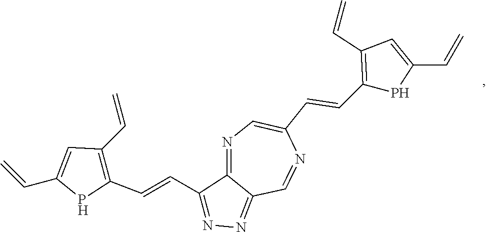 | V-791 |
| 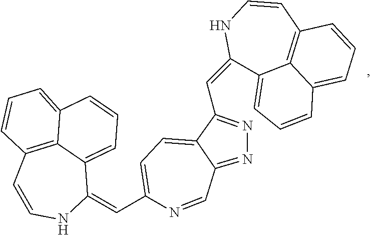 [SMILES: C(/c1ccc2c(/C=c3/[nH]ccc4cccc5cccc3c54)nnc-2cn1)=c1\[nH]ccc2cccc3cccc1c32] | V-792 |
| 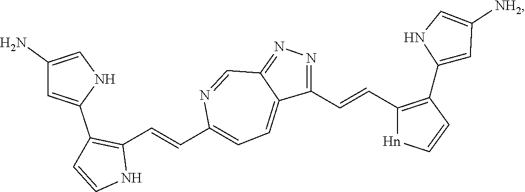 | V-793 |
| 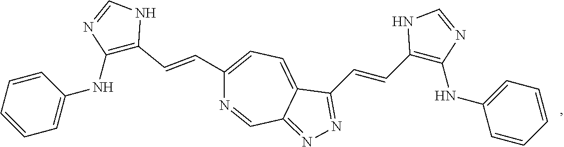 [SMILES: C(=C\c1[nH]cnc1Nc1ccccc1)/c1ccc2c(/C=C/c3[nH]cnc3Nc3ccccc3)nnc-2cn1] | V-794 |
| 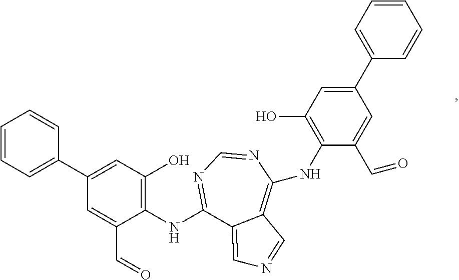 [SMILES: O=Cc1cc(-c2ccccc2)cc(O)c1Nc1ncnc(Nc2c(O)cc(-c3ccccc3)cc2C=O)c2cncc1-2] | V-795 |
| 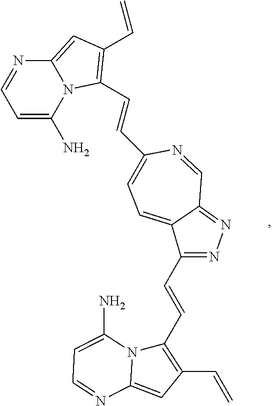 [SMILES: C=Cc1cc2nccc(N)n2c1/C=C/c1ccc2c(/C=C/c3c(C=C)cc4nccc(N)n34)nnc-2cn1] | V-796 |
| 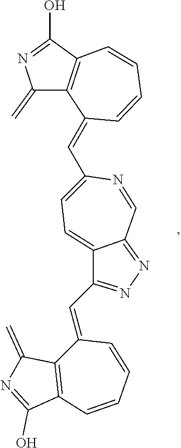 [SMILES: C=c1nc(O)c2cccc/c(=C\c3ccc4c(/C=c5\ccccc6c(O)nc(=C)c5=6)nnc-4cn3)c1=2] | V-797 |
| 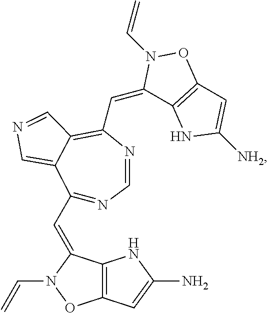 [SMILES: C=CN1Oc2cc(N)[nH]c2/C1=C\c1ncnc(/C=C2\c3[nH]c(N)cc3ON2C=C)c2cncc1-2] | V-798 |
| 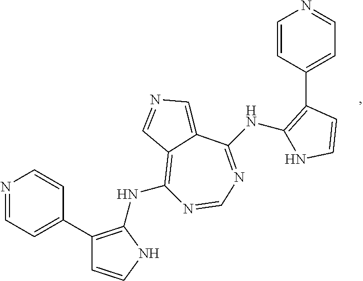 [SMILES: c1cc(-c2cc[nH]c2Nc2ncnc(Nc3[nH]ccc3-c3ccncc3)c3cncc2-3)ccn1] | V-799 |
| 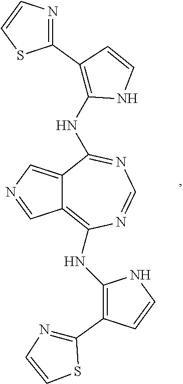 [SMILES: c1csc(-c2cc[nH]c2Nc2ncnc(Nc3[nH]ccc3-c3nccs3)c3cncc2-3)n1] | V-800 |
| 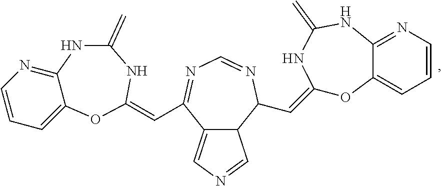 [SMILES: C=c1[nH]/c(=C\C2=NC=NC(/C=c3\[nH]c(=C)[nH]c4ncccc4o3)C3C=NC=C23)oc2cccnc2[nH]1] | V-801 |
| 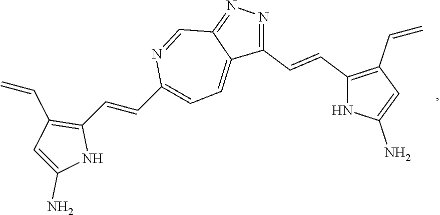 [SMILES: C=Cc1cc(N)[nH]c1/C=C/c1ccc2c(/C=C/c3[nH]c(N)cc3C=C)nnc-2cn1] | V-802 |
| 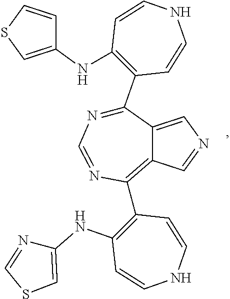 [SMILES: C1=CC(Nc2ccsc2)=C(c2ncnc(C3=C(Nc4cscn4)C=CNC=C3)c3cncc2-3)C=CN1] | V-803 |
| 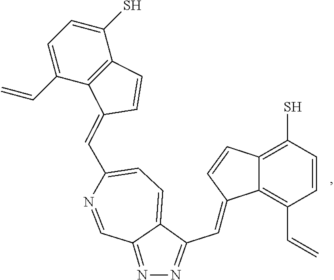 [SMILES: C=Cc1ccc(S)c2c1/C(=C/c1ccc3c(/C=C4\C=Cc5c(S)ccc(C=C)c54)nnc-3cn1)C=C2] | V-804 |
| 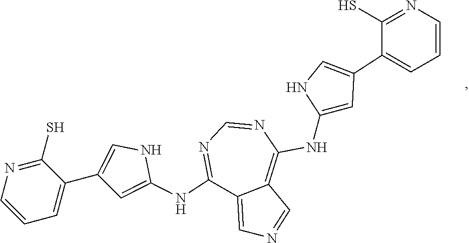 [SMILES: Sc1ncccc1-c1c[nH]c(Nc2ncnc(Nc3cc(-c4cccnc4S)c[nH]3)c3cncc2-3)c1] | V-805 |
| 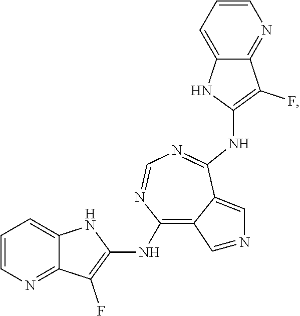 [SMILES: Fc1c(Nc2ncnc(Nc3[nH]c4cccnc4c3F)c3cncc2-3)[nH]c2cccnc12] | V-806 |
| 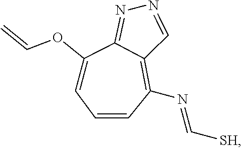 [SMILES: C=COc1cccc(/N=C/S)c2cnnc1-2] | V-807 |
| 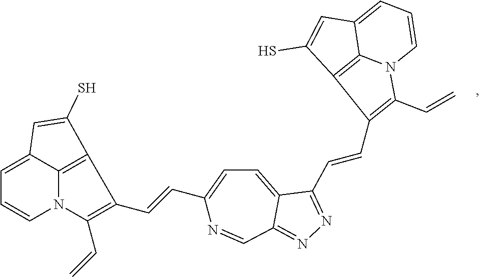 [SMILES: C=Cc1c(/C=C/c2ccc3c(/C=C/c4c(C=C)n5cccc6cc(S)c4c65)nnc-3cn2)c2c(S)cc3cccn1c32] | V-808 |
| 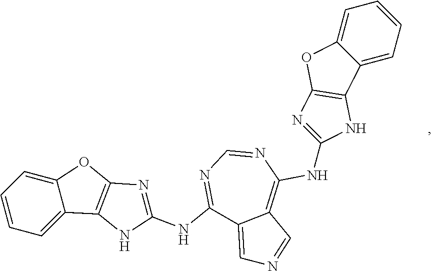 [SMILES: c1ccc2c(c1)oc1nc(Nc3ncnc(Nc4nc5oc6ccccc6c5[nH]4)c4cncc3-4)[nH]c12] | V-809 |
| 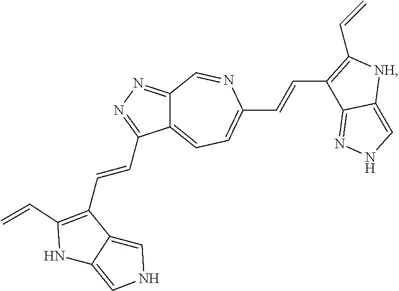 [SMILES: C=Cc1[nH]c2c[nH]cc2c1/C=C/c1nnc2cnc(/C=C/c3c(C=C)[nH]c4c[nH]nc34)ccc1-2] | V-810 |
| 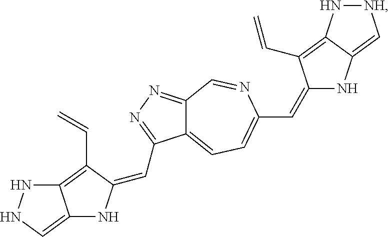 [SMILES: C=Cc1c2c([nH]/c1=C/c1ccc3c(/C=c4/[nH]c5c(c4C=C)NNC=5)nnc-3cn1)=CNN2] | V-811 |
| 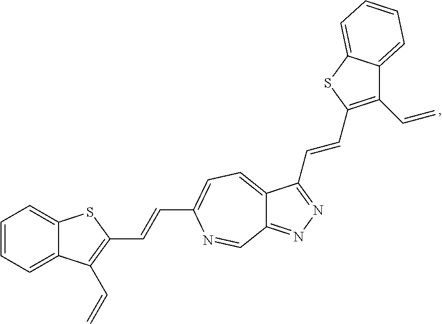 [SMILES: C=Cc1c(/C=C/c2ccc3c(/C=C/c4sc5ccccc5c4C=C)nnc-3cn2)sc2ccccc12] | V-812 |
| 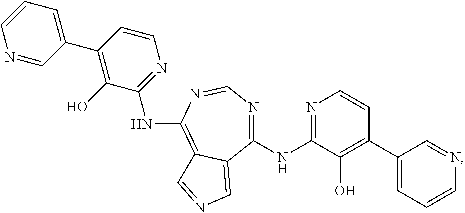 [SMILES: Oc1c(-c2cccnc2)ccnc1Nc1ncnc(Nc2nccc(-c3cccnc3)c2O)c2cncc1-2] | V-813 |
| 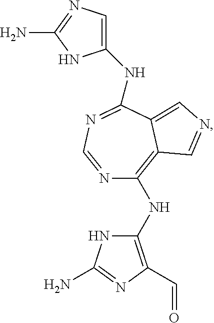 [SMILES: Nc1ncc(Nc2ncnc(Nc3[nH]c(N)nc3C=O)c3cncc2-3)[nH]1] | V-814 |
| 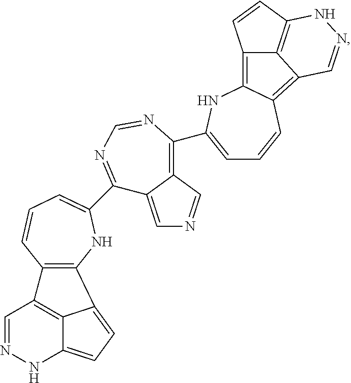 [SMILES: c1cc(-c2ncnc(-c3cccc4c5cn[nH]c6ccc(c4[nH]3)c65)c3cncc2-3)[nH]c2c(c1)c1cn[nH]c3ccc2c31] | V-815 |
| 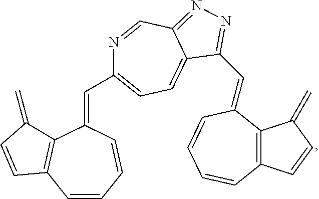 [SMILES: C=c1ccc2cccc/c(=C\c3ccc4c(/C=c5\ccccc6ccc(=C)c5=6)nnc-4cn3)c1=2] | V-816 |
| 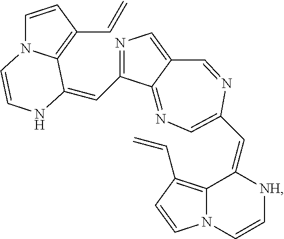 [SMILES: C=Cc1ccn2c1/C(=C\c1cnc3c(/C=C4/NC=Cn5ccc(C=C)c54)ncc-3cn1)NC=C2] | V-817 |
| 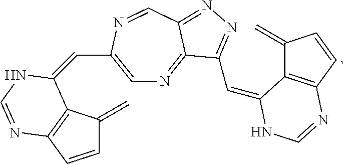 [SMILES: C=c1ccc2nc[nH]/c(=C/c3cnc4c(/C=c5/[nH]cnc6ccc(=C)c5=6)nnc-4cn3)c1=2] | V-818 |
| 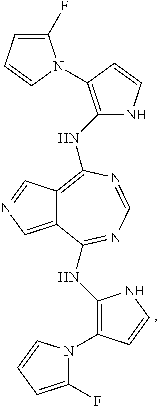 [SMILES: Fc1cccn1-c1cc[nH]c1Nc1ncnc(Nc2[nH]ccc2-n2cccc2F)c2cncc1-2] | V-819 |
| 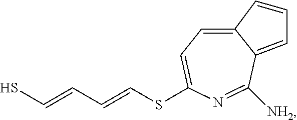 [SMILES: Nc1nc(S/C=C/C=C/S)ccc2cccc1-2] | V-820 |
| 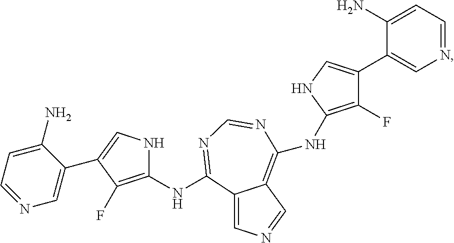 [SMILES: Nc1ccncc1-c1c[nH]c(Nc2ncnc(Nc3[nH]cc(-c4cnccc4N)c3F)c3cncc2-3)c1F] | V-821 |
| 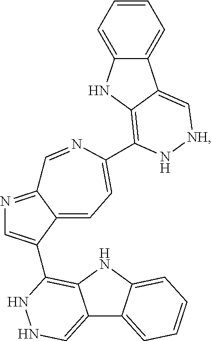 [SMILES: C1=c2c([nH]c3ccccc23)=C(c2ccc3c(C4=c5[nH]c6ccccc6c5=CNN4)cnc-3cn2)NN1] | V-822 |
| 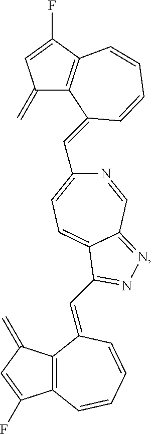 [SMILES: C=c1cc(F)c2cccc/c(=C\c3ccc4c(/C=c5\ccccc6c(F)cc(=C)c5=6)nnc-4cn3)c1=2] | V-823 |
| 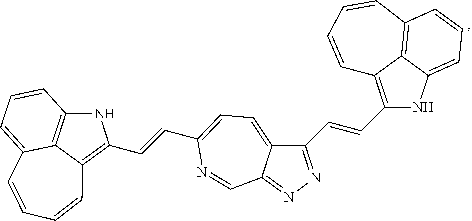 [SMILES: C1=Cc2cccc3[nH]c(/C=C/c4ccc5c(/C=C/c6[nH]c7cccc8c7c6C=CC=C8)nnc-5cn4)c(c23)C=C1] | V-824 |
| 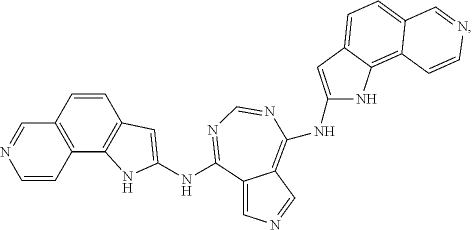 [SMILES: c1cc2c(ccc3cc(Nc4ncnc(Nc5cc6ccc7cnccc7c6[nH]5)c5cncc4-5)[nH]c32)cn1] | V-825 |
| 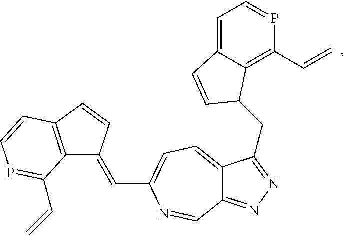 [SMILES: C=Cc1pccc2c1/C(=C/c1ccc3c(CC4C=Cc5ccpc(C=C)c54)nnc-3cn1)C=C2] | V-826 |
| 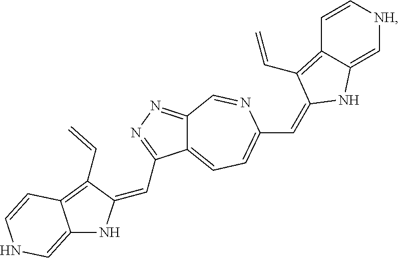 [SMILES: C=Cc1c2c([nH]/c1=C/c1ccc3c(/C=c4/[nH]c5c(c4C=C)C=CNC=5)nnc-3cn1)=CNC=C2] | V-827 |
| 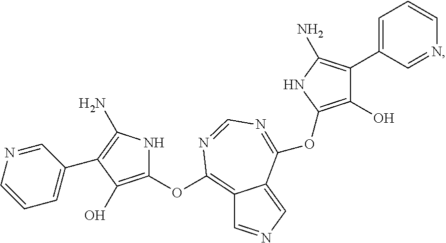 [SMILES: Nc1[nH]c(Oc2ncnc(Oc3[nH]c(N)c(-c4cccnc4)c3O)c3cncc2-3)c(O)c1-c1cccnc1] | V-828 |
| 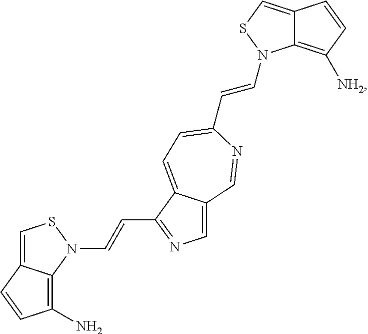 [SMILES: Nc1ccc2csn(/C=C/c3ccc4c(/C=C/n5scc6ccc(N)c5-6)ncc-4cn3)c1-2] | V-829 |
| 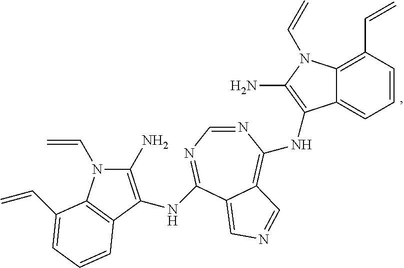 [SMILES: C=Cc1cccc2c(Nc3ncnc(Nc4c(N)n(C=C)c5c(C=C)cccc45)c4cncc3-4)c(N)n(C=C)c12] | V-830 |
| 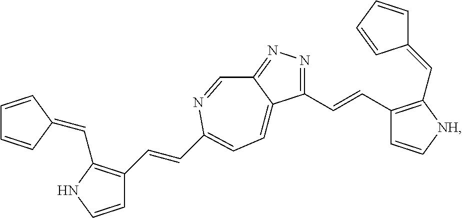 [SMILES: C1=CC(=Cc2[nH]ccc2/C=C/c2ccc3c(/C=C/c4cc[nH]c4C=C4C=CC=C4)nnc-3cn2)C=C1] | V-831 |
| 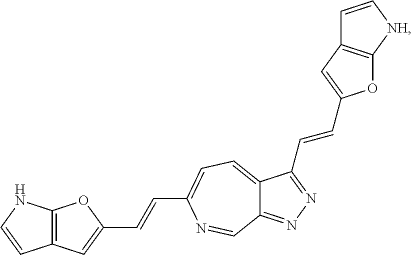 [SMILES: C(=C\c1cc2cc[nH]c2o1)/c1ccc2c(/C=C/c3cc4cc[nH]c4o3)nnc-2cn1] | V-832 |
| 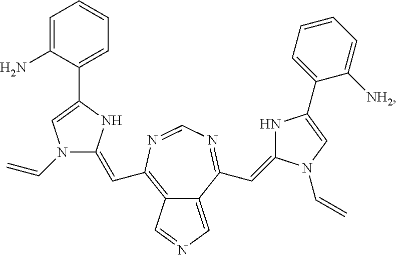 [SMILES: C=CN1C=C(c2ccccc2N)N/C1=C\c1ncnc(/C=C2\NC(c3ccccc3N)=CN2C=C)c2cncc1-2] | V-833 |
| 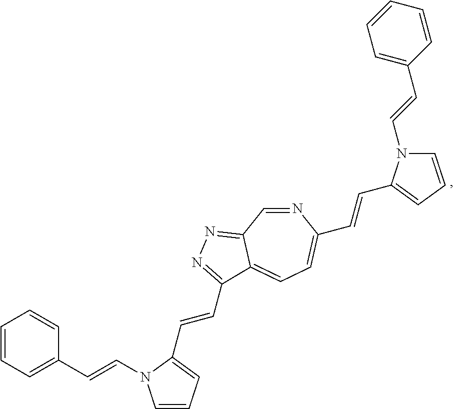 [SMILES: C(=C\n1cccc1/C=C/c1ccc2c(/C=C/c3cccn3/C=C/c3ccccc3)nnc-2cn1)/c1ccccc1] | V-834 |
| 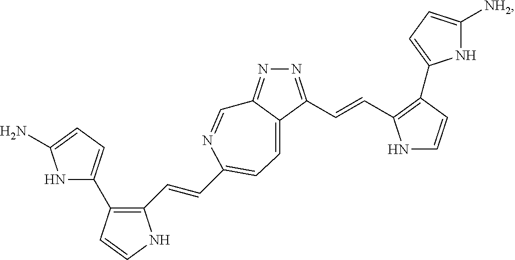 [SMILES: Nc1ccc(-c2cc[nH]c2/C=C/c2ccc3c(/C=C/c4[nH]ccc4-c4ccc(N)[nH]4)nnc-3cn2)[nH]1] | V-835 |
| 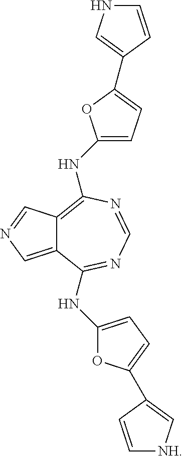 [SMILES: c1nc(Nc2ccc(-c3cc[nH]c3)o2)c2cncc-2c(Nc2ccc(-c3cc[nH]c3)o2)n1] | V-836 |
| 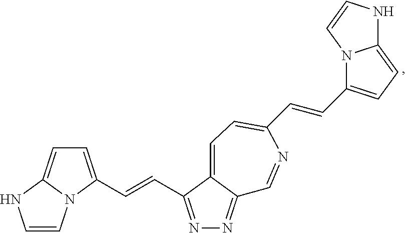 [SMILES: C(=C\c1ccc2[nH]ccn12)/c1ccc2c(/C=C/c3ccc4[nH]ccn34)nnc-2cn1] | V-837 |
| 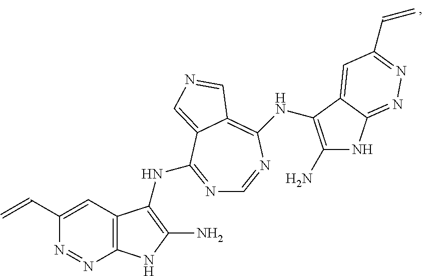 [SMILES: C=Cc1cc2c(Nc3ncnc(Nc4c(N)[nH]c5nnc(C=C)cc45)c4cncc3-4)c(N)[nH]c2nn1] | V-838 |
| 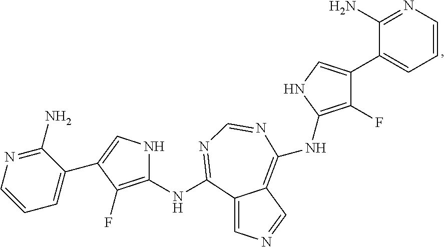 [SMILES: Nc1ncccc1-c1c[nH]c(Nc2ncnc(Nc3[nH]cc(-c4cccnc4N)c3F)c3cncc2-3)c1F] | V-839 |
| 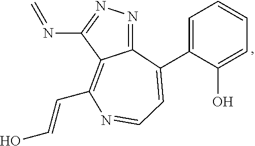 [SMILES: C=Nc1nnc2c(-c3ccccc3O)ccnc(/C=C/O)c1-2] | V-840 |
| 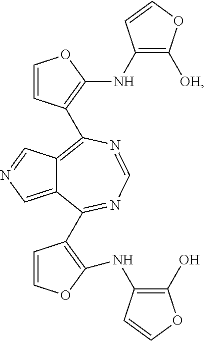 [SMILES: Oc1occc1Nc1occc1-c1ncnc(-c2ccoc2Nc2ccoc2O)c2cncc1-2] | V-841 |
| 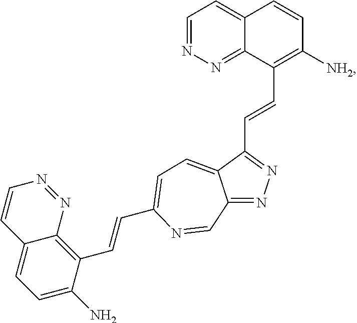 [SMILES: Nc1ccc2ccnnc2c1/C=C/c1ccc2c(/C=C/c3c(N)ccc4ccnnc34)nnc-2cn1] | V-842 |
| 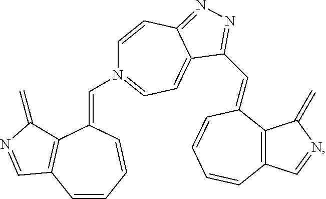 | V-843 |
| 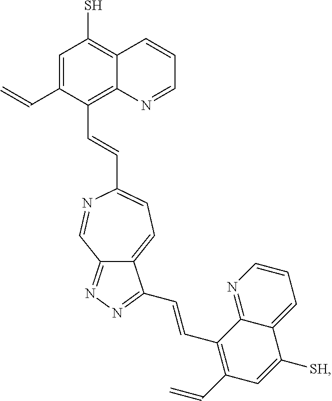 [SMILES: C=Cc1cc(S)c2cccnc2c1/C=C/c1ccc2c(/C=C/c3c(C=C)cc(S)c4cccnc34)nnc-2cn1] | V-844 |
| 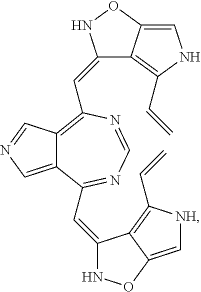 [SMILES: C=Cc1[nH]cc2c1/C(=C\c1ncnc(/C=C3/NOc4c[nH]c(C=C)c43)c3cncc1-3)NO2] | V-845 |
| 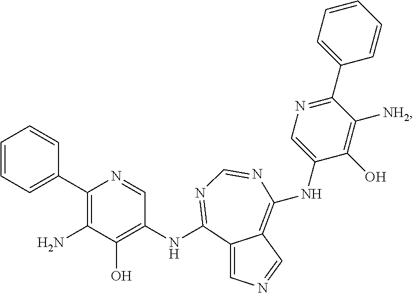 [SMILES: Nc1c(-c2ccccc2)ncc(Nc2ncnc(Nc3cnc(-c4ccccc4)c(N)c3O)c3cncc2-3)c1O] | V-846 |
| 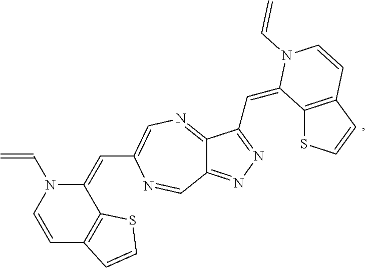 [SMILES: C=CN1C=Cc2ccsc2/C1=C\c1cnc2c(/C=C3\c4sccc4C=CN3C=C)nnc-2cn1] | V-847 |
| 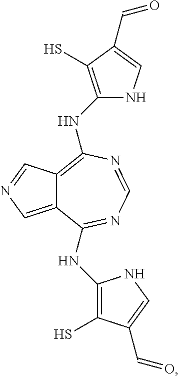 [SMILES: O=Cc1c[nH]c(Nc2ncnc(Nc3[nH]cc(C=O)c3S)c3cncc2-3)c1S] | V-848 |
| 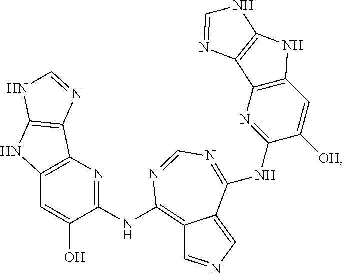 [SMILES: Oc1cc2[nH]c3[nH]cnc3c2nc1Nc1ncnc(Nc2nc3c(cc2O)[nH]c2[nH]cnc23)c2cncc1-2] | V-849 |
| 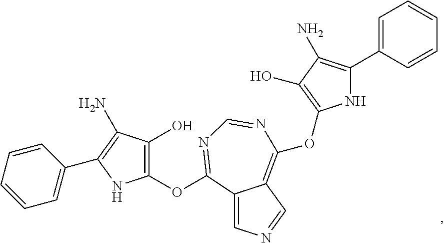 [SMILES: Nc1c(-c2ccccc2)[nH]c(Oc2ncnc(Oc3[nH]c(-c4ccccc4)c(N)c3O)c3cncc2-3)c1O] | V-850 |
| 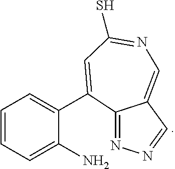 [SMILES: Nc1ccccc1-c1cc(S)ncc2cnnc1-2] | V-851 |
| 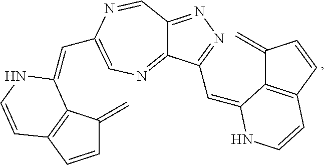 [SMILES: C=c1ccc2cc[nH]/c(=C/c3cnc4c(/C=c5/[nH]ccc6ccc(=C)c5=6)nnc-4cn3)c1=2] | V-852 |
| 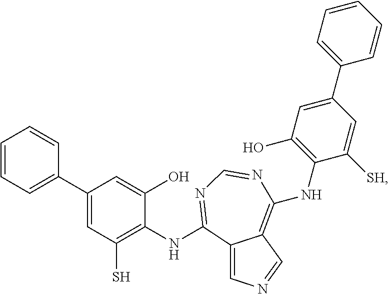 [SMILES: Oc1cc(-c2ccccc2)cc(S)c1Nc1ncnc(Nc2c(O)cc(-c3ccccc3)cc2S)c2cncc1-2] | V-853 |
| 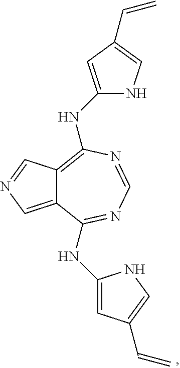 [SMILES: C=Cc1c[nH]c(Nc2ncnc(Nc3cc(C=C)c[nH]3)c3cncc2-3)c1] | V-854 |
| 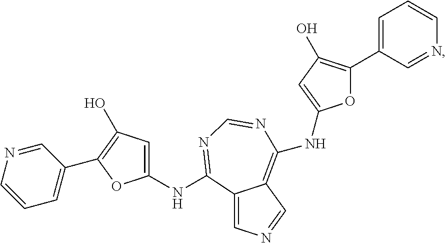 [SMILES: Oc1cc(Nc2ncnc(Nc3cc(O)c(-c4cccnc4)o3)c3cncc2-3)oc1-c1cccnc1] | V-855 |
| 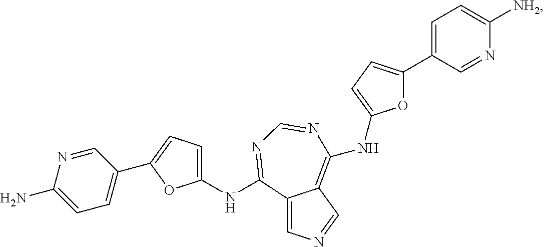 [SMILES: Nc1ccc(-c2ccc(Nc3ncnc(Nc4ccc(-c5ccc(N)nc5)o4)c4cncc3-4)o2)cn1] | V-856 |
| 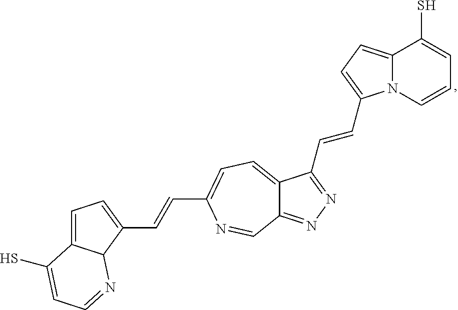 [SMILES: SC1=CC=NC2C(/C=C/c3ccc4c(/C=C/c5ccc6c(S)cccn56)nnc-4cn3)=CC=C12] | V-857 |
| 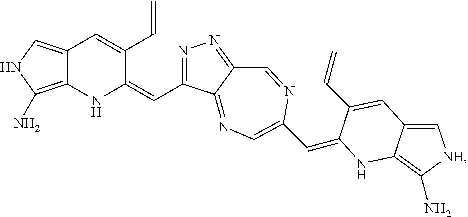 [SMILES: C=CC1=Cc2c[nH]c(N)c2N/C1=C/c1cnc2c(/C=C3/Nc4c(c[nH]c4N)C=C3C=C)nnc-2cn1] | V-858 |
| 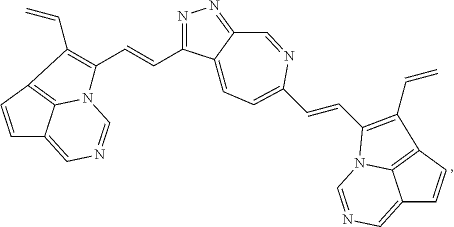 [SMILES: C=Cc1c(/C=C/c2ccc3c(/C=C/c4c(C=C)c5ccc6cncn4c65)nnc-3cn2)n2cncc3ccc1c32] | V-859 |
| 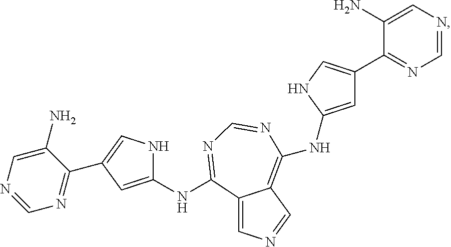 [SMILES: Nc1cncnc1-c1c[nH]c(Nc2ncnc(Nc3cc(-c4ncncc4N)c[nH]3)c3cncc2-3)c1] | V-860 |
| 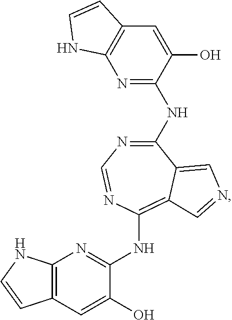 [SMILES: Oc1cc2cc[nH]c2nc1Nc1ncnc(Nc2nc3[nH]ccc3cc2O)c2cncc1-2] | V-861 |
| 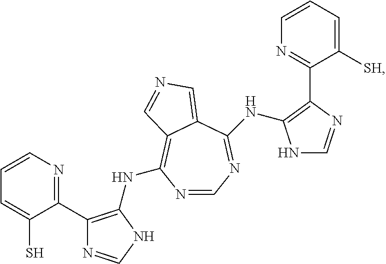 [SMILES: Sc1cccnc1-c1nc[nH]c1Nc1ncnc(Nc2[nH]cnc2-c2ncccc2S)c2cncc1-2] | V-862 |
| 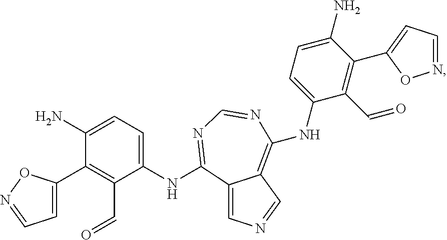 [SMILES: Nc1ccc(Nc2ncnc(Nc3ccc(N)c(-c4ccno4)c3C=O)c3cncc2-3)c(C=O)c1-c1ccno1] | V-863 |
| 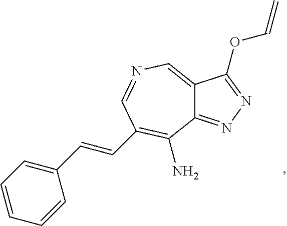 [SMILES: C=COc1nnc2c(N)c(/C=C/c3ccccc3)cncc1-2] | V-864 |
| 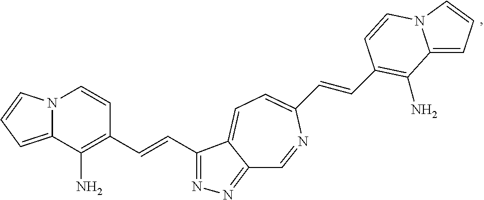 [SMILES: Nc1c(/C=C/c2ccc3c(/C=C/c4ccn5cccc5c4N)nnc-3cn2)ccn2cccc12] | V-865 |
| 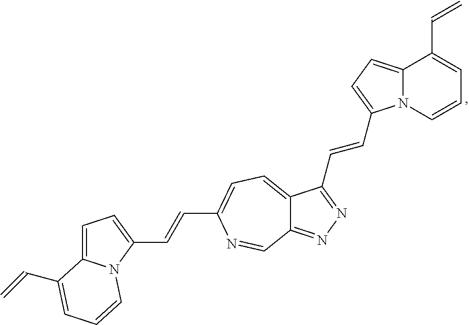 [SMILES: C=Cc1cccn2c(/C=C/c3ccc4c(/C=C/c5ccc6c(C=C)cccn56)nnc-4cn3)ccc12] | V-866 |
| 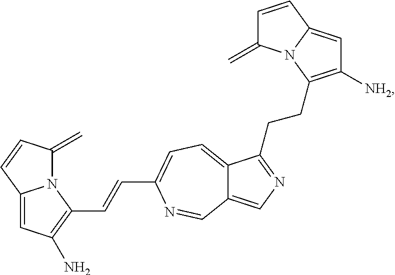 [SMILES: C=c1ccc2cc(N)c(/C=C/c3ccc4c(CCc5c(N)cc6ccc(=C)n56)ncc-4cn3)n12] | V-867 |
| 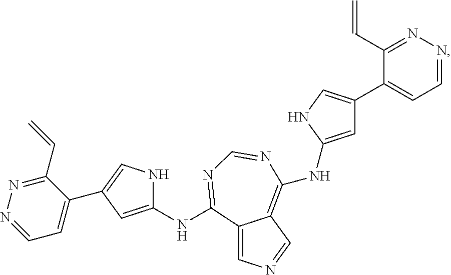 [SMILES: C=Cc1nnccc1-c1c[nH]c(Nc2ncnc(Nc3cc(-c4ccnnc4C=C)c[nH]3)c3cncc2-3)c1] | V-868 |
| 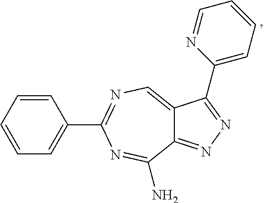 [SMILES: Nc1nc(-c2ccccc2)ncc2c(-c3ccccn3)nnc1-2] | V-869 |
| 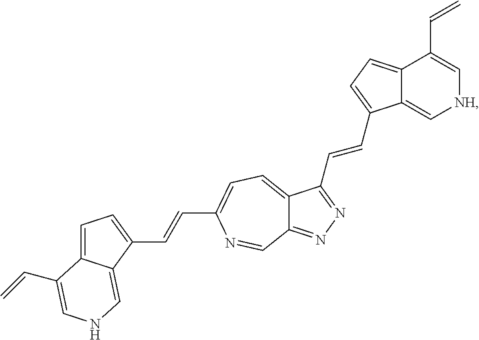 [SMILES: C=Cc1c[nH]cc2c(/C=C/c3ccc4c(/C=C/c5ccc6c(C=C)c[nH]cc5-6)nnc-4cn3)ccc1-2] | V-870 |
| 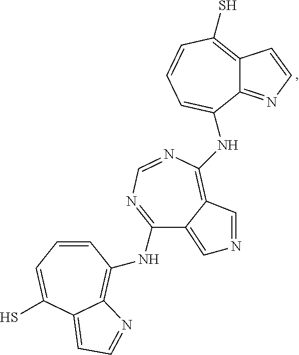 [SMILES: Sc1cccc(Nc2ncnc(Nc3cccc(S)c4ccnc3-4)c3cncc2-3)c2nccc1-2] | V-871 |
| 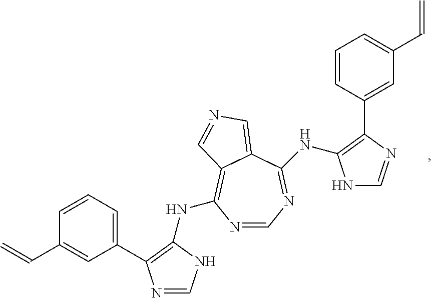 [SMILES: C=Cc1cccc(-c2nc[nH]c2Nc2ncnc(Nc3[nH]cnc3-c3cccc(C=C)c3)c3cncc2-3)c1] | V-872 |
| 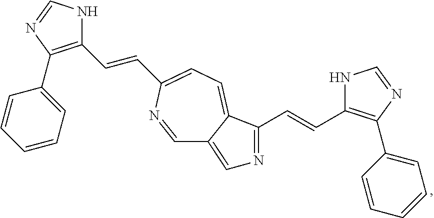 [SMILES: C(=C\c1[nH]cnc1-c1ccccc1)/c1ccc2c(/C=C/c3[nH]cnc3-c3ccccc3)ncc-2cn1] | V-873 |
| 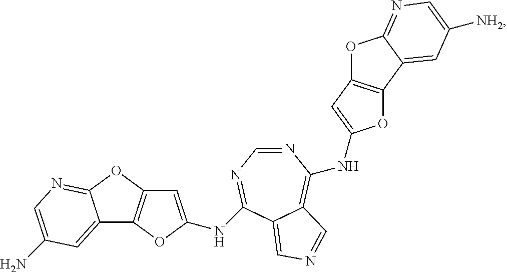 [SMILES: Nc1cnc2oc3cc(Nc4ncnc(Nc5cc6oc7ncc(N)cc7c6o5)c5cncc4-5)oc3c2c1] | V-874 |
| 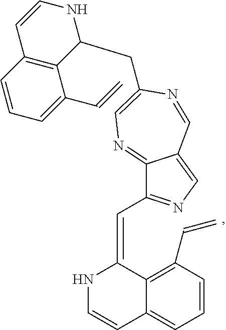 [SMILES: C=Cc1cccc2c1/C(=C\c1ncc3cnc(CC4NC=Cc5cccc(C=C)c54)cnc1-3)NC=C2] | V-875 |
| 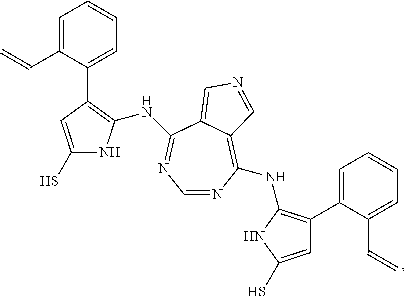 [SMILES: C=Cc1ccccc1-c1cc(S)[nH]c1Nc1ncnc(Nc2[nH]c(S)cc2-c2ccccc2C=C)c2cncc1-2] | V-876 |
| 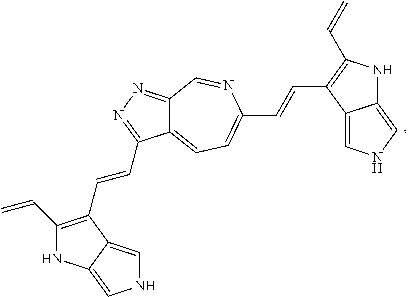 [SMILES: C=Cc1[nH]c2c[nH]cc2c1/C=C/c1ccc2c(/C=C/c3c(C=C)[nH]c4c[nH]cc34)nnc-2cn1] | V-877 |
| 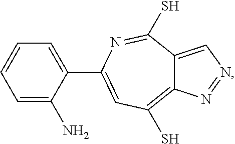 [SMILES: Nc1ccccc1-c1cc(S)c2nncc-2c(S)n1] | V-878 |
| 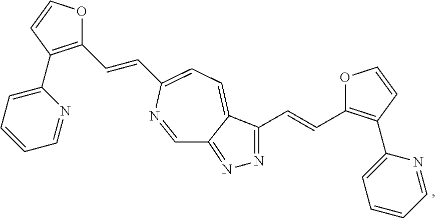 [SMILES: C(=C\c1occc1-c1ccccn1)/c1ccc2c(/C=C/c3occc3-c3ccccn3)nnc-2cn1] | V-879 |
| 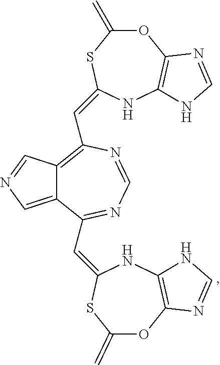 [SMILES: C=c1oc2nc[nH]c2[nH]/c(=C\c2ncnc(/C=c3\[nH]c4[nH]cnc4oc(=C)s3)c3cncc2-3)s1] | V-880 |
| 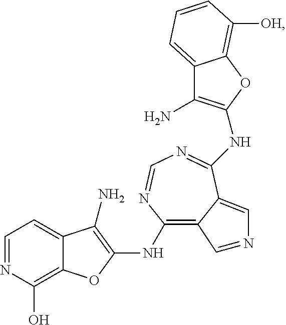 [SMILES: Nc1c(Nc2ncnc(Nc3oc4c(O)nccc4c3N)c3cncc2-3)oc2c(O)cccc12] | V-881 |
| 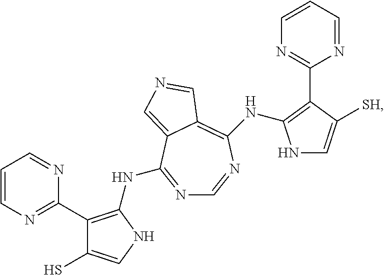 [SMILES: Sc1c[nH]c(Nc2ncnc(Nc3[nH]cc(S)c3-c3ncccn3)c3cncc2-3)c1-c1ncccn1] | V-882 |
| 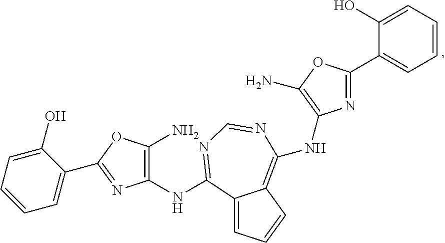 [SMILES: Nc1oc(-c2ccccc2O)nc1Nc1ncnc(Nc2nc(-c3ccccc3O)oc2N)c2cccc1-2] | V-883 |
| 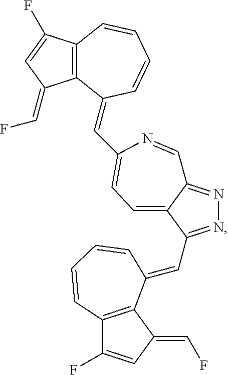 [SMILES: F/C=c1\cc(F)c2cccc/c(=C\c3ccc4c(/C=c5\ccccc6c(F)c/c(=C\F)c5=6)nnc-4cn3)c1=2] | V-884 |
| 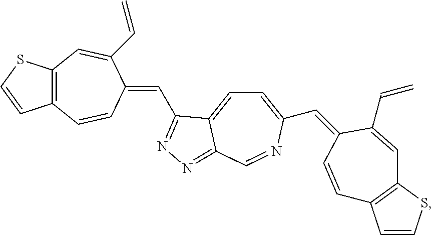 [SMILES: C=CC1=Cc2sccc2C=C/C1=C\c1ccc2c(/C=C3\C=Cc4ccsc4C=C3C=C)nnc-2cn1] | V-885 |
| 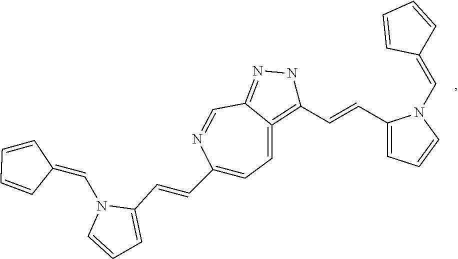 | V-886 |
| 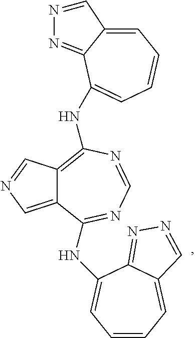 [SMILES: c1ccc(Nc2ncnc(Nc3ccccc4cnnc3-4)c3cncc2-3)c2nncc-2c1] | V-887 |
| 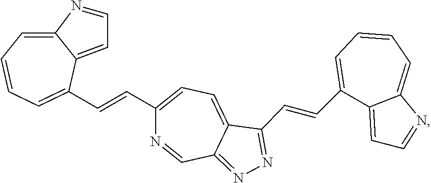 [SMILES: C(=C\c1ccccc2nccc1-2)/c1ccc2c(/C=C/c3ccccc4nccc3-4)nnc-2cn1] | V-888 |
| 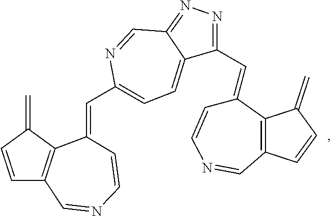 [SMILES: C=c1ccc2cncc/c(=C\c3ccc4c(/C=c5\ccncc6ccc(=C)c5=6)nnc-4cn3)c1=2] | V-889 |
| 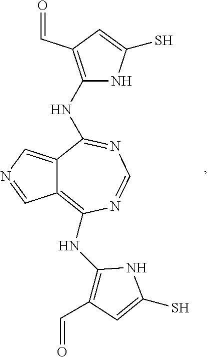 [SMILES: O=Cc1cc(S)[nH]c1Nc1ncnc(Nc2[nH]c(S)cc2C=O)c2cncc1-2] | V-890 |
| 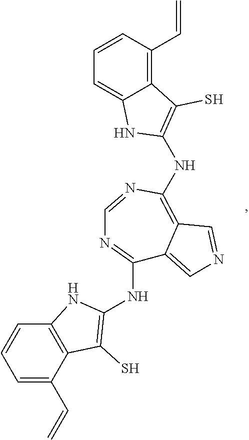 [SMILES: C=Cc1cccc2[nH]c(Nc3ncnc(Nc4[nH]c5cccc(C=C)c5c4S)c4cncc3-4)c(S)c12] | V-891 |
| 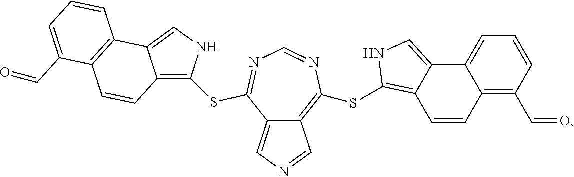 [SMILES: O=Cc1cccc2c1ccc1c(Sc3ncnc(Sc4[nH]cc5c4ccc4c(C=O)cccc45)c4cncc3-4)[nH]cc12] | V-892 |
| 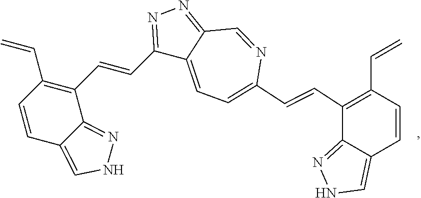 [SMILES: C=Cc1ccc2c[nH]nc2c1/C=C/c1ccc2c(/C=C/c3c(C=C)ccc4c[nH]nc34)nnc-2cn1] | V-893 |
| 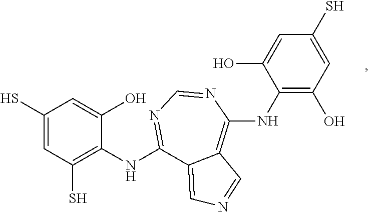 [SMILES: Oc1cc(S)cc(O)c1Nc1ncnc(Nc2c(O)cc(S)cc2S)c2cncc1-2] | V-894 |
| 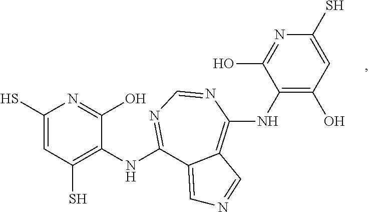 [SMILES: Oc1cc(S)nc(O)c1Nc1ncnc(Nc2c(S)cc(S)nc2O)c2cncc1-2] | V-895 |
| 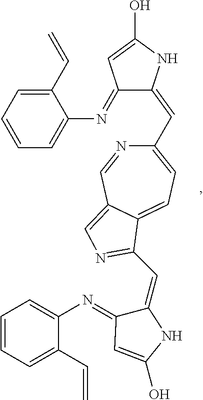 [SMILES: C=Cc1ccccc1/N=C1\C=C(O)N\C1=C\c1ccc2c(/C=C3/NC(O)=C/C3=N\c3ccccc3C=C)ncc-2cn1] | V-896 |
| 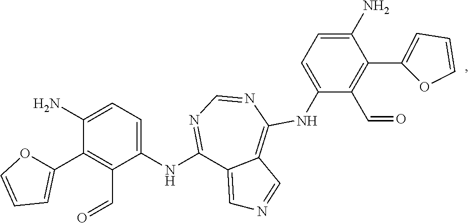 [SMILES: Nc1ccc(Nc2ncnc(Nc3ccc(N)c(-c4ccco4)c3C=O)c3cncc2-3)c(C=O)c1-c1ccco1] | V-897 |
| 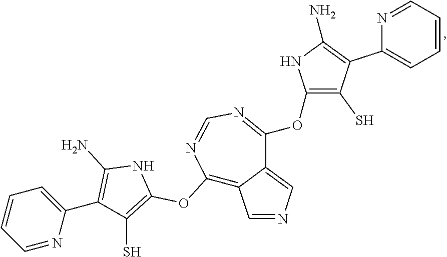 [SMILES: Nc1[nH]c(Oc2ncnc(Oc3[nH]c(N)c(-c4ccccn4)c3S)c3cncc2-3)c(S)c1-c1ccccn1] | V-898 |
| 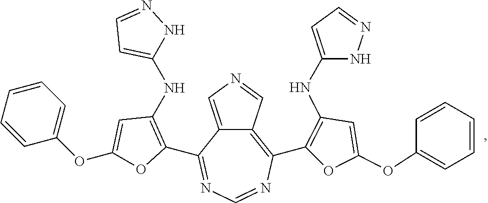 [SMILES: c1ccc(Oc2cc(Nc3ccn[nH]3)c(-c3ncnc(-c4oc(Oc5ccccc5)cc4Nc4ccn[nH]4)c4cncc3-4)o2)cc1] | V-899 |
| 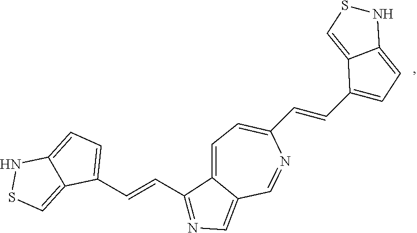 [SMILES: C(=C\c1ccc2[nH]scc1-2)/c1ccc2c(/C=C/c3ccc4[nH]scc3-4)ncc-2cn1] | V-900 |
| 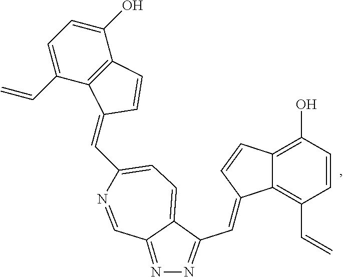 [SMILES: C=Cc1ccc(O)c2c1/C(=C/c1ccc3c(/C=C4\C=Cc5c(O)ccc(C=C)c54)nnc-3cn1)C=C2] | V-901 |
| 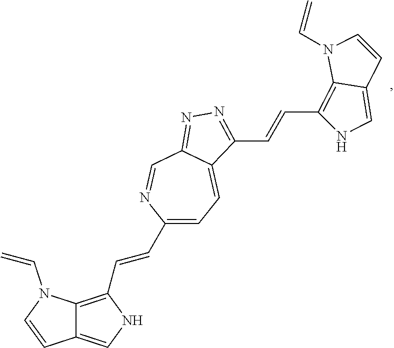 [SMILES: C=Cn1ccc2c[nH]c(/C=C/c3ccc4c(/C=C/c5[nH]cc6ccn(C=C)c56)nnc-4cn3)c21] | V-902 |
| 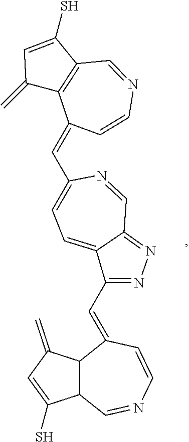 [SMILES: C=C1C=C(S)C2C=NC=C/C(=C\c3nnc4cnc(/C=c5\ccncc6c(S)cc(=C)c5=6)ccc3-4)C12] | V-903 |
| 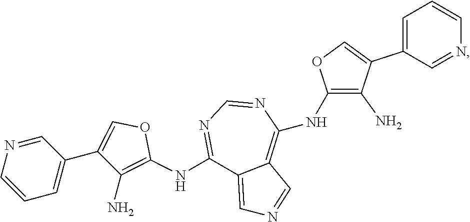 [SMILES: Nc1c(-c2cccnc2)coc1Nc1ncnc(Nc2occ(-c3cccnc3)c2N)c2cncc1-2] | V-904 |
| 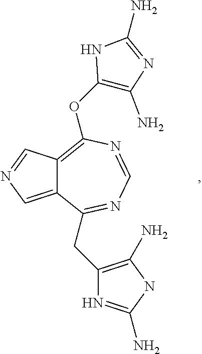 [SMILES: Nc1nc(Cc2ncnc(Oc3[nH]c(N)nc3N)c3cncc2-3)c(N)[nH]1] | V-905 |
| 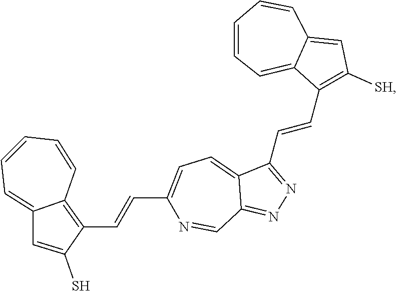 [SMILES: Sc1cc2cccccc-2c1/C=C/c1ccc2c(/C=C/c3c(S)cc4cccccc3-4)nnc-2cn1] | V-906 |
| 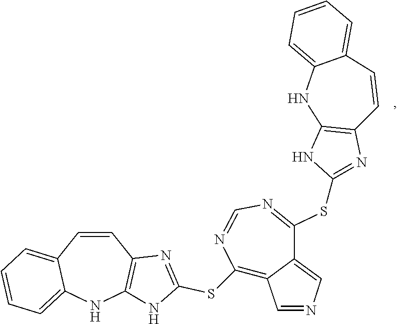 [SMILES: C1=Cc2nc(Sc3ncnc(Sc4nc5c([nH]4)Nc4ccccc4C=C5)c4cncc3-4)[nH]c2Nc2ccccc21] | V-907 |
| 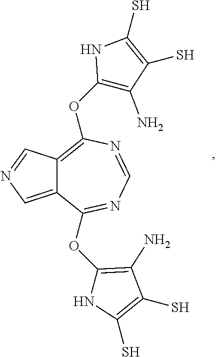 [SMILES: Nc1c(Oc2ncnc(Oc3[nH]c(S)c(S)c3N)c3cncc2-3)[nH]c(S)c1S] | V-908 |
| 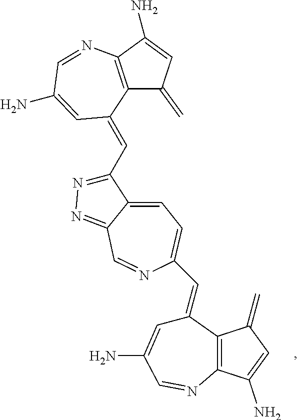 [SMILES: C=c1cc(N)c2ncc(N)c/c(=C\c3ccc4c(/C=c5\cc(N)cnc6c(N)cc(=C)c5=6)nnc-4cn3)c1=2] | V-909 |
| 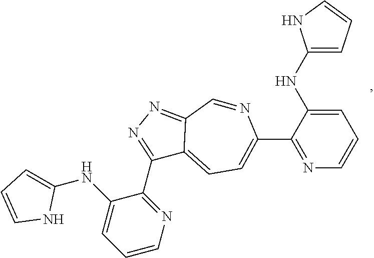 [SMILES: c1c[nH]c(Nc2cccnc2-c2ccc3c(-c4ncccc4Nc4ccc[nH]4)nnc-3cn2)c1] | V-910 |
| 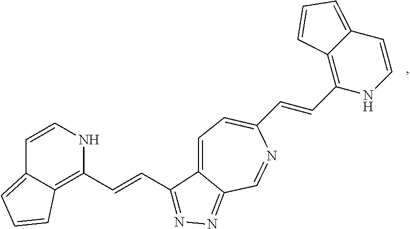 [SMILES: C(=C\c1[nH]ccc2cccc1-2)/c1ccc2c(/C=C/c3[nH]ccc4cccc3-4)nnc-2cn1] | V-911 |
| 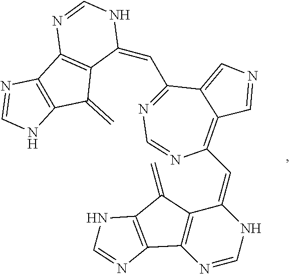 [SMILES: C=c1c2/c(=C\c3ncnc(/C=c4/[nH]cnc5c6nc[nH]c6c(=C)c4=5)c4cncc3-4)[nH]cnc=2c2nc[nH]c12] | V-912 |
| 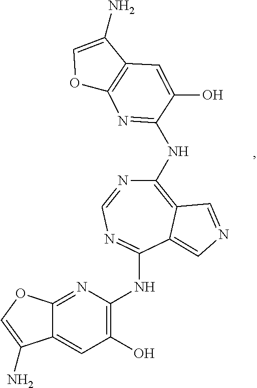 [SMILES: Nc1coc2nc(Nc3ncnc(Nc4nc5occ(N)c5cc4O)c4cncc3-4)c(O)cc12] | V-913 |
| 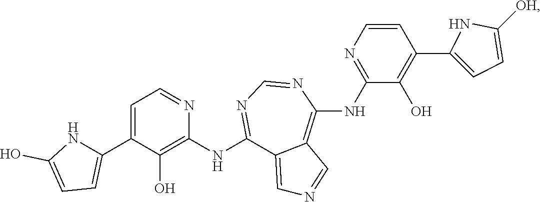 [SMILES: Oc1ccc(-c2ccnc(Nc3ncnc(Nc4nccc(-c5ccc(O)[nH]5)c4O)c4cncc3-4)c2O)[nH]1] | V-914 |
| 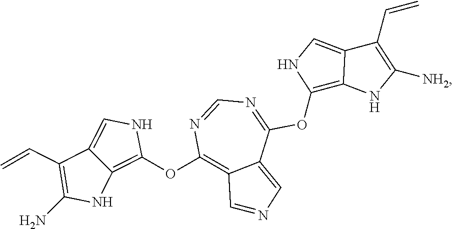 [SMILES: C=Cc1c(N)[nH]c2c(Oc3ncnc(Oc4[nH]cc5c(C=C)c(N)[nH]c45)c4cncc3-4)[nH]cc12] | V-915 |
| 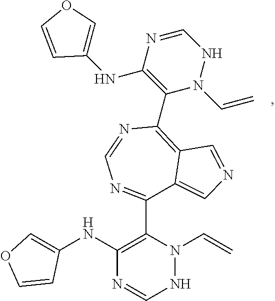 [SMILES: C=CN1NC=NC(Nc2ccoc2)=C1c1ncnc(C2=C(Nc3ccoc3)N=CNN2C=C)c2cncc1-2] | V-916 |
| 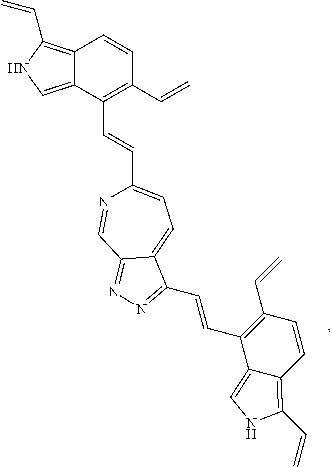 [SMILES: C=Cc1ccc2c(C=C)[nH]cc2c1/C=C/c1ccc2c(/C=C/c3c(C=C)ccc4c(C=C)[nH]cc34)nnc-2cn1] | V-917 |
| 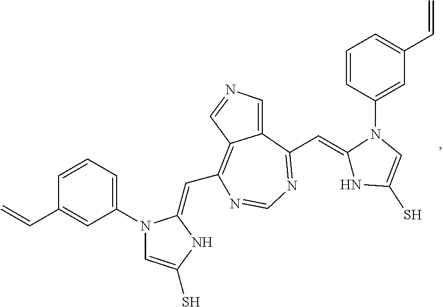 [SMILES: C=Cc1cccc(N2C=C(S)N/C2=C\c2ncnc(/C=C3\NC(S)=CN3c3cccc(C=C)c3)c3cncc2-3)c1] | V-918 |
| 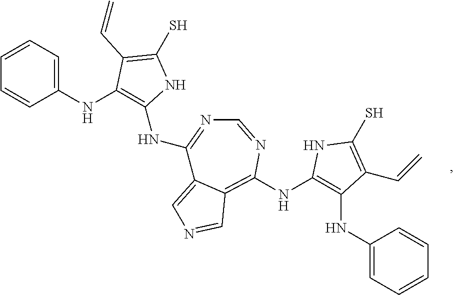 [SMILES: C=Cc1c(S)[nH]c(Nc2ncnc(Nc3[nH]c(S)c(C=C)c3Nc3ccccc3)c3cncc2-3)c1Nc1ccccc1] | V-919 |
| 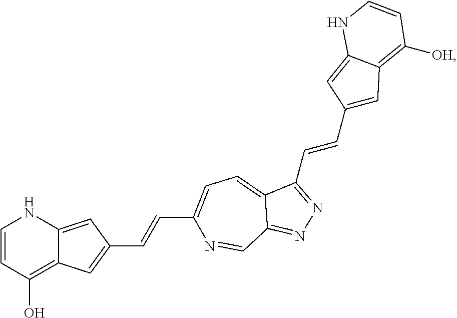 [SMILES: Oc1cc[nH]c2cc(/C=C/c3ccc4c(/C=C/c5cc6[nH]ccc(O)c-6c5)nnc-4cn3)cc1-2] | V-920 |
| 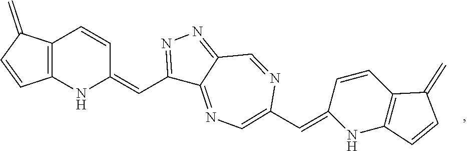 [SMILES: C=c1ccc2[nH]/c(=C/c3cnc4c(/C=c5\ccc6c(=C)ccc=6[nH]5)nnc-4cn3)ccc1=2] | V-921 |
| 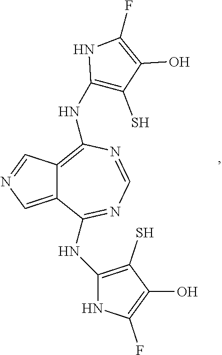 [SMILES: Oc1c(F)[nH]c(Nc2ncnc(Nc3[nH]c(F)c(O)c3S)c3cncc2-3)c1S] | V-922 |
| 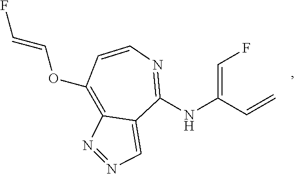 [SMILES: C=C/C(=C\F)Nc1nccc(O/C=C/F)c2nncc1-2] | V-923 |
| 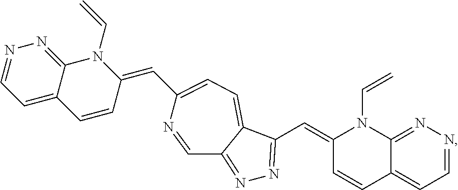 [SMILES: C=CN1/C(=C/c2ccc3c(/C=C4\C=Cc5ccnnc5N4C=C)nnc-3cn2)C=Cc2ccnnc21] | V-924 |
| 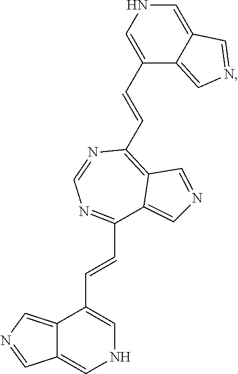 [SMILES: C(=C\c1ncnc(/C=C/c2c[nH]cc3cncc2-3)c2cncc1-2)/c1c[nH]cc2cncc1-2] | V-925 |
| 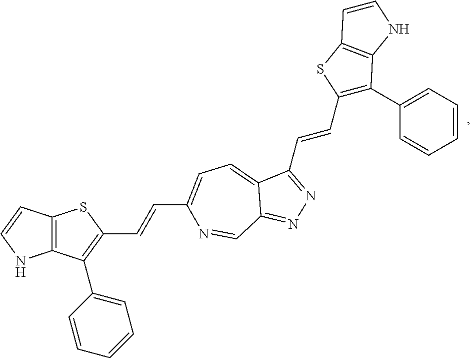 [SMILES: C(=C\c1sc2cc[nH]c2c1-c1ccccc1)/c1ccc2c(/C=C/c3sc4cc[nH]c4c3-c3ccccc3)nnc-2cn1] | V-926 |
| 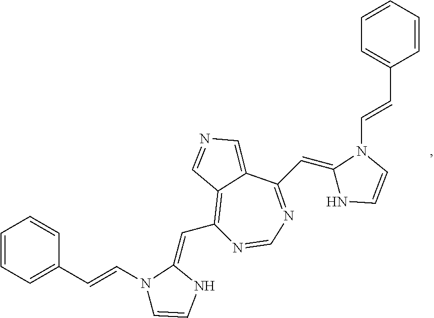 [SMILES: C1=CN(/C=C/c2ccccc2)/C(=C/c2ncnc(/C=C3\NC=CN3/C=C/c3ccccc3)c3cncc2-3)N1] | V-927 |
| 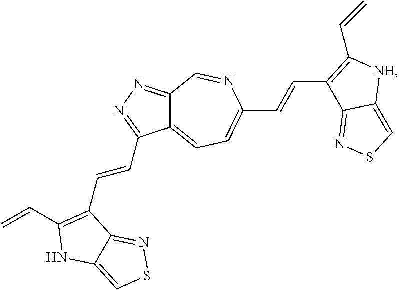 [SMILES: C=Cc1[nH]c2csnc2c1/C=C/c1ccc2c(/C=C/c3c(C=C)[nH]c4csnc34)nnc-2cn1] | V-928 |
| 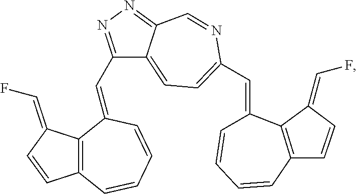 [SMILES: F/C=c1\ccc2cccc/c(=C\c3ccc4c(/C=c5\ccccc6cc/c(=C\F)c5=6)nnc-4cn3)c1=2] | V-929 |
| 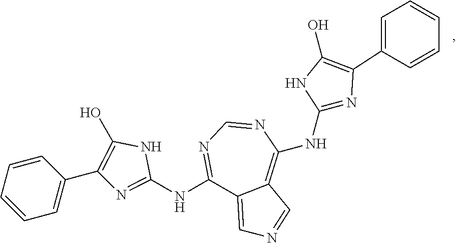 [SMILES: Oc1[nH]c(Nc2ncnc(Nc3nc(-c4ccccc4)c(O)[nH]3)c3cncc2-3)nc1-c1ccccc1] | V-930 |
| 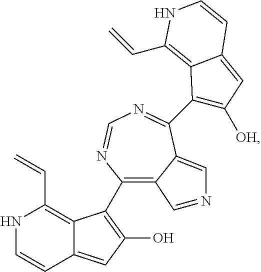 [SMILES: C=Cc1[nH]ccc2cc(O)c(-c3ncnc(-c4c(O)cc5cc[nH]c(C=C)c4-5)c4cncc3-4)c1-2] | V-931 |
| 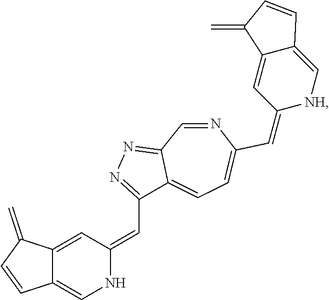 [SMILES: C=c1ccc2c[nH]/c(=C/c3ccc4c(/C=c5\cc6c(=C)ccc-6c[nH]5)nnc-4cn3)cc1-2] | V-932 |
| 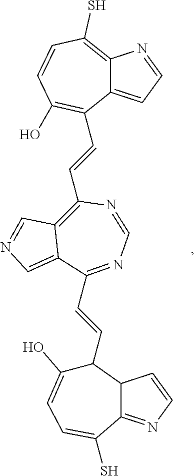 [SMILES: OC1=CC=C(S)C2=NC=CC2C1/C=C/c1ncnc(/C=C/c2c(O)ccc(S)c3nccc2-3)c2cncc1-2] | V-933 |
| 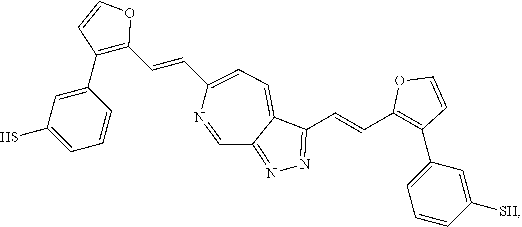 [SMILES: Sc1cccc(-c2ccoc2/C=C/c2ccc3c(/C=C/c4occc4-c4cccc(S)c4)nnc-3cn2)c1] | V-934 |
| 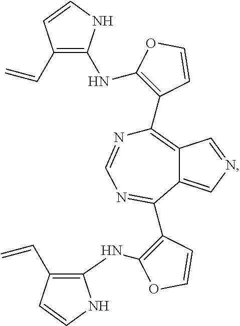 [SMILES: C=Cc1cc[nH]c1Nc1occc1-c1ncnc(-c2ccoc2Nc2[nH]ccc2C=C)c2cncc1-2] | V-935 |
| 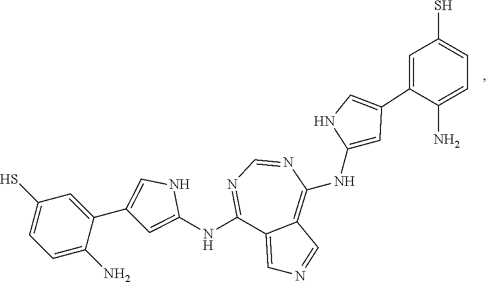 [SMILES: Nc1ccc(S)cc1-c1c[nH]c(Nc2ncnc(Nc3cc(-c4cc(S)ccc4N)c[nH]3)c3cncc2-3)c1] | V-936 |
| 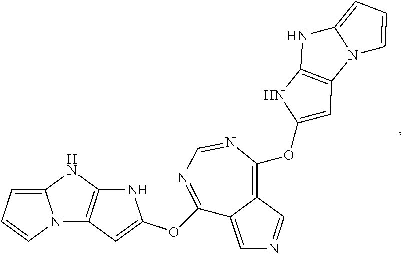 [SMILES: c1cc2[nH]c3[nH]c(Oc4ncnc(Oc5cc6c([nH]5)[nH]c5cccn56)c5cncc4-5)cc3n2c1] | V-937 |
| 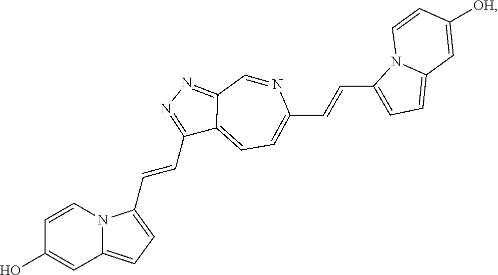 [SMILES: Oc1ccn2c(/C=C/c3ccc4c(/C=C/c5ccc6cc(O)ccn56)nnc-4cn3)ccc2c1] | V-938 |
| 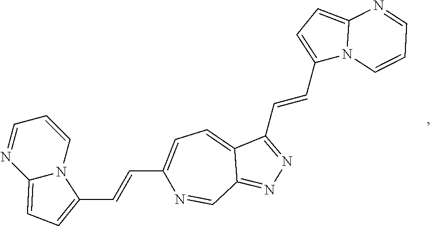 [SMILES: C(=C\c1ccc2ncccn12)/c1ccc2c(/C=C/c3ccc4ncccn34)nnc-2cn1] | V-939 |
| 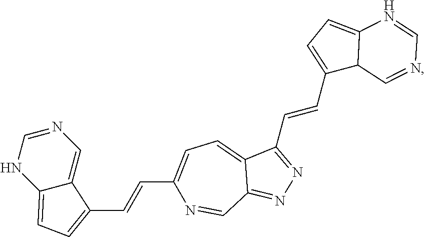 | V-940 |
| 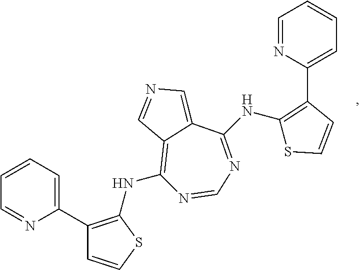 [SMILES: c1ccc(-c2ccsc2Nc2ncnc(Nc3sccc3-c3ccccn3)c3cncc2-3)nc1] | V-941 |
| 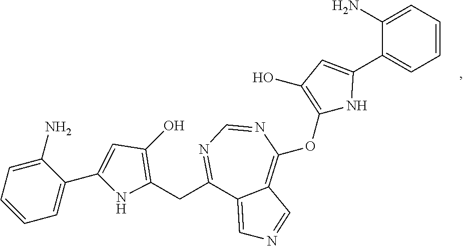 [SMILES: Nc1ccccc1-c1cc(O)c(Cc2ncnc(Oc3[nH]c(-c4ccccc4N)cc3O)c3cncc2-3)[nH]1] | V-942 |
| 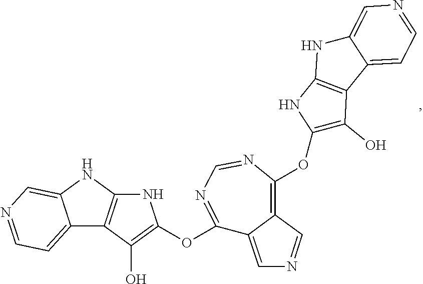 [SMILES: Oc1c(Oc2ncnc(Oc3[nH]c4[nH]c5cnccc5c4c3O)c3cncc2-3)[nH]c2[nH]c3cnccc3c12] | V-943 |
| 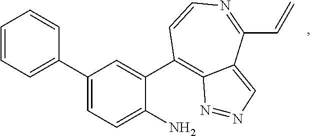 [SMILES: C=Cc1nccc(-c2cc(-c3ccccc3)ccc2N)c2nncc1-2] | V-944 |
| 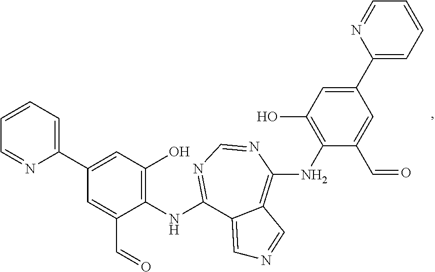 [SMILES: O=Cc1cc(-c2ccccn2)cc(O)c1Nc1ncnc(Nc2c(O)cc(-c3ccccn3)cc2C=O)c2cncc1-2] | V-945 |
| 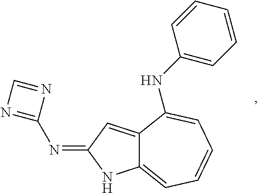 [SMILES: C1=NC(/N=c2\cc3c(Nc4ccccc4)ccccc-3[nH]2)=N1] | V-946 |
| 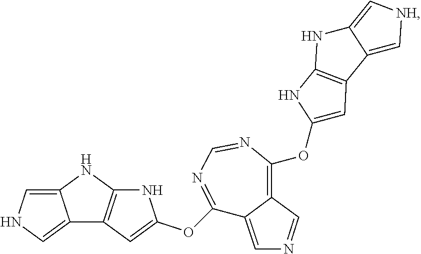 [SMILES: c1nc(Oc2cc3c([nH]2)[nH]c2c[nH]cc23)c2cncc-2c(Oc2cc3c([nH]2)[nH]c2c[nH]cc23)n1] | V-947 |
| 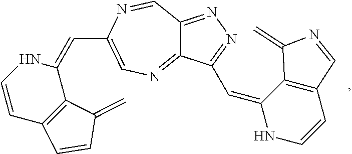 [SMILES: C=c1ccc2cc[nH]/c(=C/c3cnc4c(/C=c5/[nH]ccc6cnc(=C)c5=6)nnc-4cn3)c1=2] | V-948 |
| 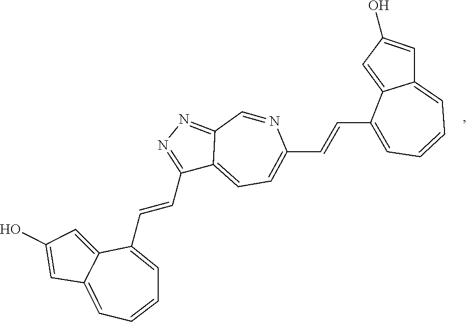 [SMILES: Oc1cc2ccccc(/C=C/c3ccc4c(/C=C/c5ccccc6cc(O)cc5-6)nnc-4cn3)c-2c1] | V-949 |
| 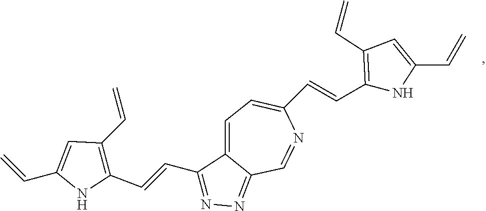 [SMILES: C=Cc1cc(C=C)c(/C=C/c2ccc3c(/C=C/c4[nH]c(C=C)cc4C=C)nnc-3cn2)[nH]1] | V-950 |
| 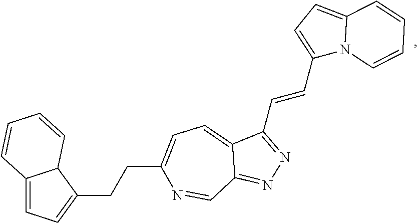 [SMILES: C1=CC2=CC=C(CCc3ccc4c(/C=C/c5ccc6ccccn56)nnc-4cn3)C2C=C1] | V-951 |
| 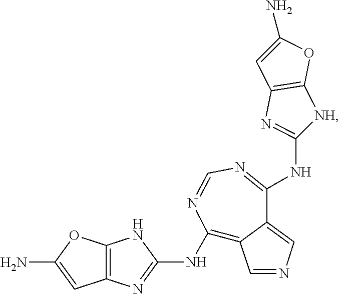 [SMILES: Nc1cc2nc(Nc3ncnc(Nc4nc5cc(N)oc5[nH]4)c4cncc3-4)[nH]c2o1] | V-952 |
| 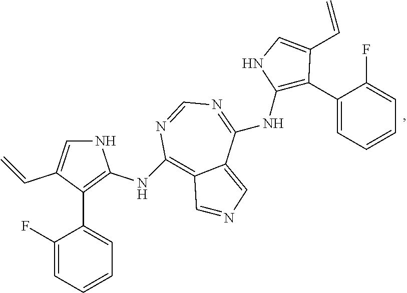 [SMILES: C=Cc1c[nH]c(Nc2ncnc(Nc3[nH]cc(C=C)c3-c3ccccc3F)c3cncc2-3)c1-c1ccccc1F] | V-953 |
| 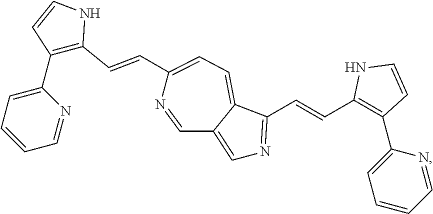 [SMILES: C(=C\c1[nH]ccc1-c1ccccn1)/c1ccc2c(/C=C/c3[nH]ccc3-c3ccccn3)ncc-2cn1] | V-954 |
| 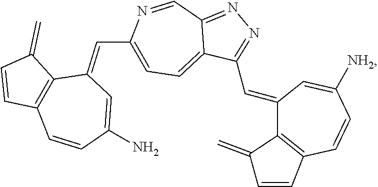 [SMILES: C=c1ccc2ccc(N)c/c(=C\c3ccc4c(/C=c5\cc(N)ccc6ccc(=C)c5=6)nnc-4cn3)c1=2] | V-955 |
| 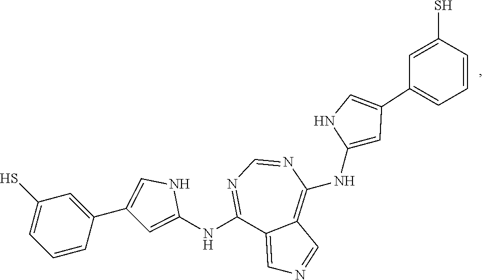 [SMILES: Sc1cccc(-c2c[nH]c(Nc3ncnc(Nc4cc(-c5cccc(S)c5)c[nH]4)c4cncc3-4)c2)c1] | V-956 |
| 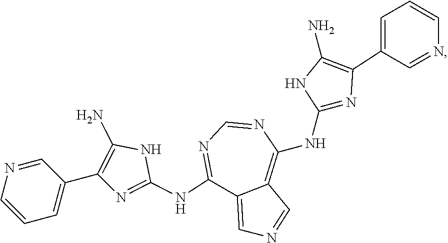 [SMILES: Nc1[nH]c(Nc2ncnc(Nc3nc(-c4cccnc4)c(N)[nH]3)c3cncc2-3)nc1-c1cccnc1] | V-957 |
| 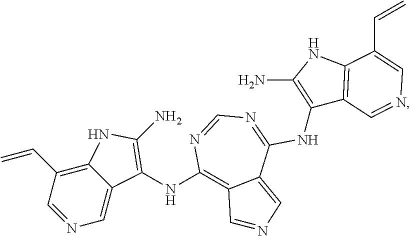 [SMILES: C=Cc1cncc2c(Nc3ncnc(Nc4c(N)[nH]c5c(C=C)cncc45)c4cncc3-4)c(N)[nH]c12] | V-958 |
| 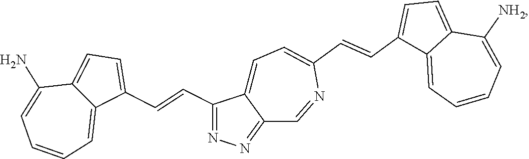 [SMILES: Nc1ccccc2c(/C=C/c3ccc4c(/C=C/c5ccc6c(N)ccccc5-6)nnc-4cn3)ccc1-2] | V-959 |
| 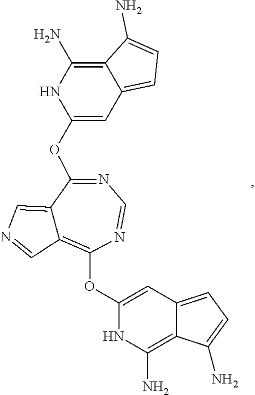 [SMILES: Nc1ccc2cc(Oc3ncnc(Oc4cc5ccc(N)c-5c(N)[nH]4)c4cncc3-4)[nH]c(N)c1-2] | V-960 |
| 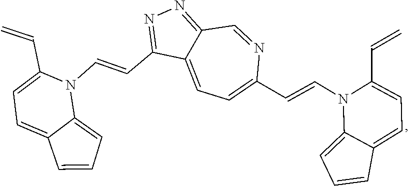 [SMILES: C=Cc1ccc2cccc-2n1/C=C/c1ccc2c(/C=C/n3c(C=C)ccc4cccc3-4)nnc-2cn1] | V-961 |
| 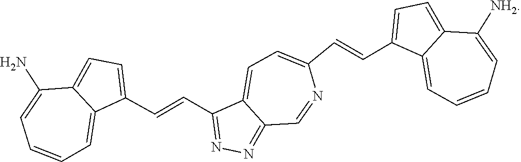 [SMILES: Nc1ccccc2c(/C=C/c3ccc4c(/C=C/c5ccc6c(N)ccccc5-6)nnc-4cn3)ccc1-2] | V-962 |
| 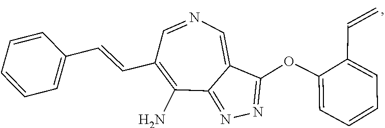 [SMILES: C=Cc1ccccc1Oc1nnc2c(N)c(/C=C/c3ccccc3)cncc1-2] | V-963 |
| 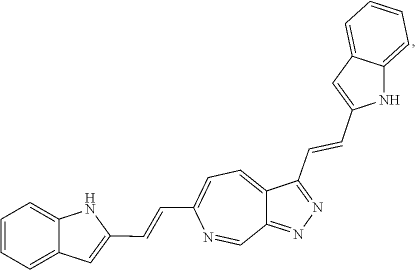 [SMILES: C(=C\c1cc2ccccc2[nH]1)/c1ccc2c(/C=C/c3cc4ccccc4[nH]3)nnc-2cn1] | V-964 |
| 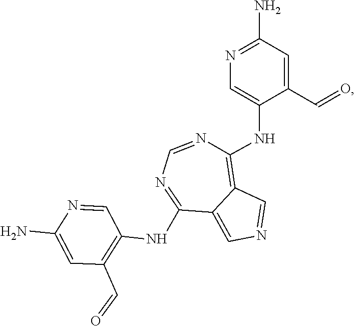 [SMILES: Nc1cc(C=O)c(Nc2ncnc(Nc3cnc(N)cc3C=O)c3cncc2-3)cn1] | V-965 |
| 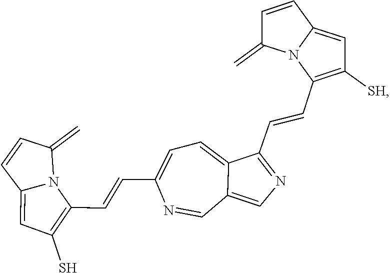 [SMILES: C=c1ccc2cc(S)c(/C=C/c3ccc4c(/C=C/c5c(S)cc6ccc(=C)n56)ncc-4cn3)n12] | V-966 |
| 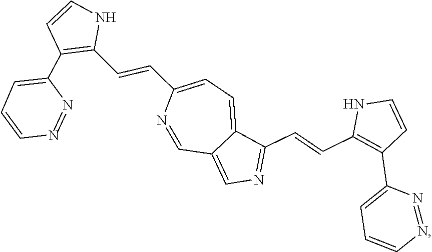 | V-967 |
| 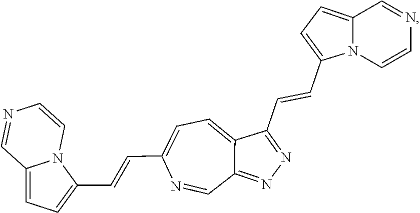 [SMILES: C(=C\c1ccc2cnccn12)/c1ccc2c(/C=C/c3ccc4cnccn34)nnc-2cn1] | V-968 |
| 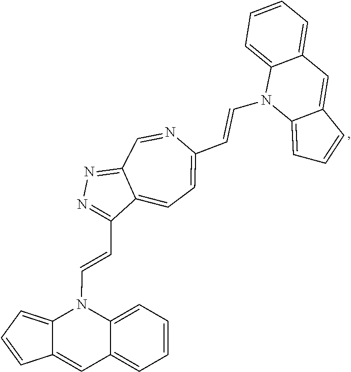 [SMILES: C(=C\n1c2cccc-2cc2ccccc21)/c1ccc2c(/C=C/n3c4cccc-4cc4ccccc43)nnc-2cn1] | V-969 |
| 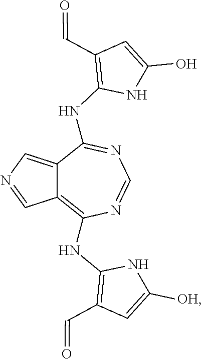 [SMILES: O=Cc1cc(O)[nH]c1Nc1ncnc(Nc2[nH]c(O)cc2C=O)c2cncc1-2] | V-970 |
| 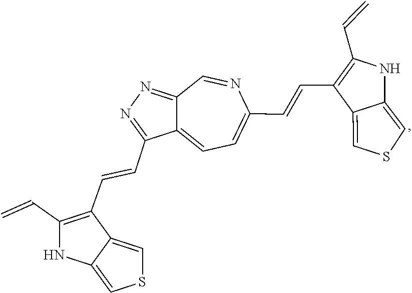 [SMILES: C=Cc1[nH]c2cscc2c1/C=C/c1ccc2c(/C=C/c3c(C=C)[nH]c4cscc34)nnc-2cn1] | V-971 |
| 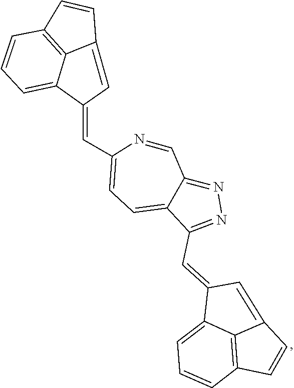 [SMILES: C(/c1ccc2c(/C=c3\cc4ccc5cccc3c5-4)nnc-2cn1)=c1/cc2ccc3cccc1c3-2] | V-972 |
| 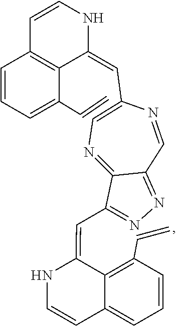 [SMILES: C=Cc1cccc2c1/C(=C\c1cnc3c(/C=C4/NC=Cc5cccc(C=C)c54)nnc-3cn1)NC=C2] | V-973 |
| 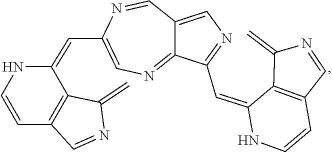 [SMILES: C=c1ncc2cc[nH]/c(=C/c3cnc4c(/C=c5/[nH]ccc6cnc(=C)c5=6)ncc-4cn3)c1=2] | V-974 |
| 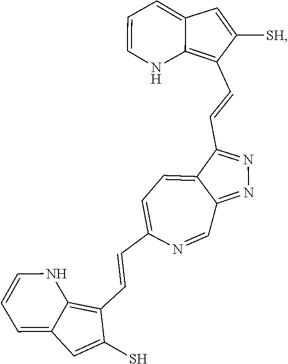 [SMILES: Sc1cc2ccc[nH]c-2c1/C=C/c1ccc2c(/C=C/c3c(S)cc4ccc[nH]c3-4)nnc-2cn1] | V-975 |
| 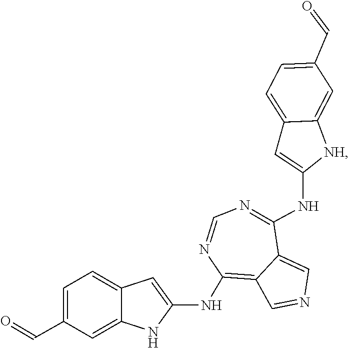 [SMILES: O=Cc1ccc2cc(Nc3ncnc(Nc4cc5ccc(C=O)cc5[nH]4)c4cncc3-4)[nH]c2c1] | V-976 |
| 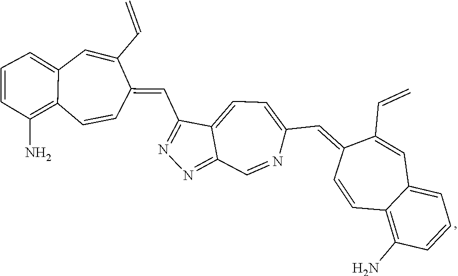 | V-977 |
| 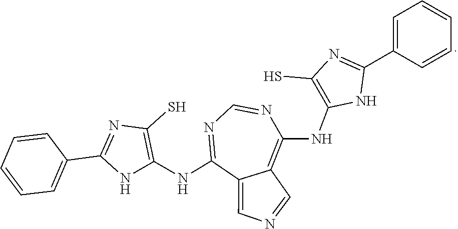 [SMILES: Sc1nc(-c2ccccc2)[nH]c1Nc1ncnc(Nc2[nH]c(-c3ccccc3)nc2S)c2cncc1-2] | V-978 |
| 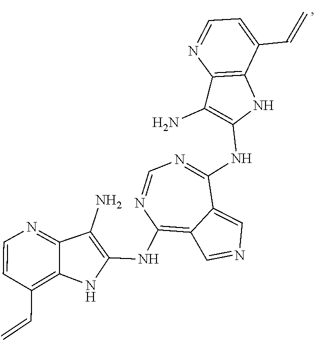 [SMILES: C=Cc1ccnc2c(N)c(Nc3ncnc(Nc4[nH]c5c(C=C)ccnc5c4N)c4cncc3-4)[nH]c12] | V-979 |
| 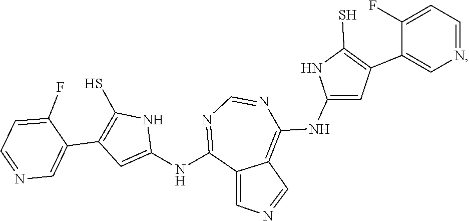 [SMILES: Fc1ccncc1-c1cc(Nc2ncnc(Nc3cc(-c4cnccc4F)c(S)[nH]3)c3cncc2-3)[nH]c1S] | V-980 |
| 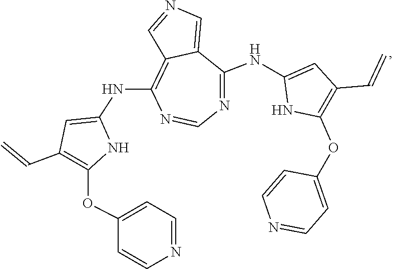 [SMILES: C=Cc1cc(Nc2ncnc(Nc3cc(C=C)c(Oc4ccncc4)[nH]3)c3cncc2-3)[nH]c1Oc1ccncc1] | V-981 |
| 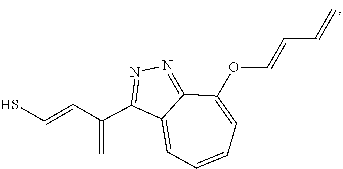 [SMILES: C=C/C=C/Oc1ccccc2c(C(=C)/C=C/S)nnc1-2] | V-982 |
| 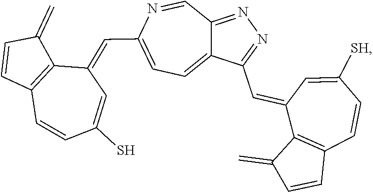 [SMILES: C=c1ccc2ccc(S)c/c(=C\c3ccc4c(/C=c5\cc(S)ccc6ccc(=C)c5=6)nnc-4cn3)c1=2] | V-983 |
| 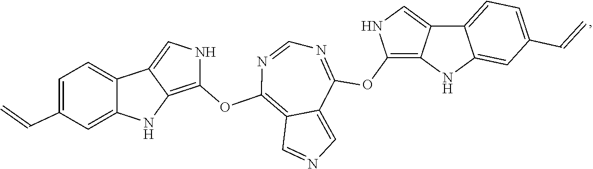 [SMILES: C=Cc1ccc2c(c1)[nH]c1c(Oc3ncnc(Oc4[nH]cc5c4[nH]c4cc(C=C)ccc45)c4cncc3-4)[nH]cc12] | V-984 |
| 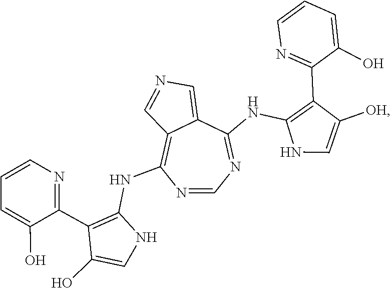 [SMILES: Oc1cccnc1-c1c(O)c[nH]c1Nc1ncnc(Nc2[nH]cc(O)c2-c2ncccc2O)c2cncc1-2] | V-985 |
| 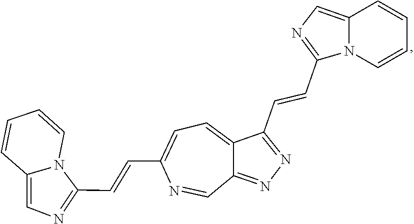 [SMILES: C(=C\c1ncc2ccccn12)/c1ccc2c(/C=C/c3ncc4ccccn34)nnc-2cn1] | V-986 |
| 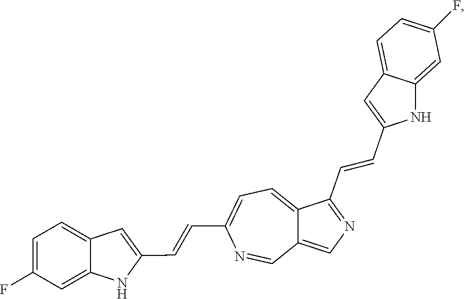 [SMILES: Fc1ccc2cc(/C=C/c3ccc4c(/C=C/c5cc6ccc(F)cc6[nH]5)ncc-4cn3)[nH]c2c1] | V-987 |
| 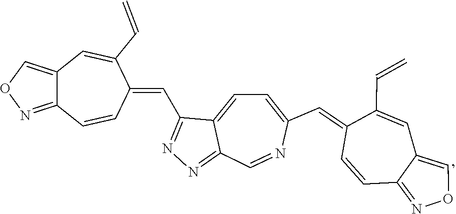 | V-988 |
| 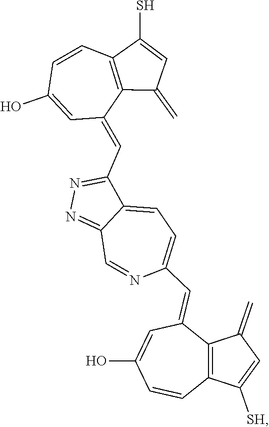 [SMILES: C=c1cc(S)c2ccc(O)c/c(=C\c3ccc4c(/C=c5\cc(O)ccc6c(S)cc(=C)c5=6)nnc-4cn3)c1=2] | V-989 |
| 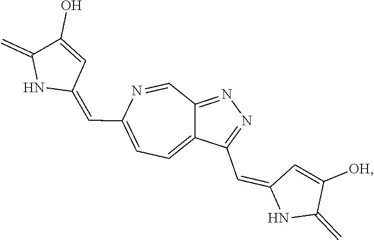 [SMILES: C=c1[nH]/c(=C/c2ccc3c(/C=c4\cc(O)c(=C)[nH]4)nnc-3cn2)cc1O] | V-990 |
| 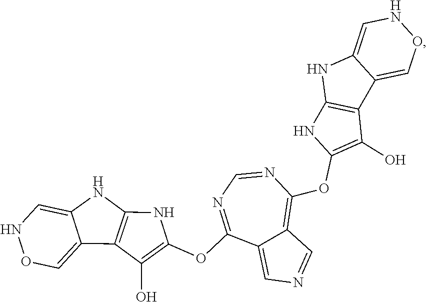 [SMILES: Oc1c(Oc2ncnc(Oc3[nH]c4[nH]c5c(c4c3O)=CONC=5)c3cncc2-3)[nH]c2[nH]c3c(c12)=CONC=3] | V-991 |
| 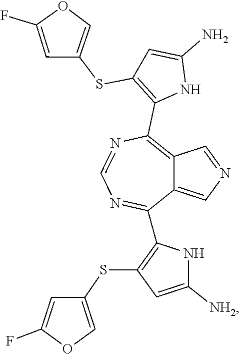 [SMILES: Nc1cc(Sc2coc(F)c2)c(-c2ncnc(-c3[nH]c(N)cc3Sc3coc(F)c3)c3cncc2-3)[nH]1] | V-992 |
| 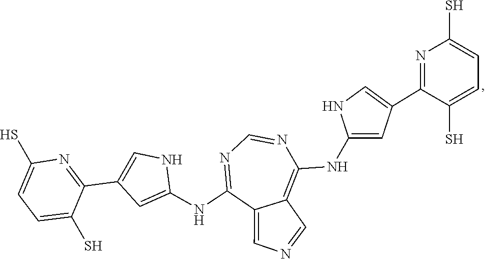 [SMILES: Sc1ccc(S)c(-c2c[nH]c(Nc3ncnc(Nc4cc(-c5nc(S)ccc5S)c[nH]4)c4cncc3-4)c2)n1] | V-993 |
| 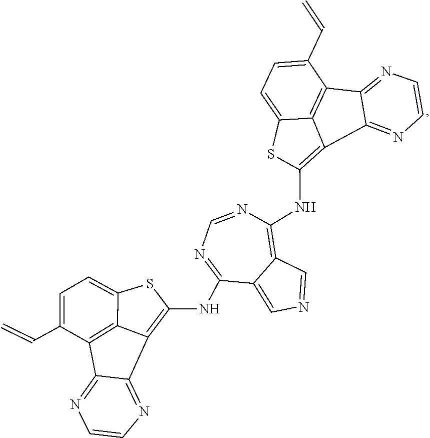 [SMILES: C=Cc1ccc2sc(Nc3ncnc(Nc4sc5ccc(C=C)c6c5c4-c4nccnc4-6)c4cncc3-4)c3c2c1-c1nccnc1-3] | V-994 |
| 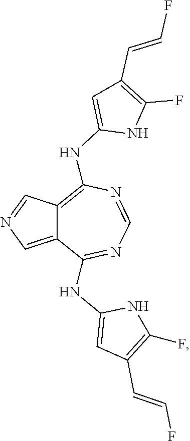 [SMILES: F/C=C/c1cc(Nc2ncnc(Nc3cc(/C=C/F)c(F)[nH]3)c3cncc2-3)[nH]c1F] | V-995 |
| 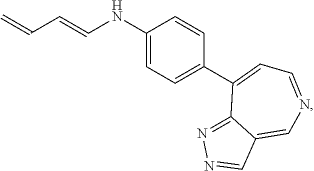 [SMILES: C=C/C=C/Nc1ccc(-c2ccncc3cnnc2-3)cc1] | V-996 |
| 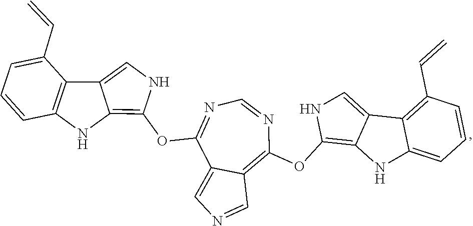 [SMILES: C=Cc1cccc2[nH]c3c(Oc4ncnc(Oc5[nH]cc6c5[nH]c5cccc(C=C)c56)c5cncc4-5)[nH]cc3c12] | V-997 |
| 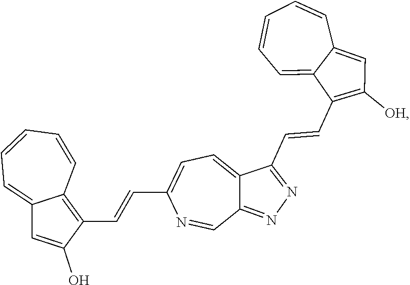 | V-998 |
| 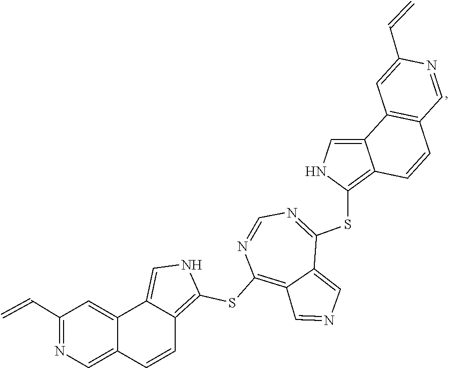 [SMILES: C=Cc1cc2c(ccc3c(Sc4ncnc(Sc5[nH]cc6c5ccc5cnc(C=C)cc56)c5cncc4-5)[nH]cc32)cn1] | V-999 |
| 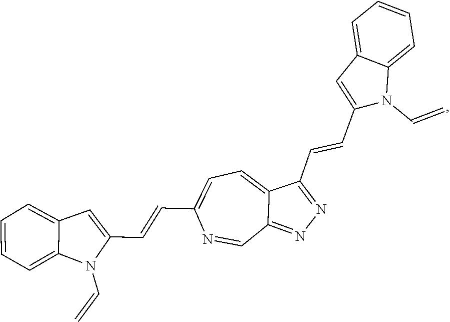 [SMILES: C=Cn1c(/C=C/c2ccc3c(/C=C/c4cc5ccccc5n4C=C)nnc-3cn2)cc2ccccc21] | V-1000 |
| 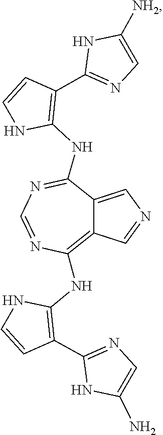 [SMILES: Nc1cnc(-c2cc[nH]c2Nc2ncnc(Nc3[nH]ccc3-c3ncc(N)[nH]3)c3cncc2-3)[nH]1] | V-1001 |
| 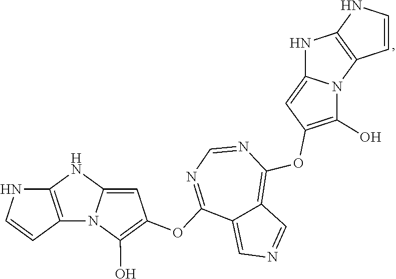 [SMILES: Oc1c(Oc2ncnc(Oc3cc4[nH]c5[nH]ccc5n4c3O)c3cncc2-3)cc2[nH]c3[nH]ccc3n12] | V-1002 |
| 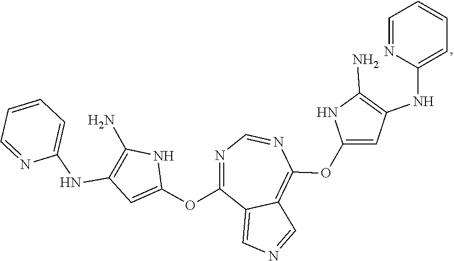 [SMILES: Nc1[nH]c(Oc2ncnc(Oc3cc(Nc4ccccn4)c(N)[nH]3)c3cncc2-3)cc1Nc1ccccn1] | V-1003 |
| 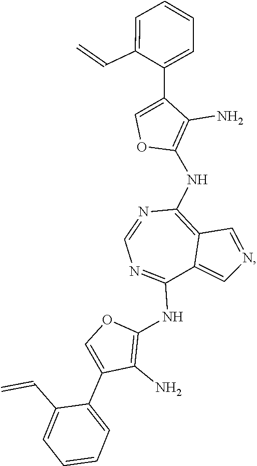 [SMILES: C=Cc1ccccc1-c1coc(Nc2ncnc(Nc3occ(-c4ccccc4C=C)c3N)c3cncc2-3)c1N] | V-1004 |
| 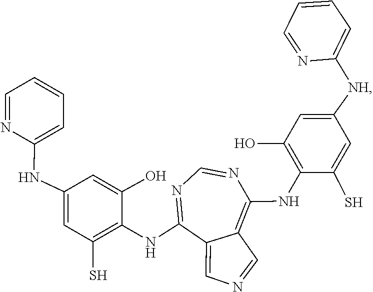 [SMILES: Oc1cc(Nc2ccccn2)cc(S)c1Nc1ncnc(Nc2c(O)cc(Nc3ccccn3)cc2S)c2cncc1-2] | V-1005 |
| 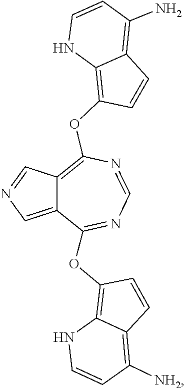 [SMILES: Nc1cc[nH]c2c(Oc3ncnc(Oc4ccc5c(N)cc[nH]c4-5)c4cncc3-4)ccc1-2] | V-1006 |
| 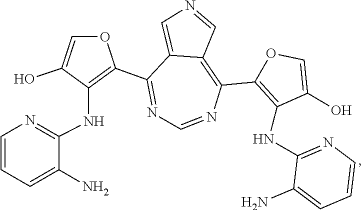 [SMILES: Nc1cccnc1Nc1c(O)coc1-c1ncnc(-c2occ(O)c2Nc2ncccc2N)c2cncc1-2] | V-1007 |
| 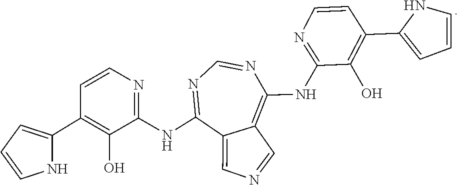 [SMILES: Oc1c(-c2ccc[nH]2)ccnc1Nc1ncnc(Nc2nccc(-c3ccc[nH]3)c2O)c2cncc1-2] | V-1008 |
| 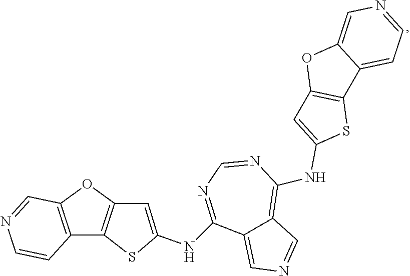 [SMILES: c1cc2c(cn1)oc1cc(Nc3ncnc(Nc4cc5oc6cnccc6c5s4)c4cncc3-4)sc12] | V-1009 |
| 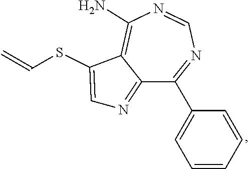 [SMILES: C=CSc1cnc2c(-c3ccccc3)ncnc(N)c1-2] | V-1010 |
| 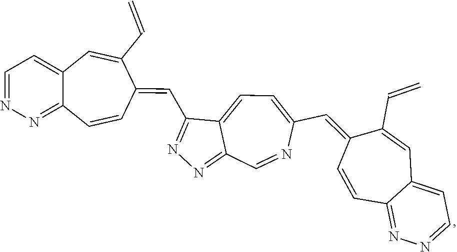 | V-1011 |
| 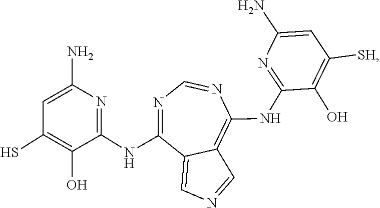 [SMILES: Nc1cc(S)c(O)c(Nc2ncnc(Nc3nc(N)cc(S)c3O)c3cncc2-3)n1] | V-1012 |
| 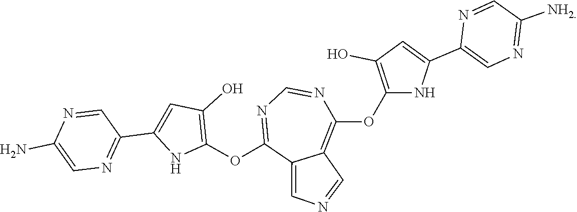 [SMILES: Nc1cnc(-c2cc(O)c(Oc3ncnc(Oc4[nH]c(-c5cnc(N)cn5)cc4O)c4cncc3-4)[nH]2)cn1] | V-1013 |
| 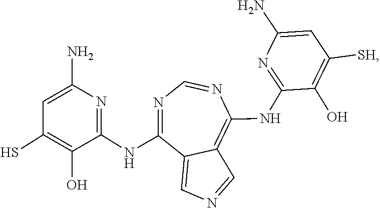 [SMILES: Nc1cc(S)c(O)c(Nc2ncnc(Nc3nc(N)cc(S)c3O)c3cncc2-3)n1] | V-1014 |
| 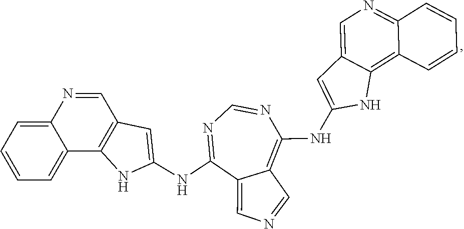 [SMILES: c1ccc2c(c1)ncc1cc(Nc3ncnc(Nc4cc5cnc6ccccc6c5[nH]4)c4cncc3-4)[nH]c12] | V-1015 |
| 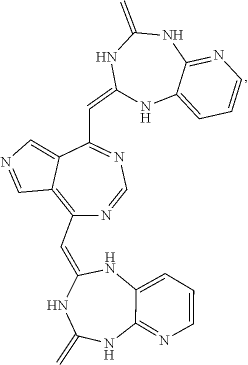 [SMILES: C=c1[nH]/c(=C/c2ncnc(/C=c3/[nH]c(=C)[nH]c4ncccc4[nH]3)c3cncc2-3)[nH]c2cccnc2[nH]1] | V-1016 |
| 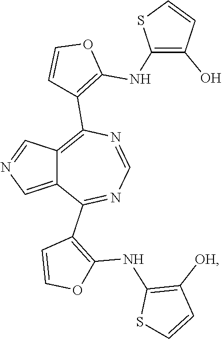 [SMILES: Oc1ccsc1Nc1occc1-c1ncnc(-c2ccoc2Nc2sccc2O)c2cncc1-2] | V-1017 |
| 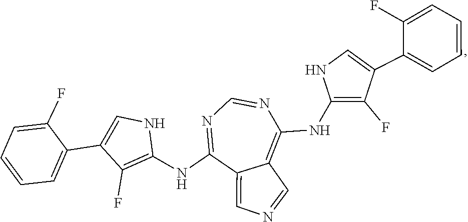 [SMILES: Fc1ccccc1-c1c[nH]c(Nc2ncnc(Nc3[nH]cc(-c4ccccc4F)c3F)c3cncc2-3)c1F] | V-1018 |
| 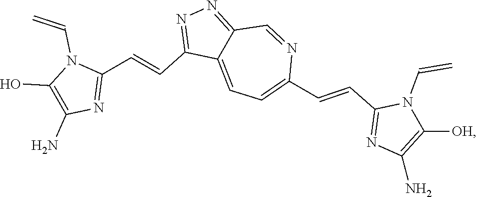 [SMILES: C=Cn1c(/C=C/c2ccc3c(/C=C/c4nc(N)c(O)n4C=C)nnc-3cn2)nc(N)c1O] | V-1019 |
| 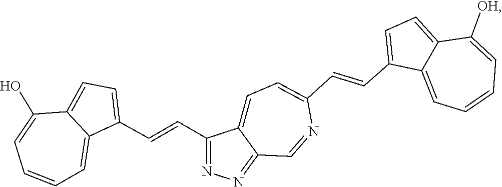 [SMILES: Oc1ccccc2c(/C=C/c3ccc4c(/C=C/c5ccc6c(O)ccccc5-6)nnc-4cn3)ccc1-2] | V-1020 |
| 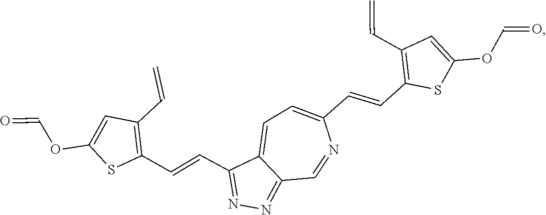 [SMILES: C=Cc1cc(OC=O)sc1/C=C/c1ccc2c(/C=C/c3sc(OC=O)cc3C=C)nnc-2cn1] | V-1021 |
| 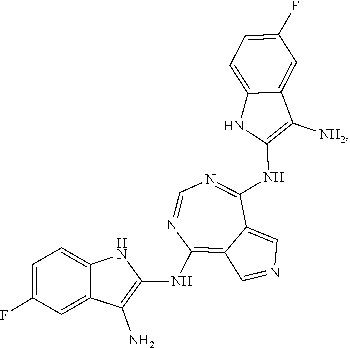 [SMILES: Nc1c(Nc2ncnc(Nc3[nH]c4ccc(F)cc4c3N)c3cncc2-3)[nH]c2ccc(F)cc12] | V-1022 |
| 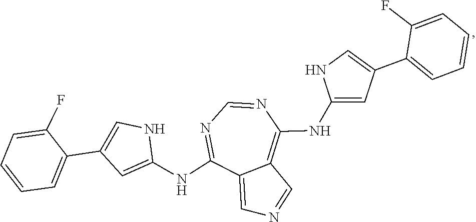 [SMILES: Fc1ccccc1-c1c[nH]c(Nc2ncnc(Nc3cc(-c4ccccc4F)c[nH]3)c3cncc2-3)c1] | V-1023 |
| 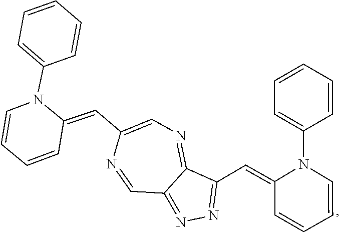 [SMILES: C1=C/C(=C\c2cnc3c(/C=C4\C=CC=CN4c4ccccc4)nnc-3cn2)N(c2ccccc2)C=C1] | V-1024 |
| 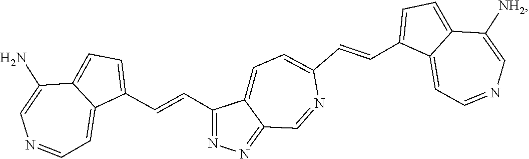 [SMILES: Nc1cnccc2c(/C=C/c3ccc4c(/C=C/c5ccc6c(N)cnccc5-6)nnc-4cn3)ccc1-2] | V-1025 |
| 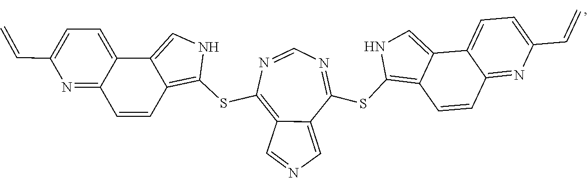 [SMILES: C=Cc1ccc2c(ccc3c(Sc4ncnc(Sc5[nH]cc6c5ccc5nc(C=C)ccc56)c5cncc4-5)[nH]cc32)n1] | V-1026 |
| 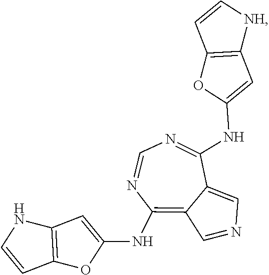 [SMILES: c1nc(Nc2cc3[nH]ccc3o2)c2cncc-2c(Nc2cc3[nH]ccc3o2)n1] | V-1027 |
| 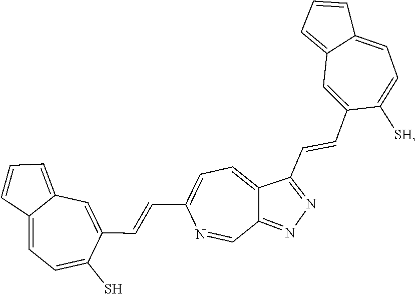 | V-1028 |
| 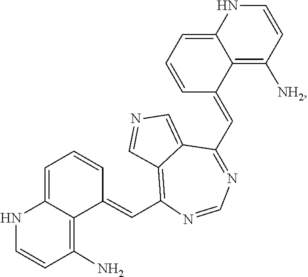 [SMILES: NC1=c2c(ccc/c2=C\c2ncnc(/C=c3\cccc4c3=C(N)C=CN4)c3cncc2-3)NC=C1] | V-1029 |
| 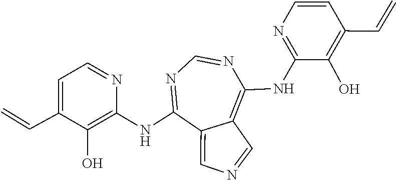 [SMILES: C=Cc1ccnc(Nc2ncnc(Nc3nccc(C=C)c3O)c3cncc2-3)c1O] | V-1030 |
| 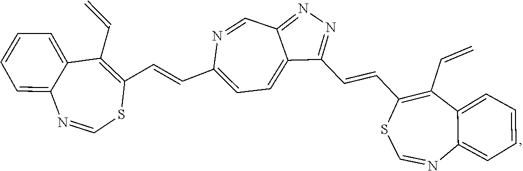 [SMILES: C=CC1=C(/C=C/c2ccc3c(/C=C/C4=C(C=C)c5ccccc5N=CS4)nnc-3cn2)SC=Nc2ccccc21] | V-1031 |
| 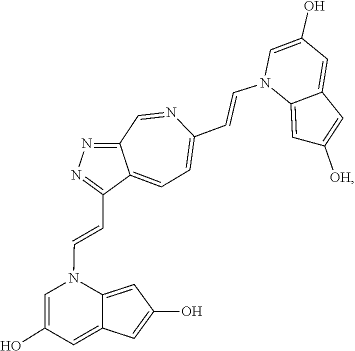 [SMILES: Oc1cc2cc(O)cn(/C=C/c3ccc4c(/C=C/n5cc(O)cc6cc(O)cc5-6)nnc-4cn3)c-2c1] | V-1032 |
| 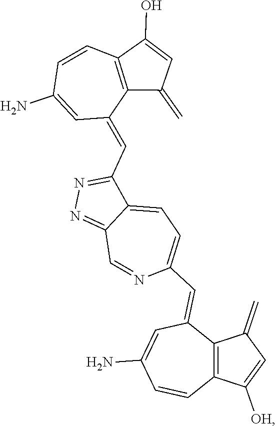 [SMILES: C=c1cc(O)c2ccc(N)c/c(=C\c3ccc4c(/C=c5\cc(N)ccc6c(O)cc(=C)c5=6)nnc-4cn3)c1=2] | V-1033 |
| 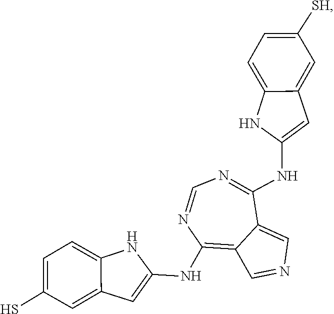 [SMILES: Sc1ccc2[nH]c(Nc3ncnc(Nc4cc5cc(S)ccc5[nH]4)c4cncc3-4)cc2c1] | V-1034 |
| 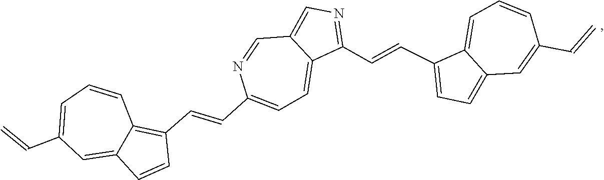 [SMILES: C=Cc1cccc2c(/C=C/c3ccc4c(/C=C/c5ccc6cc(C=C)cccc5-6)ncc-4cn3)ccc-2c1] | V-1035 |
| 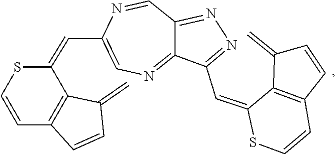 | V-1036 |
| 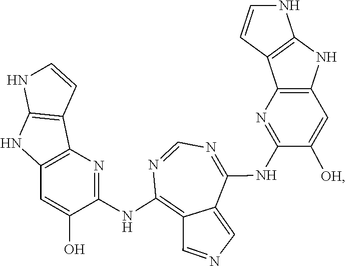 [SMILES: Oc1cc2[nH]c3[nH]ccc3c2nc1Nc1ncnc(Nc2nc3c(cc2O)[nH]c2[nH]ccc23)c2cncc1-2] | V-1037 |
| 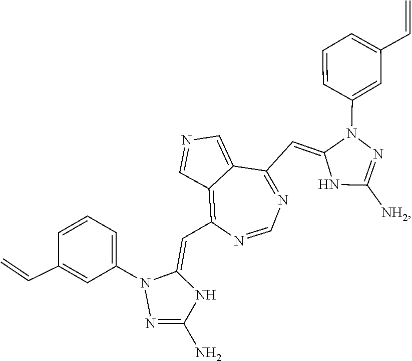 [SMILES: C=Cc1cccc(N2N=C(N)N/C2=C\c2ncnc(/C=C3\NC(N)=NN3c3cccc(C=C)c3)c3cncc2-3)c1] | V-1038 |
| 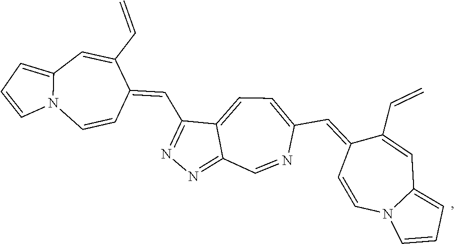 | V-1039 |
| 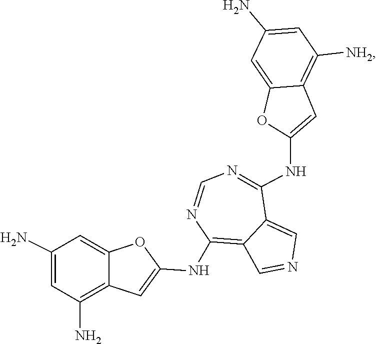 [SMILES: Nc1cc(N)c2cc(Nc3ncnc(Nc4cc5c(N)cc(N)cc5o4)c4cncc3-4)oc2c1] | V-1040 |
| 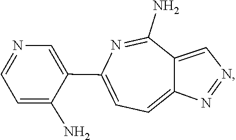 | V-1041 |
| 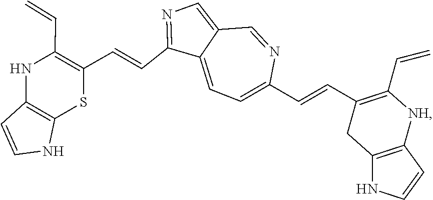 [SMILES: C=CC1=C(/C=C/c2ccc3c(/C=C/C4=C(C=C)Nc5cc[nH]c5S4)ncc-3cn2)Cc2[nH]ccc2N1] | V-1042 |
| 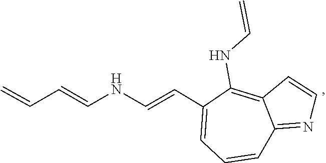 [SMILES: C=C/C=C/N/C=C/c1cccc2nccc-2c1NC=C] | V-1043 |
| 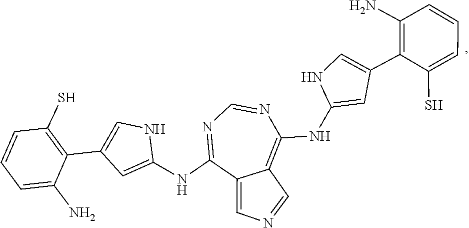 [SMILES: Nc1cccc(S)c1-c1c[nH]c(Nc2ncnc(Nc3cc(-c4c(N)cccc4S)c[nH]3)c3cncc2-3)c1] | V-1044 |
| 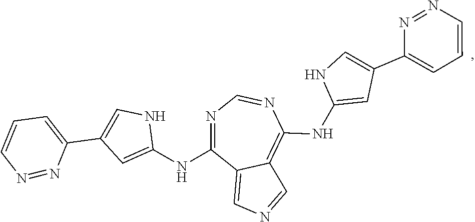 [SMILES: c1cnnc(-c2c[nH]c(Nc3ncnc(Nc4cc(-c5cccnn5)c[nH]4)c4cncc3-4)c2)c1] | V-1045 |
| 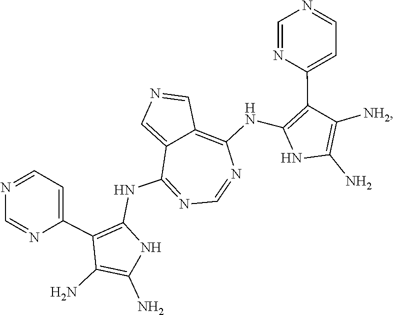 [SMILES: Nc1[nH]c(Nc2ncnc(Nc3[nH]c(N)c(N)c3-c3ccncn3)c3cncc2-3)c(-c2ccncn2)c1N] | V-1046 |
| 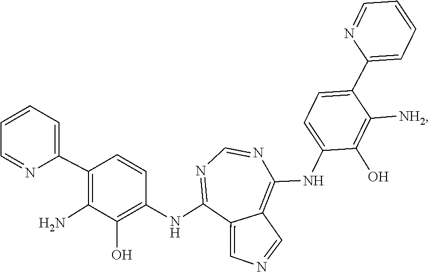 [SMILES: Nc1c(-c2ccccn2)ccc(Nc2ncnc(Nc3ccc(-c4ccccn4)c(N)c3O)c3cncc2-3)c1O] | V-1047 |
| 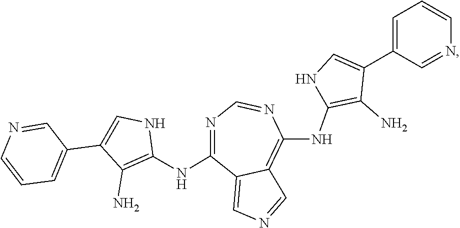 [SMILES: Nc1c(-c2cccnc2)c[nH]c1Nc1ncnc(Nc2[nH]cc(-c3cccnc3)c2N)c2cncc1-2] | V-1048 |
| 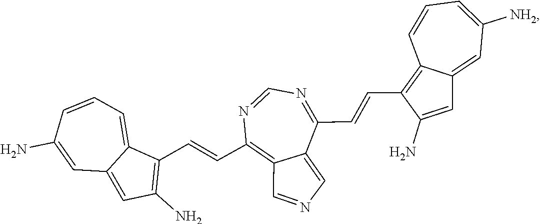 [SMILES: Nc1cccc2c(/C=C/c3ncnc(/C=C/c4c(N)cc5cc(N)cccc4-5)c4cncc3-4)c(N)cc-2c1] | V-1049 |
| 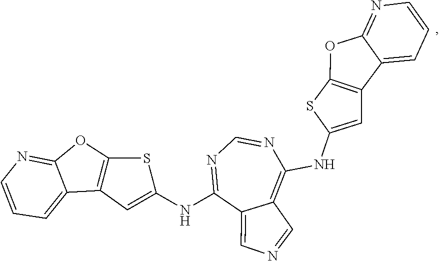 [SMILES: c1cnc2oc3sc(Nc4ncnc(Nc5cc6c(oc7ncccc76)s5)c5cncc4-5)cc3c2c1] | V-1050 |
| 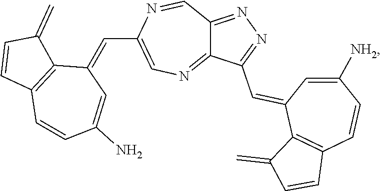 [SMILES: C=c1ccc2ccc(N)c/c(=C\c3cnc4c(/C=c5\cc(N)ccc6ccc(=C)c5=6)nnc-4cn3)c1=2] | V-1051 |
| 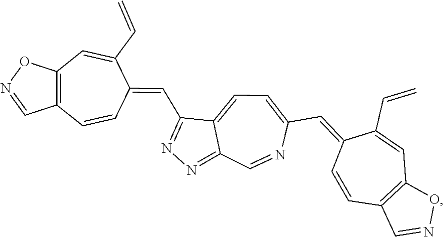 [SMILES: C=CC1=Cc2oncc2C=C/C1=C\c1ccc2c(/C=C3\C=Cc4cnoc4C=C3C=C)nnc-2cn1] | V-1052 |
| 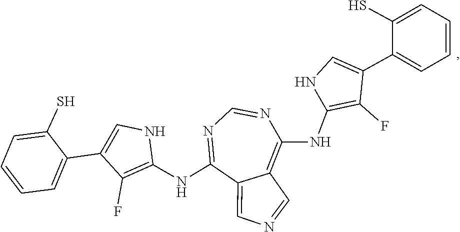 [SMILES: Fc1c(-c2ccccc2S)c[nH]c1Nc1ncnc(Nc2[nH]cc(-c3ccccc3S)c2F)c2cncc1-2] | V-1053 |
| 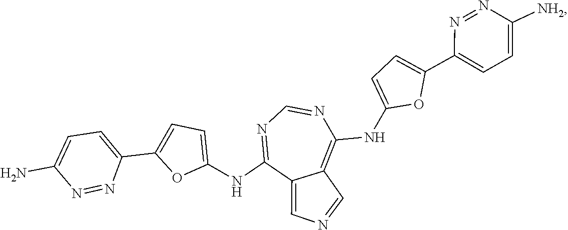 [SMILES: Nc1ccc(-c2ccc(Nc3ncnc(Nc4ccc(-c5ccc(N)nn5)o4)c4cncc3-4)o2)nn1] | V-1054 |
| 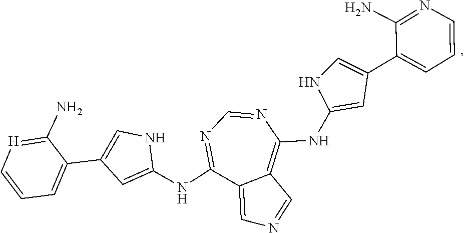 | V-1055 |
| 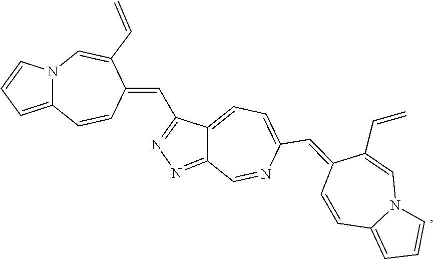 | V-1056 |
| 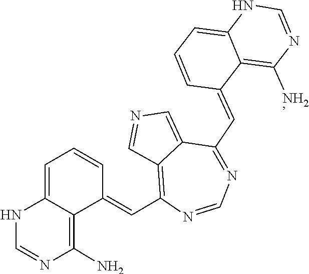 [SMILES: NC1=c2c(ccc/c2=C\c2ncnc(/C=c3\cccc4c3=C(N)N=CN4)c3cncc2-3)NC=N1] | V-1057 |
| 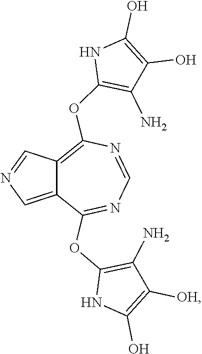 [SMILES: Nc1c(Oc2ncnc(Oc3[nH]c(O)c(O)c3N)c3cncc2-3)[nH]c(O)c1O] | V-1058 |
| 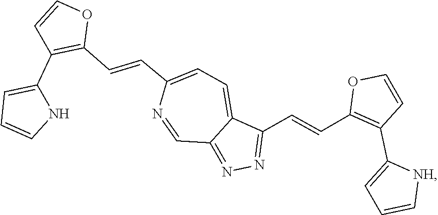 [SMILES: C(=C\c1occc1-c1ccc[nH]1)/c1ccc2c(/C=C/c3occc3-c3ccc[nH]3)nnc-2cn1] | V-1059 |
| 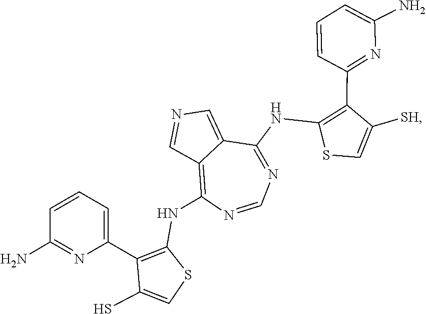 [SMILES: Nc1cccc(-c2c(S)csc2Nc2ncnc(Nc3scc(S)c3-c3cccc(N)n3)c3cncc2-3)n1] | V-1060 |
| 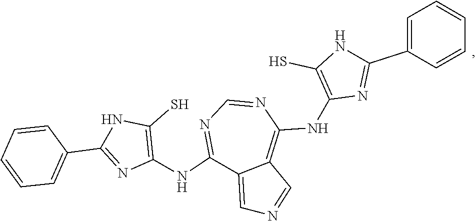 [SMILES: Sc1[nH]c(-c2ccccc2)nc1Nc1ncnc(Nc2nc(-c3ccccc3)[nH]c2S)c2cncc1-2] | V-1061 |
| 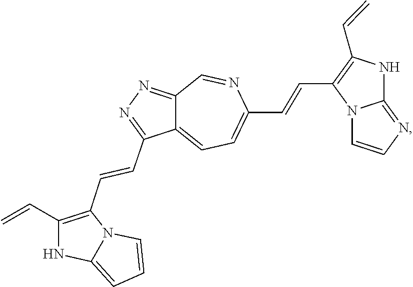 [SMILES: C=Cc1[nH]c2cccn2c1/C=C/c1nnc2cnc(/C=C/c3c(C=C)[nH]c4nccn34)ccc1-2] | V-1062 |
| 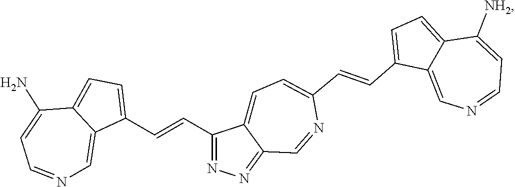 [SMILES: Nc1ccncc2c(/C=C/c3ccc4c(/C=C/c5ccc6c(N)ccncc5-6)nnc-4cn3)ccc1-2] | V-1063 |
| 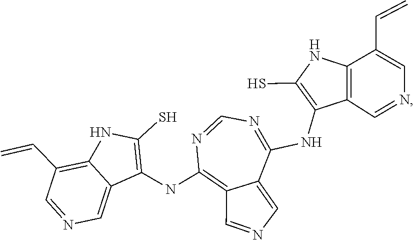 | V-1064 |
| 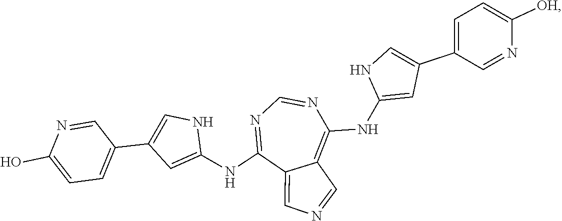 [SMILES: Oc1ccc(-c2c[nH]c(Nc3ncnc(Nc4cc(-c5ccc(O)nc5)c[nH]4)c4cncc3-4)c2)cn1] | V-1065 |
| 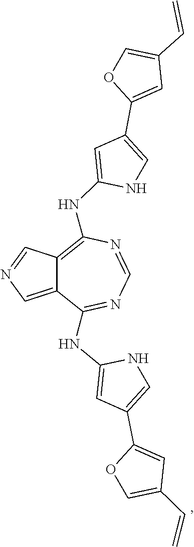 [SMILES: C=Cc1coc(-c2c[nH]c(Nc3ncnc(Nc4cc(-c5cc(C=C)co5)c[nH]4)c4cncc3-4)c2)c1] | V-1066 |
| 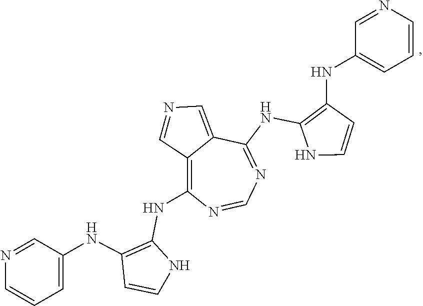 [SMILES: c1cncc(Nc2cc[nH]c2Nc2ncnc(Nc3[nH]ccc3Nc3cccnc3)c3cncc2-3)c1] | V-1067 |
| 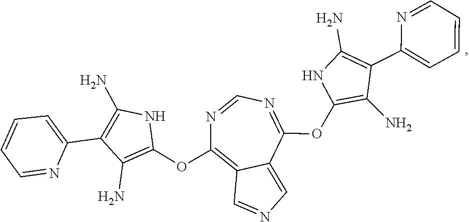 [SMILES: Nc1[nH]c(Oc2ncnc(Oc3[nH]c(N)c(-c4ccccn4)c3N)c3cncc2-3)c(N)c1-c1ccccn1] | V-1068 |
| 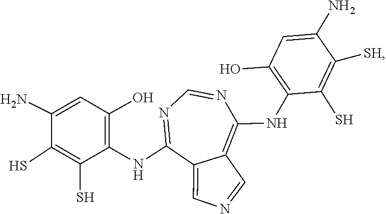 [SMILES: Nc1cc(O)c(Nc2ncnc(Nc3c(O)cc(N)c(S)c3S)c3cncc2-3)c(S)c1S] | V-1069 |
| 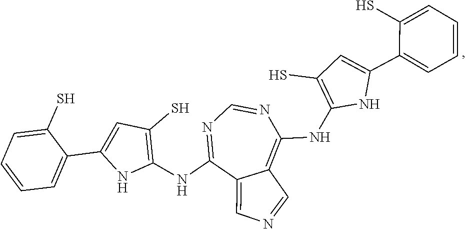 [SMILES: Sc1ccccc1-c1cc(S)c(Nc2ncnc(Nc3[nH]c(-c4ccccc4S)cc3S)c3cncc2-3)[nH]1] | V-1070 |
| 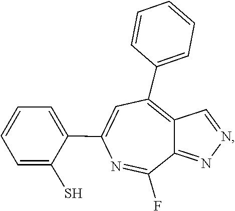 [SMILES: Fc1nc(-c2ccccc2S)cc(-c2ccccc2)c2cnnc1-2] | V-1071 |
| 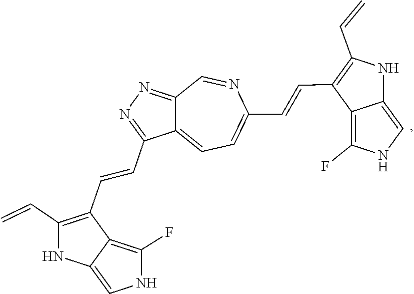 [SMILES: C=Cc1[nH]c2c[nH]c(F)c2c1/C=C/c1ccc2c(/C=C/c3c(C=C)[nH]c4c[nH]c(F)c34)nnc-2cn1] | V-1072 |
| 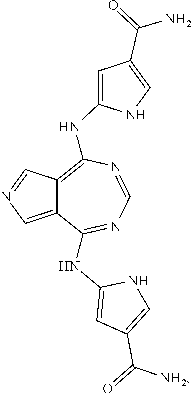 [SMILES: NC(=O)c1c[nH]c(Nc2ncnc(Nc3cc(C(N)=O)c[nH]3)c3cncc2-3)c1] | V-1073 |
| 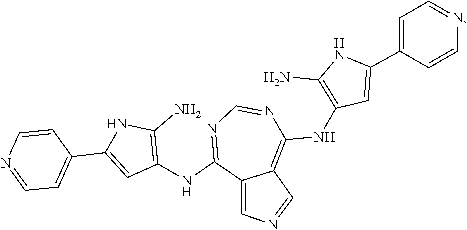 [SMILES: Nc1[nH]c(-c2ccncc2)cc1Nc1ncnc(Nc2cc(-c3ccncc3)[nH]c2N)c2cncc1-2] | V-1074 |
| 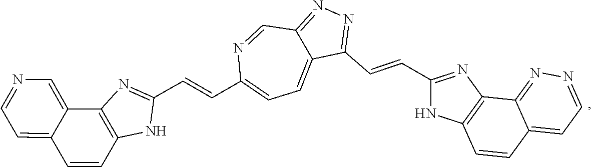 [SMILES: C(=C\c1nc2c(ccc3ccncc32)[nH]1)/c1ccc2c(/C=C/c3nc4c(ccc5ccnnc54)[nH]3)nnc-2cn1] | V-1075 |
| 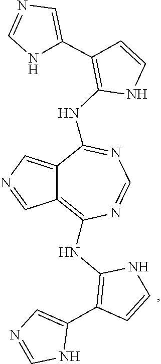 [SMILES: c1nc(Nc2[nH]ccc2-c2cnc[nH]2)c2cncc-2c(Nc2[nH]ccc2-c2cnc[nH]2)n1] | V-1076 |
| 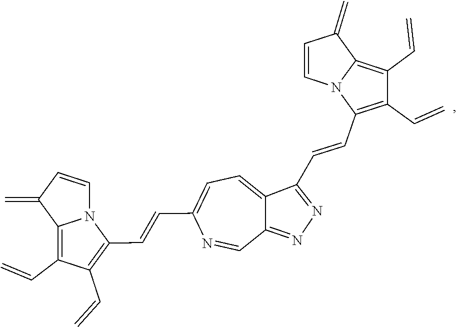 | V-1077 |
| 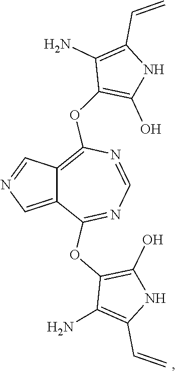 [SMILES: C=Cc1[nH]c(O)c(Oc2ncnc(Oc3c(O)[nH]c(C=C)c3N)c3cncc2-3)c1N] | V-1078 |
| 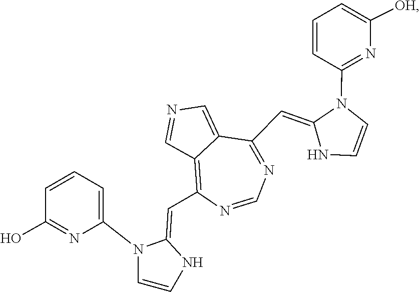 [SMILES: Oc1cccc(N2C=CN/C2=C\c2ncnc(/C=C3\NC=CN3c3cccc(O)n3)c3cncc2-3)n1] | V-1079 |
| 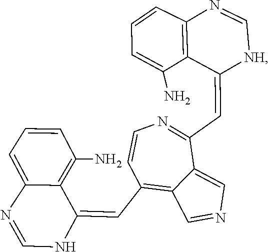 [SMILES: Nc1cccc2c1/C(=C\c1ccnc(/C=C3/NC=Nc4cccc(N)c43)c3cncc1-3)NC=N2] | V-1080 |
| 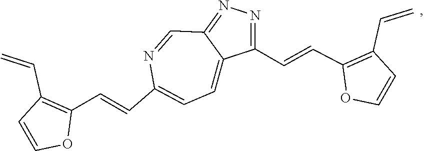 [SMILES: C=Cc1ccoc1/C=C/c1ccc2c(/C=C/c3occc3C=C)nnc-2cn1] | V-1081 |
| 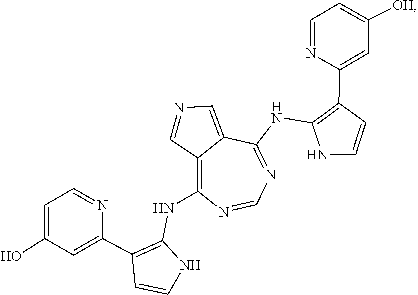 [SMILES: Oc1ccnc(-c2cc[nH]c2Nc2ncnc(Nc3[nH]ccc3-c3cc(O)ccn3)c3cncc2-3)c1] | V-1082 |
| 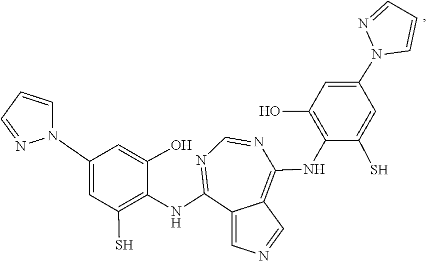 [SMILES: Oc1cc(-n2cccn2)cc(S)c1Nc1ncnc(Nc2c(O)cc(-n3cccn3)cc2S)c2cncc1-2] | V-1083 |
| 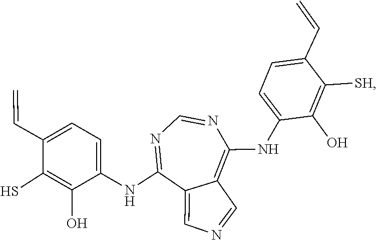 [SMILES: C=Cc1ccc(Nc2ncnc(Nc3ccc(C=C)c(S)c3O)c3cncc2-3)c(O)c1S] | V-1084 |
| 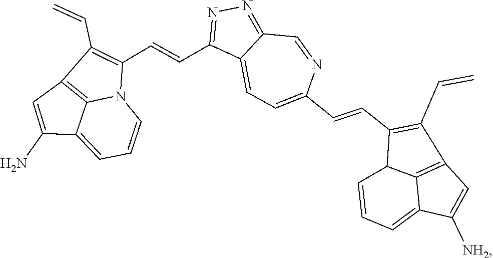 [SMILES: C=CC1=C(/C=C/c2ccc3c(/C=C/c4c(C=C)c5cc(N)c6cccn4c65)nnc-3cn2)C2C=CC=C3C(N)=CC1=C32] | V-1085 |
| 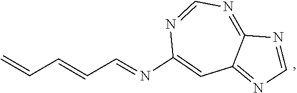 [SMILES: C=C/C=C/C=N/c1cc2ncnc-2ncn1] | V-1086 |
| 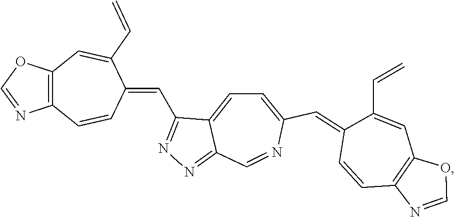 | V-1087 |
| 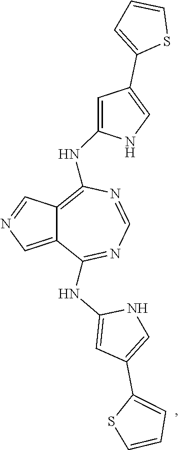 [SMILES: c1csc(-c2c[nH]c(Nc3ncnc(Nc4cc(-c5cccs5)c[nH]4)c4cncc3-4)c2)c1] | V-1088 |
| 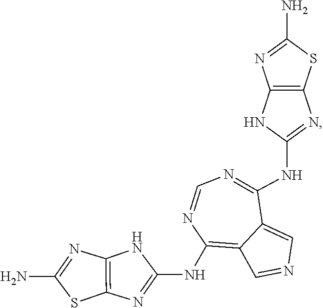 [SMILES: Nc1nc2[nH]c(Nc3ncnc(Nc4nc5sc(N)nc5[nH]4)c4cncc3-4)nc2s1] | V-1089 |
| 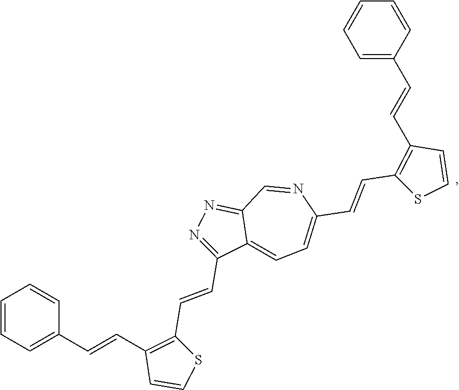 [SMILES: C(=C\c1ccsc1/C=C/c1ccc2c(/C=C/c3sccc3/C=C/c3ccccc3)nnc-2cn1)/c1ccccc1] | V-1090 |
| 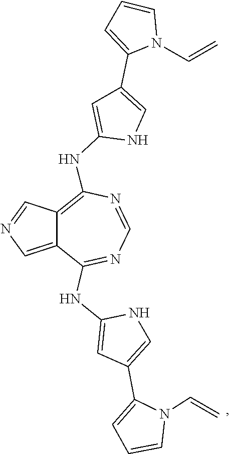 [SMILES: C=Cn1cccc1-c1c[nH]c(Nc2ncnc(Nc3cc(-c4cccn4C=C)c[nH]3)c3cncc2-3)c1] | V-1091 |
| 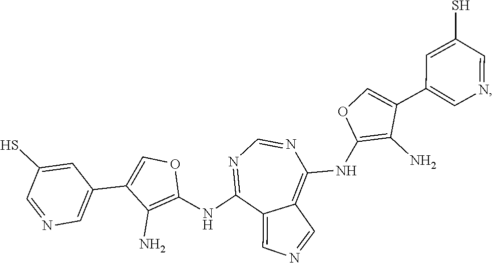 [SMILES: Nc1c(-c2cncc(S)c2)coc1Nc1ncnc(Nc2occ(-c3cncc(S)c3)c2N)c2cncc1-2] | V-1092 |
| 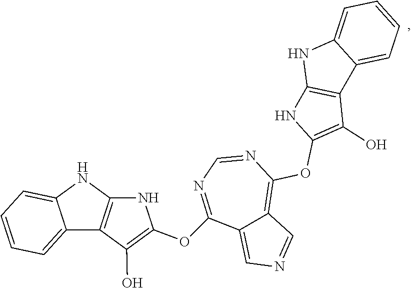 [SMILES: Oc1c(Oc2ncnc(Oc3[nH]c4[nH]c5ccccc5c4c3O)c3cncc2-3)[nH]c2[nH]c3ccccc3c12] | V-1093 |
| 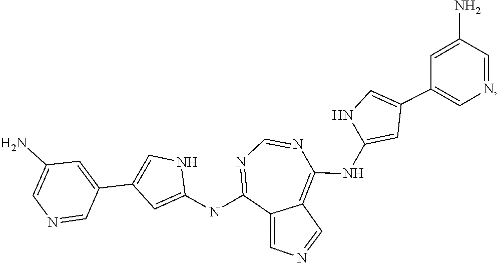 | V-1094 |
| 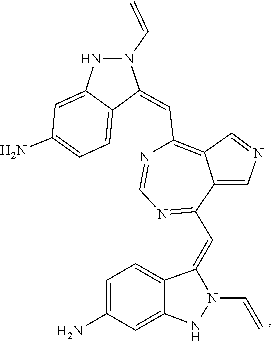 | V-1095 |
| 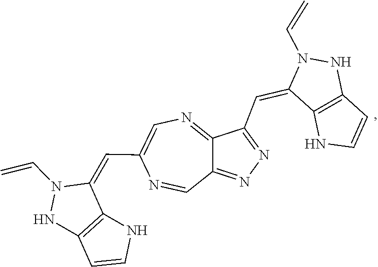 [SMILES: C=CN1Nc2cc[nH]c2/C1=C\c1cnc2c(/C=C3\c4[nH]ccc4NN3C=C)nnc-2cn1] | V-1096 |
| 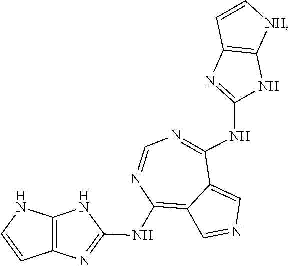 [SMILES: c1nc(Nc2nc3cc[nH]c3[nH]2)c2cncc-2c(Nc2nc3cc[nH]c3[nH]2)n1] | V-1097 |
| 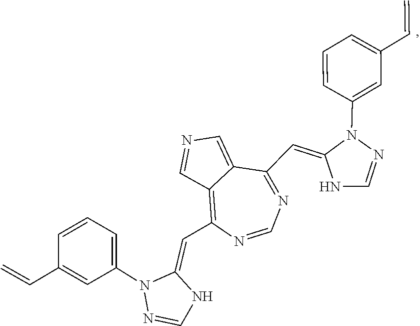 [SMILES: C=Cc1cccc(N2N=CN/C2=C\c2ncnc(/C=C3\NC=NN3c3cccc(C=C)c3)c3cncc2-3)c1] | V-1098 |
| 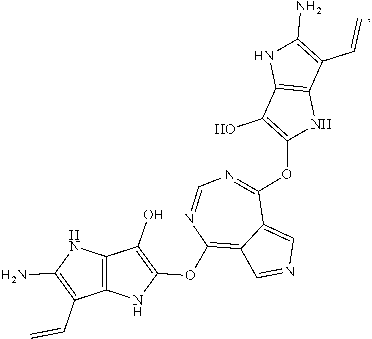 [SMILES: C=Cc1c(N)[nH]c2c(O)c(Oc3ncnc(Oc4[nH]c5c(C=C)c(N)[nH]c5c4O)c4cncc3-4)[nH]c12] | V-1099 |
| 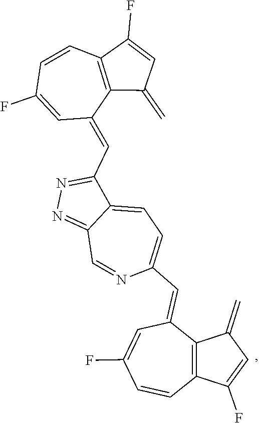 [SMILES: C=c1cc(F)c2ccc(F)c/c(=C\c3ccc4c(/C=c5\cc(F)ccc6c(F)cc(=C)c5=6)nnc-4cn3)c1=2] | V-1100 |
| 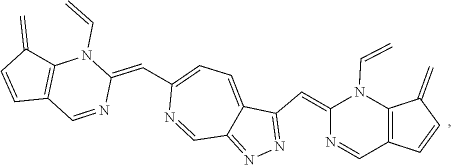 [SMILES: C=Cn1c2c(=C)ccc=2cn/c1=C\c1ccc2c(/C=c3\ncc4ccc(=C)c=4n3C=C)nnc-2cn1] | V-1101 |
| 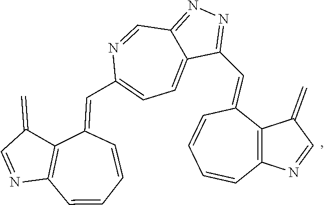 [SMILES: C=c1cnc2cccc/c(=C\c3ccc4c(/C=c5\ccccc6ncc(=C)c5=6)nnc-4cn3)c1=2] | V-1102 |
| 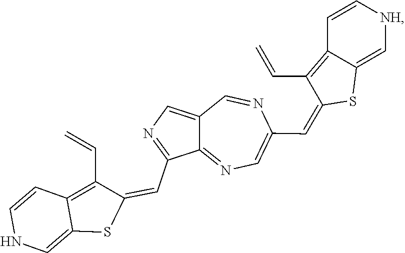 [SMILES: C=Cc1c2c(s/c1=C/c1cnc3c(/C=c4/sc5c(c4C=C)C=CNC=5)ncc-3cn1)=CNC=C2] | V-1103 |
| 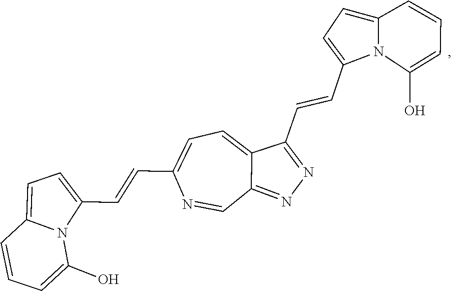 [SMILES: Oc1cccc2ccc(/C=C/c3ccc4c(/C=C/c5ccc6cccc(O)n56)nnc-4cn3)n12] | V-1104 |
| 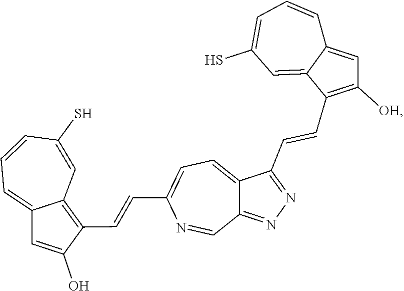 [SMILES: Oc1cc2cccc(S)cc-2c1/C=C/c1ccc2c(/C=C/c3c(O)cc4cccc(S)cc3-4)nnc-2cn1] | V-1105 |
| 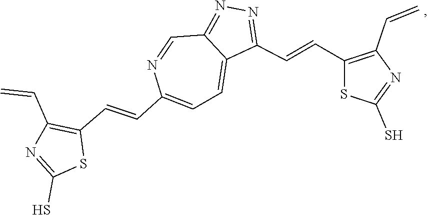 [SMILES: C=Cc1nc(S)sc1/C=C/c1ccc2c(/C=C/c3sc(S)nc3C=C)nnc-2cn1] | V-1106 |
| 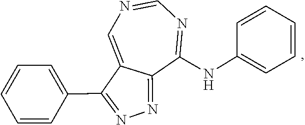 [SMILES: c1ccc(Nc2ncncc3c(-c4ccccc4)nnc2-3)cc1] | V-1107 |
| 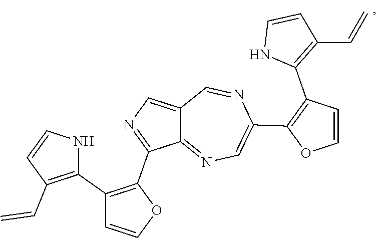 [SMILES: C=Cc1cc[nH]c1-c1ccoc1-c1cnc2c(-c3occc3-c3[nH]ccc3C=C)ncc-2cn1] | V-1108 |
| 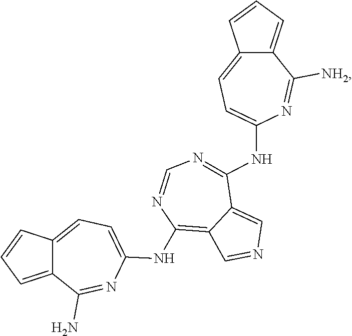 [SMILES: Nc1nc(Nc2ncnc(Nc3ccc4cccc-4c(N)n3)c3cncc2-3)ccc2cccc1-2] | V-1109 |
| 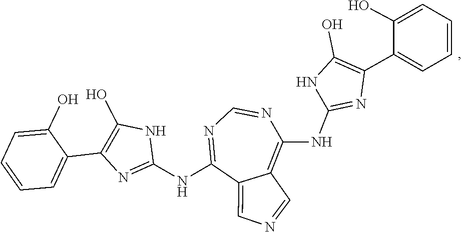 [SMILES: Oc1ccccc1-c1nc(Nc2ncnc(Nc3nc(-c4ccccc4O)c(O)[nH]3)c3cncc2-3)[nH]c1O] | V-1110 |
| 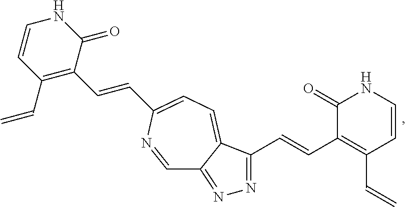 [SMILES: C=Cc1cc[nH]c(=O)c1/C=C/c1ccc2c(/C=C/c3c(C=C)cc[nH]c3=O)nnc-2cn1] | V-1111 |
| 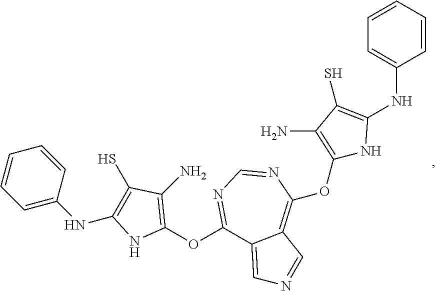 [SMILES: Nc1c(Oc2ncnc(Oc3[nH]c(Nc4ccccc4)c(S)c3N)c3cncc2-3)[nH]c(Nc2ccccc2)c1S] | V-1112 |
| 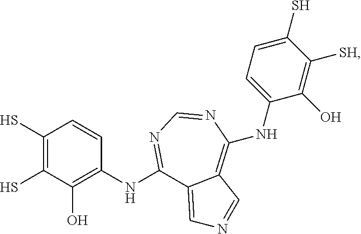 [SMILES: Oc1c(Nc2ncnc(Nc3ccc(S)c(S)c3O)c3cncc2-3)ccc(S)c1S] | V-1113 |
| 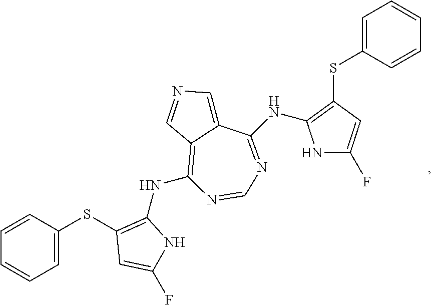 [SMILES: Fc1cc(Sc2ccccc2)c(Nc2ncnc(Nc3[nH]c(F)cc3Sc3ccccc3)c3cncc2-3)[nH]1] | V-1114 |
| 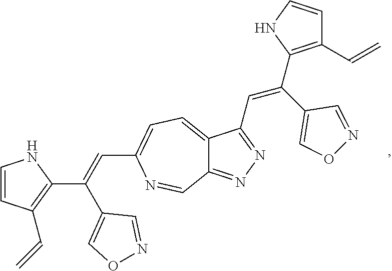 [SMILES: C=Cc1cc[nH]c1/C(=C/c1ccc2c(/C=C(\c3cnoc3)c3[nH]ccc3C=C)nnc-2cn1)c1cnoc1] | V-1115 |
| 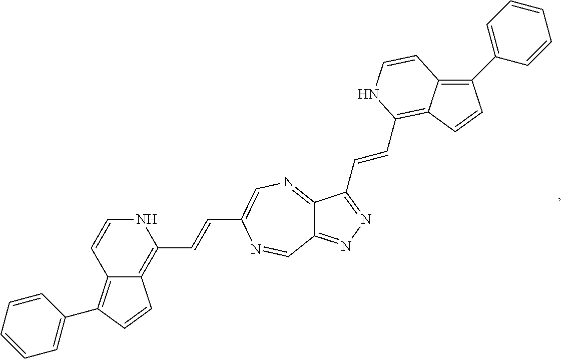 [SMILES: C(=C\c1[nH]ccc2c(-c3ccccc3)ccc1-2)/c1cnc2c(/C=C/c3[nH]ccc4c(-c5ccccc5)ccc3-4)nnc-2cn1] | V-1116 |
| 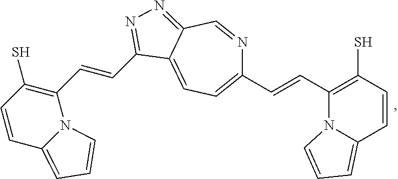 [SMILES: Sc1ccc2cccn2c1/C=C/c1ccc2c(/C=C/c3c(S)ccc4cccn34)nnc-2cn1] | V-1117 |
| 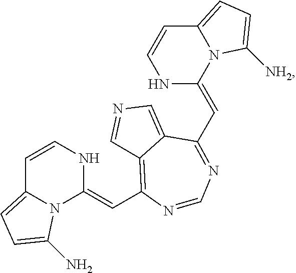 [SMILES: Nc1ccc2n1/C(=C/c1ncnc(/C=C3\NC=Cc4ccc(N)n43)c3cncc1-3)NC=C2] | V-1118 |
| 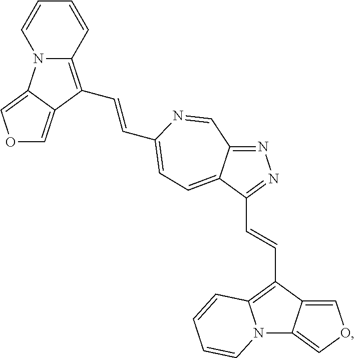 [SMILES: C(=C\c1c2cocc2n2ccccc12)/c1ccc2c(/C=C/c3c4cocc4n4ccccc34)nnc-2cn1] | V-1119 |
| 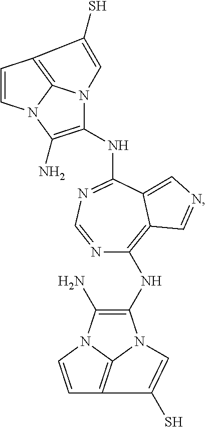 [SMILES: Nc1c(Nc2ncnc(Nc3c(N)n4ccc5c(S)cn3c54)c3cncc2-3)n2cc(S)c3ccn1c32] | V-1120 |
| 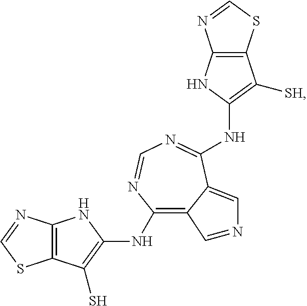 [SMILES: Sc1c(Nc2ncnc(Nc3[nH]c4ncsc4c3S)c3cncc2-3)[nH]c2ncsc12] | V-1121 |
| 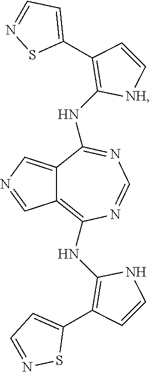 [SMILES: c1cc(-c2cc[nH]c2Nc2ncnc(Nc3[nH]ccc3-c3ccns3)c3cncc2-3)sn1] | V-1122 |
| 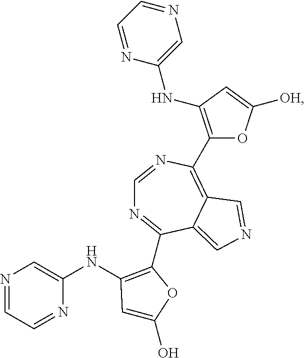 [SMILES: Oc1cc(Nc2cnccn2)c(-c2ncnc(-c3oc(O)cc3Nc3cnccn3)c3cncc2-3)o1] | V-1123 |
| 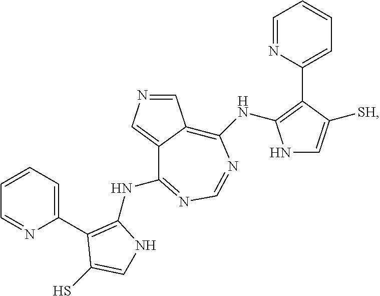 [SMILES: Sc1c[nH]c(Nc2ncnc(Nc3[nH]cc(S)c3-c3ccccn3)c3cncc2-3)c1-c1ccccn1] | V-1124 |
| 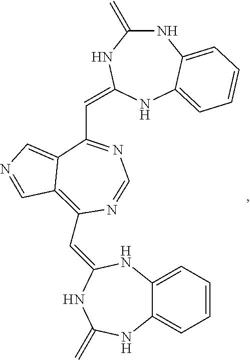 [SMILES: C=c1[nH]/c(=C/c2ncnc(/C=c3/[nH]c(=C)[nH]c4ccccc4[nH]3)c3cncc2-3)[nH]c2ccccc2[nH]1] | V-1125 |
| 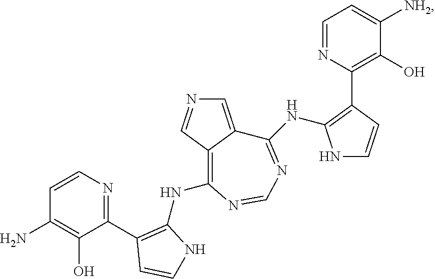 [SMILES: Nc1ccnc(-c2cc[nH]c2Nc2ncnc(Nc3[nH]ccc3-c3nccc(N)c3O)c3cncc2-3)c1O] | V-1126 |
| 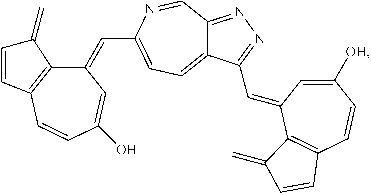 [SMILES: C=c1ccc2ccc(O)c/c(=C\c3ccc4c(/C=c5\cc(O)ccc6ccc(=C)c5=6)nnc-4cn3)c1=2] | V-1127 |
| 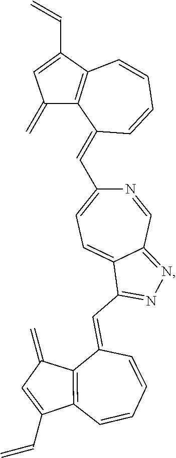 [SMILES: C=Cc1cc(=C)c2/c(=C/c3ccc4c(/C=c5\ccccc6c(C=C)cc(=C)c5=6)nnc-4cn3)ccccc1=2] | V-1128 |
| 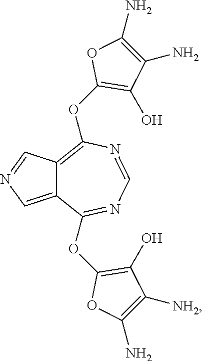 [SMILES: Nc1oc(Oc2ncnc(Oc3oc(N)c(N)c3O)c3cncc2-3)c(O)c1N] | V-1129 |
| 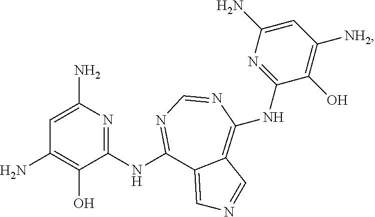 [SMILES: Nc1cc(N)c(O)c(Nc2ncnc(Nc3nc(N)cc(N)c3O)c3cncc2-3)n1] | V-1130 |
| 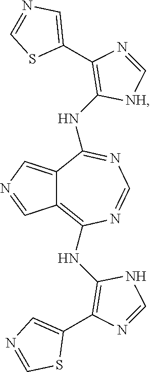 [SMILES: c1nc(Nc2[nH]cnc2-c2cncs2)c2cncc-2c(Nc2[nH]cnc2-c2cncs2)n1] | V-1131 |
| 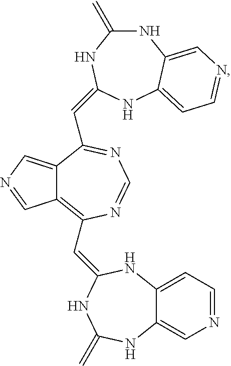 [SMILES: C=c1[nH]/c(=C/c2ncnc(/C=c3/[nH]c(=C)[nH]c4cnccc4[nH]3)c3cncc2-3)[nH]c2ccncc2[nH]1] | V-1132 |
| 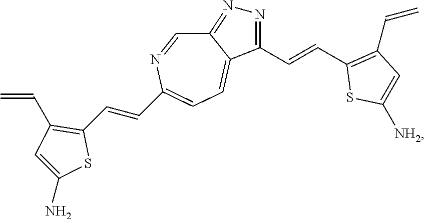 [SMILES: C=Cc1cc(N)sc1/C=C/c1ccc2c(/C=C/c3sc(N)cc3C=C)nnc-2cn1] | V-1133 |
| 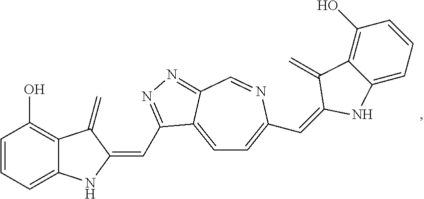 [SMILES: C=c1/c(=C\c2ccc3c(/C=c4/[nH]c5cccc(O)c5c4=C)nnc-3cn2)[nH]c2cccc(O)c12] | V-1134 |
| 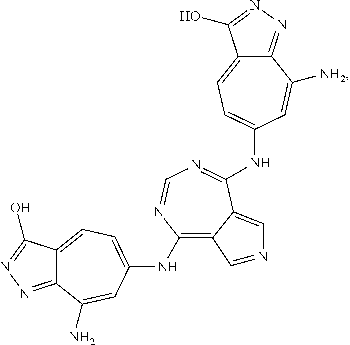 [SMILES: Nc1cc(Nc2ncnc(Nc3ccc4c(O)nnc-4c(N)c3)c3cncc2-3)ccc2c(O)nnc1-2] | V-1135 |
| 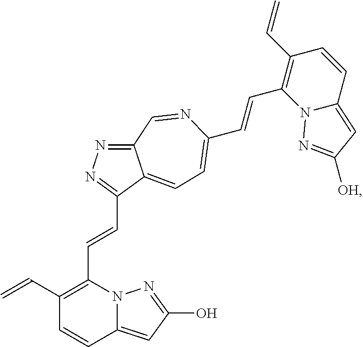 [SMILES: C=Cc1ccc2cc(O)nn2c1/C=C/c1ccc2c(/C=C/c3c(C=C)ccc4cc(O)nn34)nnc-2cn1] | V-1136 |
| 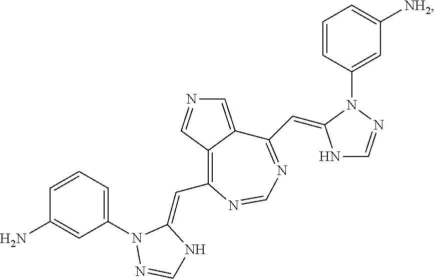 [SMILES: Nc1cccc(N2N=CN/C2=C\c2ncnc(/C=C3\NC=NN3c3cccc(N)c3)c3cncc2-3)c1] | V-1137 |
| 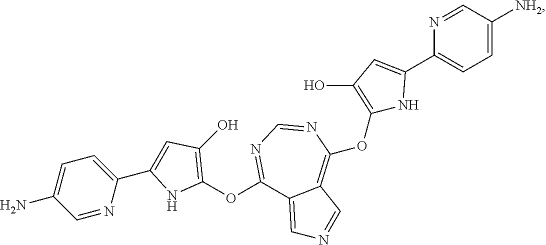 [SMILES: Nc1ccc(-c2cc(O)c(Oc3ncnc(Oc4[nH]c(-c5ccc(N)cn5)cc4O)c4cncc3-4)[nH]2)nc1] | V-1138 |
| 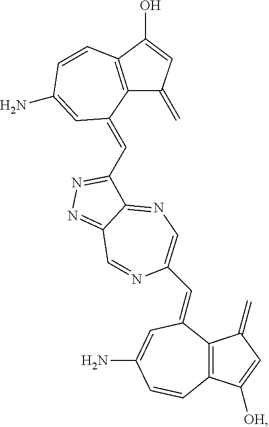 [SMILES: C=c1cc(O)c2ccc(N)c/c(=C\c3cnc4c(/C=c5\cc(N)ccc6c(O)cc(=C)c5=6)nnc-4cn3)c1=2] | V-1139 |
| 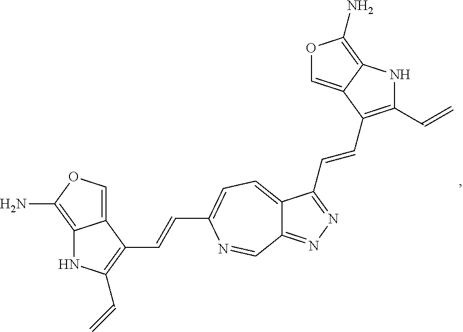 [SMILES: C=Cc1[nH]c2c(N)occ2c1/C=C/c1ccc2c(/C=C/c3c(C=C)[nH]c4c(N)occ34)nnc-2cn1] | V-1140 |
| 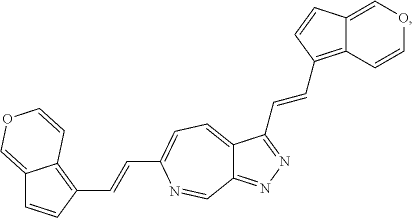 [SMILES: C(=C\c1ccc2coccc1-2)/c1ccc2c(/C=C/c3ccc4coccc3-4)nnc-2cn1] | V-1141 |
| 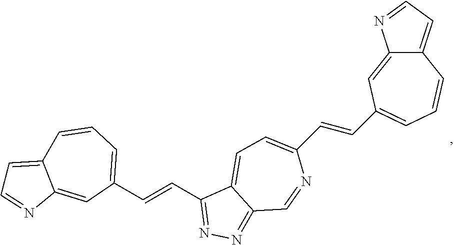 [SMILES: C(=C\c1ccc2c(/C=C/c3cccc4ccnc-4c3)nnc-2cn1)/c1cccc2ccnc-2c1] | V-1142 |
| 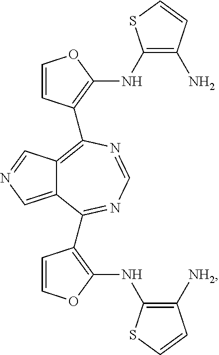 [SMILES: Nc1ccsc1Nc1occc1-c1ncnc(-c2ccoc2Nc2sccc2N)c2cncc1-2] | V-1143 |
| 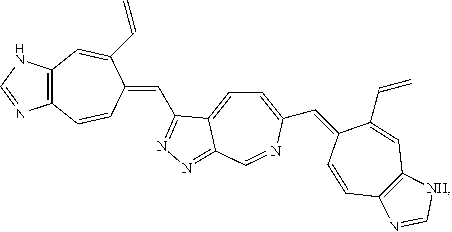 [SMILES: C=CC1=Cc2[nH]cnc2C=C/C1=C\c1ccc2c(/C=C3\C=Cc4nc[nH]c4C=C3C=C)nnc-2cn1] | V-1144 |
| 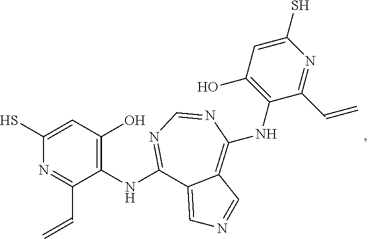 [SMILES: C=Cc1nc(S)cc(O)c1Nc1ncnc(Nc2c(O)cc(S)nc2C=C)c2cncc1-2] | V-1145 |
| 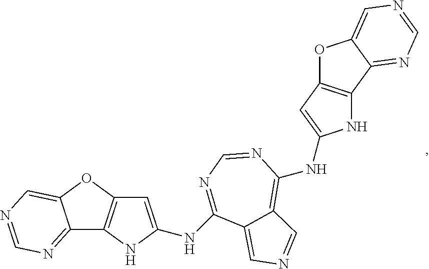 [SMILES: c1nc(Nc2cc3oc4cncnc4c3[nH]2)c2cncc-2c(Nc2cc3oc4cncnc4c3[nH]2)n1] | V-1146 |
| 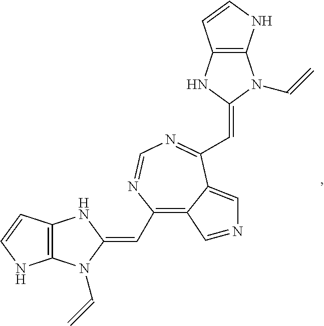 [SMILES: C=CN1/C(=C/c2ncnc(/C=C3\Nc4cc[nH]c4N3C=C)c3cncc2-3)Nc2cc[nH]c21] | V-1147 |
| 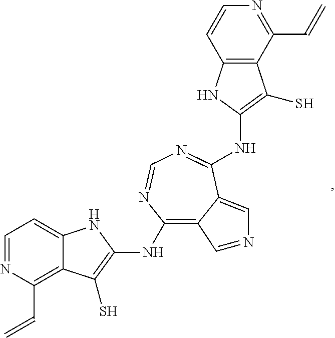 [SMILES: C=Cc1nccc2[nH]c(Nc3ncnc(Nc4[nH]c5ccnc(C=C)c5c4S)c4cncc3-4)c(S)c12] | V-1148 |
| 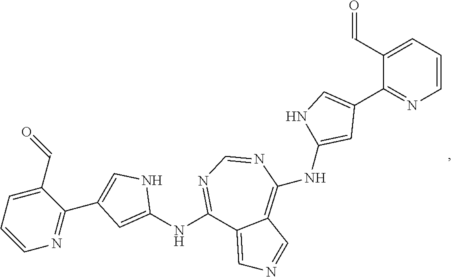 [SMILES: O=Cc1cccnc1-c1c[nH]c(Nc2ncnc(Nc3cc(-c4ncccc4C=O)c[nH]3)c3cncc2-3)c1] | V-1149 |
| 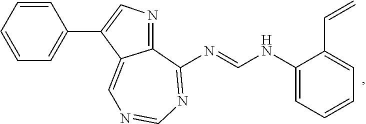 [SMILES: C=Cc1ccccc1N/C=N/c1ncncc2c(-c3ccccc3)cnc1-2] | V-1150 |
| 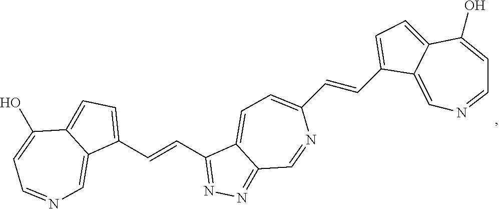 [SMILES: Oc1ccncc2c(/C=C/c3ccc4c(/C=C/c5ccc6c(O)ccncc5-6)nnc-4cn3)ccc1-2] | V-1151 |
| 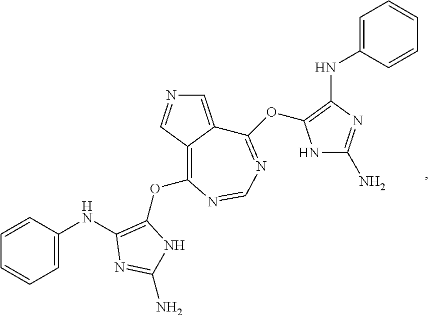 [SMILES: Nc1nc(Nc2ccccc2)c(Oc2ncnc(Oc3[nH]c(N)nc3Nc3ccccc3)c3cncc2-3)[nH]1] | V-1152 |
| 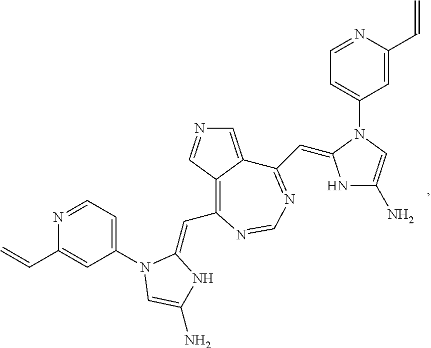 [SMILES: C=Cc1cc(N2C=C(N)N/C2=C\c2ncnc(/C=C3\NC(N)=CN3c3ccnc(C=C)c3)c3cncc2-3)ccn1] | V-1153 |
| 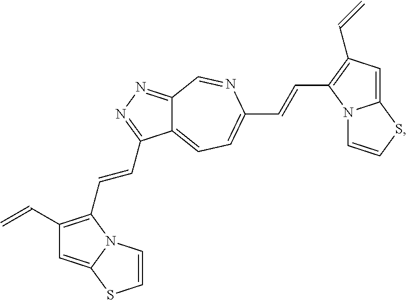 [SMILES: C=Cc1cc2sccn2c1/C=C/c1ccc2c(/C=C/c3c(C=C)cc4sccn34)nnc-2cn1] | V-1154 |
| 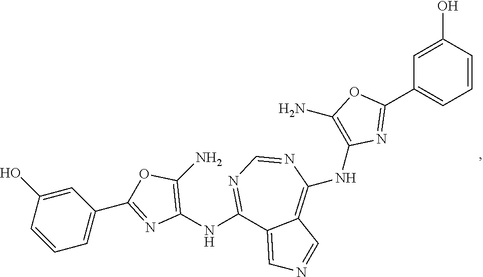 [SMILES: Nc1oc(-c2cccc(O)c2)nc1Nc1ncnc(Nc2nc(-c3cccc(O)c3)oc2N)c2cncc1-2] | V-1155 |
| 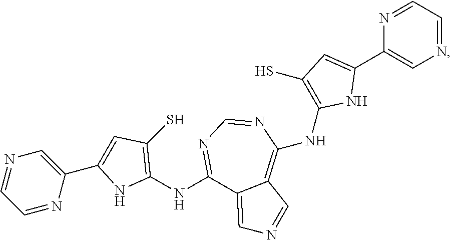 [SMILES: Sc1cc(-c2cnccn2)[nH]c1Nc1ncnc(Nc2[nH]c(-c3cnccn3)cc2S)c2cncc1-2] | V-1156 |
| 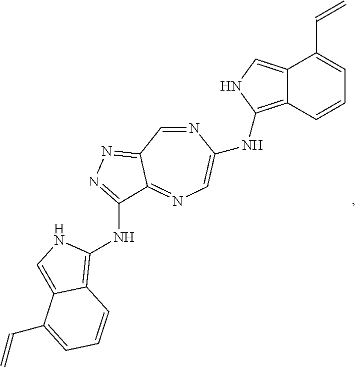 [SMILES: C=Cc1cccc2c(Nc3cnc4c(Nc5[nH]cc6c(C=C)cccc56)nnc-4cn3)[nH]cc12] | V-1157 |
| 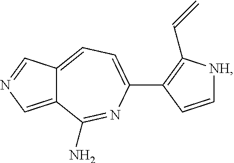 [SMILES: C=Cc1[nH]ccc1-c1ccc2cncc-2c(N)n1] | V-1158 |
| 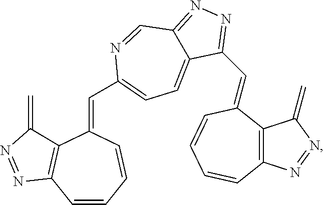 [SMILES: C=c1nnc2cccc/c(=C\c3ccc4c(/C=c5\ccccc6nnc(=C)c5=6)nnc-4cn3)c1=2] | V-1159 |
| 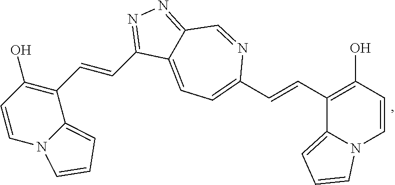 [SMILES: Oc1ccn2cccc2c1/C=C/c1ccc2c(/C=C/c3c(O)ccn4cccc34)nnc-2cn1] | V-1160 |
| 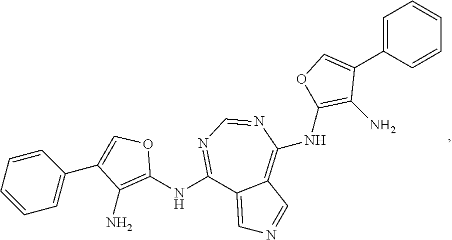 [SMILES: Nc1c(-c2ccccc2)coc1Nc1ncnc(Nc2occ(-c3ccccc3)c2N)c2cncc1-2] | V-1161 |
| 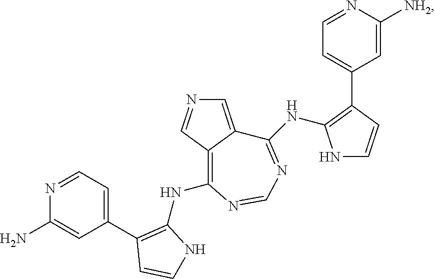 [SMILES: Nc1cc(-c2cc[nH]c2Nc2ncnc(Nc3[nH]ccc3-c3ccnc(N)c3)c3cncc2-3)ccn1] | V-1162 |
| 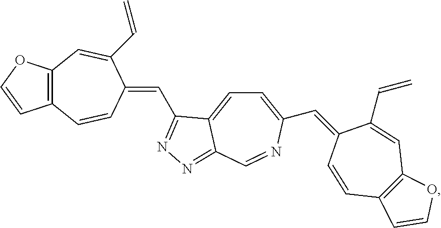 [SMILES: C=CC1=Cc2occc2C=C/C1=C\c1ccc2c(/C=C3\C=Cc4ccoc4C=C3C=C)nnc-2cn1] | V-1163 |
| 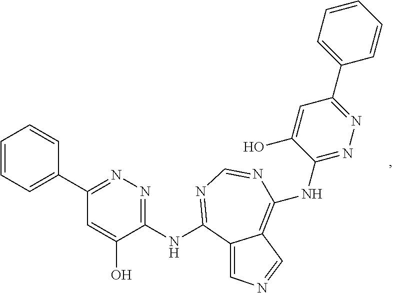 [SMILES: Oc1cc(-c2ccccc2)nnc1Nc1ncnc(Nc2nnc(-c3ccccc3)cc2O)c2cncc1-2] | V-1164 |
| 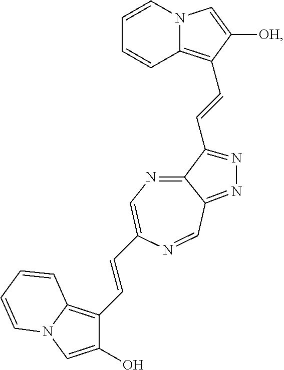 [SMILES: Oc1cn2ccccc2c1/C=C/c1cnc2c(/C=C/c3c(O)cn4ccccc34)nnc-2cn1] | V-1165 |
| 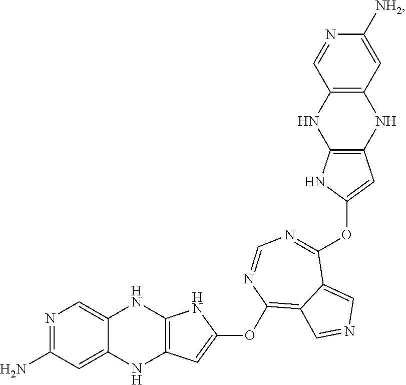 [SMILES: Nc1cc2c(cn1)Nc1[nH]c(Oc3ncnc(Oc4cc5c([nH]4)Nc4cnc(N)cc4N5)c4cncc3-4)cc1N2] | V-1166 |
| 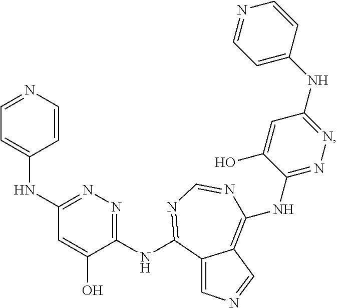 [SMILES: Oc1cc(Nc2ccncc2)nnc1Nc1ncnc(Nc2nnc(Nc3ccncc3)cc2O)c2cncc1-2] | V-1167 |
| 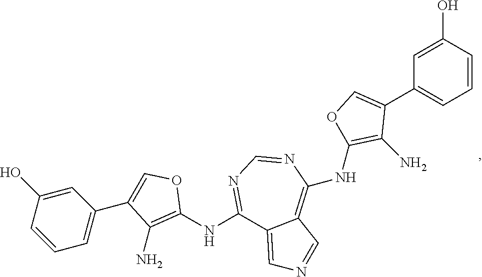 [SMILES: Nc1c(-c2cccc(O)c2)coc1Nc1ncnc(Nc2occ(-c3cccc(O)c3)c2N)c2cncc1-2] | V-1168 |
| 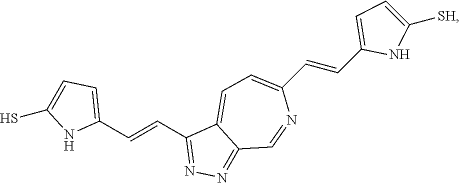 [SMILES: Sc1ccc(/C=C/c2ccc3c(/C=C/c4ccc(S)[nH]4)nnc-3cn2)[nH]1] | V-1169 |
| 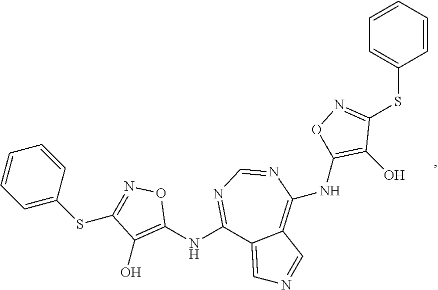 [SMILES: Oc1c(Sc2ccccc2)noc1Nc1ncnc(Nc2onc(Sc3ccccc3)c2O)c2cncc1-2] | V-1170 |
| 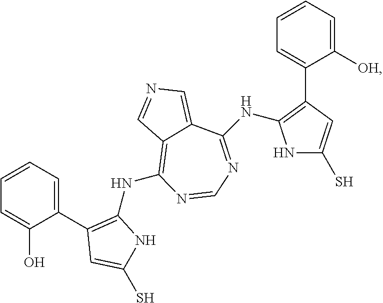 [SMILES: Oc1ccccc1-c1cc(S)[nH]c1Nc1ncnc(Nc2[nH]c(S)cc2-c2ccccc2O)c2cncc1-2] | V-1171 |
| 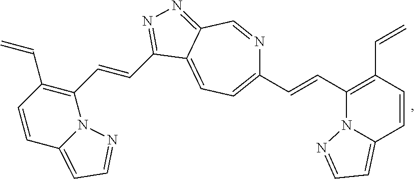 [SMILES: C=Cc1ccc2ccnn2c1/C=C/c1ccc2c(/C=C/c3c(C=C)ccc4ccnn34)nnc-2cn1] | V-1172 |
| 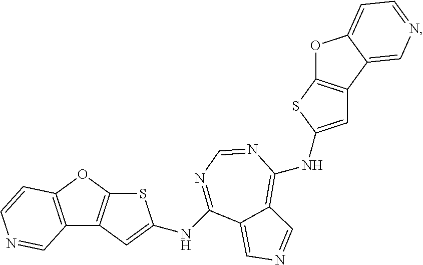 [SMILES: c1cc2oc3sc(Nc4ncnc(Nc5cc6c(oc7ccncc76)s5)c5cncc4-5)cc3c2cn1] | V-1173 |
| 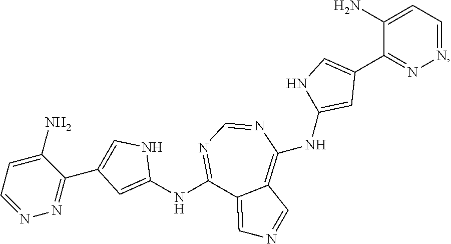 [SMILES: Nc1ccnnc1-c1c[nH]c(Nc2ncnc(Nc3cc(-c4nnccc4N)c[nH]3)c3cncc2-3)c1] | V-1174 |
| 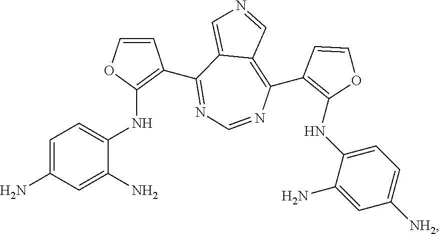 [SMILES: Nc1ccc(Nc2occc2-c2ncnc(-c3ccoc3Nc3ccc(N)cc3N)c3cncc2-3)c(N)c1] | V-1175 |
| 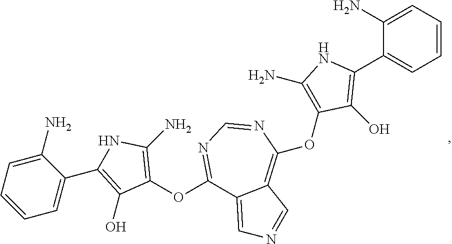 [SMILES: Nc1ccccc1-c1[nH]c(N)c(Oc2ncnc(Oc3c(N)[nH]c(-c4ccccc4N)c3O)c3cncc2-3)c1O] | V-1176 |
| 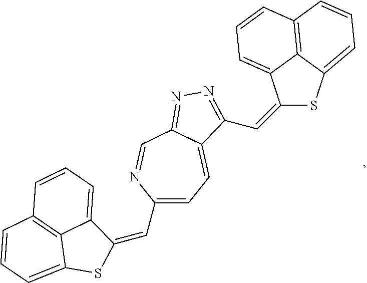 [SMILES: C(/c1ccc2c(/C=c3/sc4cccc5cccc3c54)nnc-2cn1)=c1\sc2cccc3cccc1c32] | V-1177 |
| 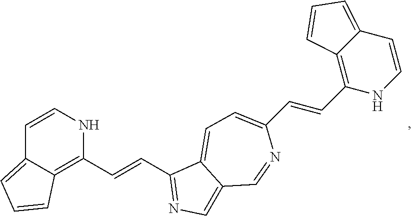 [SMILES: C(=C\c1[nH]ccc2cccc1-2)/c1ccc2c(/C=C/c3[nH]ccc4cccc3-4)ncc-2cn1] | V-1178 |
| 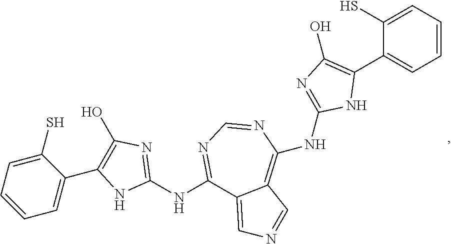 [SMILES: Oc1nc(Nc2ncnc(Nc3nc(O)c(-c4ccccc4S)[nH]3)c3cncc2-3)[nH]c1-c1ccccc1S] | V-1179 |
| 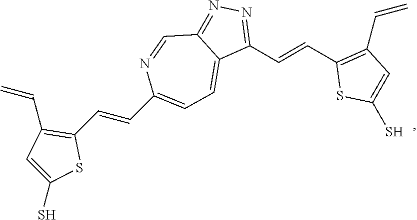 [SMILES: C=Cc1cc(S)sc1/C=C/c1ccc2c(/C=C/c3sc(S)cc3C=C)nnc-2cn1] | V-1180 |
| 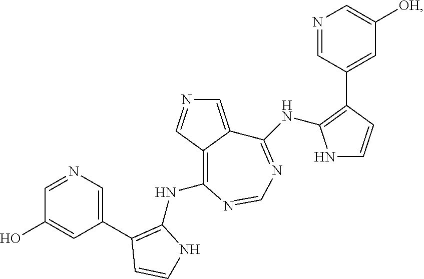 [SMILES: Oc1cncc(-c2cc[nH]c2Nc2ncnc(Nc3[nH]ccc3-c3cncc(O)c3)c3cncc2-3)c1] | V-1181 |
| 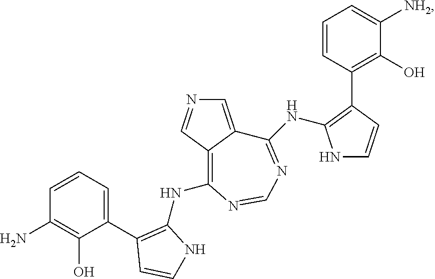 [SMILES: Nc1cccc(-c2cc[nH]c2Nc2ncnc(Nc3[nH]ccc3-c3cccc(N)c3O)c3cncc2-3)c1O] | V-1182 |
| 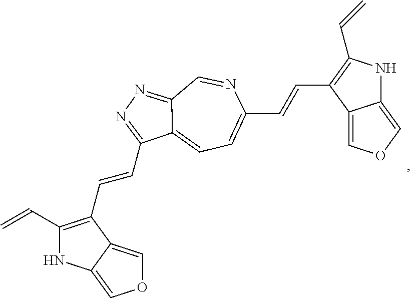 [SMILES: C=Cc1[nH]c2cocc2c1/C=C/c1ccc2c(/C=C/c3c(C=C)[nH]c4cocc34)nnc-2cn1] | V-1183 |
| 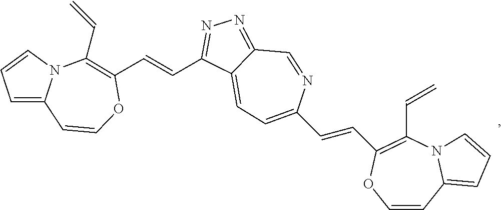 [SMILES: C=CC1=C(/C=C/c2ccc3c(/C=C/C4=C(C=C)n5cccc5C=CO4)nnc-3cn2)OC=Cc2cccn21] | V-1184 |
| 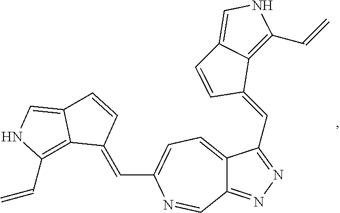 [SMILES: C=Cc1[nH]cc2c1/C(=C/c1ccc3c(/C=C4\C=Cc5c[nH]c(C=C)c54)nnc-3cn1)C=C2] | V-1185 |
| 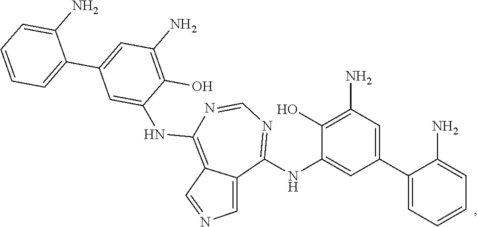 [SMILES: Nc1ccccc1-c1cc(N)c(O)c(Nc2ncnc(Nc3cc(-c4ccccc4N)cc(N)c3O)c3cncc2-3)c1] | V-1186 |
| 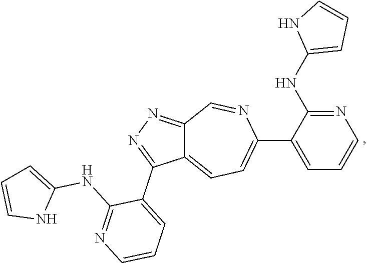 [SMILES: c1c[nH]c(Nc2ncccc2-c2ccc3c(-c4cccnc4Nc4ccc[nH]4)nnc-3cn2)c1] | V-1187 |
| 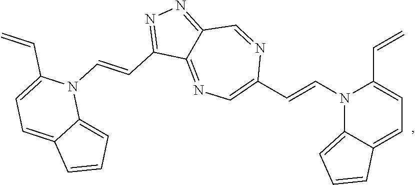 [SMILES: C=Cc1ccc2cccc-2n1/C=C/c1cnc2c(/C=C/n3c(C=C)ccc4cccc3-4)nnc-2cn1] | V-1188 |
| 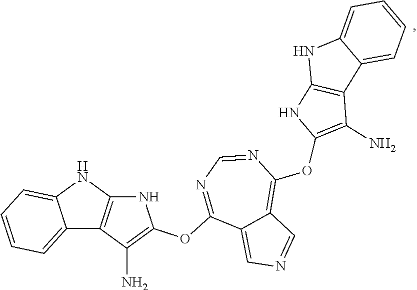 [SMILES: Nc1c(Oc2ncnc(Oc3[nH]c4[nH]c5ccccc5c4c3N)c3cncc2-3)[nH]c2[nH]c3ccccc3c12] | V-1189 |
| 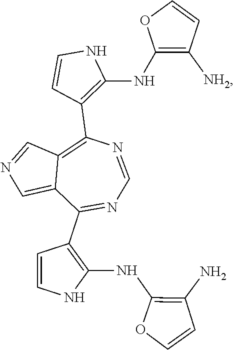 [SMILES: Nc1ccoc1Nc1[nH]ccc1-c1ncnc(-c2cc[nH]c2Nc2occc2N)c2cncc1-2] | V-1190 |
| 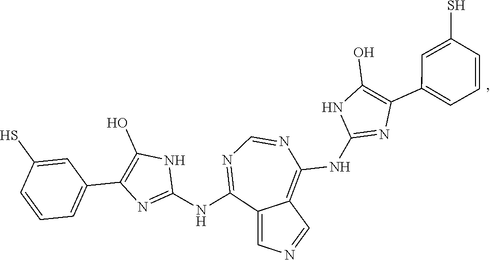 [SMILES: Oc1[nH]c(Nc2ncnc(Nc3nc(-c4cccc(S)c4)c(O)[nH]3)c3cncc2-3)nc1-c1cccc(S)c1] | V-1191 |
| 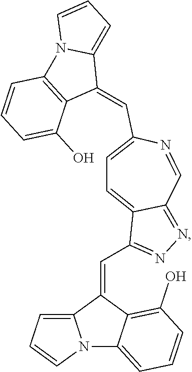 [SMILES: Oc1cccc2c1/c(=C\c1ccc3c(/C=c4\c5c(O)cccc5n5cccc45)nnc-3cn1)c1cccn12] | V-1192 |
| 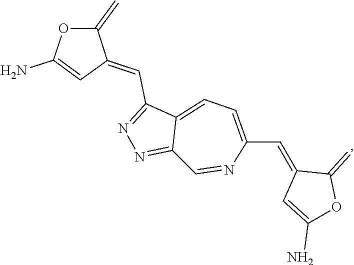 | V-1193 |
| 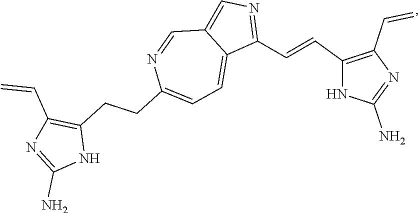 | V-1194 |
| 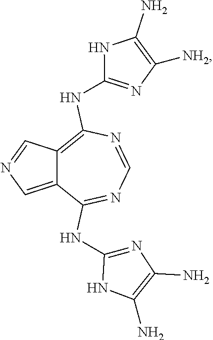 [SMILES: Nc1nc(Nc2ncnc(Nc3nc(N)c(N)[nH]3)c3cncc2-3)[nH]c1N] | V-1195 |
| 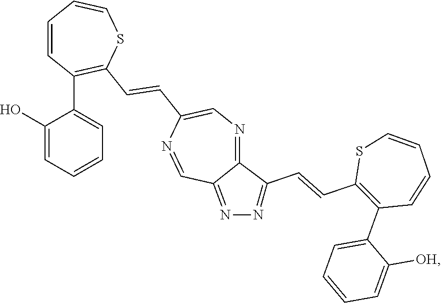 [SMILES: Oc1ccccc1C1=C(/C=C/c2cnc3c(/C=C/C4=C(c5ccccc5O)C=CC=CS4)nnc-3cn2)SC=CC=C1] | V-1196 |
| 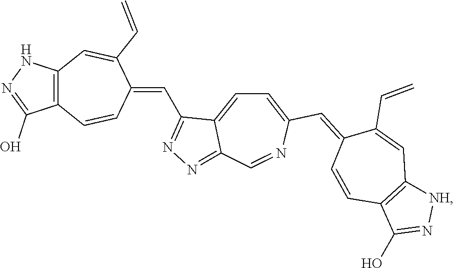 [SMILES: C=CC1=Cc2[nH]nc(O)c2C=C/C1=C\c1ccc2c(/C=C3\C=Cc4c(O)n[nH]c4C=C3C=C)nnc-2cn1] | V-1197 |
| 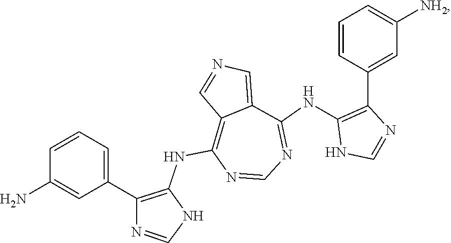 [SMILES: Nc1cccc(-c2nc[nH]c2Nc2ncnc(Nc3[nH]cnc3-c3cccc(N)c3)c3cncc2-3)c1] | V-1198 |
| 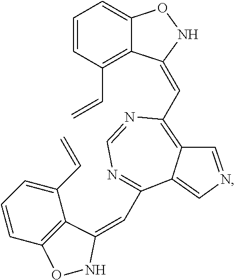 [SMILES: C=Cc1cccc2c1/C(=C\c1ncnc(/C=C3/NOc4cccc(C=C)c43)c3cncc1-3)NO2] | V-1199 |
| 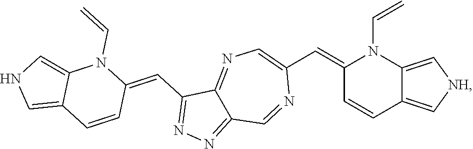 [SMILES: C=CN1/C(=C/c2cnc3c(/C=C4\C=Cc5c[nH]cc5N4C=C)nnc-3cn2)C=Cc2c[nH]cc21] | V-1200 |
| 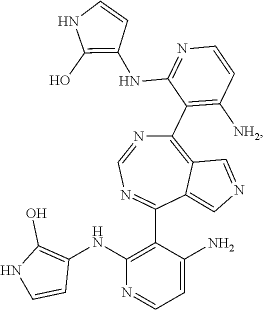 [SMILES: Nc1ccnc(Nc2cc[nH]c2O)c1-c1ncnc(-c2c(N)ccnc2Nc2cc[nH]c2O)c2cncc1-2] | V-1201 |
| 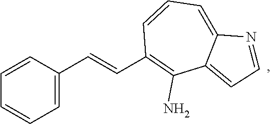 [SMILES: Nc1c(/C=C/c2ccccc2)cccc2nccc1-2] | V-1202 |
| 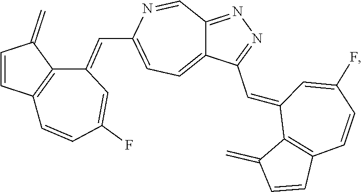 [SMILES: C=c1ccc2ccc(F)c/c(=C\c3ccc4c(/C=c5\cc(F)ccc6ccc(=C)c5=6)nnc-4cn3)c1=2] | V-1203 |
| 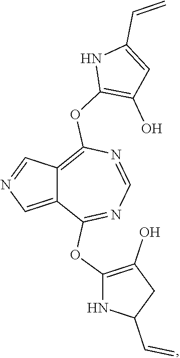 | V-1204 |
| 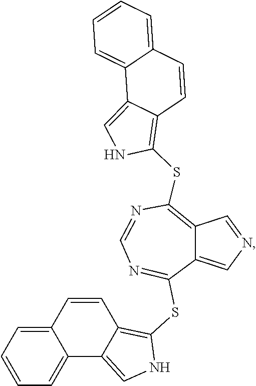 [SMILES: c1ccc2c(c1)ccc1c(Sc3ncnc(Sc4[nH]cc5c4ccc4ccccc45)c4cncc3-4)[nH]cc12] | V-1205 |
| 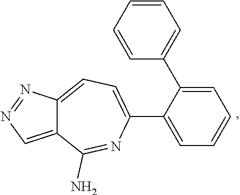 [SMILES: Nc1nc(-c2ccccc2-c2ccccc2)ccc2nncc1-2] | V-1206 |
| 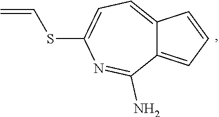 | V-1207 |
| 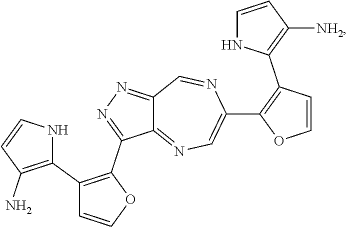 [SMILES: Nc1cc[nH]c1-c1ccoc1-c1cnc2c(-c3occc3-c3[nH]ccc3N)nnc-2cn1] | V-1208 |
| 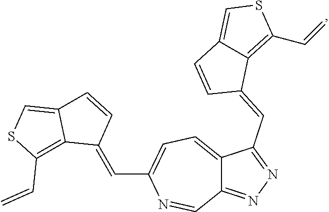 | V-1209 |
| 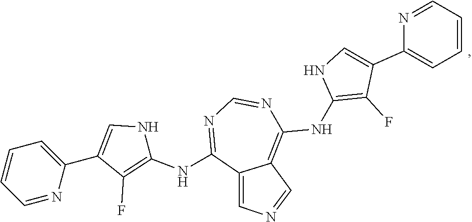 [SMILES: Fc1c(-c2ccccn2)c[nH]c1Nc1ncnc(Nc2[nH]cc(-c3ccccn3)c2F)c2cncc1-2] | V-1210 |
| 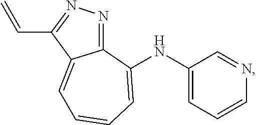 [SMILES: C=Cc1nnc2c(Nc3cccnc3)ccccc1-2] | V-1211 |
| 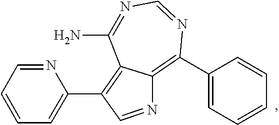 [SMILES: Nc1ncnc(-c2ccccc2)c2ncc(-c3ccccn3)c1-2] | V-1212 |
| 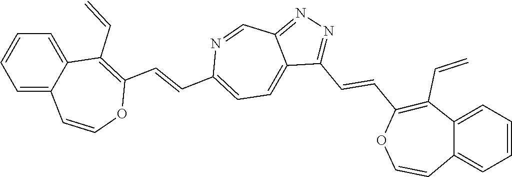 [SMILES: C=CC1=C(/C=C/c2ccc3c(/C=C/C4=C(C=C)c5ccccc5C=CO4)nnc-3cn2)OC=Cc2ccccc21] | V-1213 |
| 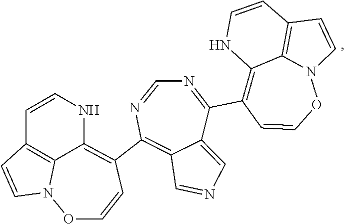 [SMILES: c1nc(-c2ccon3ccc4cc[nH]c2-c43)c2cncc-2c(-c2ccon3ccc4cc[nH]c2-c43)n1] | V-1214 |
| 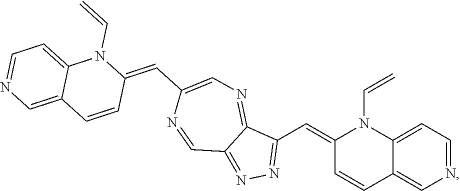 [SMILES: C=CN1/C(=C/c2cnc3c(/C=C4\C=Cc5cnccc5N4C=C)nnc-3cn2)C=Cc2cnccc21] | V-1215 |
| 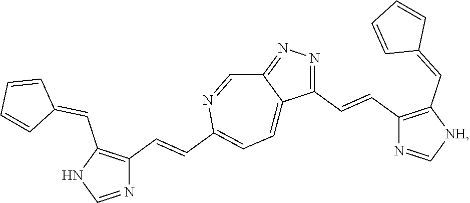 [SMILES: C1=CC(=Cc2[nH]cnc2/C=C/c2ccc3c(/C=C/c4nc[nH]c4C=C4C=CC=C4)nnc-3cn2)C=C1] | V-1216 |
| 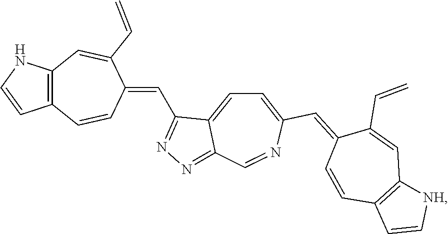 [SMILES: C=CC1=Cc2[nH]ccc2C=C/C1=C\c1ccc2c(/C=C3\C=Cc4cc[nH]c4C=C3C=C)nnc-2cn1] | V-1217 |
| 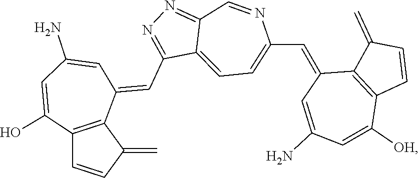 [SMILES: C=c1ccc2c(O)cc(N)c/c(=C\c3ccc4c(/C=c5\cc(N)cc(O)c6ccc(=C)c5=6)nnc-4cn3)c1=2] | V-1218 |
| 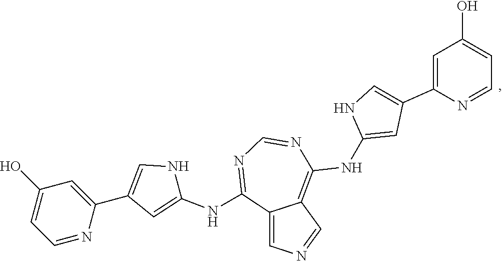 [SMILES: Oc1ccnc(-c2c[nH]c(Nc3ncnc(Nc4cc(-c5cc(O)ccn5)c[nH]4)c4cncc3-4)c2)c1] | V-1219 |
| 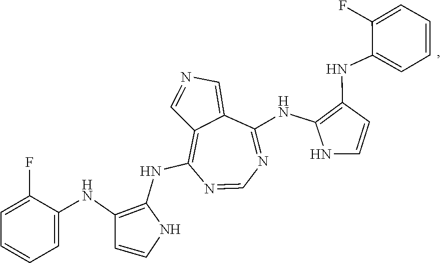 [SMILES: Fc1ccccc1Nc1cc[nH]c1Nc1ncnc(Nc2[nH]ccc2Nc2ccccc2F)c2cncc1-2] | V-1220 |
| 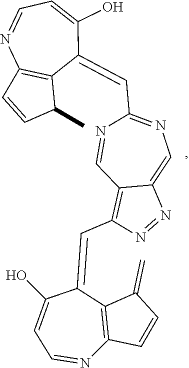 [SMILES: C=c1ccc2nccc(O)/c(=C/c3nnc4cnc(/C=C5/C(O)=CC=NC6=C5[C@@H](C)C=C6)ncc3-4)c1=2] | V-1221 |
| 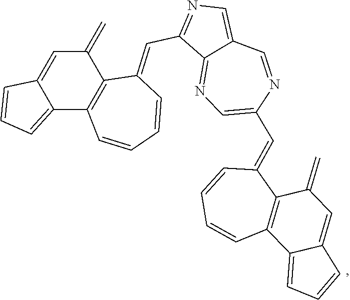 [SMILES: C=c1cc2cccc2c2cccc/c(=C\c3cnc4c(/C=c5\ccccc6c5c(=C)cc5cccc56)ncc-4cn3)c12] | V-1222 |
| 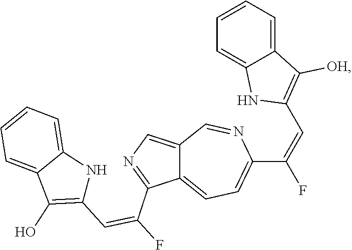 [SMILES: Oc1c(/C=C(/F)c2ccc3c(/C(F)=C\c4[nH]c5ccccc5c4O)ncc-3cn2)[nH]c2ccccc12] | V-1223 |
| 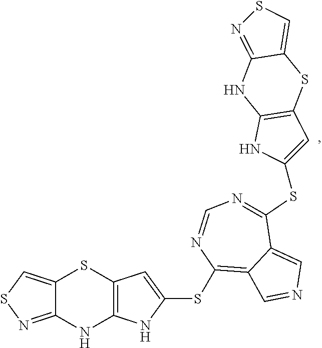 [SMILES: c1nc(Sc2cc3c([nH]2)Nc2nscc2S3)c2cncc-2c(Sc2cc3c([nH]2)Nc2nscc2S3)n1] | V-1224 |
| 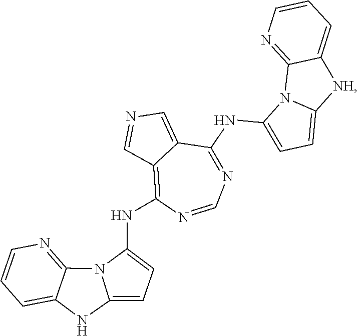 [SMILES: c1cnc2c(c1)[nH]c1ccc(Nc3ncnc(Nc4ccc5[nH]c6cccnc6n45)c4cncc3-4)n12] | V-1225 |
| 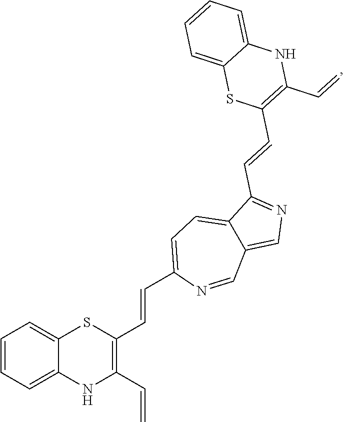 | V-1226 |
| 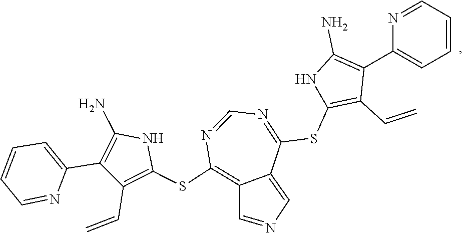 [SMILES: C=Cc1c(Sc2ncnc(Sc3[nH]c(N)c(-c4ccccn4)c3C=C)c3cncc2-3)[nH]c(N)c1-c1ccccn1] | V-1227 |
| 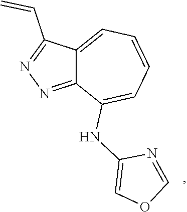 [SMILES: C=Cc1nnc2c(Nc3cocn3)ccccc1-2] | V-1228 |
| 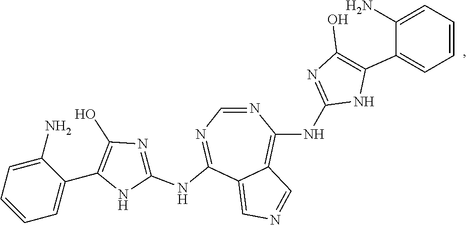 [SMILES: Nc1ccccc1-c1[nH]c(Nc2ncnc(Nc3nc(O)c(-c4ccccc4N)[nH]3)c3cncc2-3)nc1O] | V-1229 |
| 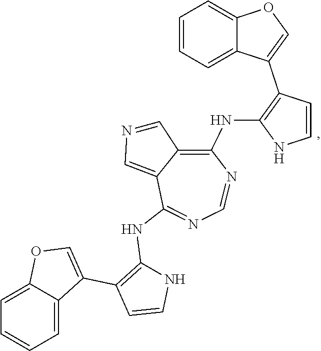 [SMILES: c1ccc2c(-c3cc[nH]c3Nc3ncnc(Nc4[nH]ccc4-c4coc5ccccc45)c4cncc3-4)coc2c1] | V-1230 |
| 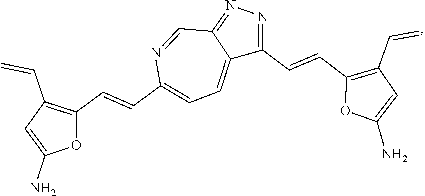 | V-1231 |
| 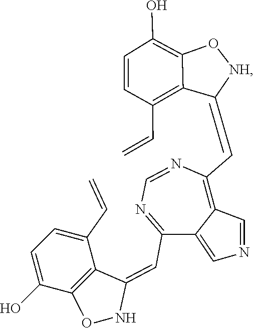 [SMILES: C=Cc1ccc(O)c2c1/C(=C\c1ncnc(/C=C3/NOc4c(O)ccc(C=C)c43)c3cncc1-3)NO2] | V-1232 |
| 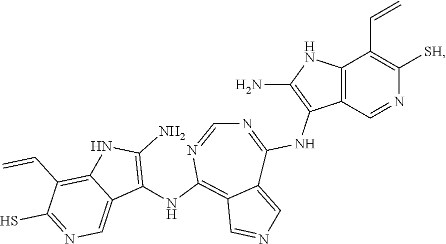 [SMILES: C=Cc1c(S)ncc2c(Nc3ncnc(Nc4c(N)[nH]c5c(C=C)c(S)ncc45)c4cncc3-4)c(N)[nH]c12] | V-1233 |
| 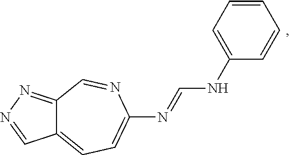 [SMILES: C(=N\c1ccc2cnnc-2cn1)/Nc1ccccc1] | V-1234 |
| 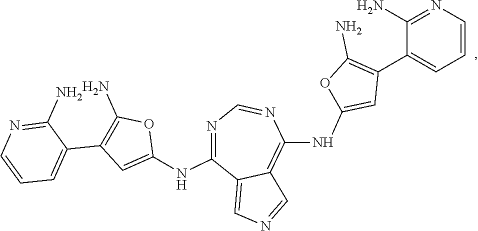 [SMILES: Nc1ncccc1-c1cc(Nc2ncnc(Nc3cc(-c4cccnc4N)c(N)o3)c3cncc2-3)oc1N] | V-1235 |
| 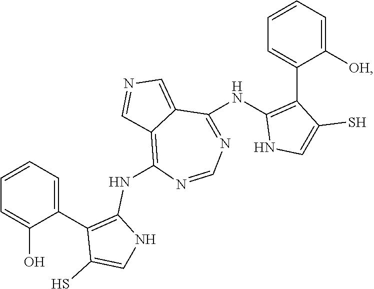 [SMILES: Oc1ccccc1-c1c(S)c[nH]c1Nc1ncnc(Nc2[nH]cc(S)c2-c2ccccc2O)c2cncc1-2] | V-1236 |
| 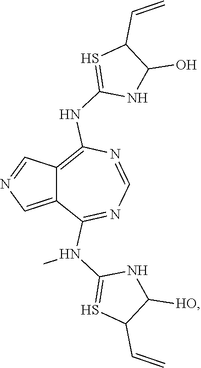 | V-1237 |
| 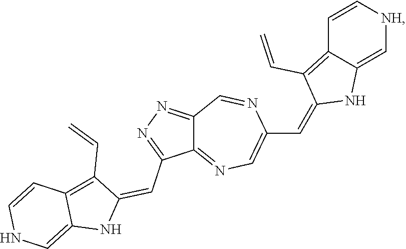 [SMILES: C=Cc1c2c([nH]/c1=C/c1cnc3c(/C=c4/[nH]c5c(c4C=C)C=CNC=5)nnc-3cn1)=CNC=C2] | V-1238 |
| 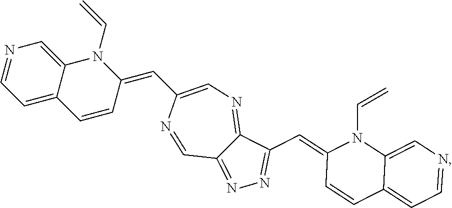 [SMILES: C=CN1/C(=C/c2cnc3c(/C=C4\C=Cc5ccncc5N4C=C)nnc-3cn2)C=Cc2ccncc21] | V-1239 |
| 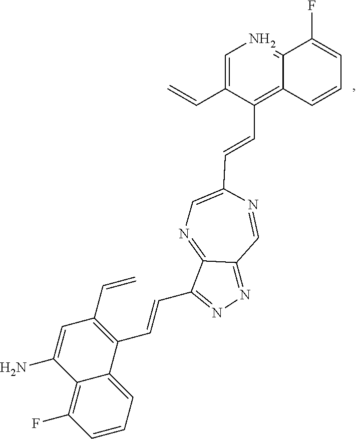 [SMILES: C=Cc1cnc2c(F)cccc2c1/C=C/c1cnc2c(/C=C/c3c(C=C)cc(N)c4c(F)cccc34)nnc-2cn1] | V-1240 |
| 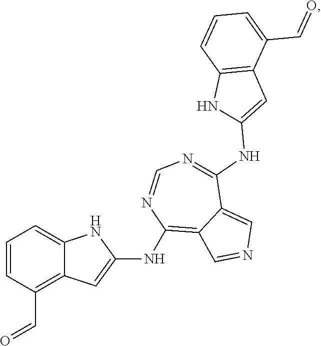 [SMILES: O=Cc1cccc2[nH]c(Nc3ncnc(Nc4cc5c(C=O)cccc5[nH]4)c4cncc3-4)cc12] | V-1241 |
| 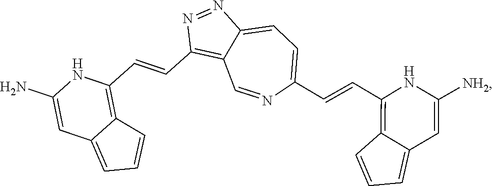 [SMILES: Nc1cc2cccc-2c(/C=C/c2ccc3nnc(/C=C/c4[nH]c(N)cc5cccc4-5)c-3cn2)[nH]1] | V-1242 |
| 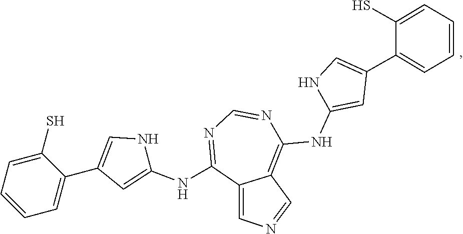 [SMILES: Sc1ccccc1-c1c[nH]c(Nc2ncnc(Nc3cc(-c4ccccc4S)c[nH]3)c3cncc2-3)c1] | V-1243 |
| 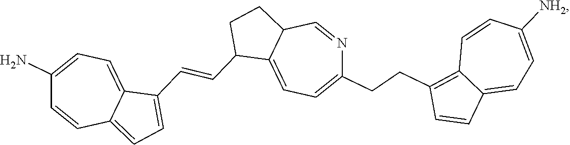 [SMILES: Nc1ccc2ccc(/C=C/C3CCC4C=NC(CCc5ccc6ccc(N)ccc5-6)=CC=C43)c-2cc1] | V-1244 |
| 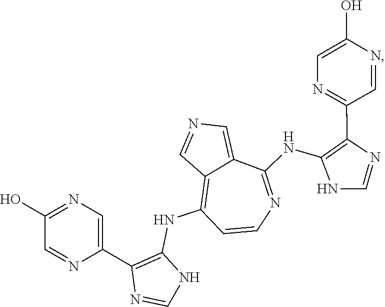 [SMILES: Oc1cnc(-c2nc[nH]c2Nc2ccnc(Nc3[nH]cnc3-c3cnc(O)cn3)c3cncc2-3)cn1] | V-1245 |
| 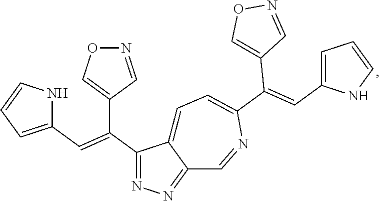 [SMILES: C(=C(/c1cnoc1)c1ccc2c(/C(=C/c3ccc[nH]3)c3cnoc3)nnc-2cn1)/c1ccc[nH]1] | V-1246 |
| 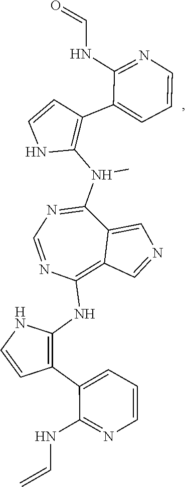 [SMILES: C=CNc1ncccc1-c1cc[nH]c1Nc1ncnc(N(C)c2[nH]ccc2-c2cccnc2NC=O)c2cncc1-2] | V-1247 |
| 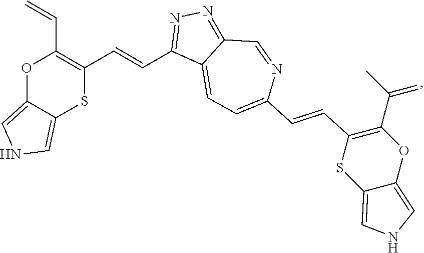 | V-1248 |
| 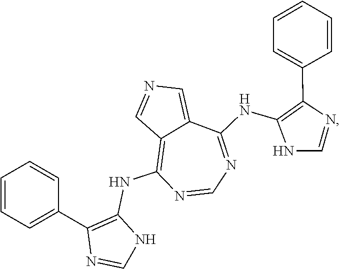 [SMILES: c1ccc(-c2nc[nH]c2Nc2ncnc(Nc3[nH]cnc3-c3ccccc3)c3cncc2-3)cc1] | V-1249 |
| 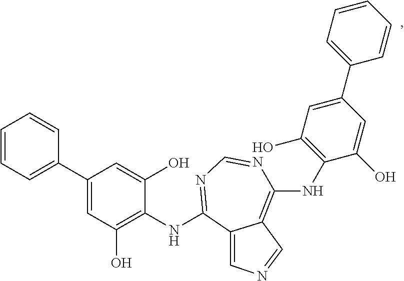 [SMILES: Oc1cc(-c2ccccc2)cc(O)c1Nc1ncnc(Nc2c(O)cc(-c3ccccc3)cc2O)c2cncc1-2] | V-1250 |
| 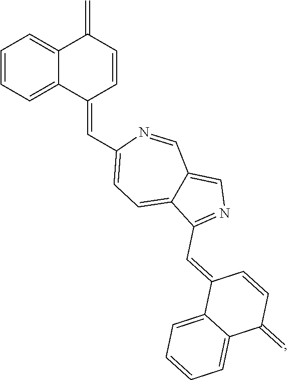 | V-1251 |
| 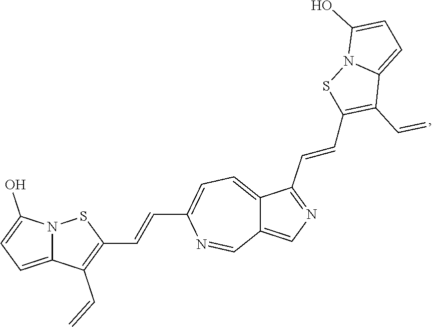 | V-1252 |
| 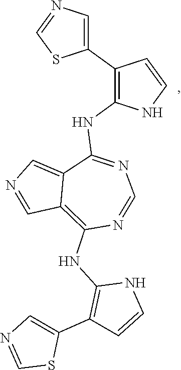 [SMILES: c1nc(Nc2[nH]ccc2-c2cncs2)c2cncc-2c(Nc2[nH]ccc2-c2cncs2)n1] | V-1253 |
| 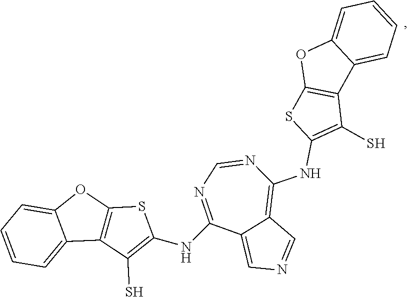 [SMILES: Sc1c(Nc2ncnc(Nc3sc4oc5ccccc5c4c3S)c3cncc2-3)sc2oc3ccccc3c12] | V-1254 |
| 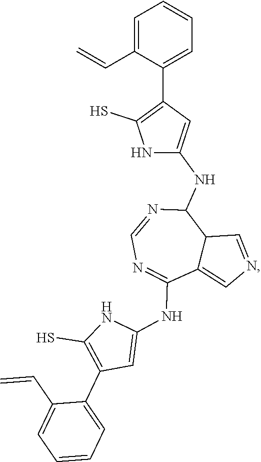 [SMILES: C=Cc1ccccc1-c1cc(NC2=NC=NC(Nc3cc(-c4ccccc4C=C)c(S)[nH]3)C3C=NC=C23)[nH]c1S] | V-1255 |
| 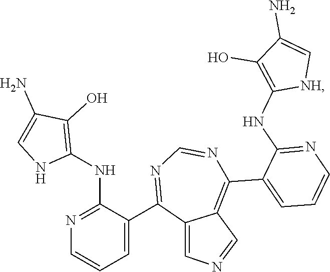 [SMILES: Nc1c[nH]c(Nc2ncccc2-c2ncnc(-c3cccnc3Nc3[nH]cc(N)c3O)c3cncc2-3)c1O] | V-1256 |
| 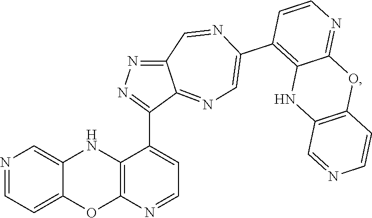 [SMILES: c1cc2c(cn1)Nc1c(-c3cnc4c(-c5ccnc6c5Nc5cnccc5O6)nnc-4cn3)ccnc1O2] | V-1257 |
| 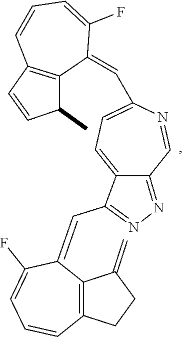 [SMILES: C=C1CCC2=C1/C(=C\c1nnc3cnc(/C=C4/C(F)=CC=CC5=C4[C@@H](C)C=C5)ccc1-3)C(F)=CC=C2] | V-1258 |
| 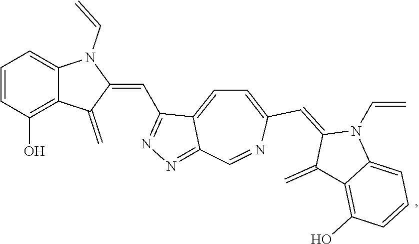 [SMILES: C=Cn1/c(=C/c2ccc3c(/C=c4\c(=C)c5c(O)cccc5n4C=C)nnc-3cn2)c(=C)c2c(O)cccc21] | V-1259 |
| 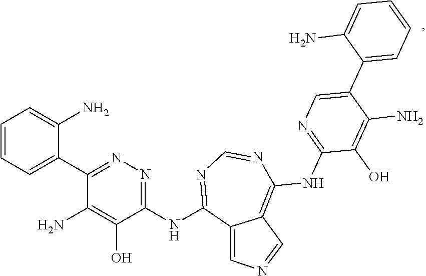 [SMILES: Nc1ccccc1-c1cnc(Nc2ncnc(Nc3nnc(-c4ccccc4N)c(N)c3O)c3cncc2-3)c(O)c1N] | V-1260 |
| 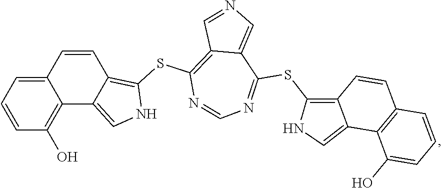 [SMILES: Oc1cccc2ccc3c(Sc4ncnc(Sc5[nH]cc6c5ccc5cccc(O)c56)c5cncc4-5)[nH]cc3c12] | V-1261 |
| 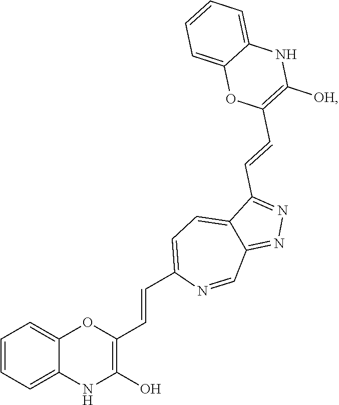 [SMILES: OC1=C(/C=C/c2ccc3c(/C=C/C4=C(O)Nc5ccccc5O4)nnc-3cn2)Oc2ccccc2N1] | V-1262 |
| 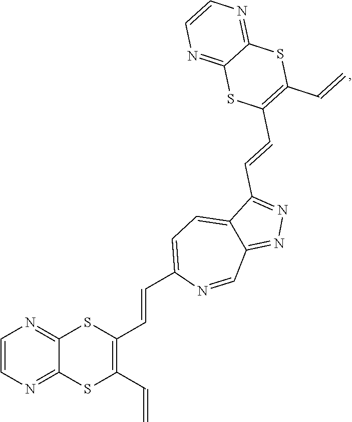 [SMILES: C=CC1=C(/C=C/c2ccc3c(/C=C/C4=C(C=C)Sc5nccnc5S4)nnc-3cn2)Sc2nccnc2S1] | V-1263 |
| 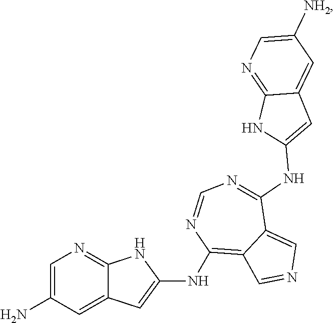 [SMILES: Nc1cnc2[nH]c(Nc3ncnc(Nc4cc5cc(N)cnc5[nH]4)c4cncc3-4)cc2c1] | V-1264 |
| 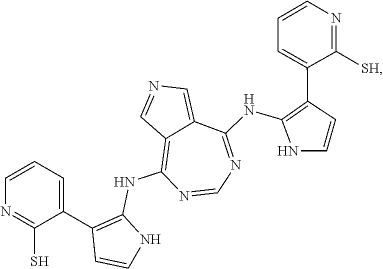 [SMILES: Sc1ncccc1-c1cc[nH]c1Nc1ncnc(Nc2[nH]ccc2-c2cccnc2S)c2cncc1-2] | V-1265 |
| 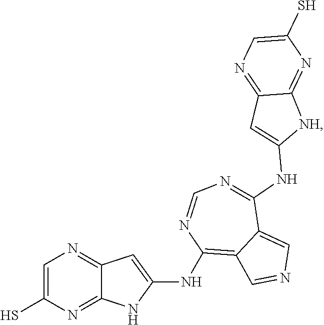 [SMILES: Sc1cnc2cc(Nc3ncnc(Nc4cc5ncc(S)nc5[nH]4)c4cncc3-4)[nH]c2n1] | V-1266 |
| 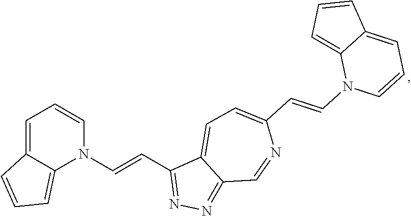 [SMILES: C(=C\n1cccc2cccc1-2)/c1ccc2c(/C=C/n3cccc4cccc3-4)nnc-2cn1] | V-1267 |
| 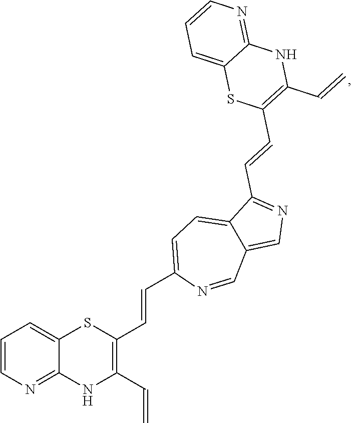 [SMILES: C=CC1=C(/C=C/c2ccc3c(/C=C/C4=C(C=C)Nc5ncccc5S4)ncc-3cn2)Sc2cccnc2N1] | V-1268 |
| 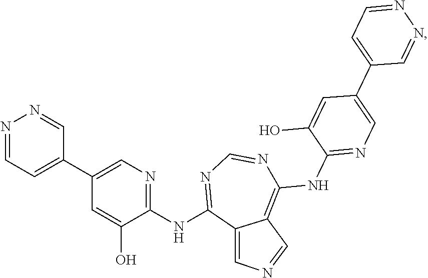 [SMILES: Oc1cc(-c2ccnnc2)cnc1Nc1ncnc(Nc2ncc(-c3ccnnc3)cc2O)c2cncc1-2] | V-1269 |
| 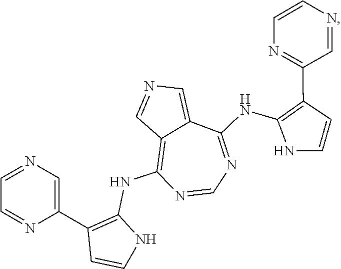 [SMILES: c1cnc(-c2cc[nH]c2Nc2ncnc(Nc3[nH]ccc3-c3cnccn3)c3cncc2-3)cn1] | V-1270 |
| 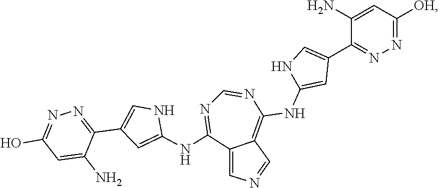 [SMILES: Nc1cc(O)nnc1-c1c[nH]c(Nc2ncnc(Nc3cc(-c4nnc(O)cc4N)c[nH]3)c3cncc2-3)c1] | V-1271 |
| 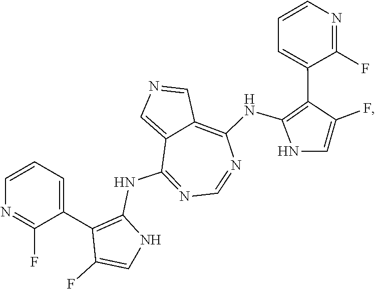 [SMILES: Fc1c[nH]c(Nc2ncnc(Nc3[nH]cc(F)c3-c3cccnc3F)c3cncc2-3)c1-c1cccnc1F] | V-1272 |
| 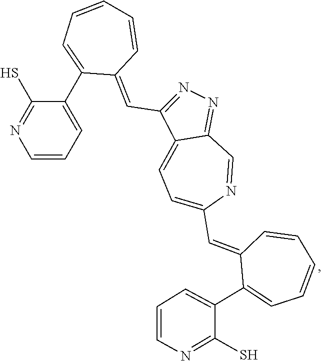 [SMILES: Sc1ncccc1C1=CC=CC=C/C1=C\c1ccc2c(/C=C3\C=CC=CC=C3c3cccnc3S)nnc-2cn1] | V-1273 |
| 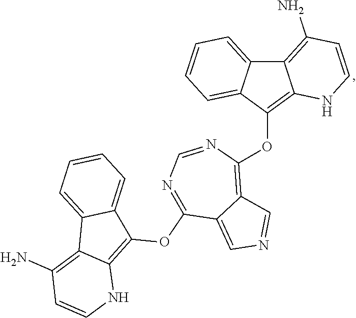 [SMILES: Nc1cc[nH]c2c(Oc3ncnc(Oc4c5[nH]ccc(N)c-5c5ccccc45)c4cncc3-4)c3ccccc3c1-2] | V-1274 |
| 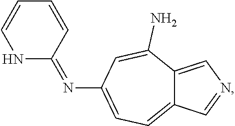 [SMILES: Nc1cc(/N=c2\cccc[nH]2)ccc2cncc1-2] | V-1275 |
| 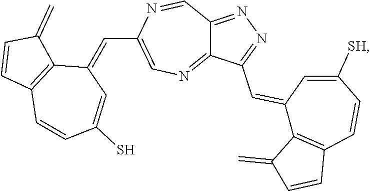 [SMILES: C=c1ccc2ccc(S)c/c(=C\c3cnc4c(/C=c5\cc(S)ccc6ccc(=C)c5=6)nnc-4cn3)c1=2] | V-1276 |
| 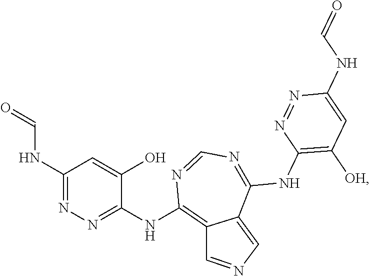 [SMILES: O=CNc1cc(O)c(Nc2ncnc(Nc3nnc(NC=O)cc3O)c3cncc2-3)nn1] | V-1277 |
| 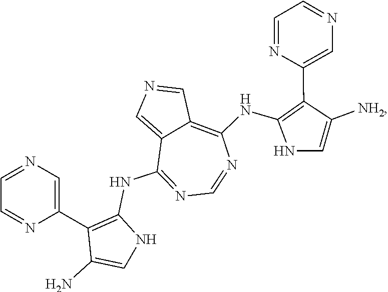 [SMILES: Nc1c[nH]c(Nc2ncnc(Nc3[nH]cc(N)c3-c3cnccn3)c3cncc2-3)c1-c1cnccn1] | V-1278 |
| 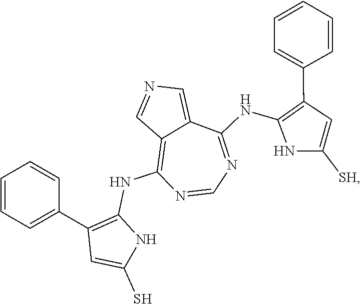 [SMILES: Sc1cc(-c2ccccc2)c(Nc2ncnc(Nc3[nH]c(S)cc3-c3ccccc3)c3cncc2-3)[nH]1] | V-1279 |
| 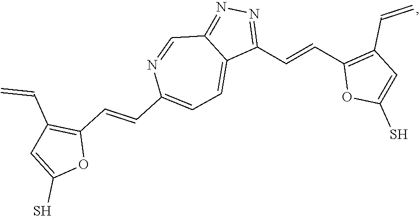 [SMILES: C=Cc1cc(S)oc1/C=C/c1ccc2c(/C=C/c3oc(S)cc3C=C)nnc-2cn1] | V-1280 |
| 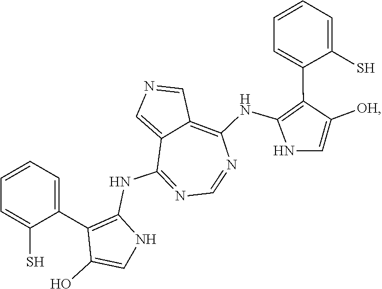 [SMILES: Oc1c[nH]c(Nc2ncnc(Nc3[nH]cc(O)c3-c3ccccc3S)c3cncc2-3)c1-c1ccccc1S] | V-1281 |
| 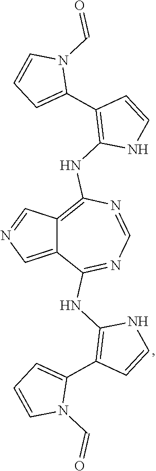 [SMILES: O=Cn1cccc1-c1cc[nH]c1Nc1ncnc(Nc2[nH]ccc2-c2cccn2C=O)c2cncc1-2] | V-1282 |
| 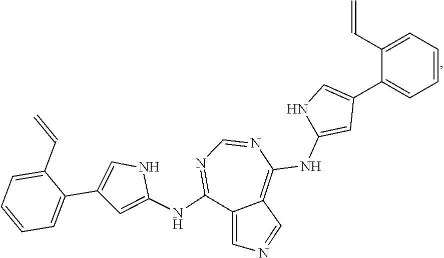 [SMILES: C=Cc1ccccc1-c1c[nH]c(Nc2ncnc(Nc3cc(-c4ccccc4C=C)c[nH]3)c3cncc2-3)c1] | V-1283 |
| 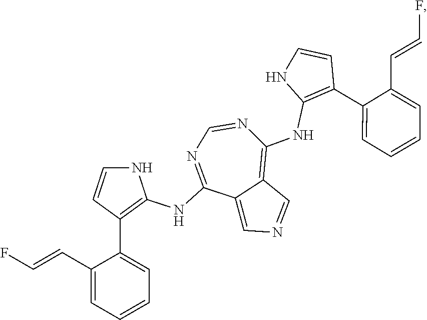 [SMILES: F/C=C/c1ccccc1-c1cc[nH]c1Nc1ncnc(Nc2[nH]ccc2-c2ccccc2/C=C/F)c2cncc1-2] | V-1284 |
| 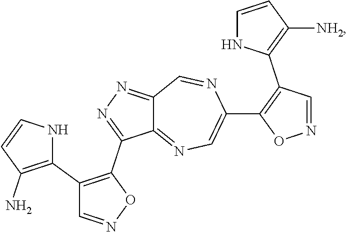 [SMILES: Nc1cc[nH]c1-c1cnoc1-c1cnc2c(-c3oncc3-c3[nH]ccc3N)nnc-2cn1] | V-1285 |
| 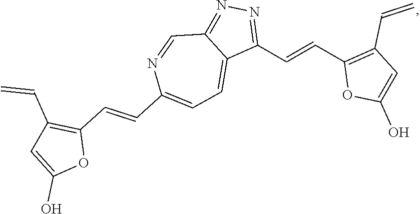 [SMILES: C=Cc1cc(O)oc1/C=C/c1ccc2c(/C=C/c3oc(O)cc3C=C)nnc-2cn1] | V-1286 |
| 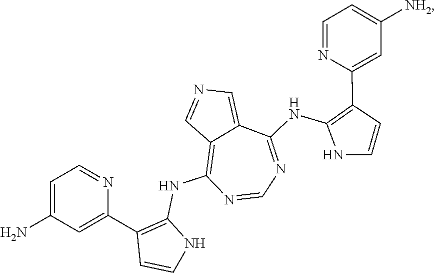 [SMILES: Nc1ccnc(-c2cc[nH]c2Nc2ncnc(Nc3[nH]ccc3-c3cc(N)ccn3)c3cncc2-3)c1] | V-1287 |
| 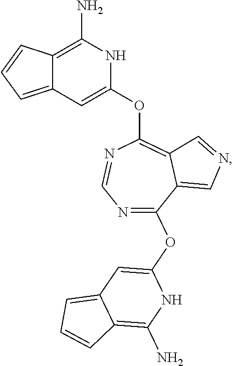 [SMILES: Nc1[nH]c(Oc2ncnc(Oc3cc4cccc-4c(N)[nH]3)c3cncc2-3)cc2cccc1-2] | V-1288 |
| 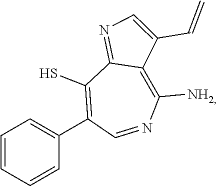 [SMILES: C=Cc1cnc2c(S)c(-c3ccccc3)cnc(N)c1-2] | V-1289 |
| 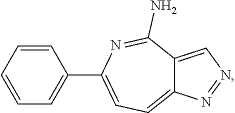 [SMILES: Nc1nc(-c2ccccc2)ccc2nncc1-2] | V-1290 |
| 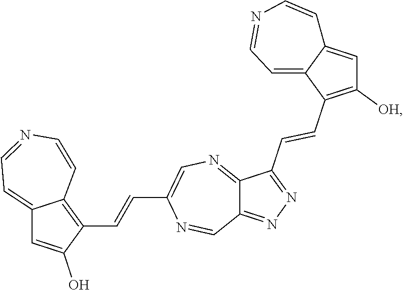 [SMILES: Oc1cc2ccnccc-2c1/C=C/c1cnc2c(/C=C/c3c(O)cc4ccnccc3-4)nnc-2cn1] | V-1291 |
| 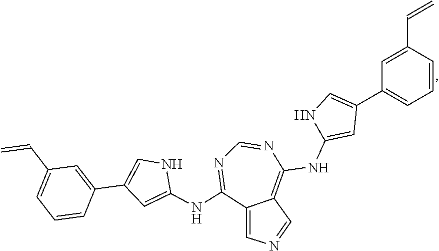 [SMILES: C=Cc1cccc(-c2c[nH]c(Nc3ncnc(Nc4cc(-c5cccc(C=C)c5)c[nH]4)c4cncc3-4)c2)c1] | V-1292 |
| 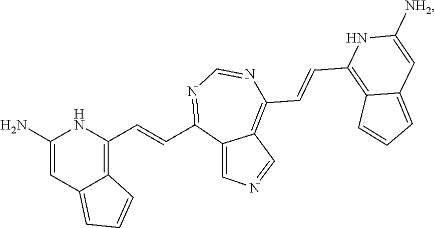 [SMILES: Nc1cc2cccc-2c(/C=C/c2ncnc(/C=C/c3[nH]c(N)cc4cccc3-4)c3cncc2-3)[nH]1] | V-1293 |
| 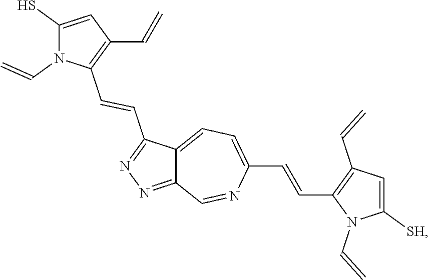 [SMILES: C=Cc1cc(S)n(C=C)c1/C=C/c1ccc2c(/C=C/c3c(C=C)cc(S)n3C=C)nnc-2cn1] | V-1294 |
| 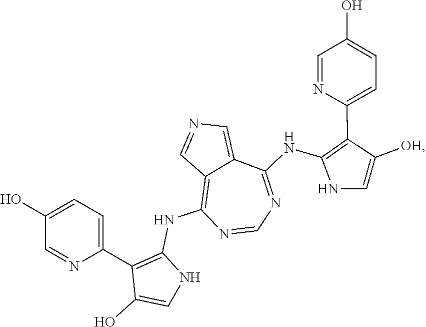 [SMILES: Oc1ccc(-c2c(O)c[nH]c2Nc2ncnc(Nc3[nH]cc(O)c3-c3ccc(O)cn3)c3cncc2-3)nc1] | V-1295 |
| 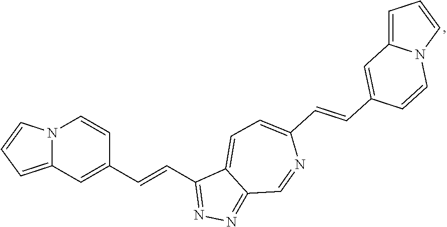 [SMILES: C(=C\c1ccc2c(/C=C/c3ccn4cccc4c3)nnc-2cn1)/c1ccn2cccc2c1] | V-1296 |
| 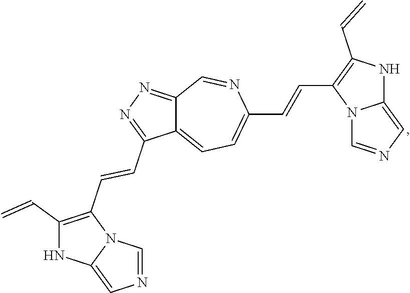 [SMILES: C=Cc1[nH]c2cncn2c1/C=C/c1ccc2c(/C=C/c3c(C=C)[nH]c4cncn34)nnc-2cn1] | V-1297 |
| 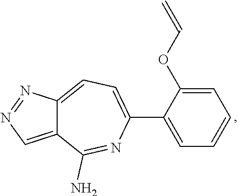 [SMILES: C=COc1ccccc1-c1ccc2nncc-2c(N)n1] | V-1298 |
| 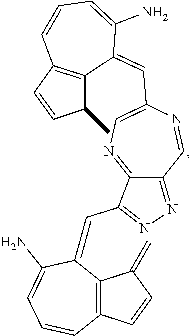 [SMILES: C=c1ccc2cccc(N)/c(=C/c3nnc4cnc(/C=C5/C(N)=CC=CC6=C5[C@@H](C)C=C6)cnc3-4)c1=2] | V-1299 |
| 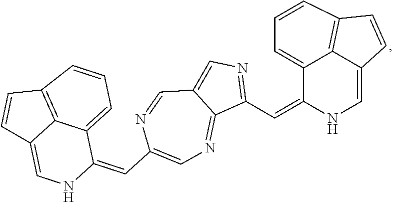 [SMILES: C(/c1cnc2c(/C=c3/[nH]cc4ccc5cccc3c5-4)ncc-2cn1)=c1\[nH]cc2ccc3cccc1c3-2] | V-1300 |
| 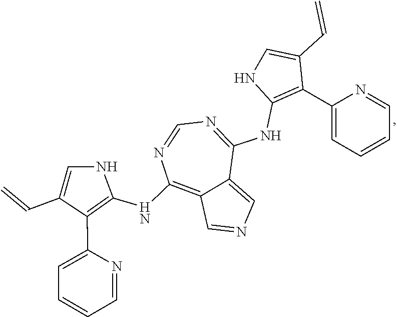 [SMILES: C=Cc1c[nH]c(Nc2ncnc(Nc3[nH]cc(C=C)c3-c3ccccn3)c3cncc2-3)c1-c1ccccn1] | V-1301 |
| 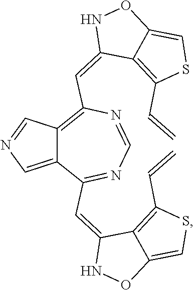 [SMILES: C=Cc1scc2c1/C(=C\c1ncnc(/C=C3/NOc4csc(C=C)c43)c3cncc1-3)NO2] | V-1302 |
| 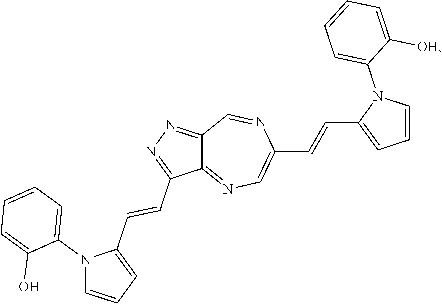 [SMILES: Oc1ccccc1-n1cccc1/C=C/c1cnc2c(/C=C/c3cccn3-c3ccccc3O)nnc-2cn1] | V-1303 |
| 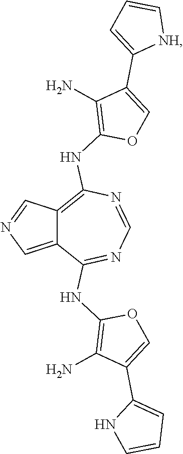 [SMILES: Nc1c(-c2ccc[nH]2)coc1Nc1ncnc(Nc2occ(-c3ccc[nH]3)c2N)c2cncc1-2] | V-1304 |
| 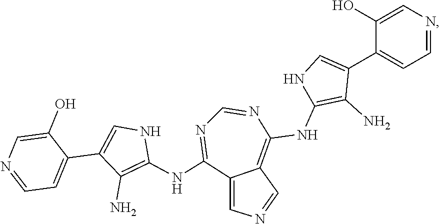 [SMILES: Nc1c(-c2ccncc2O)c[nH]c1Nc1ncnc(Nc2[nH]cc(-c3ccncc3O)c2N)c2cncc1-2] | V-1305 |
| 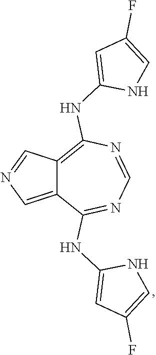 [SMILES: Fc1c[nH]c(Nc2ncnc(Nc3cc(F)c[nH]3)c3cncc2-3)c1] | V-1306 |
| 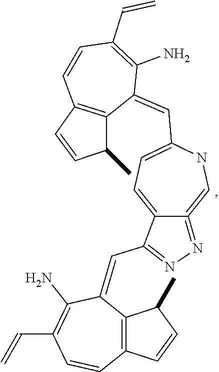 | V-1307 |
| 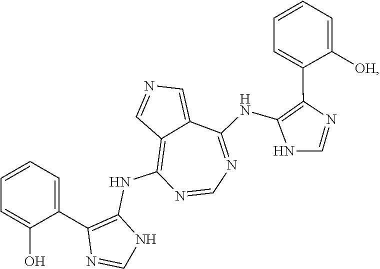 [SMILES: Oc1ccccc1-c1nc[nH]c1Nc1ncnc(Nc2[nH]cnc2-c2ccccc2O)c2cncc1-2] | V-1308 |
| 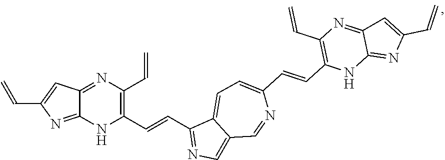 [SMILES: C=Cc1cc2nc(C=C)c(/C=C/c3ccc4c(/C=C/c5[nH]c6nc(C=C)cc-6nc5C=C)ncc-4cn3)[nH]c-2n1] | V-1309 |
| 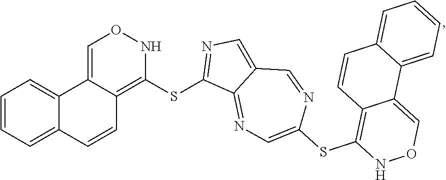 [SMILES: C1=c2c(ccc3ccccc23)=C(Sc2cnc3c(SC4=c5ccc6ccccc6c5=CON4)ncc-3cn2)NO1] | V-1310 |
| 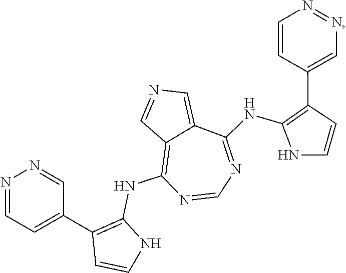 [SMILES: c1cc(-c2cc[nH]c2Nc2ncnc(Nc3[nH]ccc3-c3ccnnc3)c3cncc2-3)cnn1] | V-1311 |
| 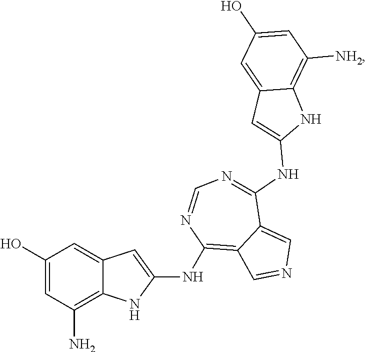 [SMILES: Nc1cc(O)cc2cc(Nc3ncnc(Nc4cc5cc(O)cc(N)c5[nH]4)c4cncc3-4)[nH]c12] | V-1312 |
| 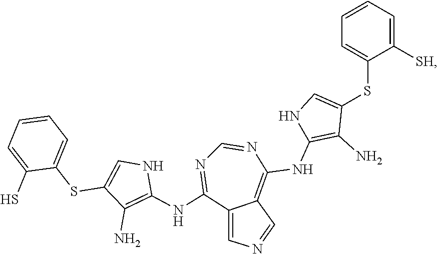 [SMILES: Nc1c(Sc2ccccc2S)c[nH]c1Nc1ncnc(Nc2[nH]cc(Sc3ccccc3S)c2N)c2cncc1-2] | V-1313 |
| 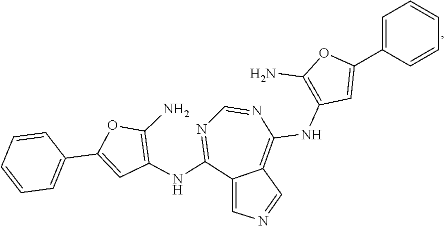 [SMILES: Nc1oc(-c2ccccc2)cc1Nc1ncnc(Nc2cc(-c3ccccc3)oc2N)c2cncc1-2] | V-1314 |
| 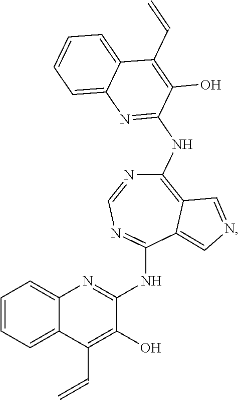 [SMILES: C=Cc1c(O)c(Nc2ncnc(Nc3nc4ccccc4c(C=C)c3O)c3cncc2-3)nc2ccccc12] | V-1315 |
| 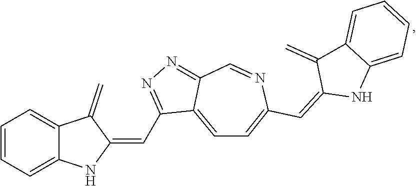 [SMILES: C=c1/c(=C\c2ccc3c(/C=c4/[nH]c5ccccc5c4=C)nnc-3cn2)[nH]c2ccccc12] | V-1316 |
| 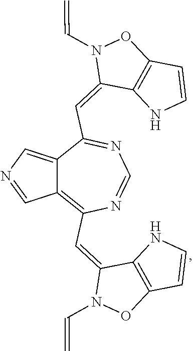 [SMILES: C=CN1Oc2cc[nH]c2/C1=C\c1ncnc(/C=C2\c3[nH]ccc3ON2C=C)c2cncc1-2] | V-1317 |
| 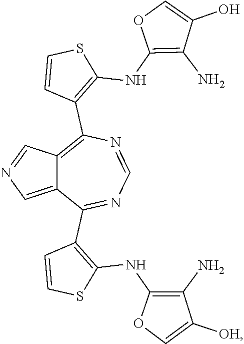 [SMILES: Nc1c(O)coc1Nc1sccc1-c1ncnc(-c2ccsc2Nc2occ(O)c2N)c2cncc1-2] | V-1318 |
| 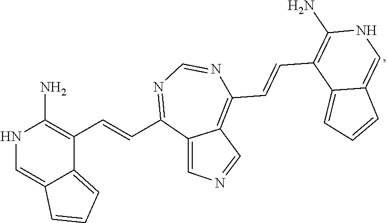 [SMILES: Nc1[nH]cc2cccc-2c1/C=C/c1ncnc(/C=C/c2c3cccc-3c[nH]c2N)c2cncc1-2] | V-1319 |
| 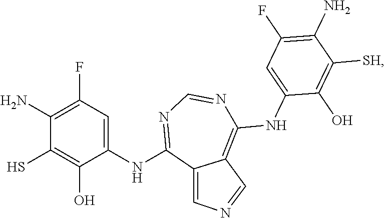 [SMILES: Nc1c(F)cc(Nc2ncnc(Nc3cc(F)c(N)c(S)c3O)c3cncc2-3)c(O)c1S] | V-1320 |
| 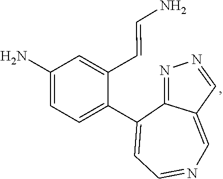 [SMILES: N/C=C/c1cc(N)ccc1-c1ccncc2cnnc1-2] | V-1321 |
| 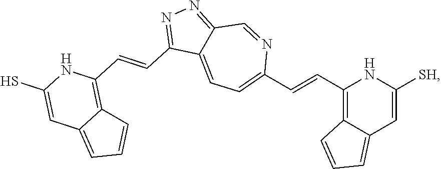 [SMILES: Sc1cc2cccc-2c(/C=C/c2ccc3c(/C=C/c4[nH]c(S)cc5cccc4-5)nnc-3cn2)[nH]1] | V-1322 |
| 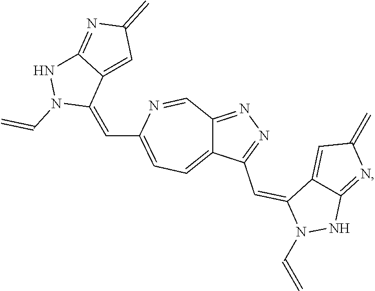 [SMILES: C=Cn1[nH]c2nc(=C)cc-2/c1=C\c1ccc2c(/C=c3\c4cc(=C)nc-4[nH]n3C=C)nnc-2cn1] | V-1323 |
| 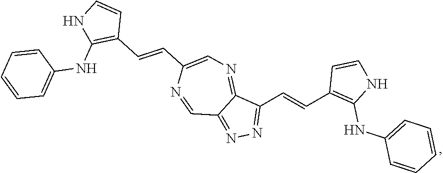 [SMILES: C(=C\c1cc[nH]c1Nc1ccccc1)/c1cnc2c(/C=C/c3cc[nH]c3Nc3ccccc3)nnc-2cn1] | V-1324 |
| 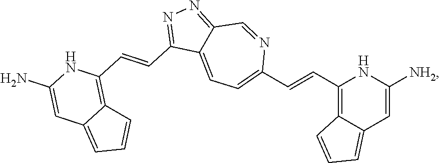 [SMILES: Nc1cc2cccc-2c(/C=C/c2ccc3c(/C=C/c4[nH]c(N)cc5cccc4-5)nnc-3cn2)[nH]1] | V-1325 |
| 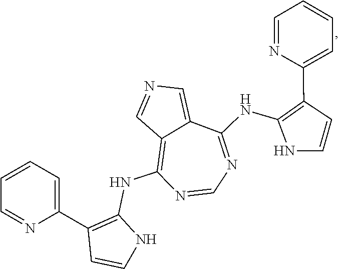 [SMILES: c1ccc(-c2cc[nH]c2Nc2ncnc(Nc3[nH]ccc3-c3ccccn3)c3cncc2-3)nc1] | V-1326 |
| 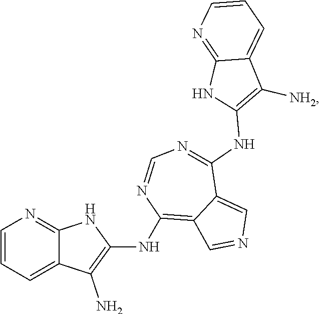 [SMILES: Nc1c(Nc2ncnc(Nc3[nH]c4ncccc4c3N)c3cncc2-3)[nH]c2ncccc12] | V-1327 |
| 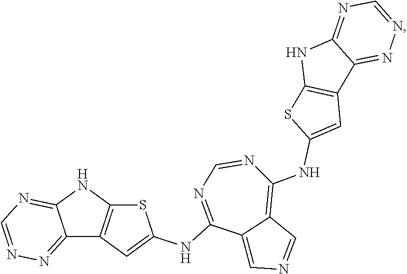 [SMILES: c1nc(Nc2cc3c([nH]c4ncnnc43)s2)c2cncc-2c(Nc2cc3c([nH]c4ncnnc43)s2)n1] | V-1328 |
| 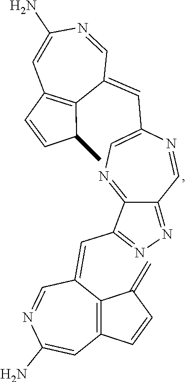 [SMILES: C=c1ccc2cc(N)nc/c(=C/c3nnc4cnc(/C=C5/C=NC(N)=CC6=C5[C@@H](C)C=C6)cnc3-4)c1=2] | V-1329 |
| 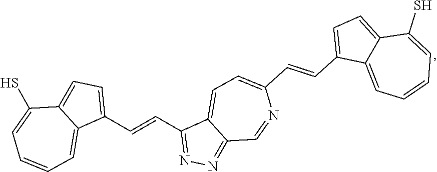 [SMILES: Sc1ccccc2c(/C=C/c3ccc4c(/C=C/c5ccc6c(S)ccccc5-6)nnc-4cn3)ccc1-2] | V-1330 |
| 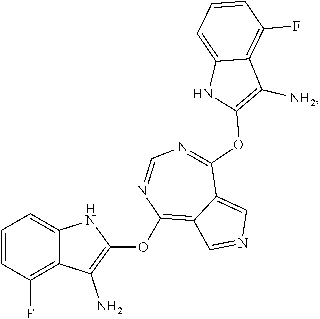 [SMILES: Nc1c(Oc2ncnc(Oc3[nH]c4cccc(F)c4c3N)c3cncc2-3)[nH]c2cccc(F)c12] | V-1331 |
| 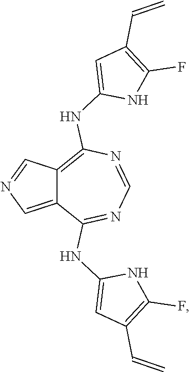 [SMILES: C=Cc1cc(Nc2ncnc(Nc3cc(C=C)c(F)[nH]3)c3cncc2-3)[nH]c1F] | V-1332 |
| 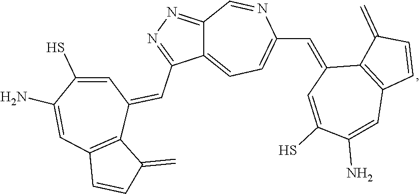 [SMILES: C=c1ccc2cc(N)c(S)c/c(=C\c3ccc4c(/C=c5\cc(S)c(N)cc6ccc(=C)c5=6)nnc-4cn3)c1=2] | V-1333 |
| 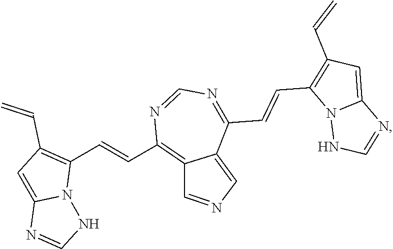 [SMILES: C=Cc1cc2nc[nH]n2c1/C=C/c1ncnc(/C=C/c2c(C=C)cc3nc[nH]n23)c2cncc1-2] | V-1334 |
| 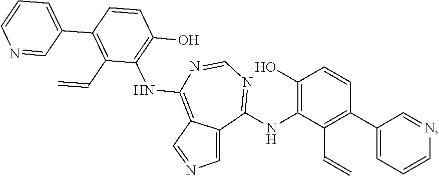 [SMILES: C=Cc1c(-c2cccnc2)ccc(O)c1Nc1ncnc(Nc2c(O)ccc(-c3cccnc3)c2C=C)c2cncc1-2] | V-1335 |
| 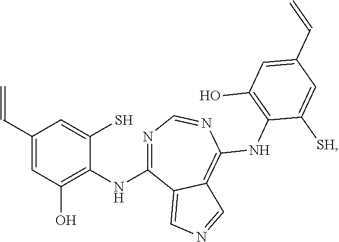 [SMILES: C=Cc1cc(O)c(Nc2ncnc(Nc3c(O)cc(C=C)cc3S)c3cncc2-3)c(S)c1] | V-1336 |
| 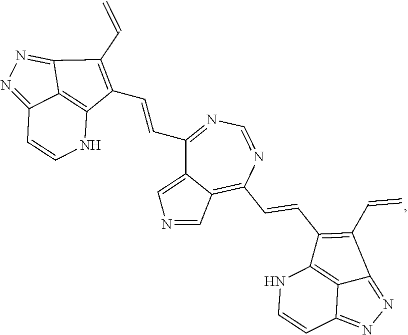 [SMILES: C=Cc1c(/C=C/c2ncnc(/C=C/c3c(C=C)c4nnc5cc[nH]c3c54)c3cncc2-3)c2[nH]ccc3nnc1c32] | V-1337 |
| 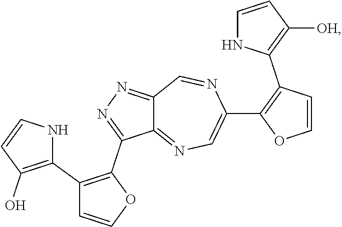 [SMILES: Oc1cc[nH]c1-c1ccoc1-c1cnc2c(-c3occc3-c3[nH]ccc3O)nnc-2cn1] | V-1338 |
| 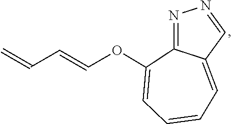 | V-1339 |
| 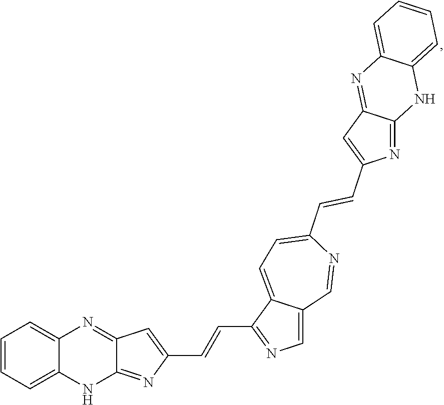 [SMILES: C(=C\c1cc2nc3ccccc3[nH]c-2n1)/c1ccc2c(/C=C/c3cc4nc5ccccc5[nH]c-4n3)ncc-2cn1] | V-1340 |
| 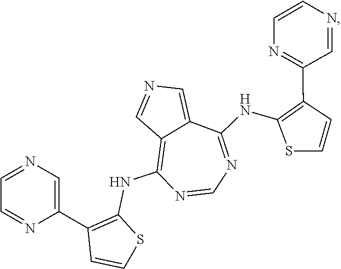 [SMILES: c1cnc(-c2ccsc2Nc2ncnc(Nc3sccc3-c3cnccn3)c3cncc2-3)cn1] | V-1341 |
| 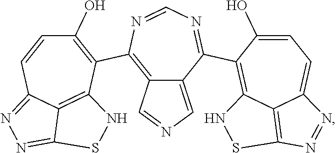 [SMILES: Oc1ccc2nnc3s[nH]c(c1-c1ncnc(-c4c(O)ccc5nnc6s[nH]c4c56)c4cncc1-4)c23] | V-1342 |
| 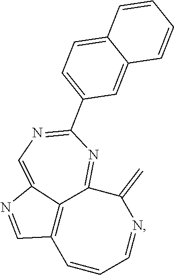 [SMILES: C=c1ncccc2cnc3cnc(-c4ccc5ccccc5c4)nc1c2-3] | V-1343 |
| 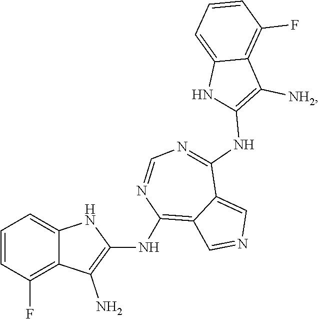 [SMILES: Nc1c(Nc2ncnc(Nc3[nH]c4cccc(F)c4c3N)c3cncc2-3)[nH]c2cccc(F)c12] | V-1344 |
| 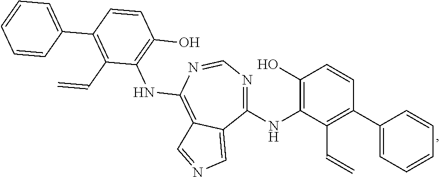 [SMILES: C=Cc1c(-c2ccccc2)ccc(O)c1Nc1ncnc(Nc2c(O)ccc(-c3ccccc3)c2C=C)c2cncc1-2] | V-1345 |
| 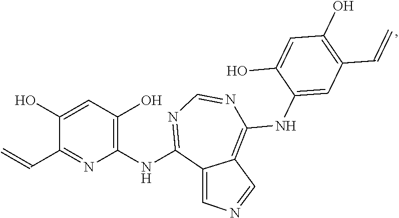 [SMILES: C=Cc1cc(Nc2ncnc(Nc3nc(C=C)c(O)cc3O)c3cncc2-3)c(O)cc1O] | V-1346 |
| 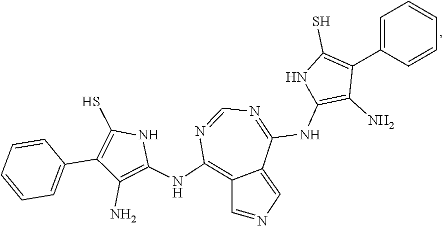 [SMILES: Nc1c(Nc2ncnc(Nc3[nH]c(S)c(-c4ccccc4)c3N)c3cncc2-3)[nH]c(S)c1-c1ccccc1] | V-1347 |
| 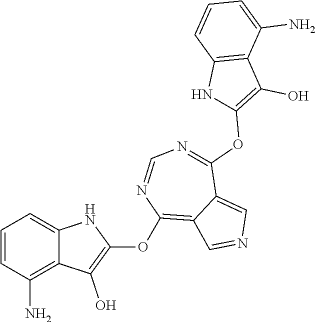 [SMILES: Nc1cccc2[nH]c(Oc3ncnc(Oc4[nH]c5cccc(N)c5c4O)c4cncc3-4)c(O)c12] | V-1348 |
| 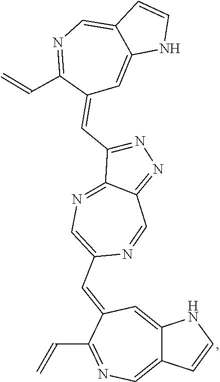 [SMILES: C=CC1=NC=c2cc[nH]c2=C/C1=C\c1cnc2c(/C=C3\C=c4[nH]ccc4=CN=C3C=C)nnc-2cn1] | V-1349 |
| 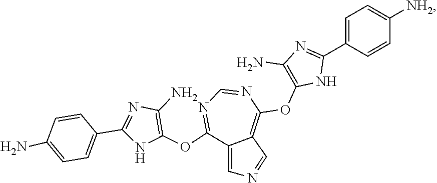 [SMILES: Nc1ccc(-c2nc(N)c(Oc3ncnc(Oc4[nH]c(-c5ccc(N)cc5)nc4N)c4cncc3-4)[nH]2)cc1] | V-1350 |
| 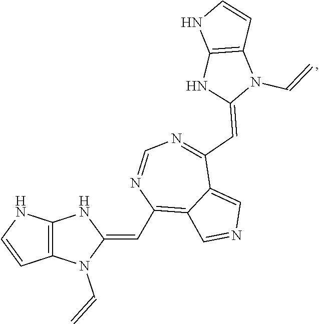 [SMILES: C=CN1/C(=C/c2ncnc(/C=C3\Nc4[nH]ccc4N3C=C)c3cncc2-3)Nc2[nH]ccc21] | V-1351 |
| 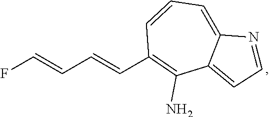 [SMILES: Nc1c(/C=C/C=C/F)cccc2nccc1-2] | V-1352 |
| 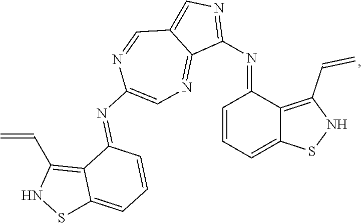 [SMILES: C=Cc1[nH]sc2ccc/c(=N\c3cnc4c(/N=c5\cccc6s[nH]c(C=C)c5-6)ncc-4cn3)c1-2] | V-1353 |
| 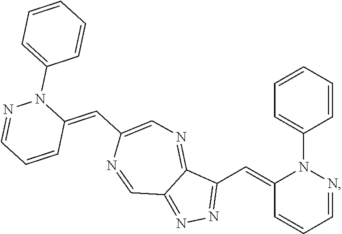 [SMILES: C1=C/C(=C\c2cnc3c(/C=C4\C=CC=NN4c4ccccc4)nnc-3cn2)N(c2ccccc2)N=C1] | V-1354 |
| 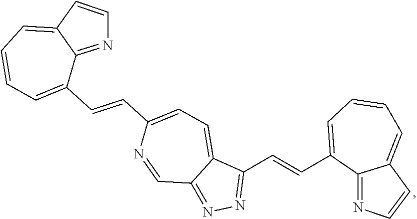 [SMILES: C(=C\c1ccccc2ccnc1-2)/c1ccc2c(/C=C/c3ccccc4ccnc3-4)nnc-2cn1] | V-1355 |
| 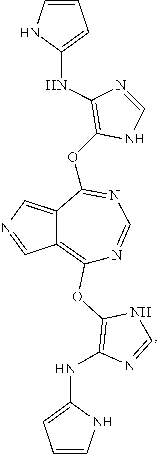 [SMILES: c1c[nH]c(Nc2nc[nH]c2Oc2ncnc(Oc3[nH]cnc3Nc3ccc[nH]3)c3cncc2-3)c1] | V-1356 |
| 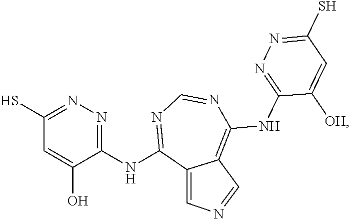 [SMILES: Oc1cc(S)nnc1Nc1ncnc(Nc2nnc(S)cc2O)c2cncc1-2] | V-1357 |
| 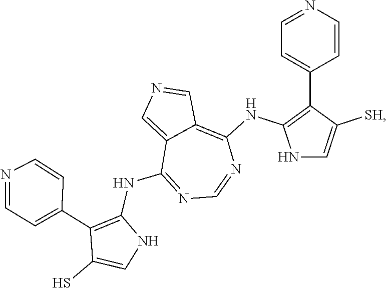 [SMILES: Sc1c[nH]c(Nc2ncnc(Nc3[nH]cc(S)c3-c3ccncc3)c3cncc2-3)c1-c1ccncc1] | V-1358 |
| 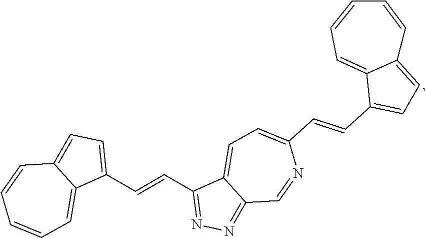 [SMILES: C(=C\c1ccc2cccccc1-2)/c1ccc2c(/C=C/c3ccc4cccccc3-4)nnc-2cn1] | V-1359 |
| 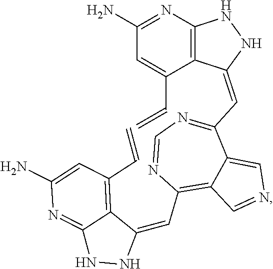 [SMILES: C=Cc1cc(N)nc2c1/C(=C\c1ncnc(/C=C3/NNc4nc(N)cc(C=C)c43)c3cncc1-3)NN2] | V-1360 |
| 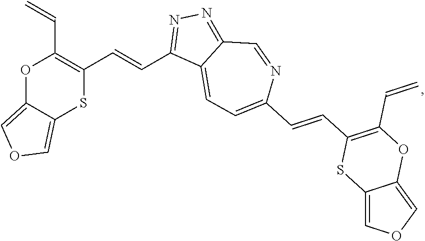 [SMILES: C=CC1=C(/C=C/c2ccc3c(/C=C/C4=C(C=C)Oc5cocc5S4)nnc-3cn2)Sc2cocc2O1] | V-1361 |
| 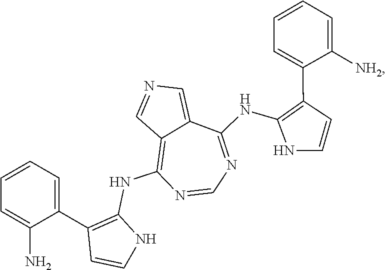 [SMILES: Nc1ccccc1-c1cc[nH]c1Nc1ncnc(Nc2[nH]ccc2-c2ccccc2N)c2cncc1-2] | V-1362 |
| 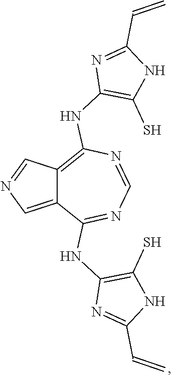 [SMILES: C=Cc1nc(Nc2ncnc(Nc3nc(C=C)[nH]c3S)c3cncc2-3)c(S)[nH]1] | V-1363 |
| 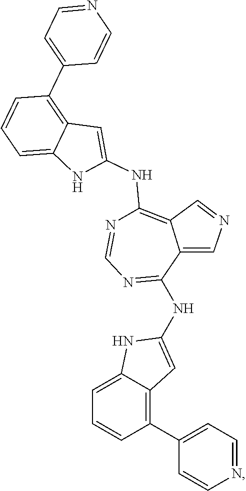 [SMILES: c1cc(-c2ccncc2)c2cc(Nc3ncnc(Nc4cc5c(-c6ccncc6)cccc5[nH]4)c4cncc3-4)[nH]c2c1] | V-1364 |
| 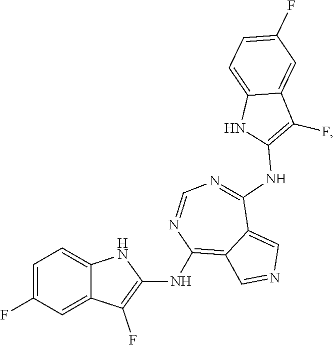 [SMILES: Fc1ccc2[nH]c(Nc3ncnc(Nc4[nH]c5ccc(F)cc5c4F)c4cncc3-4)c(F)c2c1] | V-1365 |
| 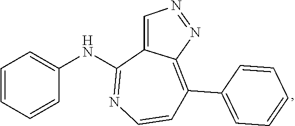 [SMILES: c1ccc(Nc2nccc(-c3ccccc3)c3nncc2-3)cc1] | V-1366 |
| 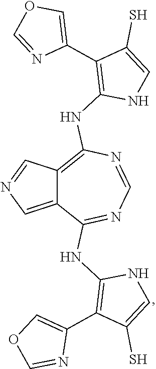 [SMILES: Sc1c[nH]c(Nc2ncnc(Nc3[nH]cc(S)c3-c3cocn3)c3cncc2-3)c1-c1cocn1] | V-1367 |
| 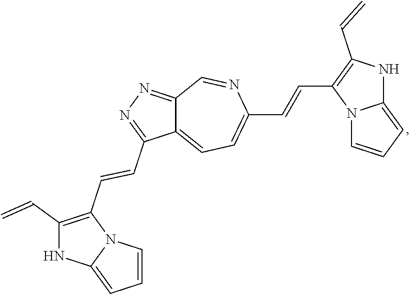 [SMILES: C=Cc1[nH]c2cccn2c1/C=C/c1ccc2c(/C=C/c3c(C=C)[nH]c4cccn34)nnc-2cn1] | V-1368 |
| 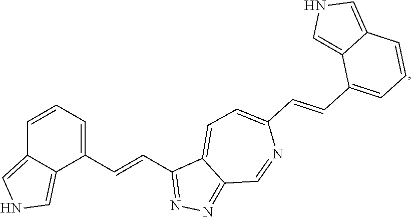 [SMILES: C(=C\c1cccc2c[nH]cc12)/c1ccc2c(/C=C/c3cccc4c[nH]cc34)nnc-2cn1] | V-1369 |
| 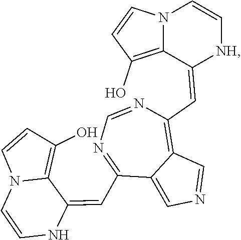 [SMILES: Oc1ccn2c1/C(=C\c1ncnc(/C=C3/NC=Cn4ccc(O)c43)c3cncc1-3)NC=C2] | V-1370 |
| 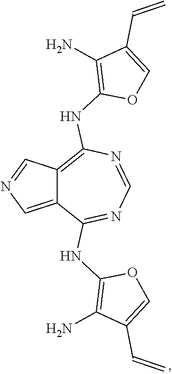 [SMILES: C=Cc1coc(Nc2ncnc(Nc3occ(C=C)c3N)c3cncc2-3)c1N] | V-1371 |
| 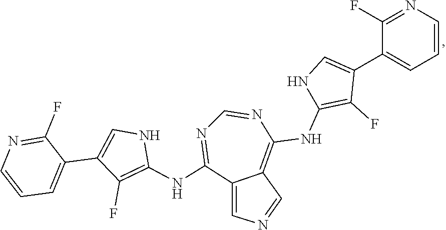 [SMILES: Fc1ncccc1-c1c[nH]c(Nc2ncnc(Nc3[nH]cc(-c4cccnc4F)c3F)c3cncc2-3)c1F] | V-1372 |
| 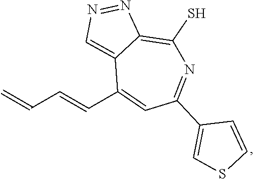 [SMILES: C=C/C=C/c1cc(-c2ccsc2)nc(S)c2nncc1-2] | V-1373 |
| 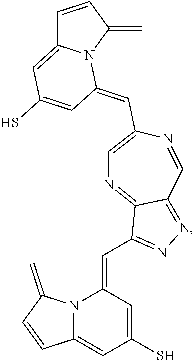 [SMILES: C=c1ccc2cc(S)c/c(=C\c3cnc4c(/C=c5\cc(S)cc6ccc(=C)n56)nnc-4cn3)n12] | V-1374 |
| 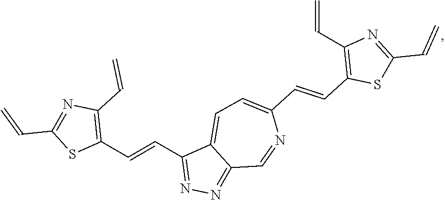 [SMILES: C=Cc1nc(C=C)c(/C=C/c2ccc3c(/C=C/c4sc(C=C)nc4C=C)nnc-3cn2)s1] | V-1375 |
| 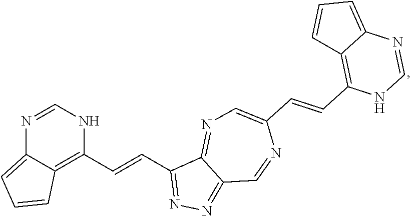 [SMILES: C(=C\c1[nH]cnc2cccc1-2)/c1cnc2c(/C=C/c3[nH]cnc4cccc3-4)nnc-2cn1] | V-1376 |
| 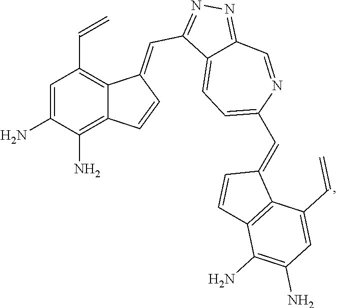 [SMILES: C=Cc1cc(N)c(N)c2c1/C(=C/c1ccc3c(/C=C4\C=Cc5c(N)c(N)cc(C=C)c54)nnc-3cn1)C=C2] | V-1377 |
| 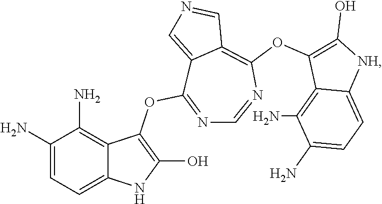 [SMILES: Nc1ccc2[nH]c(O)c(Oc3ncnc(Oc4c(O)[nH]c5ccc(N)c(N)c45)c4cncc3-4)c2c1N] | V-1378 |
| 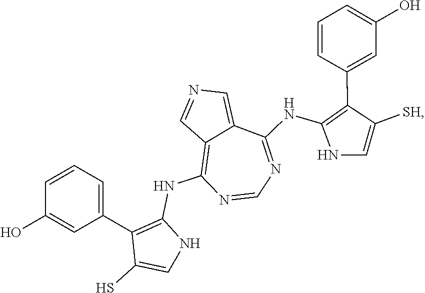 [SMILES: Oc1cccc(-c2c(S)c[nH]c2Nc2ncnc(Nc3[nH]cc(S)c3-c3cccc(O)c3)c3cncc2-3)c1] | V-1379 |
| 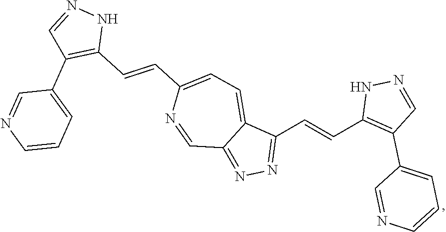 [SMILES: C(=C\c1[nH]ncc1-c1cccnc1)/c1ccc2c(/C=C/c3[nH]ncc3-c3cccnc3)nnc-2cn1] | V-1380 |
| 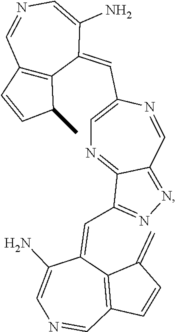 [SMILES: C=c1ccc2cncc(N)/c(=C/c3nnc4cnc(/C=C5/C(N)=CN=CC6=C5[C@@H](C)C=C6)cnc3-4)c1=2] | V-1381 |
| 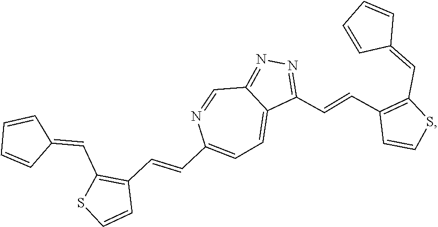 [SMILES: C1=CC(=Cc2sccc2/C=C/c2ccc3c(/C=C/c4ccsc4C=C4C=CC=C4)nnc-3cn2)C=C1] | V-1382 |
| 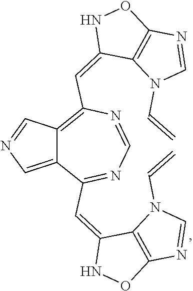 [SMILES: C=Cn1cnc2c1/C(=C\c1ncnc(/C=C3/NOc4ncn(C=C)c43)c3cncc1-3)NO2] | V-1383 |
| 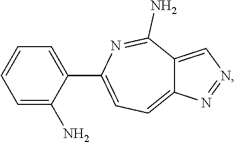 [SMILES: Nc1ccccc1-c1ccc2nncc-2c(N)n1] | V-1384 |
| 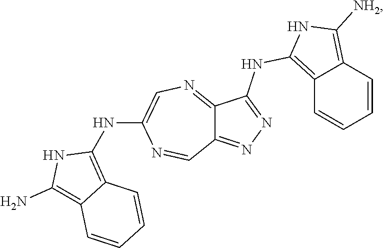 [SMILES: Nc1[nH]c(Nc2cnc3c(Nc4[nH]c(N)c5ccccc45)nnc-3cn2)c2ccccc12] | V-1385 |
| 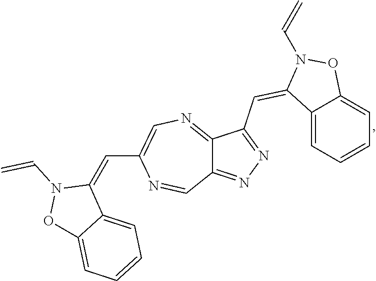 [SMILES: C=CN1Oc2ccccc2/C1=C\c1cnc2c(/C=C3\c4ccccc4ON3C=C)nnc-2cn1] | V-1386 |
| 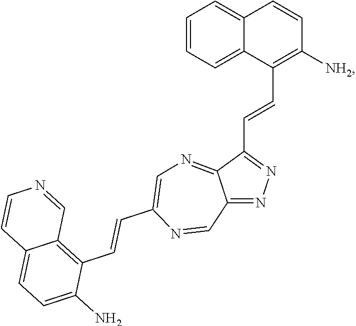 [SMILES: Nc1ccc2ccccc2c1/C=C/c1nnc2cnc(/C=C/c3c(N)ccc4ccncc34)cnc1-2] | V-1387 |
| 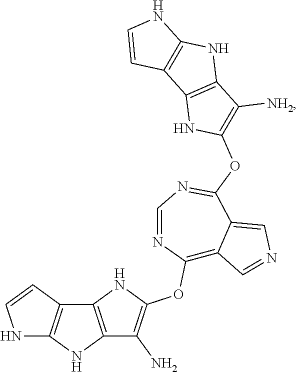 [SMILES: Nc1c(Oc2ncnc(Oc3[nH]c4c([nH]c5[nH]ccc54)c3N)c3cncc2-3)[nH]c2c1[nH]c1[nH]ccc12] | V-1388 |
| 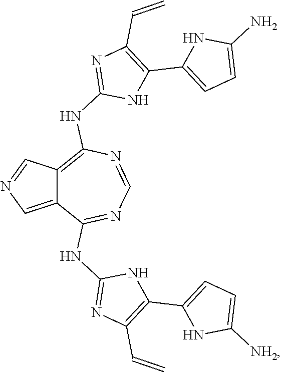 [SMILES: C=Cc1nc(Nc2ncnc(Nc3nc(C=C)c(-c4ccc(N)[nH]4)[nH]3)c3cncc2-3)[nH]c1-c1ccc(N)[nH]1] | V-1389 |
| 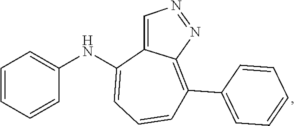 [SMILES: c1ccc(Nc2cccc(-c3ccccc3)c3nncc2-3)cc1] | V-1390 |
| 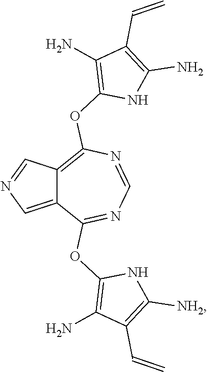 [SMILES: C=Cc1c(N)[nH]c(Oc2ncnc(Oc3[nH]c(N)c(C=C)c3N)c3cncc2-3)c1N] | V-1391 |
| 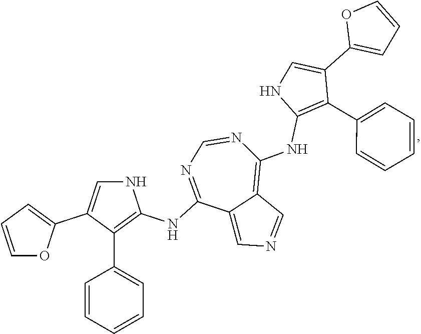 [SMILES: c1ccc(-c2c(-c3ccco3)c[nH]c2Nc2ncnc(Nc3[nH]cc(-c4ccco4)c3-c3ccccc3)c3cncc2-3)cc1] | V-1392 |
| 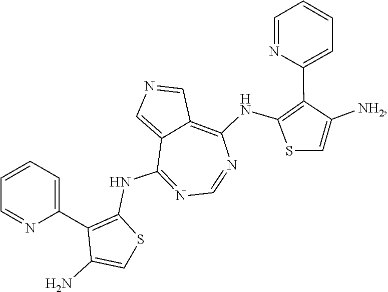 [SMILES: Nc1csc(Nc2ncnc(Nc3scc(N)c3-c3ccccn3)c3cncc2-3)c1-c1ccccn1] | V-1393 |
| 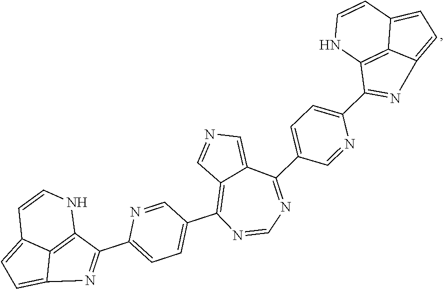 [SMILES: c1nc(-c2ccc(-c3nc4ccc5cc[nH]c3c54)nc2)c2cncc-2c(-c2ccc(-c3nc4ccc5cc[nH]c3c54)nc2)n1] | V-1394 |
| 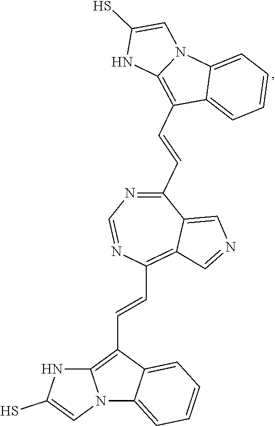 [SMILES: Sc1cn2c([nH]1)c(/C=C/c1ncnc(/C=C/c3c4ccccc4n4cc(S)[nH]c34)c3cncc1-3)c1ccccc12] | V-1395 |
| 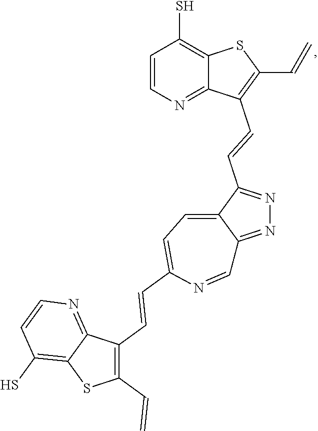 [SMILES: C=Cc1sc2c(S)ccnc2c1/C=C/c1ccc2c(/C=C/c3c(C=C)sc4c(S)ccnc34)nnc-2cn1] | V-1396 |
| 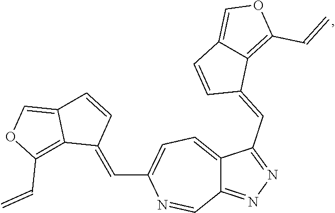 [SMILES: C=Cc1occ2c1/C(=C/c1ccc3c(/C=C4\C=Cc5coc(C=C)c54)nnc-3cn1)C=C2] | V-1397 |
| 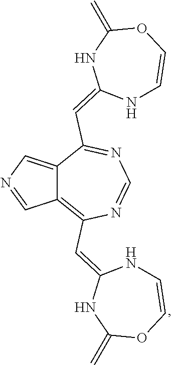 [SMILES: C=c1[nH]/c(=C/c2ncnc(/C=c3\[nH]ccoc(=C)[nH]3)c3cncc2-3)[nH]cco1] | V-1398 |
| 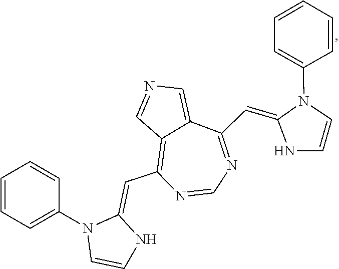 [SMILES: C1=CN(c2ccccc2)/C(=C/c2ncnc(/C=C3\NC=CN3c3ccccc3)c3cncc2-3)N1] | V-1399 |
| 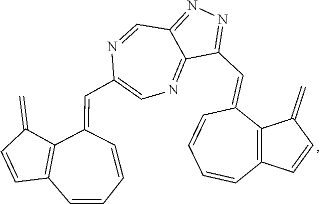 [SMILES: C=c1ccc2cccc/c(=C\c3cnc4c(/C=c5\ccccc6ccc(=C)c5=6)nnc-4cn3)c1=2] | V-1400 |
| 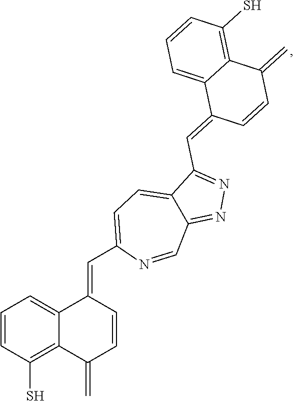 [SMILES: C=c1cc/c(=C\c2ccc3c(/C=c4\ccc(=C)c5c(S)cccc45)nnc-3cn2)c2cccc(S)c12] | V-1401 |
| 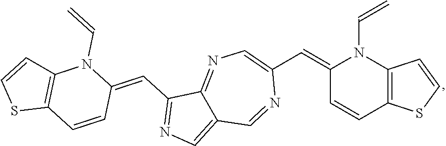 [SMILES: C=CN1/C(=C/c2cnc3c(/C=C4\C=Cc5sccc5N4C=C)ncc-3cn2)C=Cc2sccc21] | V-1402 |
| 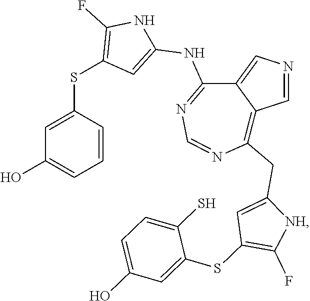 [SMILES: Oc1cccc(Sc2cc(Nc3ncnc(Cc4cc(Sc5cc(O)ccc5S)c(F)[nH]4)c4cncc3-4)[nH]c2F)c1] | V-1403 |
| 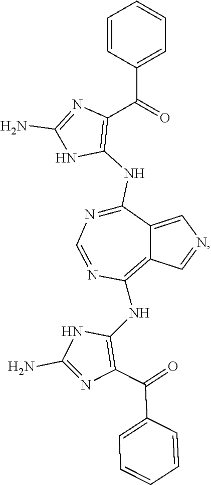 [SMILES: Nc1nc(C(=O)c2ccccc2)c(Nc2ncnc(Nc3[nH]c(N)nc3C(=O)c3ccccc3)c3cncc2-3)[nH]1] | V-1404 |
| 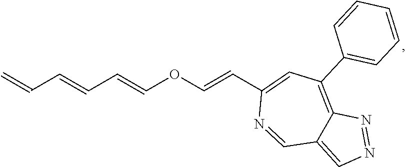 [SMILES: C=C/C=C/C=C/O/C=C/c1cc(-c2ccccc2)c2nncc-2cn1] | V-1405 |
| 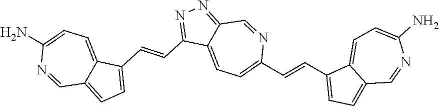 [SMILES: Nc1ccc2c(/C=C/c3ccc4c(/C=C/c5ccc6cnc(N)ccc5-6)nnc-4cn3)ccc-2cn1] | V-1406 |
| 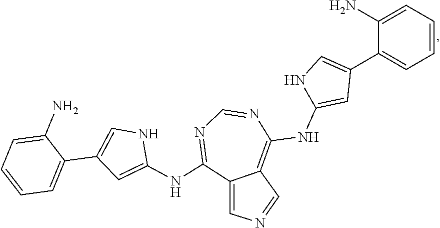 [SMILES: Nc1ccccc1-c1c[nH]c(Nc2ncnc(Nc3cc(-c4ccccc4N)c[nH]3)c3cncc2-3)c1] | V-1407 |
| 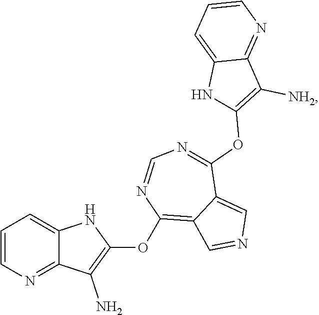 [SMILES: Nc1c(Oc2ncnc(Oc3[nH]c4cccnc4c3N)c3cncc2-3)[nH]c2cccnc12] | V-1408 |
| 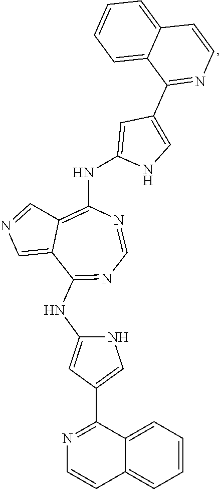 [SMILES: c1ccc2c(-c3c[nH]c(Nc4ncnc(Nc5cc(-c6nccc7ccccc67)c[nH]5)c5cncc4-5)c3)nccc2c1] | V-1409 |
| 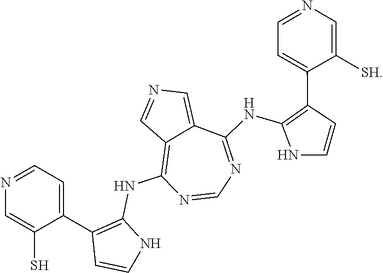 [SMILES: Sc1cnccc1-c1cc[nH]c1Nc1ncnc(Nc2[nH]ccc2-c2ccncc2S)c2cncc1-2] | V-1410 |

In some embodiments, the compound of Formula V comprises 1 to 6 substituents of Formula Va. In some embodiments, each L is a bond and each R¹ is independently selected from CN and NH₂. In some embodiments, the compound of Formula V is selected from compounds shown in Table B and salt and/or tautomer thereof.

TABLE B
Exemplary Compounds of Formula V

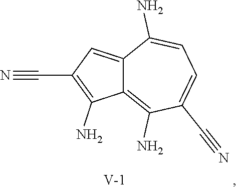

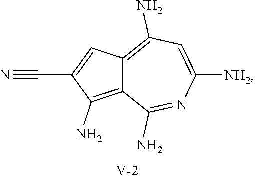

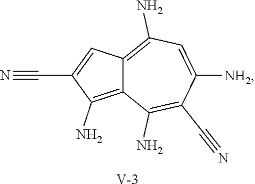

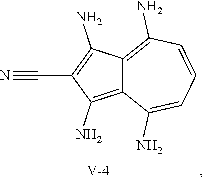

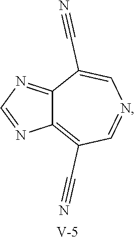

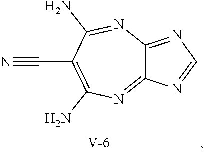

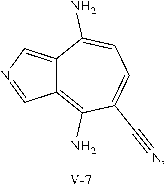

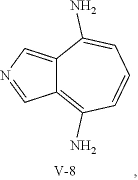

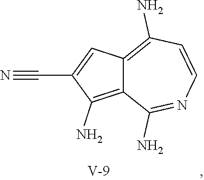

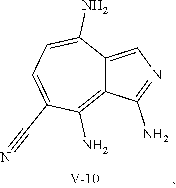

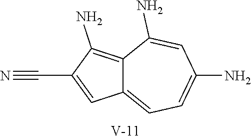

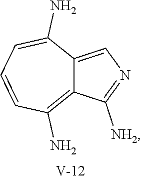

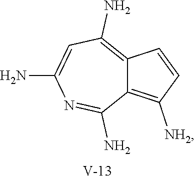

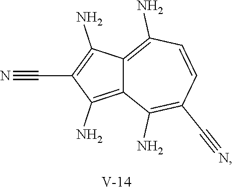

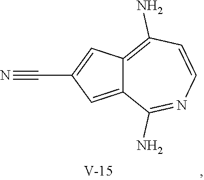

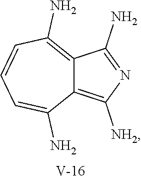

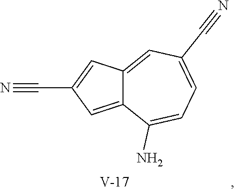

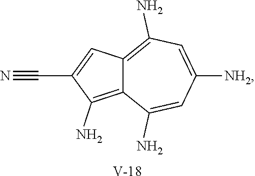

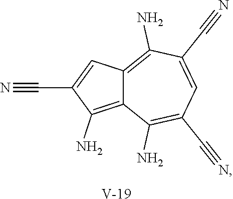

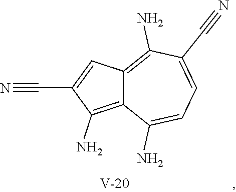

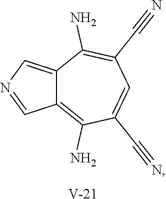

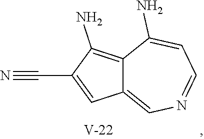

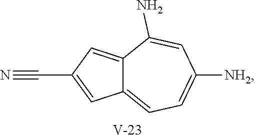

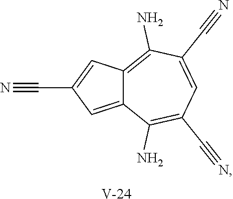

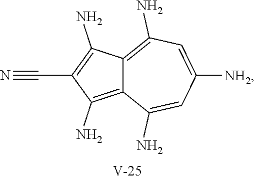

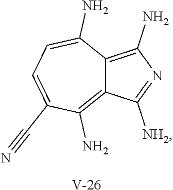

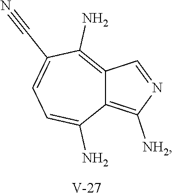

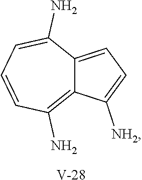

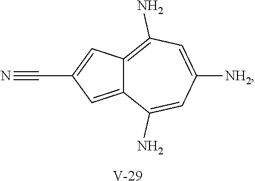

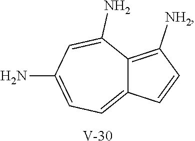

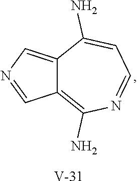

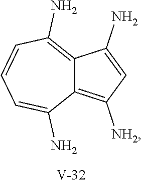

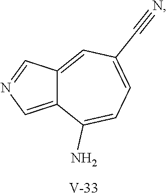

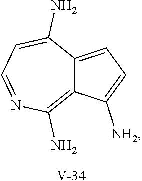

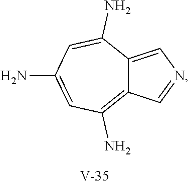

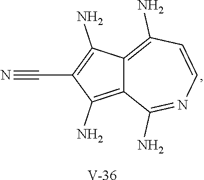

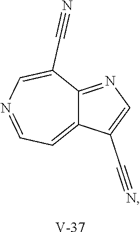

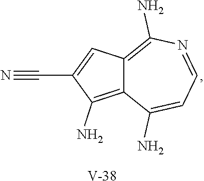

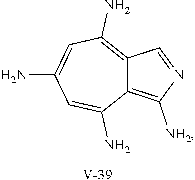

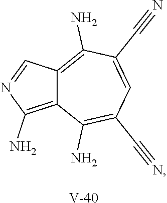

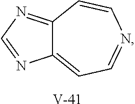

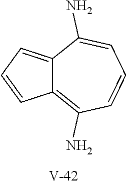

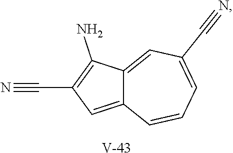

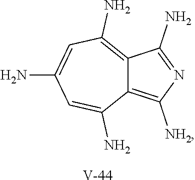

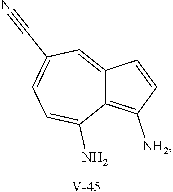

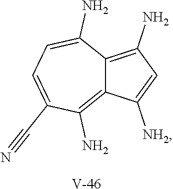

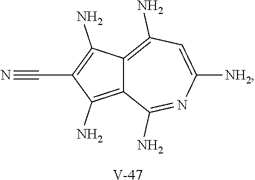

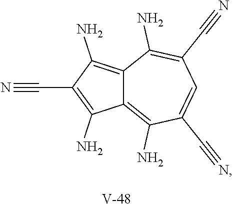

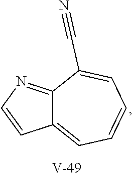

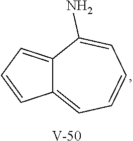

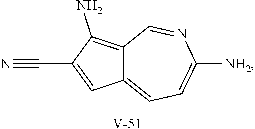

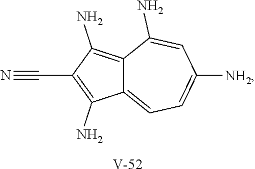

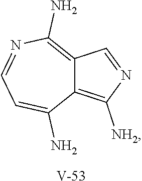

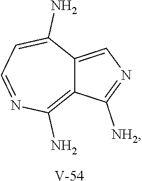

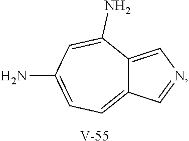

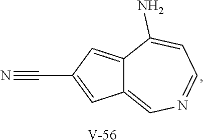

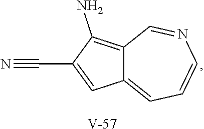

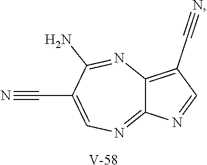

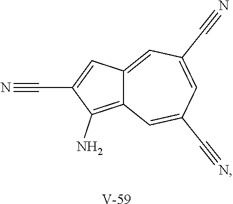

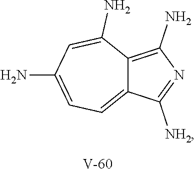

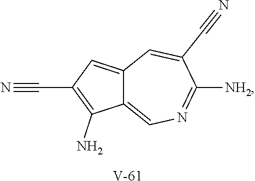

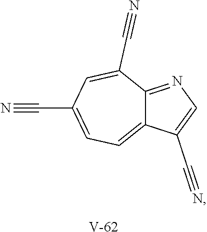

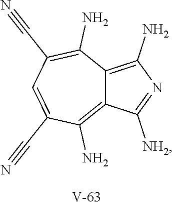

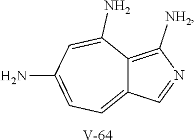

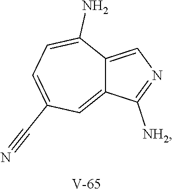

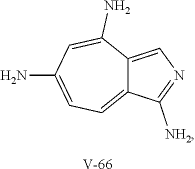

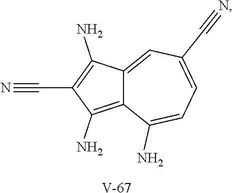

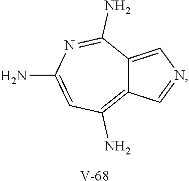

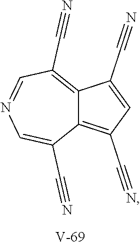

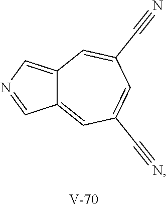

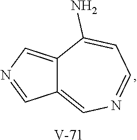

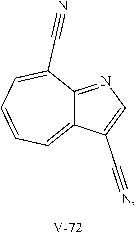

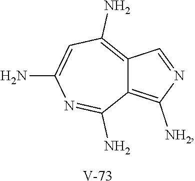

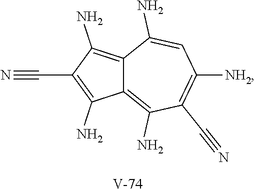

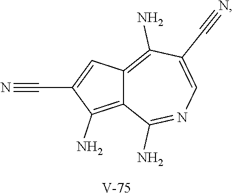

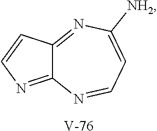

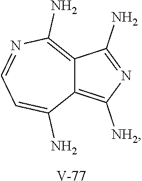

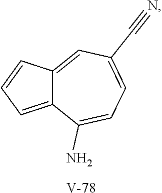

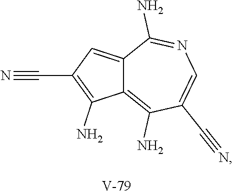

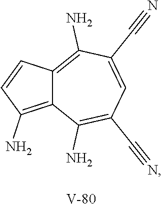

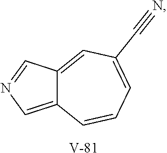

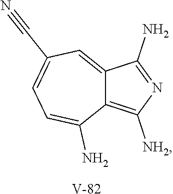

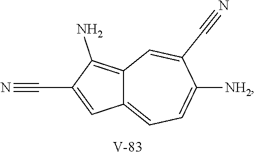

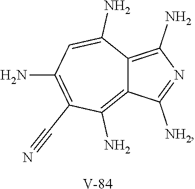

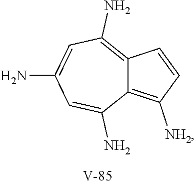

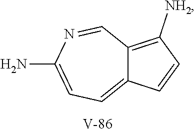

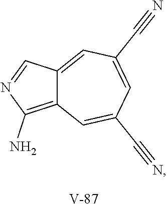

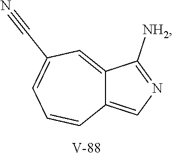

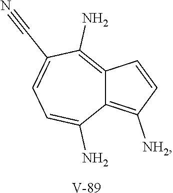

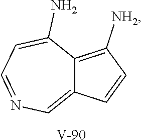

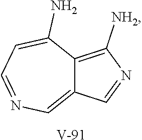

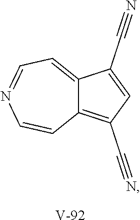

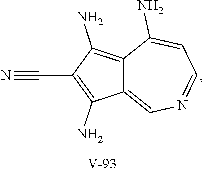

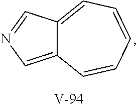

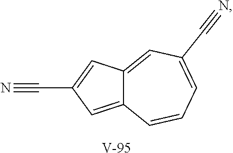

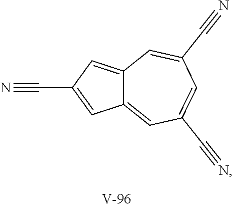

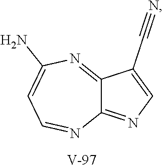

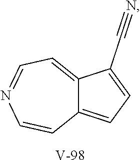

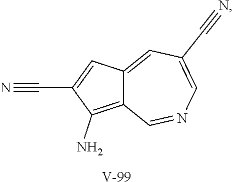

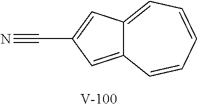 [SMILES: N#Cc1cc2cccccc-2c1]

In another aspect, the present application includes an organic light-emitting diode comprising at least one compound of the present application.

In another aspect, the present application includes a photocatalyst comprising at least one compound of the present application.

In another aspect, the present application includes a triplet quencher comprising at least one compound of the present application.

### III. Methods of Preparation

The compounds disclosed herein can be prepared by various synthetic processes. The choice of particular structural features and/or substituents may influence the selection of one process over another. The selection of a particular process to prepare a given compound is within the purview of the person of skill in the art. Some starting materials for are available from commercial chemical sources. Other starting materials, are readily prepared from available precursors using straightforward transformations that are well known in the art.

In some embodiments, the compounds of Formula V generally can be prepared according to the processes illustrated in the Schemes below. In the structural formulae shown below the variables are as defined in Formula V unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

Accordingly, in some embodiments, the compounds of the present application can be prepared as shown in the retrosynthetic Schemes below. The term “Hal” as used in the Schemes refers to halogen. For example, it can refer to Br, Cl, or I.

In some embodiments, the compound V-1 of Formula V is prepared as shown in Scheme A. Therefore, the aldehyde A-1 can be protected through acetal formation to generate compound A-2. Nucleophilic aromatic substation or carbon-nitrogen cross coupling of the halides can provide the compound A-3. Upon deprotection to reveal aldehyde A-4, electrophilic aromatic halogenation can be used to provide compound A-5. A-5 can undergo an imine formation followed by an aldol reaction to generate compound A-7. Deprotection (if necessary) of the anilinic nitrogens of compound A-7 provides A-8, which can react with benzaldehyde to provide A-9. Activation of the carboxylic acid moiety affords compound A-10, which can react with diazomethane to form compound A-11. Cyclisation reaction of A-11 with simultaneous loss of N₂ can provide bicyclic structure A-12. Halogenation at the alpha position of the ketone can afford compound A-13. Dehydration of the ketone group with simultaneous imine deprotection can provide the azulene structure A-14. Substitution of the halogens with nitrile groups can generate the compound V-1.

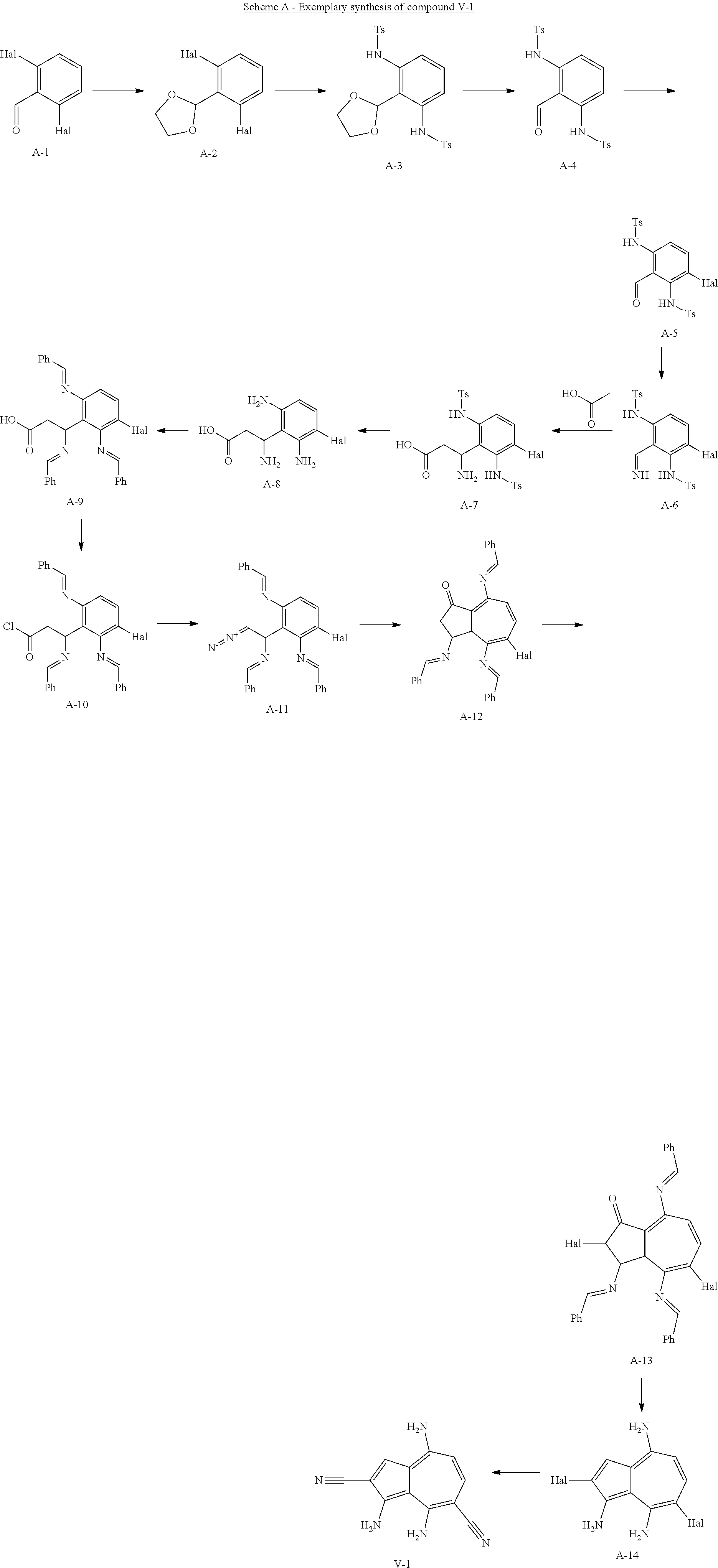

In some embodiments, the compound V-2 is prepared as shown in Scheme B. Therefore, the pentenone B-1 can be dihalogenated to provide the dihalide B-2. Elimination of B-2 can afford the vinyl halide B-3. 1,2-reduction of B-3 can lead to the alcohol B-4, which can be transformed through a substitution reaction to provide dihalide B-5. B-5 can be converted into the dimethylacetal B-6. Cyclisation of B-6 forms the cyclopropene B-7. After deprotection of the dimethylacetal moiety, cyclopronenone B-8 can react with TMS-propynylene to form the cyclopentadienone B-9. Removal of the TMS group and oxidation of the resulting B-10 can afford the trimethylester B-11. The three methyl ester groups can be converted to nitrile groups and the ketone to imine with ammonia to obtain B-12. Protection of the imine can provide B-13 followed by a cyclisation reaction with ethanimine can afford the azulene-type structure B-14. Deprotection of B-14 provides V-2.

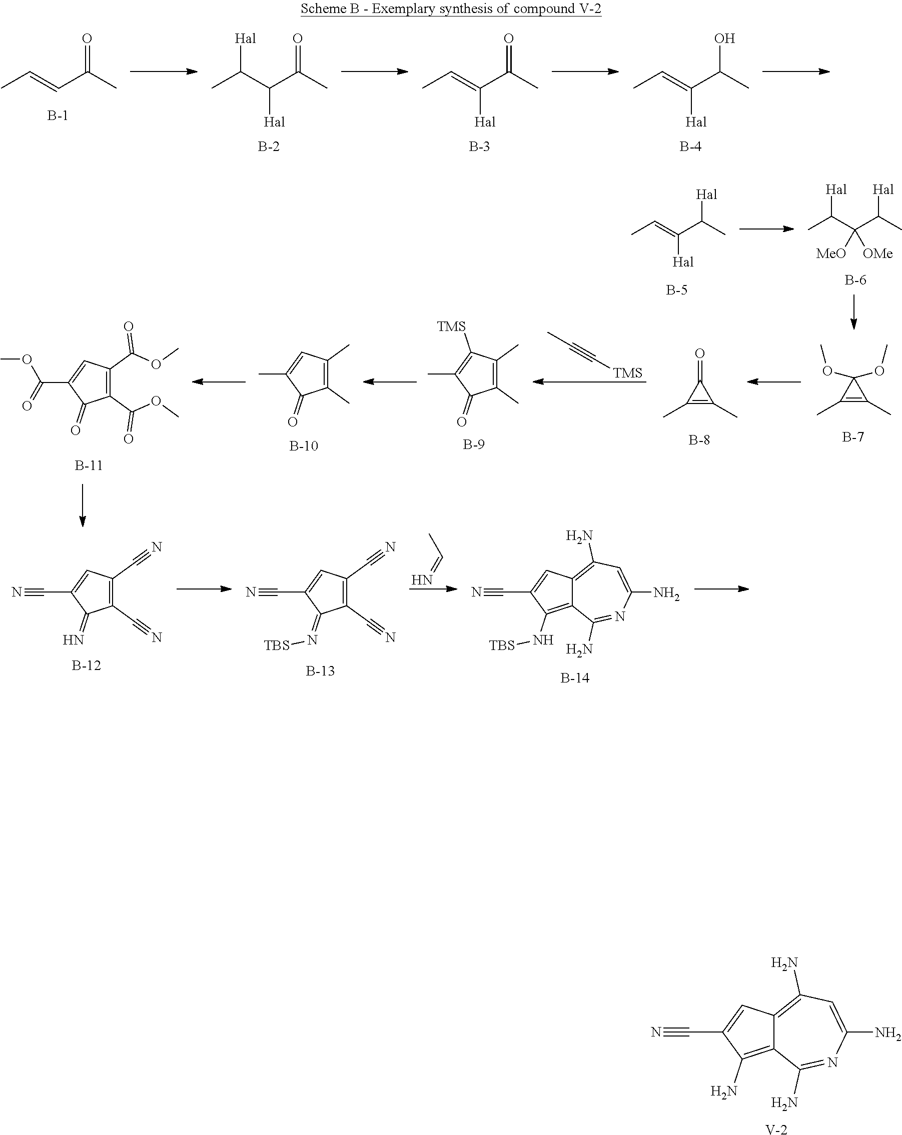

In some embodiments, the compound V-5 is prepared as shown in Scheme C. Therefore, dimethyl imidazole C-1 can be monoprotected at one nitrogen to provide C-2. The methyl groups can then be halogenated to obtain C-3. Nucleophilic substitution of C-3 can afford C-4. Deprotonation of C-4 followed by nucleophilic acyl substitution with for example DMF can provide dialdehyde C-5. Deprotection of the imidazolyl nitrogen (C-6) and cyclisation with ammonia can lead to V-5.

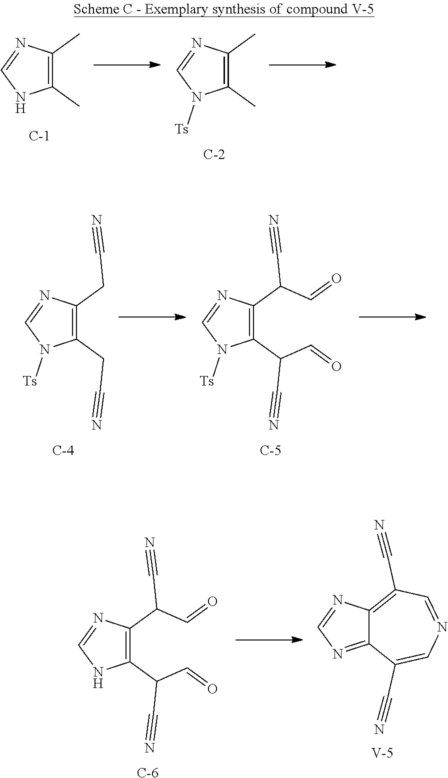

In some embodiments, the compound V-6 is prepared as shown in Scheme D. Therefore, oxalic acid (D-1) can be converted to oxamidine (D-2), which cyclises with formic acid to provide compound D-3. Separately, malonic acid D-4 can be halogenated at the alpha position to afford D-5. Nucleophilic substitution of the halide with nitrile can form D-6. The dicarboxylic acid groups can be converted into malonamidine compound D-7. D-3 can be reacted with D-7 in a condensation reaction to afford V-6.

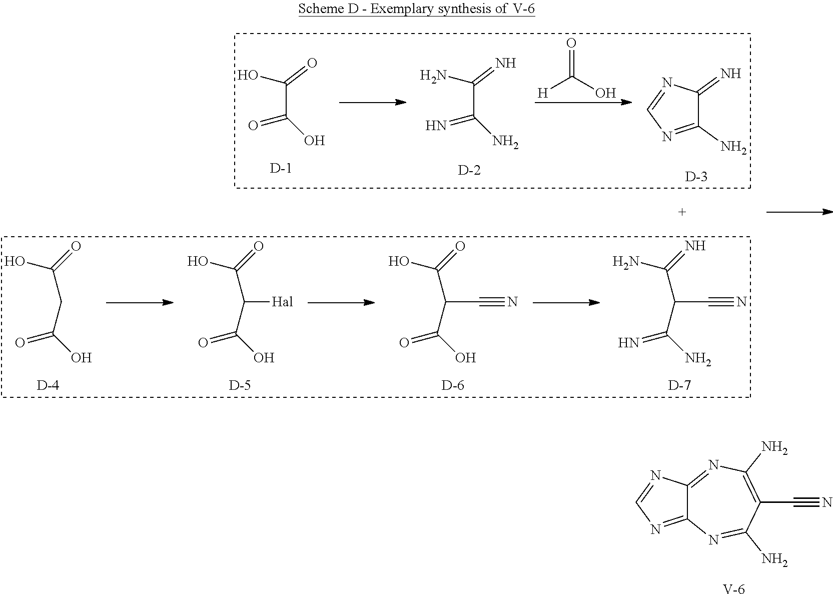

In some embodiments, the compound V-7 is prepared as shown in Scheme E. Therefore, pyrrole dicarboxylic acid E-1 can be transformed into dinitrile E-2 using for example KSCN. The pyrrole nitrogen in E-2 can be protected with for example p-toluenesulfonyl chloride to form E-3. One of the identical nitriles can be alkylated with for example methyl lithium and the resulting product captured with p-toluenesulfonyl chloride to yield E-4. Separately, 3-hydroxypropionitrile can be oxidized partially to afford cyanoacetaldehyde. E-4 can react with cyanoacetaldehyde under dehydration to form E-5. E-5 can be cyclized under basic conditions to form E-6. The two p-toluenesulfonyl groups in E-6 can be deprotected to afford V-7.

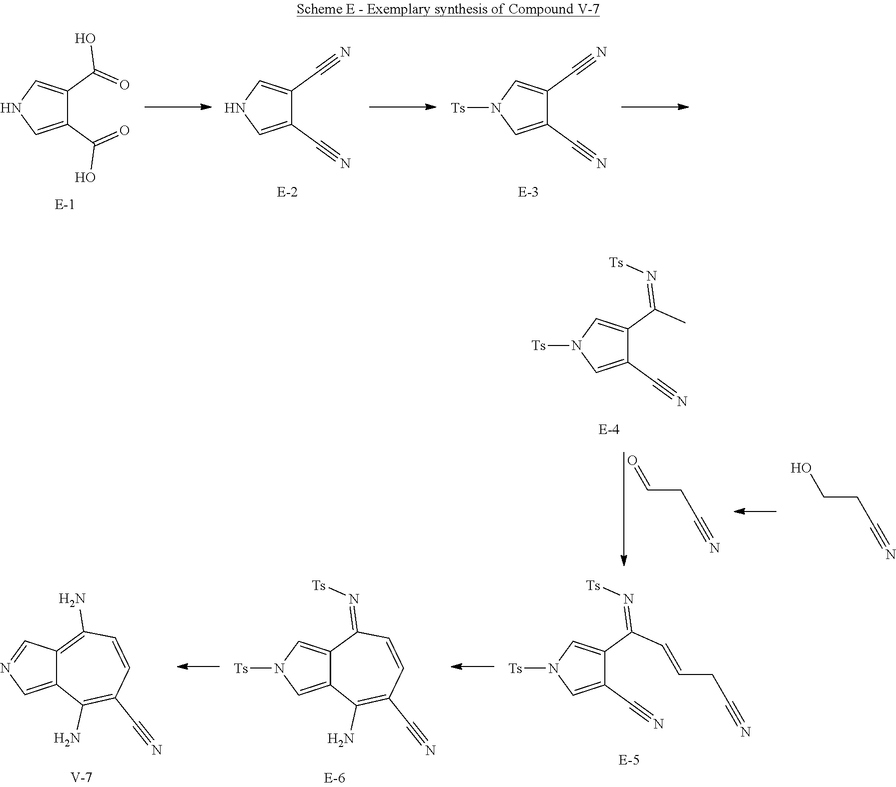

In some embodiments, the compound V-104 is prepared as shown in Scheme F. Therefore, differential protection of the hydroxypyrrole F-1 can provide F-2. Dihalogentation at the 2 and 4 positions of the pyrrole can afford F-3. Separately, p-aminophenol F-4 can be protected to give F-5. Transition metal-catalyzed direct borylation can provide the boronic ester F-6. Transition metal-catalysed coupling reaction of the halide F-3 and the boronic ester F-6 can afford F-10. Separately, pyrrole dicarboxylic acid F-7 can be esterified to give F-8, which can undergo a cyclisation with formamidine to give the azulene-type ring structure F-9. F-9 can be coupled to two equivalents of F-10 through a nucleophilic aromatic substitution, or a transition-metal catalyzed cross-coupling to provide F-11. Global deprotection can afford compound V-104.

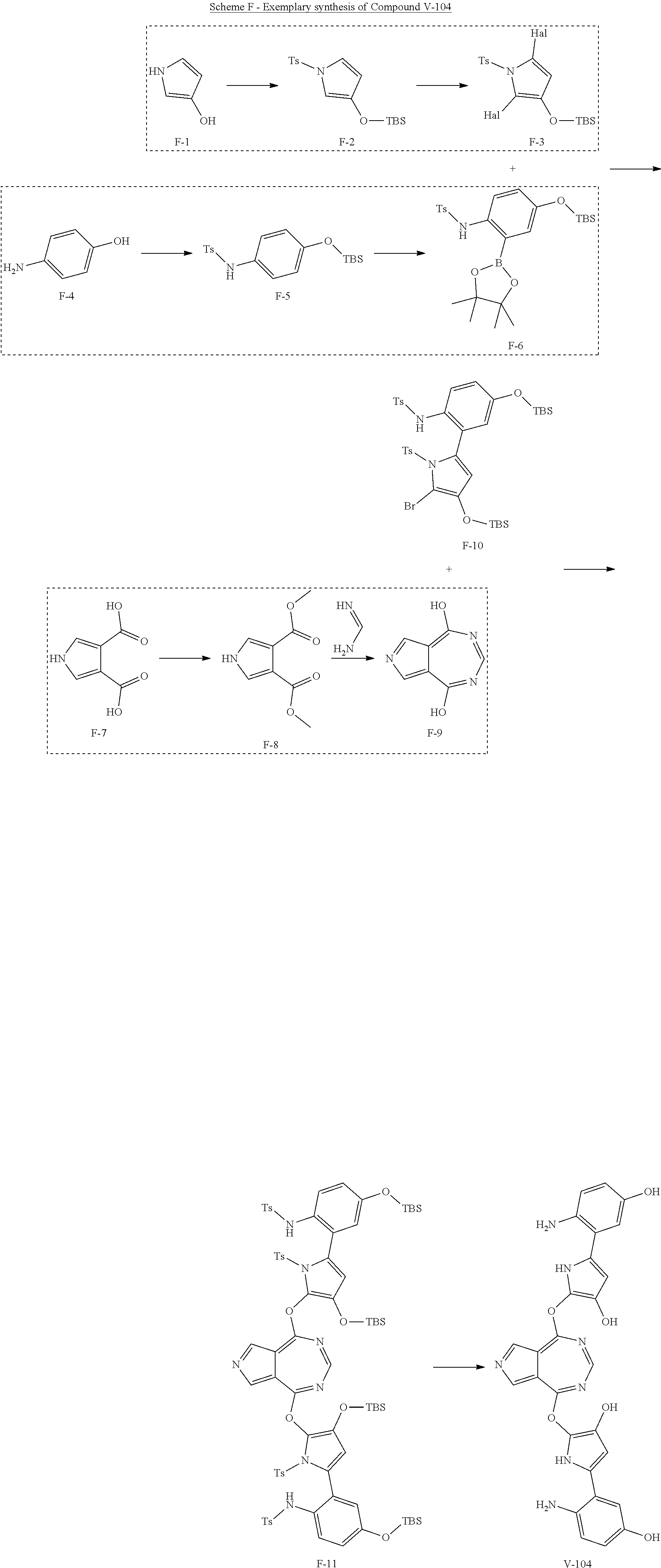

In some embodiments, the compound V-112 is prepared as shown in Scheme G. Therefore, triisopropylsilyl-protected glycolaldehyde can be alkylated with trifluoromethanesulfonyl-functionalized glycolaldehyde to give G-1. Treating G-1 with ammonia can afford G-2. Double formylation of G-2 with dimethylformamide can provide G-3. Separately, phenanthrene-2-carboxylic acid can be reacted with ammonia to afford amidine G-5. G-3 and G-5 can be condensed to yield the azulene derivative G-6. Subsequently, deprotection of the triisopropylsilyl-protected alcohol in G-6 can afford G-7. This free alcohol in G-7 can be functionalized with trifluoromethanesulfonyl chloride to provide G-8. Transition-metal catalyzed cross-coupling of G-8 with phenanthrene-2-boronic acid can afford compound V-112.

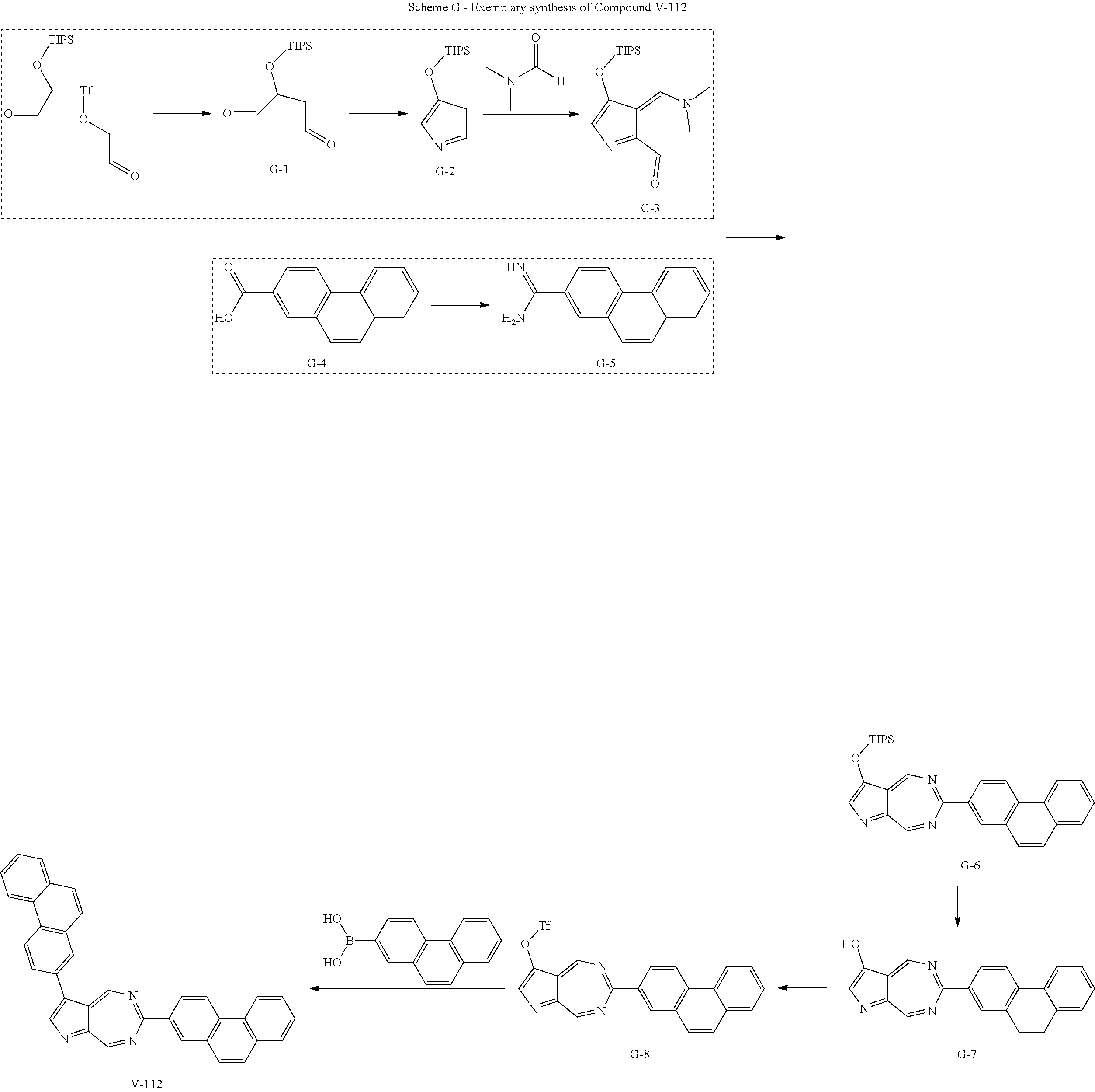

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

### IV. Methods and Uses of the Application

In another aspect, the present application also includes a use of a compound disclosed in an organic light-emitting diode.

In another aspect, the compound is used as an emitter or a dopant.

In another aspect, the present application also includes a method of preparing an organic light-emitting diode comprising providing at least one compound disclosed herein as an emitter or a dopant.

In another aspect, the present application also includes an organic-light emitting diode comprising at least one compound disclosed herein.

In another aspect, the present application includes a use of a compound a disclosed herein as a photocatalyst.

In another aspect, the present application includes a method of performing photocatalysis comprising contacting at least one compound disclosed herein with a mixture requiring a photocatalyst and performing a photocatalytic transformation on the mixture.

In another aspect, the present application includes a use of a compound disclosed herein in the generation of organic laser.

In another aspect, the present application includes a method of generating organic laser comprising providing at least one compound disclosed herein as a light emitter.

In another aspect, the present application also includes an organic-laser comprising at least one compound disclosed herein.

In another aspect, the present application includes a use of a compound disclosed herein in the enhancement of photostability.

In some embodiments, the compound is used as a triplet quencher.

In some embodiments, the present application includes a method of enhancing photostability comprising providing at least one compound disclosed herein as a triplet quencher.

In some embodiments, the at least one compound disclosed herein is selected from compounds of Formulae III, V, VII, IX, and XII, or a salt and/or tautomer thereof. In some embodiments, the at least one compound disclosed herein is selected from compounds of

Formula V, or a salt and/or tautomer thereof.

### EXAMPLES

The following non-limiting examples are illustrative of the present application.

### Example 1 Computational Details

Computations were performed on the Niagara supercomputer at the SciNet HPC Consortium^{23,24} and on the Cedar supercomputer situated at the Simon Fraser University in Burnaby. Ground state conformational ensembles were generated using crest²⁵ (version 2.10.1) with the iMTD-GC^{26,27} workflow (default option) using the GFN2-xTB^{28,29}//GFN-FF³⁰⁻³² composite method. The lowest energy conformer was first reoptimized using xtb³³ (version 6.3.0) at the GFN2-xTB^{28,29} level of theory, followed by another reoptimization using Orca^{34,35} (version 4.2.1) at the B97-3c³⁶ level of theory. The corresponding geometries were used for subsequent ground- and excited state calculations. Excited state single points at the RKS-ωB2PLYP′³⁷/def2-SVP³⁸ and the UKS-ωB2PLYP³⁷/def2-SVP³⁸ levels of theory were performed using Orca^{34,35} (version 4.2.1). Single points at the SF-PBE50³⁹⁻⁴⁴/def2-SVP⁴⁵ level of theory, and ground- and excited state geometry optimizations at the B3LYP⁴⁶⁻⁴⁸/6-31G*⁴⁹⁻⁵¹ level of theory were performed using Q-Chem⁵² (versions 5.2, 5.3 and 5.4, respectively). Single points at the RI-ADC(2)⁵³⁻⁶⁶/cc-pVDZ,⁴⁵ RI—SOS-ADC(2)⁵³⁻⁶⁸/cc-pVDZ,⁴⁵ RI—CC2^{61,63,65,66}/cc-pVDZ,⁴⁵ RI—SOS—CC2^{63,65,66,68,69}/cc-pVDZ,⁴⁵ RI—CIS(D)^{63,66,70,71}/cc-pVDZ,⁴⁵ RI—SCS—CIS(D)^{61,63,66-68,70-72}/cc-pVDZ⁴⁵ and RI—SOS—CIS(D)^{61,63,66-68,70-72}/cc-pVDZ⁴⁵ levels of theory were performed using MRCC^{73,74} (version 2020). Fractional occupation number weighted electron density⁷⁵ (FOD) single point calculations were performed using Orca^{34,35} (version 4.2.1). Importantly, in the Orca version used (version 4.2.1), the perturbative doubles correction is not applied to the excited triplet states when using restricted Kohn-Sham (RKS) calculations.⁷⁶ Hence, to indicate this explicitly in our results, the corresponding method ωB2PLYP′ was termed as opposed to ωB2PLYP, which was obtained using unrestricted Kohn-Sham (UKS) calculations. For all excited state calculations, unless noted otherwise, four roots were chosen each for both the singlet and the triplet manifold.

After each geometry optimization, it was checked systematically whether the structure obtained afterwards still has the same connectivity as the input molecular graph. The xyz2 mol package⁷⁷ was used to convert the optimized Cartesian coordinates to the output SMILES string. Subsequently, all multi bonds in both the input SMILES string and the output SMILES string were converted to single bonds and RDKit⁷⁸ was used to canonicalize the resulting SMILES. This allows direct comparison of the molecular connectivity by comparing the resulting strings and ignores potential changes of multi bonds in extended π-systems. However, it is very robust for detecting skeleton rearrangements. All properties of structures with changed connectivities based on that procedure were disregarded in our analysis. Additionally, the planarity of the core atoms in the optimized ground-state geometries was quantified by fitting a plane through the corresponding atoms that minimizes the mean distance of these atoms to that plane. The residual mean distance of all the atoms to the best fit plane is divided by the mean bond distance between the core atoms to provide the deviation from planarity as a dimensionless quantity. Planarity is then defined as one minus the deviation from planarity.

### Example 2 Results

Bottom-up Rules. Inspired by the structures of the molecules known to violate Hund's first rule,^{4,16,79-81} both in the ground state and in the first excited states, and the premise to maximize dynamic spin polarization,⁸²⁻⁸⁴ it was realized that all of them comply with the following construction rules, which was termed the Ring-Bonds-Substitutions (RiBS) construction scheme:
1. Start with an alternant monocyclic hydrocarbon and identify the starred and unstarred positions.
2. Optionally, connect pairs of starred or unstarred carbon atoms, respectively, with new bonds forming non-alternant annulated multicyclic systems but retain the starred and unstarred labels from the parent monocyclic hydrocarbon.
3. Substitute a subset of either only starred or only unstarred methine groups with donor groups, and a subset of the respective other set of methine groups with acceptor groups.

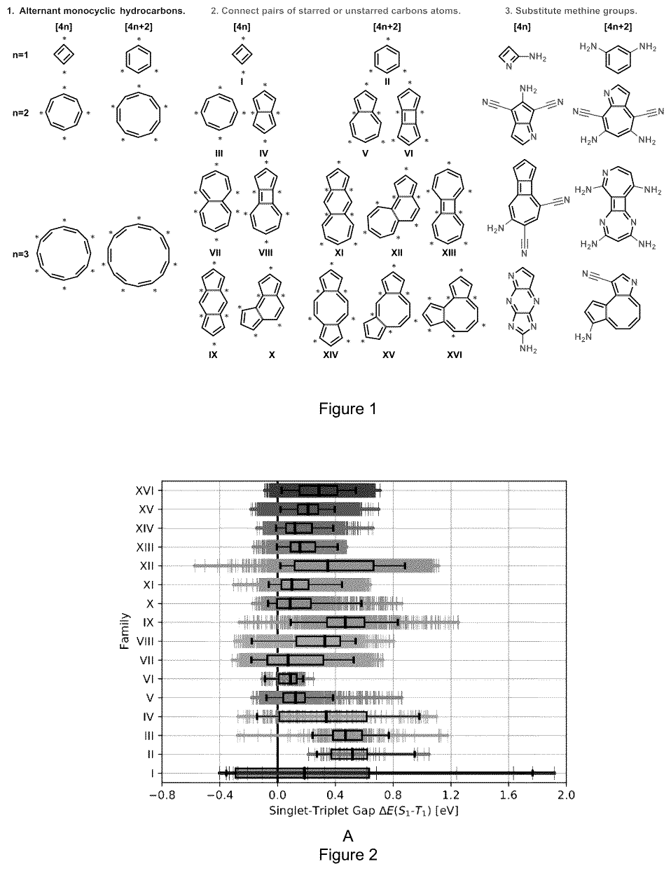

Next, it was decided to use these rules to build a systematic library of INVEST candidates. To keep the number of compounds tractable, it was decided to investigate systems with up to 14 atoms in the parent monocyclic hydrocarbon, i.e., limited to [4n] and [4n+2] π-systems with n going from 1 to 3. Furthermore, only ring sizes from 4 to 8 were allowed in the final structures to avoid both high ring strain and extensive double bond stereoisomerism in the resulting structures, and the total number of annulated rings was restricted to be at most 3. Finally, the allowed substitutions were restricted to replacement of methine (C—H) with either nitrogen (N), aminomethine (C—NH₂) and cyanomethine (C—CN). These substitutions were chosen to either introduce high donor or high acceptor ability, and to keep the total number of added atoms minimal. All permutations of these substitutions leading to unique compounds were allowed. The construction of the candidate structures is illustrated in FIG. 1. A total of 69,201 unique molecules was generated that way, belonging to 16 distinct families of compounds, denoted henceforth I-XVI (FIG. 1).

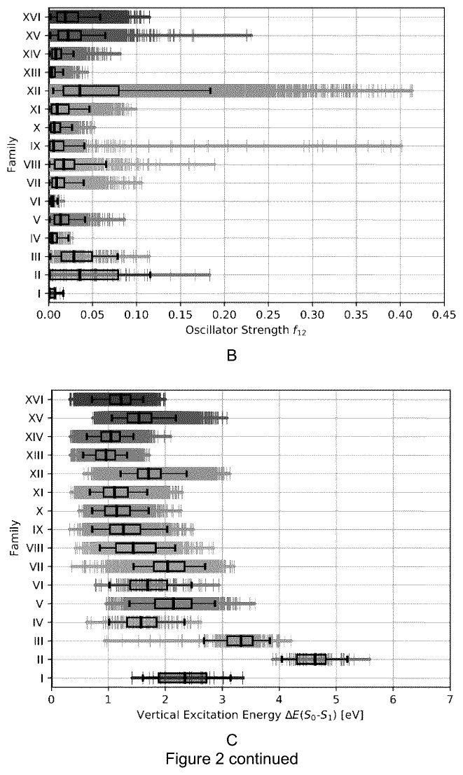

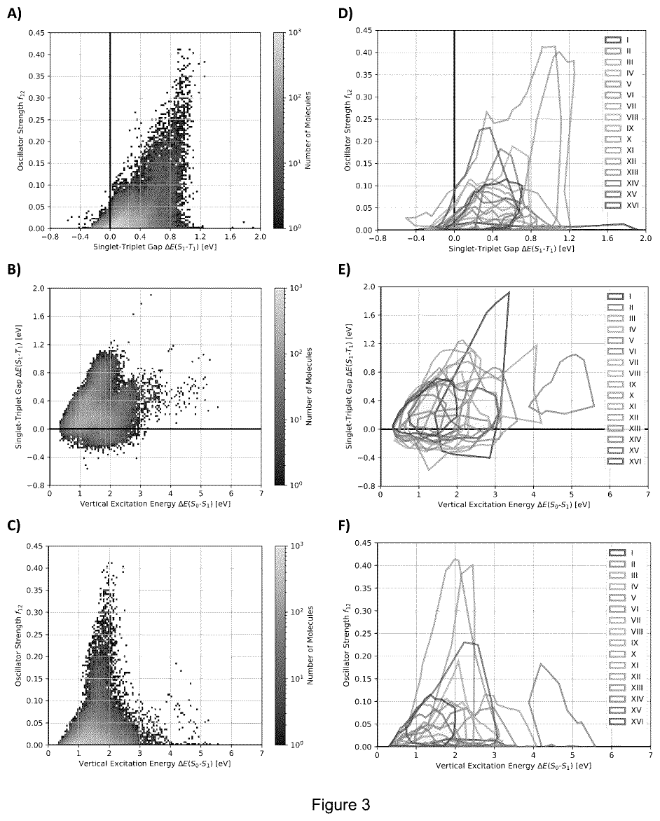

Virtual Screening. After mapping the structural space to be investigated, high-throughput virtual screening of the corresponding excited state properties was performed. The computational workflow established previously to increase throughput without sacrificing rigor was refined (cf. Example 1 Computational Details).¹⁹ Overall, this workflow consisted of conformer sampling using both a force field and semiempirical quantum chemistry followed by geometry optimization using a density functional approximation and excited state single point evaluations with both time-dependent density functional approximations and correlated excited state methods. Only 19 molecules changed their geometry during either the conformer sampling or the geometry optimization and, hence, were excluded from any further property evaluation as they correspond to unstable structures. Out of all the computational methods applied to simulate the excited states, ADC(2)/cc-pVDZ was selected as it has been shown to be robust in previous work on INVEST emitters.¹⁹ The property ranges of the entire set of compounds at the ADC(2) level of theory, divided into the 16 compound families, are depicted in FIG. 2. Additionally, the corresponding histograms over all combinations of two properties are illustrated in FIG. 3. Results at various alternative levels of theory are provided in Example 4.

First, it was found that the STG histogram has a peak just slightly above 0 indicating that the bottom-up rules indeed tend to create molecules with low STGs. Importantly, 7643 of the structures, i.e., 11% of all the structures generated, are predicted to have an inverted STG. Furthermore, every family, except family II, has at least one INVEST candidate. Of all the families, family I has with 43% the highest fraction of INVEST candidates, followed by VII with 38%. However, in absolute numbers, family VII has with 3260 INVEST candidates much more as it allows for a much larger variety of substituted compounds. Additionally, as observed previously,⁸⁵ a clear trade-off between low STG and high oscillator strength (OS) is visible. The highest OS observed for an INVEST candidate amounts to 0.084 and belongs to family XII. When looking for INVEST candidates with predicted OS larger than 0.01 and potential as organic emitters, it was found that most families have at least a few. Very promising in that regard are heptalenes with 13% and azulenes with 5% of all the molecules having both inverted STGs and appreciable OS. Notably, there is no obvious trade-off between any of the other properties. It was also found that the vertical excitation energies of the INVEST candidates cover the entire range of visible light of 1.7 eV to 3.3 eV, with a significantly lower fraction of molecules close to the high energy end. It was observed that the molecules with higher vertical excitation energies tend to have lower OS. Important family-specific properties are summarized in Table 1.

TABLE 1
Molecular families considered and summary of virtual screening results at the
ADC(2)/cc-pVDZ level of theory. INVEST candidates refers to compounds with predicted
negative singlet-triplet gaps. INVEST emitters refers to INVEST candidates with predicted
oscillator strength larger than 0.01.

| | | Parent | | | |
|---|---|---|---|---|---|
| | | Ring | | INVEST | INVEST |
| Family | Parent Compound Name | Size | Compounds | Candidates | Emitters |
| I | Cyclobuta-1,3-diene | 4 | 21 | 9 (43%) | 0 (0%) |
| II | Benzene | 6 | 56 | 0 (0%) | 0 (0%) |
| III | Cycloocta-1,3,5,7-tetraene | 8 | 336 | 5 (1%) | 0 (0%) |
| IV | Pentalene⁸⁶ | 8 | 248 | 59 (24%) | 1 (0%) |
| V | Azulene⁸⁶ | 10 | 1493 | 272 (18%) | 76 (5%) |
| VI | Bowtiene⁸⁷ | 10 | 126 | 28 (22%) | 0 (0%) |
| VII | Heptalene⁸⁶ | 12 | 8513 | 3260 (38%) | 1131 (13%) |
| VIII | Zurlene⁸⁸ | 12 | 2591 | 421 (16%) | 108 (4%) |
| IX | s-Indacene⁸⁹ | 12 | 1784 | 38 (2%) | 0 (0%) |
| X | as-Indacene⁹⁰ | 12 | 1296 | 349 (27%) | 29 (2%) |
| XI | Anthrazulene⁹¹ | 14 | 10709 | 1686 (16%) | 136 (1%) |
| XII | Phenazulene⁹² | 14 | 15551 | 444 (3%) | 242 (2%) |
| XIII | Dicyclohepta[a,c]cyclobutene⁹³ | 14 | 3996 | 207 (5%) | 1 (0%) |
| XIV | Dicyclopenta[a,e]cyclooctene⁹⁴ | 14 | 3996 | 265 (7%) | 14 (0%) |
| XV | Dicyclopenta[a,d]cyclooctene⁹⁴ | 14 | 10709 | 400 (4%) | 182 (2%) |
| XVI | Dicyclopenta[a,c]cyclooctene⁹⁴ | 14 | 7776 | 200 (3%) | 8 (0%) |
| I-XVI | — | — | 69201 | 7643 (11%) | 1928 (3%) |

Next, the structures of some of the best-performing INVEST candidates of each family were investigated. The molecules with the lowest STGs in each family are listed in Table 2 together with their predicted properties at the ADC(2) level of theory. Interestingly, nitrogen substitutions in the core structure seem to be more abundant in these molecules than introducing amino or cyano groups. Furthermore, it seems that for most of the families the suitable core substitutions either only donate electron density to the core (amino groups) or only withdraw electron density from the core (cyano groups, nitrogen atoms). However, the substitution patterns leading to the lowest STGs differ considerably between the 16 families investigated.

Additionally, the structures of molecules with inverted STG that have the highest OS in each family were studied, and they are provided in Table 3 with their simulated excited state properties. Notably, as there was no benzene derivative with an inverted STG in the compound space investigated, a structure was selected with a comparably low STG within that family (i.e., STG<0.35 eV) and a very high OS instead. Compared to the structures with the lowest STGs, nitrogen substitutions seem to be present at a similar rate. Moreover, it seems that substitution patterns that introduce two electronically distinct sites in the core structures are suitable. Nevertheless, it was observed again that substitution patterns are very different between the families investigated making it complicated to derive additional design principles by visual inspection of the molecular graphs.

TABLE 2
Molecules with lowest predicted singlet-triplet gap at the ADC(2)/cc-pVDZ level of
theory in each of the 16 families studied.

| | | ΔE | | ΔE |
|---|---|---|---|---|
| | | (S₁-T₁) | | (S₀-S₁) |
| Family | Molecule | [eV] | f₁₂ | [eV] |
| I | 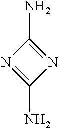 | −0.40 | 0.000 | 2.86 |
| | 1 | | | |
| II | 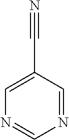 | 0.21 | 0.000 | 4.21 |
| | 2 | | | |
| III | 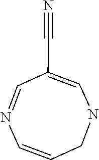 | −0.28 | 0.000 | 1.24 |
| | 3 | | | |
| IV | 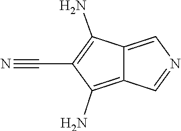 [SMILES: N#CC1=C(N)C2=CN=CC2=C1N] | −0.27 | 0.000 | 1.91 |
| | 4 | | | |
| V | 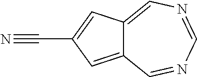 [SMILES: N#Cc1cc2cncncc-2c1] | −0.18 | 0.001 | 2.42 |
| | 5 | | | |
| VI | 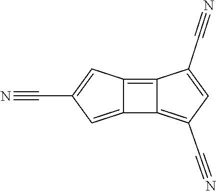 [SMILES: N#Cc1cc2c3c(C#N)cc(C#N)c-3c-2c1] | −0.11 | 0.003 | 1.71 |
| | 6 | | | |
| VII | 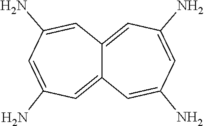 [SMILES: NC1=CC(N)=CC2=CC(N)=CC(N)=CC2=C1] | −0.32 | 0.000 | 1.17 |
| | 7 | | | |
| VIII | 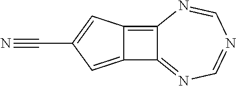 [SMILES: N#CC1=CC2=C3N=CN=CN=C3C2=C1] | −0.30 | 0.000 | 1.59 |
| | 8 | | | |
| IX | 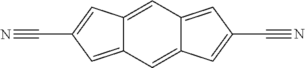 [SMILES: N#CC1=Cc2cc3c(cc2=C1)C=C(C#N)C=3] | −0.27 | 0.000 | 0.76 |
| | 9 | | | |
| X | 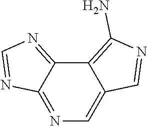 [SMILES: NC1=c2c(cnc3c2=NC=N3)C=N1] | −0.17 | 0.003 | 1.31 |
| | 10 | | | |
| XI | 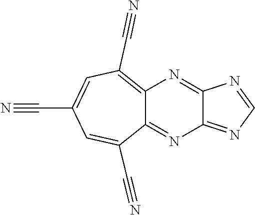 [SMILES: N#Cc1cc(C#N)c2nc3ncnc3nc2c(C#N)c1] | −0.31 | 0.000 | 1.21 |
| | 11 | | | |
| XII | 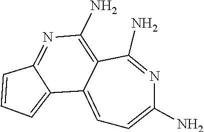 [SMILES: Nc1ccc2c(c(N)n1)c(N)nc1cccc12] | −0.57 | 0.021 | 1.26 |
| | 12 | | | |
| XIII | 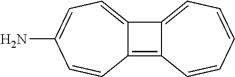 [SMILES: Nc1ccc2c3cccccc-3c-2cc1] | −0.17 | 0.000 | 0.65 |
| | 13 | | | |
| XIV | 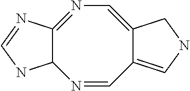 [SMILES: C1=N/C2=N/C=C3/CNC=C3/C=N\C2N1] | −0.14 | 0.003 | 1.11 |
| | 14 | | | |
| XV | 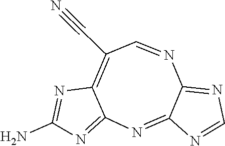 [SMILES: N#Cc1cnc2ncnc-2nc2nc(N)nc1-2] | −0.18 | 0.008 | 1.48 |
| | 15 | | | |
| XVI | 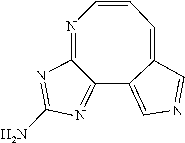 [SMILES: Nc1nc2ncccc3cncc-3c-2n1] | −0.09 | 0.002 | 1.35 |
| | 16 | | | |

TABLE 3
Molecules predicted to possess inverted singlet-triplet gaps with the highest predicted
oscillator strength at the ADC(2)/cc-pVDZ level of theory in each of the 16 families studied. For
family II, as there was no molecule with a predicted inverted singlet-triplet gap, the compound
with highest oscillator strength and a singlet-triplet gap below 0.35 eV was selected instead.

| | | ΔE(S₁-T₁) | | ΔE(S₀-S₁) |
|---|---|---|---|---|
| Family | Molecule | [eV] | f₁₂ | [eV] |
| I | 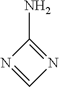 | −0.31 | 0.001 | 1.60 |
| | 17 | | | |
| II | 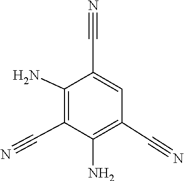 [SMILES: N#Cc1cc(C#N)c(N)c(C#N)c1N] | 0.34 | 0.116 | 3.94 |
| | 18 | | | |
| III | 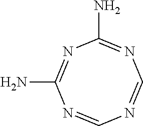 | −0.05 | 0.008 | 2.68 |
| | 19 | | | |
| IV | 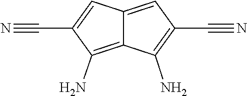 [SMILES: N#CC1=CC2=CC(C#N)=C(N)C2=C1N] | −0.06 | 0.023 | 1.33 |
| | 20 | | | |
| V | 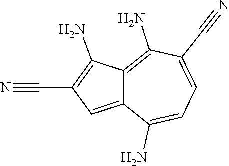 [SMILES: N#Cc1ccc(N)c2cc(C#N)c(N)c-2c1N] | −0.01 | 0.033 | 2.44 |
| | 21 | | | |
| VI | 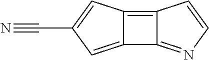 [SMILES: N#Cc1cc2c3ccnc-3c-2c1] | −0.08 | 0.006 | 2.05 |
| | 22 | | | |
| VII | 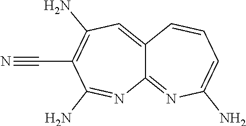 [SMILES: N#CC1=C(N)C=C2C=CC=C(N)N=C2N=C1N] | −0.01 | 0.054 | 1.87 |
| | 23 | | | |
| VIII | 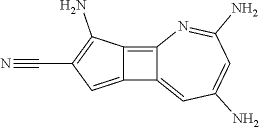 [SMILES: N#CC1=C(N)C2=C3N=C(N)C=C(N)C=C3C2=C1] | −0.01 | 0.044 | 2.03 |
| | 24 | | | |
| IX | 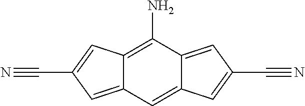 [SMILES: N#CC1=Cc2c(N)c3c(cc2=C1)C=C(C#N)C=3] | −0.17 | 0.008 | 1.18 |
| | 25 | | | |
| X | 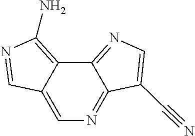 [SMILES: N#CC1=CN=c2c1ncc1c2=C(N)N=C1] | −0.03 | 0.020 | 1.00 |
| | 26 | | | |
| XI | 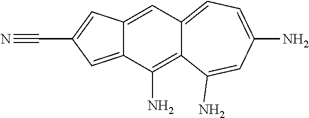 [SMILES: N#Cc1cc2cc3ccc(N)cc(N)c3c(N)c2c1] | −0.02 | 0.021 | 1.63 |
| | 27 | | | |
| XII | 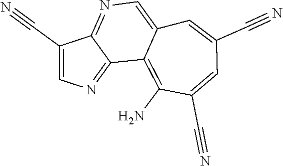 [SMILES: N#Cc1cc(C#N)c(N)c2c(cnc3c(C#N)cnc32)c1] | −0.07 | 0.084 | 2.04 |
| | 28 | | | |
| XIII | 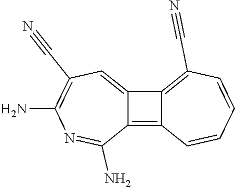 [SMILES: N#Cc1cc2c3c(C#N)ccccc-3c-2c(N)nc1N] | −0.01 | 0.010 | 1.18 |
| | 29 | | | |
| XIV | 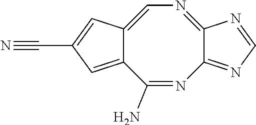 [SMILES: N#Cc1cc2cnc3ncnc-3nc(N)c-2c1] | −0.04 | 0.023 | 1.33 |
| | 30 | | | |
| XV | 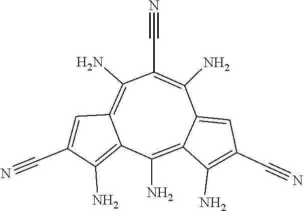 [SMILES: N#Cc1cc2c(N)c(C#N)c(N)c3cc(C#N)c(N)c-3c(N)c-2c1N] | −0.003 | 0.071 | 2.21 |
| | 31 | | | |
| XVI | 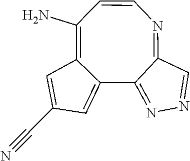 [SMILES: N#Cc1cc2c(N)ccnc3cnnc-3c-2c1] | −0.001 | 0.019 | 1.28 |
| | 32 | | | |

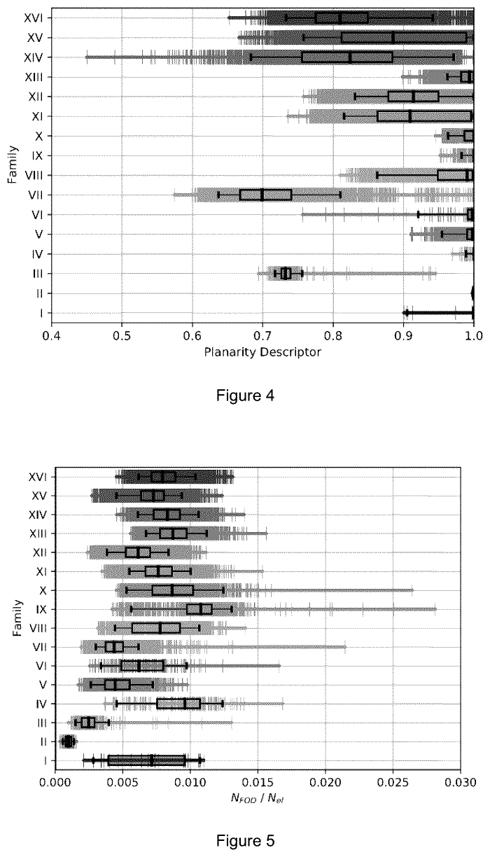

It was also tested whether simple structural descriptors could correlate with at least some of the properties of interest. It was observed that some of the structures deviate significantly from a planar core and that it also depends on the substitution pattern whether a given core will prefer to be planar or non-planar. Hence, the planarity of the atoms that constitute the cyclic core system was quantified via a newly introduced dimensionless descriptor that is based on fitting a plane to the corresponding atoms and using the average distance of these points to the best fit plane to quantify the deviation from planarity (cf. Computational Details). Importantly, a value of 1 corresponds to perfect planarity whereas smaller values indicate lower degrees of planarity. The corresponding descriptor ranges of the entire set of compounds divided into the 16 compound families, are depicted in FIG. 4. Most of the families showed a high degree of planarity with medians above 0.9. However, some of them showed significant deviations from planarity which align well with visually inspecting the corresponding structures. For instance, the vast majority of cycloocta-1,3,5,7-tetraenes (family III) have a planarity between 0.7 and 0.8 due to their well-known tub-shaped conformation preferred by the parent compound and most of the derivatives in the present dataset.^{95,96} The family with the lowest median planarity is heptalene which has been shown to prefer adopting a non-planar conformation with two boat-shaped 7-membered rings^{97,98} and the present simulations reproduce that well. However, the planarity of the families does not seem to be strongly related to the STGs or the other excited state properties of interest. There are families with a high deviation from planarity with many INVEST candidates such as heptalenes (family VII) or anthrazulenes (family XI) and families with almost perfect planarity with many INVEST candidates such as as-indacenes (family X) or pentalenes (family IV). This is important as all the organic INVEST molecules known previously were planar.

Validation. The next step was to validate the simulated properties by conducting a few computational controls. First, the importance of static electron correlation was investigated for describing the ground-state wavefunctions of the molecules under study by computing the integral of the fractional occupation number weighted electron density over all space normalized by the corresponding total number of electrons (N_{FOD}/Nₑₗ).⁷⁵ The ranges and distributions of this metric decomposed into the molecular families are illustrated in FIG. 5. These results show that most families have relatively low values with medians being below 0.010 for all compound classes except family IX, which has a median slightly above. This suggests that single reference excited state simulation methods are likely to provide reasonable results for most of the molecules studied. Nevertheless, all families showed significantly higher values than benzenes (family II), which are known to be well-described with single reference electronic structure methods. Very few compounds showed values above 0.020 where static correlation is starting to become somewhat relevant.⁷⁵ Importantly, when inspecting the correlation of this metric with the excited state properties of interest, a significant negative correlation was found with the vertical excitation energy of the first excited singlet (cf. Example 4 Supporting Information). Thus, the fractional electron occupation increased as the energy gap between the ground state and excited states decreased, which led to a higher probability for static correlation to be relevant.

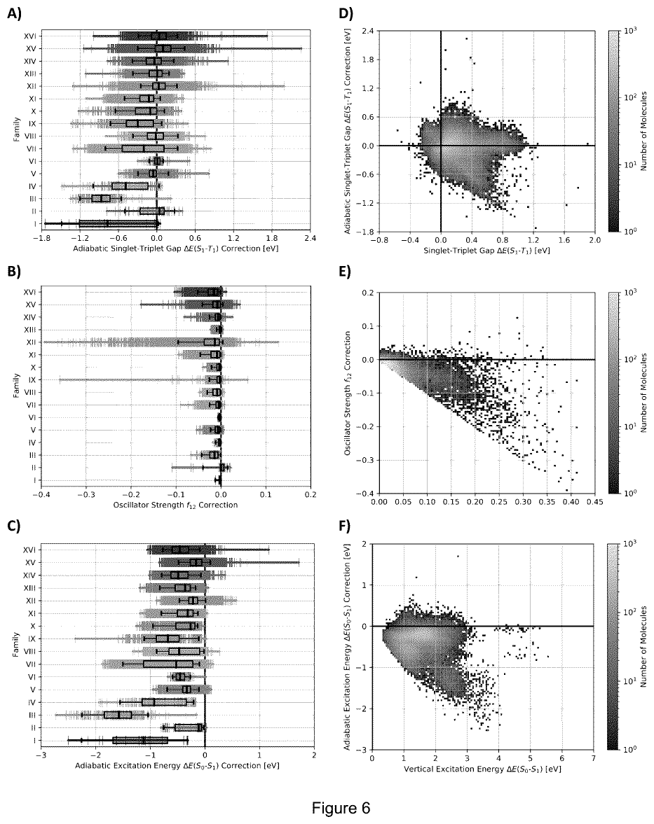

Additionally, the impact of excited state geometry optimization was quantified. To do that consistently on the large number of compounds studied without overstraining our computational resources, B3LYP/6-31G* was used as an efficient methodology to both reoptimize the ground-state geometry and optimize the first excited singlet and triplet geometries. Subsequently, single point calculations at the RKS-ωB2PLYP′/def2-SVP level of theory were performed at these three distinct geometries and the resulting excited state properties were calibrated against the ADC(2)/cc-pVDZ level using linear corrections (cf. Example 1 Computational Details and Example 4 Supporting Information). Overall, this approach estimated deviations in excited state properties due to geometry relaxation. The distributions of these property corrections for all the compounds studied in their respective class are illustrated in FIG. 6A-C. Additionally, the 2-dimensional histograms were also inspected with respect to both the respective property at the ADC(2) level of theory and the corresponding property correction caused by geometry relaxation (cf. FIG. 6D-F).

First, the results suggest that for most families the predicted adiabatic singlet-triplet gaps tend to be more negative than the vertical ones. In total, the number of INVEST candidates increases significantly from 7643 (11%) to 20228 (29%). A detailed account of the number of candidates in each of the structural families after correcting for geometry relaxation is in Example 4 Supporting Information. With respect to both the OS and the excitation energy between the ground state and the first excited singlet state, geometric relaxation tends to cause a decrease in the corresponding predicted properties. For the former, these corrections tend to be relatively small in magnitude. For the latter, corrections tend to be between 0 eV and −1 eV in most compound classes. However, for cyclobutadienes and cycloocta-1,3,5,7-tetraenes, the corrections tend to be larger in magnitude and primarily lie between −1 eV and −2 eV. Overall, these results suggest that geometry relaxation in the excited states of the compounds studied seems not to be a detriment to the inverted STGs.

### Example 3 Discussion

The study was set out by defining bottom-up rules for the construction of organic molecules with an increased probability to possess inverted STGs. Subsequently, these design rules were verified using high-throughput virtual screening by unveiling thousands of molecules from various structural families which were predicted to have an inverted STG. In the following discussion, the bottom-up design rules are rationalised, the computational findings examined, and the 16 molecular families investigated in detail to assess their potential for applications as organic electronic materials and as emissive materials in light-emitting diodes.

Bottom-up Rules. Previously, only a handful of structural motifs were known or predicted to violate Hund's first rule in the first excited singlet and triplet states, the most prominent structural family constituting azaphenalenes.^{7,8,13,14,19,99} Importantly, the violation of Hund's first rule has been discussed more extensively in the context of systems that can exist either as a ground state singlet or as a ground state triplet, as was the case for both cyclobuta-1,3-diene and cycloocta-1,3,5,7-tetraene.^{79,80} While not precisely the same question, it is still relevant in the context of violations of Hund's first rule in the first excited singlet and triplet states, i.e., INVEST compounds, as the underlying physical interactions leading to the corresponding violation are equivalent and it requires some alterations of the electronic structure to have one or the other situation. This means that violations of Hund's first rule in the electronic ground state can be intentionally promoted in the first excited states. Importantly, the common requirement to violate Hund's first rule is the presence of sufficient dynamic spin polarization in conjunction with small exchange integrals between the orbitals in question.^{4,16} Small exchange integrals are the consequence of small orbital overlap between the orbitals involved in the corresponding electronic transition. So, ideally, disjoint orbitals should be present.⁴ Maximizing dynamic spin polarization in the first excited singlet state can be achieved in compounds and states with singly occupied molecular orbital (SOMO) nodes being located at some of the nuclei that also possess fully occupied orbitals which are relatively high in energy and are easily polarized.⁸⁴ Both these requirements are satisfied in conjugated π-systems where the SOMOs have orbital nodes at every other atom and some of the fully occupied orbitals that are closest in energy to the SOMOs have significant orbital coefficients at the positions of the nodes of the SOMOs.⁸⁴ Notably, the SOMOs of the first excited state referred to above typically correspond to the HOMO and LUMO in the ground state of the corresponding compound.

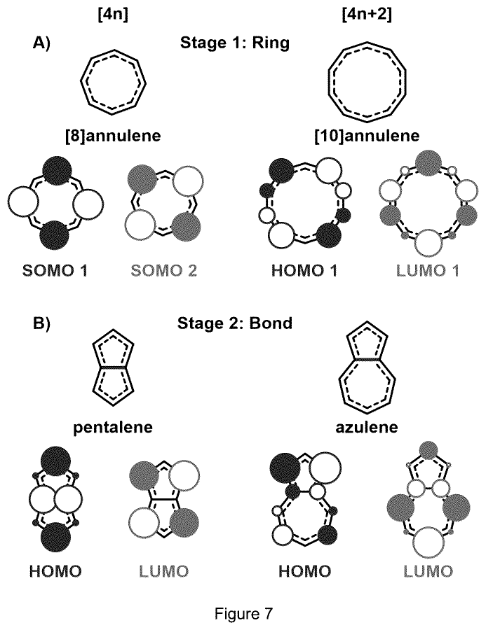

Accordingly, the bottom-up design rules were created to fulfill these requirements and we will rationalize each stage of the RiBS ruleset in the following paragraphs. The first stage (“ring” stage) is to start with alternant monocyclic hydrocarbons. Rings with an even number of members were used so that an even number of electrons in the π-system was used and a higher symmetry achieved which makes the appearance of orbital nodes at, or at least close to, nuclei more likely. In alternant monocyclic hydrocarbons with 4n π-electrons, the two degenerate non-bonding SOMOs can be chosen to be perfectly disjoint and have orbital nodes located precisely at the nuclei. 100 One of the SOMOs will then have the orbital nodes only at the starred nuclei, the other only at the unstarred nuclei. This is precisely the orbital appearance needed to violate Hund's first rule. While monocyclic hydrocarbons with 4n+2 π-electrons do not fulfill this requirement perfectly, the effects of the other stages of the ruleset can still cause violations of Hund's first rule. Illustrations of the corresponding relevant frontier orbitals for two exemplary systems are provided in FIG. 7A and are based on the Hückel approximation as implemented in HuLis.¹⁰¹⁻¹⁰³

In the second stage (“bonds” stage) of the bottom-up design, which is an optional stage, bonds between two starred or between two unstarred centers of the alternant monocyclic hydrocarbons are introduced. Consequently, the resulting structural frameworks become non-alternant polycyclic hydrocarbons. 3-membered rings were disregarded due to their reactivity and ring sizes larger than 8 due to the ensuing possibility for double bond stereoisomerism. When starting with alternant monocyclic hydrocarbons, as mentioned in stage 1, π-systems with either two degenerate HOMOs (4n+2 π-electrons) or two degenerate SOMOs (4n π-electrons) were obtained. These orbitals can be chosen to be at least partially disjoint in the former case or to be perfectly disjoint in the latter. When introducing bonds between either two starred or two unstarred centers, the symmetry of the system is reduced, and the degeneracy of the frontier orbitals is lifted. Lifting the degeneracy is particularly important for the 4n π-systems as it raises the energy of the T₁ state to be closer to the S₁ state than to the S₀ state in the resulting system. It also raises the energy of S₁ and brings the corresponding excitation energy (closer) to the visible light region. Additionally, for 4n π-systems, the bonds are always introduced between two centers that have non-zero orbital coefficients in only one of the two SOMOs which means that this SOMO is stabilized significantly without affecting the disjointness between these two orbitals considerably. For 4n+2 π-systems, this is usually not the case. However, in these systems, the newly introduced bonds reduce the symmetry of the π orbitals and increase the disjointness of the frontier orbitals. The newly introduced bonds lead to the stabilization of π-orbitals promoting bonding interactions between the newly connected centers and destabilization of π-orbitals promoting antibonding interactions between these atoms. Thus, from the original two sets of degenerate frontier orbitals, the resulting HOMO has an antibonding interaction between the newly connected atoms and the resulting LUMO has a bonding interaction between them. These are also the two orbitals with the highest degree of disjointness in the original degenerate sets. The effect of introducing bonds between either two starred or two unstarred centers into the original alternant monocyclic hydrocarbons is illustrated in FIG. 2B.

In the third and final stage (“substitution” stage) of the RiBS ruleset, which again is optional, a subset of either only starred or only unstarred methine groups is substituted with donor groups and a subset of the other set of methine groups is substituted with acceptor groups. If the second stage of the ruleset is skipped, one consequence of this type of substitution will be to lift the degeneracy in the HOMOs and SOMOs of the original alternant monocyclic hydrocarbons. As discussed previously, this is important for the 4n π-systems as it raises the energy of the T₁ state which is otherwise relatively close to S₀. It also raises the energy of the S₁ state to bring the corresponding excitation energy (closer) to the visible light region. Nevertheless, the main purpose of the substitutions is to increase the disjointness between the HOMOs and LUMOs in the resulting structures. As a side effect, substitutions also tune the HOMO-LUMO gaps and thus the excitation energies between S₀ and S₁. As the HOMOs and LUMOs are at least partially disjoint, introducing substituents at the starred positions will mainly affect the energies of one of the two frontier orbitals and substituents at the unstarred positions will mainly affect the energies of the respective other. Generally, the introduction of electron-withdrawing substituents will stabilize orbitals with large orbital coefficients at the substitution sites and diminish the corresponding orbital coefficients. Accordingly, the introduction of electron-donating substituents will destabilize orbitals with large orbital coefficients at the substitution sites and increase the corresponding orbital coefficients. When incorporating substituents with a mesomeric effect, that influence can be disentangled from their (potential) inductive effect. The added conjugation generally tends to decrease the orbital coefficients at the substitution sites. Additionally, it tends to increase the energy of HOMOs and decrease the energy of LUMOs leading to reduced HOMO-LUMO gaps. Apart from the mesomeric effect, the inductive effect of substituents that also introduce conjugation is still determined by whether they are overall electron-donating or electron-withdrawing and equivalent in nature as described above. Importantly, all these substituents also have an influence on the orbital coefficients further away from the substitution site. Thus, substitution can be used to finetune both orbital coefficients and excitation energies in the resulting molecules to maximize disjointness between HOMOs and LUMOs.

As has been discussed extensively in the literature, it is important to realize that the introduction of both electron-donating and electron-withdrawing substituents at distinct sites into a given structure leads to the stabilization of antiaromatic π-systems and to the destabilization of aromatic π-systems.¹⁰⁴ It is commonly referred to as the so-called push-pull effect.¹⁰⁵ This has been demonstrated quantitatively by investigating the influence of donor-acceptor substitution on HOMO-LUMO gaps^{104,106,107} and resonance energies^{104,105,107,108} in various cyclic conjugated π-systems. Both these properties have been regarded as measures of aromaticity and stability where higher property values usually correspond to higher stability and higher aromaticity.¹⁰⁴ Accordingly, the push-pull effect leads to an increase of both HOMO-LUMO gaps and resonance energies in antiaromatic π-systems but to a decrease of both these properties in aromatic ones.¹⁰⁴ Simultaneously, the push-pull effect leads to an increase of π-electron delocalization in otherwise antiaromatic compounds.^{104,109,110} Hence, the third and final stage of RiBS also allows to stabilize otherwise unstable π-systems such as cyclobuta-1,3-diene, cycloocta-1,3,5,7-tetraene and pentalene, and this has been utilized very successfully to synthesize, isolate and characterize stable analogs of many, otherwise unstable, and reactive structures (vide infra). Notably, the push-pull effect for stabilization of π-systems is closely related to the concept of topological charge stabilization, which is effectively just an alternative and more intuitive way for its rationalization and allows to use it in a more systematic and general way.¹¹¹

Importantly, the methine group substitutions explored in this work were intentionally restricted to limit the number of molecules to be simulated. Furthermore, the incorporation of additional π-substituents as linkers between the core structure and electron-donating or electron-withdrawing groups has also been neglected and it is expected to be particularly effective to effect higher OSs while maintaining inverted STGs as shown in previous work.¹⁹ This demonstrates that the number of potential organic INVEST molecules is considerably higher than what was exemplified here. Notably, one modification that could increase the diversity of INVEST structures significantly is the incorporation of boron into the cyclic systems, which has been demonstrated recently for the design of new structures with inverted STGs.¹⁰ However, it would also require special consideration to maintain molecular stability and is not as straightforward as the incorporation of nitrogen as explored here. Finally, there is one construction rule for INVEST core structures demonstrated previously that we did not incorporate, i.e., bridging two methine groups that are connected to a common trigonal carbon within a π-system with an ethylene bridge.¹⁹ Notably, this bridge either connects two starred or two unstarred atoms from the RiBS ruleset. With this additional construction rule, the space of molecules would be expanded from solely ortho-fused ring systems to include peri-fused ring systems as well. However, as this rule would increase the number of potential families to be explored dramatically, we decided it to be outside the scope of this work.

Virtual Screening. The methodology employed in this work is motivated by extensive investigations in the literature that showed the necessity of excited state simulation approaches accounting for double excitations to model the properties of compounds with inverted STGs.^{13,14,18-22,112-117} Thus, methods were selected that are both able to account for double excitations and are still computationally efficient enough to be used in high-throughput virtual screening for almost 70,000 small organic compounds consisting of up to 41 atoms. In particular, ADC(2),⁵³-66 CC2,^{63,65,66,68,69} CIS(D),^{61,63,66-68,70-72} and both double-hybrid³⁷ and spin-flip³⁹⁻⁴⁴ time-dependent density functional approximations (TD-DFAs) were employed which have been shown previously to be reasonable approaches to model the excited states of molecules with inverted STGs.^{13,14,19-21,112,116} While particular results for a single compound can differ and yield diverging predictions regarding the energetic order of the first excited singlet and triplet states, the general observation that the bottom-up design rules established in this work increase the probability of discovering organic INVEST molecules holds across most of the multitude of approaches tested. Additionally, extensive comparisons of all the methods employed, which are found in Example 4 Supporting Information, show that mutual agreement between the alternative theoretical approaches is reasonable supporting the main conclusions. Nevertheless, based on previous literature,^{13,14,19-21} out of all the approaches tested, ADC(2) is expected to provide overall the most reliable description of the excited state properties and, thus, the corresponding results were discussed extensively in the main text. Notably, EOM-CCSD simulations were also performed for all the compounds as it is considered to be even somewhat more reliable than ADC(2) to account for double excitations in the excited state.¹¹⁸⁻¹²⁶ However, they turned out to be too demanding computationally and thus had to be deferred.

Molecular Families. Next, the 16 molecular families were investigated separately in more detail to assess whether relevant experimental evidence pertaining to their excited state properties has been reported before and whether they are viable compounds for organic electronic materials in terms of stability and synthesizability.

Family I: Cyclobuta-1,3-dienes. Cyclobuta-1,3-diene is the archetype of the antiaromatic compound with 4 π-electrons, and its high inherent ring strain prevents it from significant deformation from planarity. Accordingly, huge efforts, both in theory and experiment, have been dedicated to its isolation, characterization and to understand its properties, as discussed in several reviews and book chapters over the years.¹²⁷⁻¹³⁰ For a long time, cyclobuta-1,3-diene derivatives remained elusive due to their inherent reactivity and instability. Nevertheless, the immense efforts dedicated to isolation of the parent compound resulted in the development of various synthetic routes that allow its generation, at least intermittently, as reactive intermediate, which immediately dimerizes at higher temperatures and can only be isolated in cold matrices.¹³¹⁻¹³⁶ Several decades later, a seminal paper demonstrated the generation and characterization of cyclobuta-1,3-diene inside a hemicarcerand at ambient temperature.¹³⁷ Additionally, several substituted derivatives were prepared^{134,138-142} which allowed extensive structural characterization and confirmation of the theoretically predicted square planar geometry in the ground-state of cyclobuta-1,3-dienes.¹⁴³⁻¹⁴⁶ Notably, even sterically hindered analogues showed considerable reactivity.¹²⁷ As an alternative to stabilization of cyclobuta-1,3-dienes using bulky substituents, push-pull substituted analogues have been studied in detail^{107,147,148} and stable analogues were successfully isolated.¹⁴⁹⁻¹⁵³ However, despite significant stabilization, these compounds were still reactive in solution. Finally, nitrogen-substituted derivatives of cyclobuta-1,3-dienes have also been investigated extensively as the introduction of nitrogen was predicted to provide significant stabilization.¹⁵⁴ Accordingly, both bulky and push-pull substituted azacyclobuta-1,3-dienes were isolated and fully characterized despite their still relatively low stability.¹⁵⁵⁻¹⁵⁹ This shows that while the unsubstituted cyclobuta-1,3-diene is very reactive, introducing appropriate substituents allows to effect significant stabilization. Recently, a BN-substituted isoelectronic substituted analog of cyclobuta-1,3-diene has been successfully synthesized and characterized and it was predicted to possess an STG of only 0.10 eV, which agreed acceptably with experimental observations.¹⁶⁰ Overall, the immense body of literature on cyclobuta-1,3-dienes also shows how academic interest in a particular class of compounds sparks considerable efforts in synthesis even if the prospects of success seem slim from the outset. Nevertheless, the remaining reactivity of even the most stable cyclobuta-1,3-dienes suggests that they are likely not very promising candidates for organic emitters.

Family II: Benzenes. Benzenes are by far the most well-known molecules covered in this study, largely due to their exceptional stability explained by their aromaticity. While emitter candidates were found with as low a STG as 0.21 eV, it seems unlikely it can be inverted with simple subsequent substitutions. Notably, several thermally activated delayed fluorescence emitters based on benzene with even lower STG are known,¹⁶¹⁻¹⁶³ however, they are designed based on minimizing overlap between HOMOs and LUMOs by connecting donor and acceptor π-systems that are orthogonal to each other, which is unlikely to induce significant spin polarization.

Family III: Cycloocta-1,3,5,7-tetraenes. After cyclobuta-1,3-dienes, cycloocta-1,3,5,7-tetraenes are the second most well-known formally antiaromatic compounds and they have 8 π-electrons. However, while the 4-membered ring is too constrained for cyclobuta-1,3-dienes to distort considerably from planarity, the 8-membered ring prefers departure from planarity which significantly reduces angle strain and diminishes conjugation avoiding antiaromaticity.^{95,96,164} This is reflected in the simulated geometries of the cycloocta-1,3,5,7-tetraene derivatives studied and can be directly gleaned from the comparably small planarity descriptors (cf. FIG. 4). Consequently, cycloocta-1,3,5,7-tetraenes are non-aromatic,^{164,165} much more stable than cyclobuta-1,3-dienes and both isolation and characterization of the parent compound was achieved already more than 100 years ago via classical synthesis.¹⁶⁶ Notably, cycloocta-1,3,5,7-tetraene has even been isolated from specific fungi showing it to be a biotic material.¹⁶⁷ A large variety of synthesis methods for cycloocta-1,3,5,7-tetraenes have been developed since their first isolation and both their synthesis and reactivity has been studied extensively.¹⁶⁸ Two recent reviews provide a comprehensive overview of some of the most important synthetic approaches^{169,170} demonstrating the high accessibility of cycloocta-1,3,5,7-tetraenes. Additionally, various aza-analogs have been synthesized in the literature, specifically monoaza-,¹⁷¹⁻¹⁸⁰ diaza-,^{159,181-193} triaza-,¹⁹⁴⁻¹⁹⁶ and tetraaza-derivatives,¹⁹⁷⁻²⁰³ showing that a large variety of candidates for organic electronic materials are accessible in this structural family. Furthermore, cycloocta-1,3,5,7-tetraene, and substituted derivatives thereof,²⁰⁴ have been used as triplet-state quenchers in lasers²⁰⁵⁻²⁰⁹ and octasubstituted analogs were demonstrated to be effective electron transporting materials in organic light-emitting diodes.²¹⁰ This suggests that molecules in this structural class are generally suitable for practical applications as organic electronic materials. Notably, a recent in-depth study of the vacuum UV spectrum of cycloocta-1,3,5,7-tetraene in combination with extensive simulations revealed the presence of several low-lying doubly excited electronic states which is important regarding the potential of substituted analogs to possess inverted STGs as predicted in this work.²¹¹ Thus, this molecular family has significant potential for future organic emitters.

### Family IV: Pentalenes

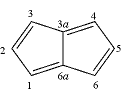

As all the remaining families studied in this work, pentalenes are non-alternant hydrocarbons. The parent compound, pentalene, has 8 π-electrons and is anti-aromatic^{212,213} making it very reactive, even towards itself,²¹⁴ and it has only been isolated and characterized spectroscopically in an argon-nitrogen matrix at approximately 20 K after photolysis of its dimer.²¹⁴⁻²¹⁶ In 1962, hexaphenylpentalene was the first substituted analogue found to be isolable and thermally somewhat stable, largely attributable to its steric hindrance.²¹⁷ While unreactive in the solid state, it is sensitive towards oxygen in solution. Following that success, other sterically hindered analogues of pentalene were isolated as well, which allowed their crystallographic structure determination.^{218,219} Several years later, in 1967, 1,3-bis(dimethylamino)pentalene was the first push-pull substituted pentalene to be isolated,²²⁰ followed by several others in the subsequent decades.^{202,221-227} Importantly, this demonstrates that push-pull substituted pentalenes show remarkable stability and synthesizability, at least compared to the parent compound, making this family attractive as potential organic INVEST emitters, especially when further stabilization is achieved. Notably, some of these push-pull substituted pentalenes were also shown to be emissive in solution.²²¹ However, these derivatives seem to emit from the second rather than the first excited state and, hence, violate Kasha's rule,²²⁸ an observation that seems to have remained unnoticed in the literature, even by the original authors. Without referring to this finding, an explanation has been provided more than 20 years later by predicting a low-energy conical intersection between S₀ and S₁ in unsubstituted pentalene that is accessible on the S₁ surface, which likely is preserved in the push-pull substituted derivatives.²²⁹ This is important as efficient nonradiative decay of singlet excitons causes very low quantum yields in potential emitters. Hence, particular attention needs to be dedicated to engineering the position of the S₀/S₁ conical intersection in pentalenes to make them better candidates for organic emitters. Notably, the violation of Kasha's rule in substituted pentalene was rediscovered experimentally more than 25 years later after the first report without mentioning it.²³⁰ Curiously, the authors of that study also missed the computational prediction of S₀/S₁ conical intersections in pentalenes and attributed the violation of Kasha's rule to the large energy gaps between excited singlet states.^{231,232} Finally, a few isoelectronic BN-substituted analogues of pentalene have also been successfully isolated and characterized,²³³⁻²³⁵ one of which even with respect to its excited state properties but it had a relatively large STG.²³⁵ Notably, based on PPP simulations, pentalene itself has previously been proposed to have an inverted STG in its non-distorted structure with D₂ₕ symmetry.⁴ However, in the preferred ground-state geometry, its STG is not inverted. This is confirmed by our computation as most of our predictions for the corresponding STG are between 0.8 eV and 0.9 eV.

### Family V: Azulenes

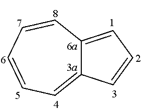

Apart from benzenes and cycloocta-1,3,5,7-tetraenes, azulenes constitute the third very well-known family of molecules investigated in this work. Azulene is a non-alternant aromatic multicyclic system with 10 π-electrons.^{212,213,236,237} Thus, azulene is stable and substituted derivatives are also found in nature.²³⁸ These natural products have been isolated long before the publication of the first total synthesis of a derivative in 1939.²³⁹ Azulenes have been of great interest to the scientific community for more than 100 years owing to both their unusual core structure and their unique properties. Consequently, a wide range of synthetic methods have been developed, the most important of which have been reviewed extensively.^{238,240-242,242-247} The exceptional electronic properties of azulenes have resulted in numerous applications for both optical and electronic materials which are summarized in several recent reviews.²⁴⁸⁻²⁵⁹ One of these exceptional properties is that azulene shows its highest fluorescence intensity from the second excited singlet state,^{260,261} i.e., S₂, thereby breaking Kasha's rule.²²⁸ The currently accepted explanation for that phenomenon is the existence of an energetically accessible conical intersection between the first excited singlet state, i.e., S₁, and the ground state that causes excitons in S₁ to decay radiationlessly.²⁶²⁻²⁶⁵ Notably, this is an undesired property for organic light-emitting diode materials as there are likely several excited triplet states located between the S₁ and S₂ energies resulting in very large singlet-triplet gaps in devices. However, not all azulene derivatives exhibit this property and substituted analogs with dominant emission from S₁ have been demonstrated to exist. In particular, derivatives with carbonyl substituents in conjugation with the central ring system²⁶⁶⁻²⁷⁰ and 1,3-diazaazulene²⁷¹⁻²⁷⁴ were shown to exhibit fluorescence from S₁. This demonstrates that careful molecular design can avoid the breaking of Kasha's rule in azulenes which is important for their potential as organic emitters. A less well-known exceptional property of azulene is its extremely small singlet-triplet gap of only 0.049 eV as determined recently by anion photoelectron spectroscopy,²⁷⁵ which agrees well with previous experiments²⁷⁶⁻²⁷⁹ and simulations,^{275,280} and also with the present results. This small gap is the basis for the realization of azulene derivatives with inverted STGs by substituents enhancing dynamic spin polarization to stabilize the first excited singlet states relative to the first excited triplet states. In comparison to azulenes, azaazulenes with nitrogen atoms directly incorporated into the core structure have been significantly less explored. However, compared to most of the other molecular families considered herein, a considerable amount of prior research exists, and two relatively recent reviews provide an overview about their syntheses and reactivities.^{281,282} Overall, azulenes seem to hold great promise as potential future INVEST emitters given their stability, synthetic accessibility, and their favorable predicted excited state properties. However, a special emphasis should be placed on making their S₁ states emissive by avoiding nonradiative decay.

### Family VI: Bowtienes

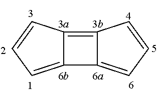

Bowtiene is non-alternant, also has 10 π-electrons and, at least based on resonance energy estimations, is considered either non-aromatic^{236,237} or at least somewhat aromatic.²⁸³ To the best of our knowledge, neither bowtiene, nor any substituted analogues of bowtiene have ever been synthesized, despite several reported forays and attempts.²⁸³⁻²⁸⁵ Notably, the absence of resonance energy stabilization even led to the prediction that synthetic attempts would be in vain,²³⁶ which so far seems to stand. Accordingly, while push-pull substituted analogs might be more stable than the parent bowtiene, overall, this family does not seem to be a promising target in terms of synthesizability, despite it likely being stable towards unimolecular rearrangement or dimerization.⁸⁷

### Family VII: Heptalenes

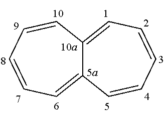 [SMILES: C1=CC=C2C=CC=CC=C2C=C1]

Heptalenes are the first family to be discussed with 12 π-electrons, are non-alternant, and, based on resonance energy estimations, are considered non-aromatic or at least somewhat anti-aromatic.^{236,237,286-288} This is also reflected in their significant double bond localization,²⁸⁹⁻²⁹¹ their largely non-planar structure^{97,98} and the resulting potential of substituted analogs for persistent helical chirality.²⁹²⁻²⁹⁵ The isolation of heptalene and its characterization has been reported already in 1961 and the unsubstituted parent compound has been shown to be very sensitive to oxygen or heat,²⁹⁶ in accordance with predictions.²⁹⁷ Nevertheless, the successful isolation using ordinary synthetic techniques shows its significantly higher stability compared to pentalene. Symmetric 3,8-disubstitution has been shown to provide significant stabilization allowing isolation, crystallization and inducing considerable thermal stability.^{298,299} To the best of our knowledge, only one derivative with a nitrogen incorporated into the ring system has been reported on, and its instability precluded isolation and full characterization.³⁰⁰ However, not only donor-acceptor substituted heptalenes³⁰¹⁻³⁰⁴ but also substituted heptalenes in general are stable which sparked comprehensive exploration of their syntheses, structures and properties in the past few decades unveiling a rich chemical family.³⁰⁵⁻³³⁰ This wealth of synthetic studies towards substituted heptalenes makes them synthetically readily accessible, and together with the high stability of substituted analogues renders this molecular family particularly appealing as potential INVEST emitters. Notably, previous PPP simulations predicted the parent heptalene to have an inverted STG at a non-distorted geometry with D₂ₕ symmetry but not at its preferred ground-state geometry.⁴ The present simulations confirm the absence of STG inversion at its ground-state geometry and suggest it to be between 0.6 eV and 0.7 eV.

### Family VIII: Zurlenes

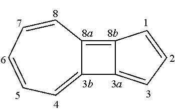 [SMILES: C1=CC=C2C3=CC=CC3=C2C=C1]

Zurlene is non-alternant, also has 12 π-electrons and based on resonance energy estimations is considered at least partially aromatic,^{236,237} although one early study argued for it to be antiaromatic.⁹³ Neither zurlene, nor any of its derivatives have been isolated to date, and only one publication has reported synthetic forays towards that target.⁸⁸ Nevertheless, its stability and reactivity has been studied computationally⁸⁶ and zurlene was proposed to be “reasonably stable”.³³¹ However, the notorious absence of publications on zurlenes or its derivatives makes a proper evaluation of both synthesizability and stability impossible, as the absence of reported syntheses generally does not mean a compound is not synthesizable, especially because only one failed synthesis has been reported on. Nevertheless, it makes zurlenes not a particularly promising target family for immediate follow-up studies.

### Family IX: s-Indacenes

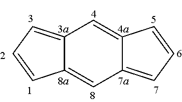 [SMILES: C1=Cc2cc3c(cc2=C1)C=CC=3]

Like heptalene and zurlene, s-indacene is non-alternant, consists of a 12 π-electron system and is considered non-aromatic based on resonance energy calculations.^{236,237,286} Based on its other properties, s-indacene is considered to have mixed aromatic and anti-aromatic character and can be considered a prototypical compound to separate the classes of aromatic and antiaromatic compounds.^{332,333} While the parent compound is very reactive and, hence, has only been obtained at low temperatures due to its susceptibility to disproportionation,^{109,110} several substituted derivatives were successfully isolated at ambient conditions due to their increased stability when functionalized at specific positions.²⁹⁷ The first three s-indacenes to be isolated were 2-(tert-butyl)-4,8-bis(dimethylamino)-s-indacene, 2,6-(di-tert-butyl)-4,8-bis(dimethylamino)-s-indacene and 2,6,8-(tri-tert-butyl)-4-(dimethylamino)-s-indacene, which are stabilized both sterically and electronically and at least the two bis(dimethylamino)-substituted derivatives were shown to be persistent under both heat and air even during prolonged exposure.^{224,334} Importantly, similar to the stabilized pentalenes discussed previously, this demonstrates the strong stabilization provided when the right substituents are introduced at the right positions and should be seen as strong encouragement that even seemingly unstable π-systems, like many others discussed in this work, should be synthetically accessible despite their absence from the literature. The difference in stabilities between the bis(dimethylamino)-substituted derivatives and the mono (dimethylamino)-substituted is also expressed in the crystal structures of these compounds as the former show higher delocalization in the bond lengths.²²⁴ Moreover, the stability imposed by the two amine substituents is also demonstrated by the ability of 2-(tert-butyl)-4,8-bis(dimethylamino)-s-indacene to react with electrophiles in an ordinary electrophilic aromatic substitution.²²⁴ A few years later, 1,3,5,7-(tetra-tert-butyl)-s-indacene was synthesized, isolated and its crystal structure determined, and was found to be stable as solid but sensitive towards oxygen and acid in solution.³³⁵ After its isolation, the optical spectra and excited state properties of 1,3,5,7-(tetra-tert-butyl)-s-indacene have been studied in great detail and it was found to violate Kasha's rule,²²⁸ emit light predominantly from the second excited singlet state and have a very short excited state lifetime in S₁.³³⁶⁻³³⁸ In-depth computations explained this violation and short lifetime by an S₀/S₁ conical intersection accessible from the S₁ manifold allowing fast internal conversion from S₁ to S₀ precluding significant fluorescence from S₁.³³⁹ Additionally, several heteroatom-substituted compounds incorporating nitrogen or phosphorus into the core structure have also been isolated and fully characterized demonstrating that the scope of modification for s-indacenes can be readily extended.³⁴⁰⁻³⁴³ Overall, these findings demonstrate that s-indacenes are a promising family of potential INVEST emitters but the presence of S₀/S₁ conical intersections could possibly hamper its applicability. Accordingly, modification of s-indacenes needs to be tailored towards increasing the energy of this conical intersection on the S₁ manifold.

### Family X: as-Indacenes

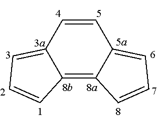 [SMILES: C1=Cc2c3c(ccc2=C1)=CC=C3]

As a very close constitutional isomer to s-indacene, as-indacene is non-alternant as well, also has 12 π-electrons and based on resonance energy estimations is predicted to be non-aromatic^{236,237} or at least partially anti-aromatic,^{286,344} and overall somewhat less aromatic than s-indacene. Accordingly, as-indacene was predicted to be less stable than s-indacene.³⁴⁵ Several reported attempts at synthesizing and isolating as-indacenes have failed delivering this class of π-systems³⁴⁶⁻³⁴⁸ and syntheses of substituted analogs have not been disseminated in the literature either. However, the intermediacy of as-indacene has been inferred in the flash vacuum thermolysis of both biphenylene and diphenic anhydride,³⁴⁹ and in the pyrolysis of benzene both with and without the addition of acetylene.³⁵⁰ As demonstrated by these observations of as-indacene as intermediate, it is shown to be very reactive, confirming theoretical predictions.²⁹⁷ Accordingly, as-indacenes do not seem to be very promising targets for potential organic INVEST emitters.

### Family XI: Anthrazulenes

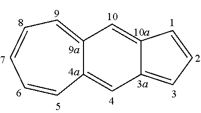 [SMILES: c1ccc2cc3cccc3cc2cc1]

Anthrazulenes are non-alternant hydrocarbons with 14 π-electrons and are predicted to be somewhat aromatic based on resonance energy estimations and reactivity predictions.^{236,237,287,288,297,331} However, despite various attempts at their synthesis and isolation, anthrazulenes have to date remained elusive.^{91,351-354} Notably, it was proposed that anthrazulene was produced as intermediate in solution but then polymerized rapidly due to its high susceptibility to electrophilic attack.³⁵² Additionally, the attempted synthesis of one azaanthrazulene was also disclosed.³⁵⁵ While it was stated that the synthesis could have been successful providing a solution showing scarlet fluorescence, the corresponding compound evaded isolation due to its instability and no proper characterization data could be obtained.³⁵⁵ Accordingly, the theoretical predictions of the aromatic stability of anthrazulenes could not be corroborated experimentally. Nevertheless, while donor-acceptor substituted analogues are likely significantly more stable, the absence of reported syntheses does not make anthrazulenes particularly attractive future targets, but it is believed that stabilized analogues should be isolable.

### Family XII: Phenazulenes

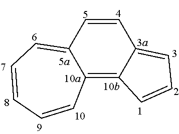 [SMILES: c1ccc2ccc3cccc3c2cc1]

Phenazulenes are non-alternant and close constitutional isomers to anthrazulenes with 14 π-electrons. Importantly, resonance energy and reactivity computations predict them to be slightly more aromatic and more stable than anthrazulenes.^{236,237,287,288,331} Similar to anthrazulene, the parent compound phenazulene was successfully generated in solution and, owing to its somewhat higher stability, could be spectroscopically characterized.^{348,356-358} However, attempts to isolate it failed causing it to polymerize, very much akin to the failure of isolating anthrazulene,³⁵² with the difference that UV-VIS absorption spectra could be recorded for phenazulene, and showed reasonable agreement to theoretical predictions.³⁵⁸ Furthermore, two 2,3-dicyano-substituted derivatives of phenazulene were successfully synthesized, isolated and fully characterized, 92 demonstrating again the power of donor-acceptor substitution to stabilize these types of π-systems. Accordingly, push-pull substituted phenazulenes are another promising family to realize organic INVEST emitters, but the sparsity of successful syntheses in the literature demands a renewal of interest towards these target compounds.

### Family XIII: Dicyclohepta [a,c] cyclobutenes

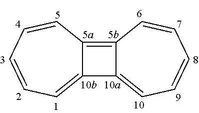 [SMILES: c1ccc2c3cccccc-3c-2cc1]

Another non-alternant 14-electron π-system studied in this work is dicyclohepta [a,c] cyclobutene. Surveying the literature only revealed a few theoretical works regarding this compound class, not even attempted syntheses of any of its derivatives have been reported. Both resonance energy estimations and geometry simulations predict the parent compound to be largely non-aromatic.^{236,237,289,359} Notably, compared to the analogous bowtiene investigated before, it is predicted to be somewhat more stable, but still reactive.^{236,237} Accordingly, dicyclohepta [a,c] cyclobutenes are likely not very promising targets for future synthetic efforts, however, if one was to prepare such a foray, push-pull substituted analogues should be targeted to increase the likelihood of success.

### Family XIV: Dicyclopenta [a,e] cyclooctenes

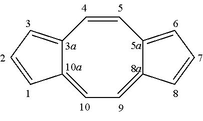 [SMILES: c1cc2ccc3cccc-3ccc-2c1]

Dicyclopenta [a,e] cyclooctenes are one of three non-alternant 14-electron π-systems investigated herein with a central 8-membered ring annulated to two 5-membered rings. Computational studies of its aromaticity are scarce compared to the other families discussed until this point, however, based on both computational and experimental evidence this class of compounds is considered aromatic.^{360,361} While the parent compound has not been reported on, a sterically protected analogue was isolated and fully characterized confirming this class of compounds to be stable. Importantly, the corresponding crystal structure revealed a high planarity of the central 8-membered ring, which is generally rare for this ring size and hints at significant stabilization due to conjugation in this class of compounds.^{360,361} A very recent computational investigation of the excited states of dicyclopenta [a,e] cyclooctene and other derivatives predicted the parent compound to have an inverted STG⁹ based on a Pariser-Parr-Pople (PPP) model Hamiltonian³⁶²⁻³⁶⁴ and the present results at the ADC(2) level of theory confirm this prediction proposing an STG of −0.01 eV. Accordingly, while the synthetic accessibility of derivatives still needs to be explored further, dicyclopenta [a,e] cyclooctenes are promising candidates for organic INVEST emitters.

### Family XV: Dicyclopenta [a,d] cyclooctenes

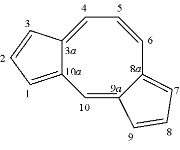 [SMILES: c1cc2cccc-2cc2cccc-2c1]

Dicyclopenta [a,d] cyclooctenes, as close analogues of the family of molecules discussed before, are also non-alternant hydrocarbons with 14 π electrons. Its aromaticity has not been studied computationally. However, the parent compound was successfully isolated and fully characterized suggesting it to be aromatic and demonstrating its high stability.⁹⁴ Additionally, the same computational study mentioned in the previous paragraph also predicted it to have an inverted STG based on a PPP model.⁹ However, our simulations at the ADC(2) level of theory predict a very small but positive STG of 0.03 eV. Nevertheless, many substituted analogs of the parent compound are predicted to have an inverted STG. Accordingly, the high stability of dicyclopenta [a,d] cyclooctenes makes them a promising target for future synthetic forays towards potential organic molecules with inverted STGs and their application as organic electronic materials.

### Family XVI: Dicyclopenta [a,c] cyclooctenes

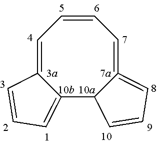

The final family of molecules to be discussed is a close constitutional isomer to the two previous ones and, hence, is yet another non-alternant 14 π electron hydrocarbon. Synthesis of the parent compound has been attempted via a key electrocyclic reaction but the desired product could not be detected.³⁶⁵ Notably, no computational studies of stability or aromaticity of this class of compounds was found but based on the observed high stabilities of the two close families of molecules discussed before, it is expected that dicyclopenta [a,c] cyclooctenes to be comparably stable. Additionally, using a PPP Hamiltonian, the parent compound was predicted to have an inverted STG, similar to the close constitutional isomers discussed before.⁹ However, unlike the other two isomers for which the present excited state simulations agreed reasonably well with the predictions based on PPP, ADC(2) was found to predict this parent compound to have an STG of 0.31 eV which is relatively small but far away from an inverted STG. Nevertheless, many substituted analogs that are predicted to possess inverted gaps were found. While we think that the expected stability of dicyclopenta [a,c] cyclooctenes makes them attractive organic emitter candidates, the absence of syntheses in the literature calls for research efforts to develop reliable procedures.

Summary. The potential of all the families discussed in the course of the present work was summarised for applications as organic emitters in Table 4 and ranked them based on their properties, including their stabilities, and their synthesizabilities given the current state of the literature. Each category was evaluated in a binary way. If there is at least one compound in a family that fulfills a certain property criterion the corresponding family will be assigned a value of 1 for that property. For STG, the predicted property should be negative to get a score of 1. For OS, the predicted property should be above 0.05. For the vertical excitation energy, the predicted property should be in the visible light region (i.e., 1.7 eV to 3.3 eV). For stability, there should exist at least one compound that is known to be stable under ambient atmosphere. For synthesizability, there should exist at least one successful synthesis report. Afterwards, the corresponding scores were summed to obtain an overall evaluation for all the compound families considered (cf. Table 4). Families III, V, VII, IX and XII receive a perfect score of 5. Thus, cycloocta-1,3,5,7-tetraenes, azulenes, heptalenes, s-indacenes and phenazulenes are the most promising compound families to be investigated more comprehensively for future applications as organic INVEST emitters. Whereas the first three of these have considerable literature precedence, the other two have remained comparably unexplored as they require careful substitution for their stabilization. Consequently, these two classes offer the most untapped potential for future research.

TABLE 4
Evaluation of performance and suitability of the investigated
families of organic emitter candidates based on their excited
state properties, their stabilities and synthesizabilities.

| | ΔE | | ΔE | | Synthesiz- | |
|---|---|---|---|---|---|---|
| Family | (S₁ − T₁) | f₁₂ | (S₀ − S₁) | Stability | ability | Overall |
| I | 1 | 0 | 1 | 0 | 1 | 3 |
| II | 0 | 1 | 0 | 1 | 1 | 3 |
| III | 1 | 1 | 1 | 1 | 1 | 5 |
| IV | 1 | 0 | 1 | 1 | 1 | 4 |
| V | 1 | 1 | 1 | 1 | 1 | 5 |
| VI | 1 | 0 | 1 | 0 | 0 | 2 |
| VII | 1 | 1 | 1 | 1 | 1 | 5 |
| VIII | 1 | 1 | 1 | 0 | 0 | 3 |
| IX | 1 | 1 | 1 | 1 | 1 | 5 |
| X | 1 | 1 | 1 | 0 | 0 | 3 |
| XI | 1 | 1 | 1 | 0 | 0 | 3 |
| XII | 1 | 1 | 1 | 1 | 1 | 5 |
| XIII | 1 | 0 | 1 | 0 | 0 | 2 |
| XIV | 1 | 1 | 1 | 1 | 0 | 4 |
| XV | 1 | 1 | 1 | 1 | 0 | 4 |
| XVI | 1 | 1 | 1 | 1 | 0 | 4 |

Validation. Initial validation was attempted by means of inspecting the normalized FOD integral of all the compounds to check whether static correlation could be relevant for a proper description of their electronic structures.⁷⁵ However, apart from finding this metric to reflect largely the energy difference between the ground state and the first excited singlet, it was found the corresponding multireference metrics to be relatively low for most of the compounds. Thus, it is contemplated that single reference quantum chemistry approaches are sufficient to describe the ground- and excited-state properties of the molecules under study.

Subsequently, the effect of geometry relaxation on the excited state properties of the S₀, S₁ and T₁ states of all the compounds studied in this work was studied. However, as geometry optimization via excited state simulation methods that account for double excitations is expensive and largely lacks efficient implementations that make use of analytical gradients, the approach was adapted to allow for high-throughput virtual screening of almost 70,000 molecules. Hence, B3LYP⁴⁶⁻⁴⁸/6-31G*⁴⁹⁻⁵¹ was used for excited state geometry optimizations as it has been shown to be a good compromise between cost and accuracy for the simulation of excited state properties of organic structures^{366,367} and it has also been employed successfully for organic INVEST compounds before.¹⁹ Nevertheless, the effect of double excitations on the corresponding excited state geometries was not accounted for. Subsequently, single point calculations were performed using the double-hybrid TD-DFA ωB2PLYP′³⁷ at the S₀, S₁ and T₁ geometries to account for double excitations at least in the electronic transitions from these structures. While ωB2PLYP′ shows a systematic offset in the STGs compared to correlated excited state methods such as ADC(2),¹⁹ which largely stems from an offset of the excitation energy between S₀ and S₁ (cf. Supporting Information), it still reproduces property trends of these correlated excited state methods sufficiently well. To be able to directly compare the magnitude of excited state property corrections obtained from ωB2PLYP′ simulations to the corresponding vertical excitation properties at the ADC(2) level, these properties were calibrated using robust linear regression via the Theil-Sen estimator to transform ωB2PLYP′ corrections into ADC(2) correction estimates.^{368,369} However, as linear correlation between the properties at the ωB2PLYP′ and ADC(2) levels is not perfect, the noise in the correction estimates which likely leads to cases where they are either significantly over- or underestimated. Nevertheless, this procedure allows for deriving general trends of the effect of geometry relaxation, especially when comparing the various compound families studied. Overall, it was found that the adiabatic STG corrections thus obtained lead to a significant increase in the number of predicted INVEST candidates. This suggests that stabilization of the S₁ state in the S₁ geometry relative to that state in the S₀ geometry tends to be larger than stabilization of the T₁ state in the T₁ geometry relative to that state in the S₀ geometry. It has been demonstrated theoretically for a four-electron model system that accounting for double excitations via spin polarization leads to a stabilization of the singlet state that is 3 times as large as the corresponding stabilization of the triplet state.¹⁶ A larger impact on energy could imply a larger impact on geometry as well that could potentially lead to a higher stabilization of the S₁ relative to the T₁.

While various excited state simulation methods were used that account for double excitations, there are some approaches considered as reliable reference methods for excited state properties that could not be employed in a high-throughput virtual screening setting. As mentioned previously, EOM-CCSD¹¹⁸⁻¹²⁶ calculations turned out to be too prohibitive to be performed on the entire set of compounds or at least on a considerable subset thereof. However, more focused computational studies with EOM-CCSD on small sets with hundreds of target compounds are perfectly feasible for the type of molecules studied in presently. Additionally, a family of methods that was not even attempted to be used as it requires significant oversight by the user are multireference electronic structure approaches such as CASSCF,³⁷⁰ CASPT2,³⁷¹⁻³⁷³ or NEVPT2.³⁷⁴ They have the potential to provide significant additional insight into the electronic structure of INVEST emitters but are only feasible in extremely focused system-specific computational studies on tens of molecules. Accordingly, it is believed that they are not very suitable for candidate selection but rather for extensive comparison of simulated against experimental excited state properties. Thus, they will be particularly valuable in future combined computational and experimental work on compound classes investigated in this work.

Currently, the systematic determination of energy-transfer rates with known triplet sensitizers was used as references to estimate triplet energies and combine the corresponding results with singlet energy estimates from optical spectroscopy which suffers from significant uncertainties.^{7,8} Recently, direct determination from both fluorescence and phosphorescence spectra has also been reported on.⁹⁹ However, that is not expected to be suitable for all INVEST compounds as phosphorescence is likely to be suppressed considerably.¹⁴

Herein, simple and clear bottom-up design rules were formulated for the construction of small organic molecules with inverted STGs. Using high-throughput virtual screening and a wide range of excited state electronic structure methods capable to account for double excitation contributions, the validity of these rules were confirmed and 15 new cyclic organic moieties identified that promote energetic inversion between the first excited singlet and triplet states. Additionally, the molecular classes' feasibility for real-world applications in terms of synthesizability, stability, and their excited state properties was investigated.

### Example 4 Supplementary Results

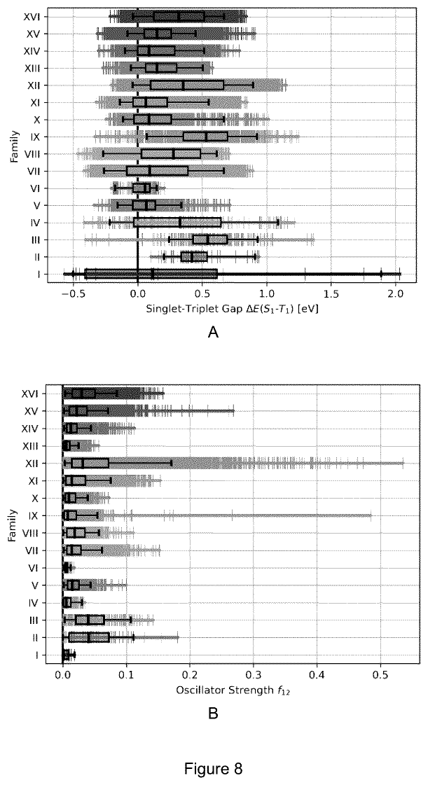

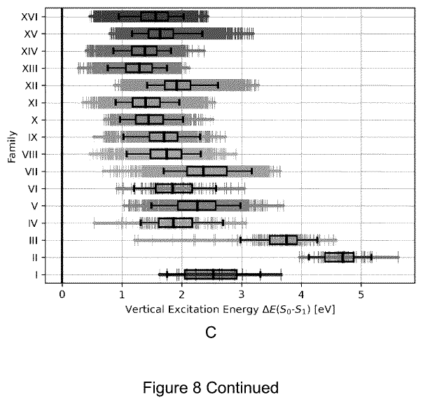

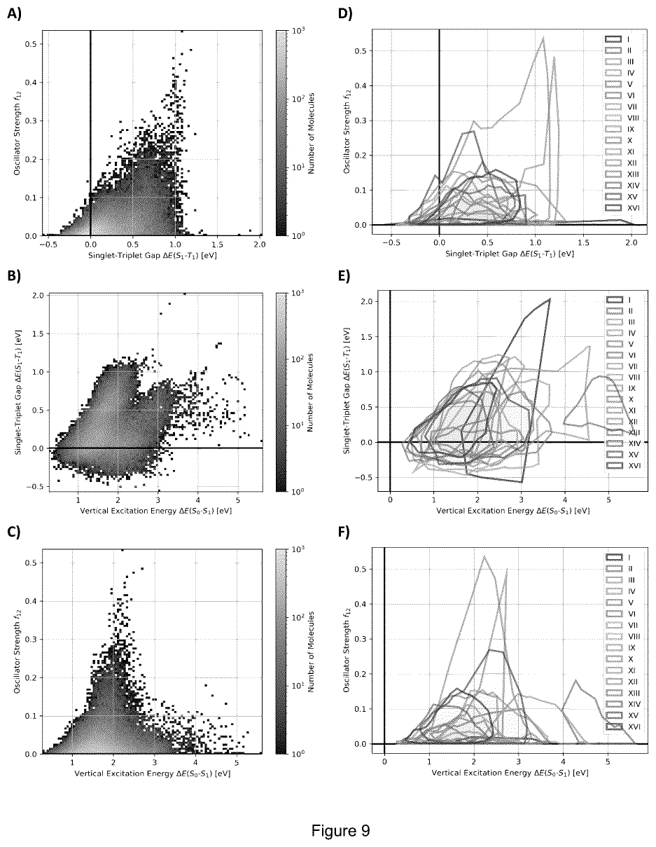

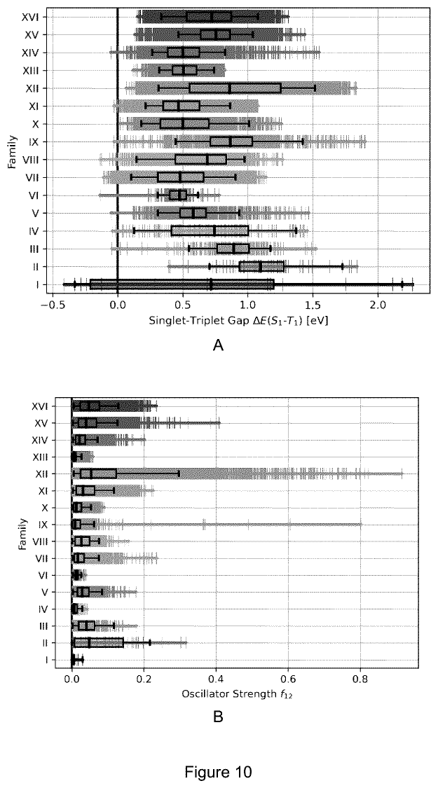

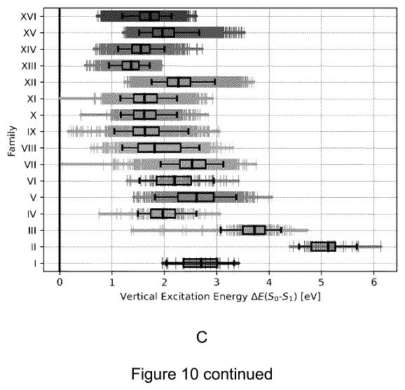

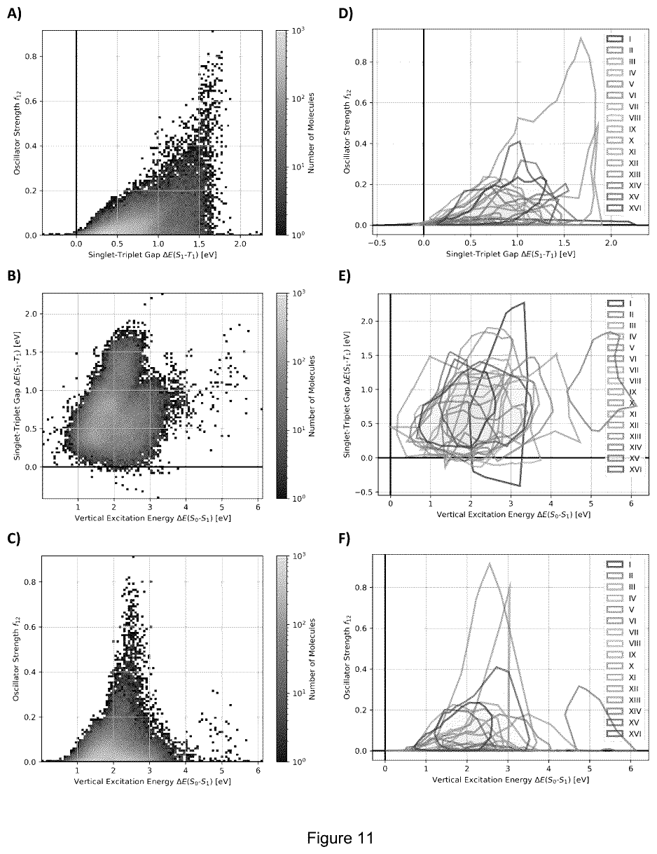

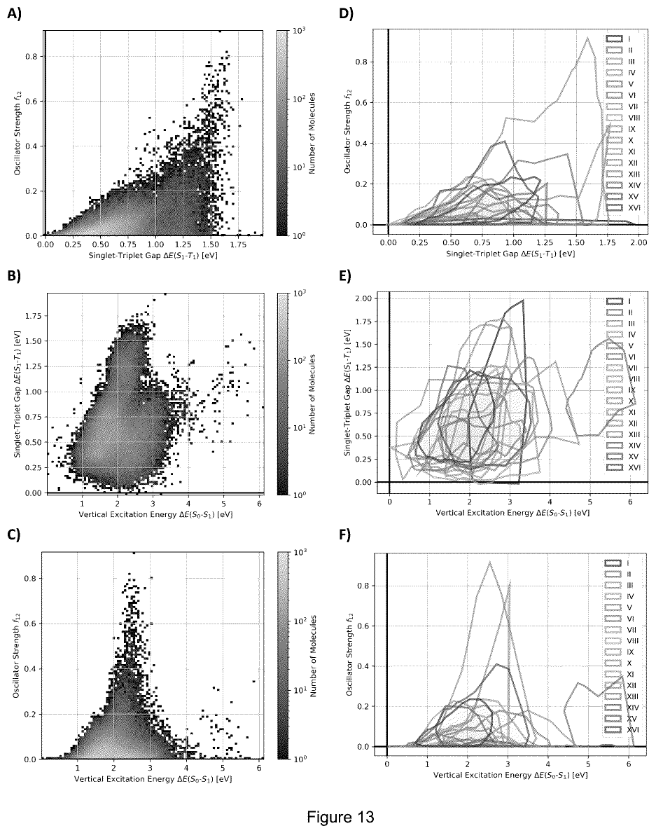

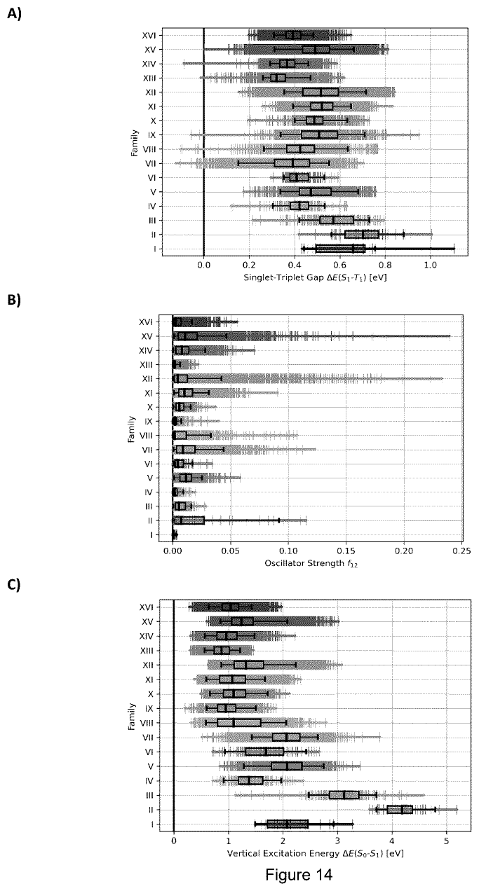

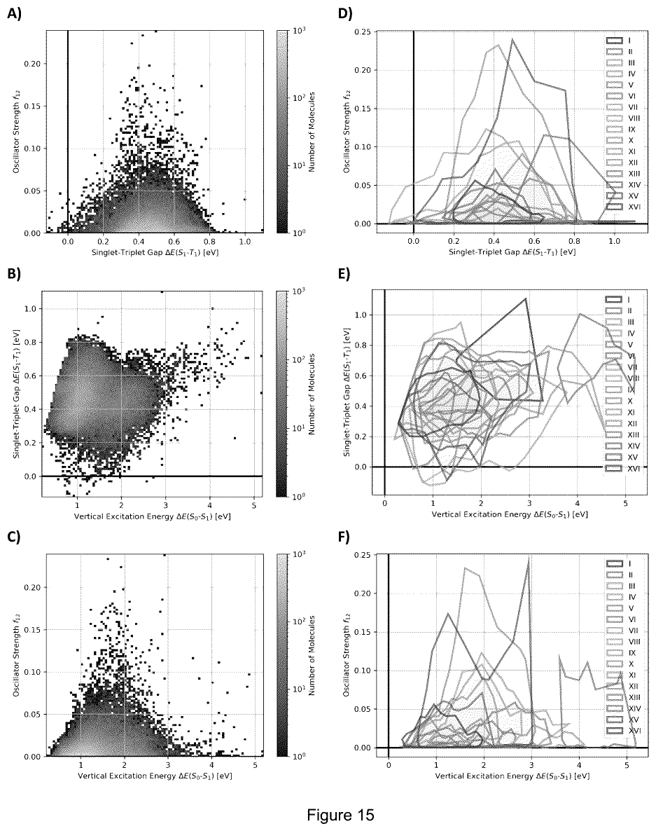

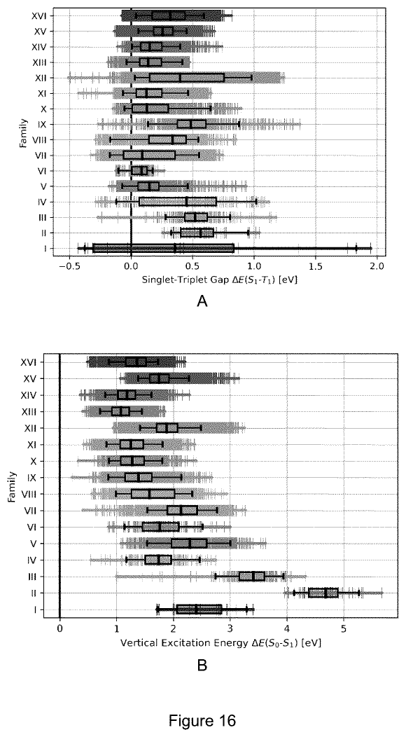

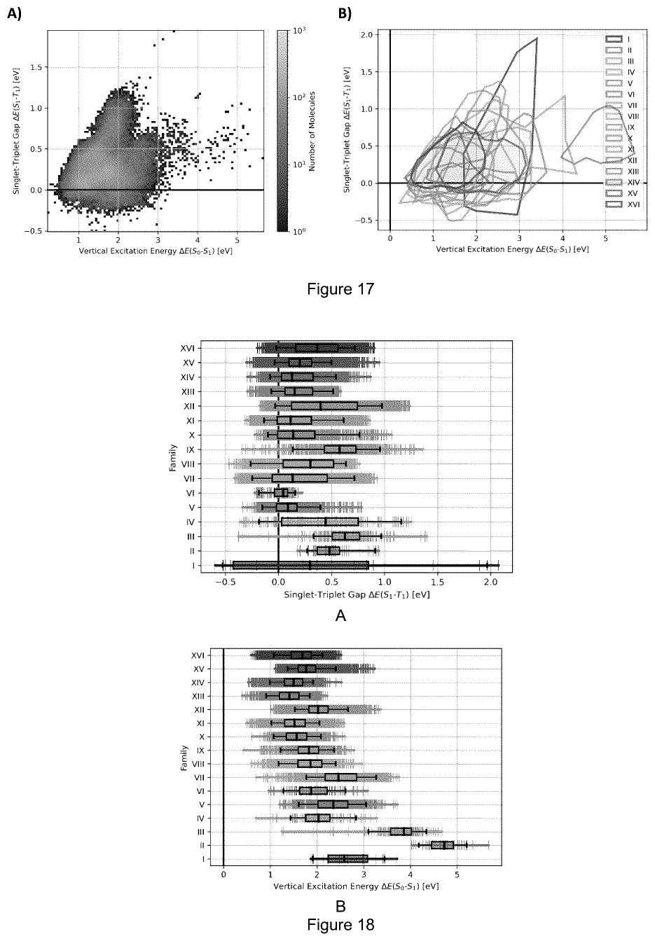

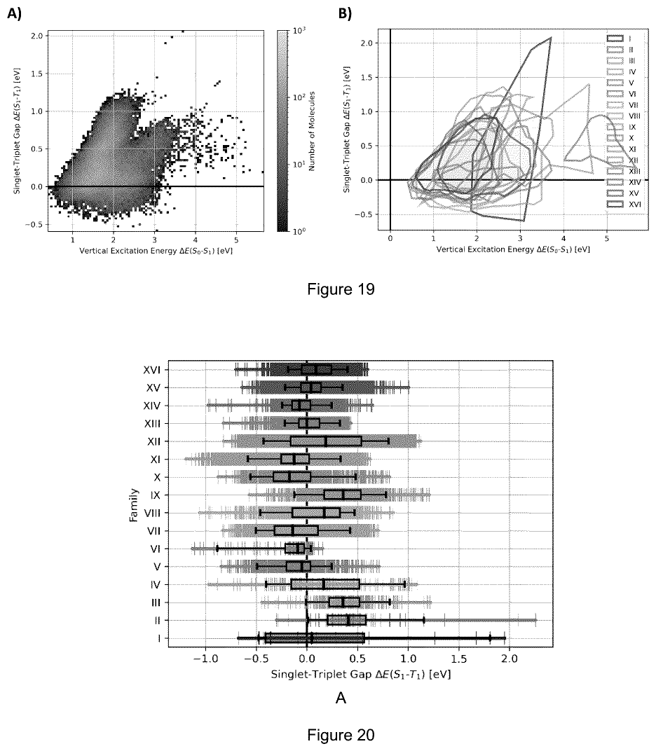

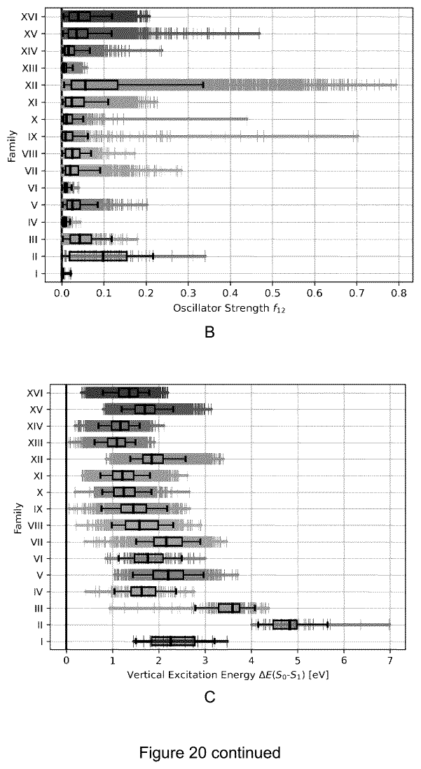

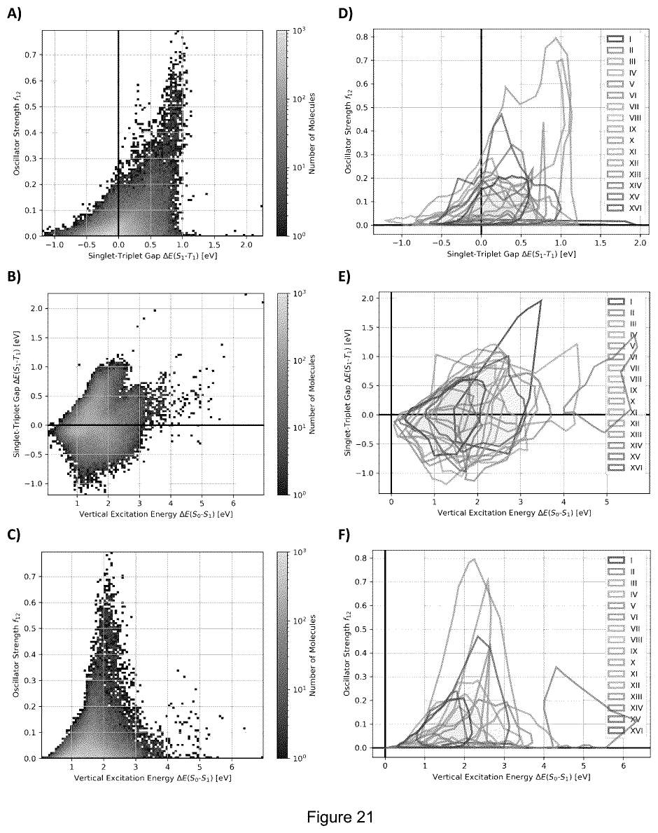

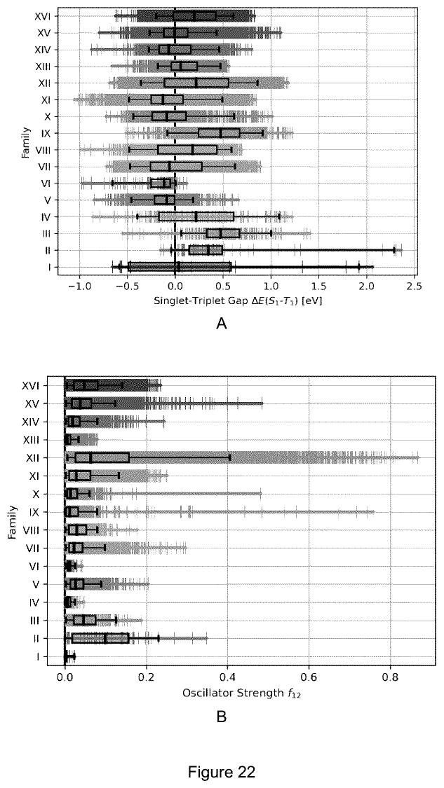

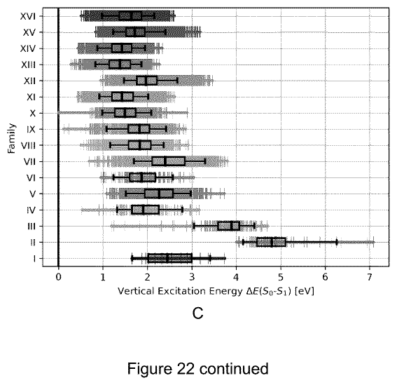

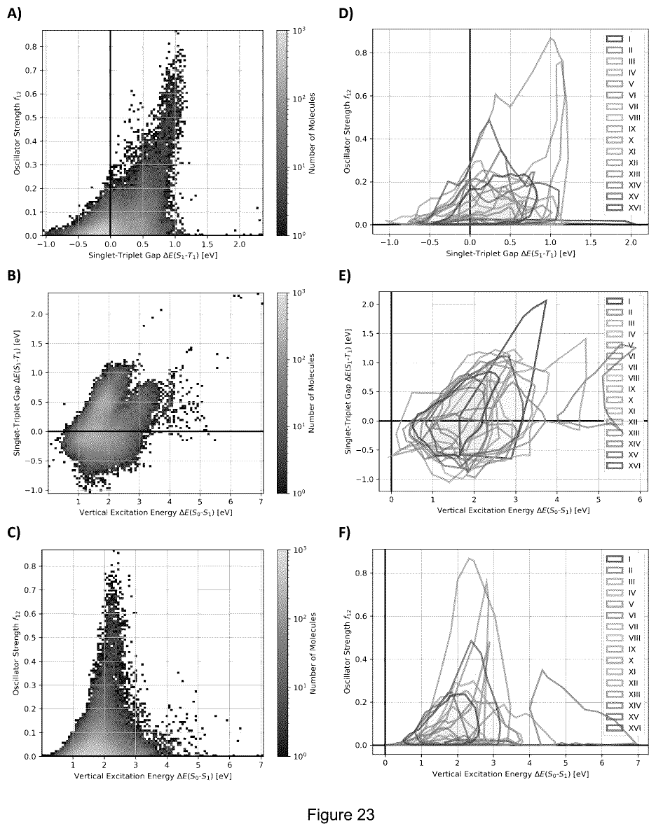

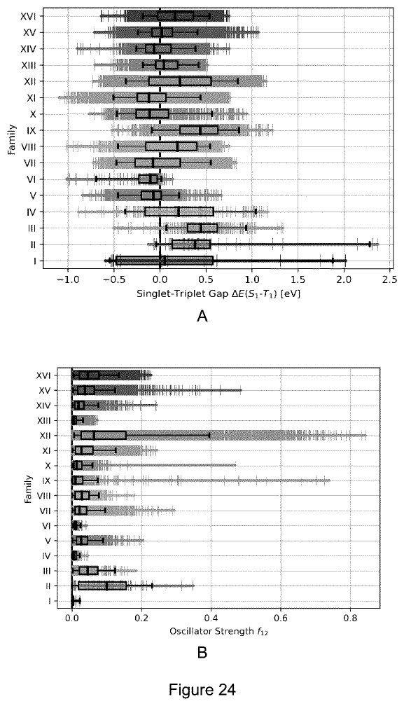

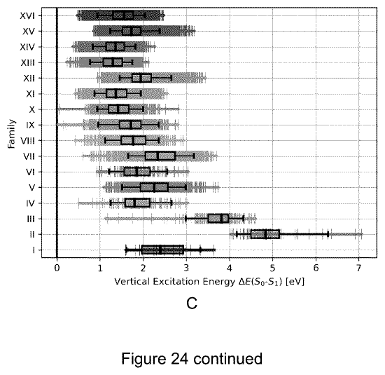

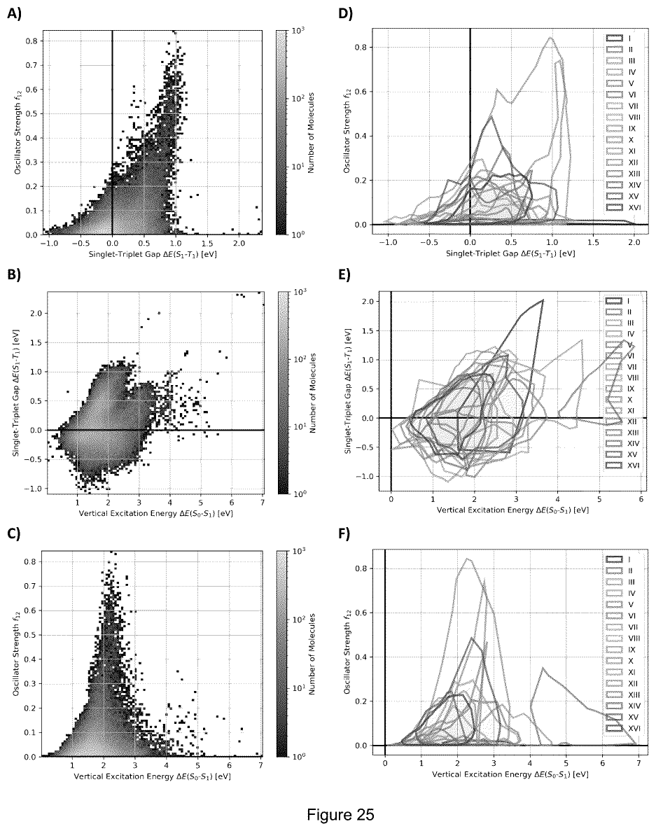

Virtual Screening. In addition to the results presented in the above examples, property distributions for all the alternative excited state simulation methods employed are provided and compared systematically to the results obtained at the ADC(2)/cc-pVDZ level of theory, which are discussed extensively in the examples above. FIGS. 8 to 25 show the predicted property ranges of the entire set of compounds at the various levels of theory, divided into the 16 compound families. In addition, they also show the corresponding histograms over all combinations of two properties. Notably, calculation of the oscillator strength was not implemented in the program used for the simulation at the CC2 and SOS—CC2 levels.

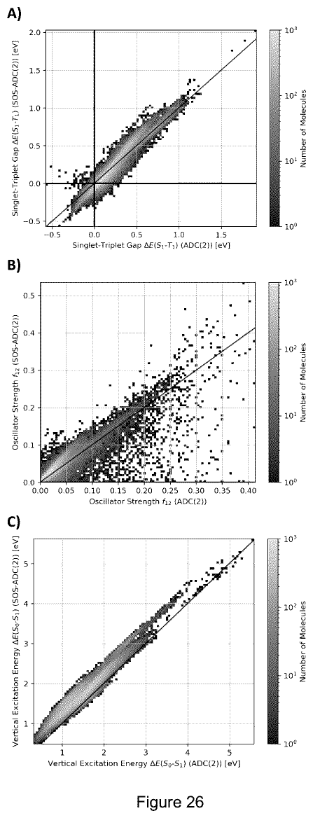

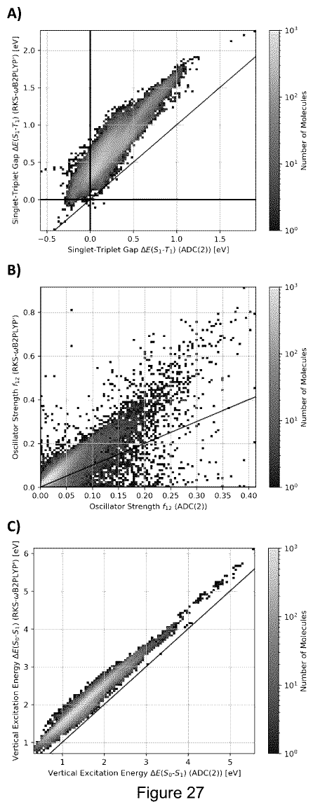

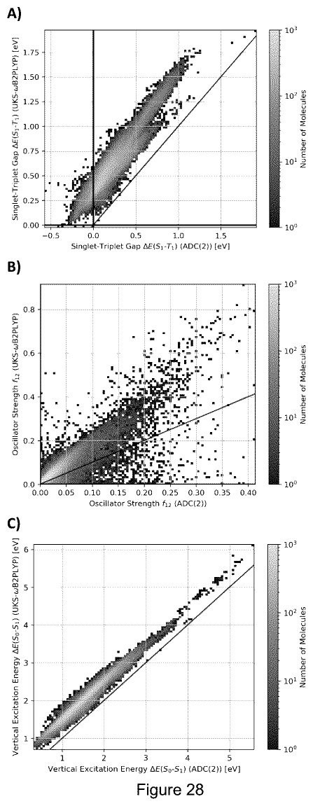

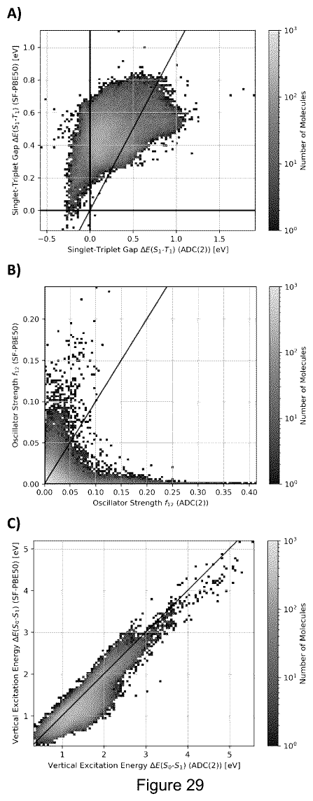

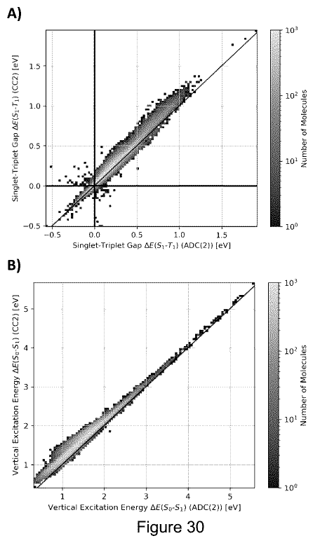

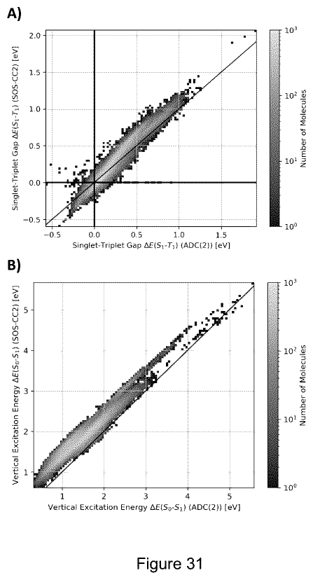

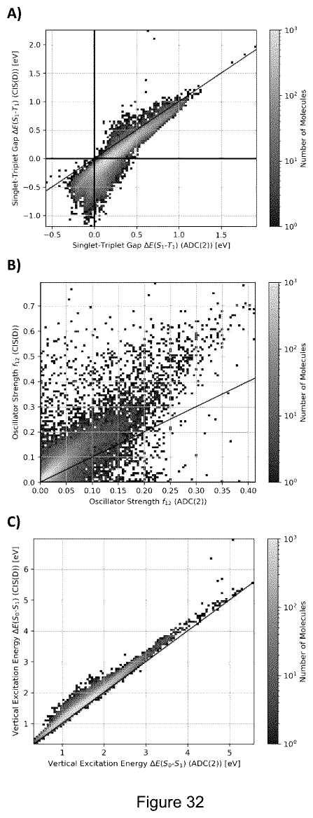

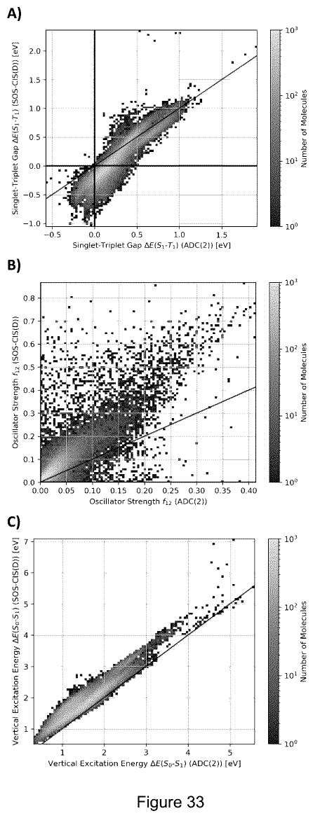

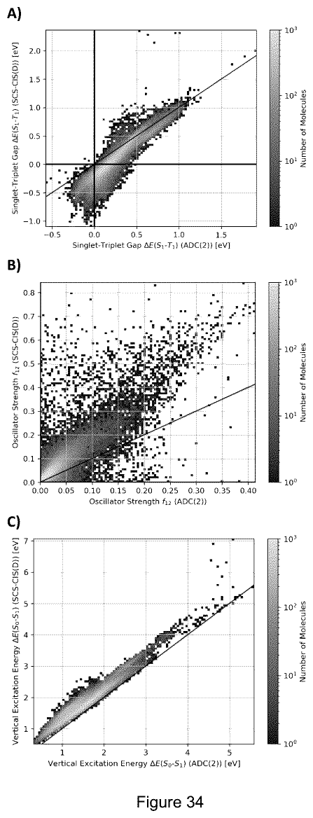

In addition to the respective property distributions for each of the levels of theory employed, the predictions obtained between all the methods used and ADC(2) were compared. FIGS. 26 to 34 depict the correlations between the excited state properties of interest against the corresponding properties at the ADC(2) level of theory.

Importantly, apart from SF-PBE50, all other methods reproduce the trends obtained at the ADC(2) level of theory very well as suggested by the reasonable linear correlations between the excited state properties. However, correlations between oscillator strengths generally seem to be somewhat lower which suggests it be more sensitive to the level of theory employed. Additionally, systematic discrepancies between the predicted properties at distinct levels of theory are relatively common. This is particularly noticeable from the comparison of the double-hybrid time-dependent density functional theory methods against ADC(2). For these methods, all three excited state properties of interested show systematic deviations. The linear correlation of the singlet-triplet gaps suggests a slope that would deviate significantly from 1 and an offset that would deviate significantly from 0. Consequently, they tend to estimate much more positive singlet-triplet gaps resulting in a very low number of compounds predicted to possess an inverted singlet-triplet gap. The opposite is observed for all the CIS(D)-based methods as the singlet-triplet gaps seem to be estimated systematically more negative compared to ADC(2). Thus, they also predict a significantly larger number of the compounds investigated to possess an inverted singlet-triplet gap. Closest agreement between ADC(2) and any other method is observed for CC2. Nevertheless, overall property trends are reproduced well by the majority of the methods employed.

Table summarizes the number of INVEST candidates and INVEST emitters based on the various levels of theory used in the course of this work. Therein, INVEST candidates refers to compounds with predicted negative singlet-triplet gaps. INVEST emitters refers to compounds with both predicted negative singlet-triplet gaps and predicted oscillator strengths larger than 0.01. Importantly, these results again illustrate the systematic offset that is observed in the singlet-triplet gaps between the various computational approaches used as shown above.

TABLE 5
Summary of virtual screening results at all the levels of theory considered in this work.
INVEST candidates refers to compounds with predicted negative singlet-triplet gaps. INVEST
emitters refers to INVEST candidates with predicted oscillator strength larger than 0.01.

| Level of Theory | INVEST Candidates | INVEST Emitters |
|---|---|---|
| ADC(2)/cc-pVDZ | 7643 (11%) | 1928 (3%) |
| SOS-ADC(2)/cc-pVDZ | 13880 (20%) | 5615 (8%) |
| RKS-ωB2PLYP′/def2- | 102 (0%) | 2 (0%) |
| SVP | | |
| UKS-ωB2PLYP/def2-SVP | 6 (0%) | 0 (0%) |
| SF-PBE50/def2-SVP | 50 (0%) | 10 (0%) |
| CC2/cc-pVDZ | 6828 (10%) | — |
| SOS-CC2/cc-pVDZ | 11085 (16%) | — |
| CIS(D)/cc-pVDZ | 34324 (50%) | 22501 (33%) |
| SOS-CIS(D)/cc-pVDZ | 32178 (46%) | 22254 (32%) |
| SCS-CIS(D)/cc-pVDZ | 32191 (47%) | 21890 (32%) |

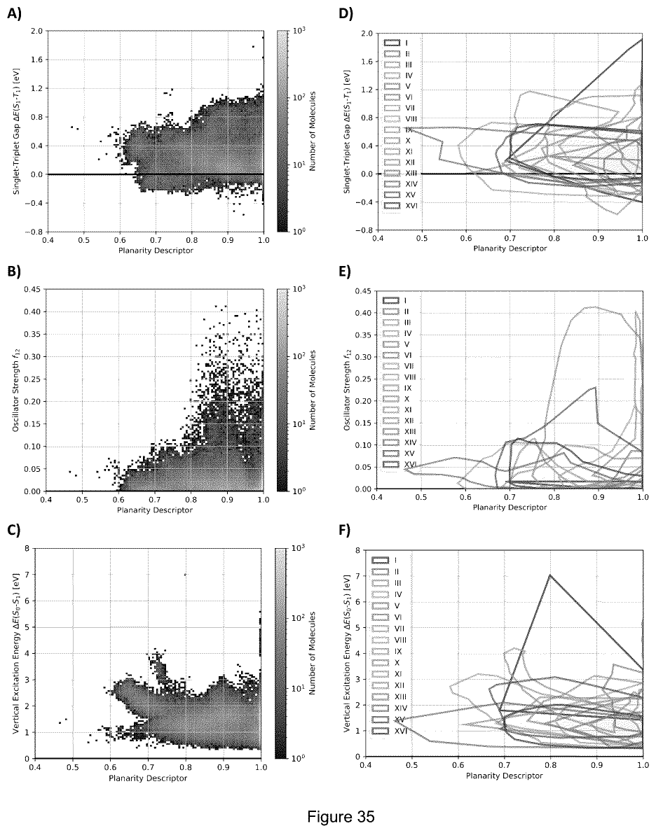

In addition to the results presented in the main text with respect to the planarity of the molecules, the two-dimensional property distribution densities of the planarity descriptor were also investigated together with the excited state properties of interest. The corresponding diagrams are provided in FIG. 35. As can be seen, there is no strong correlation between any of the properties with the planarity descriptor. This suggests that high planarity is not a necessary feature of organic INVEST emitters even though previous INVEST molecules were planar.

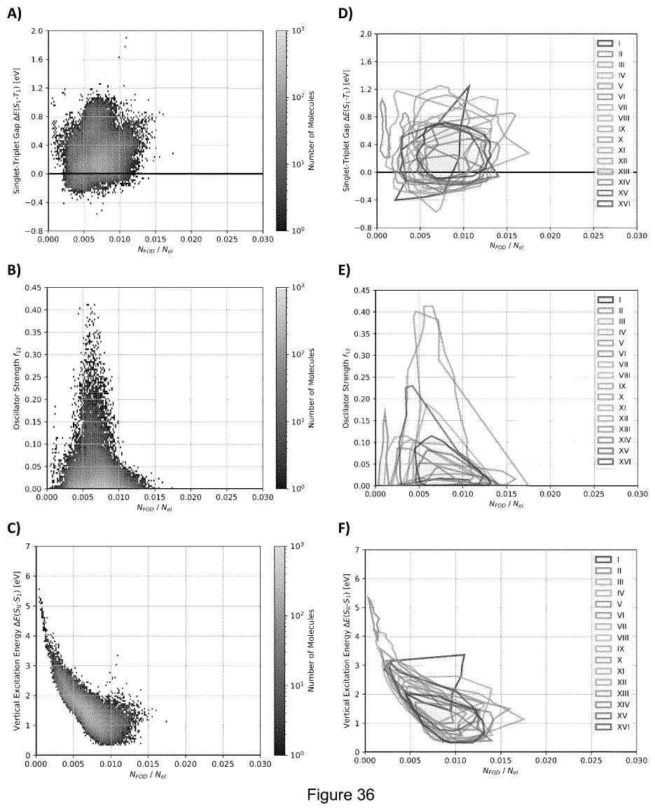

Validation. For the investigation of the integral of the fractional occupation number weighted electron density over all space normalized by the corresponding total number of electrons, the two-dimensional property distribution densities of the corresponding descriptor N_{FOD}/Nₑₗ were also inspected together with the excited state properties of interest. The corresponding plots are shown in FIG. 36. It only reveals a reasonable correlation between the vertical excitation energy and the normalized FOD descriptor. This correlation is not unexpected as smaller vertical excitation energies lead to a higher fractional electron occupation in the corresponding excited state under thermal excitation due to the reduced amount of energy necessary for the excitation as predicted by Fermi-Dirac statistics.

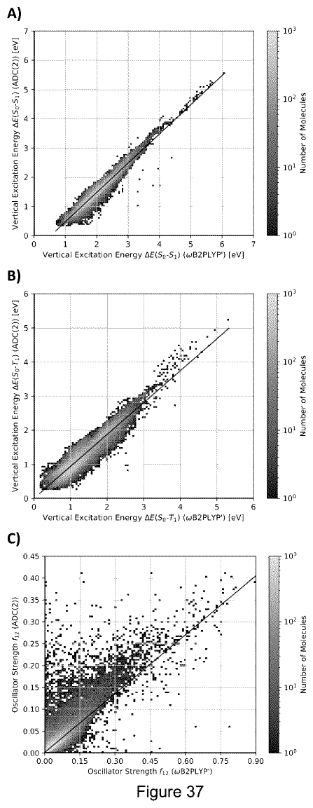

To be able to apply the excited state property correction for geometric relaxation to the corresponding properties at the ADC(2)/cc-pVDZ level of theory, the vertical excitation properties obtained from RKS-ωB2PLYP′/def2-SVP were calibrated in the ground state B3LYP/6-31G* geometries against the vertical excitation properties obtained from ADC(2)/cc-pVDZ in the ground state B97-3c geometries. Hence, robust linear regression was performed of the vertical excitation energy from S₀ to S₁, of the vertical excitation energy from S₀ to T₁ and of the oscillator strength using a Theil-Sen estimator^{375, 376} as implemented in the python package scikit-learn^{377,378} with a stochastic subpopulation of maximal size 25,000 in the random state 0. The corresponding regression results are summarized in Table 6 and illustrated in FIG. 37.

TABLE 6
Calibration results of excited state properties at the ωB2PLYP′ level of theory against
the corresponding properties at the ADC(2) level of theory. Calibration was performed using a
robust linear Theil-Sen estimator according to the equation: Property(ADC(2)) = a .
Property(ωB2PLYP′) + b.

| Property | a | b | R² |
|---|---|---|---|
| ΔE(S₀-S₁) [eV] | 1.001 | −0.533 | 0.95 |
| ΔE(S₀-T₁) [eV] | 0.936 | −0.003 | 0.90 |
| f₁₂ | 0.449 | 0.001 | 0.71 |

Calibrated singlet-triplet gaps were obtained as the difference between the calibrated vertical excitation energies from S₀ to S₁ and T₁, respectively. These calibrations were applied to both the vertical and relaxed excited state properties at the RKS-ωB2PLYP′/def2-SVP level of theory. Subsequently, the difference between the calibrated relaxed and vertical excited state properties was taken as excited state property correction due to geometry relaxation. Importantly, when the property corrections led to a negative vertical excitation energy between S₀ and S₁, the corresponding corrected property was arbitrarily set to 0 as no such state order inversion was observed at the original RKS-ωB2PLYP′/def2-SVP level of theory and it is likely an artifact of the procedure adopted for correction rather than physically meaningful. Similarly, when the property corrections led to a negative oscillator strength for the transition between S₀ and S₁, the corresponding corrected property was also arbitrarily set to 0. Notably, this only had to be done for a relatively small number of compounds.

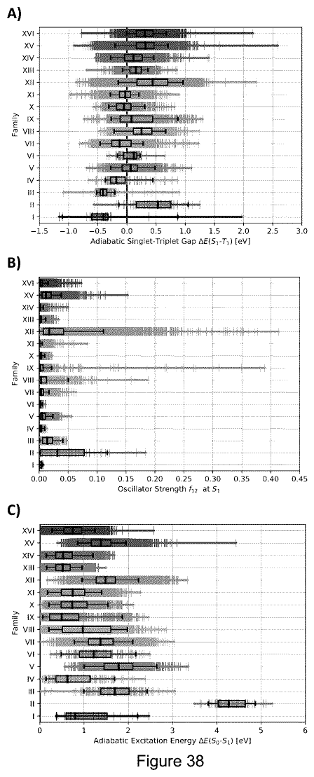

After the correction was applied, the obtained corrected excited state properties were inspected thoroughly. First, the property ranges and distributions were divided into the 16 compound families and studied, which are shown in FIG. 38. The results show that the singlet-triplet gap distributions are shifted significantly towards more negative values. The oscillator gap distributions do not change significantly, and the vertical excitation energy distributions are shifted towards smaller values.

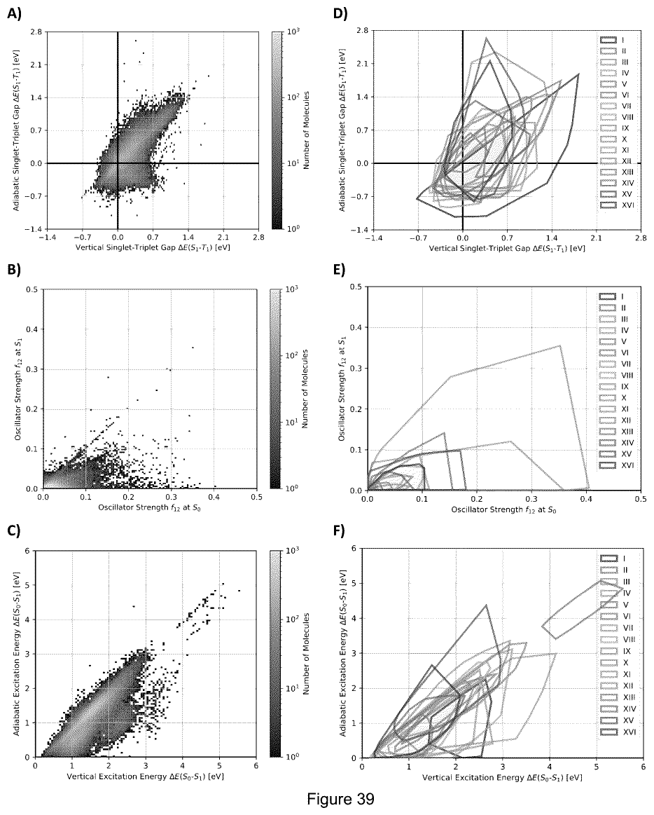

This was also observed when the corresponding vertical and corrected, i.e., relaxed, excited state properties at the ADC(2) level of theory are directly compared in FIG. 39. Comparing adiabatic and vertical singlet-triplet gaps it was shown that while the largest fraction of the compounds seems to have comparable values for both properties, the fraction of molecules with more negative adiabatic singlet-triplet gaps is larger than the fraction of molecules with more positive ones relative to the vertical property. For the corrected and uncorrected oscillator strength, it is shown that they tend not to compare very favorable. Nevertheless, for most compounds, both corrected and uncorrected oscillator strengths are very small. When comparing the vertical and adiabatic excitation energies, it was seen that the adiabatic values tend to be smaller which is to be expected as the geometry relaxation in S₁ is necessarily a downhill process and S₀ in the S₁ geometry is higher in energy than S₀ in the S₀ geometry. Nevertheless, the corresponding differences tend to be comparably small.

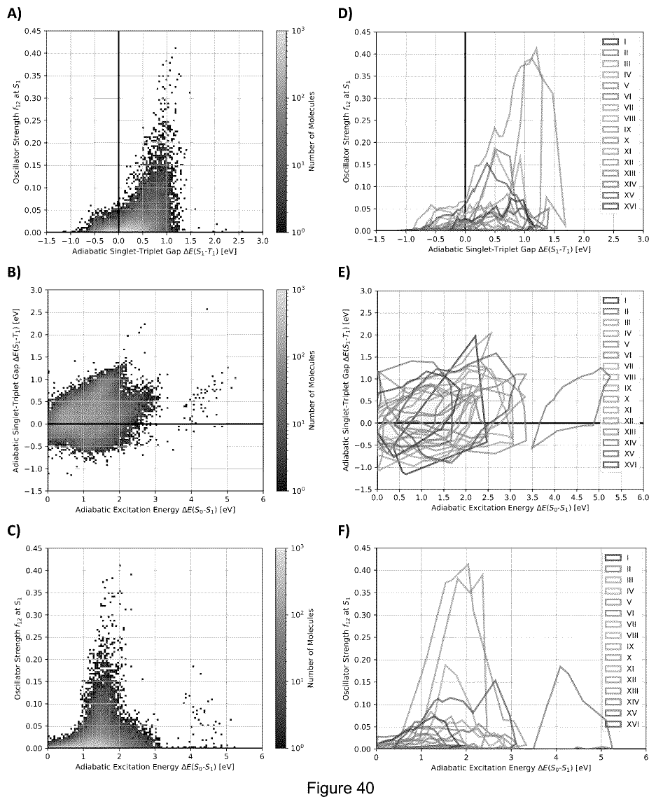

The two-dimensional property distribution densities of pairs of adiabatic excited state properties were studied. They are shown in FIG. 40. Overall, they show that the general trends and correlations have not changed between the vertical and corrected excited state properties. As mentioned previously, they illustrate again the shift of the singlet-triplet gap distribution towards more negative values and the shift of excitation energies towards smaller values. Notably, the trade-off between singlet-triplet gap and oscillator strength is still very pronounced.

Finally, the evaluation of the number of molecules was repeated with favorable predicted excited state properties in each of the structural families investigated. Table 1 shows the statistics on predicted INVEST candidates, i.e., compounds with predicted negative singlet-triplet gaps, and INVEST emitters, i.e., compounds with predicted negative singlet-triplet gaps and predicted oscillator strengths larger than 0.01, at the ADC(2) level of theory including property corrections due to excited state geometry relaxation. As can be seen, compared to the properties estimated based on vertical excited state transitions (cf. Table 1), the numbers and fractions of INVEST candidates are increased significantly. In particular, the number of 5 predicted INVEST candidates is increased for every single molecular family considered. In contrast, the numbers of predicted INVEST emitters is only slightly increased in total. Looking at the changes for the structural classes considered, the number increases for some families but decreases for others, and a very large increase is only observed for cycloocta-1,3,5,7-tetraene (family III).

TABLE 7
Molecular families considered and summary of virtual screening results at the
ADC(2)/cc-pVDZ level of theory including property corrections due to geometry relaxation.
INVEST candidates refers to compounds with predicted negative singlet-triplet gaps. INVEST
emitters refers to INVEST candidates with predicted oscillator strength larger than 0.01.

| | | Parent | | | |
|---|---|---|---|---|---|
| | | Ring | | INVEST | INVEST |
| Family | Parent Compound Name | Size | Compounds | Candidates | Emitters |
| I | Cyclobuta-1,3-diene | 4 | 21 | 17 (81%) | 0 (0%) |
| II | Benzene | 6 | 56 | 7 (13%) | 1 (2%) |
| III | Cycloocta-1,3,5,7-tetraene | 8 | 336 | 328 (98%) | 210 (63%) |
| IV | Pentalene | 8 | 248 | 191 (77%) | 12 (5%) |
| V | Azulene | 10 | 1493 | 552 (37%) | 28 (2%) |
| VI | Bowtiene | 10 | 126 | 34 (27%) | 0 (0%) |
| VII | Heptalene | 12 | 8513 | 5597 (66%) | 802 (9%) |
| VIII | Zurlene | 12 | 2591 | 430 (17%) | 51 (2%) |
| IX | s-Indacene | 12 | 1784 | 691 (39%) | 39 (2%) |
| X | as-Indacene | 12 | 1296 | 783 (60%) | 21 (2%) |
| XI | Anthrazulene | 14 | 10709 | 6144 (57%) | 12 (0%) |
| XII | Phenazulene | 14 | 15551 | 1922 (12%) | 640 (4%) |
| XIII | Dicyclohepta[a,c]cyclobutene | 14 | 3996 | 552 (14%) | 0 (0%) |
| XIV | Dicyclopenta[a,e]cyclooctene | 14 | 3996 | 1261 (32%) | 12 (0%) |
| XV | Dicyclopenta[a,d]cyclooctene | 14 | 10709 | 1311 (12%) | 260 (2%) |
| XVI | Dicyclopenta[a,c]cyclooctene | 14 | 7776 | 408 (5%) | 45 (1%) |
| I-XVI | — | — | 69201 | 20228 (29%) | 2133 (3%) |

### Supplementary Discussion

Virtual Screening. Similar to previous computational studies on azaphenalenes,³⁷⁹ systematic deviations were observed between the excited state properties obtained using double-hybrid time-dependent density functional theory and correlated excited state methods like ADC(2), CC2 and CIS(D). Consequently, the former lead to significantly more positive predictions. This offset is in part caused by a systematic overestimation of the vertical excitation energies between the S₀ and S₁ states. However, trends between these methods are reproduced well making the use of the more affordable density functional approximations suitable methods for extensive virtual screening. However, the good performance of CIS(D)-based methods suggests that more reliable density functional approximations could be proposed and this was indeed demonstrated in a recent study that was published during the course of this work.³⁸⁰ Hence, further improvements that are currently being made to the toolbox for the simulation excited state properties of potential INVEST emitters suggest that better property predictions will be available at a lower computational cost in the near future.

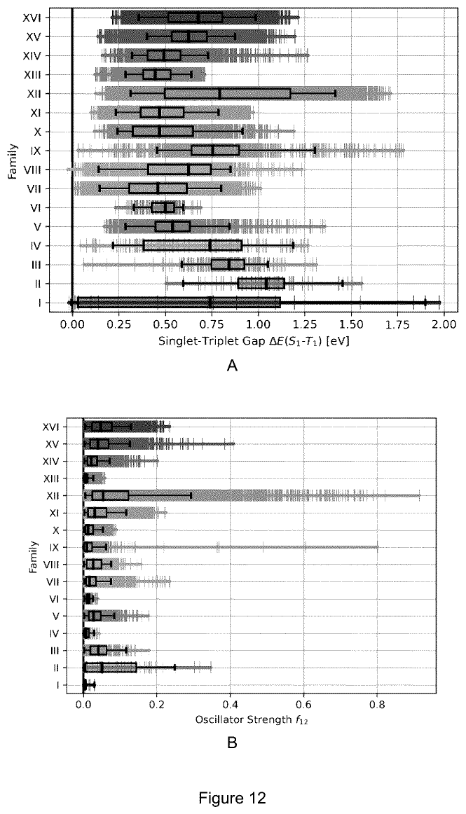

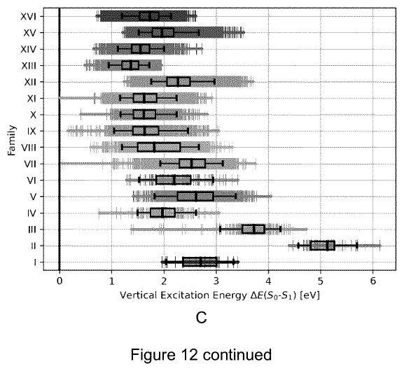

Validation. Since the correlation of simulated oscillator strength values tends to be significantly lower between distinct levels of theory compared to state energy differences, the corresponding correction due to geometric relaxation is likely the least reliable. This is reflected in the comparably small coefficient of determination for the calibration of properties at the ωB2PLYP′/def2-SVP and ADC(2)/cc-pVDZ levels of theory in FIG. 12C. Thus, it is not clear whether the poor correlation between the oscillator strengths from vertical excitations and oscillator strengths corrected for geometric relaxation is meaningful and signifies the importance of using relaxed excited state geometries for their simulation or whether it is an artifact of the methodology employed for the correction. Further investigations in that regard are necessary. However, for both vertical excitation energies and singlet-triplet gaps, we observe reasonable correlations showing that the vertical state energy differences are likely a reasonable approximation of the corresponding adiabatic state energy differences.

### Example 5 Exemplary Compounds of Families I, IV, VI, VIII, X, XI, XIII, XIV, XV, and XVI

Some exemplary compounds of Family I are shown below:

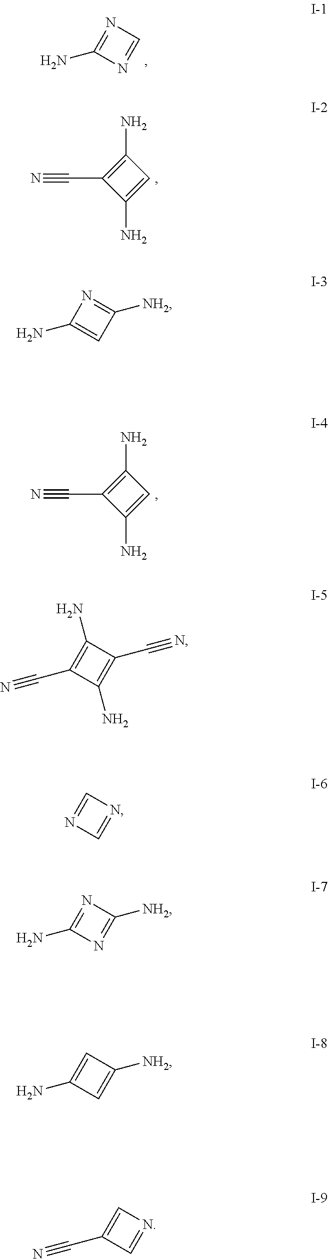

5 Some exemplary compounds of Family IV are shown below:

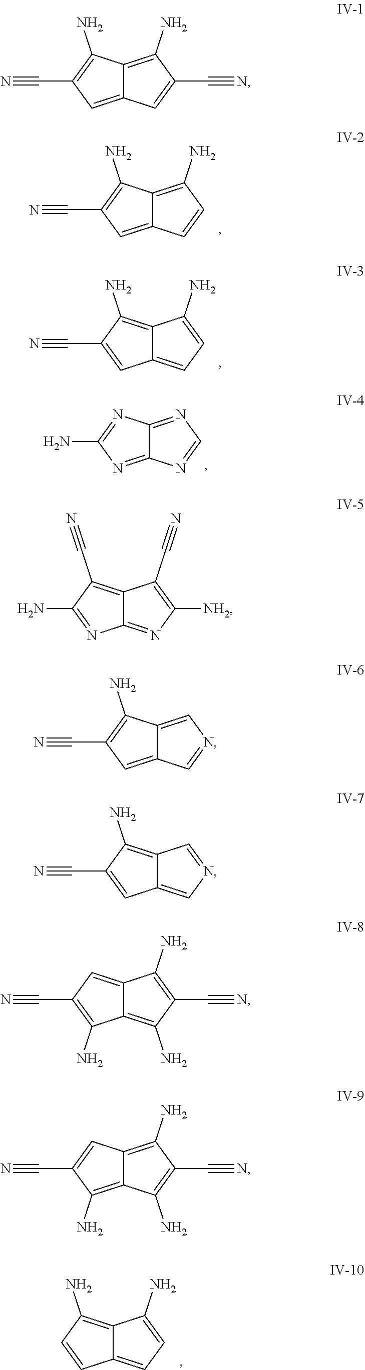

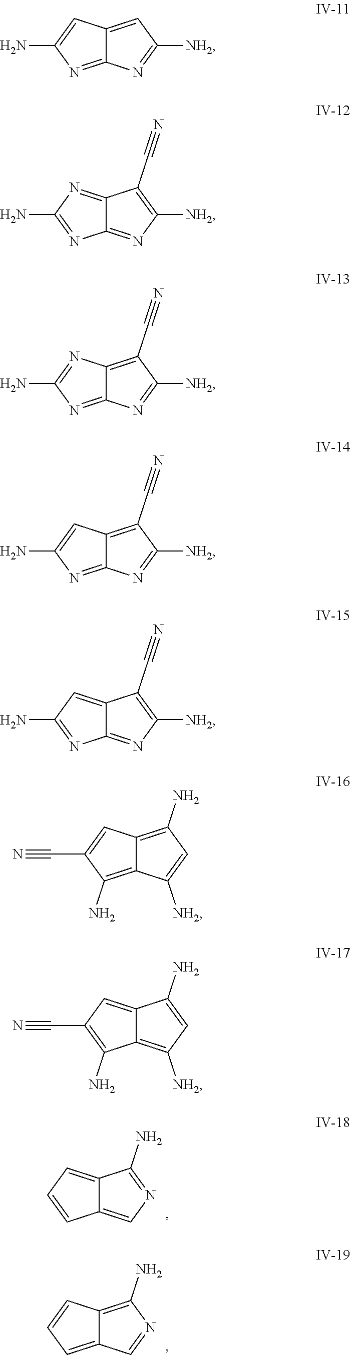

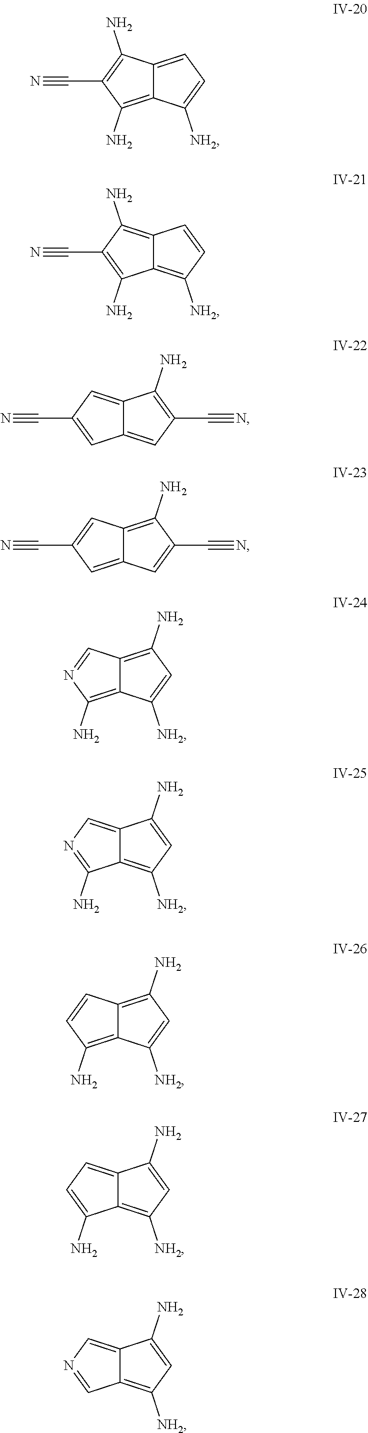

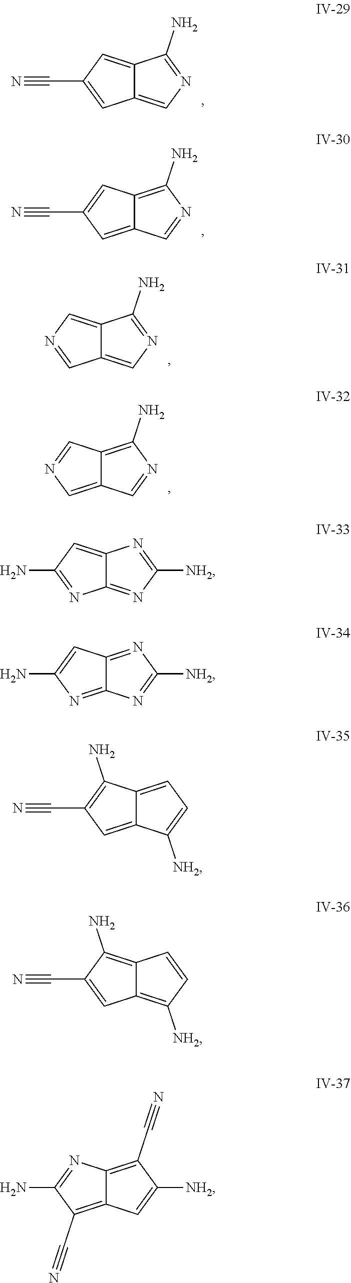

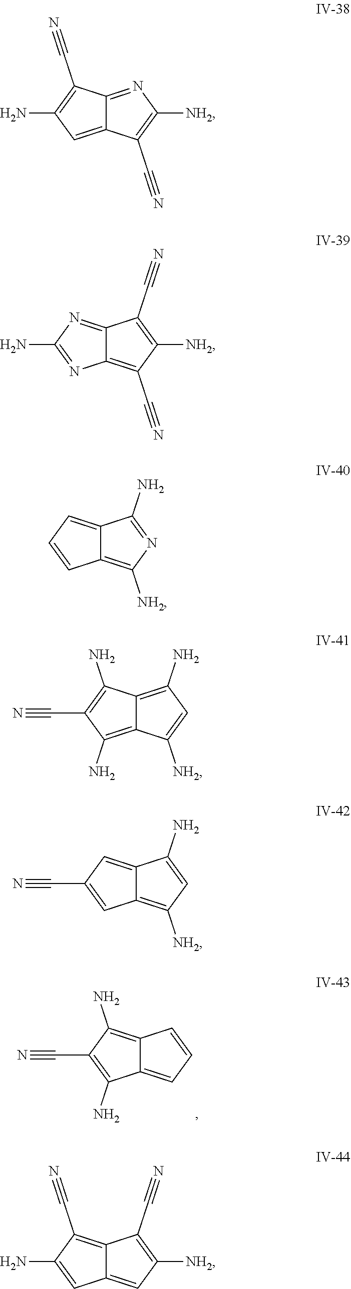

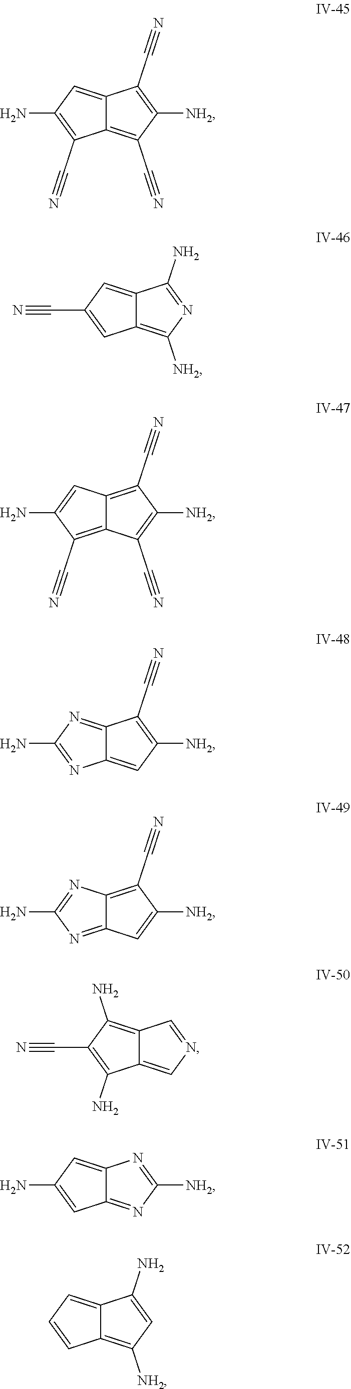

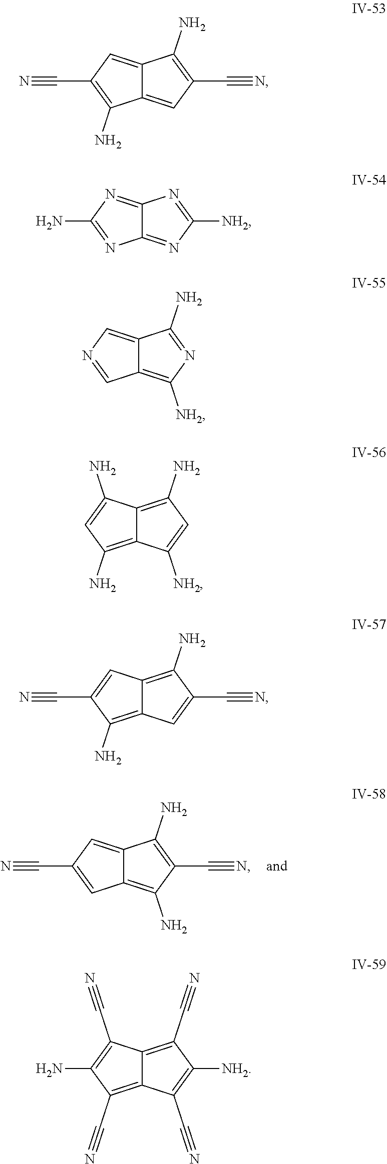

Some exemplary compounds of Family VI are shown below:

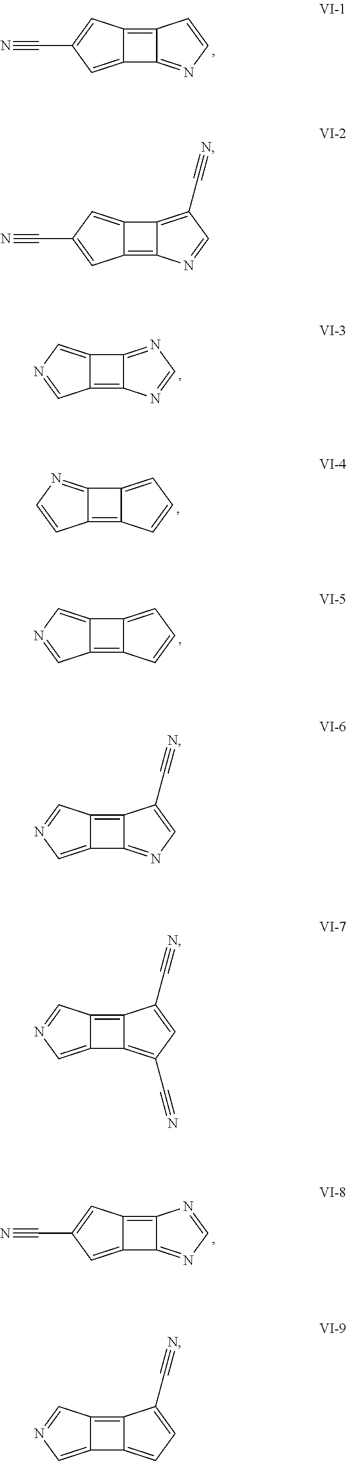

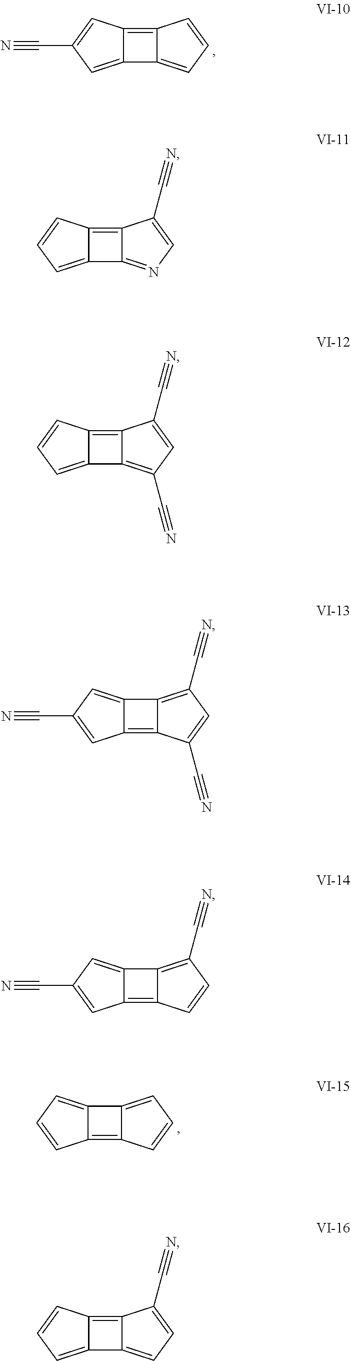

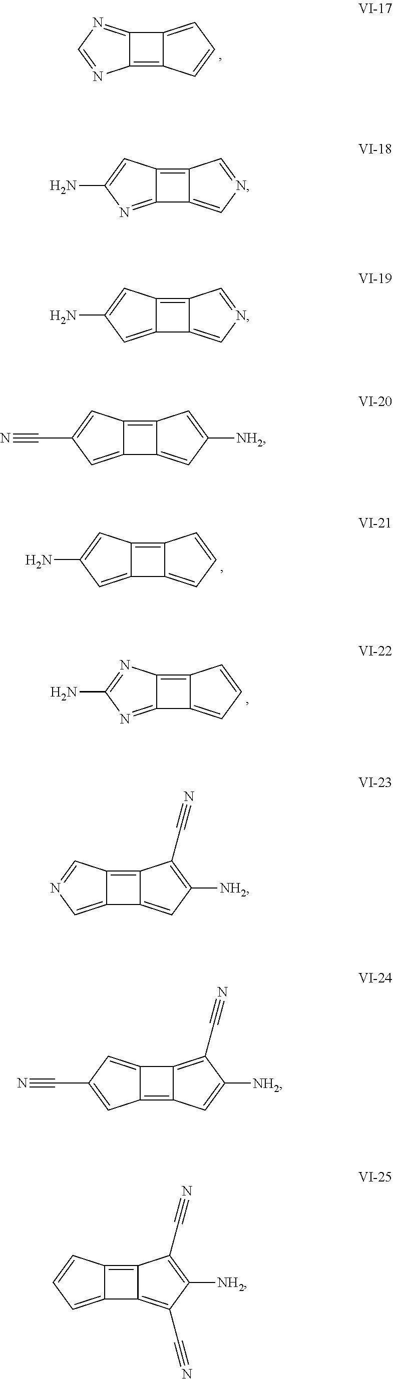

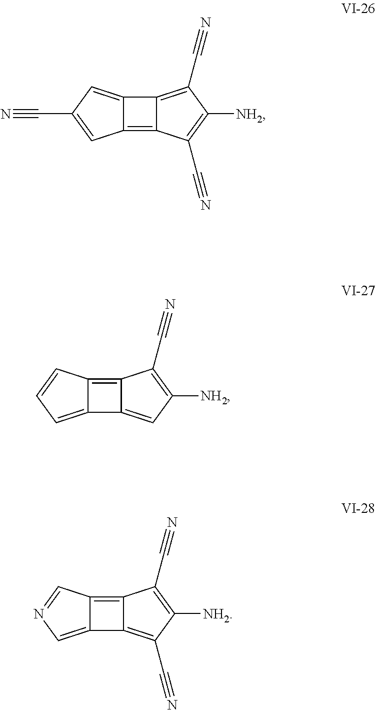 [SMILES: N#Cc1cc2c3c(C#N)c(N)c(C#N)c-3c-2c1 ; N#Cc1c(N)cc2c3cccc-3c1-2 ; N#Cc1c(N)c(C#N)c2c3cncc-3c1-2]

Some exemplary compounds of Family VIII are shown below:

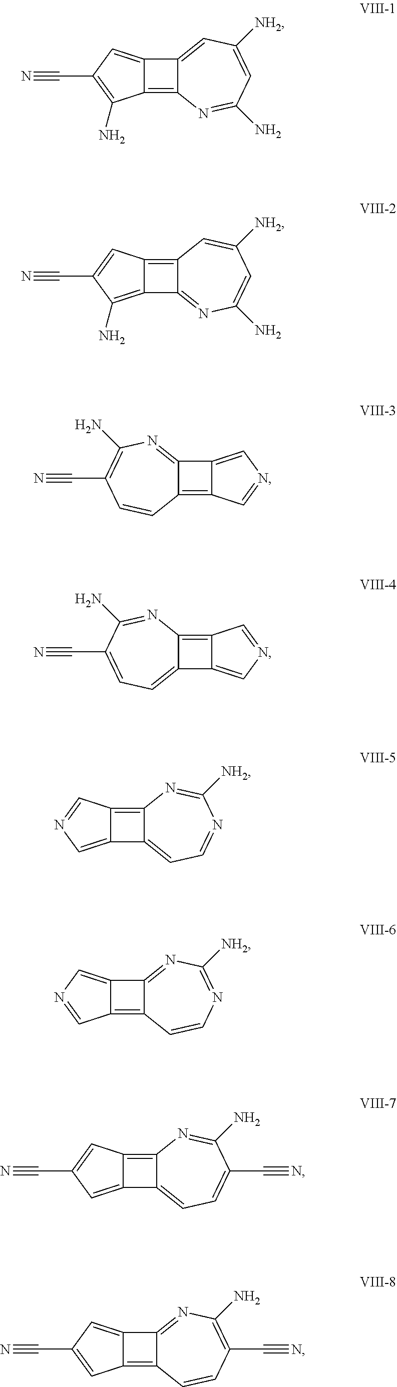

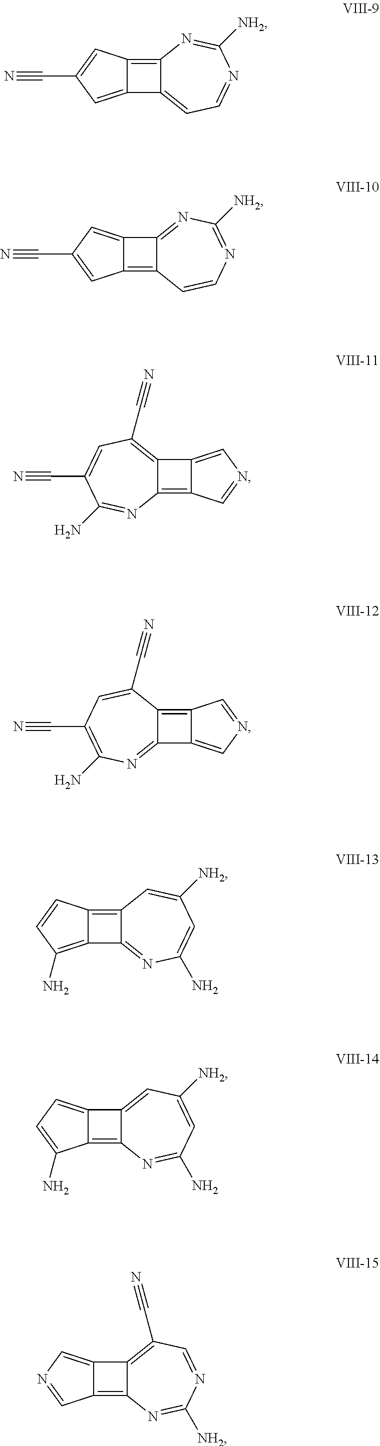

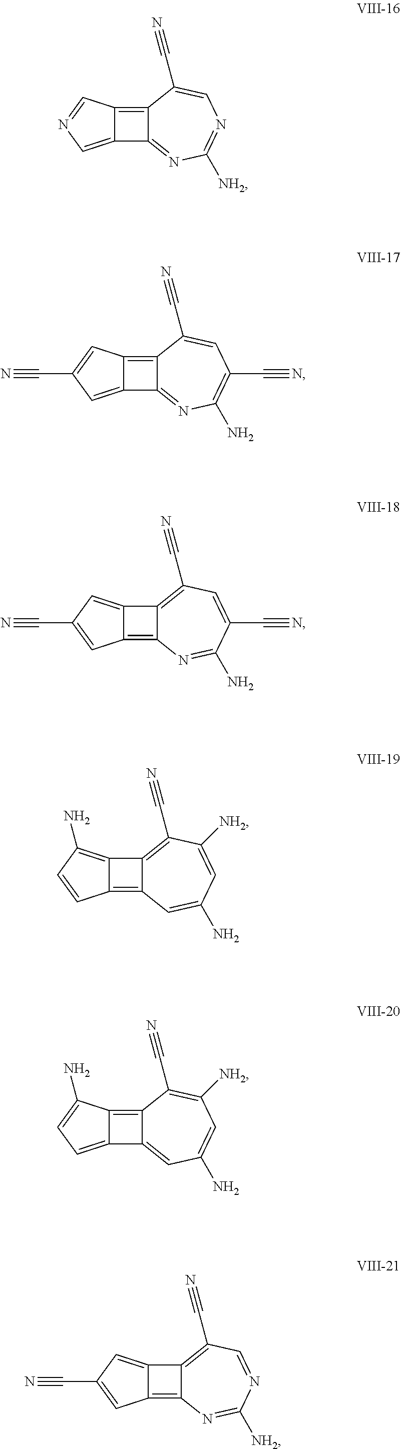 [SMILES: N#CC1=CN=C(N)N=C2C3=CN=CC3=C12 ; N#CC1=CC2=C3C(C#N)=CC(C#N)=C(N)N=C3C2=C1 ; N#CC1=CC2=C3N=C(N)C(C#N)=CC(C#N)=C3C2=C1 ; N#CC1=C2C(=C3C=CC(N)=C32)C=C(N)C=C1N ; N#CC1=C(N)C=C(N)C=C2C3=CC=C(N)C3=C21 ; N#CC1=CC2=C3N=C(N)N=CC(C#N)=C3C2=C1]

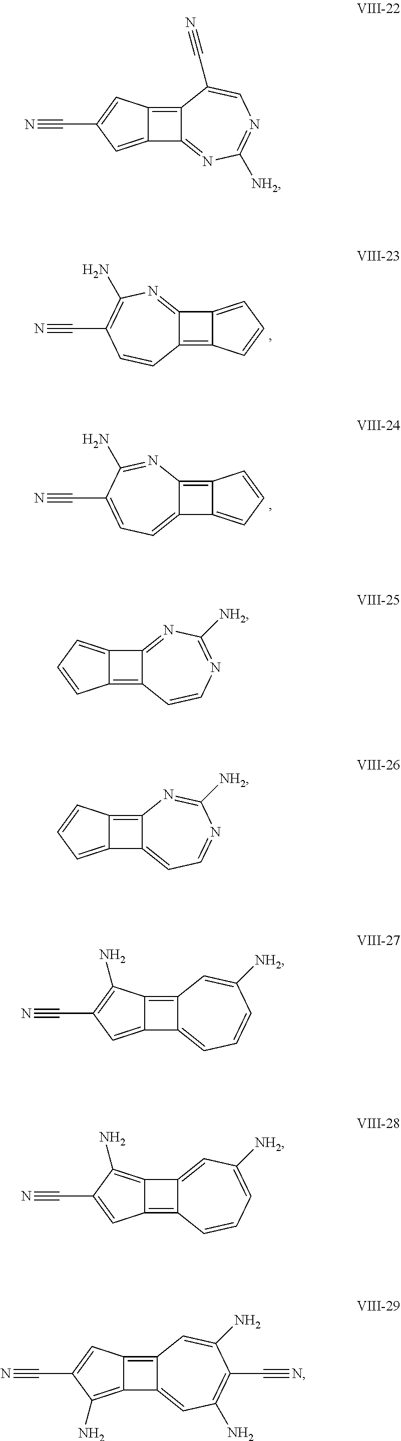

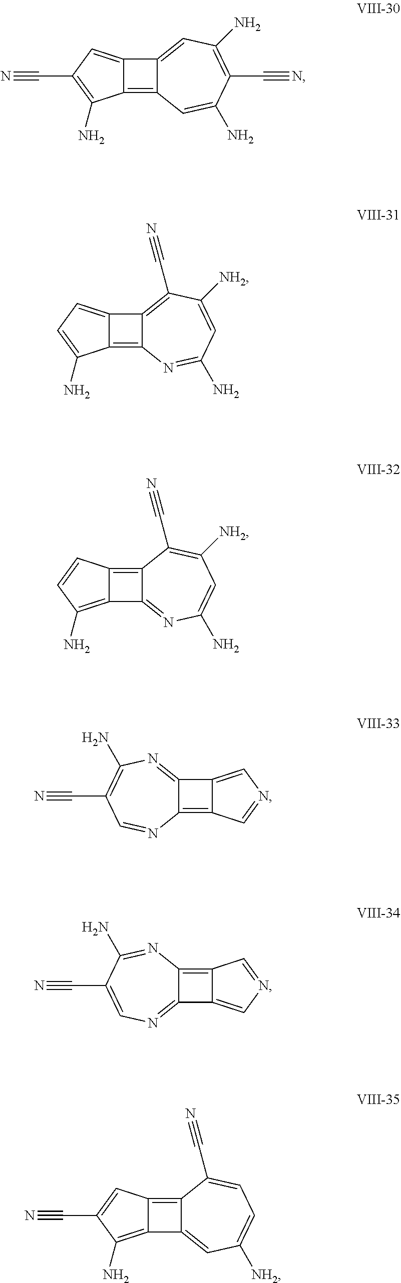 [SMILES: N#CC1=C(N)C2=C3C=C(N)C(C#N)=C(N)C=C3C2=C1 ; N#CC1=C2C3=CC=C(N)C3=C2N=C(N)C=C1N ; N#CC1=C(N)C=C(N)N=C2C3=C(N)C=CC3=C21 ; N#CC1=C(N)N=C2C3=CN=CC3=C2N=C1 ; N#CC1=CN=C2C3=CN=CC3=C2N=C1N ; N#CC1=CC2=C3C(C#N)=CC=C(N)C=C3C2=C1N]

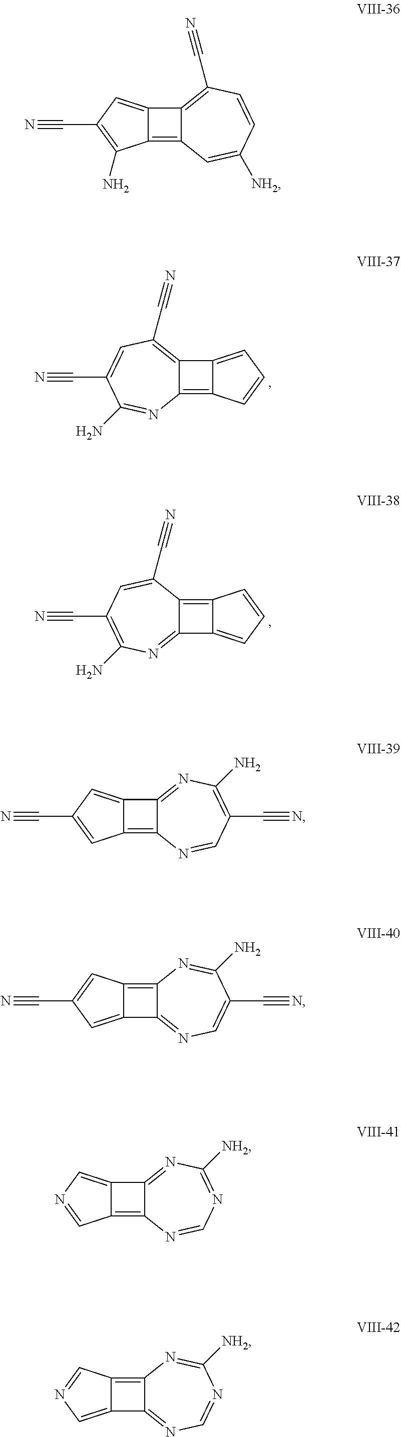

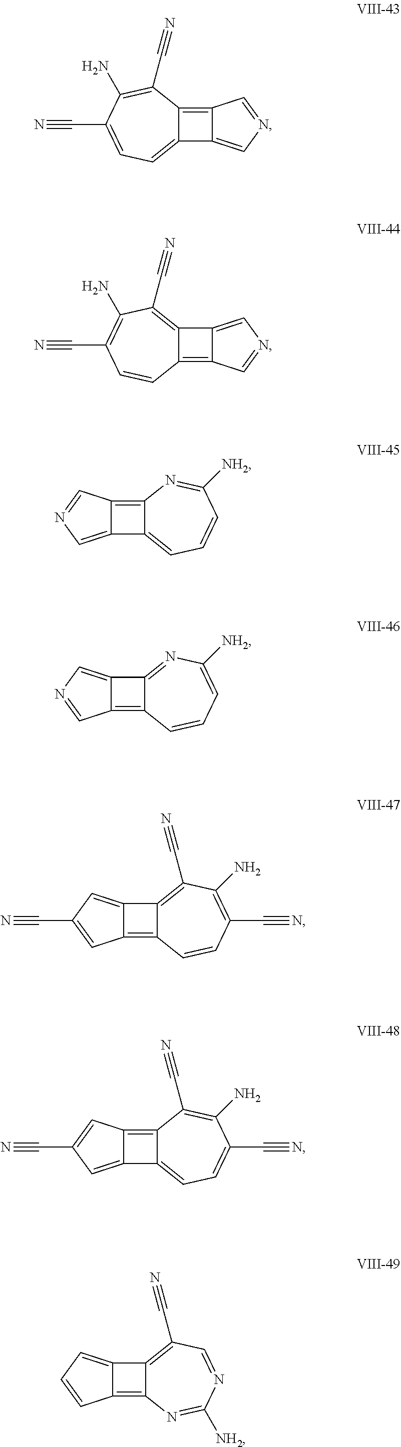

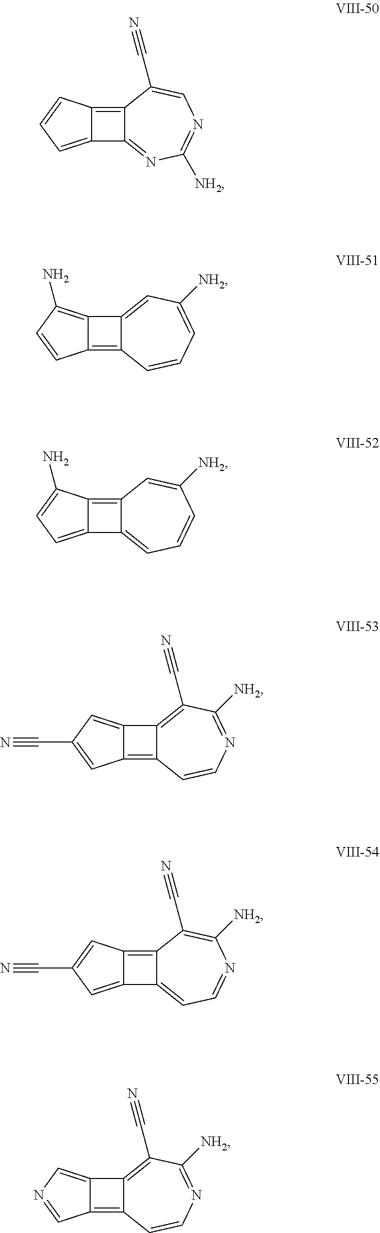 [SMILES: N#CC1=CN=C(N)N=C2C3=CC=CC3=C12 ; NC1=CC=CC2=C3C=CC(N)=C3C2=C1 ; NC1=CC2=C3C(N)=CC=C3C2=CC=C1 ; N#CC1=CC2=C3C=CN=C(N)C(C#N)=C3C2=C1 ; N#CC1=CC2=C3C(=CC=NC(N)=C3C#N)C2=C1 ; N#CC1=C2C3=CN=CC3=C2C=CN=C1N]

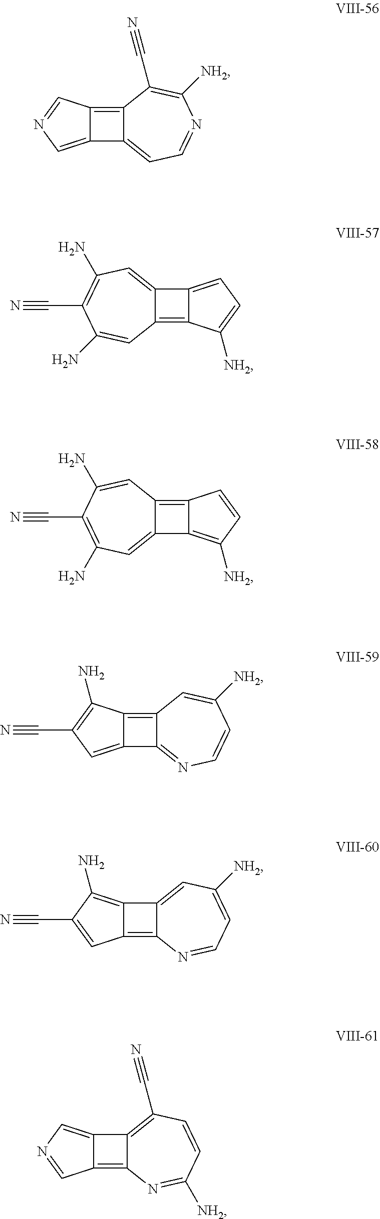 [SMILES: N#CC1=C(N)N=CC=C2C3=CN=CC3=C21 ; N#CC1=C(N)C=C2C3=CC=C(N)C3=C2C=C1N ; N#CC1=C(N)C=C2C(=C3C=CC(N)=C23)C=C1N ; N#CC1=C(N)C2=C3C=C(N)C=CN=C3C2=C1 ; N#CC1=CC2=C3N=CC=C(N)C=C3C2=C1N ; N#CC1=C2C3=CN=CC3=C2N=C(N)C=C1]

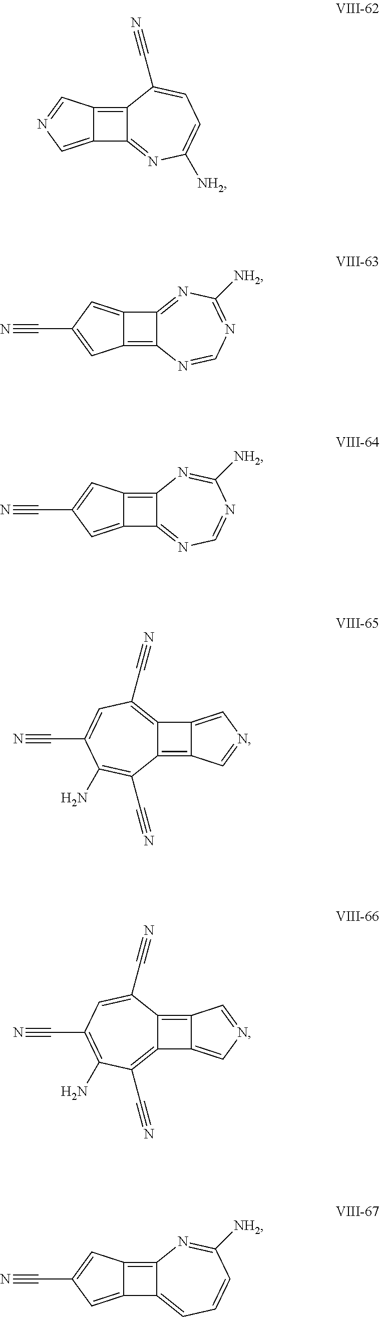 [SMILES: N#CC1=CC=C(N)N=C2C3=CN=CC3=C12 ; N#CC1=CC2=C3N=CN=C(N)N=C3C2=C1 ; N#CC1=CC2=C3N=C(N)N=CN=C3C2=C1 ; N#CC1=CC(C#N)=C2C3=CN=CC3=C2C(C#N)=C1N ; N#CC1=CC(C#N)=C(N)C(C#N)=C2C3=CN=CC3=C12 ; N#CC1=CC2=C3N=C(N)C=CC=C3C2=C1]

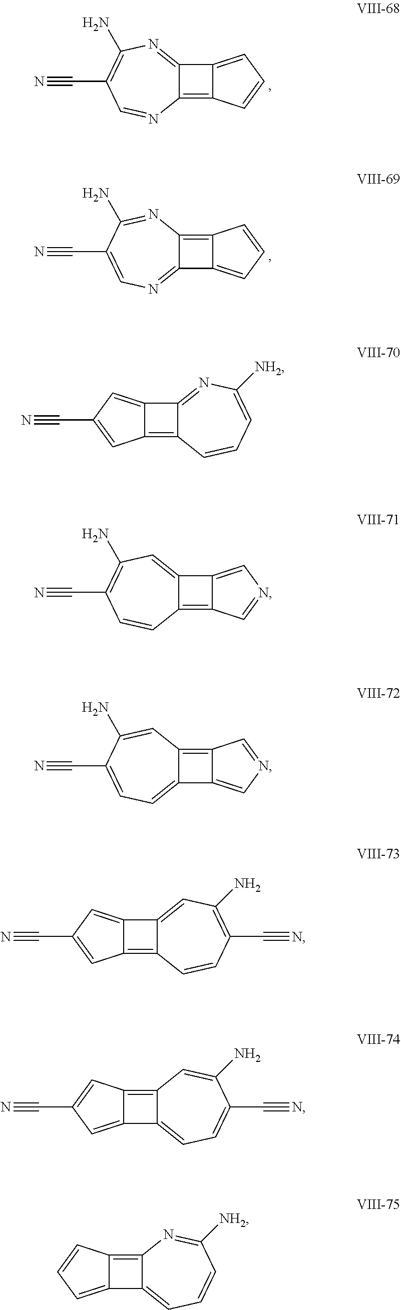

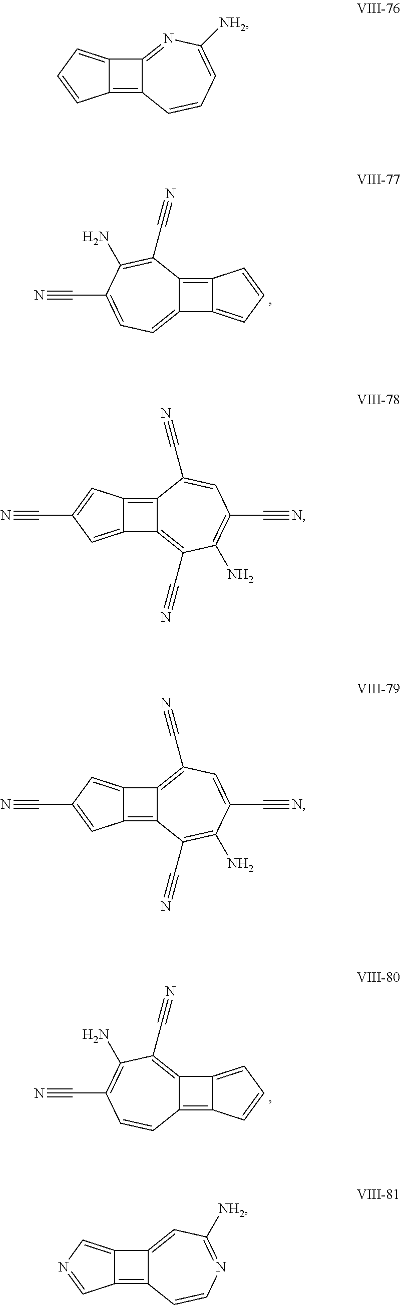 [SMILES: NC1=CC=CC2=C3C=CC=C3C2=N1 ; N#CC1=CC=C2C3=CC=CC3=C2C(C#N)=C1N ; N#CC1=CC2=C3C(C#N)=CC(C#N)=C(N)C(C#N)=C3C2=C1 ; N#CC1=CC2=C3C(C#N)=C(N)C(C#N)=CC(C#N)=C3C2=C1 ; N#CC1=C(N)C(C#N)=C2C3=CC=CC3=C2C=C1 ; NC1=NC=CC2=C3C=NC=C3C2=C1]

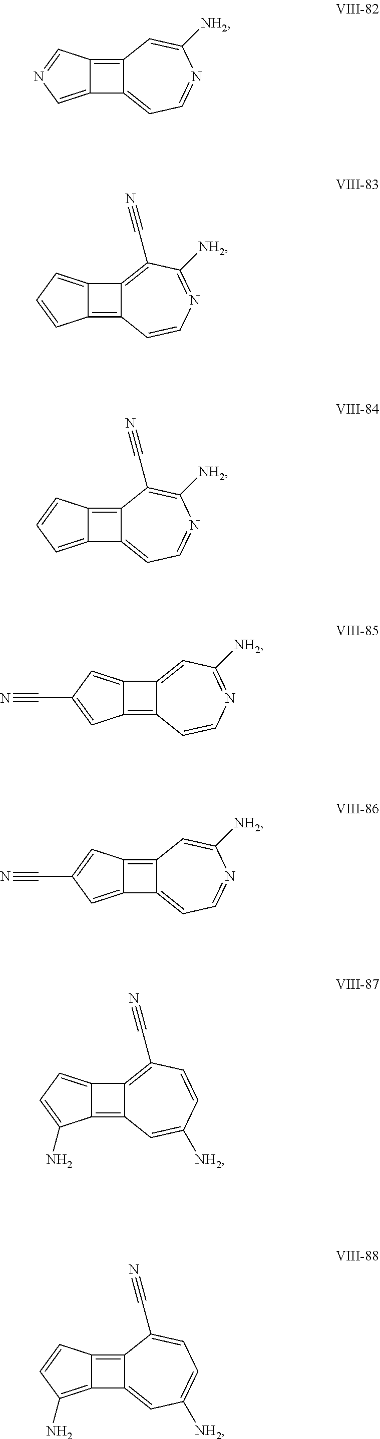

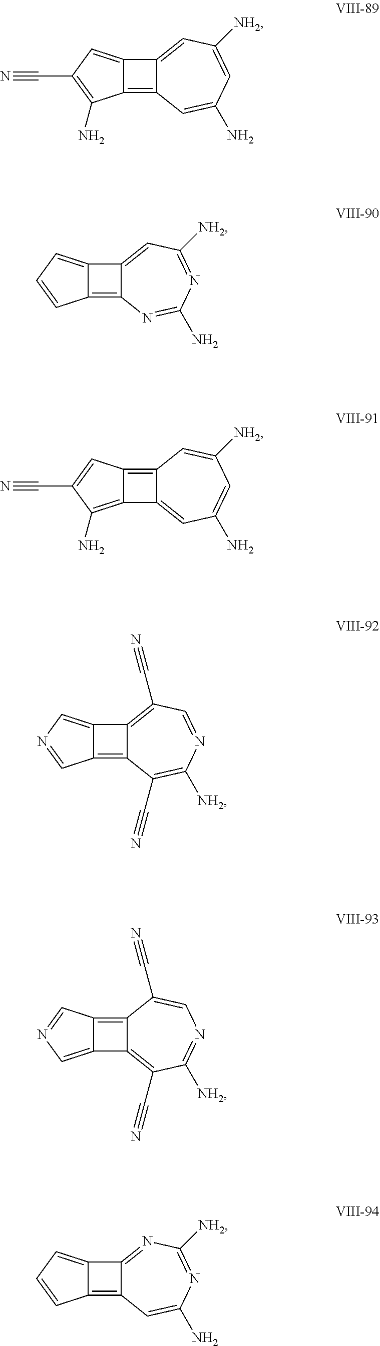 [SMILES: N#CC1=C(N)C2=C3C=C(N)C=C(N)C=C3C2=C1 ; NC1=NC(N)=NC2=C3C=CC=C3C2=C1 ; N#CC1=CC2=C3C=C(N)C=C(N)C=C3C2=C1N ; N#CC1=C2C3=CN=CC3=C2C(C#N)=C(N)N=C1 ; N#CC1=CN=C(N)C(C#N)=C2C3=CN=CC3=C12 ; NC1=CC2=C3C=CC=C3C2=NC(N)=N1]

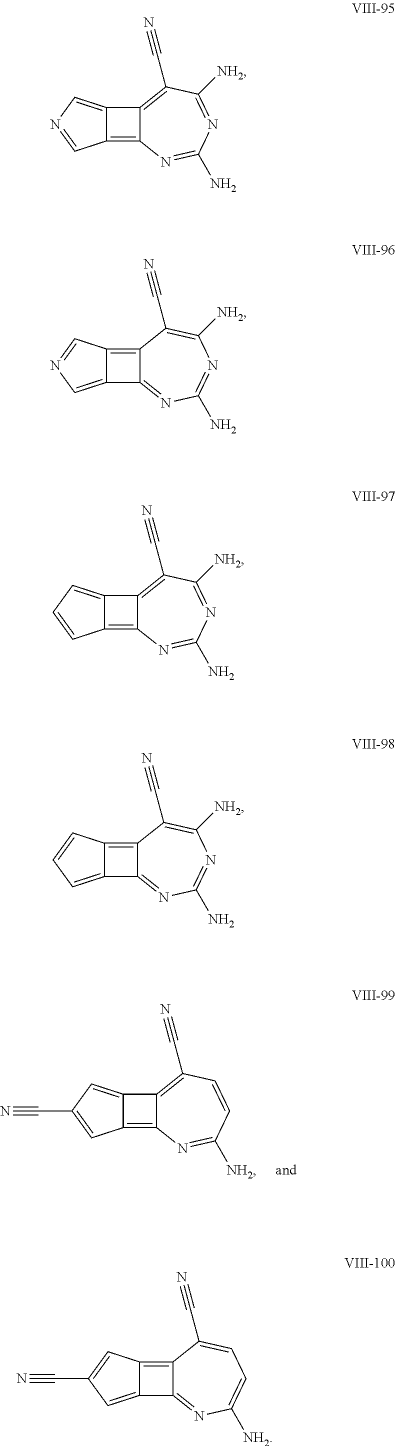

Some exemplary compounds of Family X are shown below:

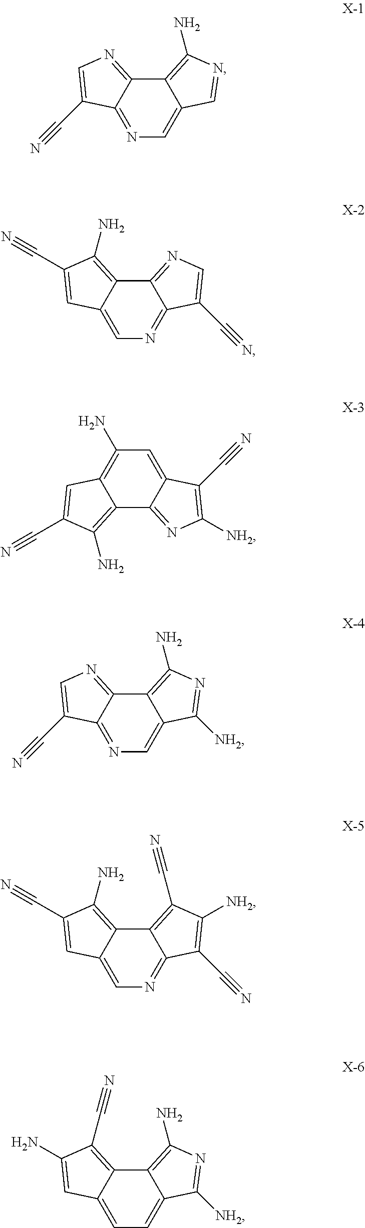 [SMILES: N#CC1=CN=c2c1ncc1c2=C(N)N=C1 ; N#CC1=Cc2cnc3c(c2=C1N)=NC=C3C#N ; N#CC1=Cc2c(N)cc3c(c2=C1N)=NC(N)=C3C#N ; N#CC1=CN=c2c1ncc1c2=C(N)N=C1N ; N#CC1=Cc2cnc3c(c2=C1N)=C(C#N)C(N)=C3C#N ; N#CC1=c2c(ccc3c2=C(N)N=C3N)C=C1N]

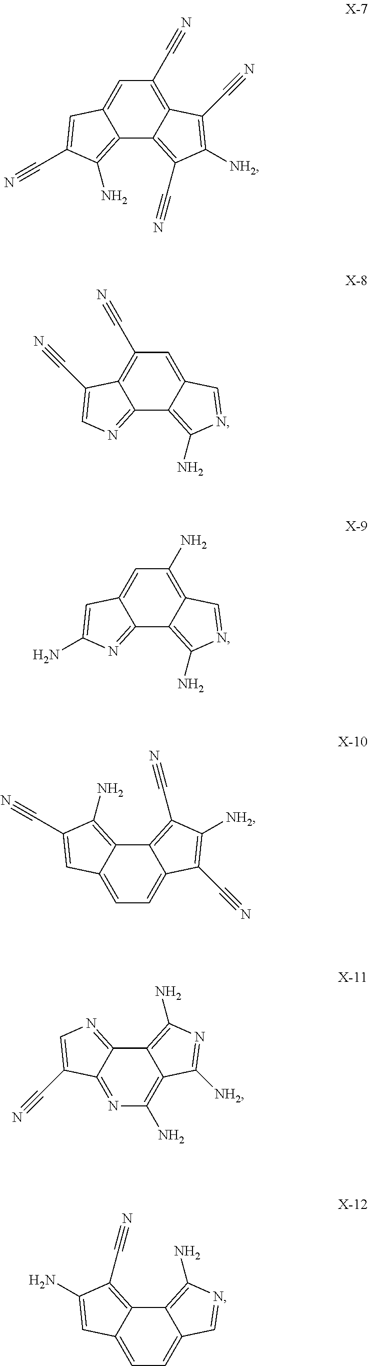 [SMILES: N#CC1=Cc2cc(C#N)c3c(c2=C1N)=C(C#N)C(N)=C3C#N ; N#CC1=CN=c2c1c(C#N)cc1c2=C(N)N=C1 ; NC1=Cc2cc(N)c3c(c2=N1)=C(N)N=C3 ; N#CC1=Cc2ccc3c(c2=C1N)=C(C#N)C(N)=C3C#N ; N#CC1=CN=c2c1nc(N)c1c2=C(N)N=C1N ; N#CC1=c2c(ccc3c2=C(N)N=C3)C=C1N]

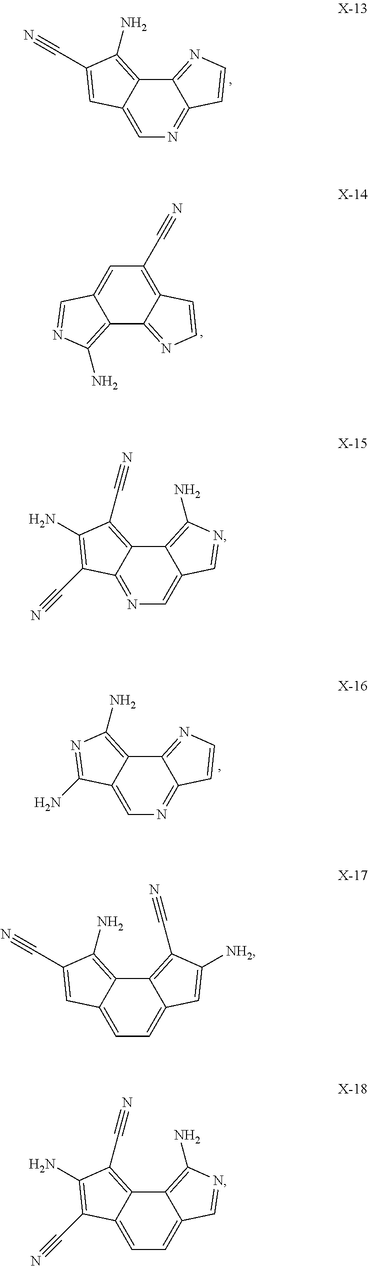 [SMILES: N#CC1=Cc2cnc3c(c2=C1N)=NC=C3 ; N#Cc1cc2c(c3c1C=CN=3)=C(N)N=C2 ; N#CC1=C(N)C(C#N)=c2c1ncc1c2=C(N)N=C1 ; NC1=NC(N)=c2c1cnc1c2=NC=C1 ; N#CC1=Cc2ccc3c(c2=C1N)=C(C#N)C(N)=C3 ; N#CC1=C(N)C(C#N)=c2c1ccc1c2=C(N)N=C1]

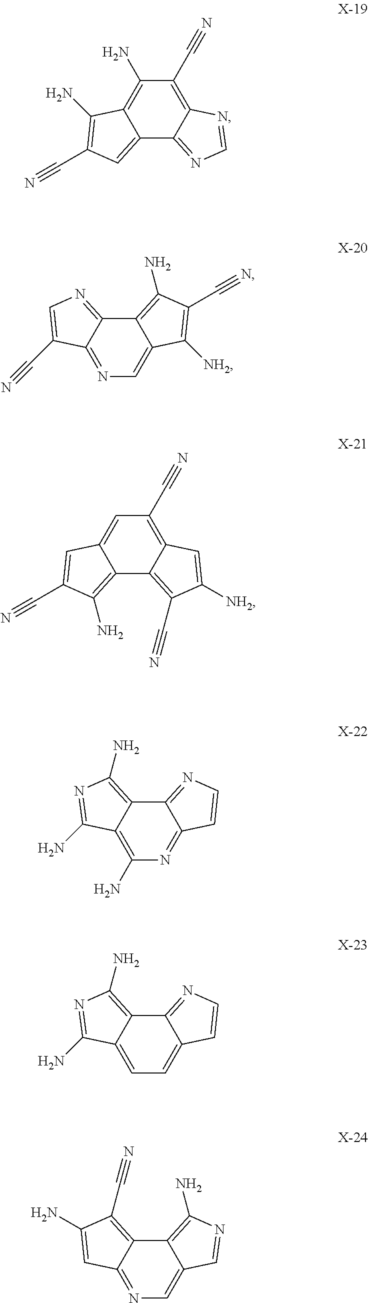 [SMILES: N#CC1=C(N)c2c(N)c(C#N)c3c(c2=C1)=NC=N3 ; N#CC1=CN=c2c1ncc1c2=C(N)C(C#N)=C1N ; N#CC1=Cc2cc(C#N)c3c(c2=C1N)=C(C#N)C(N)=C3 ; NC1=NC(N)=c2c1c(N)nc1c2=NC=C1 ; NC1=NC(N)=c2c1ccc1c2=NC=C1 ; N#CC1=c2c(ncc3c2=C(N)N=C3)C=C1N]

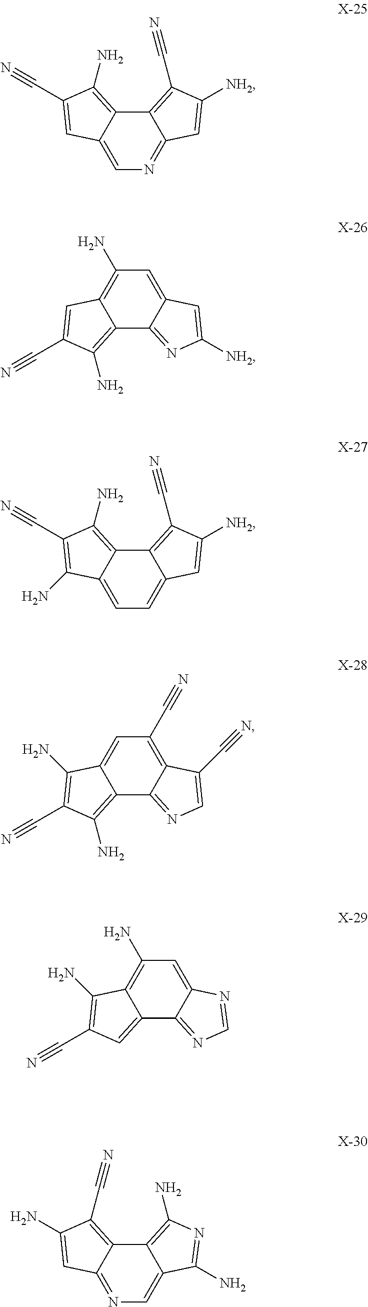 [SMILES: N#CC1=Cc2cnc3c(c2=C1N)=C(C#N)C(N)=C3 ; N#CC1=Cc2c(N)cc3c(c2=C1N)=NC(N)=C3 ; N#CC1=C(N)c2ccc3c(c2=C1N)=C(C#N)C(N)=C3 ; N#CC1=CN=c2c1c(C#N)cc1c2=C(N)C(C#N)=C1N ; N#CC1=C(N)c2c(N)cc3c(c2=C1)=NC=N3 ; N#CC1=c2c(ncc3c2=C(N)N=C3N)C=C1N]

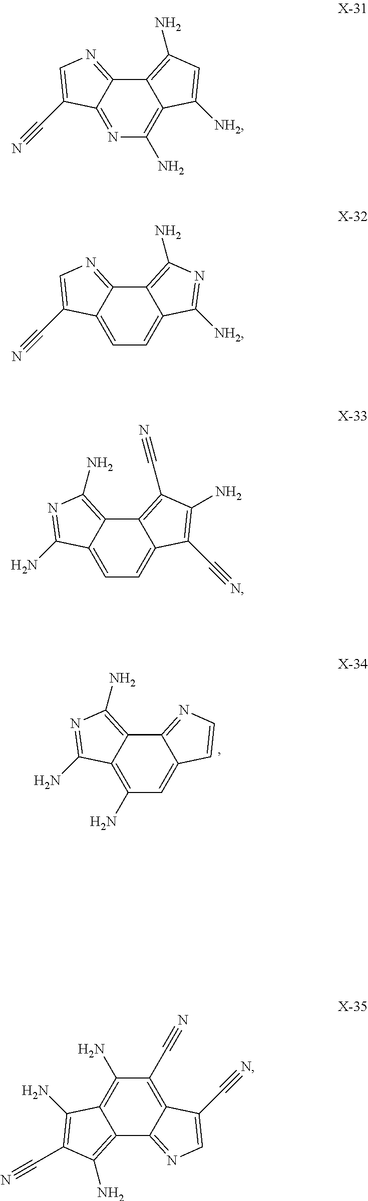 [SMILES: N#CC1=CN=c2c1nc(N)c1c2=C(N)C=C1N ; N#CC1=CN=c2c1ccc1c2=C(N)N=C1N ; N#CC1=C(N)C(C#N)=c2c1ccc1c2=C(N)N=C1N ; NC1=NC(N)=c2c1c(N)cc1c2=NC=C1 ; N#CC1=CN=c2c1c(C#N)c(N)c1c2=C(N)C(C#N)=C1N]

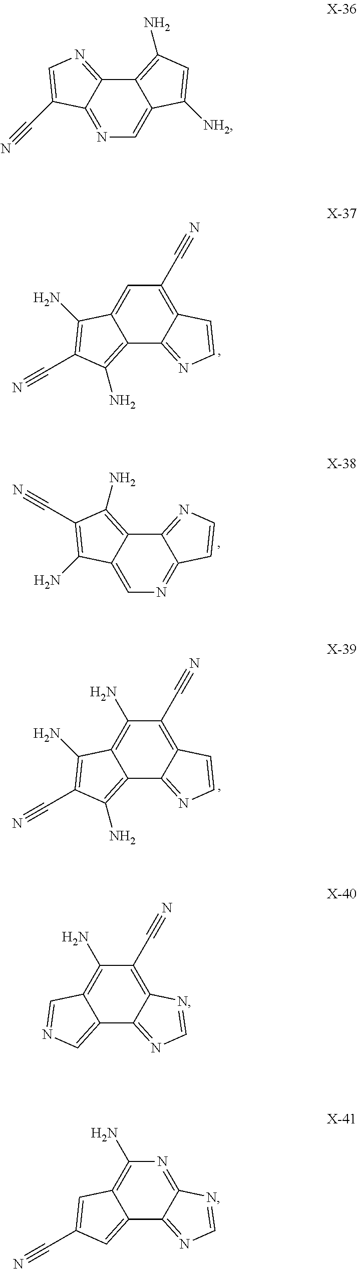 [SMILES: N#CC1=CN=c2c1ncc1c2=C(N)C=C1N ; N#CC1=C(N)c2cc(C#N)c3c(c2=C1N)=NC=C3 ; N#CC1=C(N)c2cnc3c(c2=C1N)=NC=C3 ; N#CC1=C(N)c2c(N)c(C#N)c3c(c2=C1N)=NC=C3 ; N#Cc1c(N)c2c(c3c1N=CN=3)=CN=C2 ; N#CC1=Cc2c(N)nc3c(c2=C1)=NC=N3]

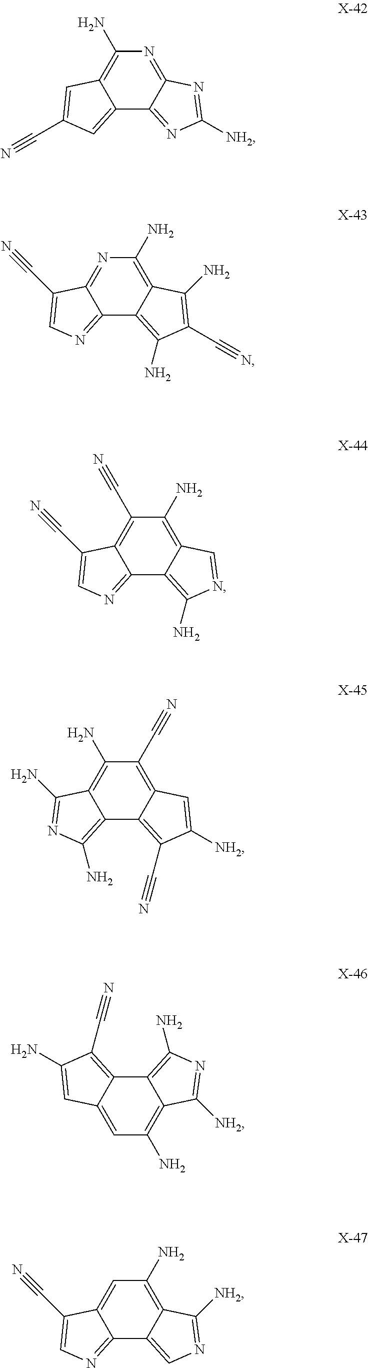 [SMILES: N#CC1=Cc2c(N)nc3c(c2=C1)=NC(N)=N3 ; N#CC1=CN=c2c1nc(N)c1c2=C(N)C(C#N)=C1N ; N#CC1=CN=c2c1c(C#N)c(N)c1c2=C(N)N=C1 ; N#CC1=c2c(c(C#N)c(N)c3c2=C(N)N=C3N)C=C1N ; N#CC1=c2c(cc(N)c3c2=C(N)N=C3N)C=C1N ; N#CC1=CN=c2c1cc(N)c1c2=CN=C1N]

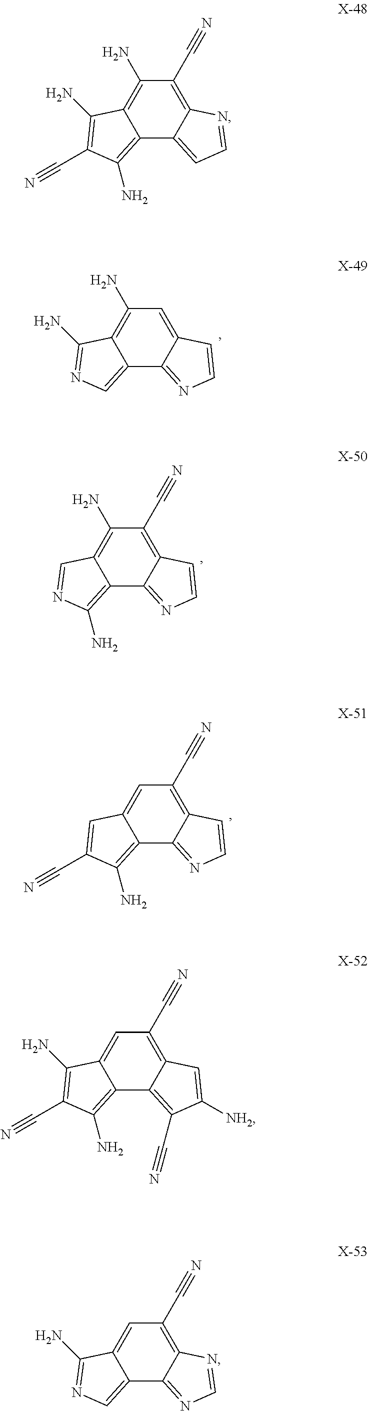 [SMILES: N#CC1=C(N)c2c(N)c(C#N)c3c(c2=C1N)=CC=N3 ; NC1=NC=c2c1c(N)cc1c2=NC=C1 ; N#Cc1c(N)c2c(c3c1C=CN=3)=C(N)N=C2 ; N#CC1=Cc2cc(C#N)c3c(c2=C1N)=NC=C3 ; N#CC1=C(N)c2cc(C#N)c3c(c2=C1N)=C(C#N)C(N)=C3 ; N#Cc1cc2c(c3c1N=CN=3)=CN=C2N]

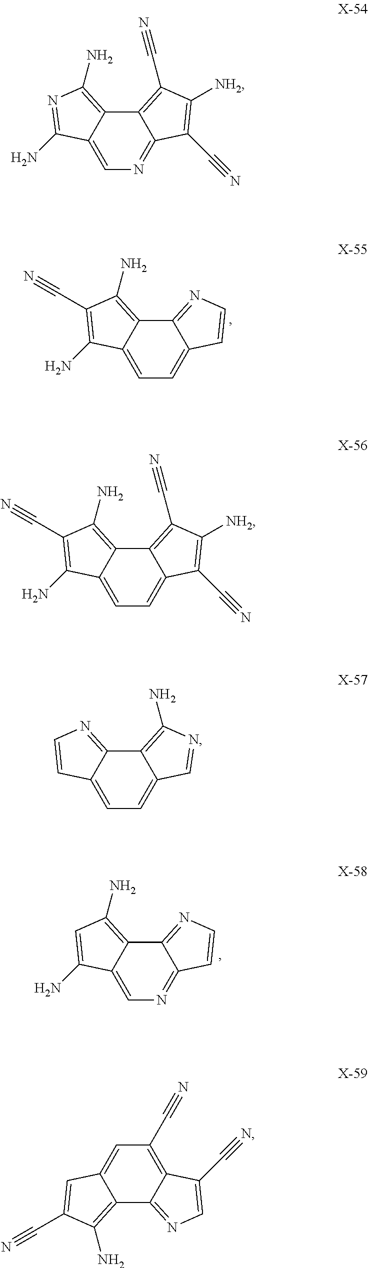 [SMILES: N#CC1=C(N)C(C#N)=c2c1ncc1c2=C(N)N=C1N ; N#CC1=C(N)c2ccc3c(c2=C1N)=NC=C3 ; N#CC1=C(N)c2ccc3c(c2=C1N)=C(C#N)C(N)=C3C#N ; NC1=c2c(ccc3c2=NC=C3)C=N1 ; NC1=CC(N)=c2c1cnc1c2=NC=C1 ; N#CC1=Cc2cc(C#N)c3c(c2=C1N)=NC=C3C#N]

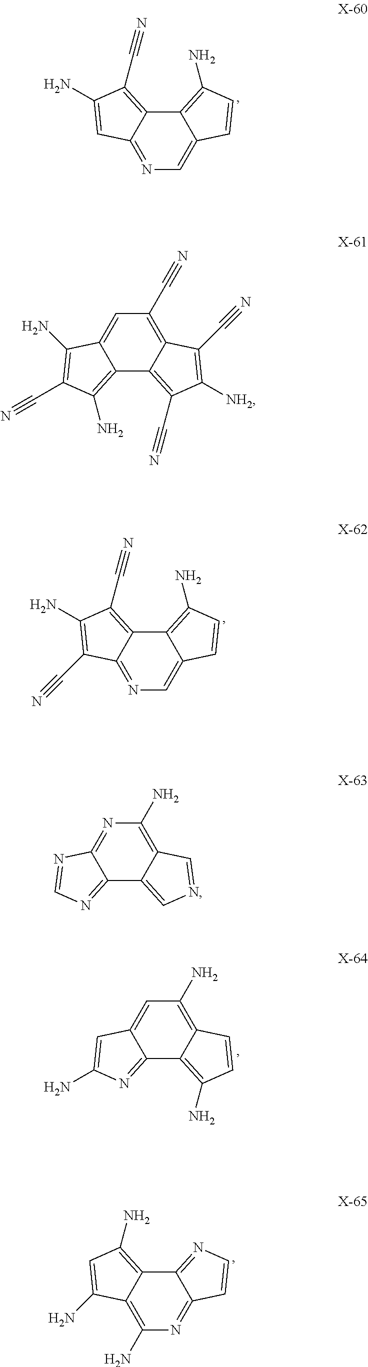 [SMILES: N#CC1=c2c(ncc3c2=C(N)C=C3)C=C1N ; N#CC1=C(N)c2cc(C#N)c3c(c2=C1N)=C(C#N)C(N)=C3C#N ; N#CC1=C(N)C(C#N)=c2c1ncc1c2=C(N)C=C1 ; Nc1nc2c(c3c1C=NC=3)=NC=N2 ; NC1=Cc2cc(N)c3c(c2=N1)=C(N)C=C3 ; NC1=CC(N)=c2c1c(N)nc1c2=NC=C1]

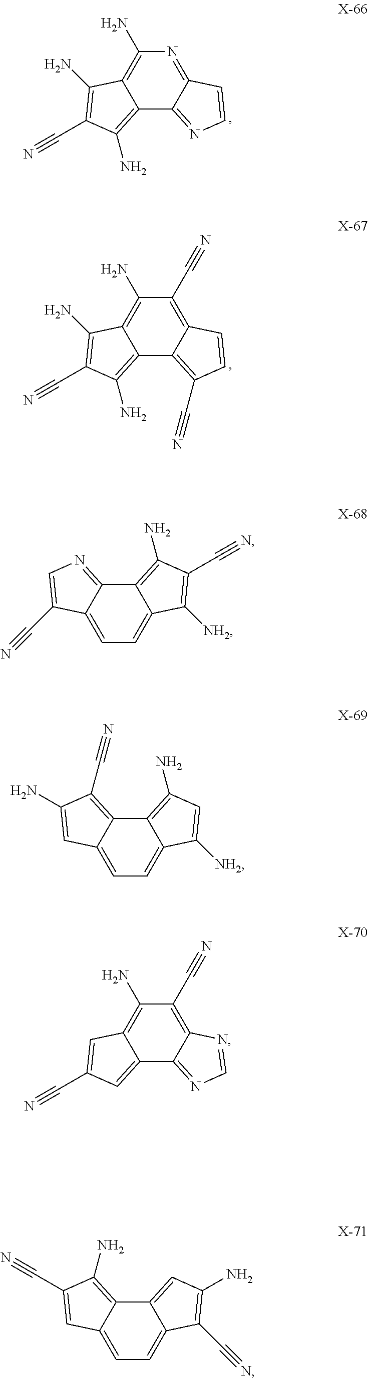 [SMILES: N#CC1=C(N)c2c(N)nc3c(c2=C1N)=NC=C3 ; N#CC1=C(N)c2c(N)c(C#N)c3c(c2=C1N)=C(C#N)C=C3 ; N#CC1=CN=c2c1ccc1c2=C(N)C(C#N)=C1N ; N#CC1=c2c(ccc3c2=C(N)C=C3N)C=C1N ; N#CC1=Cc2c(N)c(C#N)c3c(c2=C1)=NC=N3 ; N#CC1=Cc2ccc3c(c2=C1N)=CC(N)=C3C#N]

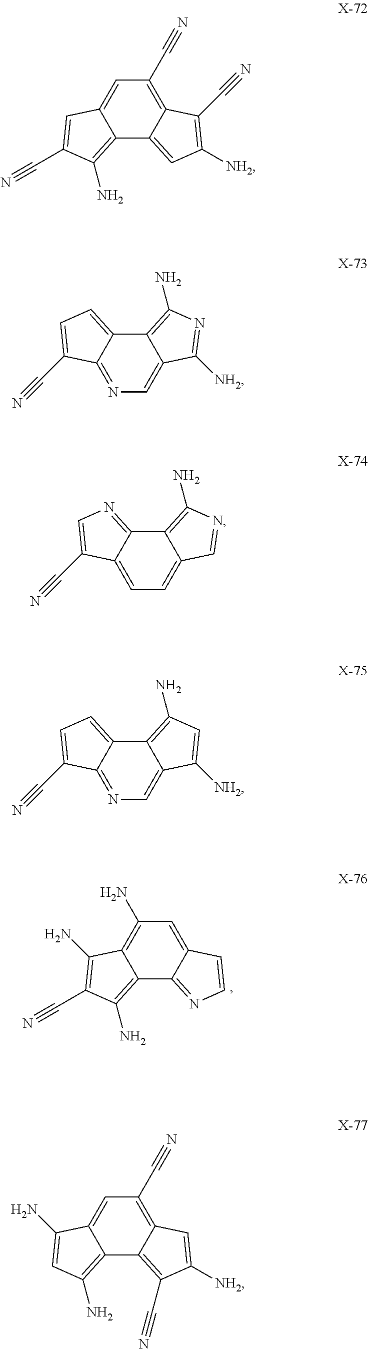 [SMILES: N#CC1=Cc2cc(C#N)c3c(c2=C1N)=CC(N)=C3C#N ; N#CC1=CC=c2c1ncc1c2=C(N)N=C1N ; N#CC1=CN=c2c1ccc1c2=C(N)N=C1 ; N#CC1=CC=c2c1ncc1c2=C(N)C=C1N ; N#CC1=C(N)c2c(N)cc3c(c2=C1N)=NC=C3 ; N#CC1=c2c(c(C#N)cc3c2=C(N)C=C3N)C=C1N]

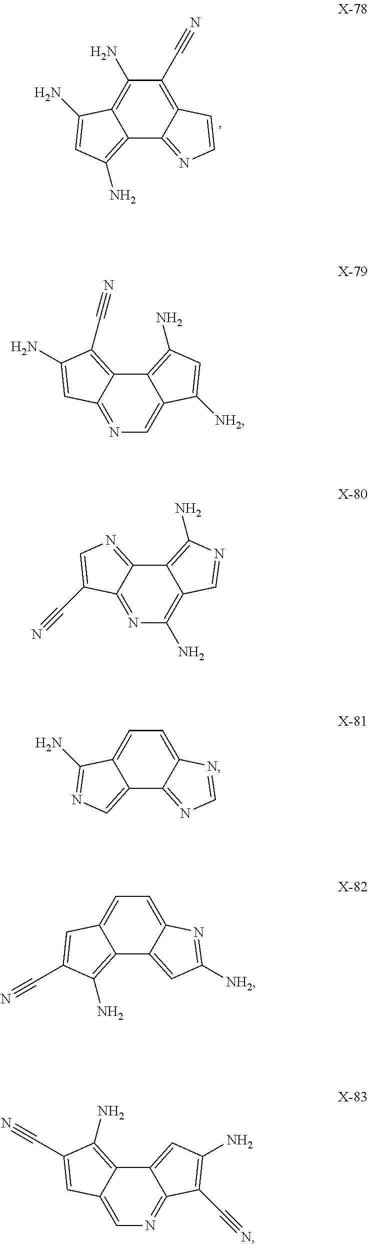 [SMILES: N#Cc1c(N)c2c(c3c1C=CN=3)=C(N)C=C2N ; N#CC1=c2c(ncc3c2=C(N)C=C3N)C=C1N ; N#CC1=CN=c2c1nc(N)c1c2=C(N)N=C1 ; NC1=NC=c2c1ccc1c2=NC=N1 ; N#CC1=Cc2ccc3c(c2=C1N)=CC(N)=N3 ; N#CC1=Cc2cnc3c(c2=C1N)=CC(N)=C3C#N]

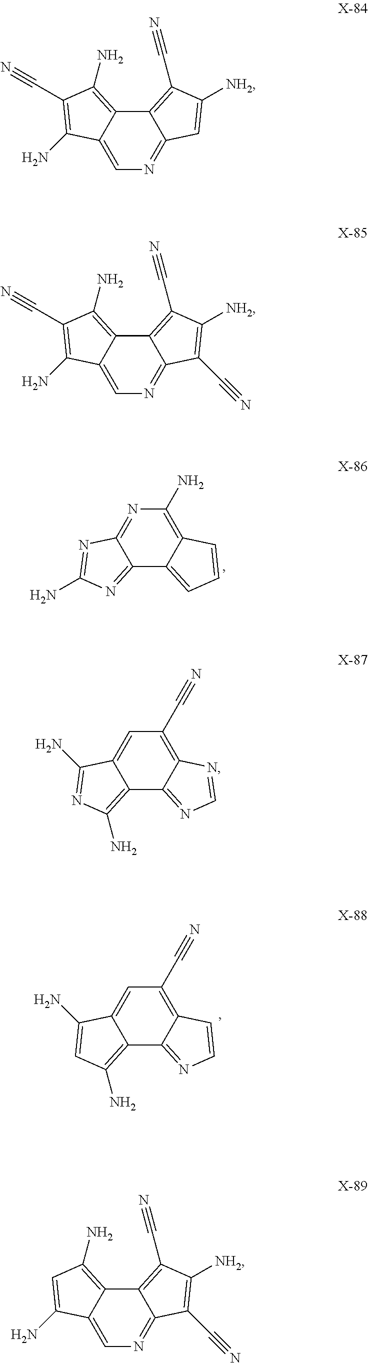 [SMILES: N#CC1=C(N)c2cnc3c(c2=C1N)=C(C#N)C(N)=C3 ; N#CC1=C(N)c2cnc3c(c2=C1N)=C(C#N)C(N)=C3C#N ; NC1=Nc2nc(N)c3c(c2=N1)=CC=C3 ; N#Cc1cc2c(c3c1N=CN=3)=C(N)N=C2N ; N#Cc1cc2c(c3c1C=CN=3)=C(N)C=C2N ; N#CC1=C(N)C(C#N)=c2c1ncc1c2=C(N)C=C1N]

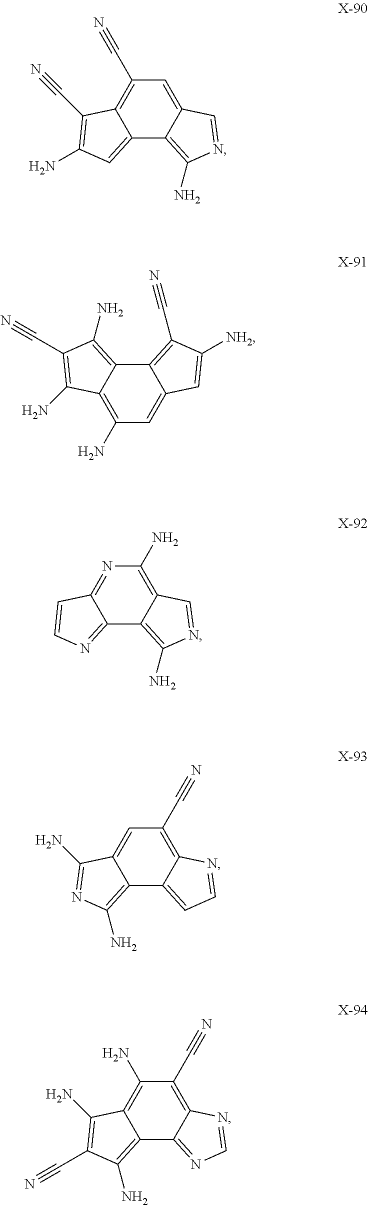 [SMILES: N#CC1=C(N)C=c2c1c(C#N)cc1c2=C(N)N=C1 ; N#CC1=C(N)c2c(N)cc3c(c2=C1N)=C(C#N)C(N)=C3 ; NC1=c2c(c(N)nc3c2=NC=C3)C=N1 ; N#Cc1cc2c(c3c1N=CC=3)=C(N)N=C2N ; N#CC1=C(N)c2c(N)c(C#N)c3c(c2=C1N)=NC=N3]

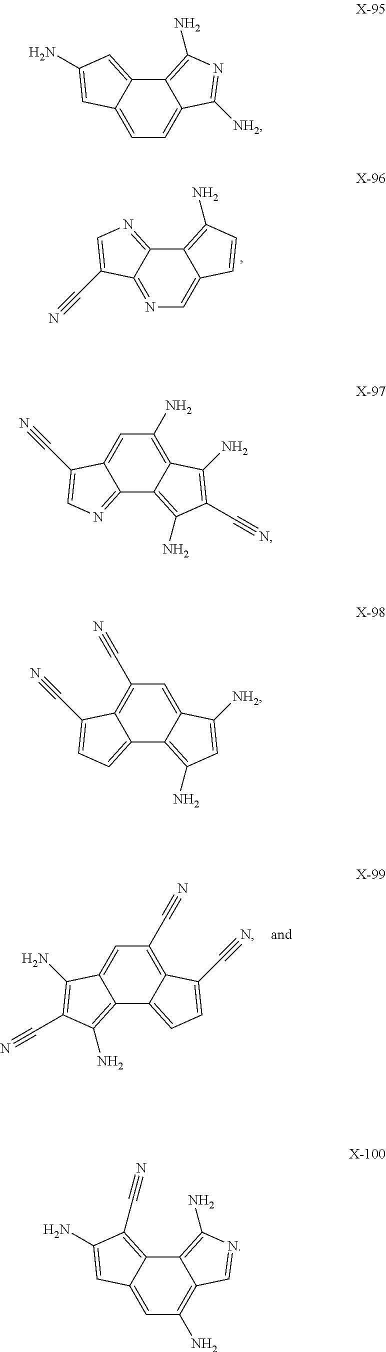 [SMILES: NC1=Cc2ccc3c(c2=C1)=C(N)N=C3N ; N#CC1=CN=c2c1ncc1c2=C(N)C=C1 ; N#CC1=CN=c2c1cc(N)c1c2=C(N)C(C#N)=C1N ; N#CC1=CC=c2c1c(C#N)cc1c2=C(N)C=C1N ; N#CC1=CC=c2c1c(C#N)cc1c2=C(N)C(C#N)=C1N ; N#CC1=c2c(cc(N)c3c2=C(N)N=C3)C=C1N]

Some exemplary compounds of Family XI are shown below:

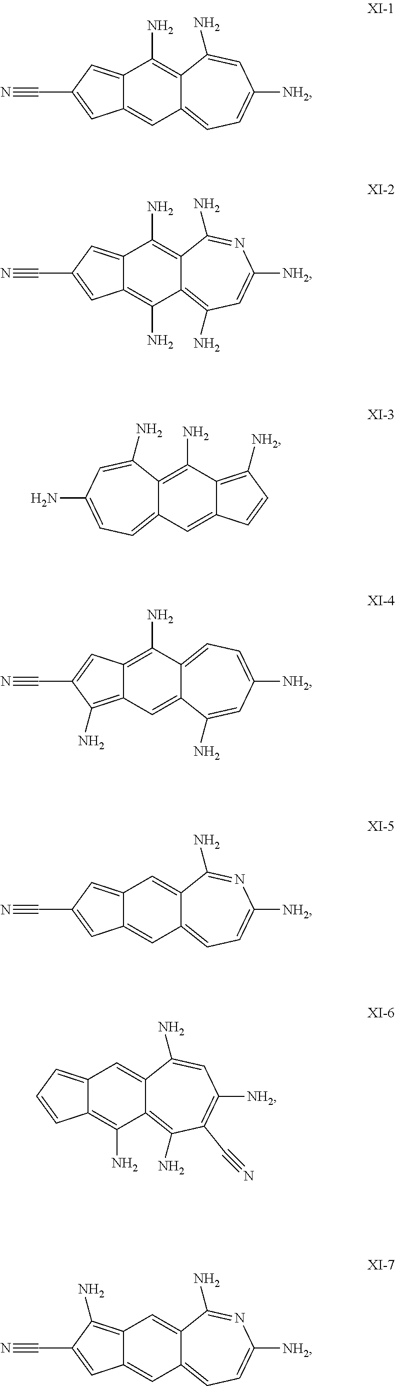

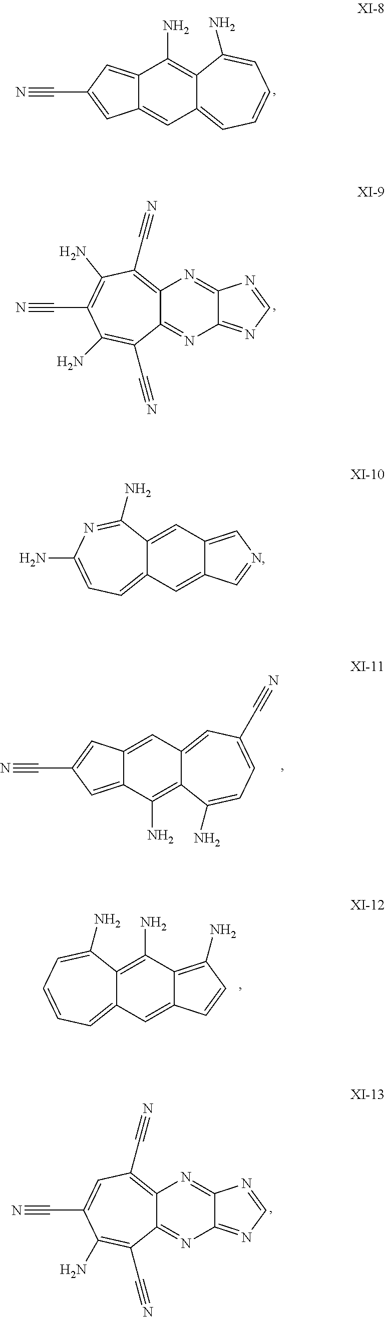 [SMILES: N#Cc1cc2cc3ccccc(N)c3c(N)c2c1 ; N#Cc1c(N)c(C#N)c2nc3ncnc3nc2c(C#N)c1N ; Nc1ccc2cc3cncc3cc2c(N)n1 ; N#Cc1ccc(N)c2c(N)c3cc(C#N)cc3cc2c1 ; Nc1ccccc2cc3ccc(N)c3c(N)c12 ; N#Cc1cc(C#N)c2nc3ncnc3nc2c(C#N)c1N]

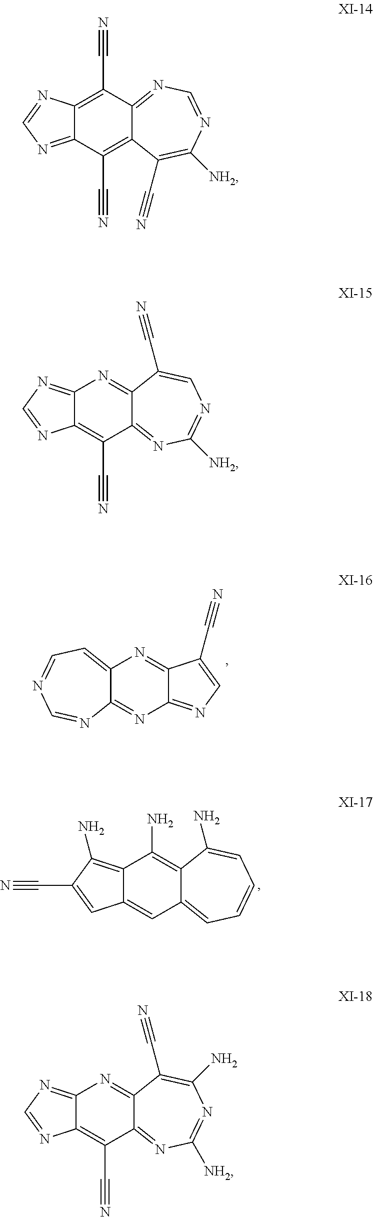 [SMILES: N#Cc1c2ncnc2c(C#N)c2c(C#N)c(N)ncnc12 ; N#Cc1cnc(N)nc2c(C#N)c3ncnc3nc12 ; N#Cc1cnc2nc3ncnccc3nc12 ; N#Cc1cc2cc3ccccc(N)c3c(N)c2c1N ; N#Cc1c(N)nc(N)nc2c(C#N)c3ncnc3nc12]

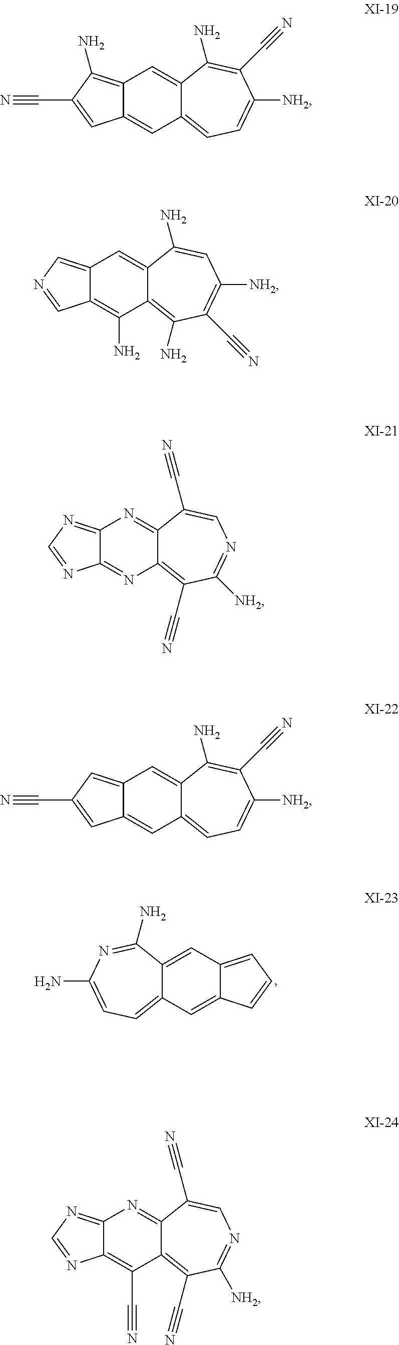 [SMILES: N#Cc1cc2cc3ccc(N)c(C#N)c(N)c3cc2c1N ; N#Cc1c(N)cc(N)c2cc3cncc3c(N)c2c1N ; N#Cc1cnc(N)c(C#N)c2nc3ncnc3nc12 ; N#Cc1cc2cc3ccc(N)c(C#N)c(N)c3cc2c1 ; Nc1ccc2cc3cccc3cc2c(N)n1 ; N#Cc1cnc(N)c(C#N)c2c(C#N)c3ncnc3nc12]

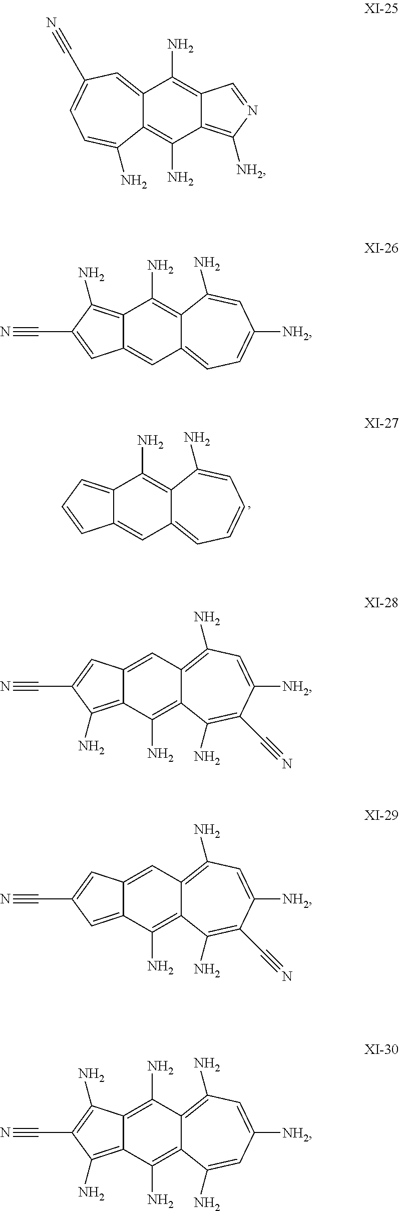 [SMILES: N#Cc1ccc(N)c2c(N)c3c(N)ncc3c(N)c2c1 ; N#Cc1cc2cc3ccc(N)cc(N)c3c(N)c2c1N ; Nc1ccccc2cc3cccc3c(N)c12 ; N#Cc1cc2cc3c(N)cc(N)c(C#N)c(N)c3c(N)c2c1N ; N#Cc1cc2cc3c(N)cc(N)c(C#N)c(N)c3c(N)c2c1 ; N#Cc1c(N)c2c(N)c3c(N)cc(N)cc(N)c3c(N)c2c1N]

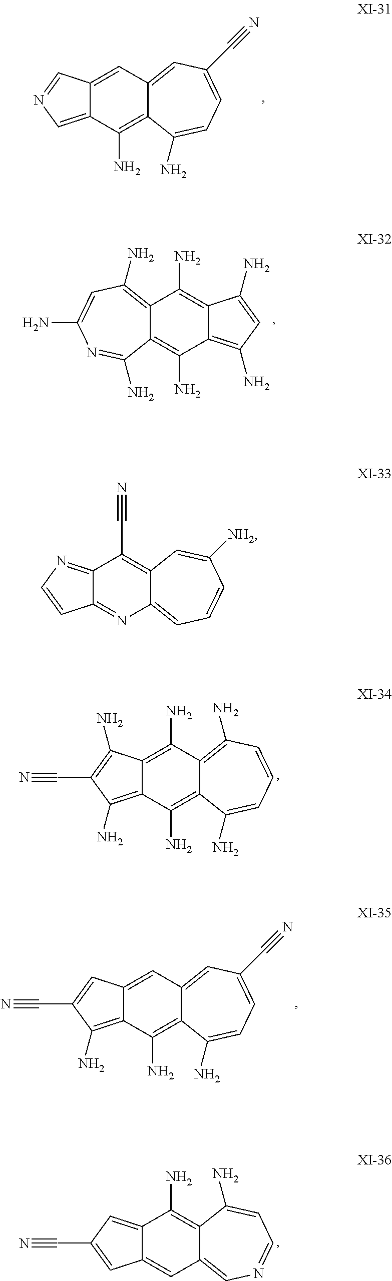 [SMILES: N#Cc1ccc(N)c2c(N)c3cncc3cc2c1 ; Nc1cc(N)c2c(N)c3c(N)cc(N)c3c(N)c2c(N)n1 ; N#Cc1c2cc(N)cccc2nc2ccnc12 ; N#Cc1c(N)c2c(N)c3c(N)cccc(N)c3c(N)c2c1N ; N#Cc1ccc(N)c2c(N)c3c(N)c(C#N)cc3cc2c1 ; N#Cc1cc2cc3cnccc(N)c3c(N)c2c1]

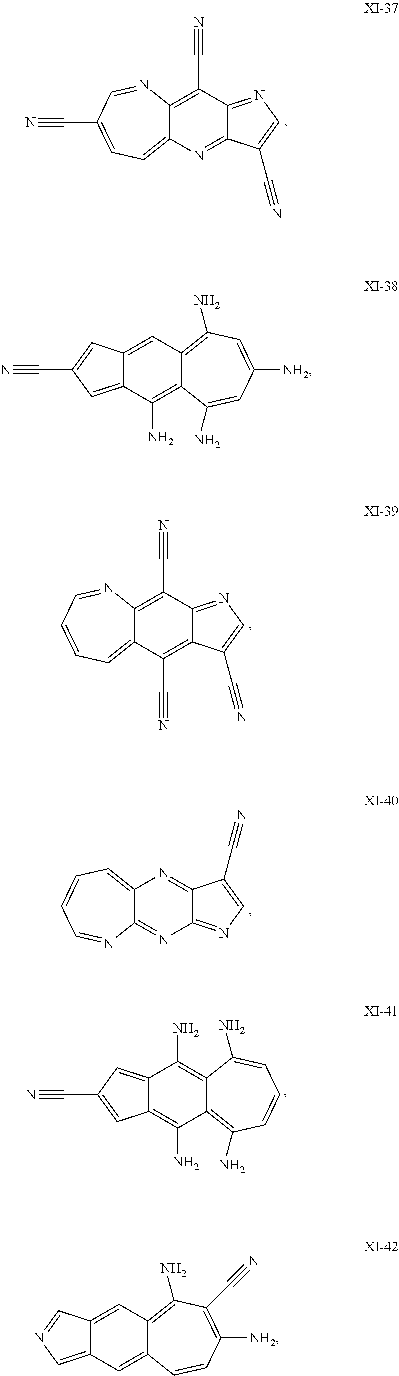 [SMILES: N#Cc1ccc2nc3c(C#N)cnc3c(C#N)c2nc1 ; N#Cc1cc2cc3c(N)cc(N)cc(N)c3c(N)c2c1 ; N#Cc1c2nccccc2c(C#N)c2c(C#N)cnc12 ; N#Cc1cnc2nc3nccccc3nc12 ; N#Cc1cc2c(N)c3c(N)cccc(N)c3c(N)c2c1 ; N#Cc1c(N)ccc2cc3cncc3cc2c1N]

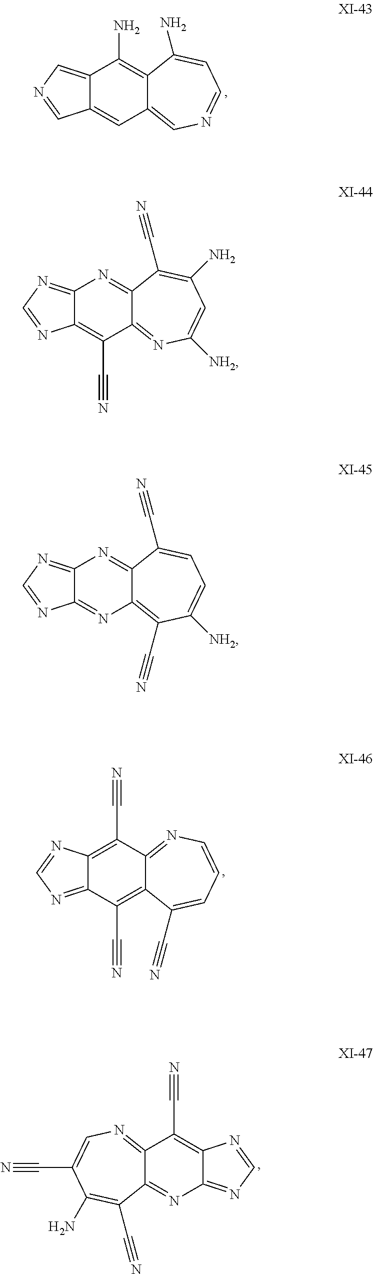 [SMILES: Nc1ccncc2cc3cncc3c(N)c12 ; N#Cc1c(N)cc(N)nc2c(C#N)c3ncnc3nc12 ; N#Cc1ccc(N)c(C#N)c2nc3ncnc3nc12 ; N#Cc1c2ncnc2c(C#N)c2c(C#N)cccnc12 ; N#Cc1cnc2c(C#N)c3ncnc3nc2c(C#N)c1N]

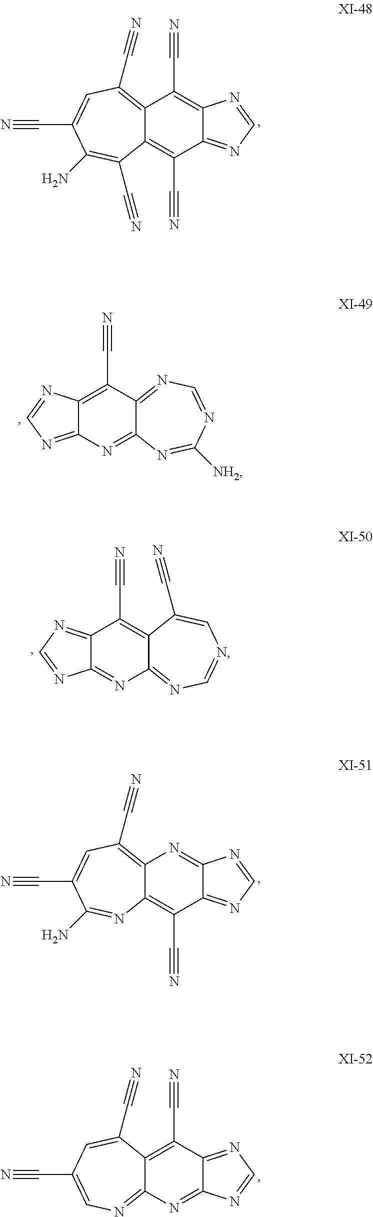 [SMILES: N#Cc1cc(C#N)c2c(C#N)c3ncnc3c(C#N)c2c(C#N)c1N ; N#Cc1c2ncnc2nc2nc(N)ncnc12 ; N#Cc1cncnc2nc3ncnc3c(C#N)c12 ; N#Cc1cc(C#N)c2nc3ncnc3c(C#N)c2nc1N ; N#Cc1cnc2nc3ncnc3c(C#N)c2c(C#N)c1]

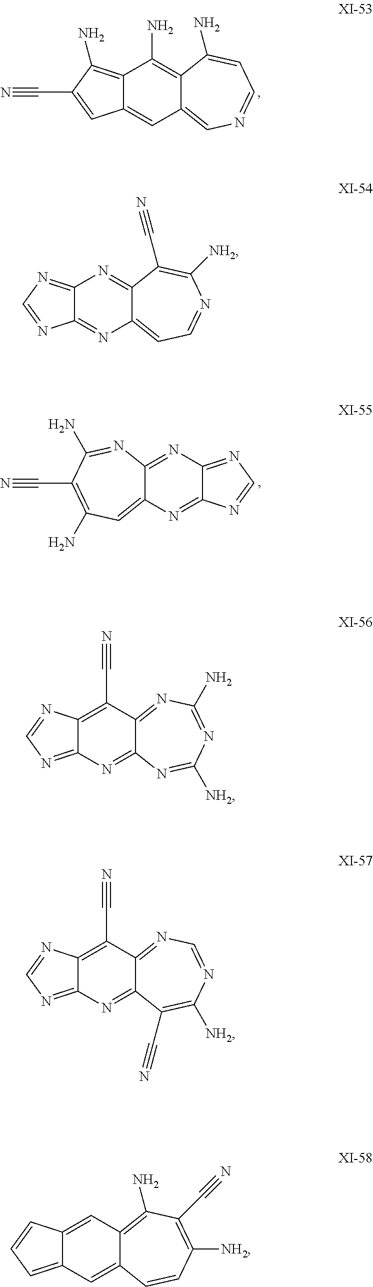 [SMILES: N#Cc1cc2cc3cnccc(N)c3c(N)c2c1N ; N#Cc1c(N)nccc2nc3ncnc3nc12 ; N#Cc1c(N)cc2nc3ncnc3nc2nc1N ; N#Cc1c2ncnc2nc2nc(N)nc(N)nc12 ; N#Cc1c(N)ncnc2c(C#N)c3ncnc3nc12 ; N#Cc1c(N)ccc2cc3cccc3cc2c1N]

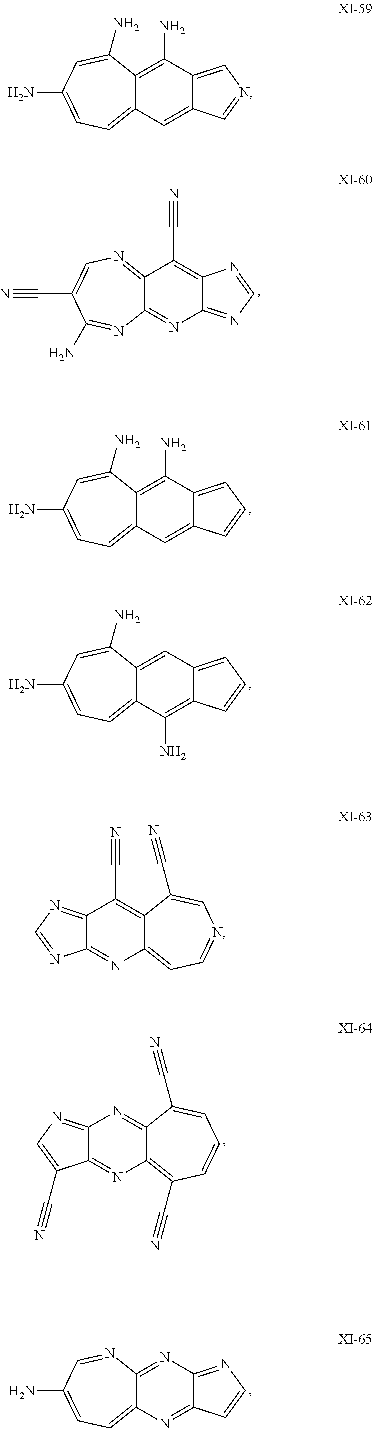

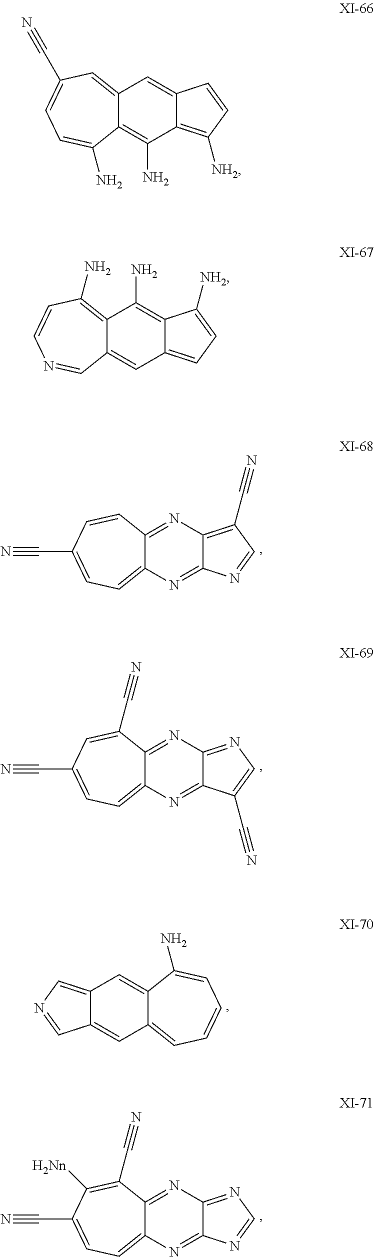

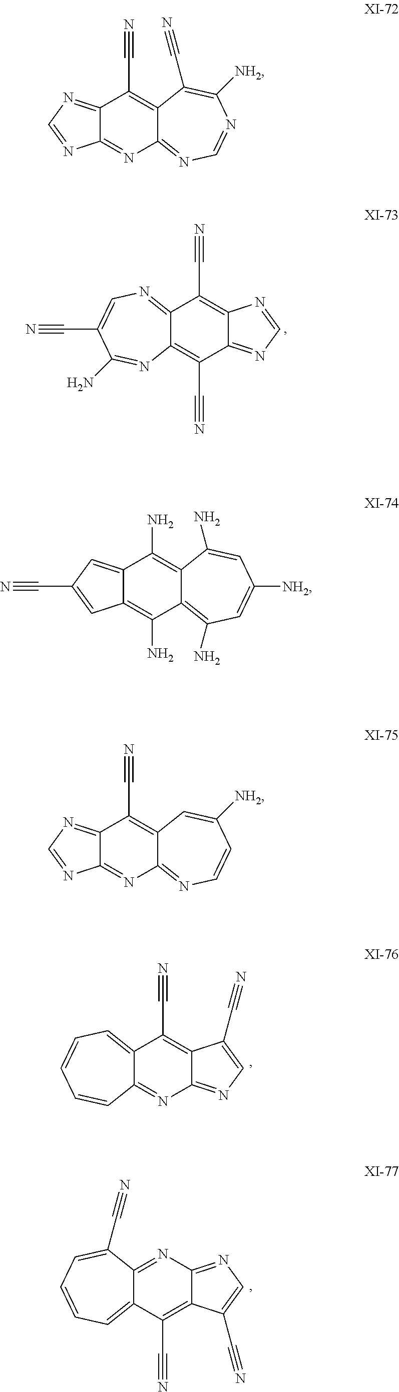 [SMILES: N#Cc1c(N)ncnc2nc3ncnc3c(C#N)c12 ; N#Cc1cnc2c(C#N)c3ncnc3c(C#N)c2nc1N ; N#Cc1cc2c(N)c3c(N)cc(N)cc(N)c3c(N)c2c1 ; N#Cc1c2cc(N)ccnc2nc2ncnc12 ; N#Cc1cnc2nc3cccccc3c(C#N)c12 ; N#Cc1ccccc2c(C#N)c3c(C#N)cnc3nc12]

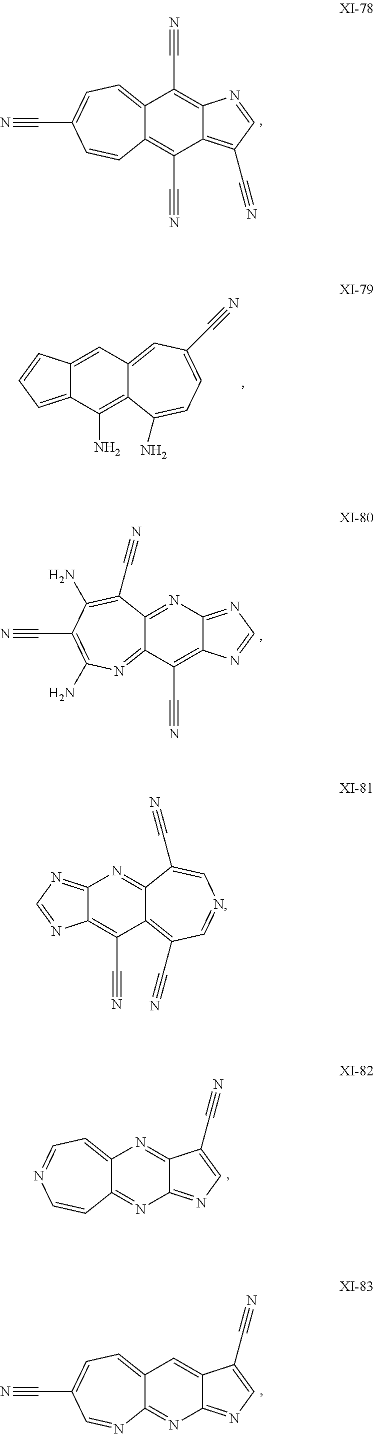 [SMILES: N#Cc1ccc2c(C#N)c3ncc(C#N)c3c(C#N)c2cc1 ; N#Cc1ccc(N)c2c(N)c3cccc3cc2c1 ; N#Cc1c(N)nc2c(C#N)c3ncnc3nc2c(C#N)c1N ; N#Cc1cncc(C#N)c2c(C#N)c3ncnc3nc12 ; N#Cc1cnc2nc3ccnccc3nc12 ; N#Cc1ccc2cc3c(C#N)cnc3nc2nc1]

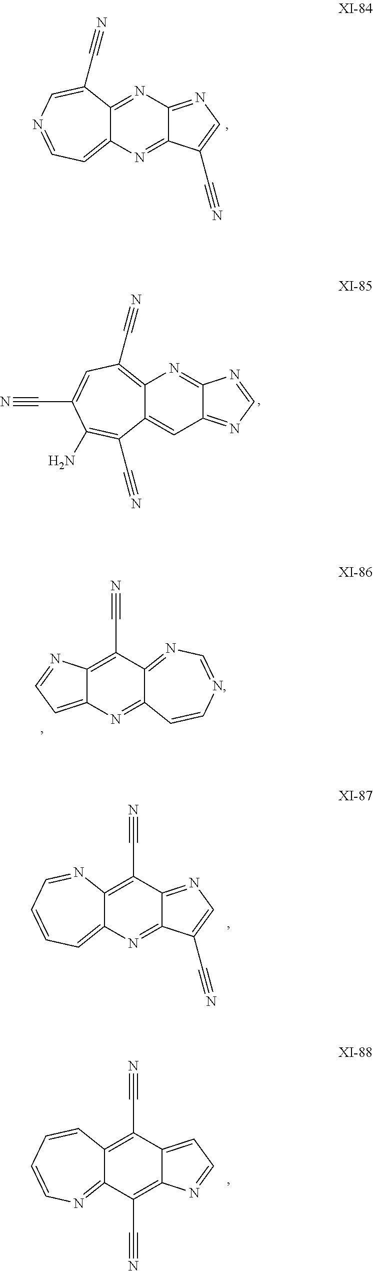 [SMILES: N#Cc1cnccc2nc3c(C#N)cnc3nc12 ; N#Cc1cc(C#N)c2nc3ncnc3cc2c(C#N)c1N ; N#Cc1c2nccc2nc2ccncnc12 ; N#Cc1cnc2c(C#N)c3nccccc3nc12 ; N#Cc1c2ccccnc2c(C#N)c2nccc12]

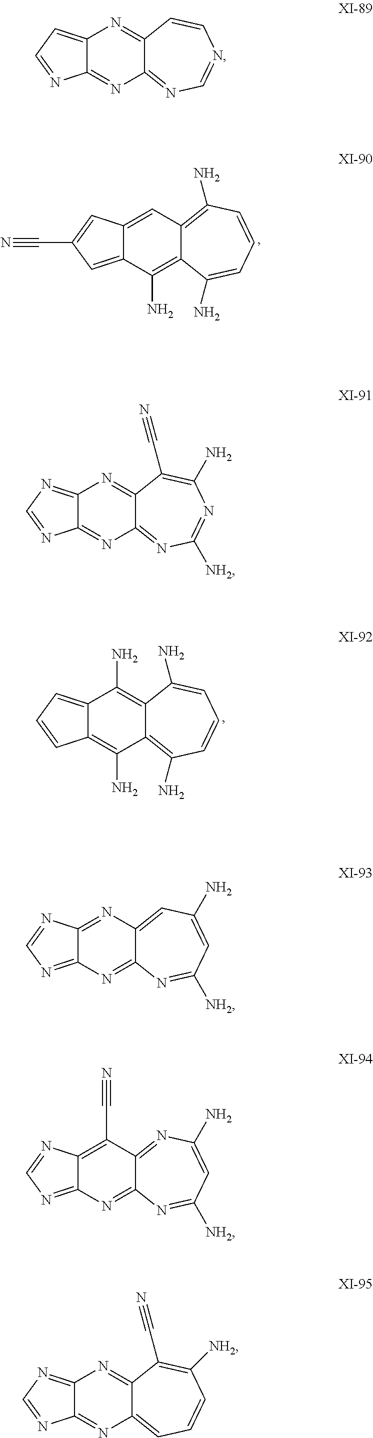

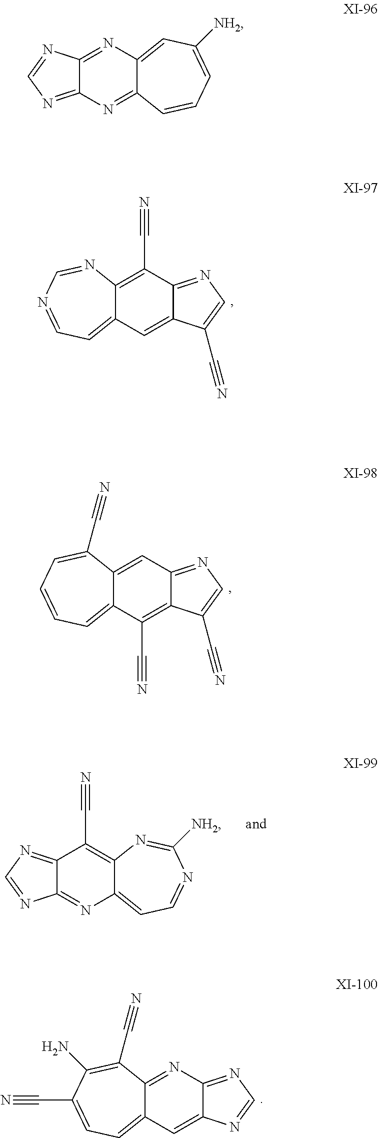 [SMILES: Nc1cccc2nc3ncnc3nc2c1 ; N#Cc1cnc2c(C#N)c3ncnccc3cc12 ; N#Cc1ccccc2c(C#N)c3c(C#N)cnc3cc12 ; N#Cc1c2nc(N)nccc2nc2ncnc12 ; N#Cc1ccc2cc3ncnc3nc2c(C#N)c1N]

Some exemplary compounds of Family XIII are shown below:

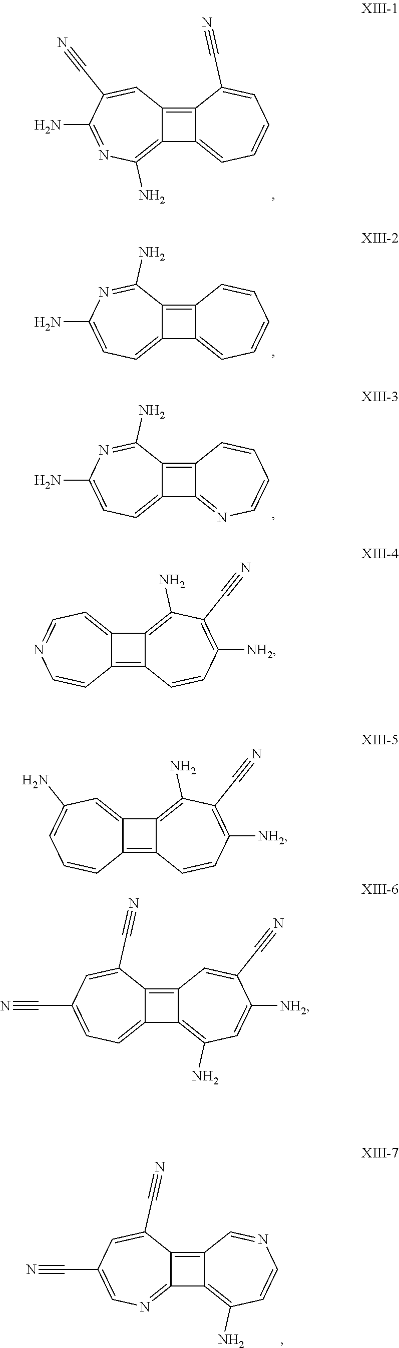

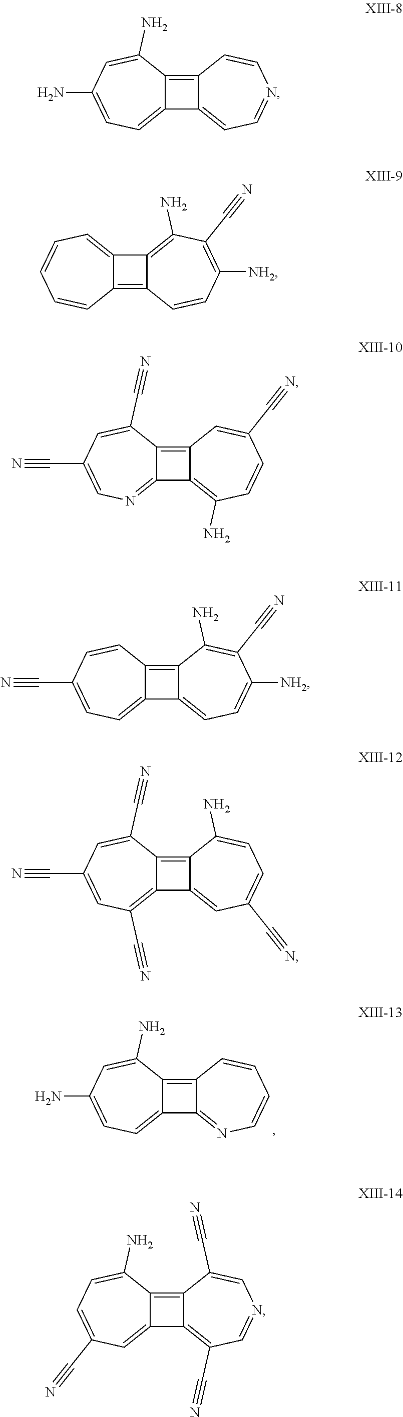

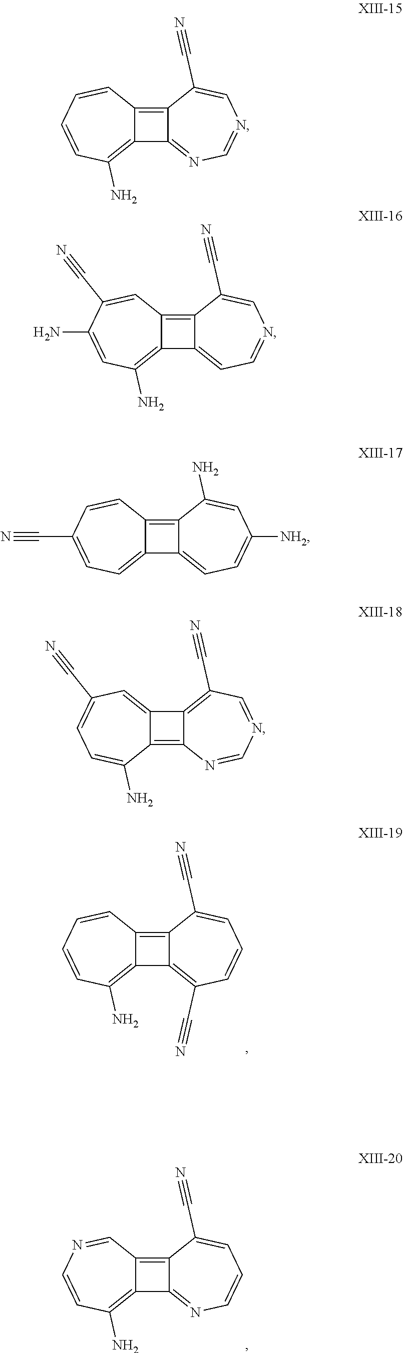 [SMILES: N#Cc1cncnc2c3c(N)ccccc-3c1-2 ; N#Cc1cc2c3c(C#N)cnccc-3c-2c(N)cc1N ; N#Cc1ccc2c3ccc(N)cc(N)c-3c-2cc1 ; N#Cc1ccc(N)c2c3ncncc(C#N)c-3c-2c1 ; N#Cc1cccc(C#N)c2c3c(N)ccccc-3c1-2 ; N#Cc1cccnc2c3c(N)ccncc-3c1-2]

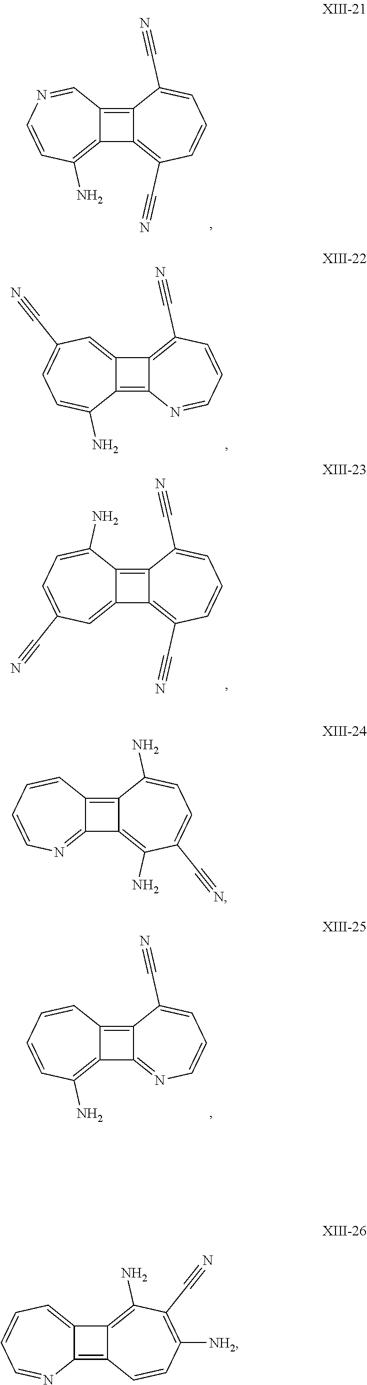 [SMILES: N#Cc1cccc(C#N)c2c3c(N)ccncc-3c1-2 ; N#Cc1ccc(N)c2c3ncccc(C#N)c-3c-2c1 ; N#Cc1ccc(N)c2c3c(C#N)cccc(C#N)c-3c-2c1 ; N#Cc1ccc(N)c2c3ccccnc-3c-2c1N ; N#Cc1cccnc2c3c(N)ccccc-3c1-2 ; N#Cc1c(N)ccc2c3nccccc-3c-2c1N]

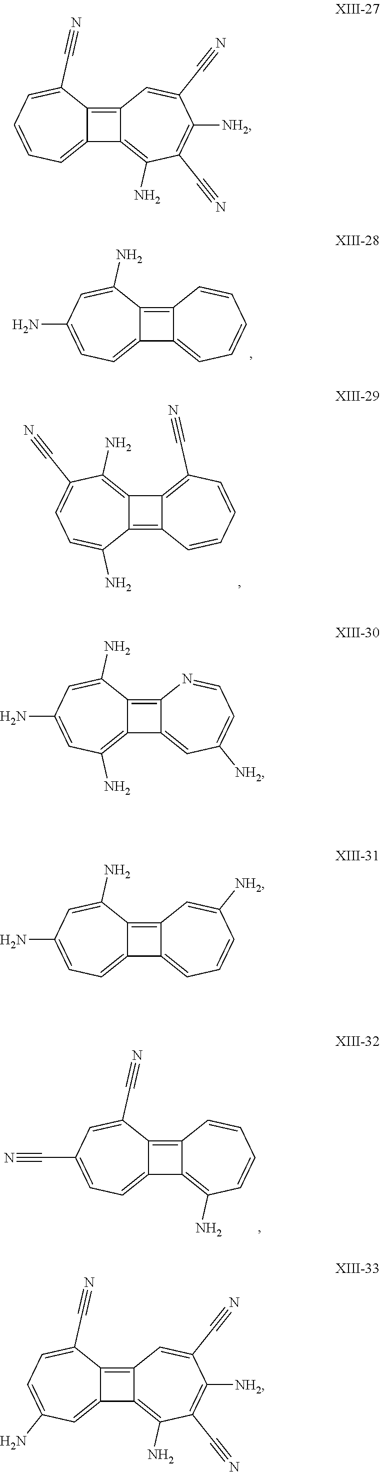

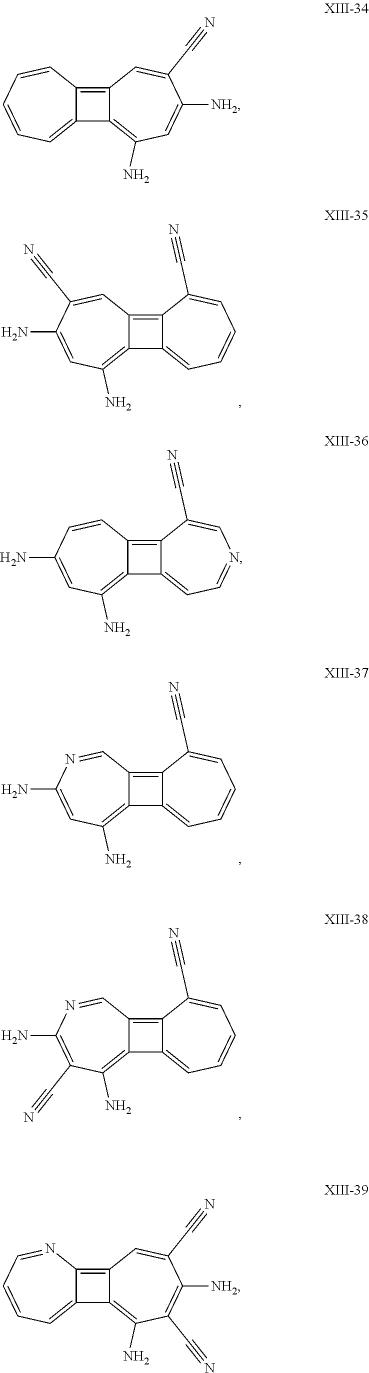 [SMILES: N#Cc1cc2c3cccccc-3c-2c(N)cc1N ; N#Cc1cc2c3c(C#N)ccccc-3c-2c(N)cc1N ; N#Cc1cnccc2c3c(N)cc(N)ccc-3c1-2 ; N#Cc1ccccc2c3c(N)cc(N)ncc-3c1-2 ; N#Cc1ccccc2c3c(N)c(C#N)c(N)ncc-3c1-2 ; N#Cc1cc2c3nccccc-3c-2c(N)c(C#N)c1N]

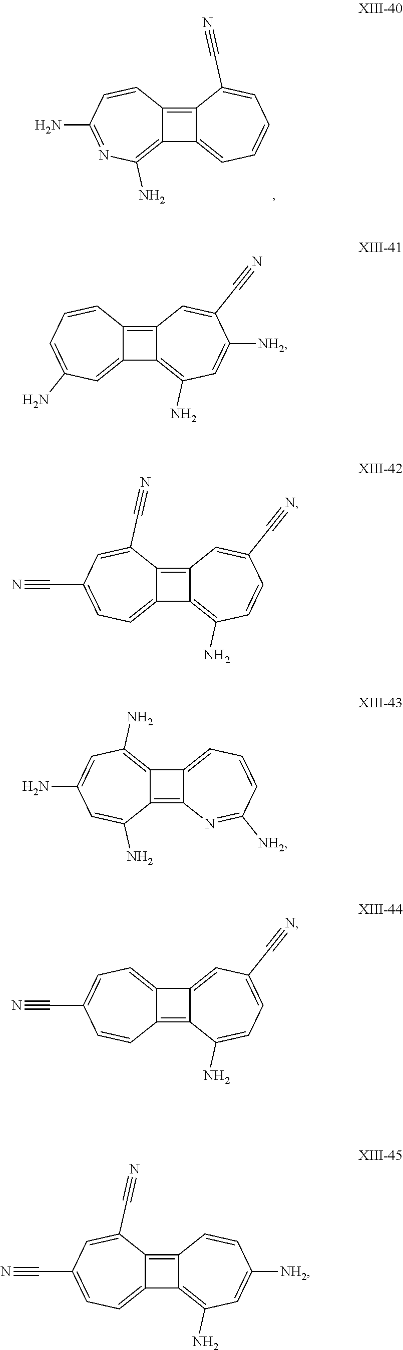 [SMILES: N#Cc1ccccc2c3c(N)nc(N)ccc-3c1-2 ; N#Cc1cc2c3cccc(N)cc-3c-2c(N)cc1N ; N#Cc1ccc2c3c(N)ccc(C#N)cc-3c-2c(C#N)c1 ; Nc1cc(N)c2c3cccc(N)nc-3c-2c(N)c1 ; N#Cc1ccc2c3cc(C#N)ccc(N)c-3c-2cc1 ; N#Cc1ccc2c3c(N)cc(N)ccc-3c-2c(C#N)c1]

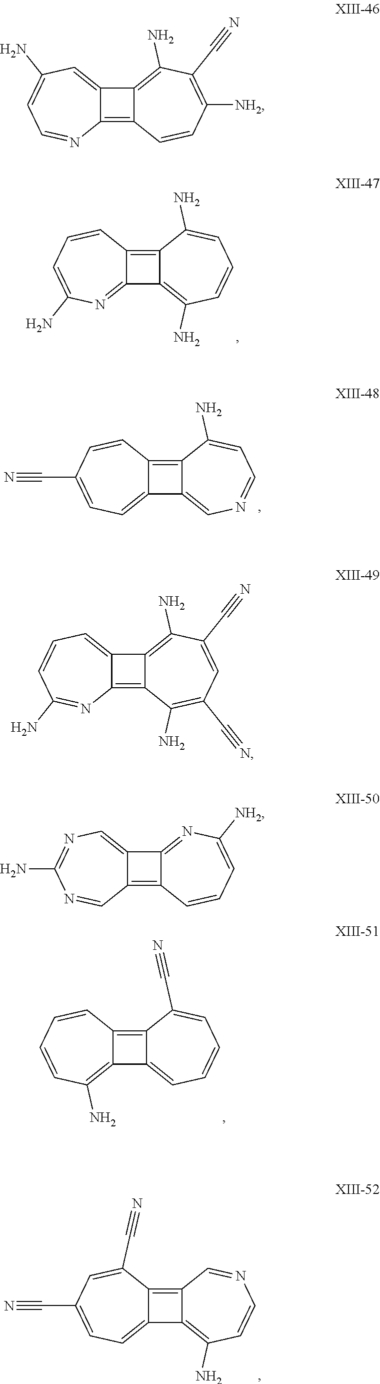

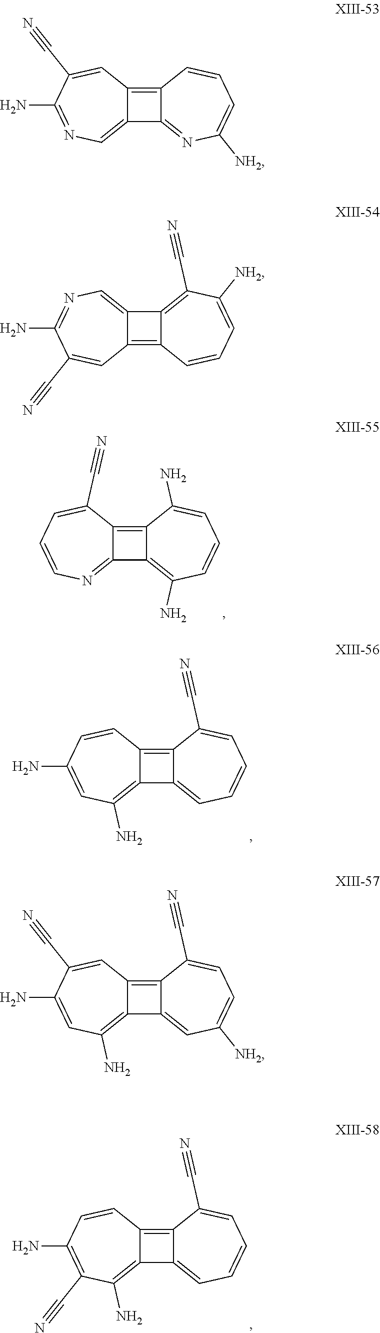 [SMILES: N#Cc1cc2c3cccc(N)nc-3c-2cnc1N ; N#Cc1cc2c3cccc(N)c(C#N)c-3c-2cnc1N ; N#Cc1cccnc2c3c(N)cccc(N)c-3c1-2 ; N#Cc1ccccc2c3c(N)cc(N)ccc-3c1-2 ; N#Cc1cc2c3c(C#N)ccc(N)cc-3c-2c(N)cc1N ; N#Cc1ccccc2c3c(N)c(C#N)c(N)ccc-3c1-2]

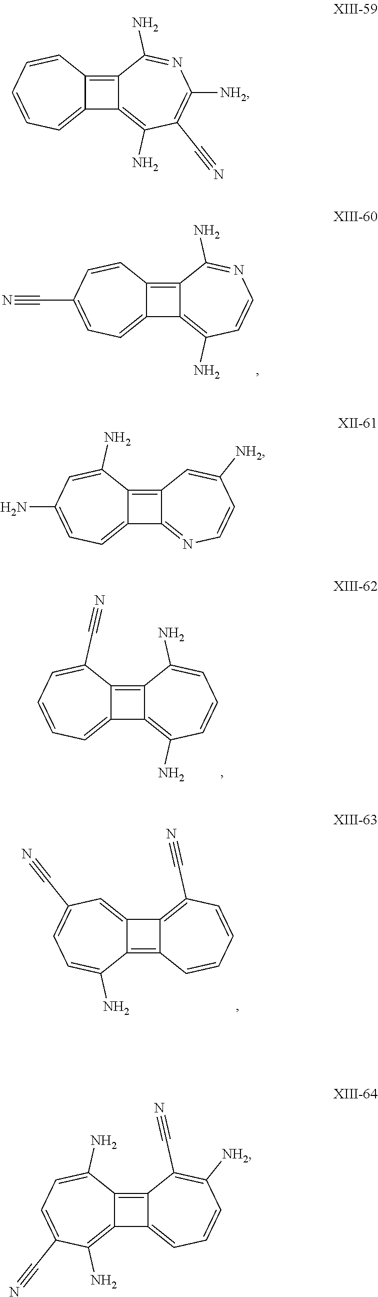 [SMILES: N#Cc1c(N)nc(N)c2c3cccccc-3c-2c1N ; N#Cc1ccc2c3c(N)ccnc(N)c-3c-2cc1 ; Nc1ccc2c3nccc(N)cc-3c-2c(N)c1 ; N#Cc1ccccc2c3c(N)cccc(N)c-3c1-2 ; N#Cc1ccc(N)c2c3ccccc(C#N)c-3c-2c1 ; N#Cc1ccc(N)c2c3c(C#N)c(N)cccc-3c-2c1N]

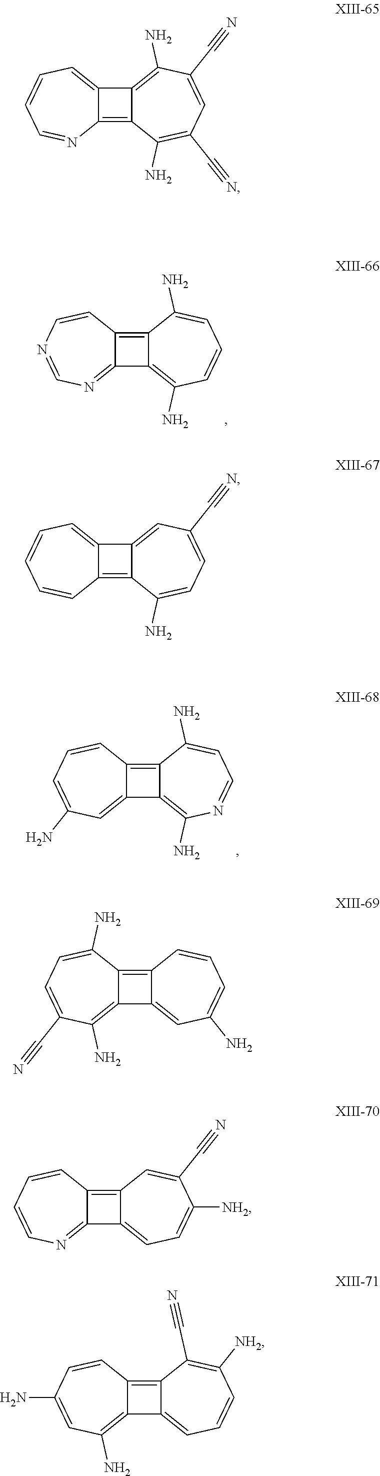

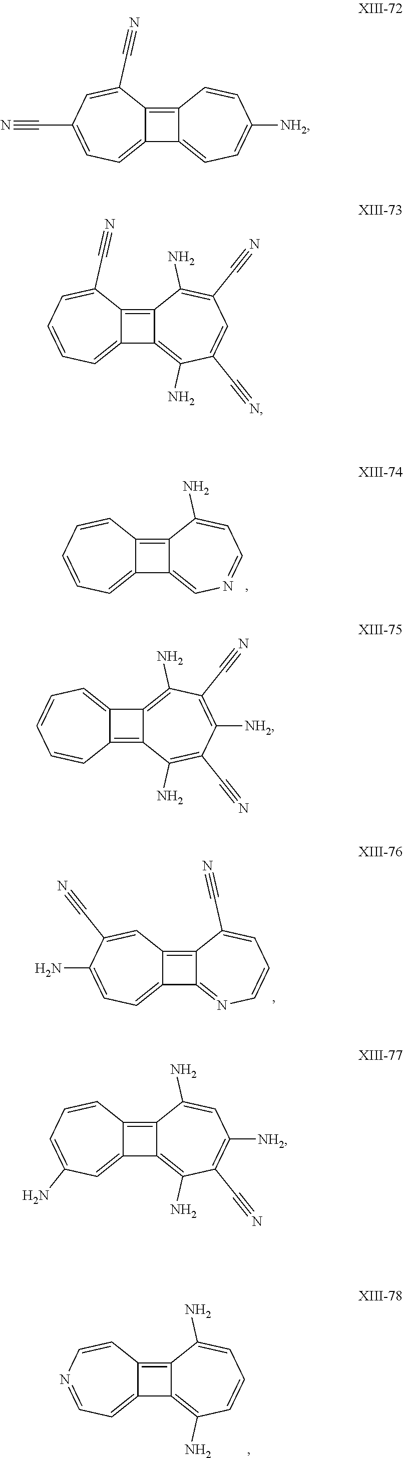

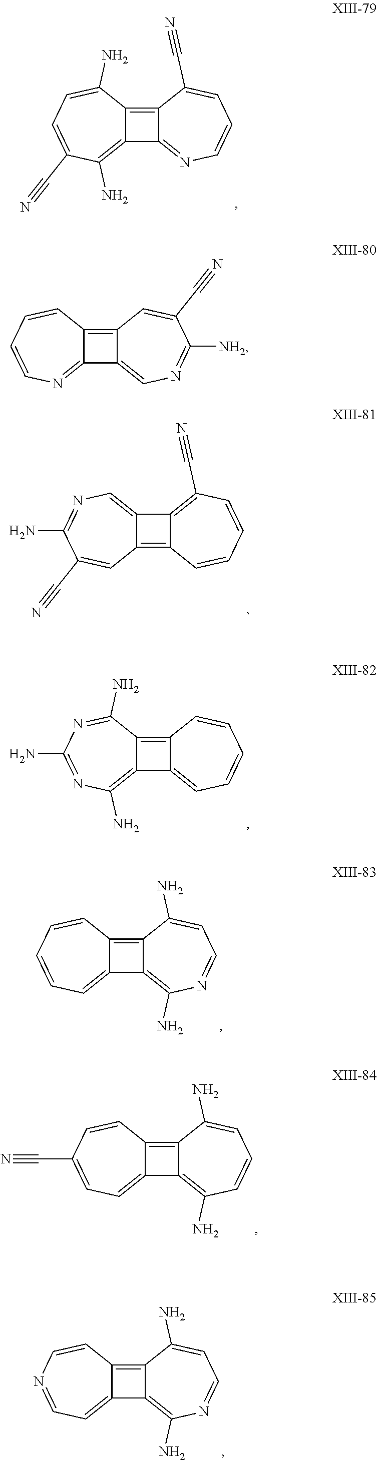

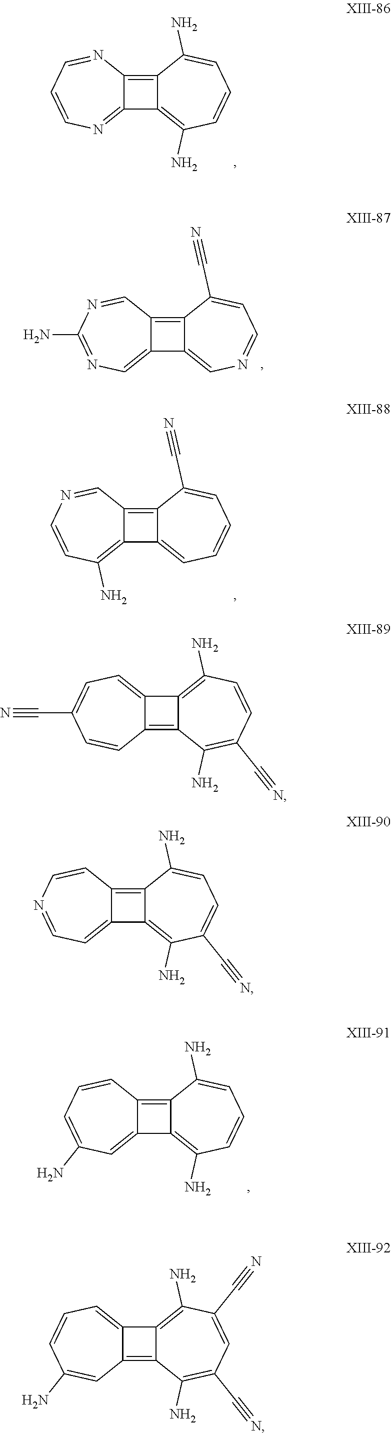

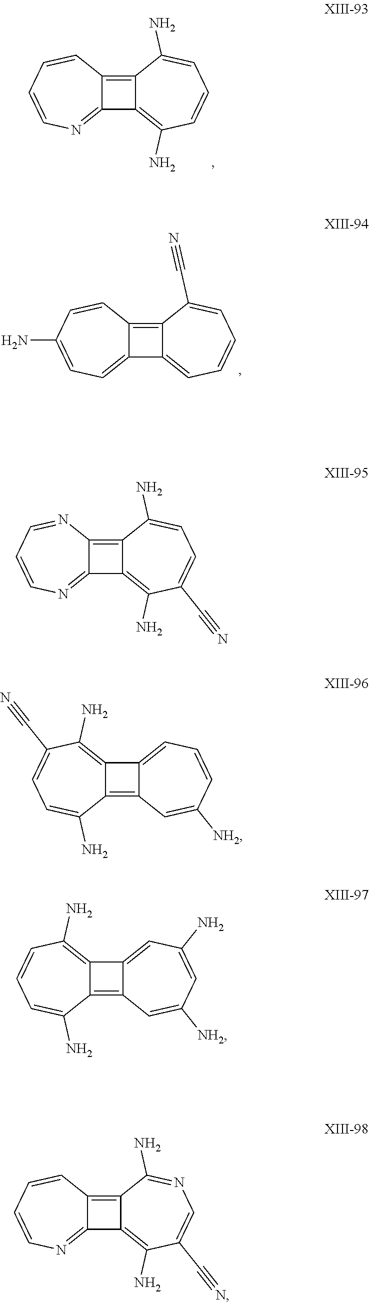 [SMILES: Nc1cccc(N)c2c3nccccc-3c1-2 ; N#Cc1ccccc2c3ccc(N)ccc-3c1-2 ; N#Cc1ccc(N)c2c3ncccnc-3c-2c1N ; N#Cc1ccc(N)c2c3cc(N)cccc-3c-2c1N ; Nc1cc(N)cc2c3c(N)cccc(N)c-3c-2c1 ; N#Cc1cnc(N)c2c3ccccnc-3c-2c1N]

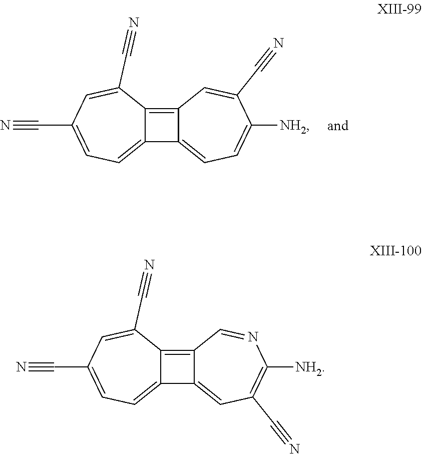 [SMILES: N#Cc1ccc2c3ccc(N)c(C#N)cc-3c-2c(C#N)c1 ; N#Cc1ccc2c3cc(C#N)c(N)ncc-3c-2c(C#N)c1]

Some exemplary compounds of Family XIV are shown below:

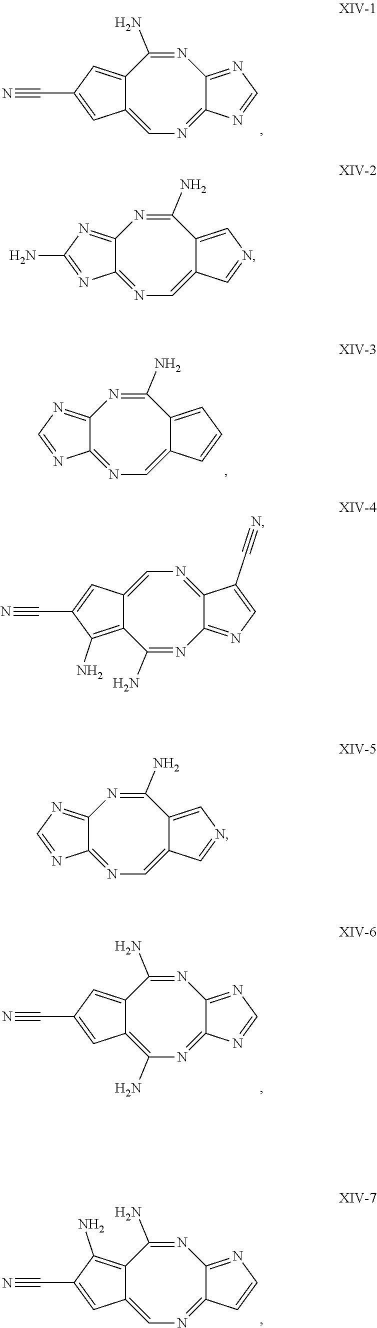

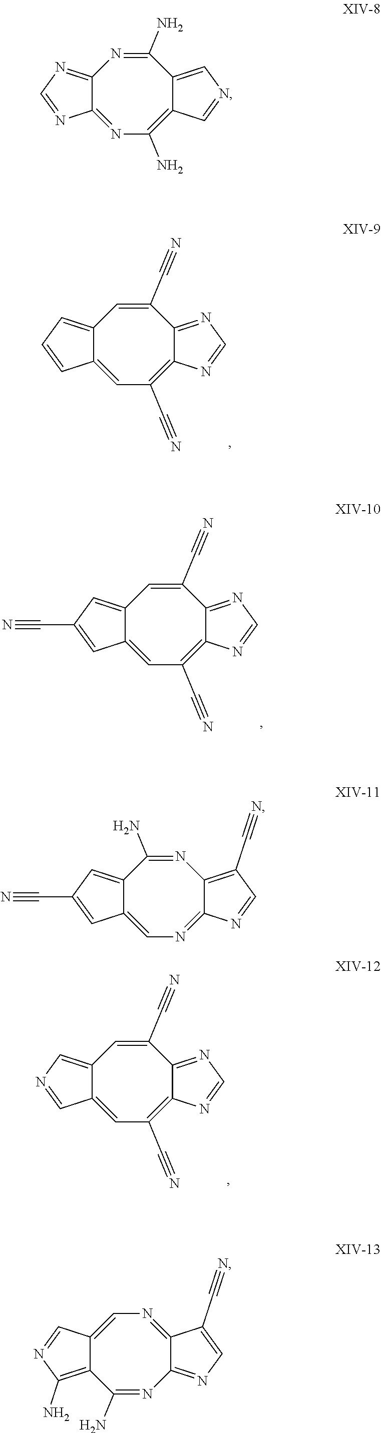

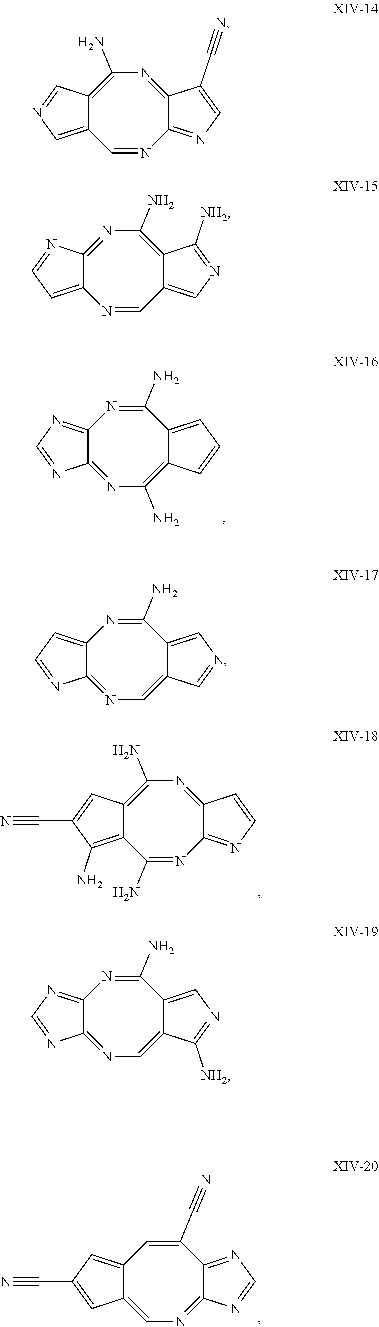

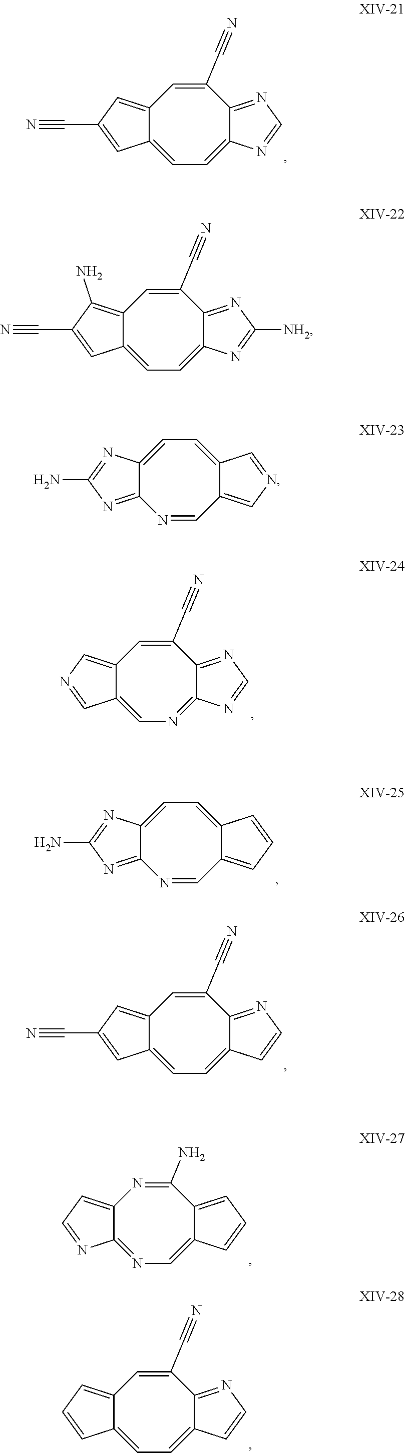

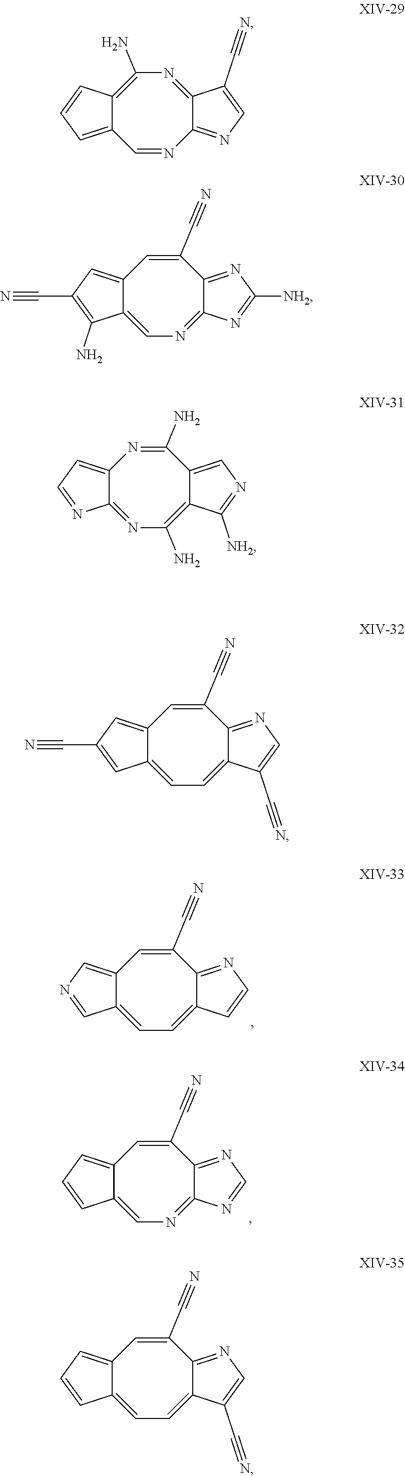

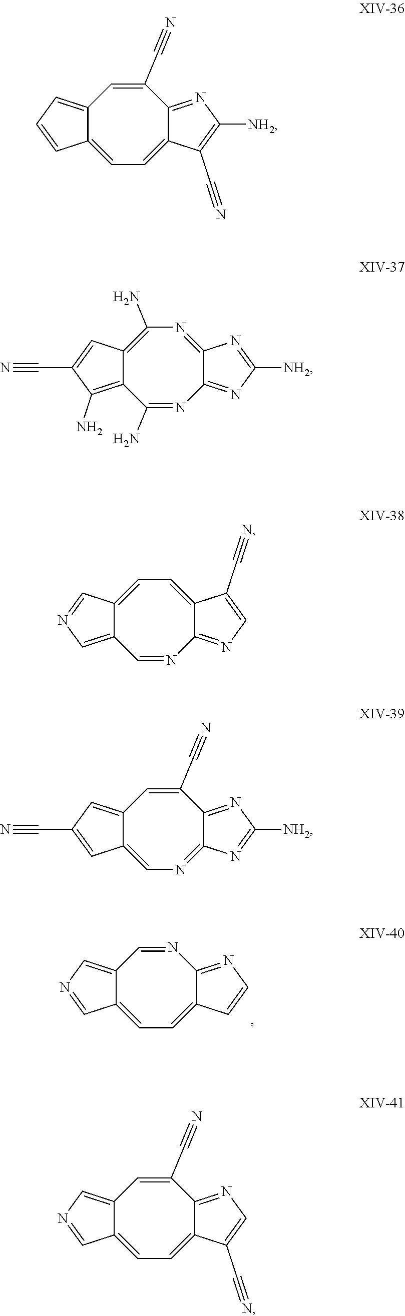 [SMILES: N#Cc1cc2cccc-2ccc2c(C#N)c(N)nc1-2 ; N#Cc1cc2c(N)nc3nc(N)nc-3nc(N)c-2c1N ; N#Cc1cnc2ncc3cncc-3ccc1-2 ; N#Cc1cc2cnc3nc(N)nc-3c(C#N)cc-2c1 ; c1cc2ccc3cncc-3cnc-2n1 ; N#Cc1cnc2c(C#N)cc3cncc-3ccc1-2]

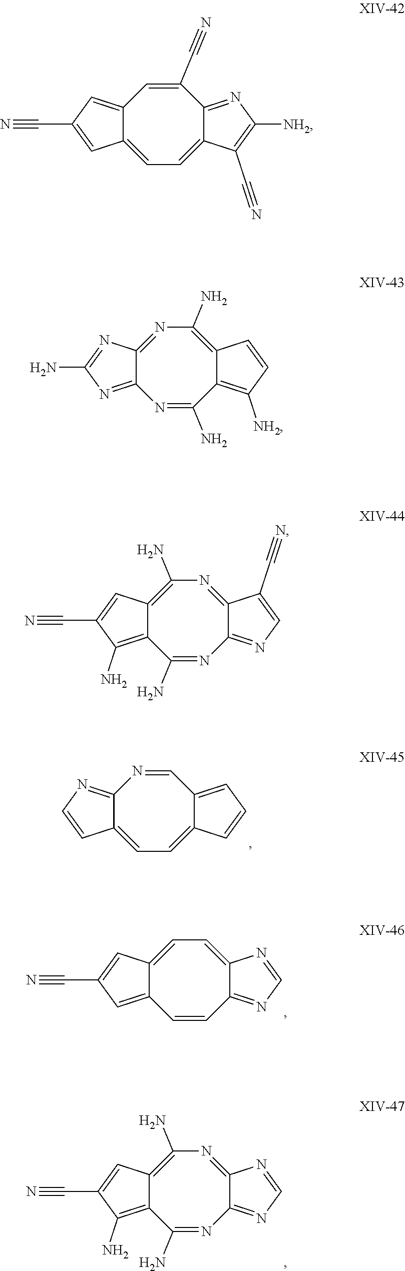 [SMILES: N#Cc1cc2ccc3c(C#N)c(N)nc-3c(C#N)cc-2c1 ; Nc1nc2nc(N)c3ccc(N)c-3c(N)nc-2n1 ; N#Cc1cnc2nc(N)c3c(N)c(C#N)cc-3c(N)nc1-2 ; c1cc2ccc3ccnc-3ncc-2c1 ; N#Cc1cc2ccc3ncnc-3ccc-2c1 ; N#Cc1cc2c(N)nc3ncnc-3nc(N)c-2c1N]

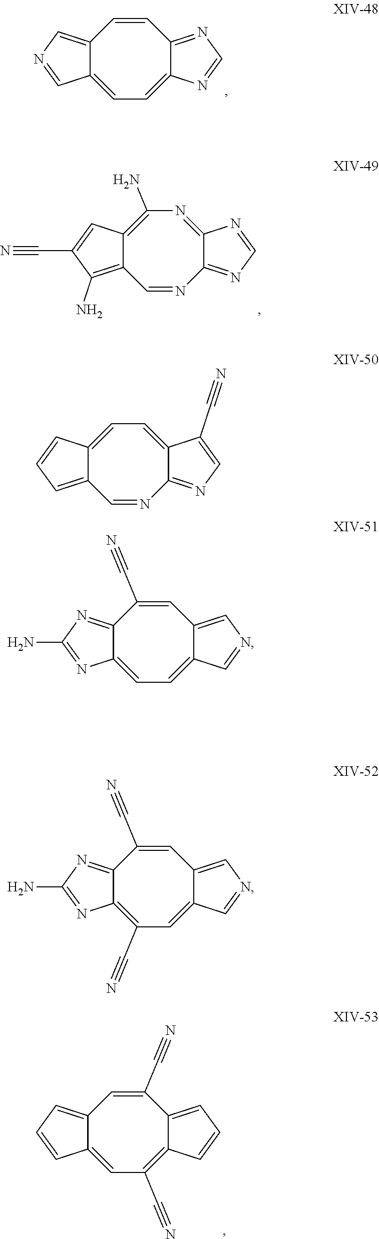

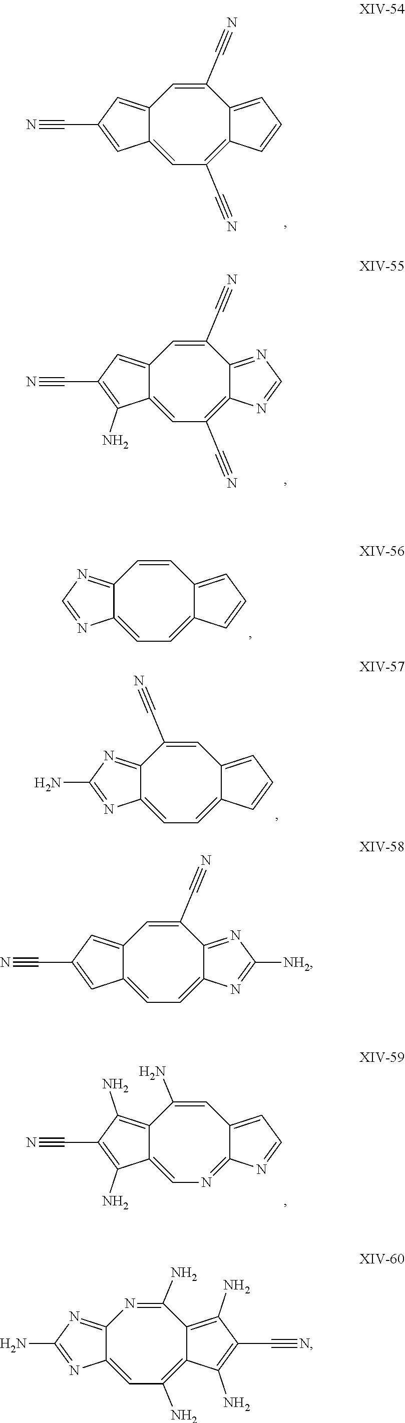

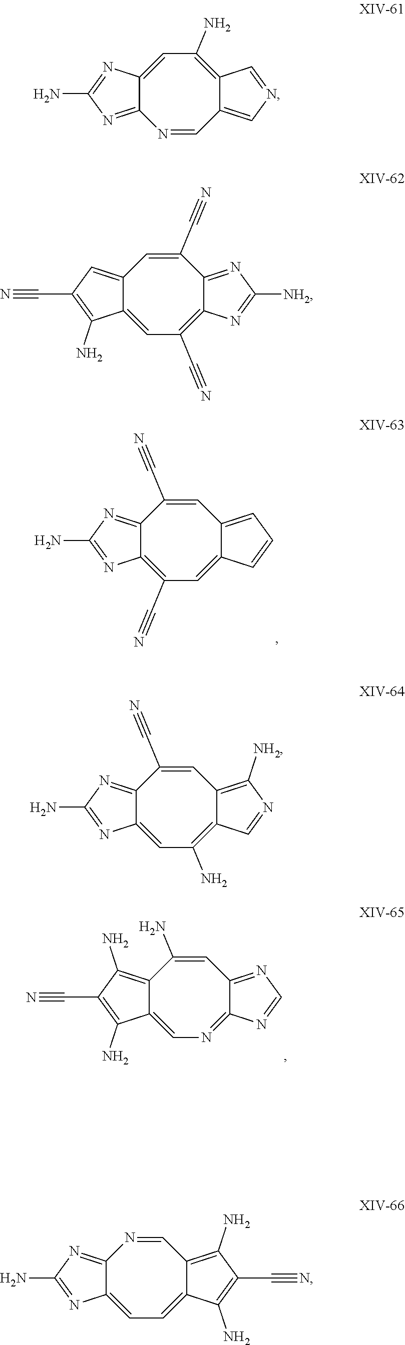 [SMILES: Nc1nc2cc(N)c3cncc-3cnc-2n1 ; N#Cc1cc2cc(C#N)c(N)c-2cc(C#N)c2nc(N)nc1-2 ; N#Cc1cc2cccc-2cc(C#N)c2nc(N)nc1-2 ; N#Cc1cc2c(N)ncc-2c(N)cc2nc(N)nc1-2 ; N#Cc1c(N)c2cnc3ncnc-3cc(N)c-2c1N ; N#Cc1c(N)c2ccc3nc(N)nc-3ncc-2c1N]

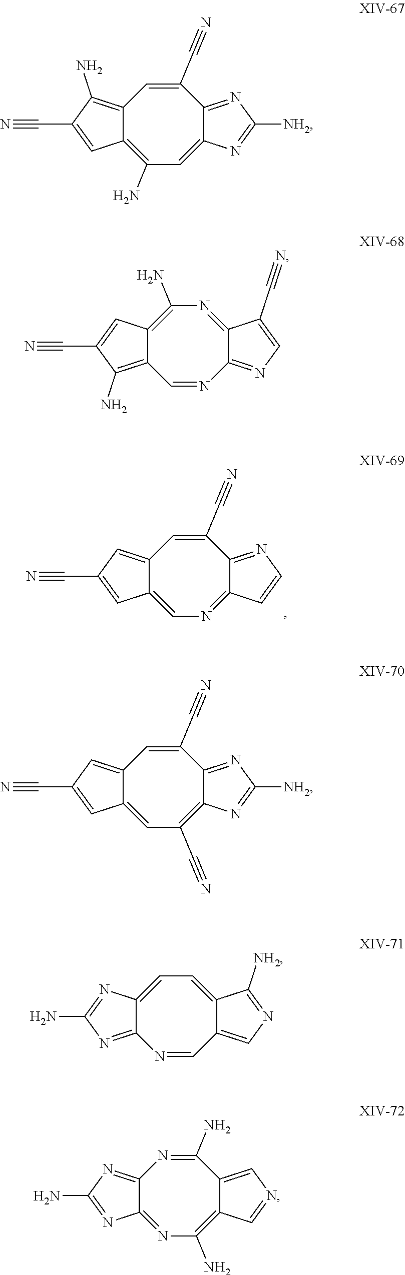 [SMILES: N#Cc1cc2c(N)c(C#N)cc-2c(N)cc2nc(N)nc1-2 ; N#Cc1cnc2ncc3c(N)c(C#N)cc-3c(N)nc1-2 ; N#Cc1cc2cnc3ccnc-3c(C#N)cc-2c1 ; N#Cc1cc2cc(C#N)c3nc(N)nc-3c(C#N)cc-2c1 ; Nc1nc2ccc3c(N)ncc-3cnc-2n1 ; Nc1nc2nc(N)c3cncc-3c(N)nc-2n1]

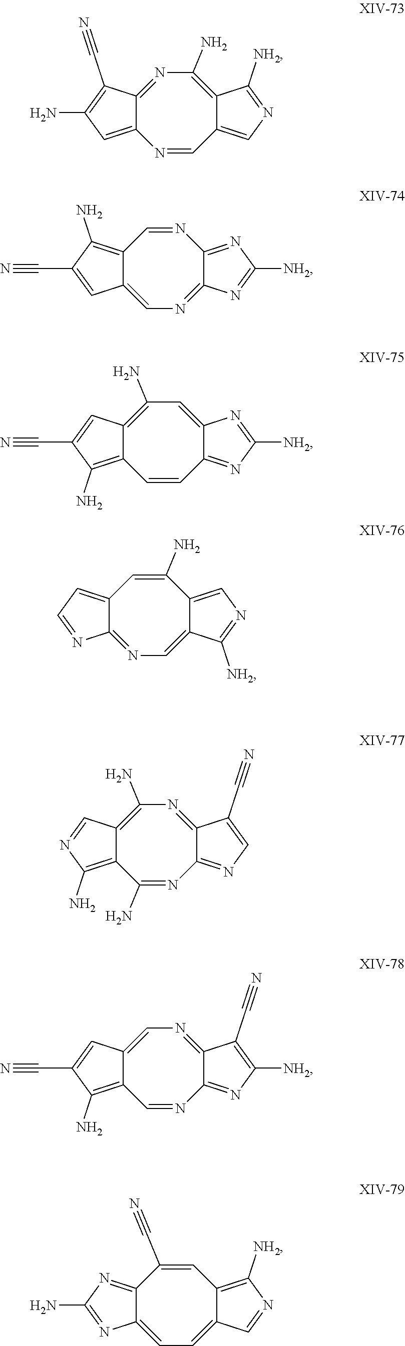

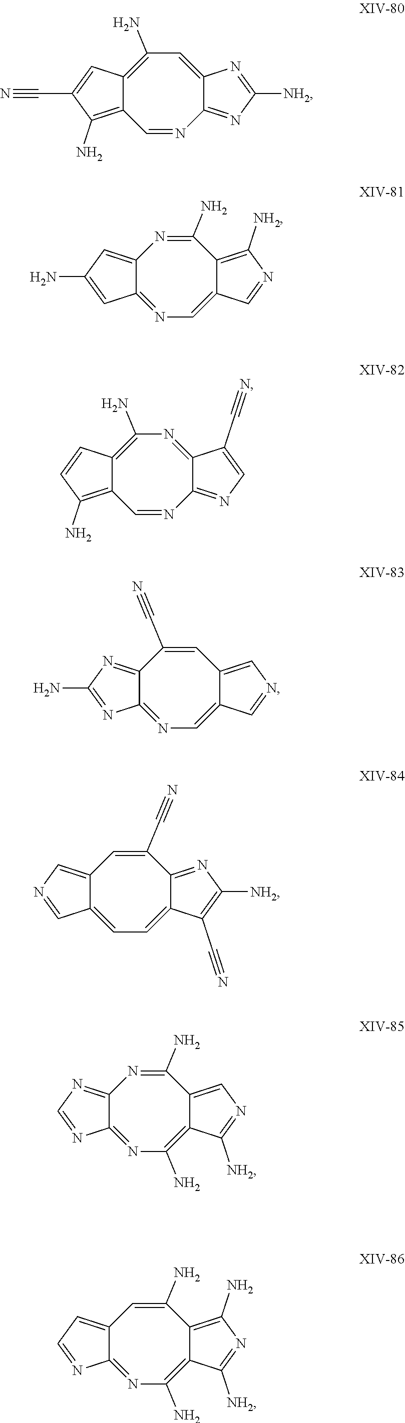

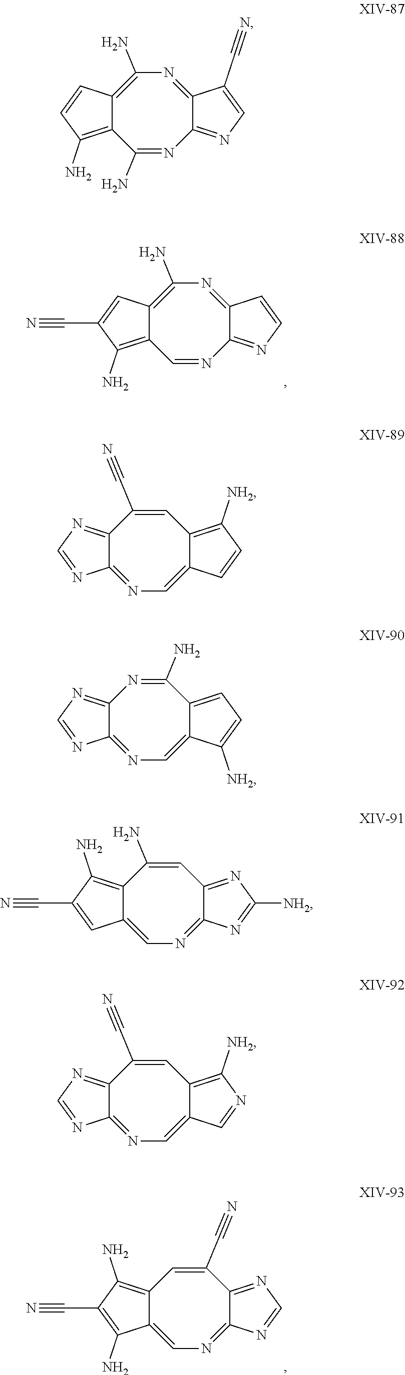

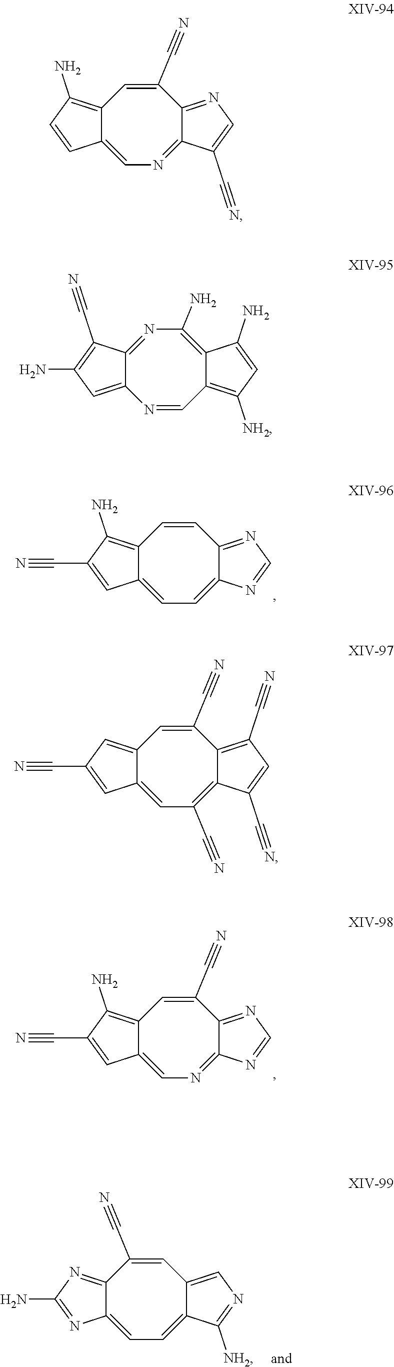 [SMILES: N#Cc1cnc2c(C#N)cc3c(N)ccc-3cnc1-2 ; N#Cc1c(N)cc2ncc3c(N)cc(N)c-3c(N)nc1-2 ; N#Cc1cc2ccc3ncnc-3ccc-2c1N ; N#Cc1cc2cc(C#N)c3c(C#N)cc(C#N)c-3c(C#N)cc-2c1 ; N#Cc1cc2c(N)c(C#N)cc-2cnc2ncnc1-2 ; N#Cc1cc2cnc(N)c-2ccc2nc(N)nc1-2]

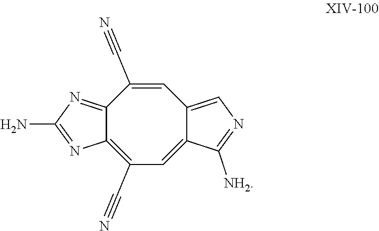 [SMILES: N#Cc1cc2cnc(N)c-2cc(C#N)c2nc(N)nc1-2]

Some exemplary compounds of Family XV are shown below:

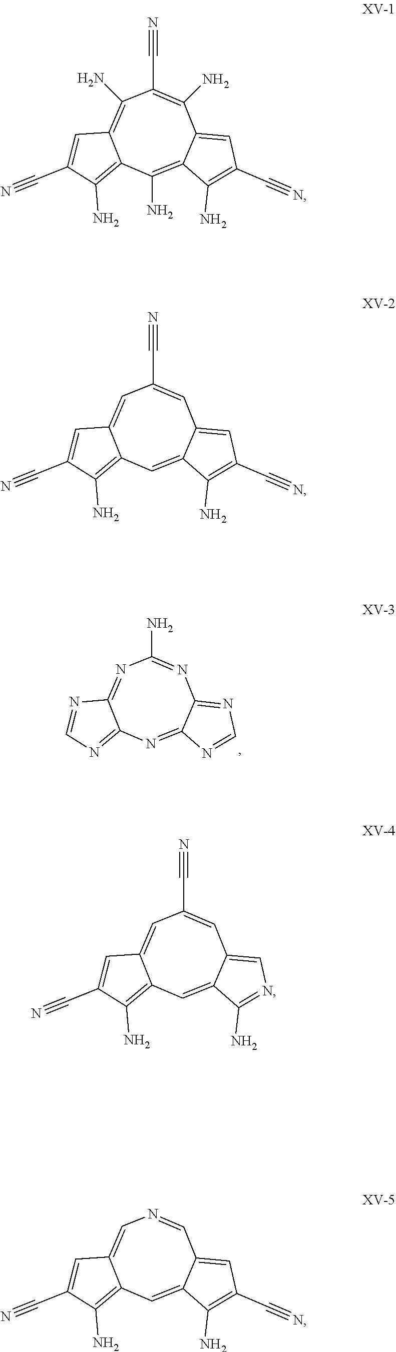 [SMILES: N#Cc1cc2c(N)c(C#N)c(N)c3cc(C#N)c(N)c-3c(N)c-2c1N ; N#Cc1cc2cc(C#N)c(N)c-2cc2c(N)c(C#N)cc-2c1 ; Nc1nc2ncnc-2nc2ncnc-2n1 ; N#Cc1cc2cnc(N)c-2cc2c(N)c(C#N)cc-2c1 ; N#Cc1cc2cncc3cc(C#N)c(N)c-3cc-2c1N]

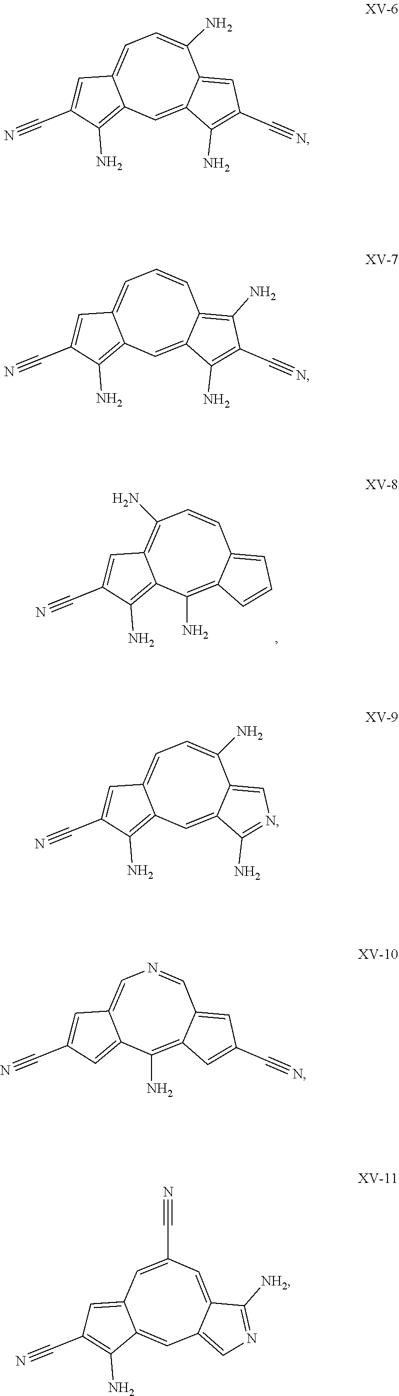 [SMILES: N#Cc1cc2ccc(N)c3cc(C#N)c(N)c-3cc-2c1N ; N#Cc1cc2cccc3c(N)c(C#N)c(N)c-3cc-2c1N ; N#Cc1cc2c(N)ccc3cccc-3c(N)c-2c1N ; N#Cc1cc2ccc(N)c3cnc(N)c-3cc-2c1N ; N#Cc1cc2cncc3cc(C#N)cc-3c(N)c-2c1 ; N#Cc1cc2cc(C#N)c(N)c-2cc2cnc(N)c-2c1]

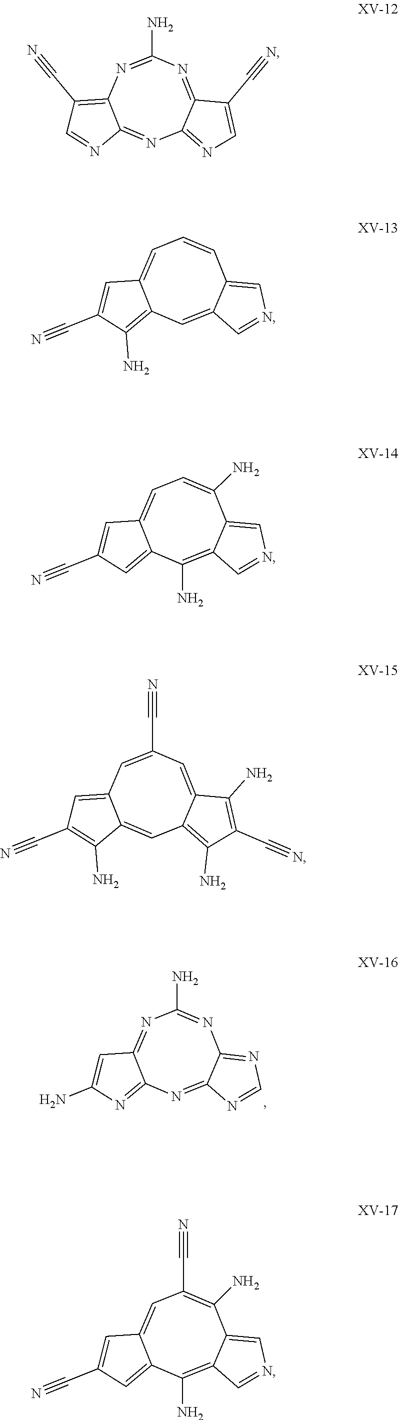 [SMILES: N#Cc1cnc2nc3ncc(C#N)c-3nc(N)nc1-2 ; N#Cc1cc2cccc3cncc-3cc-2c1N ; N#Cc1cc2ccc(N)c3cncc-3c(N)c-2c1 ; N#Cc1cc2cc(C#N)c(N)c-2cc2c(N)c(C#N)c(N)c-2c1 ; Nc1cc2nc(N)nc3ncnc-3nc-2n1 ; N#Cc1cc2cc(C#N)c(N)c3cncc-3c(N)c-2c1]

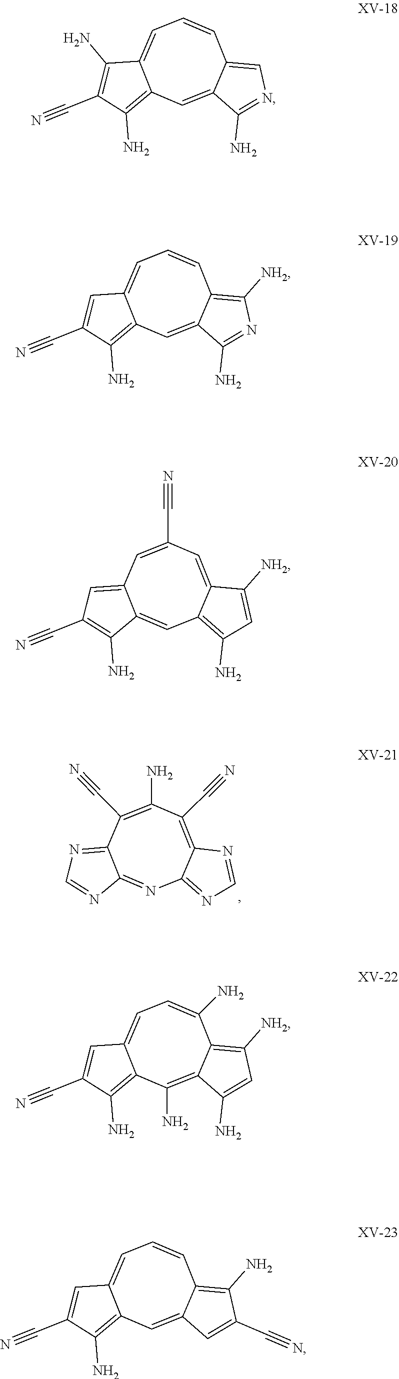 [SMILES: N#Cc1c(N)c2cccc3cnc(N)c-3cc-2c1N ; N#Cc1cc2cccc3c(N)nc(N)c-3cc-2c1N ; N#Cc1cc2cc(C#N)c(N)c-2cc2c(N)cc(N)c-2c1 ; N#Cc1c2ncnc-2nc2ncnc-2c(C#N)c1N ; N#Cc1cc2ccc(N)c3c(N)cc(N)c-3c(N)c-2c1N ; N#Cc1cc2cc3c(N)c(C#N)cc-3cccc-2c1N]

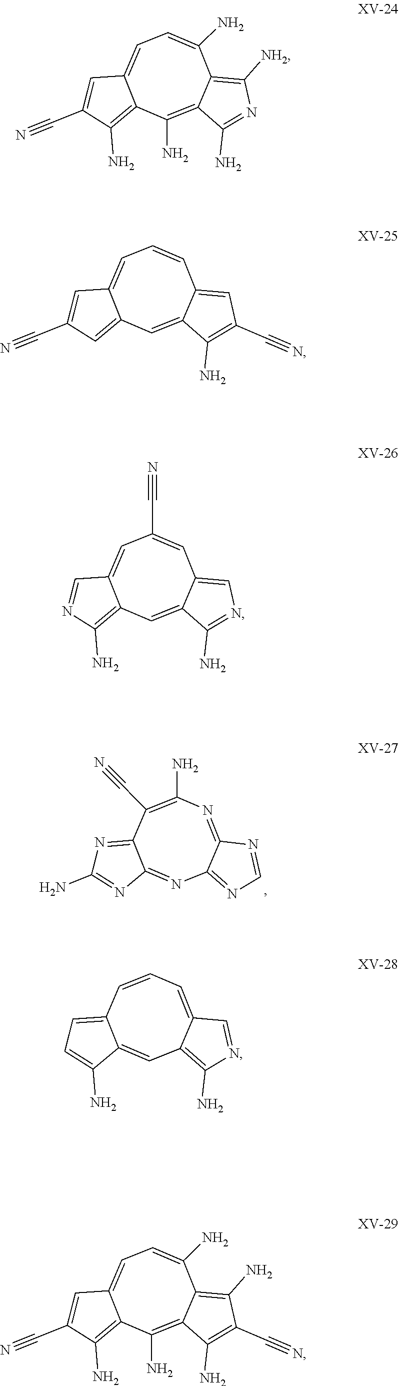 [SMILES: N#Cc1cc2ccc(N)c3c(N)nc(N)c-3c(N)c-2c1N ; N#Cc1cc2cccc3cc(C#N)c(N)c-3cc-2c1 ; N#Cc1cc2cnc(N)c-2cc2c(N)ncc-2c1 ; N#Cc1c(N)nc2ncnc-2nc2nc(N)nc1-2 ; Nc1ccc2cccc3cnc(N)c-3cc1-2 ; N#Cc1cc2ccc(N)c3c(N)c(C#N)c(N)c-3c(N)c-2c1N]

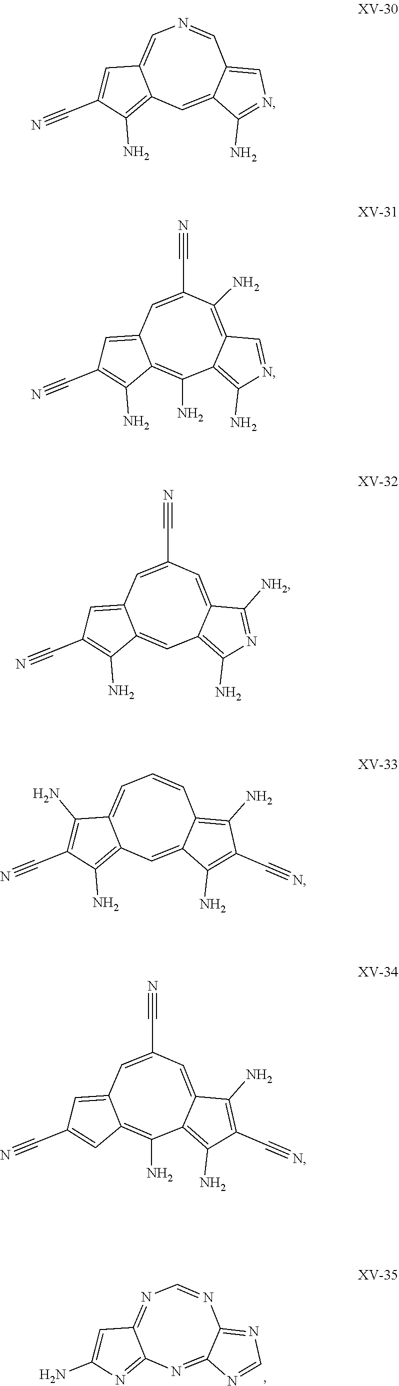 [SMILES: N#Cc1cc2cncc3cnc(N)c-3cc-2c1N ; N#Cc1cc2cc(C#N)c(N)c-2c(N)c2c(N)ncc-2c1N ; N#Cc1cc2cc(C#N)c(N)c-2cc2c(N)nc(N)c-2c1 ; N#Cc1c(N)c2cccc3c(N)c(C#N)c(N)c-3cc-2c1N ; N#Cc1cc2cc(C#N)cc-2c(N)c2c(N)c(C#N)c(N)c-2c1 ; Nc1cc2ncnc3ncnc-3nc-2n1]

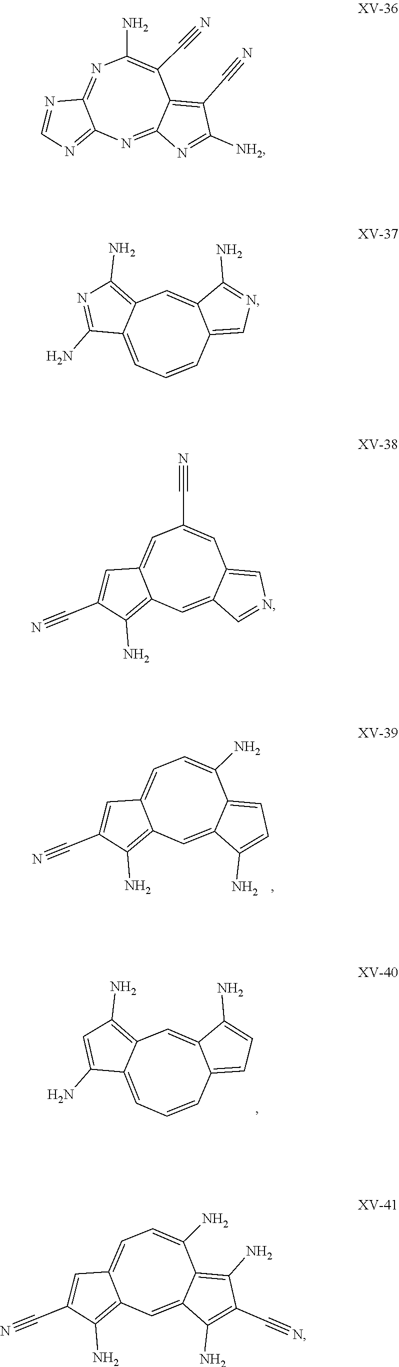 [SMILES: N#Cc1c(N)nc2ncnc-2nc2nc(N)c(C#N)c1-2 ; Nc1ncc2cccc3c(N)nc(N)c-3cc1-2 ; N#Cc1cc2cncc-2cc2c(N)c(C#N)cc-2c1 ; N#Cc1cc2ccc(N)c3ccc(N)c-3cc-2c1N ; Nc1ccc2cccc3c(N)cc(N)c-3cc1-2 ; N#Cc1cc2ccc(N)c3c(N)c(C#N)c(N)c-3cc-2c1N]

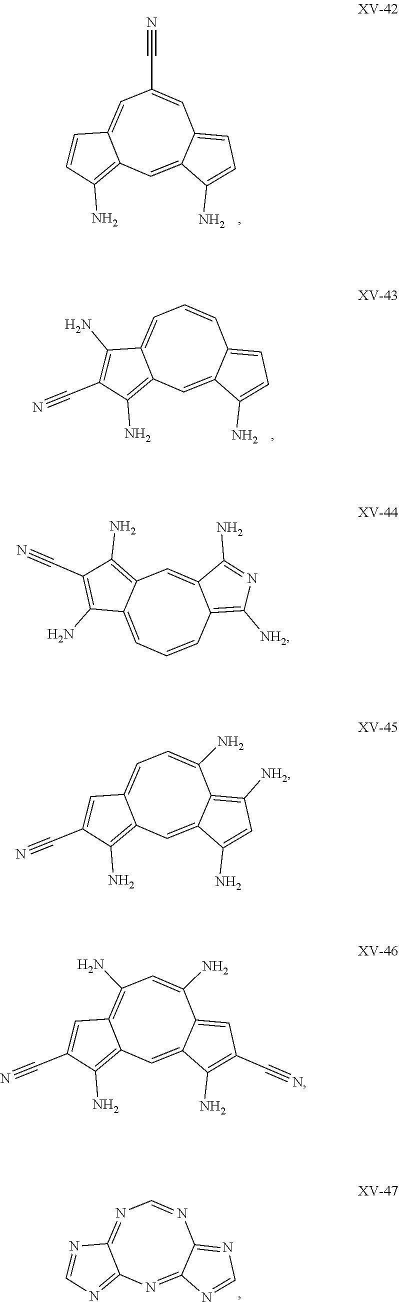 [SMILES: N#Cc1cc2ccc(N)c-2cc2c(N)ccc-2c1 ; N#Cc1c(N)c2cccc3ccc(N)c-3cc-2c1N ; N#Cc1c(N)c2cccc3c(N)nc(N)c-3cc-2c1N ; N#Cc1cc2ccc(N)c3c(N)cc(N)c-3cc-2c1N ; N#Cc1cc2c(N)cc(N)c3cc(C#N)c(N)c-3cc-2c1N ; c1nc2ncnc-2nc2ncnc-2n1]

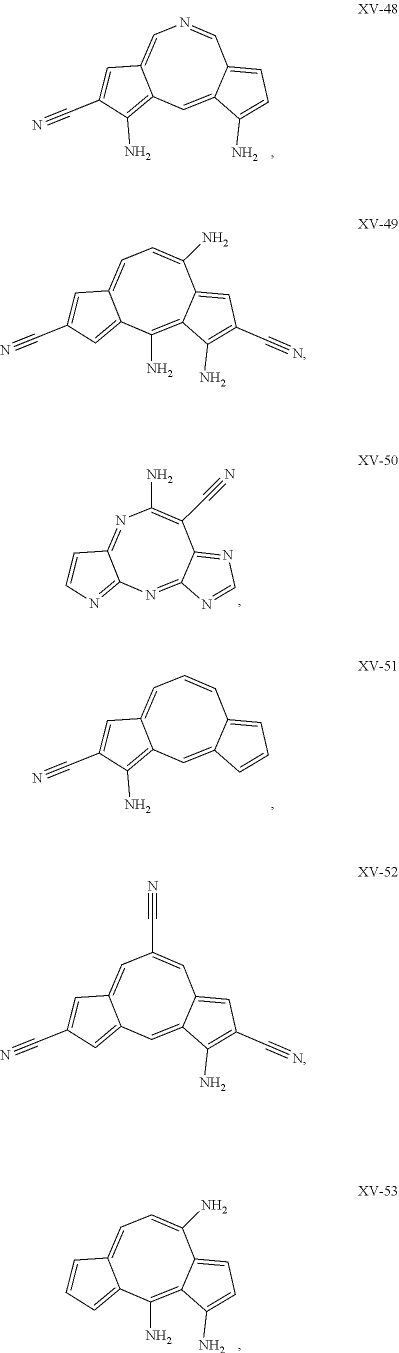 [SMILES: N#Cc1cc2cncc3ccc(N)c-3cc-2c1N ; N#Cc1cc2ccc(N)c3cc(C#N)c(N)c-3c(N)c-2c1 ; N#Cc1c(N)nc2ccnc-2nc2ncnc1-2 ; N#Cc1cc2cccc3cccc-3cc-2c1N ; N#Cc1cc2cc(C#N)cc3cc(C#N)c(N)c-3cc-2c1 ; Nc1ccc2cccc-2c(N)c2c(N)ccc1-2]

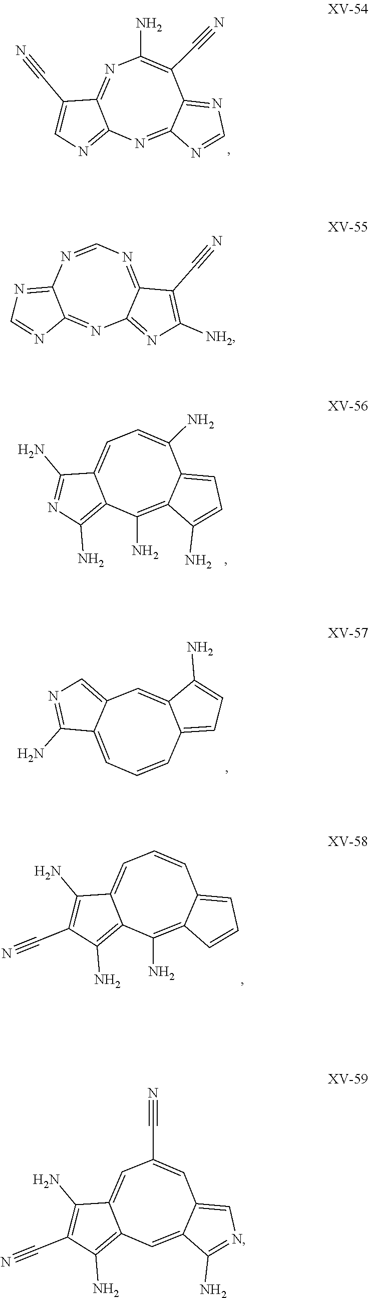 [SMILES: N#Cc1cnc2nc3ncnc-3c(C#N)c(N)nc1-2 ; N#Cc1c(N)nc2nc3ncnc-3ncnc1-2 ; Nc1ccc2c(N)nc(N)c-2c(N)c2c(N)ccc1-2 ; Nc1ccc2cccc3c(N)ncc-3cc1-2 ; N#Cc1c(N)c2cccc3cccc-3c(N)c-2c1N ; N#Cc1cc2cnc(N)c-2cc2c(N)c(C#N)c(N)c-2c1]

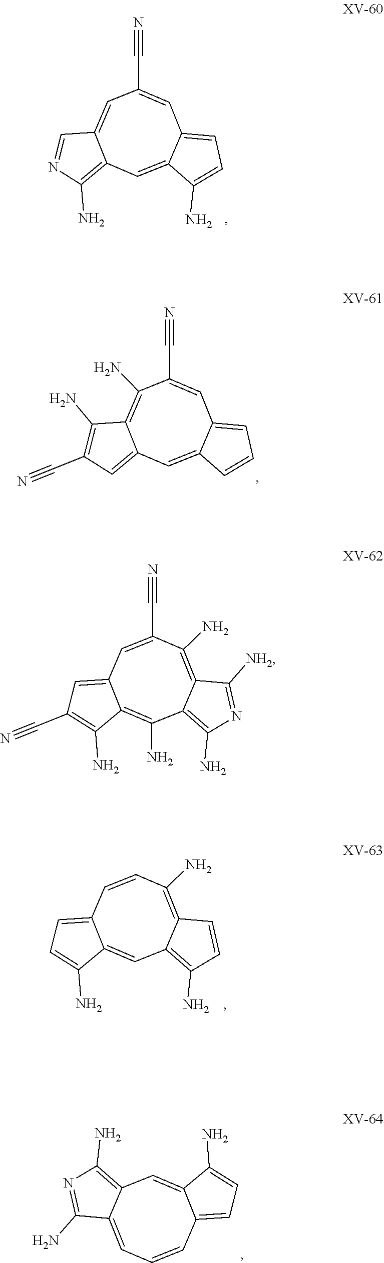 [SMILES: N#Cc1cc2ccc(N)c-2cc2c(N)ncc-2c1 ; N#Cc1cc2cccc-2cc2cc(C#N)c(N)c-2c1N ; N#Cc1cc2cc(C#N)c(N)c-2c(N)c2c(N)nc(N)c-2c1N ; Nc1ccc2ccc(N)c3ccc(N)c-3cc1-2 ; Nc1ccc2cccc3c(N)nc(N)c-3cc1-2]

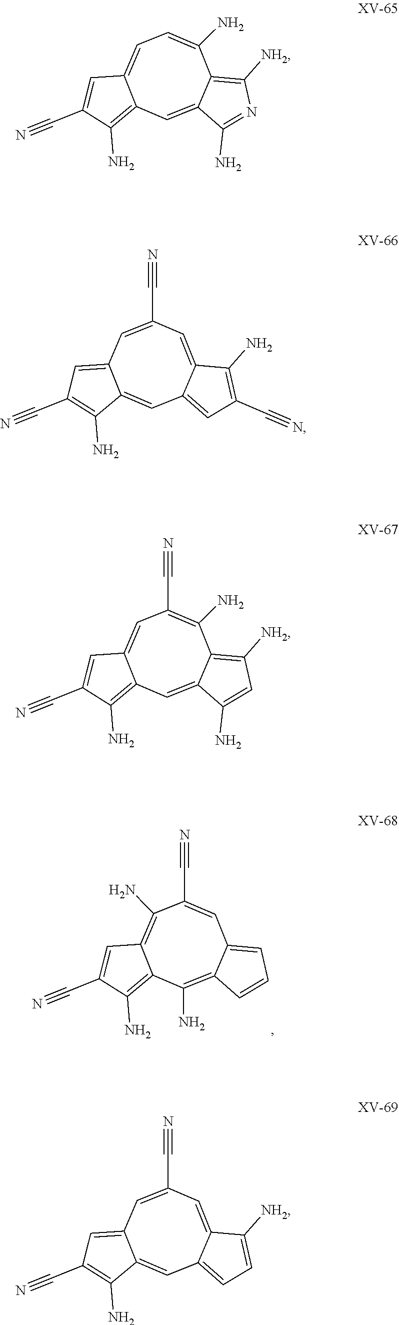 [SMILES: N#Cc1cc2ccc(N)c3c(N)nc(N)c-3cc-2c1N ; N#Cc1cc2cc(C#N)c(N)c-2cc2cc(C#N)c(N)c-2c1 ; N#Cc1cc2cc(C#N)c(N)c3c(N)cc(N)c-3cc-2c1N ; N#Cc1cc2cccc-2c(N)c2c(N)c(C#N)cc-2c1N ; N#Cc1cc2cc(C#N)c(N)c-2cc2ccc(N)c-2c1]

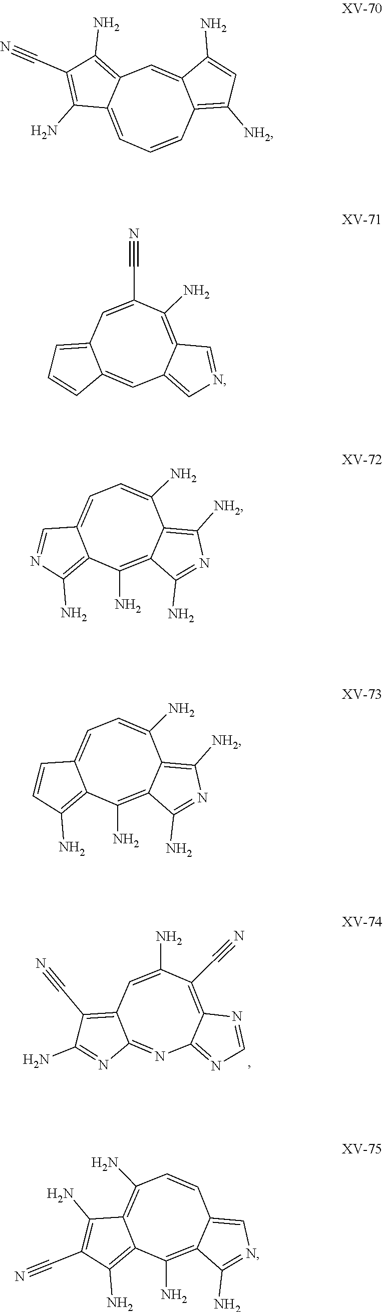 [SMILES: N#Cc1c(N)c2cccc3c(N)cc(N)c-3cc-2c1N ; N#Cc1cc2cccc-2cc2cncc-2c1N ; Nc1ccc2cnc(N)c-2c(N)c2c(N)nc(N)c1-2 ; Nc1ccc2ccc(N)c-2c(N)c2c(N)nc(N)c1-2 ; N#Cc1c2cc(N)c(C#N)c3ncnc-3nc-2nc1N ; N#Cc1c(N)c2c(N)ccc3cnc(N)c-3c(N)c-2c1N]

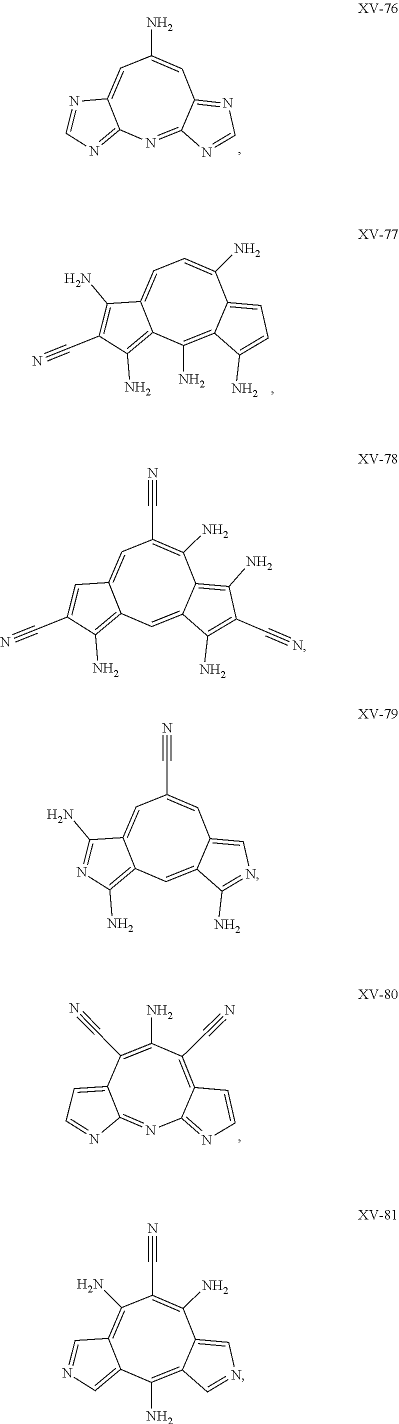 [SMILES: Nc1cc2ncnc-2nc2ncnc-2c1 ; N#Cc1c(N)c2ccc(N)c3ccc(N)c-3c(N)c-2c1N ; N#Cc1cc2cc(C#N)c(N)c3c(N)c(C#N)c(N)c-3cc-2c1N ; N#Cc1cc2cnc(N)c-2cc2c(N)nc(N)c-2c1 ; N#Cc1c2ccnc-2nc2nccc-2c(C#N)c1N ; N#Cc1c(N)c2cncc-2c(N)c2cncc-2c1N]

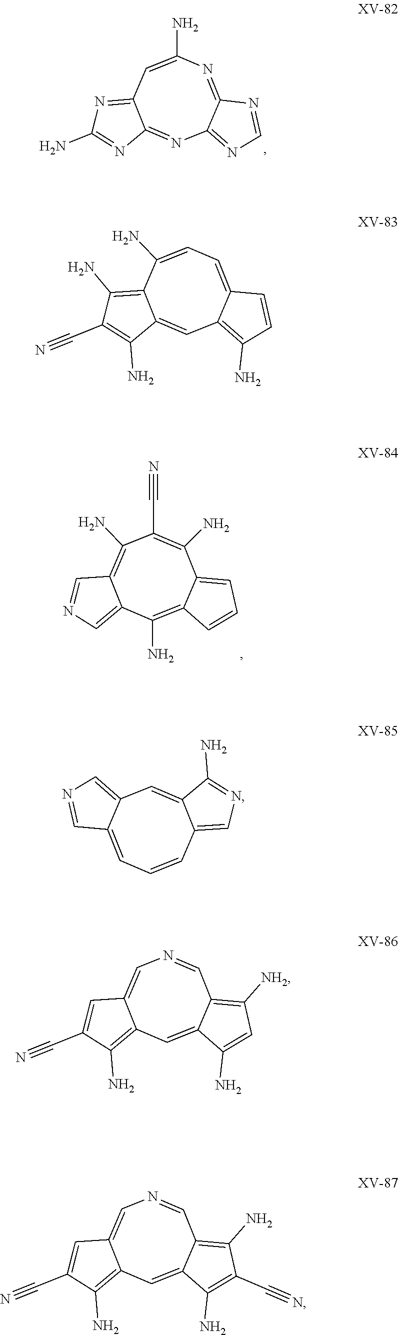 [SMILES: Nc1cc2nc(N)nc-2nc2ncnc-2n1 ; N#Cc1c(N)c2cc3c(N)ccc-3ccc(N)c-2c1N ; N#Cc1c(N)c2cccc-2c(N)c2cncc-2c1N ; Nc1ncc2cccc3cncc-3cc1-2 ; N#Cc1cc2cncc3c(N)cc(N)c-3cc-2c1N ; N#Cc1cc2cncc3c(N)c(C#N)c(N)c-3cc-2c1N]

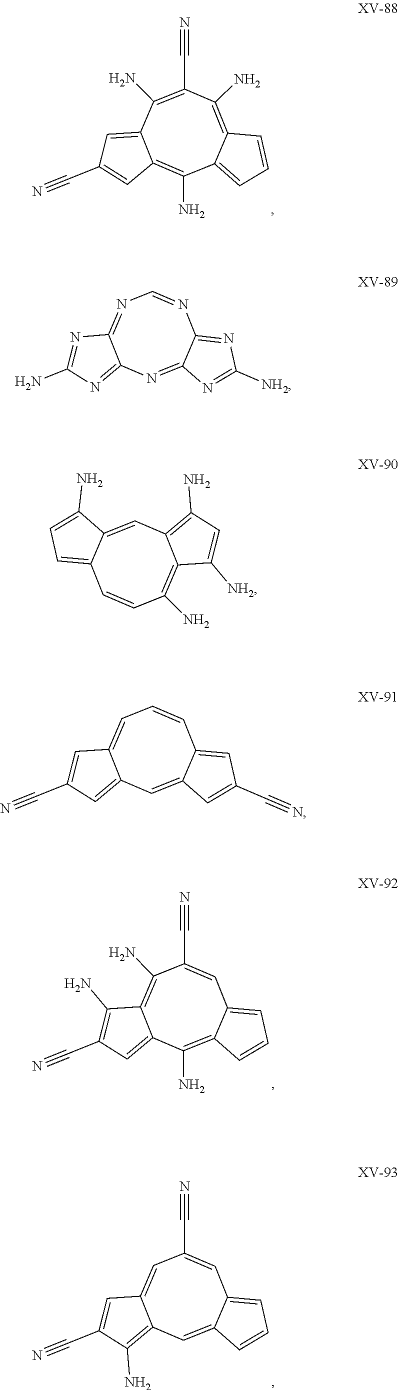 [SMILES: N#Cc1cc2c(N)c3cccc-3c(N)c(C#N)c(N)c-2c1 ; Nc1nc2ncnc3nc(N)nc-3nc-2n1 ; Nc1ccc2ccc(N)c3c(N)cc(N)c-3cc1-2 ; N#Cc1cc2cccc3cc(C#N)cc-3cc-2c1 ; N#Cc1cc2cccc-2c(N)c2cc(C#N)c(N)c-2c1N ; N#Cc1cc2cccc-2cc2c(N)c(C#N)cc-2c1]

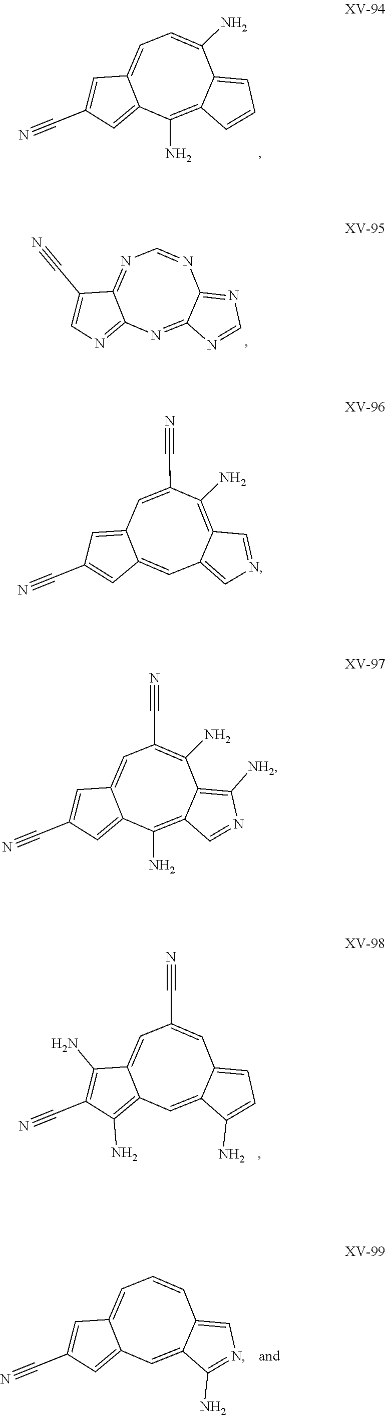 [SMILES: N#Cc1cc2ccc(N)c3cccc-3c(N)c-2c1 ; N#Cc1cnc2nc3ncnc-3ncnc1-2 ; N#Cc1cc2cc3cncc-3c(N)c(C#N)cc-2c1 ; N#Cc1cc2cc(C#N)c(N)c3c(N)ncc-3c(N)c-2c1 ; N#Cc1cc2ccc(N)c-2cc2c(N)c(C#N)c(N)c-2c1 ; N#Cc1cc2cccc3cnc(N)c-3cc-2c1]

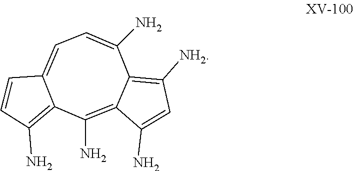 [SMILES: Nc1ccc2ccc(N)c-2c(N)c2c(N)cc(N)c1-2]

Some exemplary compounds of Family XVI are shown below:

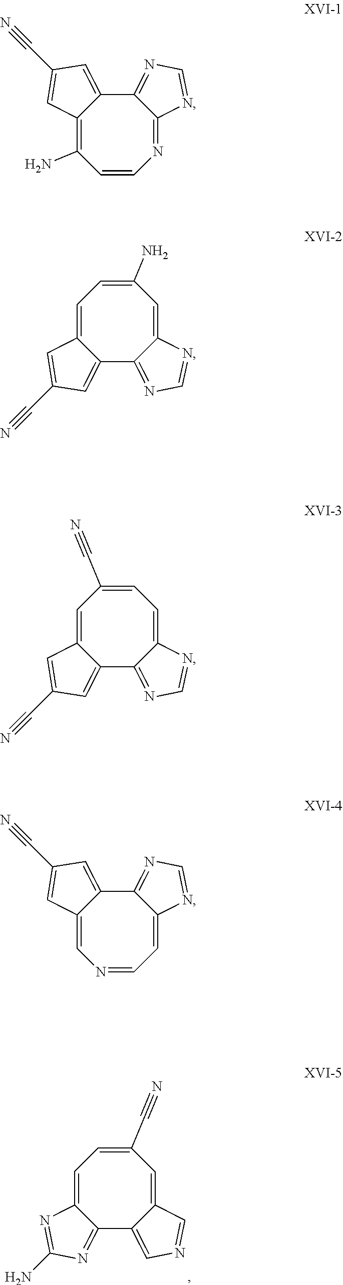 [SMILES: N#Cc1cc2c(N)ccnc3ncnc-3c-2c1 ; N#Cc1cc2ccc(N)cc3ncnc-3c-2c1 ; N#Cc1ccc2ncnc-2c2cc(C#N)cc-2c1 ; N#Cc1cc2cnccc3ncnc-3c-2c1 ; N#Cc1ccc2nc(N)nc-2c2cncc-2c1]

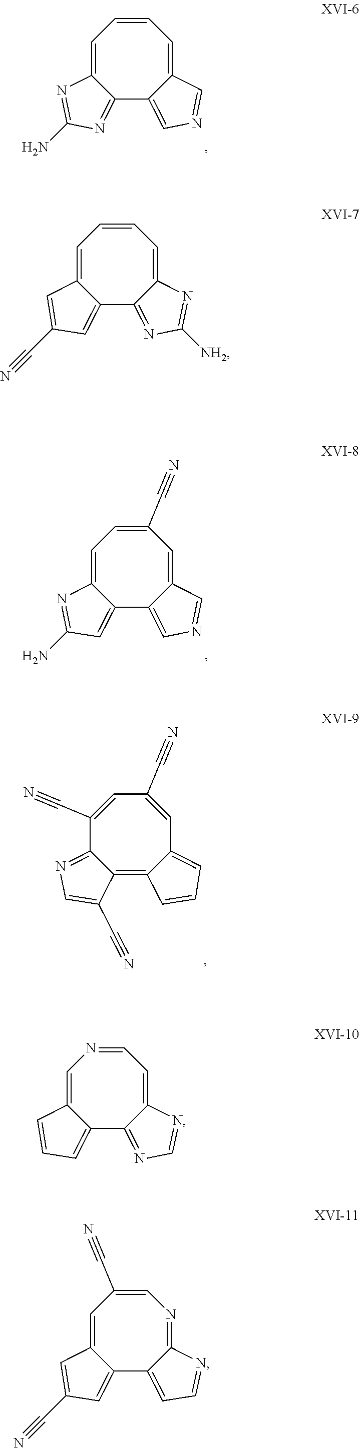 [SMILES: Nc1nc2ccccc3cncc-3c-2n1 ; N#Cc1cc2ccccc3nc(N)nc-3c-2c1 ; N#Cc1ccc2nc(N)cc-2c2cncc-2c1 ; N#Cc1cc2cccc-2c2c(C#N)cnc-2c(C#N)c1 ; c1cc2cnccc3ncnc-3c-2c1 ; N#Cc1cnc2nccc-2c2cc(C#N)cc-2c1]

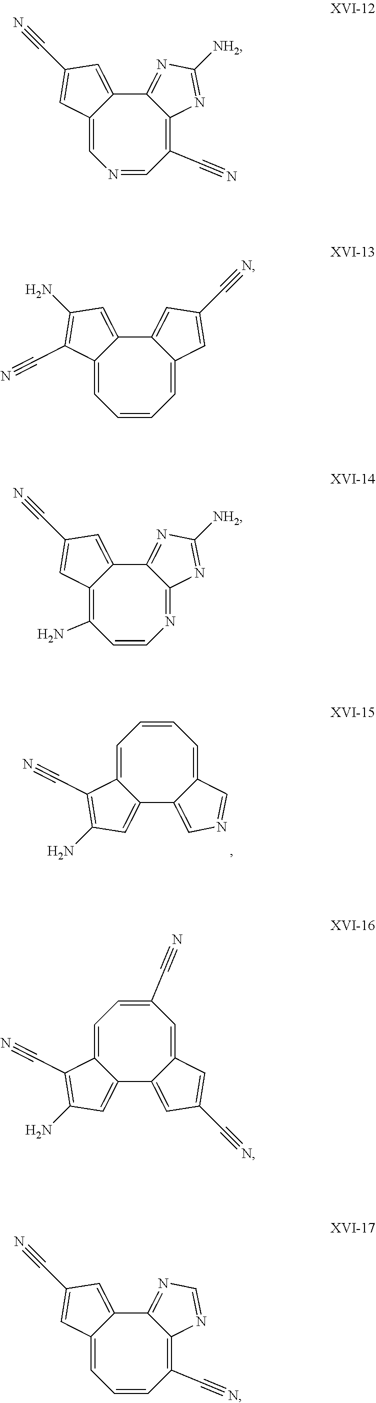 [SMILES: N#Cc1cc2cncc(C#N)c3nc(N)nc-3c-2c1 ; N#Cc1cc2ccccc3c(C#N)c(N)cc-3c-2c1 ; N#Cc1cc2c(N)ccnc3nc(N)nc-3c-2c1 ; N#Cc1c(N)cc2c3cncc-3ccccc1-2 ; N#Cc1ccc2c(C#N)c(N)cc-2c2cc(C#N)cc-2c1 ; N#Cc1cc2cccc(C#N)c3ncnc-3c-2c1]

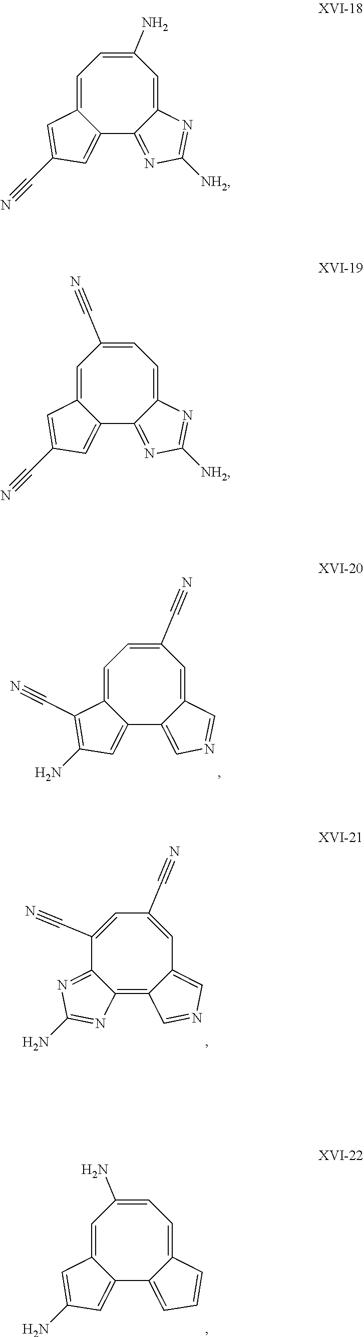 [SMILES: N#Cc1cc2ccc(N)cc3nc(N)nc-3c-2c1 ; N#Cc1ccc2nc(N)nc-2c2cc(C#N)cc-2c1 ; N#Cc1ccc2c(C#N)c(N)cc-2c2cncc-2c1 ; N#Cc1cc2cncc-2c2nc(N)nc-2c(C#N)c1 ; Nc1ccc2cccc-2c2cc(N)cc-2c1]

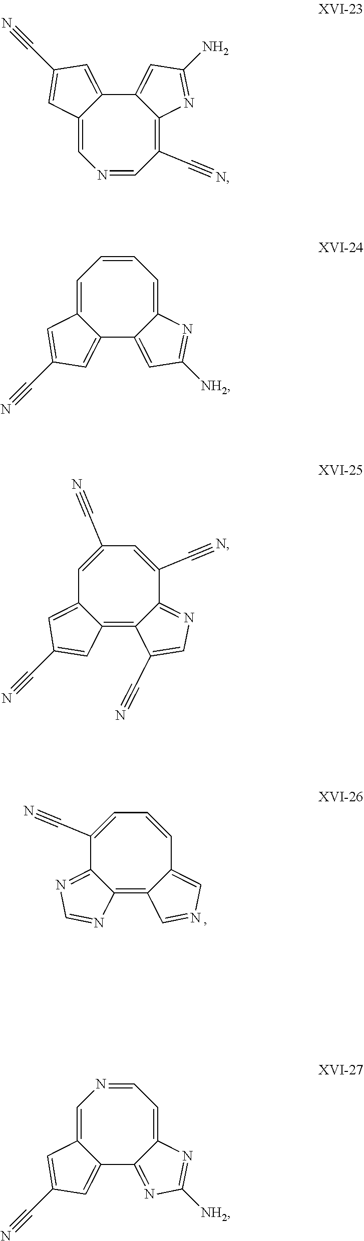 [SMILES: N#Cc1cc2cncc(C#N)c3nc(N)cc-3c-2c1 ; N#Cc1cc2ccccc3nc(N)cc-3c-2c1 ; N#Cc1cc2cc(C#N)cc-2c2c(C#N)cnc-2c(C#N)c1 ; N#Cc1cccc2cncc-2c2ncnc1-2 ; N#Cc1cc2cnccc3nc(N)nc-3c-2c1]

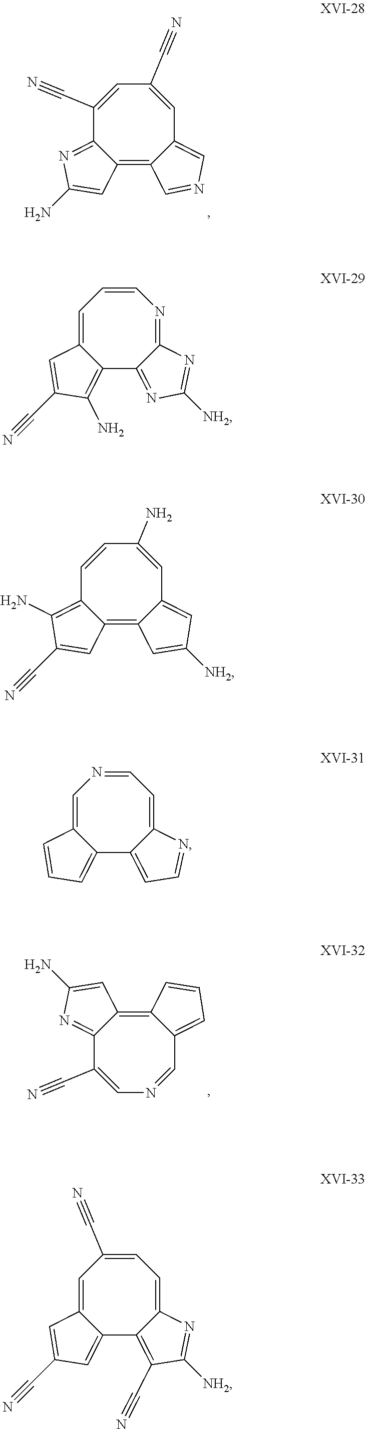 [SMILES: N#Cc1cc2cncc-2c2cc(N)nc-2c(C#N)c1 ; N#Cc1cc2cccnc3nc(N)nc-3c-2c1N ; N#Cc1cc2c3cc(N)cc-3cc(N)ccc-2c1N ; c1cc2cnccc3nccc-3c-2c1 ; N#Cc1cncc2cccc-2c2cc(N)nc1-2 ; N#Cc1ccc2nc(N)c(C#N)c-2c2cc(C#N)cc-2c1]

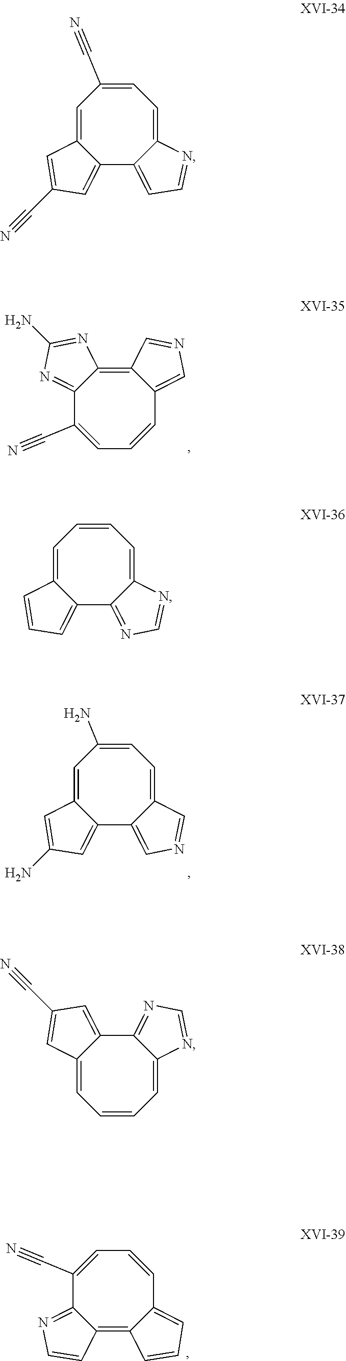 [SMILES: N#Cc1ccc2nccc-2c2cc(C#N)cc-2c1 ; N#Cc1cccc2cncc-2c2nc(N)nc1-2 ; c1ccc2ncnc-2c2cccc-2c1 ; Nc1ccc2cncc-2c2cc(N)cc-2c1 ; N#Cc1cc2ccccc3ncnc-3c-2c1 ; N#Cc1cccc2cccc-2c2ccnc1-2]

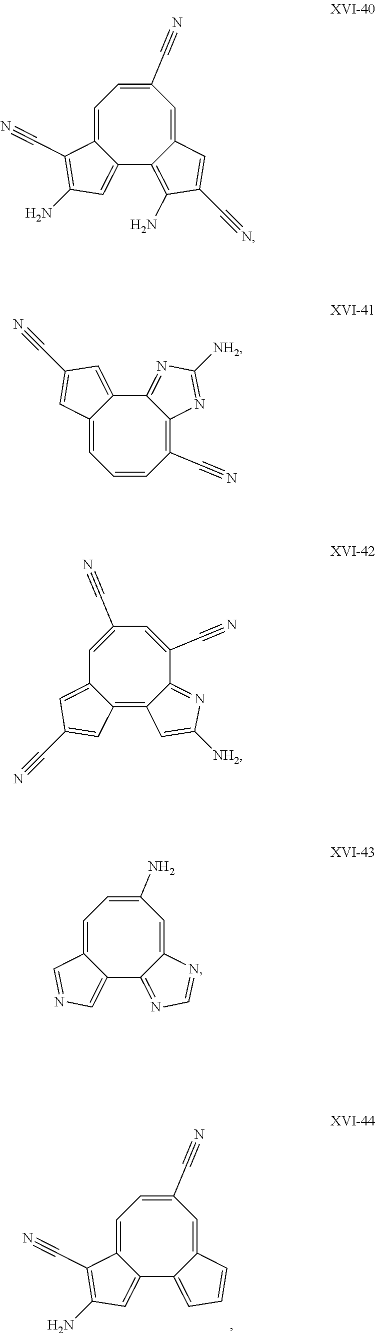 [SMILES: N#Cc1ccc2c(C#N)c(N)cc-2c2c(N)c(C#N)cc-2c1 ; N#Cc1cc2cccc(C#N)c3nc(N)nc-3c-2c1 ; N#Cc1cc2cc(C#N)cc-2c2cc(N)nc-2c(C#N)c1 ; Nc1ccc2cncc-2c2ncnc-2c1 ; N#Cc1ccc2c(C#N)c(N)cc-2c2cccc-2c1]

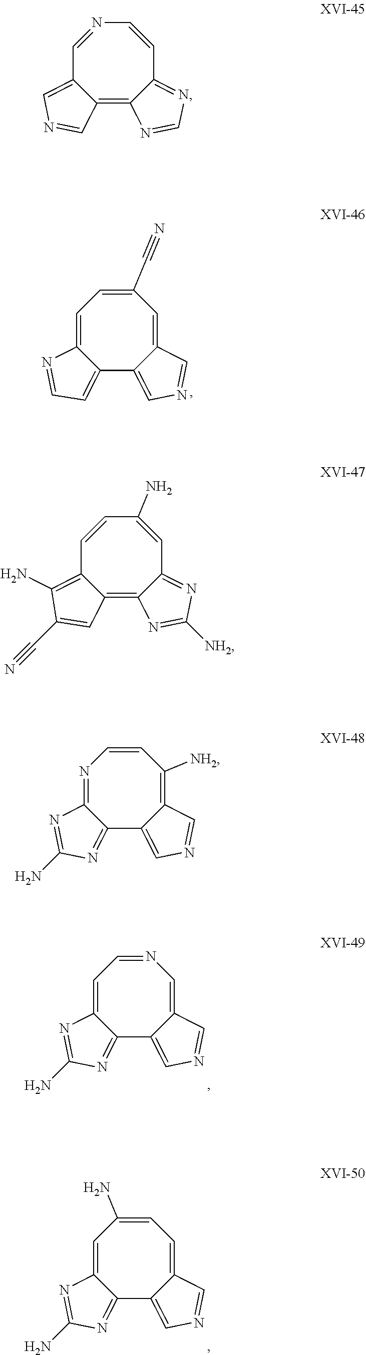 [SMILES: c1cc2ncnc-2c2cncc-2cn1 ; N#Cc1ccc2nccc-2c2cncc-2c1 ; N#Cc1cc2c3nc(N)nc-3cc(N)ccc-2c1N ; Nc1nc2nccc(N)c3cncc-3c-2n1 ; Nc1nc2ccncc3cncc-3c-2n1 ; Nc1ccc2cncc-2c2nc(N)nc-2c1]

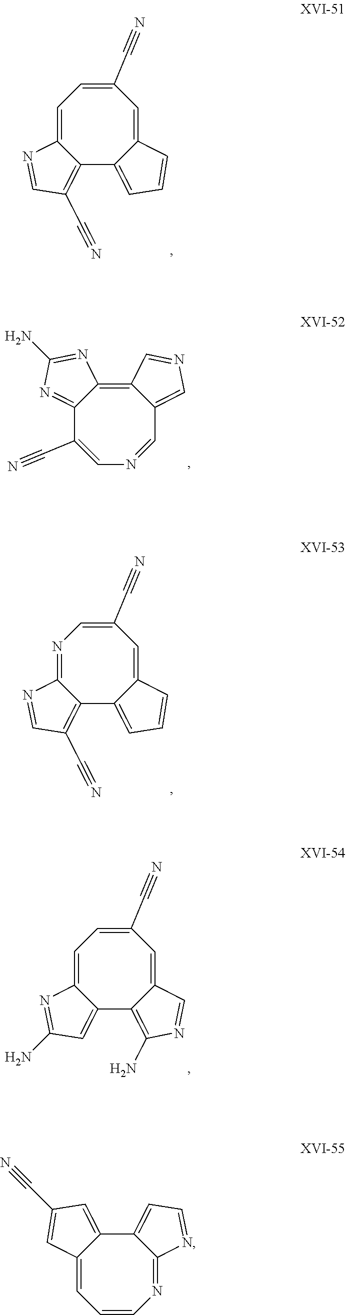 [SMILES: N#Cc1ccc2ncc(C#N)c-2c2cccc-2c1 ; N#Cc1cncc2cncc-2c2nc(N)nc1-2 ; N#Cc1cnc2ncc(C#N)c-2c2cccc-2c1 ; N#Cc1ccc2nc(N)cc-2c2c(N)ncc-2c1 ; N#Cc1cc2cccnc3nccc-3c-2c1]

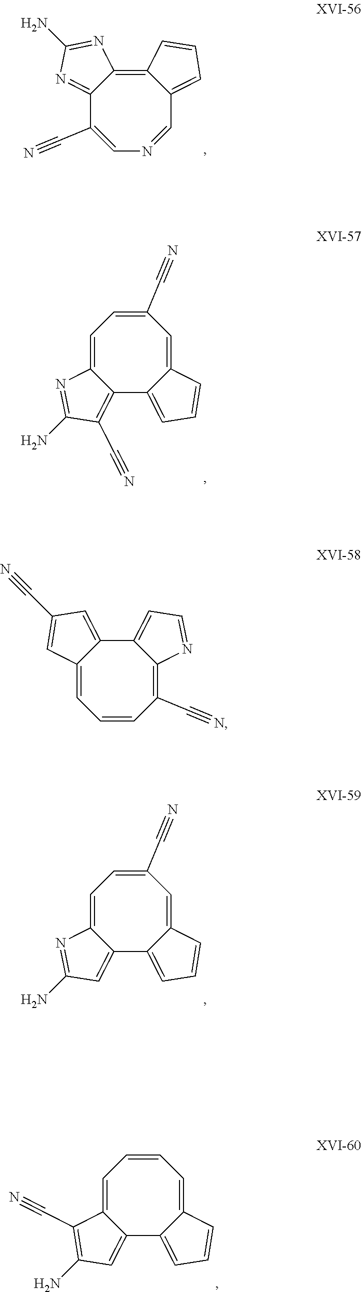 [SMILES: N#Cc1cncc2cccc-2c2nc(N)nc1-2 ; N#Cc1ccc2nc(N)c(C#N)c-2c2cccc-2c1 ; N#Cc1cc2cccc(C#N)c3nccc-3c-2c1 ; N#Cc1ccc2nc(N)cc-2c2cccc-2c1 ; N#Cc1c(N)cc2c3cccc-3ccccc1-2]

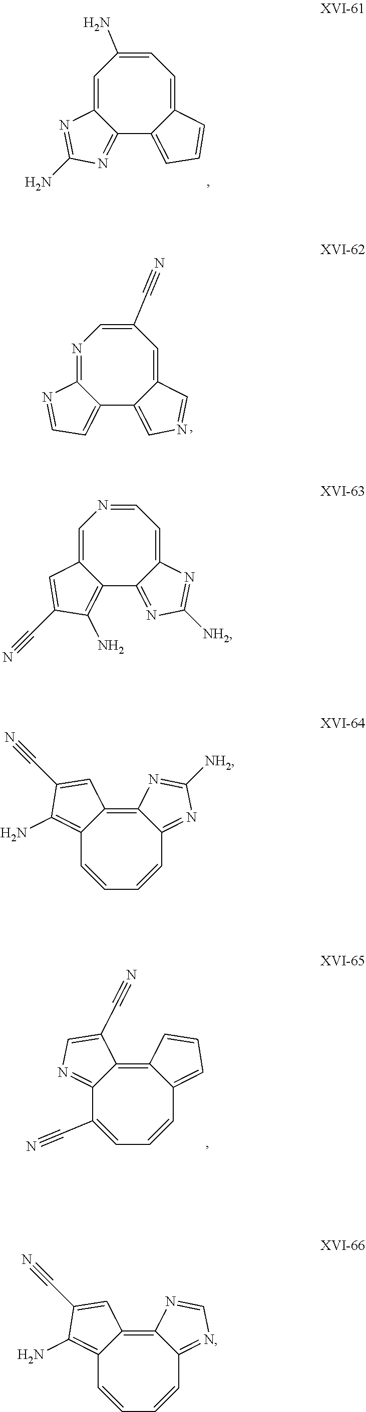 [SMILES: Nc1ccc2cccc-2c2nc(N)nc-2c1 ; N#Cc1cnc2nccc-2c2cncc-2c1 ; N#Cc1cc2cnccc3nc(N)nc-3c-2c1N ; N#Cc1cc2c3nc(N)nc-3ccccc-2c1N ; N#Cc1cccc2cccc-2c2c(C#N)cnc1-2 ; N#Cc1cc2c3ncnc-3ccccc-2c1N]

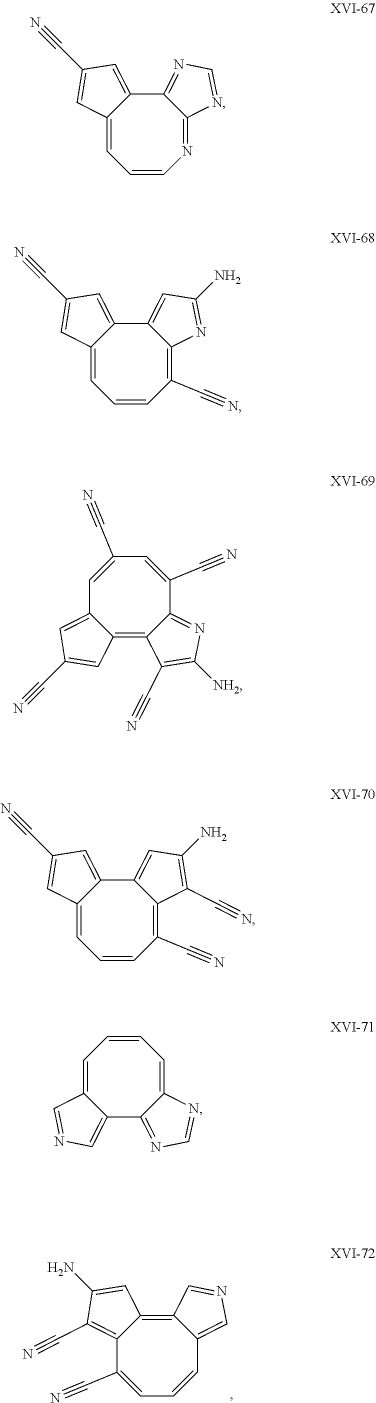 [SMILES: N#Cc1cc2cccnc3ncnc-3c-2c1 ; N#Cc1cc2cccc(C#N)c3nc(N)cc-3c-2c1 ; N#Cc1cc2cc(C#N)cc-2c2c(C#N)c(N)nc-2c(C#N)c1 ; N#Cc1cc2cccc(C#N)c3c(C#N)c(N)cc-3c-2c1 ; c1ccc2ncnc-2c2cncc-2c1 ; N#Cc1cccc2cncc-2c2cc(N)c(C#N)c1-2]

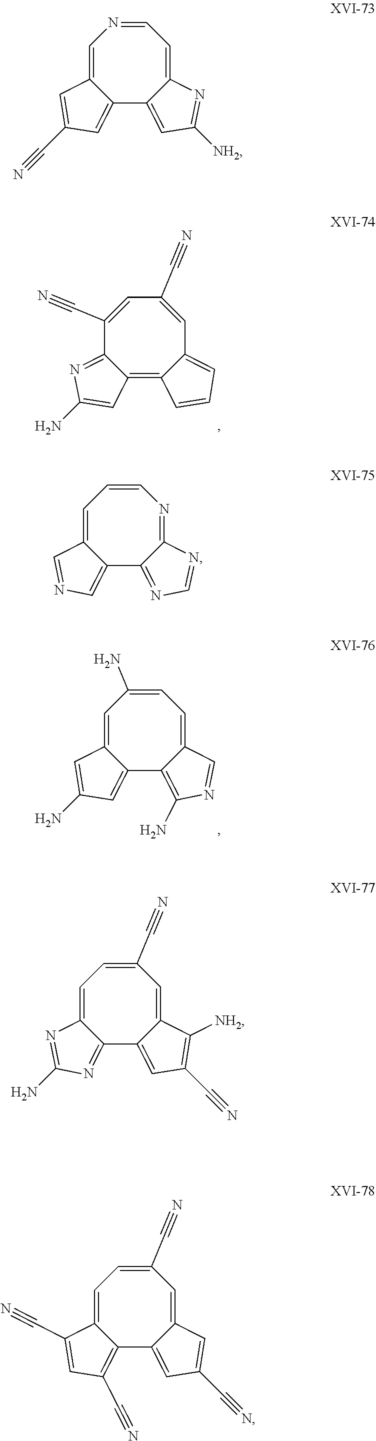 [SMILES: N#Cc1cc2cnccc3nc(N)cc-3c-2c1 ; N#Cc1cc2cccc-2c2cc(N)nc-2c(C#N)c1 ; c1cnc2ncnc-2c2cncc-2c1 ; Nc1ccc2cnc(N)c-2c2cc(N)cc-2c1 ; N#Cc1ccc2nc(N)nc-2c2cc(C#N)c(N)c-2c1 ; N#Cc1ccc2c(C#N)cc(C#N)c-2c2cc(C#N)cc-2c1]

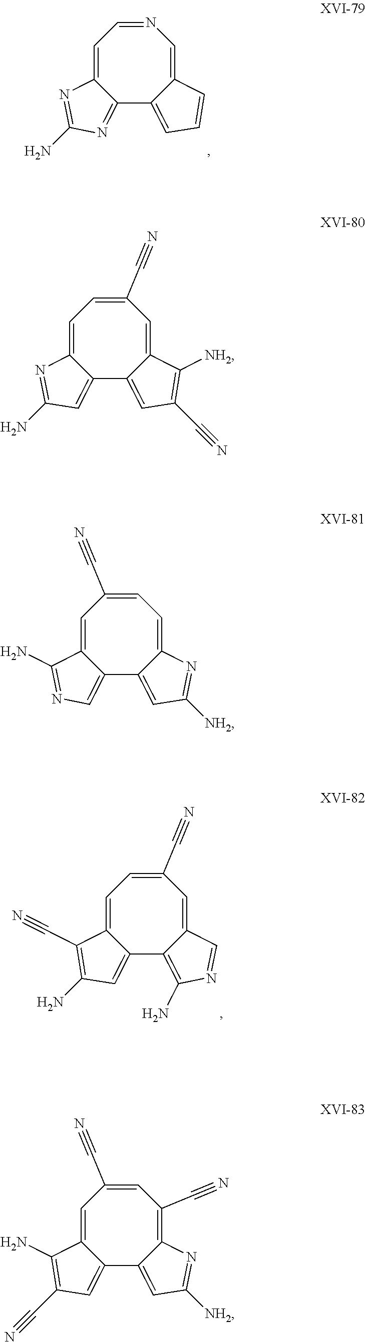 [SMILES: Nc1nc2ccncc3cccc-3c-2n1 ; N#Cc1ccc2nc(N)cc-2c2cc(C#N)c(N)c-2c1 ; N#Cc1ccc2nc(N)cc-2c2cnc(N)c-2c1 ; N#Cc1ccc2c(C#N)c(N)cc-2c2c(N)ncc-2c1 ; N#Cc1cc(C#N)c2nc(N)cc-2c2cc(C#N)c(N)c-2c1]

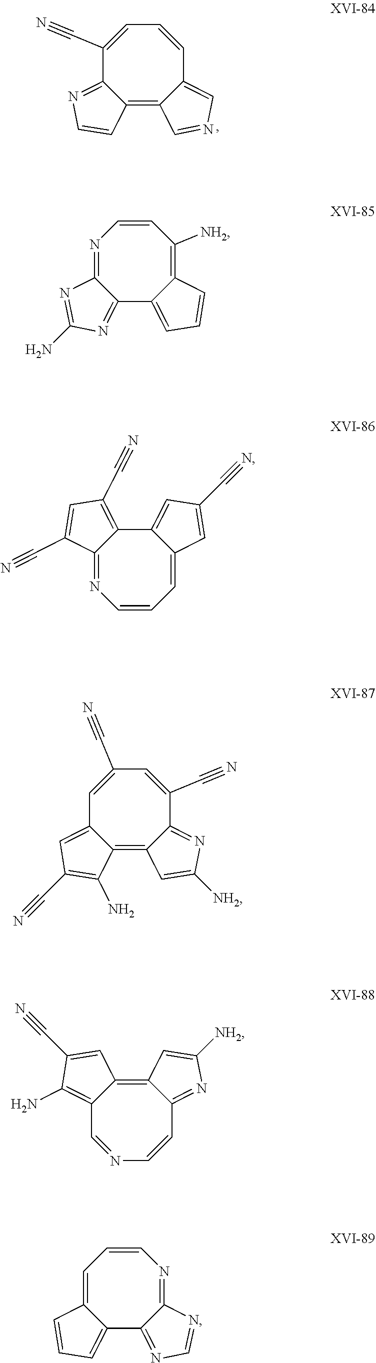 [SMILES: N#Cc1cccc2cncc-2c2ccnc1-2 ; Nc1nc2nccc(N)c3cccc-3c-2n1 ; N#Cc1cc2cccnc3c(C#N)cc(C#N)c-3c-2c1 ; N#Cc1cc2cc(C#N)c(N)c-2c2cc(N)nc-2c(C#N)c1 ; N#Cc1cc2c3cc(N)nc-3ccncc-2c1N ; c1cnc2ncnc-2c2cccc-2c1]

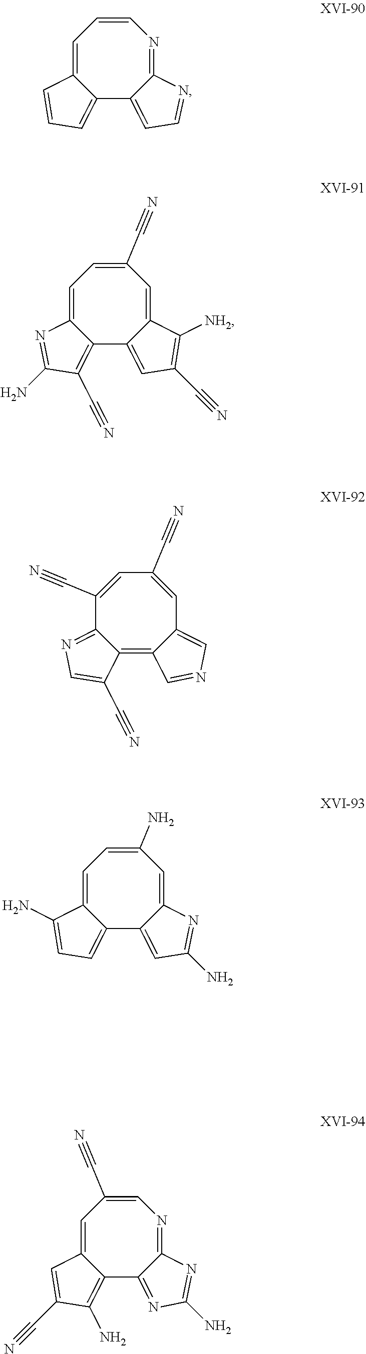 [SMILES: c1cnc2nccc-2c2cccc-2c1 ; N#Cc1ccc2nc(N)c(C#N)c-2c2cc(C#N)c(N)c-2c1 ; N#Cc1cc2cncc-2c2c(C#N)cnc-2c(C#N)c1 ; Nc1ccc2c(N)ccc-2c2cc(N)nc-2c1 ; N#Cc1cnc2nc(N)nc-2c2c(N)c(C#N)cc-2c1]

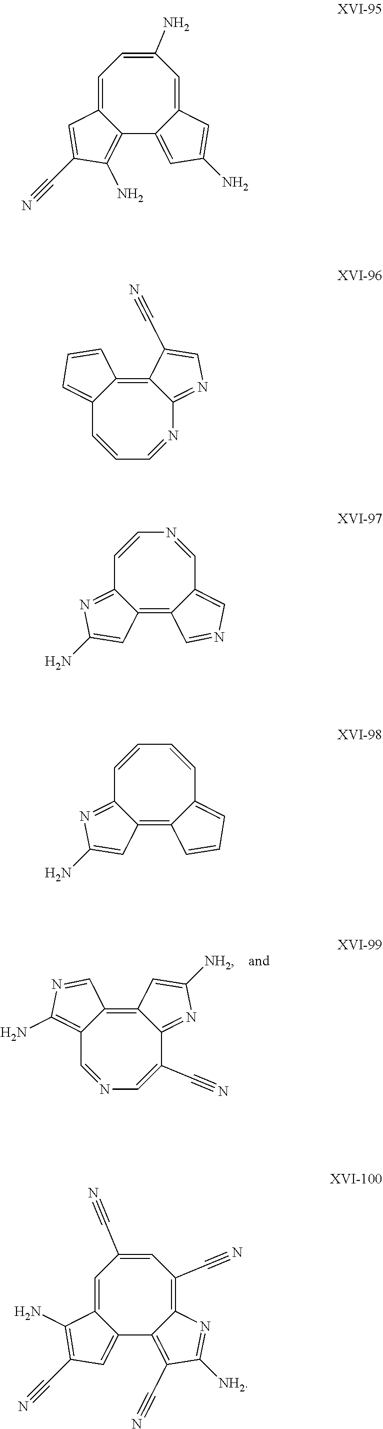 [SMILES: N#Cc1cc2ccc(N)cc3cc(N)cc-3c-2c1N ; N#Cc1cnc2ncccc3cccc-3c1-2 ; Nc1cc2c3cncc-3cnccc-2n1 ; Nc1cc2c3cccc-3ccccc-2n1 ; N#Cc1cncc2c(N)ncc-2c2cc(N)nc1-2 ; N#Cc1cc(C#N)c2nc(N)c(C#N)c-2c2cc(C#N)c(N)c-2c1]

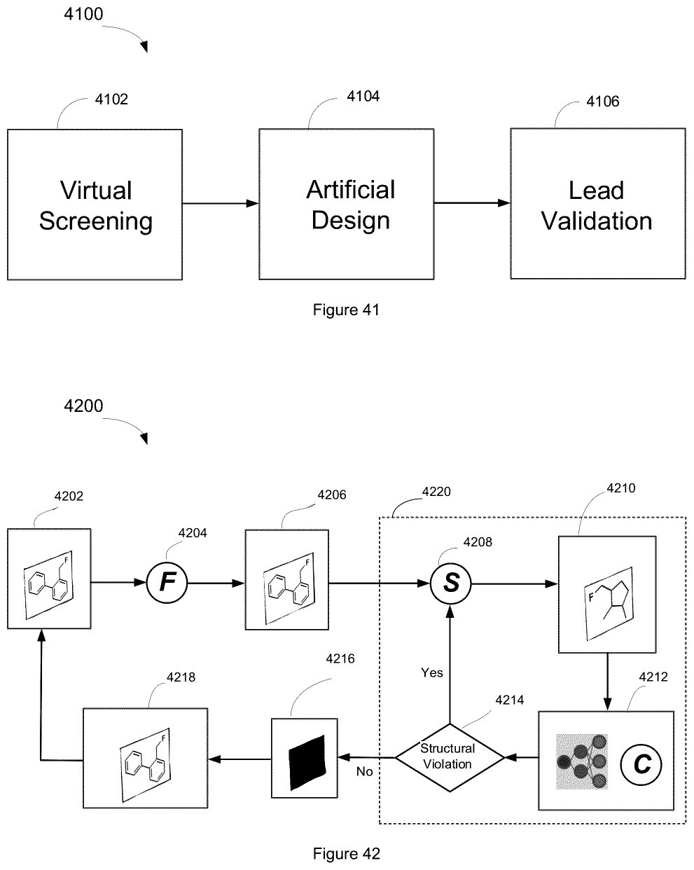

Reference is next made to FIGS. 41 and 42, which illustrate the use of an artificial molecular design workflow and genetic algorithm (GA) to identify a family of organic emitters with inverted singlet-triplet gaps and appreciable fluorescence rates. The genetic algorithm disclosed here can be used to generate at least some of the molecules discussed above or in combination with a bottom-up approach as disclosed herein. In one example, the use of the genetic algorithm enabled the exploration of more than 800,000 potential emitter molecules. It also resulted in identification of more than 10,000 candidates predicted to have inverted singlet-10 triplet gaps and appreciable fluorescence rates, many of which likely emit blue light, and uncovering of at least more than 1,000 promising structures.

FIG. 41 illustrates a flowchart of a method 4100 used for accelerated molecular discovery according to an example. As illustrated, method 4100 consists of three stages, where stage one relates to high-throughput virtual screening, stage two relates to artificial molecular design via a genetic algorithm and stage three relates to lead validation.

At 4102 (stage one), promising new core structure families that both allow for the design of INVEST emitters with appreciable OS and were likely realizable in a laboratory were identified. The bottom-up construction rules for molecules with inverted STGs was relied upon and 15 new core structure families predicted to have members being INVEST molecules were identified. In addition to the excited state properties of these molecules, the synthesizability and stability were assessed and Azulene was identified to be one of the most promising core structures. Since Azulenes are known to be very stable and are already widely used organic electronic materials, they were selected for further investigation.

Next, all 144 systematic permutations of core structure nitrogen substitutions of azulene were generated. The corresponding excited state properties of the generated permutations were simulated at the ωB2PLYP′, ADC(2), SOS-ADC(2) and EOM-CCSD levels of theory. The simulations revealed that only one of the nitrogen-substituted core structures, namely 2,5,7-triazaazulene was predicted to have an inverted STG at that level of theory. The 2,5,7-triazaazulene (molecule 1) structural family was accordingly selected for the next stage.

At 4104 (stage two), the artificial molecular design workflow was generated and implemented to find organic INVEST emitters. In the illustrated embodiments, the artificial molecular design workflow was generated using a genetic algorithm enhanced by neural networks and filters, as discussed in detail with reference to FIG. 42.

FIG. 42 illustrates a flowchart of a method 4200 of artificial molecular design according to an example. In the illustrated embodiment, an early development version of JANUS³⁸¹, an extension of a GA for inverse molecular design, that relies on the superfast traversal, optimization, novelty, exploration and discovery (STONED) algorithm for genetic operators and that only propagates one generation of molecules was used. An advantage of using this approach is that it can be applied to any molecular design problem with a well-defined fitness function without prior knowledge of the structural space to be investigated.

In the method 4200, the initial structural family to be investigated was received at 4202. Next, the fitness of the proposed molecules was investigated at 4204. In the illustrated embodiment, the fitness of the proposed molecules was evaluated based on simulating their excited state properties at the ωB2PLYP′ level of theory. In other embodiments, other fitness functions may be used. The fitness function is the function that is being optimized.

Based on the fitness investigation at 4204, the fit candidates were selected at 4206 and presented for molecular generation at 4208. In the illustrated embodiment, the self-referenced embedded strings for inference and evaluation of structures (SELFIES) and the STONED algorithm were used in the genetic operators. The STONED algorithm used string modifications in the SELFIES molecular representation of the fit candidates 4206 to generate suggestions 4210 for filters at 4212.

In the illustrated embodiment, filters 4212 were implemented and developed for cyclic π-systems as necessary requirements for every structure generated at 4210. This led to an increased sampling of the relevant structural space. Also in the illustrated embodiment, the filters 4212 were continuously updated to eliminate infeasible structures.

At 4214, if the molecular structure was found to be feasible, it was considered acceptable at 4216 and added to other acceptable molecules at 4218. The accepted molecules 4218 formed the next generation of the molecules to be investigated at 4202, and the method was repeated.

For a given set of parameters, such as, for example, fitness function and/or genetic operators 4220, method 4200 was repeated over multiple generations. In addition, method 4200 was repeated over multiple experiments, were the parameters (e.g. fitness function and/or genetic operators 4220) were varied over each experiment.

In the illustrated embodiment, in each run, the first 11 generations were proposed without the use of artificial neural networks (ANNs) enhancing sampling. Further, all molecules encountered until generation 11 were used to train ANN classifiers in each but the first experiment. This provided the benefit of identifying high-performing candidates at low computational cost and with high classification accuracy. The trained ANN classifiers were then incorporated into the genetic operators and used as additional classification filters 4212.

In the illustrated embodiment, only molecules identified as good were passed on to the fitness evaluation. This had the advantage of reducing the number of density functional theory (DFT) simulations for four subsequent generations, which tends to be costly. This additionally improved the exploration of promising candidates further.

In the illustrated embodiment, the size of the generated molecules was capped at 70 atoms, including hydrogens. This provided the advantage of avoiding prohibitively expensive quantum chemistry simulations.

Disclosed next is an example embodiment where method 4200 was repeated over multiple experiments. In this example embodiment, methane was used as a seed molecule for the first generation in the first artificial design experiment. Further, OS minus STG was used as fitness, with an upper STG threshold of 0.6 eV for high fitness. In this embodiment, apart from azulenes, several other known INVEST core structures were identified as promising candidates, including cyclobuta-1,3-diene, cycloocta-1,3,5,7-tetraene, pentalene, bowtiene, heptalene, zurlene and anthrazulene.³⁸² However, azulenes were selected for subsequent design efforts. In other embodiments, other core structures may be selected for subsequent design efforts.

Further in this example embodiment, molecule 1 was used as initial seed for the second, third and fourth artificial design experiments. In addition, only structures containing azulene-like π-systems were accepted in the molecular generation to ensure extensive exploration of that structural family. Further, the upper STG threshold for high fitness values was 0.3 eV in all these runs. In the second experiment, a linear combination of the additive inverse of the STG and OS was used as the fitness function. In the third experiment, only the OS was used as the fitness function. In the fourth experiment, the fitness was a linear combination of the additive inverse of the STG, the OS and the absolute difference to a VEE of 3.2 eV.

Next, to narrow down the space to be explored, focus on more promising structures and increase synthesizability, the molecules were constrained to possess an azulene-like π-systems and enforced to be identically substituted at the 1- and 6-positions in the fifth artificial design experiment. This was achieved by first generating the structures of the substituents which were subsequently attached to an azulene core structure only at the respective positions. Additionally, the linear combination of the additive inverse of the STG and OS was used as a fitness function in the fifth experiment.

Next, to increase the sampling of promising molecules even further, the structures with a plan of symmetry through the azulene core were enforced in the sixth artificial design experiment. Additionally, the core nitrogen substitutions equivalent to molecule 1 were kept in all proposed structures. Furthermore, only substitutions at the 4- and 8-positions were allowed as these would be preferred for the introduction of donor moieties based on the bottom-up design principles for INVEST emitters discussed above. This design space resulted in the best organic emitter candidates, with OSs reaching values far larger than 1. Additionally, even though the VEEs were not explicitly optimized in this run, a significant fraction of the structures had VEEs in the blue light region. Furthermore, the artificial design workflow disclosed here incorporated intramolecular hydrogen-bonding to the core nitrogen atoms in the most promising candidates. This has been proposed before as a very effective strategy to increase OSs of INVEST emitters.³⁸⁴

Referring back to FIG. 41, at 4106, promising INVEST emitter candidates (from all experiments) from step 4104 were validated. In the illustrated embodiment, the promising candidates were validated using quantum chemistry simulations at the SOS-ADC(2) level of theory. Molecules from all experiments were combined and Chimera³⁸³ was applied to scalarize multiple objectives and select the best-performing molecules based on the resulting rankings.

In the illustrated embodiment, two independent rankings were established. One ranking was based on both the STGs and Oss (objective A) and the other ranking was based on STGs, OSs and VEEs (objective B). In each of these rankings, the best 7,500 molecules were selected for further validation. Accounting for compounds that appeared in both rankings, a total set of 13,222 unique compounds were identified.

Based on the properties at the SOS-ADC(2) level, six of the best candidates for each of the two objectives were selected, as shown in Table 8. All these compounds emerged from experiment 6 and are likely stable. Additionally, the selected compounds all possess at least two hydrogen-bond donors allowing for intramolecular interactions controlling their conformations.

TABLE 8
Promising candidates after lead validation with their simulated properties at the SOS-
ADC(2)/cc-pVDZ level of theory. Objective A refers to the optimization of singlet-triplet gap and
oscillator strength, objective B refers to the optimization of singlet-triplet gap, oscillator strength
and vertical excitation energy.

| | | ΔE | | ΔE |
|---|---|---|---|---|
| | | (S₁-T₁) | | (S₀-S₁) |
| Objective | Molecule | [eV] | f₁₂ | [eV] |
| A | 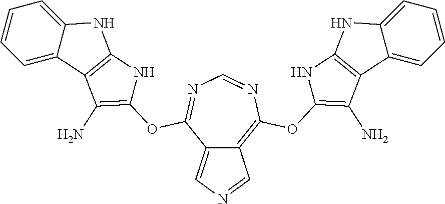 [SMILES: Nc1c(Oc2ncnc(Oc3[nH]c4[nH]c5ccccc5c4c3N)c3cncc2-3)[nH]c2[nH]c3ccccc3c12] | −0.01 | 0.401 | 2.26 |
| | 14 | | | |
| A | 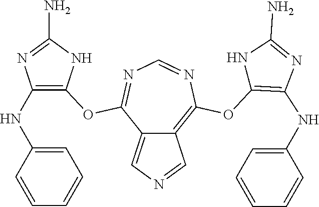 [SMILES: Nc1nc(Nc2ccccc2)c(Oc2ncnc(Oc3[nH]c(N)nc3Nc3ccccc3)c3cncc2-3)[nH]1] | −0.01 | 0.336 | 2.19 |
| | 15 | | | |
| A | 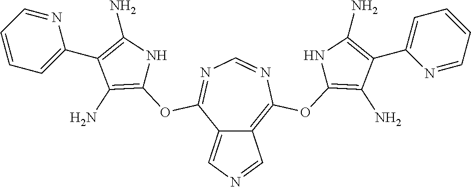 [SMILES: Nc1[nH]c(Oc2ncnc(Oc3[nH]c(N)c(-c4ccccn4)c3N)c3cncc2-3)c(N)c1-c1ccccn1] | −0.02 | 0.298 | 2.38 |
| | 16 | | | |
| A | 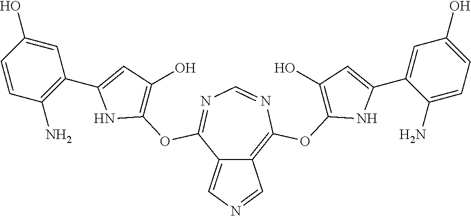 [SMILES: Nc1ccc(O)cc1-c1cc(O)c(Oc2ncnc(Oc3[nH]c(-c4cc(O)ccc4N)cc3O)c3cncc2-3)[nH]1] | −0.39 | 0.137 | 2.51 |
| | 17 | | | |
| A | 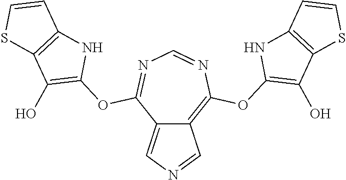 [SMILES: Oc1c(Oc2ncnc(Oc3[nH]c4ccsc4c3O)c3cncc2-3)[nH]c2ccsc12] | −0.11 | 0.169 | 2.50 |
| | 18 | | | |
| A | 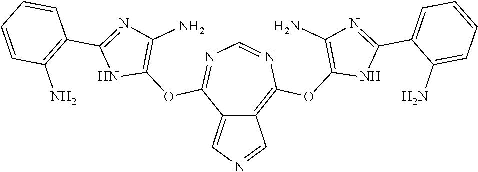 [SMILES: Nc1ccccc1-c1nc(N)c(Oc2ncnc(Oc3[nH]c(-c4ccccc4N)nc3N)c3cncc2-3)[nH]1] | −0.08 | 0.268 | 2.38 |
| | 19 | | | |
| B | 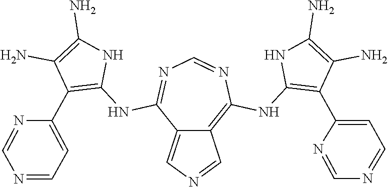 [SMILES: Nc1[nH]c(Nc2ncnc(Nc3[nH]c(N)c(N)c3-c3ccncn3)c3cncc2-3)c(-c2ccncn2)c1N] | −0.02 | 0.307 | 2.79 |
| | 20 | | | |
| B | 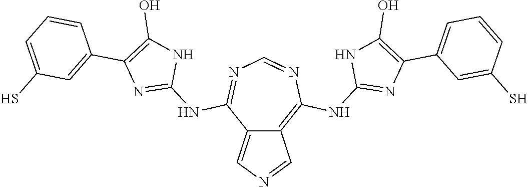 [SMILES: Oc1[nH]c(Nc2ncnc(Nc3nc(-c4cccc(S)c4)c(O)[nH]3)c3cncc2-3)nc1-c1cccc(S)c1] | −0.01 | 0.305 | 2.86 |
| | 21 | | | |
| B | 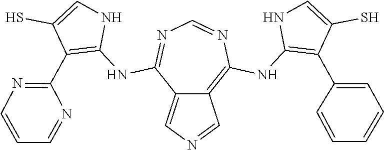 [SMILES: Sc1c[nH]c(Nc2ncnc(Nc3[nH]cc(S)c3-c3ncccn3)c3cncc2-3)c1-c1ccccc1] | −0.03 | 0.296 | 2.83 |
| | 22 | | | |
| B | 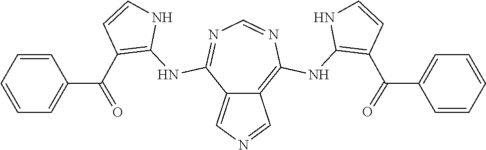 [SMILES: O=C(c1ccccc1)c1cc[nH]c1Nc1ncnc(Nc2[nH]ccc2C(=O)c2ccccc2)c2cncc1-2] | −0.11 | 0.121 | 2.79 |
| | 23 | | | |
| B | 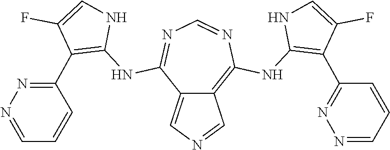 [SMILES: Fc1c[nH]c(Nc2ncnc(Nc3[nH]cc(F)c3-c3cccnn3)c3cncc2-3)c1-c1cccnn1] | −0.10 | 0.132 | 2.79 |
| | 24 | | | |
| B | 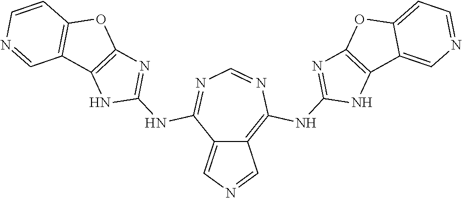 [SMILES: c1cc2oc3nc(Nc4ncnc(Nc5nc6oc7ccncc7c6[nH]5)c5cncc4-5)[nH]c3c2cn1] | −0.08 | 0.111 | 2.84 |
| | 25 | | | |

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATION OF DOCUMENTS CITED IN THE PRESENT APPLICATION

(1) Kutzelnigg, W.; Morgan, J. D. Z. Phys. D—At., Mol. Clusters 1996, 36, 197-214.
(2) Pauli, W. Z. Phys. 1925, 31, 765-783.
(3) Berson, J. A. In The Quinonoid Compounds (1988); John Wiley & Sons, Ltd, 1988; pp 455-536.
(4) Koseki, S. et al. Can. J. Chem. 1985, 63, 1572-1579.
(5) Toyota, A. Theoret. Chim. Acta 1988, 74, 209-217.
(6) Toyota, A.; Nakajima, T. J. Chem. Soc., Perkin Trans. 1986, 0, 1731-1734.
(7) Leupin, W.; Wirz, J. J. Am. Chem. Soc. 1980, 102, 6068-6075.
(8) Leupin, Werner. et al. J. Am. Chem. Soc. 1986, 108, 17-22.
(9) Naskar, S.; Das, M. ACS Omega 2017, 2, 1795-1803.
(10) Pios, S. et al. Physical Chemistry Chemical Physics 2021, 23, 12968-12975.
(11) Bonacic-Koutecky, V.; Michl, J. J. Am. Chem. Soc. 1985, 107, 1765-1766.
(12) Di, D. et al. Science 2017, 356, 159-163.
(13) de Silva, P. J. Phys. Chem. Lett. 2019, 10, 5674-5679.
(14) Ehrmaier, J. et al. J. Phys. Chem. A 2019, 123, 8099-8108.
(15) Ben Amor, N. et al. J. Chem. Phys. 2020, 153, 044118.
(16) Kollmar, H.; Staemmler, V. Theoret. Chim. Acta 1978, 48, 223-239.
(17) In Electron Spin Resonance Spectroscopy of Organic Radicals; John Wiley & Sons, Ltd, 2004; pp 49-82.
(18) de Silva, P. et al. Chem. Mater. 2019, 31, 6995-7006.
(19) Pollice, R. et al. Matter 2021, 4, 1654-1682.
(20) Sanz-Rodrigo, J. et al. J. Phys. Chem. A 2021, 125, 513-522.
(21) Ricci, G. et al. ChemPhysChem 2021, 22, 553-560.
(22) Sobolewski, A. L.; Domcke, W. J. Phys. Chem. Lett. 2021, 12, 6852-6860.
(23) Loken, C. et al. J. Phys.: Conf. Ser. 2010, 256, 012026.
(24) Ponce, M. et al. In Proceedings of the Practice and Experience in Advanced Research Computing on Rise of the Machines (learning); PEARC '19; Association for Computing Machinery: New York, NY, USA, 2019; pp 1-8.
(25) Pracht, P.; Grimme, S. Conformer-Rotamer Ensemble Sampling Tool. https://github.com/grimme-lab/crest (accessed Jun. 3, 2020).
(26) Grimme, S. J. Chem. Theory Comput. 2019, 15, 2847-2862.
(27) Pracht, P. et al. Phys. Chem. Chem. Phys. 2020, 22, 7169-7192.
(28) Grimme, S. et al. J. Chem. Theory Comput. 2017, 13, 1989-2009.
(29) Bannwarth, C. et al. J. Chem. Theory Comput. 2019, 15, 1652-1671.
(30) Spicher, S.; Grimme, S. Angew. Chem. Int. Ed. 2020, 59, 15665-15673.
(31) Spicher, S.; Grimme, S. J. Phys. Chem. Lett. 2020, 11, 6606-6611.
(32) Pracht, P. et al. J. Chem. Theory Comput. 2020, 16, 7044-7060.
(33) Grimme, S. Semiempirical Extended Tight-Binding Program Package https://github.com/grimme-lab/xtb (accessed Jun. 3, 2020).
(34) Neese, F. WIREs Comput. Mol. Sci. 2018, 8, e1327.
(35) Neese, F. WIREs Comput. Mol. Sci. 2012, 2, 73-78.
(36) Brandenburg, J. G. et al. J. Chem. Phys. 2018, 148, 064104.
(37) Casanova-Paez, M. et al. J. Chem. Theory Comput. 2019, 15, 4735-4744.
(38) Weigend, F.; Ahlrichs, R. Phys. Chem. Chem. Phys. 2005, 7, 3297-3305.
(39) Krylov, A. I. Chem. Phys. Lett. 2001, 350, 522-530.
(40) Zhang, X.; Herbert, J. M. J. Chem. Phys. 2015, 143, 234107.
(41) Perdew, J. P. et al. Phys. Rev. Lett. 1996, 77, 3865-3868.
(42) Perdew, J. P. et al. J. Chem. Phys. 1996, 105, 9982-9985.
(43) Adamo, C.; Barone, V. J. Chem. Phys. 1999, 110, 6158-6170.
(44) Bernard, Y. A. et al. J. Chem. Phys. 2012, 136, 204103.
(45) Dunning, T. H. J. Chem. Phys. 1989, 90, 1007-1023.
(46) Becke, A. D. Phys. Rev. A 1988, 38, 3098-3100.
(47) Lee, C. et al. Phys. Rev. B 1988, 37, 785-789.
(48) Becke, A. D. J. Chem. Phys. 1993, 98, 5648-5652.
(49) Ditchfield, R. et al. J. Chem. Phys. 1971, 54, 724-728.
(50) Hehre, W. J. et al. J. Chem. Phys. 1972, 56, 2257-2261.
(51) Hariharan, P. C.; Pople, J. A. Theoret. Chim. Acta 1973, 28, 213-222.
(52) Shao, Y. et al. Mol. Phys. 2015, 113, 184-215.
(53) Nielsen, E. S. et al. J. Chem. Phys. 1980, 73, 6238-6246.
(54) Sauer, S. P. A. J. Phys. B: At. Mol. Opt. Phys. 1997, 30, 3773-3780.
(55) Eriksen, J. J. et al. J. Comput. Chem. 2012, 33, 2012-2022.
(56) Schirmer, J. Phys. Rev. A 1982, 26, 2395-2416.
(57) Trofimov, A. B.; Schirmer, J. J. Phys. B: At. Mol. Opt. Phys. 1995, 28, 2299-2324.
(58) Starcke, J. H. et al. J. Chem. Phys. 2009, 130, 024104.
(59) Wormit, M. et al. Mol. Phys. 2014, 112, 774-784.
(60) Hattig, C.; Weigend, F. J. Chem. Phys. 2000, 113, 5154-5161.
(61) Christiansen, O. et al. Chem. Phys. Lett. 1995, 243, 409-418.
(62) Mester, D.; Kállay, M. J. Chem. Theory Comput. 2019, 15, 4440-4453.
(63) Mester, D. et al. J. Chem. Phys. 2017, 146, 194102.
(64) Mester, D. et al. J. Chem. Phys. 2018, 148, 094111.
(65) Mester, D.; Kállay, M. J. Chem. Theory Comput. 2019, 15, 1690-1704.
(66) Mester, D. et al. J. Chem. Theory Comput. 2019, 15, 6111-6126.
(67) Winter, N. O. C.; Hattig, C. J. Chem. Phys. 2011, 134, 184101.
(68) Grimme, S. J. Chem. Phys. 2003, 118, 9095-9102.
(69) Hellweg, A. et al. Phys. Chem. Chem. Phys. 2008, 10, 4119-4127.
(70) Head-Gordon, M. et al. Chem. Phys. Lett. 1994, 219, 21-29.
(71) Head-Gordon, M. et al. Chem. Phys. Lett. 1995, 246, 114-121.
(72) Rhee, Y. M.; Head-Gordon, M. J. Phys. Chem. A 2007, 111, 5314-5326.
(73) Kállay, M. et al. MRCC, a quantum chemical program suite, www.mrcc.hu.
(74) Kállay, M. et al. J. Chem. Phys. 2020, 152, 074107.
(75) Grimme, S.; Hansen, A. Angew. Chem. Int. Ed. 2015, 54, 12308-12313.
(76) Casanova-Páez, M.; Goerigk, L. J. Chem. Phys. 2020, 153, 064106.
(77) Convert Cartesian coordinates to one or more https://github.com/jensengroup/xyz2 mol (accessed May 3, 2022).
(78) RDKit https://github.com/rdkit/rdkit (accessed May 3, 2022).
(79) Borden, W. T. et al. Acc. Chem. Res. 1994, 27, 109-116.
molecular graphs
(80) Hrovat, D. A.; Borden, W. T. J. Mol. Struct. THEOCHEM 1997, 398-399, 211-220.
(81) Zilberg, S.; Haas, Y. J. Phys. Chem. A 1998, 102, 10851-10859.
(82) Borden, W. T. J. Am. Chem. Soc. 1975, 97, 5968-5970.
(83) Ni [symbol] ă, M. et al. Phys. Rev. B 2017, 96, 235101.
(84) Bally, T.; Borden, W. T. In Reviews in Computational Chemistry; John Wiley & Sons, Ltd, 2007; pp 1-97.
(85) Gómez-Bombarelli, R. et al. Nat. Mater. 2016, 15, 1120-1127.
(86) Zahradník, R. Angew. Chem. Int. Ed. Engl. 1965, 4, 1039-1050.
(87) Lewars, E. Can. J. Chem. 2014, 92, 378-385.
(88) Duc, L. et al. Tetrahedron Lett. 1968, 9, 6173-6178.
(89) Hertwig, R. H. et al. Angew. Chem. Int. Ed. Engl. 1994, 33, 1192-1194.
(90) Bell, W. L. et al. Organometallics 1987, 6, 266-273.
(91) Bertelli, D. J. J. Org. Chem. 1964, 29, 3032-3036.
(92) Prinzbach, H.; Schneider, H.-W. Angew. Chem. Int. Ed. Engl. 1973, 12, 1007-1009.
(93) Dewar, M. J. S.; Trinajstić, N. Tetrahedron Lett. 1969, 10, 2129-2132.
(94) Oda, M. et al. Angew. Chem. Int. Ed. 2001, 40, 2660-2662.
(95) Thomas, P. M.; Weber, A. J. Raman Spectrosc. 1978, 7, 353-357.
(96) Claus, K. H.; Krüger, C. Acta Cryst C 1988, 44, 1632-1634.
(97) Lindner, H. J.; Kitschke, B. Angew. Chem. 1976, 88, 123-124.
(98) Stegemann, J.; Lindner, H. J. Tetrahedron Lett. 1977, 18, 2515-2516.
Li, J. et al. Dyes Pigm. 2022, 203, 110366. (99)
(100) Borden, W. T.; Davidson, E. R. J. Am. Chem. Soc. 1977, 99, 4587-4594.
(101) Carissan, Y. et al. J. Phys. Chem. A 2008, 112, 13256-13262.
(102) Carissan, Y. et al. L'Actualité Chimique 2016, 406, 36-40.
(103) Humbel, A. contribution from C., Aix Marseille University, Department of Chemistry, Nicolas Goudard, Yannick Carissan, Denis Hagebaum-Reignier, Stéphane. Huckel Theory and HuLiS: a calculator that also describes mesomerism. http://www.hulis.free.fr/(accessed Jun. 9, 2022).
(104) Gompper, R.; Wagner, H.-U. Angew. Chem. Int. Ed. Engl. 1988, 27, 1437-1455.
(105) Manatt, S.; Roberts, J. J. Org. Chem. 1959, 24, 1336-1338.
(106) Hoffmann, R. J. Chem. Soc. D 1969, No. 5, 240-241.
(107) Hess, B. A.; Schaad, L. J. J. Org. Chem. 1976, 41, 3058-3059.
(108) Weiss, R.; Murrell, J. N. Tetrahedron 1971, 27, 2877-2883.
(109) Hafner, K. et al. Angew. Chem. Int. Ed. Engl. 1963, 2, 123-134.
(110) Hafner, K. Angew. Chem. Int. Ed. Engl. 1964, 3, 165-173.
(111) Gimarc, B. M. J. Am. Chem. Soc. 1983, 105, 1979-1984.
(112) Sancho-García, J. C. et al. J. Chem. Phys. 2022, 156, 034105.
(113) Alipour, M.; Izadkhast, T. J. Chem. Phys. 2022, 156, 064302.
(114) Omist, A. et al. Phys. Chem. Chem. Phys. 2021, 23, 24016-24028.
(115) Ghosh, S.; Bhattacharyya, K. J. Phys. Chem. A 2022, 126, 1378-1385.
(116) Sancho-García, J.-C.; San-Fabián, E. Computation 2022, 10, 13.
(117) Dinkelbach, F. et al. J. Phys. Chem. A 2021, 125, 10044-10051.
(118) ROWE, D. J. Rev. Mod. Phys. 1968, 40, 153-166.
(119) Emrich, K. Nucl. Phys. A 1981, 351, 379-396.
(120) Geertsen, J. et al. Chem. Phys. Lett. 1989, 164, 57-62.
(121) Stanton, J. F.; Bartlett, R. J. J. Chem. Phys. 1993, 98, 7029-7039.
(122) Krylov, A. I. Annu. Rev. Phys. Chem. 2008, 59, 433-462.
(123) Landau, A. et al. J. Chem. Phys. 2010, 132, 014109.
(124) Sosa, C. et al. Chem. Phys. Lett. 1989, 159, 148-154.
(125) Taube, A. G.; Bartlett, R. J. Collect. Czech. Chem. Commun. 2005, 70, 837-850.
(126) Taube, A. G.; Bartlett, R. J. J. Chem. Phys. 2008, 128, 164101.
(127) Maier, G. Angew. Chem. Int. Ed. Engl. 1974, 13, 425-438.
(128) Bally, T.; Masamune, S. Tetrahedron 1980, 36, 343-370.
(129) Maier, G. Angew. Chem. Int. Ed. Engl. 1988, 27, 309-332.
(130) Johnson, R. P. In The Chemistry of Cyclobutanes; John Wiley & Sons, Ltd, 2005; pp 589-616.
(131) Watts, L. et al. J. Am. Chem. Soc. 1965, 87, 3253-3254.
(132) Hedaya, E. et al. J. Am. Chem. Soc. 1969, 91, 1875-1877.
(133) Avram, M. et al. Chem. Ber. 1964, 97, 382-389.
(134) Maier, G.; Hoppe, B. Tetrahedron Lett. 1973, 14, 861-864.
(135) Chapman, O. L. et al. J. Am. Chem. Soc. 1973, 95, 614-617.
(136) Lin, C. Y.; Krantz, A. J. Chem. Soc., Chem. Commun. 1972, No. 19, 1111-1112.
(137) Cram, D. J. et al. Angew. Chem. Int. Ed. Engl. 1991, 30, 1024-1027.
(138) Skell, P. S.; Petersen, R. J. J. Am. Chem. Soc. 1964, 86, 2530-2531.
(139) Maier, G.; Alzérreca, A. Angew. Chem. Int. Ed. Engl. 1973, 12, 1015-1016.
(140) Maier, G. et al. Angew. Chem. Int. Ed. Engl. 1978, 17, 520-521.
(141) Maier, G. et al. Angew. Chem. 1970, 82, 551-552.
(142) Eisenbarth, P.; Regitz, M. Chem. Ber. 1982, 115, 3796-3810.
(143) Irngartinger, H. et al. Angew. Chem. Int. Ed. Engl. 1980, 19, 211-212.
(144) Irngartinger, H.; Rodewald, H. Angew. Chem. Int. Ed. Engl. 1974, 13, 740-741.
(145) Delbaere, L. T. J. et al. J. Am. Chem. Soc. 1975, 97, 1973-1974.
(146) Borden, W. T.; Davidson, E. R. J. Am. Chem. Soc. 1980, 102, 7958-7960.
(147) Breslow, R. et al. J. Am. Chem. Soc. 1965, 87, 5132-5139.
(148) Politzer, P. et al. Can. J. Chem. 1993, 71, 1123-1127.
(149) Gompper, R.; Seybold, G. Angew. Chem. 1968, 80, 804-806.
(150) Neuenschwander, M.; Niederhauser, A. Helv. Chim. Acta 1970, 53, 519-531.
(151) Lindner, H. J.; von Gross, B. Angew. Chem. 1971, 83, 489-490.
(152) Gompper, R. et al. Angewandte Chemie 1975, 87, 711-712.
(153) Gompper, R. et al. Tetrahedron 1976, 32, 629-638.
(154) Dewar, M. J. S.; Trinajstić, N. Theoret. Chim. Acta 1970, 17, 235-238.
(155) Vogelbacher, U.-J. et al. Angew. Chem. 1986, 98, 835-836.
(156) Seybold, G. et al. Angew. Chem. 1973, 85, 918-920.
(157) Wagner, H.-U. Angew. Chem. 1973, 85, 920-921.
(158) Chambers, R. D. et al. J. Chem. Soc., Chem. Commun. 1987, No. 22, 1699-1700.
(159) Chambers, R. D.; Maslakiewicz, J. R. J. Chem. Soc., Chem. Commun. 1976, No. 24,
1005-1006.
(160) Shoji, Y. et al. Angew. Chem. Int. Ed. 2021, 60, 21817-21823.
(161) Wong, M. Y.; Zysman-Colman, E. Adv. Mater. 2017, 29, 1605444.
(162) Liu, Y. et al. Nat Rev. Mater. 2018, 3, 1-20.
(163) Konidena, R. K.; Lee, J. Y. Chem. Rec. 2019, 19, 1499-1517.
(164) Kaufman, H. S. et al. Nature 1948, 161, 165-165.
(165) Prosen, E. J. et al. J. Am. Chem. Soc. 1947, 69, 2068-2069.
(166) Willstätter, R.; Waser, E. Ber. Dtsch. Chem. Ges. 1911, 44, 3423-3445.
(167) Stinson, M. et al. Plant Sci. 2003, 165, 913-922.
(168) Paquette, L. A. Tetrahedron 1975, 31, 2855-2883.
(169) Wang, C.; Xi, Z. Chem. Commun. 2007, No. 48, 5119-5133.
(170) Nishinaga, T. Sci. Synth. 2010, 45, 383.
(171) Paquette, L. A.; Kakihana, T. J. Am. Chem. Soc. 1968, 90, 3897-3898.
(172) Paquette, L. A. et al. J. Am. Chem. Soc. 1971, 93, 152-161.
(173) Al-Jalal, N. et al. J. Chem. Soc., Chem. Commun. 1985, No. 2, 85-86.
(174) Al-Jalal, N.; Gilbert, A. Recueil des Travaux Chimiques des Pays-Bas 1990, 109, 21-25.
(175) Šket, B.; Zupan, M. Tetrahedron 1989, 45, 1755-1758.
(176) Al-Jalal, N. et al. Tetrahedron 1988, 44, 1449-1459.
(177) Maier, G. et al. Chem. Ber. 1981, 114, 3922-3934.
(178) Hedaya, E. et al. J. Am. Chem. Soc. 1971, 93, 3817-3818.
(179) Elix, J. A. et al. Aust. J. Chem. 1972, 25, 865-874.
(180) Elix, J. A. et al. Tetrahedron Lett. 1970, 11, 1837-1840.
(181) Turro, N. J. et al. J. Am. Chem. Soc. 1976, 98, 4320-4322.
(182) Trost, B. M.; Cory, R. M. J. Am. Chem. Soc. 1971, 93, 5573-5575.
(183) Yogi, S. et al. Bull. Chem. Soc. Jpn. 1986, 59, 1087-1094.
(184) Yogi, S. et al. Bull. Chem. Soc. Jpn. 1987, 60, 731-735.
(185) Yogi, S. et al. Bull. Chem. Soc. Jpn. 1987, 60, 335-342.
(186) Burgess, E. M.; Sanchez, J. P. J. Org. Chem. 1974, 39, 940-948.
(187) Yogi, S. et al. Bull. Chem. Soc. Jpn. 1987, 60, 343-347.
(188) Burgess, E. M.; Sanchez, J. P. J. Org. Chem. 1973, 38, 176-178.
(189) Maier, G. et al. Liebigs Ann. 1995, 1995, 173-186.
(190) Zhang, S. et al. J. Am. Chem. Soc. 2012, 134, 11964-11967.
(191) Düll, B.; Müllen, K. Tetrahedron Lett. 1992, 33, 8047-8050.
(192) Trost, B. M. et al. J. Org. Chem. 1979, 44, 1264-1269.
(193) Schnieders, C. et al. Angew. Chem. Int. Ed. Engl. 1985, 24, 576-577.
(194) Beyer, H.; Pyl, T. Justus Liebigs Ann. Chem. 1957, 605, 50-58.
(195) Furukawa, M. et al. Chem. Pharm. Bull. 1972, 20, 927-930.
(196) Beyer, H.; Pyl, Th. Angew. Chem. 1956, 68, 374-375.
(197) Pfleger, R. et al. Angew. Chem. 1956, 68, 680-681.
(198) Pfleger, R.; Hahn, H.-G. Chem. Ber. 1957, 90, 2411-2416.
(199) Pfleger, R. et al. Chem. Ber. 1963, 96, 1827-1839.
(200) Eisch, J. J. et al. Eur. J. Org. Chem. 2008, 2008, 392-397.
(201) Gompper, R. et al. Angew. Chem. 1987, 99, 1071-1073.
(202) Ehrenberg, S. et al. Angew. Chem. Int. Ed. Engl. 1991, 30, 334-336.
(203) Gompper, R.; Schwarzensteiner, M.-L. Angew. Chem. 1983, 95, 553-554.
(204) Mai, V. T. N. et al. Nat. Commun. 2020, 11, 5623.
(205) Pappalardo, R. et al. Appl. Phys. Lett. 1970, 16, 267-269.
(206) Marling, J. B. et al. Appl. Phys. Lett. 1970, 17, 527-530.
(207) Dempster, D. N. et al. Journal of Photochemistry 1973, 2, 343-359.
(208) Yamashita, M.; Kashiwagi, H. IEEE Journal of Quantum Electronics 1976, 12, 90-95.
(209) Zietek, B. et al. In Laser Technology II; SPIE, 1987; Vol. 0859, pp 25-29.
(210) Lu, P. et al. J. Am. Chem. Soc. 2000, 122, 7480-7486.
(211) Palmer, M. H. et al. J. Chem. Phys. 2019, 151, 084304.
(212) Herndon, W. C.; Ellzey, M. L. J. Am. Chem. Soc. 1974, 96, 6631-6642.
(213) Randic, M. J. Am. Chem. Soc. 1977, 99, 444-450.
(214) Hafner, K. et al. Angew. Chem. Int. Ed. Engl. 1973, 12, 337-339.
(215) De Mayo, P. et al. J. Am. Chem. Soc. 1971, 93, 3071-3072.
(216) Bally, T. et al. J. Am. Chem. Soc. 1997, 119, 1869-1875.
(217) Le Goff, Eugene. J. Am. Chem. Soc. 1962, 84, 3975-3976.
(218) Hafner, K.; Süss, H. U. Angew. Chem. Int. Ed. Engl. 1973, 12, 575-577.
(219) Kitschke, B.; Lindner, H. J. Tetrahedron Lett. 1977, 18, 2511-2514.
(220) Hafner, K. et al. Angew. Chem. Int. Ed. Engl. 1967, 6, 451-452.
(221) Hartke, K.; Marusch, R. Chem. Ber. 1972, 105, 2584-2593.
(222) Hafner, K.; Schmidt, F. Angew. Chem. Int. Ed. Engl. 1973, 12, 418-419.
(223) Gais, H.-J.; Hafner, K. Tetrahedron Lett. 1974, 15, 771-774.
(224) Hafner, K. Pure Appl. Chem. 1982, 54, 939-956.
(225) Closs, F.; Gompper, R. Angew. Chem. Int. Ed. Engl. 1987, 26, 552-554.
(226) Closs, F. et al. Angew. Chem. Int. Ed. Engl. 1988, 27, 842-845.
(227) Karaghiosoff, K. et al. Chem. Ber. 1986, 119, 3213-3226.
(228) Kasha, M. Discuss. Faraday Soc. 1950, 9, 14-19.
(229) Bearpark, M. J. et al. Int. J. Quantum Chem. 1996, 60, 505-512.
(230) Falchi, A. et al. J. Phys. Chem. A 1998, 102, 5006-5012.
(231) Bearpark, M. J.; Robb, M. A. J. Phys. Chem. A 2000, 104, 1075-1077.
(232) Falchi, A. et al. J. Phys. Chem. A 2000, 104, 1078-1078.
(233) Nölle, D.; Nöth, H. Z. Naturforsch. B 1972, 27, 1425-1427.
(234) Groh, T. et al. Organometallics 2000, 19, 2477-2481.
(235) Kashida, J. et al. Angew. Chem. Int. Ed. 2021, 60, 23812-23818.
(236) Hess, B. A.; Schaad, L. J. J. Org. Chem. 1971, 36, 3418-3423.
(237) Zhou, Z.; Parr, R. G. J. Am. Chem. Soc. 1989, 111, 7371-7379.
(238) Gordon, Maxwell. Chem. Rev. 1952, 50, 127-200.
(239) St. Pfau, A.; Plattner, PI. A. Helv. Chim. Acta 1939, 22, 202-208.
(240) Abou-Hadeed, K.; Hansen, H.-J. Sci. Synth. 2010, 45, 1043.
(241) Shoji, T. et al. International Journal of Molecular Sciences 2021, 22, 10686.
(242) Elwahy, A. H. M. et al. ChemistrySelect 2021, 6, 13664-13723.
(243) Shi, X. et al. Chemistry—An Asian Journal 2018, 13, 143-157.
(244) Shoji, T. et al. International Journal of Molecular Sciences 2020, 21, 7087.
(245) Elwahy, A. H. M.; Hafner, K. Asian Journal of Organic Chemistry 2021, 10, 2010-2083.
(246) Mochalin, V. B.; Porshnev, Y. N. Russ. Chem. Rev. 1977, 46, 530.
(247) Razus, A. C. Symmetry 2022, 14, 297.
(248) Liu, R. S. H.; Asato, A. E. Journal of Photochemistry and Photobiology C: Photochemistry Reviews 2003, 4, 179-194.
(249) Ou, L. et al. Chinese Chemical Letters 2019, 30, 1903-1907.
(250) Ito, S. et al. Synlett 2011, 2011, 2279-2298.
(251) Ito, S.; Morita, N. European Journal of Organic Chemistry 2009, 2009, 4567-4579.
(252) Murfin, L. C.; Lewis, S. E. Molecules 2021, 26, 353.
(253) Xin, H. et al. Acc. Chem. Res. 2021, 54, 1737-1753.
(254) Dong, J.-X.; Zhang, H.-L. Chin. Chem. Lett. 2016, 27, 1097-1104.
(255) Huang, J. et al. Small Methods 2020, 4, 2000628.
(256) Shoji, T.; Ito, S. Chem. Eur. J. 2017, 23, 16696-16709.
(257) Zeng, H. N. et al. Chemistry-An Asian Journal 2020, 15, 1904-1915.
(258) Lvov, A. G.; Bredihhin, A. Org. Biomol. Chem. 2021, 19, 4460-4468.
(259) Xin, H.; Gao, X. ChemPlusChem 2017, 82, 945-956.
(260) Beer, M.; Longuet-Higgins, H. C. J. Chem. Phys. 1955, 23, 1390-1391.
(261) Viswanath, G.; Kasha, M. J. Chem. Phys. 1956, 24, 574-577.
(262) Bearpark, M. J. et al. J. Am. Chem. Soc. 1996, 118, 169-175.
(263) Prlj, A. et al. J. Chem. Theory Comput. 2020, 16, 2617-2626.
(264) Amatatsu, Y.; Komura, Y. J. Chem. Phys. 2006, 125, 174311.
(265) Diau, E. W.-G. et al. J. Chem. Phys. 1999, 110, 9785-9788.
(266) Kröning, P.; Glandien, M. Zeitschrift für Physikalische Chemie 1970, 72, 76-81.
(267) Eber, G. et al. Chemical Physics Letters 1974, 29, 397-404.
(268) Eber, G. et al. Journal of Photochemistry 1977, 7, 91-99.
(269) Griesser, H. J.; Wild, U. P. Journal of Photochemistry 1980, 13, 309-318.
(270) Griesser, H. J.; Thistlethwaite, P. J. Journal of Photochemistry and Photobiology A: Chemistry 1988, 43, 247-261.
(271) Burke, F. P. et al. Chemical Physics Letters 1973, 19, 574-579.
(272) Braun, J. R. et al. J. Chem. Phys. 1973, 59, 3595-3599.
(273) Small, G. J.; Burke, F. P. J. Chem. Phys. 1977, 66, 1767-1778.
(274) Jinguji, M. et al. Chemical Physics Letters 1985, 121, 400-404.
(275) Vosskötter, S. et al. Phys. Chem. Chem. Phys. 2015, 17, 23573-23581.
(276) Glandien, M.; Kröning, P. 1970, 71, 149-152.
(277) Herkstroeter, W. G. J. Am. Chem. Soc. 1975, 97, 4161-4167.
(278) Gorman, A. A. et al. J. Am. Chem. Soc. 1984, 106, 6952-6955.
(279) Nickel, B. Zeitschrift für Physikalische Chemie 1992, 178, 25-40.
(280) Huzak, M. et al. Chemical Physics 2012, 406, 55-64.
(281) Surman, M. D.; Hutchings, R. H. Sci. Synth. 2004, 17, 749-823.
(282) Abe, N.; Gunji, T. HETEROCYCLES 2010, 82, 201.
(283) Myers, A. G. et al. J. Org. Chem. 2004, 69, 2516-2525.
(284) Eaton, P. E. J. Am. Chem. Soc. 1962, 84, 2344-2348.
(285) Bister, H.-J.; Butenschön, H. Synlett 1992, 1992, 22-24.
(286) Aihara, J. Phys. Chem. Chem. Phys. 2016, 18, 11847-11857.
(287) Gastmans, J. P. et al. Tetrahedron 1977, 33, 2205-2213.
(288) Gastmans, J. P. et al. Can. J. Chem. 1979, 57, 2864-2868.
(289) Nakajima, T. Pure Appl. Chem. 1971, 28, 219-238.
(290) Vogel, E. et al. Angew. Chem. 1974, 86, 777-778.
(291) Briquet, A. A. S. et al. Helv. Chim. Acta 1996, 79, 2282-2315.
(292) Gottarelli, G. et al. Helv. Chim. Acta 1987, 70, 430-435.
(293) Bernhard, W. et al. Helv. Chim. Acta 1985, 68, 415-428.
(294) Hafner, K. et al. Angew. Chem. 1985, 97, 209-213.
(295) Hafner, K. et al. Bull. Chem. Soc. Jpn. 1988, 61, 155-163.
(296) Dauben, H. J.; Bertelli, D. J. J. Am. Chem. Soc. 1961, 83, 4659-4660.
(297) Boehm, M. C.; Schuett, J. J. Phys. Chem. 1992, 96, 3674-3683.
(298) Vogel, E.; Ippen, J. Angew. Chem. 1974, 86, 778-779.
(299) Vogel, E.; Hogrefe, F. Angew. Chem. 1974, 86, 779-780.
(300) Ruske, W. J. Prakt. Chem. 1962, 18, 173-174.
(301) Kataoka, M. et al. J. Org. Chem. 1986, 51, 358-362.
(302) Kataoka, M.; Nakajima, T. Bull. Chem. Soc. Jpn. 1992, 65, 2371-2374.
(303) Ott, P.; Hansen, H.-J. Helv. Chim. Acta 2001, 84, 2670-2692.
(304) Landmesser, T. et al. Helv. Chim. Acta 2013, 96, 1851-1893.
(305) Abou-Hadeed, K. et al. Helv. Chim. Acta 2012, 95, 885-921.
(306) Rippert, A. J. et al. Helv. Chim. Acta 1993, 76, 2876-2886.
(307) Klärner, F.-G. et al. Angew. Chem. Int. Ed. Engl. 1982, 21, 708-709.
(308) Hafner, K. et al. Angew. Chem. 1976, 88, 121-123.
(309) Paquette, L. A. et al. Angew. Chem. 1979, 91, 581-582.
(310) Weber, R. H. et al. Helv. Chim. Acta 1987, 70, 1439-1460.
(311) Uebelhart, P. et al. Helv. Chim. Acta 1995, 78, 1437-1464.
(312) Briling, K. R.; Laikov, D. N. Russ. J. Org. Chem. 2020, 56, 569-575.
(313) Bernhard, W. et al. Helv. Chim. Acta 1985, 68, 429-438.
(314) Weber, R. H. et al. Helv. Chim. Acta 1987, 70, 742-759.
(315) Houar, S. E.; Hansen, H.-J. Helv. Chim. Acta 1997, 80, 253-266.
(316) Song, J.; Hansen, H.-J. Helv. Chim. Acta 1999, 82, 2260-2273.
(317) Rippert, A. J.; Hansen, H.-J. Helv. Chim. Acta 1992, 75, 2219-2226.
(318) Lellek, V.; Hansen, H.-J. Helv. Chim. Acta 2001, 84, 1712-1736.
(319) Maillefer-El Houar, S. et al. Helv. Chim. Acta 2013, 96, 1488-1541.
(320) Hafner, K.; Knaup, G. L. Tetrahedron Lett. 1986, 27, 1665-1668.
(321) Jin, X. et al. Helv. Chim. Acta 2010, 93, 729-745.
(322) Song, J.; Hansen, H.-J. Helv. Chim. Acta 1999, 82, 1690-1706.
(323) Guspanová, J. et al. Helv. Chim. Acta 1997, 80, 1375-1407.
(324) Ochertyanova, E. A.; Hansen, H.-J. Helv. Chim. Acta 2002, 85, 1128-1139.
(325) Hafner, K. et al. Tetrahedron Lett. 1986, 27, 1669-1672.
(326) Chen, Y.; Hansen, H.-J. Helv. Chim. Acta 1993, 76, 168-177.
(327) Chen, Y. et al. Helv. Chim. Acta 1992, 75, 2447-2492.
(328) Uebelhart, P.; Hansen, H.-J. Helv. Chim. Acta 1992, 75, 2493-2514.
(329) Hafner, K.; Knaup, G. L. Tetrahedron Lett. 1986, 27, 1673-1676.
(330) Bernhard, W. et al. Helv. Chim. Acta 1985, 68, 1010-1024.
(331) Zahradník, R. et al. Collect. Czech. Chem. Commun. 1966, 31, 640-648.
(332) Soriano Jartín, R. et al. J. Phys. Chem. A 2002, 106, 11806-11814.
(333) Nendel, M. et al. J. Mol. Struct. 1999, 461-462, 23-28.
(334) Hafner, K.; Krimmer, H.-P. Angew. Chem. 1980, 92, 202-204.
(335) Hafner, K. et al. Angew. Chem. Int. Ed. Engl. 1986, 25, 630-632.
(336) Klann, R. et al. Chem. Phys. Lett. 1990, 169, 172-178.
(337) Gellini, C. et al. J. Phys. Chem. 1993, 97, 8152-8157.
(338) Gellini, C. et al. J. Phys. Chem. 1995, 99, 85-93.
(339) Bearpark, M. J. et al. Mol. Phys. 1999, 96, 645-652.
(340) Closs, F. et al. Angew. Chem. Int. Ed. Engl. 1987, 26, 1037-1039.
(341) Schardt, S. et al. Angew. Chem. Int. Ed. 1995, 34, 330-332.
(342) Schardt, S.; Hafner, K. Tetrahedron Lett. 1996, 37, 3829-3832.
(343) Hanida, K. et al. Angew. Chem. Int. Ed. 2021, 60, 20765-20770.
(344) Aihara, J. J. Am. Chem. Soc. 1976, 98, 2750-2758.
(345) Wilcox, C. F. J. Am. Chem. Soc. 1969, 91, 2732-2736.
(346) Prinzbach, H. Pure Appl. Chem. 1971, 28, 281-330.
(347) Sauter, H. et al. Chem. Ber. 1977, 110, 1382-1402.
(348) Prinzbach, H.; Knothe, L. Pure Appl. Chem. 1986, 58, 25-37.
(349) Wiersum, U. E.; Jenneskens, L. W. Tetrahedron Lett. 1993, 34, 6615-6618.
(350) Shukla, B. et al. J. Phys. Chem. A 2011, 115, 5284-5293.
(351) Campbell, A. D.; Slater, S. N. J. Chem. Soc. 1952, No. 0, 4353-4357.
(352) Bertelli, D. J. J. Org. Chem. 1965, 30, 891-895.
(353) Bordwell, F. G.; Winn, M. J. Org. Chem. 1967, 32, 42-49.
(354) Patwardhan, S. A.; Dev, S. Tetrahedron 1972, 28, 1075-1082.
(355) Maitlis, P. M. In Proceedings of the Chemical Society, The Royal Society of Chemistry, 1957; pp 329-368.
(356) Prinzbach, H. et al. Tetrahedron Lett. 1978, 19, 649-652.
(357) Bingmann, H. et al. Chem. Ber. 1981, 114, 1679-1696.
(358) Prinzbach, H. et al. Chem. Ber. 1981, 114, 1697-1722.
(359) Glidewell, C.; Lloyd, D. Tetrahedron 1984, 40, 4455-4472.
(360) Bird, C. W. Tetrahedron 1998, 54, 10179-10186.
(361) Hafner, K. et al. Angew. Chem. Int. Ed. Engl. 1988, 27, 1191-1192.
(362) Pariser, R.; Parr, R. G. J. Chem. Phys. 1953, 21, 466-471.
(363) Pariser, R.; Parr, R. G. J. Chem. Phys. 1953, 21, 767-776.
(364) Pople, J. A. Trans. Faraday Soc. 1953, 49, 1375-1385.
(365) Schweikert, O. et al. Chem. Ber. 1984, 117, 2006-2026
(366) Charaf-Eddin, A. et al. J. Chem. Theory Comput. 2013, 9, 2749-2760.
(367) Humeniuk, A. et al. J. Chem. Phys. 2020, 152, 054107.
(368) Theil, H. Indag. Math. 1950, 12, 173.
(369) Sen, P. K. J. Am. Stat. Assoc. 1968, 63, 1379-1389.
(370) Roos, B. O. In Advances in Chemical Physics; John Wiley & Sons, Ltd, 1987; pp 399-445.
(371) Roos, B. O. et al. Chem. Phys. 1982, 66, 197-207.
(372) Andersson, Kerstin. et al. J. Phys. Chem. 1990, 94, 5483-5488.
(373) Andersson, K. Theoret. Chim. Acta 1995, 91, 31-46.
(374) Angeli, C. et al. J. Chem. Phys. 2001, 114, 10252-10264.
(375) Theil, H. Indag. Math. 1950, 12, 173.
(376) Sen, P. K. J. Am. Stat. Assoc. 1968, 63, 1379-1389.
(377) Pedregosa, F. et al. J. Mach. Learn. Res. 2011, 12, 2825-2830.
(378) https://github.com/scikit-learn/scikit-learn, 2021.
(379) Pollice, R. et al. Matter 2021, 4, 1654-1682.
(380) Casanova-Páez, M.; Goerigk, L. J. Chem. Theory Comput. 2021, 17, 5165-5186.
(381) Nigam, A.; Pollice, R.; Aspuru-Guzik, A. arXiv: 2016.04011 2021.
(382) Pollice, R.; Ding, B.; Aspuru-Guzik, A. ChemRxiv 2022.
(383) Haase, F.; Roch, L. M.; Aspuru-Guzik, A. Chem. Sci. 2018, 9, 7642-7655.
(384) Pollice, R.; Friederich, P.; Lavigne, C.; Gomes, G. dos P.; Aspuru-Guzik, A. Matter 2021, 4, 1654-1682.

1. A compound of Formula V or a salt and/or tautomer thereof: 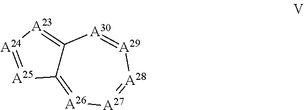
wherein:
A²³, A²⁴, A²⁵, A²⁶, A²⁷, A²⁸, A²⁹ and A³⁰ are each independently selected from C and N, and 0 to 4 of A²³, A²⁴, A²⁵, A²⁶, A²⁷, A²⁸, A²⁹ and A³⁰ are N; and
each of A²³, A²⁴, A²⁵, A²⁶, A²⁷, A²⁸, A²⁹ and A³⁰ that is a C is independently unsubstituted or substituted with a substituent of Formula Va
-L-R¹ Va
wherein:
L is selected from a bond, O, S, NH, —CR^{a}=, —N═, C(O), C₂₋₆alkenylene, C₂₋₆alkenyleneO and C₂₋₆alkenyleneS;
when L is a bond, R¹ is selected from H, CN and halo;
when L is other than a bond, R¹ is selected from H, C₅₋₂₀heteroaryl, C₅₋₂₀cycloalkenyl, C₆₋₂₀aryl, C₄₋₂₀heterocyclyl and C₂-C₆alkenyl, the latter 5 groups being unsubstituted or substituted with one or more groups independently selected from halo, CN, OH, NH₂, SH, C(O)H, C₂₋₄alkenyl, C₂₋₄fluoroalkenyl, NHC₂₋₄alkenyl, NHC₂₋₄fluoroalkenyl, OC₂₋₄alkenyl, SC₂₋₄fluoroalkenyl, NH₂-substituted C₂₋₄alkenyl, C(O)H, OC(O)H, NHC(O)H, SC(O)H, and C(O)NH₂, and/or
with one or two groups independently selected from =CHR^{a}, C₅-C₀heteroaryl, C₅₋₂₀cycloalkenyl, C₆₋₂₀aryl, C₄₋₂₀heterocyclyl, OC₅₋₂₀heteroaryl, OC₅₋₂₀cycloalkenyl, OC₆₋₂₀aryl, OC₄₋₂₀heterocyclyl, NHC₅₋₂₀heteroaryl, NHC₅₋₂₀cycloalkenyl, NHC₆₋₂₀aryl, NHC₄₋₂₀heterocyclyl, SC₅₋₂₀heteroaryl, SC₅₋₂₀cycloalkenyl, SC₆₋₂₀aryl, SC₄₋₂₀heterocyclyl, C(O)C₅₋₂₀heteroaryl, C(O)C₅₋₂₀cycloalkenyl, C(O)C₆₋₂₀aryl, C(O)C₄₋₂₀heterocyclyl, C₂₋₄alkenyleneC₅₋₂₀heteroaryl, C₂₋₄alkenyleneC₅₋₂₀cycloalkenyl, C₂₋₄alkenyleneC₆₋₂₀aryl and C₂₋₄alkenyleneC₄₋₂₀heterocyclyl, in which each cyclic group is unsubstituted or substituted with one or more groups independently selected from NH₂, CN, OH, SH, halo, C₂₋₄alkenyl and C(O)H;
R^{a} is selected from H, C₅₋₁₀heteroaryl, and C₆₋₁₀aryl, the latter two groups being unsubstituted or substituted with one or more group independently selected from NH₂, CN, OH, SH, halo, C₂₋₄alkenyl and C(O)H, and
the compound has a negative singlet-triplet gap and an oscillator strength greater than or equal to about 0.01.

2. The compound of claim 1, wherein 1 to 4, optionally 2 to 4, of A²³, A²⁴, A²⁵, A²⁶, A²⁷ A²⁸, A²⁹ and A³⁰ are N, or wherein A²⁴ is N, and/or one of A²³ and A²⁵ is N, or wherein at least one of A²⁹ and A³⁰ is N, and/or at least one of A²⁷ and A²⁹ is N.

3. The compound of claim 1, wherein the compound comprises 2 or 3 substituents of Formula Va, or wherein the compound comprises 2 substituents of Formula Va, optionally the 2 substituents of Formula Va are the same.

4. The compound of claim 1, wherein L is selected from a bond, O, S, NH, —CR^{a}=, —N═, C₂₋₄alkenylene, C₂₋₄alkenyleneO and C₂₋₄alkenyleneS, optionally selected from O, S, and NH.

5. The compound of claim 1, wherein L is other than a bond and R¹ is selected from C₅₋₁₀heteroaryl, C₅₋₁₀cycloalkenyl, C₆₋₁₀aryl, C₅₋₁₀heterocyclyl and C₂-C₄alkenyl, the latter 5 groups being unsubstituted or substituted with one to four groups independently selected from halo, CN, OH, NH₂, SH, C(O)H, C₂₋₄alkenyl, C₂₋₄fluoroalkenyl, NHC₂₋₄alkenyl, NHC₂₋₄fluoroalkenyl, OC₂₋₄alkenyl, SC₂₋₄fluoroalkenyl, NH₂-substituted C₂₋₄alkenyl, and C(O)H, and/or with one or two groups independently selected from =CHR^{a}, C₅-C₁₀heteroaryl, C₅₋₁₀cycloalkenyl, C₆₋₁₀aryl, C₄₋₁₀heterocyclyl, OC₅₋₁₀heteroaryl, OC₅₋₁₀cycloalkenyl, OC₆₋₂₀aryl, OC₄₋₁₀heterocyclyl, NHC₅₋₁₀heteroaryl, NHC₅₋₁₀cycloalkenyl, NHC₆₋₂₀aryl, NHC₄₋₁₀heterocyclyl, SC₅₋₁₀heteroaryl, SC₅₋₁₀cycloalkenyl, SC₆₋₁₀aryl, SC₄₋₁₀heterocyclyl, C(O)C₅₋₁₀heteroaryl, C(O)C₅₋₁₀cycloalkenyl, C(O)C₆₋₁₀aryl, C(O)C₄₋₁₀heterocycyl, C₂₋₄alkenyleneC₅₋₁₀heteroaryl, C₂₋₄alkenyleneC₅₋₁₀cycloalkenyl, C₂₋₄alkenyleneC₆₋₁₀aryl and C₂₋₄alkenyleneC₄₋₁₀heterocyclyl, in which each cyclic group is unsubstituted or substituted with one to five groups independently selected from NH₂, CN, OH, SH, halo, C₂₋₄alkenyl and C(O)H, or wherein L is other than a bond and R¹ is selected from C₅₋₁₀heteroaryl, C₅₋₁₀cycloalkenyl, C₆₋₁₀aryl, and C₅₋₁₀heterocyclyl, the latter 4 groups being unsubstituted or substituted with one to four groups independently selected from F, CN, OH, NH₂, SH, C(O)H, C₂alkenyl, C₂fluoroalkenyl, NHC₂alkenyl, NHC₂fluoroalkenyl, OC₂alkenyl, SC₂fluoroalkenyl, and C(O)H, and/or with one or two groups independently selected from =CHR^{a}, C₅-C₁₀heteroaryl, C₅₋₁₀cycloalkenyl, C₆₋₁₀aryl, C₄₋₁₀heterocyclyl, OC₅₋₁₀heteroaryl, OC₅₋₁₀cycloalkenyl, OC₆₋₂₀aryl, OC₄₋₁₀heterocyclyl, NHC₅₋₁₀heteroaryl, NHC₅₋₁₀cycloalkenyl, NHC₆₋₂₀aryl, NHC₄₋₁₀heterocyclyl, SC₅₋₁₀heteroaryl, SC₅₋₁₀cycloalkenyl, SC₆₋₁₀aryl, SC₄₋₁₀heterocyclyl, C(O)C₅₋₁₀heteroaryl, C(O)C₅₋₁₀cycloalkenyl, C(O)C₆₋₁₀aryl, C(O)C₄₋₁₀heterocycyl, C₂alkenyleneC₅₋₁₀heteroaryl, C₂alkenyleneC₅₋₁₀cycloalkenyl, C₂alkenyleneC₆₋₁₀aryl and C₂alkenyleneC₄₋₁₀heterocyclyl, in which each cyclic group is unsubstituted or substituted with one to four groups independently selected from NH₂, CN, OH, SH, halo, C₂₋₄alkenyl and C(O)H.

6. The compound of claim 1, wherein each R¹ is independently selected from aryl and heteroaryl, optionally wherein each R¹ is substituted with one to four substituents independently selected from OH, NH, SH, halo, and C₂-C₄alkene.

7. The compound of claim 1, wherein the compound is selected from the compounds shown in Table A or Table B, and salt and/or tautomer thereof.

8. The compound of claim 1, wherein the compound comprises 1 to 6 substituents of Formula Va.

9. The compound of claim 8, wherein each L is a bond and each R¹ is independently CN or NH₂.

10. Use of a compound of claim 1 in an organic light emitting diode, wherein the compound is used as an emitter or a dopant.

11. An organic light emitting diode comprising at least one compound of claim 1.

12. Method of preparing an organic light emitting diode comprising providing at least one compound of claim 1 as an emitter or a dopant.

13. Use of a compound of claim 1 as a photocatalyst.

14. Method of performing photocatalysis comprising contacting at least one compound of claim 1 with a mixture requiring a photocatalyst and performing a photocatalytic transformation on the mixture.

15. Use of a compound of claim 1 in the generation of organic laser.

16. Method of generating organic laser comprising providing at least one compound of claim 1 as a light emitter.

17. Use of a compound of claim 1 in the enhancement of photostability, optionally wherein the compound is used as a triplet quencher.

18. Method of enhancing photostability comprising providing at least one compound of claim 1 as a triplet quencher.

19. A photocatalyst comprising at least one compound of claim 1.

20. A triplet quencher comprising at least one compound of claim 1.
